(19) United States
(12) Patent Application Publication
(10) Pub. No.: US 2012/0150032 A1
(43) Pub. Date: Jun. 14, 2012

(54) Sequence Variants Associated with Prostate Specific Antigen Levels

(76) Inventors: Julius Gudmundsson, Reykjavik (IS); Daniel Gudbjartsson, Reykjavik (IS); Patrick Sulem, Reykjavik (IS)

(21) Appl. No.: 12/966,505

(22) Filed: Dec. 13, 2010

(51) Int. Cl. A61B 8/00; G06F 19/00; G01N 33/573; C12Q 1/68; C40B 30/00

(52) U.S. Cl. 600/437; 435/6.11; 506/7; 435/7.4; 435/6.14; 435/6.1; 702/19

(57) ABSTRACT

Certain sequence variants have been found to be useful for correcting Prostate Specific Antigen levels in humans. The invention provides diagnostic applications based on such correction, including methods of diagnosis of prostate cancer.

### BACKGROUND

Prostate cancer is among the leading causes of cancer death in men. In the US, prostate cancer has become the most frequent cause of cancer in men with more than 192,000 predicted new cases (25% of all new male cancer diagnoses) and 27,360 deaths (9% of all cancer deaths in men) in 2009. Early diagnosis and treatment are key factors in determining the survival and prognosis of prostate cancer patients, prompting intensive searches for biomarkers for screening.

Prostate-specific antigen (PSA) is a protein produced by the cells of prostate gland. PSA is present in small quantities in serum of men with a healthy prostate, but is often elevated in individuals with prostate cancer and other prostate disorders. A blood test to measure PSA is considered the most effective test currently available for the early detection of prostate cancer, although but its clinical effectiveness has been questioned. Rising levels of PSA over time are associated with both localized and metastatic prostate cancer. In general, PSA values ranging from 2.5 ng/mL to 4 ng/mL are considered as cut-off values for suspected cancer, and levels above 10 ng/mL indicate higher risk. However, despite the widespread use of the PSA screening test, it is limited both in specificity and sensitivity and substantial controversy exists about its beneficial effect for patients. This is mainly due to the fact that PSA is not a specific marker of prostate cancer since its serum levels increase in prostatic hyperplasia and are affected by many other factors such as medication, urologic manipulations and inflammation. Notably, a recent study showed that 47% of men with PSA levels between 10 and 50 ng/ml were not diagnosed with prostate cancer (3). Furthermore, not all individuals with prostate cancer have raised levels of PSA.

PSA levels in the population are known to be variable. One approach to increase the specificity and sensitivity of the PSA test is to work out a model that defines what is a “normal” PSA value for a given man. Genetic factors have been shown to account for as much as 40 to 45% of the variability in PSA levels among men in the general population.

Knowledge about genetic variants that affect PSA levels is important for establishing PSA levels that are considered normal, taking into account the genetic background of any given individual. The present invention provides methods for correcting PSA levels based on genetic factors.

### SUMMARY OF THE INVENTION

The present invention relates to methods for determining corrected PSA quantity in humans. The invention also provides methods for determining prostate cancer risk, and prognostic methods for prostate cancer.

In a first aspect, the invention provides a method of determining corrected PSA quantity in a human individual, the method comprising obtaining data identifying an uncorrected PSA quantity in a first biological sample from the human individual, analyzing sequence data about at least one polymorphic marker from the first biological sample or a second biological sample from the human individual, wherein the at least one polymorphic marker is correlated with PSA quantity in humans; and determining a corrected PSA quantity in the human individual based on the sequence data about the at least one polymorphic marker. In one embodiment, the at least one marker is selected from the group consisting of rs401681, rs2736098, rs10788160, rs11067228, rs10993994, rs4430796, rs2735839 and rs17632542, and markers in linkage disequilibrium therewith

In a second aspect, the invention provides a method of diagnosis of prostate cancer in a human individual, the method comprising (a) Detecting an uncorrected PSA quantity in a first biological sample from the human individual; (b) Obtaining sequence data about at least one polymorphic marker in the first biological sample or in a second biological sample from the human individual, wherein the at least one polymorphic marker is correlated with PSA quantity in humans; (c) Determining a corrected PSA quantity in the human individual based on the sequence data about the at least one polymorphic marker; (d) Determining whether the corrected PSA quantity is greater than normal PSA quantity in humans; and (e) Performing a further diagnostic evaluation procedure selected from the group consisting of rectal ultrasound imaging and prostate biopsy on the individual if the corrected PSA quantity is determined to be greater than the reference range; wherein determination of a positive outcome of the ultrasound imaging or prostate biopsy is indicative of prostate cancer in the individual.

Also provided is a method of determining a susceptibility to prostate cancer, the method comprising analyzing nucleic acid sequence data from a human individual for at least one polymorphic marker selected from the group consisting of rs17632542, and markers in linkage disequilibrium therewith, wherein different alleles of the at least one polymorphic marker are associated with different susceptibilities to prostate cancer in humans, and determining a susceptibility to prostate cancer from the nucleic acid sequence data.

Further provided is a method for identifying a human individual who is a candidate for further diagnostic evaluation for prostate cancer, the method comprising the steps of (a) obtaining data representing uncorrected values of PSA quantity in the individual; (b) determining, in the genome of the human individual, the allelic identity of at least one allele of at least one polymorphic marker, wherein different alleles of the at least one marker are associated with different levels of PSA quantity in humans, and wherein the at least one marker is selected from the group consisting of rs401681, rs2736098, rs10788160, rs11067228, rs10993994, rs4430796, rs2735839 and rs17632542, and markers in linkage disequilibrium therewith; (c) determining a corrected PSA quantity in the individual based on the allelic identity of the at least one polymorphic marker; and (d) identifying the subject as a subject who is a candidate for further diagnostic evaluation for prostate cancer if said corrected PSA quantity is greater than values of normal PSA quantity in humans.

The invention also relates to computer-implemented aspects. One such aspect provides an apparatus for determining PSA quantity in a human individual, comprising a processor, a computer-readable memory having instructions for execution on a processor, wherein the instructions relate to the determination of corrected PSA quantity for a human individual.

Further provided is a computer-readable medium that comprises data representing uncorrected PSA values, data comprising sequence data about at least one polymorphic marker predictive of PSA quantity in humans, and a routine stored on the medium for execution on a processor to determine corrected PSA values.

It should be understood that all combinations of features described herein are contemplated, even if the combination of feature is not specifically found in the same sentence or paragraph herein. This includes in particular the use of all markers disclosed herein, alone or in combination, for use in all aspects of the invention as described herein.

### BRIEF DESCRIPTION OF THE DRAWINGS

The foregoing and other objects, features and advantages of the invention will be apparent from the following more particular description of preferred embodiments of the invention.

FIG. 1 provides a diagram illustrating a computer-implemented system utilizing risk variants as described herein.

FIG. 2 shows the distribution of personalized PSA cutoff values after applying a genetic correction for the commonly used PSA cutoff of 4 ng/mL, based on the effect of four SNPs (rs2736098, rs10788160, rs11067228 and rs17632542) in samples from the Icelandic (ICE) and UK populations. The Y-axis indicates personalized PSA cutoff values (ng/mL) based on the correction for the four SNPs, and the X-axis indicates % of the distribution.

FIGS. 3A-3B show results for four biopsy outcome models. Shown are results from analyses of the area under the receiver-operating-characteristic curve (AUC) for four biopsy outcome models. The four different models included data on: 1) PSA levels (red line), 2) the combined prostate cancer risk prediction of 23 established sequence variants (green line), 3) genetic correction of PSA values based on the sequence variants rs2736098, rs10788160, rs11067228 and rs17632542 (blue line), 4) both the genetic correction of PSA levels and the combined risk of the 23 prostate cancer risk variants (pink line). The black diagonal line indicates random classification, for comparison to the four different models. (A) results from Iceland (n=415): AUC for model-1=70.4%, AUC for model-2=63.0%, AUC for model-3=70.9%, AUC for model-4=73.2%. (B) results from the UK (n=1,291): AUC for model-1=57.1%, AUC for model-2=61.1%, AUC for model-3=58.5%, AUC for model-4=63.3%.

### DETAILED DESCRIPTION

### Definitions

Unless otherwise indicated, nucleic acid sequences are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by the ordinary person skilled in the art to which the invention pertains.

The following terms shall, in the present context, have the meaning as indicated:

A “polymorphic marker”, sometime referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including SNPs, mini- or microsatellites, translocations and copy number variations (insertions, deletions, duplications). Polymorphic markers can be of any measurable frequency in the population. For mapping of disease genes, polymorphic markers with population frequency higher than 5-10% are in general most useful. However, polymorphic markers may also have lower population frequencies, such as 1-5% frequency, or even lower frequency, in particular copy number variations (CNVs). The term shall, in the present context, be taken to include polymorphic markers with any population frequency.

An “allele” refers to the nucleotide sequence of a given locus (position) on a chromosome. A polymorphic marker allele thus refers to the composition (i.e., sequence) of the marker on a chromosome. Genomic DNA from an individual contains two alleles (e.g., allele-specific sequences) for any given polymorphic marker, representative of each copy of the marker on each chromosome. Sequence codes for nucleotides used herein are: A=1, C=2, G=3, T=4. For microsatellite alleles, the CEPH sample (Centre d'Etudes du Polymorphisme Humain, genomics repository, CEPH sample 1347-02) is used as a reference, the shorter allele of each microsatellite in this sample is set as 0 and all other alleles in other samples are numbered in relation to this reference. Thus, e.g., allele 1 is 1 bp longer than the shorter allele in the CEPH sample, allele 2 is 2 bp longer than the shorter allele in the CEPH sample, allele 3 is 3 bp longer than the lower allele in the CEPH sample, etc., and allele-1 is 1 bp shorter than the shorter allele in the CEPH sample, allele-2 is 2 bp shorter than the shorter allele in the CEPH sample, etc.

Sequence conucleotide ambiguity as described herein is according to WIPO ST.25:

| IUB code | Meaning |
|---|---|
| A | Adenosine |
| C | Cytidine |
| G | Guanine |
| T | Thymidine |
| R | G or A |
| Y | T or C |
| K | G or T |
| M | A or C |
| S | G or C |
| W | A or T |
| B | C, G or T |
| D | A, G or T |
| H | A, C or T |
| V | A, C or G |
| N | A or G or C or T, unknown or other |

A nucleotide position at which more than one sequence is possible in a population (either a natural population or a synthetic population, e.g., a library of synthetic molecules) is referred to herein as a “polymorphic site”.

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

A “variant”, as described herein, refers to a segment of DNA that differs from the reference DNA. A “marker” or a “polymorphic marker”, as defined herein, is a variant. Alleles that differ from the reference are referred to as “variant” alleles.

A “microsatellite” is a polymorphic marker that has multiple small repeats of bases that are 2-8 nucleotides in length (such as CA repeats) at a particular site, in which the number of repeat lengths varies in the general population. An “indel” is a common form of polymorphism comprising a small insertion or deletion that is typically only a few nucleotides long.

A “haplotype,” as described herein, refers to a segment of genomic DNA that is characterized by a specific combination of alleles arranged along the segment. For diploid organisms such as humans, a haplotype comprises one member of the pair of alleles for each polymorphic marker or locus along the segment. In a certain embodiment, the haplotype can comprise two or more alleles, three or more alleles, four or more alleles, or five or more alleles.

Allelic identities are described herein in the context of the marker name and the particular allele of the marker, e.g., “4 rs17632542” refers to the 4 allele of marker rs17632542, and is equivalent to “rs17632542 allele 4”. Furthermore, allelic codes are as for individual markers, i.e. 1=A, 2=C, 3=G and 4=T.

The term “susceptibility”, as described herein, refers to the proneness of an individual towards the development of a certain state (e.g., a certain trait, phenotype or disease), or towards being less able to resist a particular state than the average individual. The term, also referred to as “risk”, encompasses both increased susceptibility and decreased susceptibility. Thus, particular alleles at polymorphic markers may be characteristic of increased susceptibility (i.e., increased risk) of prostate cancer, as characterized by a relative risk (RR) or odds ratio (OR) of greater than one for the particular allele. Alternatively, the markers are characteristic of decreased susceptibility (i.e., decreased risk) of prostate, as characterized by a relative risk of less than one.

The term “and/or” shall in the present context be understood to indicate that either or both of the items connected by it are involved. In other words, the term herein shall be taken to mean “one or the other or both”.

The term “look-up table”, as described herein, is a table that correlates one form of data to another form, or one or more forms of data to a predicted outcome to which the data is relevant, such as phenotype or trait. For example, a look-up table can comprise a correlation between allelic data for at least one polymorphic marker and a particular trait or phenotype, such as a particular disease diagnosis, that an individual who comprises the particular allelic data is likely to display, or is more likely to display than individuals who do not comprise the particular allelic data. Look-up tables can be multidimensional, i.e. they can contain information about multiple alleles for single markers simultaneously, or the can contain information about multiple markers, and they may also comprise other factors, such as particulars about diseases diagnoses, racial information, biomarkers, biochemical measurements, therapeutic methods or drugs, etc.

A “computer-readable medium”, is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

A “nucleic acid sample” as described herein, refers to a sample obtained from an individual that contains nucleic acid (DNA or RNA). In certain embodiments, i.e. the detection of specific polymorphic markers and/or haplotypes, the nucleic acid sample comprises genomic DNA. Such a nucleic acid sample can be obtained from any source that contains genomic DNA, including a blood sample, sample of amniotic fluid, sample of cerebrospinal fluid, or tissue sample from skin, muscle, buccal or conjunctival mucosa, placenta, gastrointestinal tract or other organs.

The term “antisense agent” or “antisense oligonucleotide” refers, as described herein, to molecules, or compositions comprising molecules, which include a sequence of purine an pyrimidine heterocyclic bases, supported by a backbone, which are effective to hydrogen bond to a corresponding contiguous bases in a target nucleic acid sequence. The backbone is composed of subunit backbone moieties supporting the purine an pyrimidine heterocyclic bases at positions which allow such hydrogen bonding. These backbone moieties are cyclic moieties of 5 to 7 atoms in size, linked together by phosphorous-containing linkage units of one to three atoms in length. In certain preferred embodiments, the antisense agent comprises an oligonucleotide molecule.

The term “quantity”, as described herein, refers to the amount or level of a particular compound or substance. For example, PSA quantity refers to the amount of PSA in a particular object or sample. The quantity may be determined as a mass or a molar quantity. The quantity may also suitably be reported as a concentration, for example as mass/volume or molar quantity/volume. As an example, PSA quantity is sometimes determined in units of ng/mL (nanograms per milliliter).

### Methods of Determining Corrected PSA Values

Although PSA is widely used as a screening test for prostate cancer, it is limited in both specificity and sensitivity. This is mainly due to the fact that PSA is not a specific marker for prostate cancer, since its levels increase due to other conditions, including prostatic hyperplasia, and PSA levels are also known to be affected by factors such as medication, urologic manipulation and inflammation. Further, it has been established that between 40 and 45% of the variability in PSA levels in the general population is due to inherited factors.

One approach to increase the specificity and sensitivity of the PSA test is to work out a model that defines what is a “normal” PSA value for a given human. Such a model would have to take into account a number of factors, including genetic variants. However, to date these genetic variants have remained largely unknown, and methods for applying such variants for correcting PSA values have not been established.

The present inventors have discovered that certain genetic variants are predictive of PSA levels in humans. Such variants determine in part normal PSA levels in humans. By applying information about the effect of genetic variants on PSA levels, methods to determine corrected PSA levels can be developed. Results from estimating the combined relative effect of variants shown herein to be associated with PSA levels demonstrate a considerable variation in PSA levels between individuals based on their genotypes. By applying the combined genetic effect on commonly used PSA cutoff values, a personalized PSA cutoff value can be obtained. The data indicate that for a substantial fraction of men undergoing PSA-based prostate cancer screening, the personalized PSA cutoff value (for the decision of doing a biopsy or not) is shifted and hence men would be reclassified with respect to whether or not they should undergo a biopsy. This reclassification is likely to affect both the sensitivity and the specificity of the PSA test, and thereby, also the long term outcome of the patients since early diagnosis is the most powerful way to improve the patient's prognosis. For a screening test as important and widely used as the PSA test, having a better way to interpret the measured PSA level is likely to improve substantially the clinical performance of the test.

As a consequence, methods are described herein for correcting PSA levels determined in humans to determine a PSA value that reflects the genetic composition of individuals at variants known to influence normal PSA levels.

Accordingly, the present invention provides a method of determining corrected PSA quantity in a human individual. Such a method may in one aspect comprise steps of
(a) Obtaining data identifying an uncorrected PSA quantity in a first sample from the human individual;
(b) Analyzing sequence data about at least one polymorphic marker from the first sample or a second sample from the human individual, wherein the at least one polymorphic marker is correlated with PSA quantity in humans; and
(c) Determining a corrected PSA quantity in the human individual based on the sequence data about the at least one polymorphic marker.

An “uncorrected” PSA quantity is in this context a quantity of PSA that is determined in a biological sample, and is not corrected or adjusted based on the presence, absence or magnitude of other substances in the sample. In one preferred embodiment, the uncorrected PSA quantity is a PSA quantity that has not been corrected based on the identity of genetic variants in the genome of the individual.

In certain embodiments, the human individual is a male individual.

In certain embodiments, the step of obtaining data identifying an uncorrected PSA quantity comprises detecting an uncorrected PSA quantity in a first sample from the human individual. The first sample is preferably a sample that comprises PSA protein. In certain embodiments, the sample is selected from the group consisting of a blood sample, a serum sample, a semen sample, a saliva sample, a urine sample, a prostate biopsy sample. Preferably, the sample is a serum sample. The sample may also be any other sample that contains PSA protein.

Determination of PSA quantity in human tissue can be done using any method available to the skilled person. Such methods include, but are not limited to, immunogenic tests such as Hybritech PSA test (Beckman Coulter) and Elecsys PSA assay (Roche). The skilled person will appreciate that the methods described herein are applicable for correction of PSA levels determined by any particular method that detects the amount or quantity of PSA protein.

Correction of PSA quantity is suitably done by using the determined allelic effect of any one allele of a polymorphic marker. For example, if a particular allele has been determined to lead to increased PSA levels by 15% in the population, then measured PSA values for an individual who carries one copy of the allele will be decreased by 15% to obtain a corrected PSA value. The effect of multiple markers in general can be assumed to be independent, and the multiplicative model applied.

As a consequence, the magnitude of the PSA correction obtained by the current method depends on the genotype of the individual for the markers are assessed to apply a genetic correction. In certain embodiments, the corrected PSA quantity differs from the uncorrected PSA quantity by at least 0.1 ng/mL. In certain embodiments, the corrected PSA quantity differs from the uncorrected PSA quantity by at least 0.5 ng/mL. In certain embodiments, the corrected PSA quantity differs from the uncorrected PSA quantity by at least 1.0 ng/mL. It will be appreciated that other values of the difference between uncorrected and corrected PSA values are possible and are also contemplated, including but not limited to at least 0.2 ng/mL, at least 0.3 ng/mL, at least 0.4 ng/mL, at least 0.6 ng/mL, at least 0.7 ng/mL, at least 0.8 ng/mL, at least 0.9 ng/mL, at least 1.1 ng/mL, and at least 1.2 ng/mL.

In certain embodiments, at least one allele of the at least one marker is predictive of an increased quantity of PSA in humans. In certain embodiments, at least one other allele of the at least one marker is predictive of a decreased quantity of PSA in humans. Thus, determining corrected PSA quantity in an individual comprises adjusting uncorrected PSA quantity based on the predicted effect of the particular alleles in the genome of the individual on PSA quantity in humans.

In certain embodiments, a further step is included, comprising preparing a report containing results from the determination of corrected PSA quantity. The report may be in any suitable format, including but not limited to a report written in a computer readable medium, printed on paper, or displayed on a visual display.

The skilled person will appreciate that for any polymorphic marker, the allele that is detected can be the allele of the complementary strand of DNA, such that the nucleic acid sequence data includes the identification of at least one allele which is complementary to any of the alleles of the polymorphic markers referenced above.

### Suitable Polymorphic Markers

The methods described herein for correcting PSA levels may be practiced using any one, or a combination of, polymorphic markers that are predictive of PSA levels in humans. The markers may be independent, i.e. in linkage equilibrium. The markers may also be in linkage disequilibrium. The skilled person will appreciate how to use any such marker in the methods described herein. In certain embodiments, if a marker is predictive of PSA levels in humans, at least one allele of the marker is predictive of increased PSA levels in humans, compared with the general population. Certain other allele(s) the marker may also be predictive of decreased PSA levels in humans. Identifying which allele(s) is predictive of increased PSA level, and which allele(s) is predictive of decreased PSA levels is a trivial exercise for the skilled person, once the marker has been identified, since a simple correlation with the particular allele(s) and PSA levels will in such cases be observed.

In preferred embodiments, markers useful for correcting PSA levels are selected from the group consisting of rs401681 (Which is identified in SEQ ID NO:1 herein), rs2736098 (SEQ ID NO:2), rs10788160 (SEQ ID NO:3), rs11067228 (SEQ ID NO:5), rs10993994 (SEQ ID NO:4), rs4430796 (SEQ ID NO:6), rs2735839 (SEQ ID NO:7) and rs17632542 (SEQ ID NO:8), and markers in linkage disequilibrium therewith.

In certain embodiments, the markers are selected from the group consisting of s.51165690, s.51172808, s.51175013, s.56037076, s.56054527, s.56058688, s.56060000, s.56066550, s.56066560, s.56066619, rs1058205, rs1061657, rs10749412, rs10749413, rs10763534, rs10763536, rs10763546, rs10763576, rs10763588, rs10788154, rs10788159, rs10788162, rs10788163, rs10788164, rs10788165, rs10788166, rs10788167, rs10825652, rs10826075, rs10826125, rs10826127, rs10886880, rs10886882, rs10886883, rs10886885, rs10886886, rs10886887, rs10886890, rs10886893, rs10886894, rs10886895, rs10886896, rs10886897, rs10886898, rs10886899, rs10886900, rs10886901, rs10886902, rs10886903, rs10908278, rs11004246, rs11004324, rs11004409, rs11004415, rs11004422, rs11004435, rs11006207, rs11006274, rs11199862, rs11199866, rs11199867, rs11199868, rs11199869, rs11199871, rs11199872, rs11199874, rs11199879, rs11199881, rs1125527, rs1125528, rs11263761, rs11263763, rs11593361, rs11598592, rs11599333, rs11609105, rs11651052, rs11651755, rs11657964, rs11658063, rs12146156, rs12146366, rs12413088, rs12413648, rs12415826, rs12761612, rs12763717, rs12781411, rs174776, rs17632542, rs1873450, rs1873451, rs1873452, rs2005705, rs2125770, rs2201026, rs2249986, rs2569735, rs2611489, rs2611506, rs2611507, rs2611508, rs2611509, rs2611512, rs2611513, rs2659051, rs2659122, rs2659124, rs266849, rs266878, rs27068, rs2735839, rs2735846, rs2735945, rs2736102, rs2736108, rs2843549, rs2843550, rs2843551, rs2843554, rs2843560, rs2843562, rs2901290, rs2926494, rs3101227, rs3123078, rs35716372, rs3741698, rs3744763, rs3760511, rs3925042, rs4131357, rs4237529, rs4239217, rs4304716, rs4306255, rs4393247, rs4465316, rs4468286, rs4486572, rs4489674, rs4512771, rs4554834, rs4581397, rs4630240, rs4630241, rs4630243, rs4631830, rs4752520, rs4935090, rs4935162, rs515746, rs545076, rs551510, rs567223, rs57263518, rs57858801, rs59336, rs62113216, rs6481329, rs67289834, rs7071471, rs7074985, rs7075009, rs7075697, rs7076500, rs7077830, rs7081532, rs7081844, rs7090326, rs7091083, rs7098889, rs7405696, rs7405776, rs7501939, rs7896156, rs7910704, rs7915008, rs7920517, rs7922901, rs7923130, rs8064454, rs8853, rs9630106, rs9787697, and rs9913260, which are the markers listed in Table 13 herein.

In certain embodiments, the markers are selected from the group consisting of rs2736098, rs10788160, rs11067228, rs10993994, rs4430796, and rs17632542, and markers in linkage disequilibrium therewith. In certain embodiments, the markers are selected from the group consisting of rs401681, rs2736098, rs10788160, rs17632542 and rs11067228, and markers in linkage disequilibrium therewith. In certain embodiments, the markers are selected from the group consisting of rs401681, rs2736098, rs10788160 and rs11067228, and markers in linkage disequilibrium therewith. In one embodiment, the markers are selected from the group consisting of rs2736098, and markers in linkage disequilibrium therewith. In one embodiment, the markers are selected from the group consisting of rs10788160, and markers in linkage disequilibrium therewith. In one embodiment, the markers are selected from the group consisting of rs11067228, and markers in linkage disequilibrium therewith. In one embodiment, the markers are selected from the group consisting of rs10993994, and markers in linkage disequilibrium therewith. In one embodiment, the markers are selected from the group consisting of rs4430796, and markers in linkage disequilibrium therewith. In one embodiment, the markers are selected from the group consisting of rs17632542, and markers in linkage disequilibrium therewith.

Certain alleles at these polymorphic markers are predictive of an increased PSA quantity in humans. In certain embodiments, determination of the presence of a marker allele selected from the group consisting of the C allele of rs401681, the A allele of rs2736098, the A allele of rs10788160, the T allele of rs10993994, the A allele of rs11067228, the A allele of rs4430796, the G allele of rs2735839 and the T allele of rs17632542 is indicative of elevated PSA quantity in the human individual. In one embodiment, the allele is the C allele of rs401681. In one embodiment, the allele is the A allele of rs2736098. In one embodiment, the allele is the A allele of rs10788160. In one embodiment, the allele is the T allele of rs10993994. In one embodiment, the allele is the A allele of rs11067228. In one embodiment, the allele is the A allele of rs4430796. In one embodiment, the allele is the G allele of rs2735839. In one embodiment, the allele is the T allele of rs17632542. Marker alleles in linkage disequilibrium with any one of these marker alleles are also predictive of increased PSA quantity in humans, and are therefore also useful in the methods described herein.

For example, a marker allele selected from the group consisting of s.51165690 allele C, s.51172808 allele G, s.51175013 allele A, s.56037076 allele T, s.56054527 allele T, s.56058688 allele T, s.56060000 allele A, s.56066550 allele T, s.56066560 allele C, s.56066619 allele G, rs1058205 allele T, rs1061657 allele T, rs10749412 allele T, rs10749413 allele T, rs10763534 allele C, rs10763536 allele G, rs10763546 allele C, rs10763576 allele A, rs10763588 allele G, rs10788154 allele C, rs10788159 allele G, rs10788162 allele G, rs10788163 allele G, rs10788164 allele T, rs10788165 allele G, rs10788166 allele T, rs10788167 allele A, rs10825652 allele A, rs10826075 allele G, rs10826125 allele G, rs10826127 allele G, rs10886880 allele C, rs10886882 allele T, rs10886883 allele G, rs10886885 allele T, rs10886886 allele G, rs10886887 allele T, rs10886890 allele G, rs10886893 allele C, rs10886894 allele C, rs10886895 allele A, rs10886896 allele A, rs10886897 allele C, rs10886898 allele G, rs10886899 allele T, rs10886900 allele G, rs10886901 allele C, rs10886902 allele C, rs10886903 allele G, rs10908278 allele A, rs11004246 allele C, rs11004324 allele G, rs11004409 allele C, rs11004415 allele A, rs11004422 allele G, rs11004435 allele A, rs11006207 allele T, rs11006274 allele T, rs11199862 allele A, rs11199866 allele A, rs11199867 allele T, rs11199868 allele A, rs11199869 allele G, rs11199871 allele A, rs11199872 allele A, rs11199874 allele A, rs11199879 allele C, rs11199881 allele C, rs1125527 allele A, rs1125528 allele A, rs11263761 allele A, rs11263763 allele A, rs11593361 allele A, rs11598592 allele A, rs11599333 allele C, rs11609105 allele A, rs11651052 allele G, rs11651755 allele T, rs11657964 allele G, rs11658063 allele G, rs12146156 allele C, rs12146366 allele T, rs12413088 allele T, rs12413648 allele A, rs12415826 allele C, rs12761612 allele A, rs12763717 allele G, rs12781411 allele T, rs174776 allele C, rs17632542 allele T, rs1873450 allele G, rs1873451 allele C, rs1873452 allele C, rs2005705 allele G, rs2125770 allele T, rs2201026 allele G, rs2249986 allele T, rs2569735 allele G, rs2611489 allele G, rs2611506 allele C, rs2611507 allele T, rs2611508 allele T, rs2611509 allele G, rs2611512 allele A, rs2611513 allele C, rs2659051 allele G, rs2659122 allele T, rs2659124 allele T, rs266849 allele A, rs266878 allele C, rs27068 allele C, rs2735839 allele G, rs2735846 allele G, rs2735945 allele C, rs2736102 allele C, rs2736108 allele T, rs2843549 allele C, rs2843550 allele C, rs2843551 allele C, rs2843554 allele G, rs2843560 allele G, rs2843562 allele C, rs2901290 allele A, rs2926494 allele T, rs3101227 allele C, rs3123078 allele C, rs35716372 allele A, rs3741698 allele C, rs3744763 allele A, rs3760511 allele G, rs3925042 allele T, rs4131357 allele C, rs4237529 allele G, rs4239217 allele A, rs4304716 allele A, rs4306255 allele A, rs4393247 allele A, rs4465316 allele A, rs4468286 allele A, rs4486572 allele A, rs4489674 allele G, rs4512771 allele C, rs4554834 allele A, rs4581397 allele A, rs4630240 allele G, rs4630241 allele G, rs4630243 allele T, rs4631830 allele C, rs4752520 allele T, rs4935090 allele T, rs4935162 allele G, rs515746 allele A, rs545076 allele A, rs551510 allele T, rs567223 allele T, rs57263518 allele A, rs57858801 allele T, rs59336 allele A, rs62113216 allele T, rs6481329 allele G, rs67289834 allele T, rs7071471 allele T, rs7074985 allele A, rs7075009 allele T, rs7075697 allele C, rs7076500 allele A, rs7077830 allele G, rs7081532 allele A, rs7081844 allele T, rs7090326 allele T, rs7091083 allele A, rs7098889 allele C, rs7405696 allele C, rs7405776 allele G, rs7501939 allele C, rs7896156 allele A, rs7910704 allele C, rs7915008 allele A, rs7920517 allele G, rs7922901 allele G, rs7923130 allele A, rs8064454 allele C, rs8853 allele C, rs9630106 allele G, rs9787697 allele C, rs9913260 allele G, rs1016990 allele C, rs17626423 allele C, rs2012677 allele A, and rs757210 allele G is predictive of increased PSA levels.

In certain embodiments, marker alleles selected from the group consisting of s.122837469 allele A, rs2130779 allele T, s.122876448 allele A, s.122901140 allele T, s.122901142 allele C, s.122905335 allele A, rs10788149 allele G, rs10749408 allele C, rs2172071 allele C, rs11592107 allele A, rs1907218 allele T, rs1907220 allele A, rs1994655 allele T, rs1907221 allele C, rs1907225 allele C, rs1907226 allele G, rs10749409 allele C, rs11199835 allele G, s.122991926 allele C, rs729014 allele T, s.122993518 allele G, s.122994309 allele A, s.122994946 allele G, rs1873450 allele G, rs2901290 allele A, s.122998594 allele A, s.122998678 allele T, s.122998978 allele T, rs2201026 allele G, rs4237529 allele G, s.122999386 allele G, rs1873451 allele C, rs1873452 allele C, rs4752520 allele T, rs10886880 allele C, rs10749412 allele T, s.123008216 allele A, rs3925042 allele T, rs1125527 allele A, rs1125528 allele A, rs4319451 allele G, rs10788154 allele C, rs7081844 allele T, rs7076500 allele A, s.123011774 allele T, s.123011879 allele T, rs11199862 allele A, s.123014171 allele C, rs12146156 allele C, s.123014499 allele G, s.123014519 allele A, rs12146366 allele T, s.123014684 allele A, rs7091083 allele A, rs7074985 allele A, rs7915008 allele A, s.123015342 allele A, s.123015365 allele A, rs10749413 allele T, rs11199866 allele A, s.123016003 allele A, rs7923130 allele A, rs7922901 allele G, rs10886882 allele T, rs10886883 allele G, rs11199867 allele T, s.123017698 allele T, s.123018111 allele C, rs4393247 allele A, s.123018188 allele T, rs4489674 allele G, rs11199868 allele A, s.123018670 allele T, s.123019408 allele G, s.123019759 allele G, rs11199869 allele G, s.123020245 allele T, s.123020365 allele T, rs10886885 allele T, rs10788159 allele G, rs10886886 allele G, rs11199871 allele A, rs11199872 allele A, rs12761612 allele A, rs4575197 allele G, rs11199874 allele A, rs10886887 allele T, s.123023625 allele T, s.123023836 allele C, rs4465316 allele A, rs4468286 allele A, rs10886890 allele G, rs10788162 allele G, s.123028135 allele A, rs12413648 allele A, s.123029102 allele C, rs10788163 allele G, s.123031617 allele T, s.123031811 allele T, rs10788164 allele T, rs11598592 allele A, rs10788165 allele G, rs9630106 allele G, rs10886893 allele C, s.123034821 allele C, rs11199879 allele C, rs11199881 allele C, rs12415826 allele C, rs10788166 allele G, rs10886894 allele C, rs10886895 allele A, rs10886896 allele A, rs10886897 allele C, rs10886898 allele G, rs10886899 allele T, rs10886900 allele G, rs10886901 allele C, rs10886902 allele C, rs10886903 allele G, rs12413088 allele T, rs10788167 allele A, s.123047182 allele T, rs7085073 allele T, rs7071101 allele A, rs12570783 allele A, rs11199884 allele A, rs7085506 allele G, rs10886905 allele C, rs10736302 allele C, s.123061811 allele T, s.123062031 allele C, rs11199886 allele T, s.123063327 allele T, s.123063715 allele A, rs10886907 allele C, s.123064252 allele T, s.123064345 allele T, s.123064780 allele T, s.123064783 allele C, s.123066424 allele C, s.123066700 allele C, rs3981043 allele T, rs11199896 allele T, rs11199897 allele A, rs11199898 allele C, s.123067963 allele A, rs11199900 allele T, rs11199901 allele T, s.123068178 allele T, s.123068222 allele A, s.123068236 allele T, s.123068424 allele G, s.123068619 allele T, s.123068743 allele G, s.123068926 allele T, s.123068997 allele A, s.123069012 allele T, s.123069326 allele T, s.123069570 allele T, s.123069989 allele C, s.123070105 allele T, s.123071090 allele A, s.123071347 allele C, rs4254007 allele A, s.123071495 allele A, s.123071914 allele T, s.123072804 allele A, rs7900630 allele T, s.123074016 allele C, rs1896416 allele A, s.123074531 allele T, s.123074928 allele T, s.123076274 allele C, s.123076472 allele G, rs2420925 allele C, s.123077398 allele G, s.123077455 allele C, rs12779205 allele T, rs11199912 allele T, rs4752534 allele C, s.123078389 allele T, rs1896420 allele T, rs1896419 allele C, s.123079199 allele A, s.123081990 allele A, s.123081993 allele A, s.123081998 allele G, s.123201870 allele C, s.51157005 allele G, s.51159221 allele C, rs35716372 allele A, s.51159373 allele C, s.51159376 allele C, s.51159399 allele T, s.51159786 allele C, rs4935090 allele T, rs12781411 allele T, s.51162137 allele G, s.51162792 allele A, s.51162795 allele A, rs11004246 allele C, s.51165690 allele C, rs11004324 allele G, rs2843562 allele C, rs11004409 allele C, rs11004415 allele A, rs11004422 allele G, s.51168415 allele T, rs11004435 allele A, rs11599333 allele C, s.51170094 allele G, s.51170307 allele A, rs12763717 allele G, rs67289834 allele T, s.51172442 allele A, s.51172558 allele G, rs57858801 allele T, s.51172618 allele A, s.51172808 allele G, s.51173184 allele G, rs7071471 allele T, rs7090326 allele T, s.51173565 allele G, s.51173983 allele C, s.51174391 allele G, s.51174499 allele C, s.51174610 allele T, s.51174944 allele A, s.51175013 allele A, s.51175409 allele G, s.51176290 allele T, s.51176963 allele C, s.51180209 allele A, rs10825652 allele A, s.51180819 allele A, rs2843560 allele G, rs2125770 allele T, rs2611513 allele C, rs2611512 allele A, rs2611509 allele G, s.51186305 allele G, rs2926494 allele T, rs2611508 allele T, rs2611507 allele T, s.51188694 allele A, rs2611506 allele C, rs57263518 allele A, s.51189522 allele G, rs3101227 allele C, rs2843549 allele C, rs2843550 allele C, rs2249986 allele T, rs2843551 allele C, s.51192126 allele C, rs7077830 allele G, s.51193219 allele A, rs2843554 allele G, s.51194280 allele C, rs2611489 allele G, rs3123078 allele C, rs4935162 allele G, rs7081532 allele A, rs10826075 allele G, rs7896156 allele A, s.51199599 allele A, rs6481329 allele G, rs7910704 allele C, rs4554834 allele A, rs10826125 allele G, rs10826127 allele G, rs4486572 allele A, rs4581397 allele A, rs4630240 allele G, rs7920517 allele G, rs4630241 allele G, rs9787697 allele C, rs10763534 allele C, rs10763536 allele G, s.51205998 allele C, rs10763546 allele C, s.51206890 allele C, rs4131357 allele C, s.51207437 allele C, s.51207481 allele G, s.51208175 allele A, rs11006207 allele T, rs10763576 allele A, s.51208921 allele G, rs11593361 allele A, rs10763588 allele G, rs11006274 allele T, s.51210619 allele A, s.51210866 allele G, rs4630243 allele T, rs4512771 allele C, rs4306255 allele A, s.51213076 allele T, rs4631830 allele C, rs7075009 allele T, rs7098889 allele C, rs4304716 allele A, s.51214689 allele A, s.51214690 allele T, rs7477953 allele G, s.51215034 allele G, s.51216121 allele A, s.51216342 allele A, rs7075697 allele C, s.51219226 allele C, s.51219227 allele T, s.51219230 allele C, s.51219320 allele T, s.51221179 allele C, s.113576401 allele A, s.113582477 allele G, s.113584188 allele G, s.113584539 allele G, s.113585097 allele T, rs12819162 allele A, rs11609105 allele A, rs514849 allele G, rs513061 allele T, s.113590733 allele A, rs1061657 allele T, rs8853 allele C, rs3741698 allele C, s.113594635 allele G, rs567223 allele T, rs551510 allele T, rs59336 allele A, s.113601412 allele G, rs515746 allele A, rs545076 allele A, s.113614584 allele C, rs3744763 allele A, rs7405776 allele G, rs2005705 allele G, s.33170591 allele T, rs11263761 allele A, rs4239217 allele A, rs11651755 allele T, rs10908278 allele A, s.33174083 allele T, rs11657964 allele G, rs7501939 allele C, rs8064454 allele C, s.33175746 allele T, s.33176039 allele A, rs7405696 allele C, rs11651052 allele G, rs11263763 allele A, rs11658063 allele G, rs9913260 allele G, rs3760511 allele G, s.33182344 allele C, s.55554247 allele A, s.55566277 allele T, s.55582344 allele C, rs2546552 allele G, s.55596785 allele T, s.55597645 allele A, s.55598078 allele A, s.55600121 allele A, s.55605246 allele G, s.55606024 allele A, s.55607242 allele G, s.55624341 allele C, s.55630396 allele T, s.55630578 allele T, s.55630679 allele T, s.55630791 allele T, s.55631170 allele C, s.55632347 allele A, s.55632363 allele A, s.55636052 allele T, s.55637350 allele C, s.55640040 allele T, s.55646568 allele A, s.55649132 allele T, s.55650629 allele A, s.55650844 allele G, s.55652397 allele G, s.55653401 allele T, s.55653991 allele A, s.55654907 allele A, s.55657973 allele G, s.55659043 allele A, s.55660011 allele G, s.55660013 allele T, s.55660139 allele T, s.55660143 allele T, s.55661660 allele C, s.55661718 allele T, rs6509476 allele A, s.55664020 allele G, s.55664897 allele T, s.55665723 allele G, s.55665726 allele G, s.55672641 allele C, s.55673254 allele G, s.55674252 allele G, s.55674254 allele A, s.55674727 allele T, s.55676073 allele A, s.55683393 allele G, s.55687122 allele A, s.55695317 allele A, s.55697027 allele C, s.55701748 allele C, rs7257447 allele T, s.55702308 allele A, s.55703568 allele T, s.55706751 allele T, s.55708051 allele T, s.55709067 allele A, s.55709498 allele T, s.55709766 allele T, s.55710030 allele C, s.55710848 allele T, s.55710851 allele A, s.55711749 allele A, s.55712802 allele G, s.55713451 allele T, s.55713453 allele G, s.55713458 allele C, s.55713862 allele T, s.55716007 allele G, s.55718272 allele A, s.55723496 allele C, s.55724346 allele T, s.55726794 allele G, s.55729556 allele A, s.55729562 allele G, s.55729563 allele A, s.55731588 allele G, s.55733658 allele G, s.55741403 allele C, s.55743524 allele T, s.55745833 allele A, s.55746123 allele T, s.55747079 allele T, s.55748269 allele T, s.55748274 allele T, s.55748844 allele T, s.55749193 allele G, s.55752178 allele T, s.55752271 allele A, s.55770158 allele A, rs7247686 allele T, s.55771401 allele T, s.55772266 allele C, s.55775314 allele C, s.55778756 allele G, s.55788661 allele G, s.55790622 allele T, s.55791942 allele A, rs10413426 allele G, s.55798366 allele G, s.55818900 allele G, s.55822129 allele C, s.55825528 allele G, s.55825624 allele T, s.55833489 allele T, s.55833938 allele G, s.55848124 allele G, s.55848125 allele G, s.55849044 allele A, s.55857289 allele T, s.55857585 allele A, s.55861107 allele G, s.55861111 allele A, s.55861196 allele T, s.55862851 allele T, s.55865439 allele T, s.55867208 allele A, s.55867650 allele G, s.55868902 allele G, s.55870429 allele C, rs73598616 allele G, s.55874339 allele T, s.55875249 allele C, s.55875725 allele C, s.55881262 allele A, s.55882788 allele T, s.55883542 allele C, s.55886467 allele T, s.55887498 allele T, s.55889175 allele G, s.55892113 allele A, s.55892618 allele T, s.55892866 allele T, s.55893305 allele G, s.55896443 allele G, s.55896826 allele A, s.55898241 allele T, s.55898245 allele A, s.55899120 allele T, s.55900597 allele G, s.55900764 allele A, s.55912567 allele T, s.55914840 allele A, s.55915776 allele G, s.55936192 allele T, s.55940336 allele C, s.55946316 allele G, s.55949971 allele C, s.55955333 allele G, s.55962188 allele T, s.55963864 allele G, s.55969754 allele T, s.55979135 allele T, rs67367861 allele C, s.55989580 allele A, s.56004001 allele A, s.56006528 allele G, s.56012046 allele G, s.56013739 allele G, rs2411330 allele G, rs3212825 allele G, s.56018053 allele G, s.56019106 allele C, rs7246740 allele A, s.56025860 allele G, s.56026713 allele T, rs55786312 allele T, s.56026881 allele A, s.56026882 allele A, s.56027319 allele A, s.56029265 allele C, s.56029362 allele G, s.56032778 allele G, s.56032963 allele T, s.56032964 allele G, s.56033138 allele G, s.56033138 allele G, s.56033664 allele T, s.56033664 allele T, s.56036363 allele G, s.56037076 allele T, s.56037076 allele T, rs2659051 allele G, s.56038334 allele A, s.56038334 allele A, s.56039736 allele C, rs266849 allele A, s.56042100 allele C, s.56042603 allele A, s.56042603 allele A, rs2659124 allele T, rs2659124 allele T, s.56046798 allele C, rs266878 allele C, rs266878 allele C, rs174776 allele C, rs174776 allele C, s.56052630 allele T, s.56052630 allele T, s.56052652 allele C, s.56052652 allele C, rs17632542 allele T, s.56053983 allele C, s.56054527 allele T, s.56054527 allele T, rs2659122 allele T, rs1058205 allele T, rs1058205 allele T, rs2569735 allele G, rs2569735 allele G, rs2735839 allele G, rs62113216 allele T, rs62113216 allele T, s.56058308 allele G, s.56058606 allele A, s.56058688 allele T, s.56058866 allele T, s.56060000 allele A, s.56061277 allele G, s.56062250 allele C, s.56066550 allele T, s.56066560 allele C, s.56066619 allele G, s.56067024 allele C, s.56067024 allele C, rs73592873 allele G, s.56076121 allele G, s.56076122 allele G, s.56078845 allele G, s.56085550 allele G, s.56093594 allele G, s.56472259 allele C, s.1030492 allele G, s.1233724 allele C, s.1251946 allele C, s.1257345 allele A, s.1258032 allele G, rs9418 allele T, s.1282167 allele T, s.1285240 allele T, s.1285775 allele A, s.1287049 allele A, s.1292191 allele C, s.1334730 allele A, s.1349759 allele T, s.1350079 allele A, rs2736108 allele T, s.1350854 allele T, rs2735948 allele G, rs2735846 allele G, s.1352392 allele G, s.1353401 allele C, rs2735946 allele G, rs2736102 allele C, rs2853666 allele A, rs2735945 allele C, s.1359165 allele C, rs4530805 allele C, s.1359765 allele G, rs61574973 allele C, s.1362904 allele A, s.1363152 allele A, rs12332579 allele T, rs6866783 allele C, s.1365329 allele C, rs13356727 allele A, rs13355267 allele C, s.1366701 allele G, rs10078017 allele T, rs4975615 allele A, rs4975616 allele A, rs6554759 allele A, rs3816659 allele G, rs1801075 allele T, rs451360 allele C, rs421629 allele G, rs380286 allele G, rs402710 allele C, rs10073340 allele C, rs414965 allele G, rs421284 allele T, rs466502 allele A, rs465498 allele A, rs452932 allele T, rs452384 allele T, rs370348 allele A, s.1386077 allele A, s.1386169 allele G, s.1386204 allele G, s.1386674 allele G, rs457130 allele A, rs467095 allele T, s.1389243 allele A, rs462608 allele T, rs456366 allele T, s.1390106 allele T, s.1390174 allele T, rs31487 allele G, s.1395154 allele T, rs31489 allele C, rs31490 allele G, rs27996 allele A, rs27071 allele T, rs27070 allele G, rs27068 allele C, s.1401106 allele T, rs37011 allele A, s.1402130 allele G, s.1402535 allele A, rs37009 allele C, rs40182 allele G, rs37008 allele G, rs37007 allele G, s.1407027 allele A, rs40181 allele G, s.1407682 allele A, rs37006 allele C, s.1408859 allele C, rs37005 allele C, s.1409771 allele A, rs37002 allele C, s.1411822 allele C, s.1411901 allele T, s.1412098 allele C, rs31494 allele G, s.1418662 allele T, s.1419748 allele G, s.1426206 allele T, s.1426336 allele T, s.1428371 allele A, s.1428373 allele A, s.1472454 allele T, s.1518154 allele C, s.1557827 allele A, rs11743119 allele C, s.1583465 allele A, rs4551123 allele G, s.1589581 allele G, s.1591616 allele C, s.1607388 allele T, rs6893515 allele T, s.1618305 allele C, s.1621550 allele C, s.1621551 allele A, rs6892057 allele G, s.1638061 allele C, rs6898387 allele C, rs7724451 allele G, rs2937006 allele A, s.1663985 allele T, s.1667254 allele A, s.1668831 allele T, s.1673499 allele A, s.1737379 allele G, s.1756873 allele A, s.1782909 allele G, s.1788485 allele C, s.1799150 allele A, s.1800043 allele T, s.1804565 allele A, s.1812409 allele G, s.886453 allele G, and s.887600 allele C, which are marker alleles as shown in Table 1, are indicative of increased PSA levels in the individual. These alleles are predicted to lead to elevated PSA levels in humans. Thus, a corrected PSA value for the individual for the particular marker allele will be lower than an uncorrected PSA value.

Certain other alleles at these markers are predictive of decreased PSA quantity in humans. In certain embodiments, marker alleles selected from the group consisting of the T allele of rs401681, the G allele of rs2736098, the G allele of rs10788160, the C allele of rs10993994, the G allele of rs11067228, the G allele of rs4430796, the A allele of rs2735839 and the C allele of rs17632542 are indicative of reduced PSA quantity in the individual.

In further embodiments, a marker allele selected from the group consisting of s.51165690 allele A, s.51172808 allele C, s.51175013 allele G, s.56037076 allele C, s.56054527 allele G, s.56058688 allele A, s.56060000 allele C, s.56066550 allele A, s.56066560 allele G, s.56066619 allele T, rs1058205 allele C, rs1061657 allele C, rs10749412 allele A, rs10749413 allele A, rs10763534 allele T, rs10763536 allele A, rs10763546 allele G, rs10763576 allele T, rs10763588 allele T, rs10788154 allele A, rs10788159 allele A, rs10788162 allele A, rs10788163 allele T, rs10788164 allele C, rs10788165 allele T, rs10788166 allele A, rs10788167 allele T, rs10825652 allele G, rs10826075 allele C, rs10826125 allele A, rs10826127 allele A, rs10886880 allele T, rs10886882 allele C, rs10886883 allele C, rs10886885 allele G, rs10886886 allele T, rs10886887 allele C, rs10886890 allele A, rs10886893 allele T, rs10886894 allele T, rs10886895 allele C, rs10886896 allele C, rs10886897 allele T, rs10886898 allele T, rs10886899 allele G, rs10886900 allele A, rs10886901 allele T, rs10886902 allele T, rs10886903 allele C, rs10908278 allele T, rs11004246 allele T, rs11004324 allele T, rs11004409 allele G, rs11004415 allele G, rs11004422 allele A, rs11004435 allele C, rs11006207 allele C, rs11006274 allele C, rs11199862 allele G, rs11199866 allele G, rs11199867 allele G, rs11199868 allele T, rs11199869 allele A, rs11199871 allele C, rs11199872 allele G, rs11199874 allele G, rs11199879 allele T, rs11199881 allele T, rs1125527 allele G, rs1125528 allele T, rs11263761 allele G, rs11263763 allele G, rs11593361 allele G, rs11598592 allele G, rs11599333 allele A, rs11609105 allele C, rs11651052 allele A, rs11651755 allele C, rs11657964 allele A, rs11658063 allele C, rs12146156 allele T, rs12146366 allele C, rs12413088 allele C, rs12413648 allele G, rs12415826 allele T, rs12761612 allele G, rs12763717 allele C, rs12781411 allele C, rs174776 allele T, rs17632542 allele C, rs1873450 allele T, rs1873451 allele T, rs1873452 allele T, rs2005705 allele A, rs2125770 allele C, rs2201026 allele T, rs2249986 allele G, rs2569735 allele A, rs2611489 allele A, rs2611506 allele T, rs2611507 allele C, rs2611508 allele A, rs2611509 allele A, rs2611512 allele G, rs2611513 allele T, rs2659051 allele C, rs2659122 allele C, rs2659124 allele A, rs266849 allele G, rs266878 allele G, rs27068 allele T, rs2735839 allele A, rs2735846 allele C, rs2735945 allele T, rs2736102 allele T, rs2736108 allele C, rs2843549 allele A, rs2843550 allele T, rs2843551 allele A, rs2843554 allele T, rs2843560 allele C, rs2843562 allele T, rs2901290 allele G, rs2926494 allele C, rs3101227 allele A, rs3123078 allele T, rs35716372 allele G, rs3741698 allele G, rs3744763 allele G, rs3760511 allele T, rs3925042 allele C, rs4131357 allele A, rs4237529 allele A, rs4239217 allele G, rs4304716 allele G, rs4306255 allele G, rs4393247 allele G, rs4465316 allele C, rs4468286 allele C, rs4486572 allele G, rs4489674 allele A, rs4512771 allele A, rs4554834 allele C, rs4581397 allele G, rs4630240 allele A, rs4630241 allele A, rs4630243 allele C, rs4631830 allele T, rs4752520 allele C, rs4935090 allele A, rs4935162 allele C, rs515746 allele G, rs545076 allele G, rs551510 allele C, rs567223 allele G, rs57263518 allele G, rs57858801 allele A, rs59336 allele T, rs62113216 allele A, rs6481329 allele A, rs67289834 allele C, rs7071471 allele C, rs7074985 allele T, rs7075009 allele G, rs7075697 allele G, rs7076500 allele G, rs7077830 allele C, rs7081532 allele G, rs7081844 allele C, rs7090326 allele A, rs7091083 allele G, rs7098889 allele T, rs7405696 allele G, rs7405776 allele A, rs7501939 allele T, rs7896156 allele G, rs7910704 allele T, rs7915008 allele G, rs7920517 allele A, rs7922901 allele C, rs7923130 allele G, rs8064454 allele A, rs8853 allele T, rs9630106 allele A, rs9787697 allele T, rs9913260 allele A, rs1016990 allele G, rs17626423 allele T, rs2012677 allele T, and rs757210 allele A is predictive of reduced PSA levels.

In certain embodiments, marker alleles selected from the group consisting of s.122837469 allele C, rs2130779 allele G, s.122876448 allele G, s.122901140 allele C, s.122901142 allele A, s.122905335 allele G, rs10788149 allele A, rs10749408 allele T, rs2172071 allele T, rs11592107 allele G, rs1907218 allele C, rs1907220 allele G, rs1994655 allele G, rs1907221 allele T, rs1907225 allele T, rs1907226 allele A, rs10749409 allele G, rs11199835 allele A, s.122991926 allele T, rs729014 allele C, s.122993518 allele A, s.122994309 allele G, s.122994946 allele T, rs1873450 allele T, rs2901290 allele G, s.122998594 allele G, s.122998678 allele G, s.122998978 allele A, rs2201026 allele T, rs4237529 allele A, s.122999386 allele A, rs1873451 allele T, rs1873452 allele T, rs4752520 allele C, rs10886880 allele T, rs10749412 allele A, s.123008216 allele G, rs3925042 allele C, rs1125527 allele G, rs1125528 allele T, rs4319451 allele A, rs10788154 allele A, rs7081844 allele C, rs7076500 allele G, s.123011774 allele C, s.123011879 allele C, rs11199862 allele G, s.123014171 allele T, rs12146156 allele T, s.123014499 allele A, s.123014519 allele G, rs12146366 allele C, s.123014684 allele C, rs7091083 allele G, rs7074985 allele T, rs7915008 allele G, s.123015342 allele C, s.123015365 allele G, rs10749413 allele A, rs11199866 allele G, s.123016003 allele G, rs7923130 allele G, rs7922901 allele C, rs10886882 allele C, rs10886883 allele C, rs11199867 allele G, s.123017698 allele C, s.123018111 allele G, rs4393247 allele G, s.123018188 allele C, rs4489674 allele A, rs11199868 allele T, s.123018670 allele G, s.123019408 allele T, s.123019759 allele C, rs11199869 allele A, s.123020245 allele G, s.123020365 allele A, rs10886885 allele G, rs10788159 allele A, rs10886886 allele T, rs11199871 allele C, rs11199872 allele G, rs12761612 allele G, rs4575197 allele A, rs11199874 allele G, rs10886887 allele C, s.123023625 allele G, s.123023836 allele T, rs4465316 allele C, rs4468286 allele C, rs10886890 allele A, rs10788162 allele A, s.123028135 allele C, rs12413648 allele G, s.123029102 allele T, rs10788163 allele T, s.123031617 allele G, s.123031811 allele A, rs10788164 allele C, rs11598592 allele G, rs10788165 allele T, rs9630106 allele A, rs10886893 allele T, s.123034821 allele T, rs11199879 allele T, rs11199881 allele T, rs12415826 allele T, rs10788166 allele A, rs10886894 allele T, rs10886895 allele C, rs10886896 allele C, rs10886897 allele T, rs10886898 allele T, rs10886899 allele G, rs10886900 allele A, rs10886901 allele T, rs10886902 allele T, rs10886903 allele C, rs12413088 allele C, rs10788167 allele T, s.123047182 allele C, rs7085073 allele C, rs7071101 allele G, rs12570783 allele G, rs11199884 allele G, rs7085506 allele C, rs10886905 allele T, rs10736302 allele T, s.123061811 allele C, s.123062031 allele G, rs11199886 allele G, s.123063327 allele A, s.123063715 allele G, rs10886907 allele G, s.123064252 allele C, s.123064345 allele G, s.123064780 allele C, s.123064783 allele T, s.123066424 allele T, s.123066700 allele T, rs3981043 allele A, rs11199896 allele C, rs11199897 allele G, rs11199898 allele T, s.123067963 allele T, rs11199900 allele A, rs11199901 allele C, s.123068178 allele G, s.123068222 allele G, s.123068236 allele C, s.123068424 allele A, s.123068619 allele C, s.123068743 allele A, s.123068926 allele A, s.123068997 allele G, s.123069012 allele C, s.123069326 allele G, s.123069570 allele C, s.123069989 allele T, s.123070105 allele C, s.123071090 allele G, s.123071347 allele G, rs4254007 allele T, s.123071495 allele G, s.123071914 allele G, s.123072804 allele G, rs7900630 allele C, s.123074016 allele T, rs1896416 allele G, s.123074531 allele C, s.123074928 allele C, s.123076274 allele T, s.123076472 allele C, rs2420925 allele T, s.123077398 allele A, s.123077455 allele G, rs12779205 allele A, rs11199912 allele G, rs4752534 allele T, s.123078389 allele A, rs1896420 allele C, rs1896419 allele A, s.123079199 allele G, s.123081990 allele T, s.123081993 allele T, s.123081998 allele A, s.123201870 allele T, s.51157005 allele A, s.51159221 allele T, rs35716372 allele G, s.51159373 allele T, s.51159376 allele G, s.51159399 allele G, s.51159786 allele G, rs4935090 allele A, rs12781411 allele C, s.51162137 allele A, s.51162792 allele C, s.51162795 allele C, rs11004246 allele T, s.51165690 allele A, rs11004324 allele T, rs2843562 allele T, rs11004409 allele G, rs11004415 allele G, rs11004422 allele A, s.51168415 allele C, rs11004435 allele C, rs11599333 allele A, s.51170094 allele T, s.51170307 allele G, rs12763717 allele C, rs67289834 allele C, s.51172442 allele T, s.51172558 allele T, rs57858801 allele A, s.51172618 allele C, s.51172808 allele C, s.51173184 allele A, rs7071471 allele C, rs7090326 allele A, s.51173565 allele C, s.51173983 allele T, s.51174391 allele A, s.51174499 allele A, s.51174610 allele C, s.51174944 allele G, s.51175013 allele G, s.51175409 allele A, s.51176290 allele C, s.51176963 allele T, s.51180209 allele G, rs10825652 allele G, s.51180819 allele C, rs2843560 allele C, rs2125770 allele C, rs2611513 allele T, rs2611512 allele G, rs2611509 allele A, s.51186305 allele T, rs2926494 allele C, rs2611508 allele A, rs2611507 allele C, s.51188694 allele C, rs2611506 allele T, rs57263518 allele G, s.51189522 allele A, rs3101227 allele A, rs2843549 allele A, rs2843550 allele T, rs2249986 allele G, rs2843551 allele A, s.51192126 allele T, rs7077830 allele C, s.51193219 allele T, rs2843554 allele T, s.51194280 allele T, rs2611489 allele A, rs3123078 allele T, rs4935162 allele C, rs7081532 allele G, rs10826075 allele C, rs7896156 allele G, s.51199599 allele C, rs6481329 allele A, rs7910704 allele T, rs4554834 allele C, rs10826125 allele A, rs10826127 allele A, rs4486572 allele G, rs4581397 allele G, rs4630240 allele A, rs7920517 allele A, rs4630241 allele A, rs9787697 allele T, rs10763534 allele T, rs10763536 allele A, s.51205998 allele T, rs10763546 allele G, s.51206890 allele A, rs4131357 allele A, s.51207437 allele T, s.51207481 allele A, s.51208175 allele C, rs11006207 allele C, rs10763576 allele T, s.51208921 allele T, rs11593361 allele G, rs10763588 allele T, rs11006274 allele C, s.51210619 allele C, s.51210866 allele A, rs4630243 allele C, rs4512771 allele A, rs4306255 allele G, s.51213076 allele G, rs4631830 allele T, rs7075009 allele G, rs7098889 allele T, rs4304716 allele G, s.51214689 allele G, s.51214690 allele C, rs7477953 allele A, s.51215034 allele A, s.51216121 allele G, s.51216342 allele G, rs7075697 allele G, s.51219226 allele G, s.51219227 allele G, s.51219230 allele G, s.51219320 allele C, s.51221179 allele T, s.113576401 allele T, s.113582477 allele A, s.113584188 allele A, s.113584539 allele A, s.113585097 allele C, rs12819162 allele G, rs11609105 allele C, rs514849 allele A, rs513061 allele C, s.113590733 allele C, rs1061657 allele C, rs8853 allele T, rs3741698 allele G, s.113594635 allele T, rs567223 allele G, rs551510 allele C, rs59336 allele T, s.113601412 allele T, rs515746 allele G, rs545076 allele G, s.113614584 allele G, rs3744763 allele G, rs7405776 allele A, rs2005705 allele A, s.33170591 allele C, rs11263761 allele G, rs4239217 allele G, rs11651755 allele C, rs10908278 allele T, s.33174083 allele C, rs11657964 allele A, rs7501939 allele T, rs8064454 allele A, s.33175746 allele G, s.33176039 allele G, rs7405696 allele G, rs11651052 allele A, rs11263763 allele G, rs11658063 allele C, rs9913260 allele A, rs3760511 allele T, s.33182344 allele T, s.55554247 allele G, s.55566277 allele C, s.55582344 allele G, rs2546552 allele T, s.55596785 allele G, s.55597645 allele T, s.55598078 allele C, s.55600121 allele T, s.55605246 allele T, s.55606024 allele C, s.55607242 allele A, s.55624341 allele A, s.55630396 allele C, s.55630578 allele C, s.55630679 allele C, s.55630791 allele C, s.55631170 allele A, s.55632347 allele T, s.55632363 allele T, s.55636052 allele C, s.55637350 allele A, s.55640040 allele C, s.55646568 allele G, s.55649132 allele C, s.55650629 allele C, s.55650844 allele C, s.55652397 allele A, s.55653401 allele C, s.55653991 allele T, s.55654907 allele C, s.55657973 allele A, s.55659043 allele G, s.55660011 allele A, s.55660013 allele C, s.55660139 allele A, s.55660143 allele A, s.55661660 allele T, s.55661718 allele A, rs6509476 allele C, s.55664020 allele C, s.55664897 allele A, s.55665723 allele C, s.55665726 allele C, s.55672641 allele T, s.55673254 allele A, s.55674252 allele C, s.55674254 allele T, s.55674727 allele A, s.55676073 allele T, s.55683393 allele A, s.55687122 allele T, s.55695317 allele T, s.55697027 allele A, s.55701748 allele A, rs7257447 allele A, s.55702308 allele T, s.55703568 allele A, s.55706751 allele A, s.55708051 allele A, s.55709067 allele T, s.55709498 allele G, s.55709766 allele A, s.55710030 allele G, s.55710848 allele A, s.55710851 allele T, s.55711749 allele G, s.55712802 allele C, s.55713451 allele G, s.55713453 allele T, s.55713458 allele A, s.55713862 allele A, s.55716007 allele T, s.55718272 allele T, s.55723496 allele T, s.55724346 allele C, s.55726794 allele T, s.55729556 allele C, s.55729562 allele T, s.55729563 allele C, s.55731588 allele A, s.55733658 allele T, s.55741403 allele G, s.55743524 allele G, s.55745833 allele T, s.55746123 allele C, s.55747079 allele G, s.55748269 allele A, s.55748274 allele C, s.55748844 allele G, s.55749193 allele A, s.55752178 allele C, s.55752271 allele T, s.55770158 allele G, rs7247686 allele C, s.55771401 allele C, s.55772266 allele G, s.55775314 allele A, s.55778756 allele C, s.55788661 allele A, s.55790622 allele C, s.55791942 allele G, rs10413426 allele A, s.55798366 allele T, s.55818900 allele C, s.55822129 allele T, s.55825528 allele A, s.55825624 allele G, s.55833489 allele C, s.55833938 allele A, s.55848124 allele C, s.55848125 allele C, s.55849044 allele G, s.55857289 allele G, s.55857585 allele T, s.55861107 allele T, s.55861111 allele C, s.55861196 allele C, s.55862851 allele C, s.55865439 allele C, s.55867208 allele T, s.55867650 allele T, s.55868902 allele A, s.55870429 allele G, rs73598616 allele T, s.55874339 allele A, s.55875249 allele G, s.55875725 allele A, s.55881262 allele T, s.55882788 allele G, s.55883542 allele T, s.55886467 allele G, s.55887498 allele A, s.55889175 allele A, s.55892113 allele G, s.55892618 allele A, s.55892866 allele A, s.55893305 allele C, s.55896443 allele A, s.55896826 allele T, s.55898241 allele G, s.55898245 allele T, s.55899120 allele C, s.55900597 allele A, s.55900764 allele C, s.55912567 allele C, s.55914840 allele G, s.55915776 allele T, s.55936192 allele G, s.55940336 allele T, s.55946316 allele A, s.55949971 allele G, s.55955333 allele A, s.55962188 allele A, s.55963864 allele A, s.55969754 allele A, s.55979135 allele A, rs67367861 allele T, s.55989580 allele T, s.56004001 allele G, s.56006528 allele C, s.56012046 allele T, s.56013739 allele A, rs2411330 allele C, rs3212825 allele C, s.56018053 allele T, s.56019106 allele A, rs7246740 allele T, s.56025860 allele A, s.56026713 allele C, rs55786312 allele A, s.56026881 allele G, s.56026882 allele G, s.56027319 allele G, s.56029265 allele A, s.56029362 allele T, s.56032778 allele C, s.56032963 allele G, s.56032964 allele T, s.56033138 allele A, s.56033138 allele A, s.56033664 allele A, s.56033664 allele A, s.56036363 allele T, s.56037076 allele C, s.56037076 allele C, rs2659051 allele C, s.56038334 allele G, s.56038334 allele G, s.56039736 allele G, rs266849 allele G, s.56042100 allele G, s.56042603 allele G, s.56042603 allele G, rs2659124 allele A, rs2659124 allele A, s.56046798 allele T, rs266878 allele G, rs266878 allele G, rs174776 allele T, rs174776 allele T, s.56052630 allele C, s.56052630 allele C, s.56052652 allele T, s.56052652 allele T, rs17632542 allele C, s.56053983 allele G, s.56054527 allele G, s.56054527 allele G, rs2659122 allele C, rs1058205 allele C, rs1058205 allele C, rs2569735 allele A, rs2569735 allele A, rs2735839 allele A, rs62113216 allele A, rs62113216 allele A, s.56058308 allele A, s.56058606 allele T, s.56058688 allele A, s.56058866 allele C, s.56060000 allele C, s.56061277 allele C, s.56062250 allele A, s.56066550 allele A, s.56066560 allele G, s.56066619 allele T, s.56067024 allele T, s.56067024 allele T, rs73592873 allele A, s.56076121 allele C, s.56076122 allele C, s.56078845 allele C, s.56085550 allele C, s.56093594 allele T, s.56472259 allele A, s.1030492 allele A, s.1233724 allele G, s.1251946 allele G, s.1257345 allele G, s.1258032 allele A, rs9418 allele C, s.1282167 allele C, s.1285240 allele C, s.1285775 allele T, s.1287049 allele G, s.1292191 allele T, s.1334730 allele C, s.1349759 allele C, s.1350079 allele C, rs2736108 allele C, s.1350854 allele C, rs2735948 allele A, rs2735846 allele C, s.1352392 allele A, s.1353401 allele T, rs2735946 allele T, rs2736102 allele T, rs2853666 allele G, rs2735945 allele T, s.1359165 allele T, rs4530805 allele T, s.1359765 allele C, rs61574973 allele T, s.1362904 allele G, s.1363152 allele G, rs12332579 allele C, rs6866783 allele T, s.1365329 allele T, rs13356727 allele G, rs13355267 allele T, s.1366701 allele A, rs10078017 allele C, rs4975615 allele G, rs4975616 allele G, rs6554759 allele G, rs3816659 allele A, rs1801075 allele C, rs451360 allele A, rs421629 allele A, rs380286 allele A, rs402710 allele T, rs10073340 allele T, rs414965 allele A, rs421284 allele C, rs466502 allele G, rs465498 allele G, rs452932 allele C, rs452384 allele C, rs370348 allele G, s.1386077 allele G, s.1386169 allele A, s.1386204 allele A, s.1386674 allele C, rs457130 allele T, rs467095 allele C, s.1389243 allele G, rs462608 allele A, rs456366 allele C, s.1390106 allele A, s.1390174 allele C, rs31487 allele C, s.1395154 allele C, rs31489 allele A, rs31490 allele A, rs27996 allele G, rs27071 allele C, rs27070 allele C, rs27068 allele T, s.1401106 allele C, rs37011 allele T, s.1402130 allele C, s.1402535 allele G, rs37009 allele T, rs40182 allele A, rs37008 allele A, rs37007 allele C, s.1407027 allele G, rs40181 allele T, s.1407682 allele T, rs37006 allele T, s.1408859 allele T, rs37005 allele T, s.1409771 allele C, rs37002 allele T, s.1411822 allele T, s.1411901 allele C, s.1412098 allele T, rs31494 allele T, s.1418662 allele C, s.1419748 allele A, s.1426206 allele A, s.1426336 allele C, s.1428371 allele C, s.1428373 allele C, s.1472454 allele C, s.1518154 allele A, s.1557827 allele C, rs11743119 allele G, s.1583465 allele T, rs4551123 allele A, s.1589581 allele C, s.1591616 allele G, s.1607388 allele C, rs6893515 allele C, s.1618305 allele G, s.1621550 allele T, s.1621551 allele G, rs6892057 allele C, s.1638061 allele T, rs6898387 allele T, rs7724451 allele A, rs2937006 allele G, s.1663985 allele G, s.1667254 allele G, s.1668831 allele C, s.1673499 allele G, s.1737379 allele A, s.1756873 allele C, s.1782909 allele A, s.1788485 allele G, s.1799150 allele G, s.1800043 allele G, s.1804565 allele G, s.1812409 allele A, s.886453 allele A, and s.887600 allele T, which are marker alleles listed in Table 1 herein, are indicative of reduced PSA levels in the individual. These alleles are predicted to lead to reduced PSA levels. Thus, a corrected PSA value for the individual for the particular marker allele will be greater than an uncorrected PSA value.

### Methods of Diagnosing Prostate Cancer

Prostate Specific Antigen (PSA) is a protein that is secreted by the epithelial cells of the prostate gland, including cancer cells. PSA is concentrated in prostatic tissue, and serum PSA levels are normally very low. Disruption of the normal prostate architecture, for example by prostatic disease, inflammation or trauma, allows greater amounts of PSA to enter the circulation. Thus, an elevated level in the blood indicates an abnormal condition of the prostate, either benign or malignant. PSA is used to detect potential problems in the prostate gland and to follow the progress of prostate cancer therapy.

After the introduction of PSA testing, a dramatic increase in diagnosis of prostate cancer was observed. Subsequently, a gradual decline in prostate cancer mortality in the US has been observed (Ries, L. A., et al. SEER Cancer Statistics Review, 1975-2005, National Cancer Institute, Bethesda, Md., http://seer.cancer.gov/csr/1975-2005/). Most cases of prostate cancer in the US are identified based on results of PSA testing. There is also evidence that PSA screening has led to a substantial shift towards detection of prostate cancer at earlier stages (Etzioni, R., et al. Med Decis Making 28:323 (2008)). Recent studies have also indicated that there is a modest reduction in prostate cancer deaths among those screened for PSA compared with those that were not (Schroder, F. H., et al. N Engl J Med 360:11320-8 (2009); Andriole, G. L. et al. N Engl J Med 360:1310-19 (2009)). A cutoff of 4 ng/mL PSA in human serum is typically used for selection of individuals for further screening, including prostate biopsy.

The decision to proceed with prostate biopsy is usually made based on results of a PSA assay, which is sometimes also followed by a Digital Rectal Examination (DRE). Results of PSA assay, alone or in combination with results of DRE, are used to select those individuals for prostate biopsy. Further factors may be considered, including free and total PSA, age of the patient, the rate of PSA change with age (PSA velocity), family history, ethnicity, history of prior biopsy and combordity.

Currently, the specificity of PSA testing using a cutoff level of 4 ng/mL is about 60 to 70% (Brawer, M. K., CA Cancer J Clin 49:264 (1999)). Because PSA levels tend to increase with age, ranging from 0-2.5 ng/mL in individuals age 40-49 to 0-6.5 ng/mL in individuals age 70-79 (Caucasians), it has been suggested that a higher “normal” value of PSA should be used for older individuals. However, it is clear that such increase in the applied cutoff values will lead to increased number of missed cancers in older men.

Prostate cancer is not limited to men with high PSA values. On the contrary, it has been found that even with men with PSA levels below 4.0 ng/mL, prostate cancer is fairly common (Thompson, I. M., et al. N Engl J Med 350:2239 (2004)), and in fact as much as 50 to 80% of prostate cancer is missed by applying this cutoff. Thus, while widespread PSA testing has been criticized as leading to overdetection of prostate cancer, possibly leading to overtreatment, it is also clear that many cases of prostate cancer are silent to current guidelines of PSA testing. As a consequence, biopsies are sometimes also done at lower PSA levels than 4 ng/mL.

Since it is known that PSA levels vary considerably in the population, and that this variation is to a large extent due to genetic factors, it is likely that a correction of PSA values of any particular individual based on the individual's genotype at genetic markers known to affect PSA levels could lead to significantly improved utility—through increased specificity and sensitivity—of PSA screening for reducing prostate cancer mortality in the population.

Correcting PSA levels by the methods described herein may in certain cases lead to corrected PSA values that are below the cutoff applied (such as 4 ng/mL), even though the uncorrected PSA value is above the threshold. This means that some individuals, who otherwise would undergo further diagnostic evaluation might not be selected for such follow-up, since it is likely that their increased uncorrected PSA value is due to natural fluctuations in PSA levels in the population rather than an actual underlying disease. However, in some cases corrected PSA values will be significantly higher than uncorrected values, and this could mean that individuals who normally would not be selected for further follow-up because their uncorrected PSA level is below the threshold applied for further clinical evaluation would, based on the corrected PSA values, be considered at risk for prostate cancer and thus selected for further evaluation. For example, let's consider a case where an individual is determined to have an uncorrected PSA value of 3.0. If this individual is determined not to carry the T allele of rs17632542, which leads to significantly elevated PSA levels (39-100% increase per allele), i.e. the individual is homozygous for the alternate C allele of rs17632542, then it is clear that the individual's PSA level is lower compared with the population in general because of the lack of the T allele in the individual's genome. The T allele is very common in the population (91% in Iceland, 93% in the UK), which means that the average PSA levels in the population are greatly affected by this allele. The corrected PSA value for this particular individual would be above the threshold of 4.0 that is routinely used for screening, and therefore the individual would undergo further testing, either DRE or biopsy, or both.

As further illustrated herein, the benefit of applying a correction to observed (uncorrected) PSA levels can be striking. For example, when considering the exemplary data as described in Example 2 herein, the personalized cutoff value of 4 ng/mL is in some cases shifted dramatically when correction for variants affecting PSA levels is applied. Thus, in the particular example shown in Example 2 herein, in certain cases some individuals with apparent PSA levels of 4.0 ng/mL, the corrected PSA value in those individuals may be as high as 5-8 ng/mL or as low as 1-2 ng/mL. Further examples illustrating the usefulness of applying the PSA correction are described in Example 5 and Example 6 herein.

Thus, corrected PSA levels as determined by the methods described herein could have enormous implications for the management of prostate cancer, since PSA screening based on PSA values corrected for genetic background will better reflect physical changes in the individual (e.g., prostate cancer or other prostate disease) than do uncorrected PSA values, which may be largely dominated by inherent PSA levels, and not necessarily representing underlying disease.

As a consequence, the present invention provides diagnostic applications based on the determination of corrected PSA quantity. In one such application, a method of diagnostic evaluation of prostate cancer in a human individual is provided, the method comprising:
(a) Detecting an uncorrected PSA quantity in a first sample from the human individual;
(b) Obtaining sequence data about at least one polymorphic marker in the first sample or in a second sample from the human individual, wherein the at least one polymorphic marker is correlated with PSA levels in humans;
(c) Determining a corrected PSA quantity in the human individual based on the sequence data about the at least one polymorphic marker;
(d) Comparing the corrected PSA quantity determined in (c) with a reference range of normal PSA quantity in humans;
   wherein determination of a corrected PSA quantity that is greater than the reference range is indicative of suspected prostate cancer in the individual.

In another aspect, the invention provides a method of diagnosis of prostate cancer in humans, the method comprising:
(a) Obtaining an uncorrected PSA quantity in a first biological sample from the human individual;
(b) Obtaining sequence data about at least one polymorphic marker in the first biological sample or in a second biological sample from the human individual, wherein the at least one polymorphic marker is correlated with PSA quantity in humans;
(c) Determining a corrected PSA quantity in the human individual based on the sequence data about the at least one polymorphic marker;
(d) Determining whether the corrected PSA quantity is greater than normal PSA quantity in humans;
(e) Performing a further diagnostic evaluation procedure selected from the group consisting of rectal ultrasound imaging and prostate biopsy on the individual if the corrected PSA quantity is determined to be greater than the reference range;
   wherein determination of a positive outcome of the ultrasound imaging or prostate biopsy is indicative of prostate cancer in the individual.

In certain embodiments, the obtaining of uncorrected PSA quantity comprises detecting the PSA quantity in a first biological sample from the individual.

A further aspect provides a method of diagnosis of prostate cancer, the method comprising: Analyzing corrected PSA quantity of a human individual, wherein if the corrected PSA levels of the human individual are determined to be greater than normal PSA quantity in humans, a further diagnostic evaluation selected from the group consisting of rectal ultrasound imaging and prostate biopsy is performed; and wherein determination of a positive outcome of the further diagnostic evaluation is indicative of prostate cancer in the individual. Preferably, the corrected PSA quantity is determined using any one of the methods of determining corrected PSA quantity described herein.

A further diagnostic application relates to selection processes for individuals who are undergoing evaluation for prostate cancer. For example, an individual who is a candidate for further diagnostic evaluation for prostate cancer can be selected by (a) obtaining data representing uncorrected values of PSA quantity in the individual; (b) determining, in the genome of the human individual, the allelic identity of at least one allele of at least one polymorphic marker, wherein different alleles of the at least one marker are associated with different levels of PSA quantity in humans, and wherein the at least one marker is selected from the group consisting of rs401681, rs2736098, rs10788160, rs11067228, rs10993994, rs4430796, rs2735839 and rs17632542, and markers in linkage disequilibrium therewith; (c) determining a corrected PSA quantity in the individual based on the allelic identity of the at least one polymorphic marker; and (d) identifying the subject as a subject who is a candidate for further diagnostic evaluation for prostate cancer if said corrected PSA quantity is greater than values of normal PSA quantity in humans.

The invention further provides methods of treatment of prostate cancer diagnosed by the diagnostic methods described herein. Thus, methods of diagnosing prostate cancer as described herein may in certain embodiment comprise an additional step of treatment of prostate cancer, wherein the treatment is selected from the group consisting of surgery, radiation therapy, proton therapy, hormonal therapy and chemotherapy.

A further aspect of the invention relates to a method of treatment of prostate cancer, the method comprising (i) determining a corrected PSA quantity in the individual, wherein the corrected PSA quantity is determined based on the allelic identity of at least one allele of at least one polymorphic marker, wherein different alleles of the at least one marker are associated with different levels of PSA quantity in humans, and wherein the at least one marker is selected from the group consisting of rs401681, rs2736098, rs10788160, rs11067228, rs10993994, rs4430796, rs2735839 and rs17632542, and markers in linkage disequilibrium therewith; and (ii) performing a prostate biopsy if the corrected PSA quantity is greater than values of normal PSA quantity in humans; wherein if the individual is determined to have prostate cancer based on the prostate biopsy, the individual is selected for at least one treatment module selected from the group consisting of surgery, radiation therapy, proton therapy, hormonal therapy and chemotherapy.

The range of normal PSA quantity in humans may in certain embodiments by less than 50 ng/mL, less than 40 ng/mL, less than 30 ng/mL, less than 20 ng/mL, less than 10 ng/mL, less than 9 ng/mL, less than 8 ng/mL, less than 7 ng/mL, less than 6 ng/mL, less than 5 ng/mL, less than 4 ng/mL, less than 3.5 ng/mL, less than 3.0 ng/mL, less than 2.5 ng/mL, less than 2.0 ng/mL, less than 1.5 ng/mL, less than 1.0 ng/mL or less than 0.5 ng/mL. In one preferred embodiment, normal PSA quantity in humans is less than 4.0 ng/mL. In another preferred embodiment, normal PSA quantity in humans is less than 3.5 ng/mL. In another preferred embodiment, normal PSA quantity is less than 3.0 ng/mL. In another preferred embodiment, normal PSA quantity is less than 2.5 ng/mL. Other appropriate cutoff values bridging any of the above numbers may also be suitably be selected as appropriate values for normal PSA levels in humans.

In certain cases, the human individual is in a particular age group. For example, the individual may be less than age 40, the individual may be age 40-49, age 50-59, age 60-69, age 70-79, age 70 or higher. In certain such embodiments, the normal PSA quantity is determined in the same age group as the individual. For example, if the individual is in the age 40-49, the reference value of normal PSA quantity in humans is suitably determined in individuals age 40-49. The invention is applicable to any particular age range, and all age ranges are contemplated and within scope of the invention. In preferred embodiments, normal PSA values are determined in the same age range as the individual who is undergoing diagnostic evaluation.
In preferred embodiments, PSA is determined in human blood samples, in particular in human serum. However, the present invention is applicable for correcting PSA levels determined in any human tissue.

### Methods of Determining a Susceptibility to Prostate Cancer

The present invention also provides methods of determining a susceptibility to prostate cancer. It has been discovered that allele T of the marker rs17632542 is indicative of increased susceptibility of prostate cancer in humans (OR=1.39; P-value 1.8×10⁻¹⁰). This marker, and other markers in linkage disequilibrium therewith, is therefore useful for determining a susceptibility to prostate cancer.

As a consequence, in one aspect the invention provides a method of determining a susceptibility to prostate cancer, the method comprising analyzing nucleic acid sequence data from a human individual for at least one polymorphic marker selected from the group consisting of rs17632542, and markers in linkage disequilibrium therewith, wherein different alleles of the at least one polymorphic marker are associated with different susceptibilities to prostate cancer in humans, and determining a susceptibility to prostate cancer from the nucleic acid sequence data.

In certain embodiments, markers in linkage disequilibrium with rs17632542 are in linkage disequilibrium as characterized by values of r² with rs17632542 of 0.2 or greater. In certain embodiments, markers in linkage disequilibrium with rs17632542 are selected from the group consisting of s.55554247, s.55566277, s.55582344, rs2546552, s.55596785, s.55597645, s.55598078, s.55600121, s.55605246, s.55606024, s.55607242, s.55624341, s.55630396, s.55630578, s.55630679, s.55630791, s.55631170, s.55632347, s.55632363, s.55636052, s.55637350, s.55640040, s.55646568, s.55649132, s.55650629, s.55650844, s.55652397, s.55653401, s.55653991, s.55654907, s.55657973, s.55659043, s.55660011, s.55660013, s.55660139, s.55660143, s.55661660, s.55661718, rs6509476, s.55664020, s.55664897, s.55665723, s.55665726, s.55672641, s.55673254, s.55674252, s.55674254, s.55674727, s.55676073, s.55683393, s.55687122, s.55695317, s.55697027, s.55701748, rs7257447, s.55702308, s.55703568, s.55706751, s.55708051, s.55709067, s.55709498, s.55709766, s.55710030, s.55710848, s.55710851, s.55711749, s.55712802, s.55713451, s.55713453, s.55713458, s.55713862, s.55716007, s.55718272, s.55723496, s.55724346, s.55726794, s.55729556, s.55729562, s.55729563, s.55731588, s.55733658, s.55741403, s.55743524, s.55745833, s.55746123, s.55747079, s.55748269, s.55748274, s.55748844, s.55749193, s.55752178, s.55752271, s.55770158, rs7247686, s.55771401, s.55772266, s.55775314, s.55778756, s.55788661, s.55790622, s.55791942, rs10413426, s.55798366, s.55818900, s.55822129, s.55825528, s.55825624, s.55833489, s.55833938, s.55848124, s.55848125, s.55849044, s.55857289, s.55857585, s.55861107, s.55861111, s.55861196, s.55862851, s.55865439, s.55867208, s.55867650, s.55868902, s.55870429, rs73598616, s.55874339, s.55875249, s.55875725, s.55881262, s.55882788, s.55883542, s.55886467, s.55887498, s.55889175, s.55892113, s.55892618, s.55892866, s.55893305, s.55896443, s.55896826, s.55898241, s.55898245, s.55899120, s.55900597, s.55900764, s.55912567, s.55914840, s.55915776, s.55936192, s.55940336, s.55946316, s.55949971, s.55955333, s.55962188, s.55963864, s.55969754, s.55979135, rs67367861, s.55989580, s.56004001, s.56006528, s.56012046, s.56013739, rs2411330, rs3212825, s.56018053, s.56019106, rs7246740, s.56025860, s.56026713, rs55786312, s.56026881, s.56026882, s.56027319, s.56029265, s.56029362, s.56032778, s.56032963, s.56032964, s.56033138, s.56033138, s.56033664, s.56033664, s.56036363, s.56037076, s.56037076, s.56038334, s.56038334, s.56039736, s.56042100, s.56042603, s.56042603, rs2659124, rs2659124, s.56046798, rs266878, rs266878, rs174776, rs174776, s.56052630, s.56052630, s.56052652, s.56052652, s.56053983, s.56054527, s.56054527, rs1058205, rs1058205, rs2569735, rs2569735, rs2735839, rs62113216, rs62113216, s.56058308, s.56058606, s.56058688, s.56058866, s.56060000, s.56061277, s.56062250, s.56066550, s.56066560, s.56066619, s.56067024, s.56067024, rs73592873, s.56076121, s.56076122, s.56078845, s.56085550, s.56093594, s.56472259, and rs273622.

In certain embodiments, determination of the presence of the T allele of rs17632542 is indicative of increased susceptibility to prostate cancer in the individual. Other marker alleles indicative of increased susceptibility to prostate cancer may also be suitably selected using the information provided in Table 1. In certain embodiments, marker alleles indicative of increased susceptibility in humans are selected from the group consisting of s.55554247 allele A, s.55566277 allele T, s.55582344 allele C, rs2546552 allele G, s.55596785 allele T, s.55597645 allele A, s.55598078 allele A, s.55600121 allele A, s.55605246 allele G, s.55606024 allele A, s.55607242 allele G, s.55624341 allele C, s.55630396 allele T, s.55630578 allele T, s.55630679 allele T, s.55630791 allele T, s.55631170 allele C, s.55632347 allele A, s.55632363 allele A, s.55636052 allele T, s.55637350 allele C, s.55640040 allele T, s.55646568 allele A, s.55649132 allele T, s.55650629 allele A, s.55650844 allele G, s.55652397 allele G, s.55653401 allele T, s.55653991 allele A, s.55654907 allele A, s.55657973 allele G, s.55659043 allele A, s.55660011 allele G, s.55660013 allele T, s.55660139 allele T, s.55660143 allele T, s.55661660 allele C, s.55661718 allele T, rs6509476 allele A, s.55664020 allele G, s.55664897 allele T, s.55665723 allele G, s.55665726 allele G, s.55672641 allele C, s.55673254 allele G, s.55674252 allele G, s.55674254 allele A, s.55674727 allele T, s.55676073 allele A, s.55683393 allele G, s.55687122 allele A, s.55695317 allele A, s.55697027 allele C, s.55701748 allele C, rs7257447 allele T, s.55702308 allele A, s.55703568 allele T, s.55706751 allele T, s.55708051 allele T, s.55709067 allele A, s.55709498 allele T, s.55709766 allele T, s.55710030 allele C, s.55710848 allele T, s.55710851 allele A, s.55711749 allele A, s.55712802 allele G, s.55713451 allele T, s.55713453 allele G, s.55713458 allele C, s.55713862 allele T, s.55716007 allele G, s.55718272 allele A, s.55723496 allele C, s.55724346 allele T, s.55726794 allele G, s.55729556 allele A, s.55729562 allele G, s.55729563 allele A, s.55731588 allele G, s.55733658 allele G, s.55741403 allele C, s.55743524 allele T, s.55745833 allele A, s.55746123 allele T, s.55747079 allele T, s.55748269 allele T, s.55748274 allele T, s.55748844 allele T, s.55749193 allele G, s.55752178 allele T, s.55752271 allele A, s.55770158 allele A, rs7247686 allele T, s.55771401 allele T, s.55772266 allele C, s.55775314 allele C, s.55778756 allele G, s.55788661 allele G, s.55790622 allele T, s.55791942 allele A, rs10413426 allele G, s.55798366 allele G, s.55818900 allele G, s.55822129 allele C, s.55825528 allele G, s.55825624 allele T, s.55833489 allele T, s.55833938 allele G, s.55848124 allele G, s.55848125 allele G, s.55849044 allele A, s.55857289 allele T, s.55857585 allele A, s.55861107 allele G, s.55861111 allele A, s.55861196 allele T, s.55862851 allele T, s.55865439 allele T, s.55867208 allele A, s.55867650 allele G, s.55868902 allele G, s.55870429 allele C, rs73598616 allele G, s.55874339 allele T, s.55875249 allele C, s.55875725 allele C, s.55881262 allele A, s.55882788 allele T, s.55883542 allele C, s.55886467 allele T, s.55887498 allele T, s.55889175 allele G, s.55892113 allele A, s.55892618 allele T, s.55892866 allele T, s.55893305 allele G, s.55896443 allele G, s.55896826 allele A, s.55898241 allele T, s.55898245 allele A, s.55899120 allele T, s.55900597 allele G, s.55900764 allele A, s.55912567 allele T, s.55914840 allele A, s.55915776 allele G, s.55936192 allele T, s.55940336 allele C, s.55946316 allele G, s.55949971 allele C, s.55955333 allele G, s.55962188 allele T, s.55963864 allele G, s.55969754 allele T, s.55979135 allele T, rs67367861 allele C, s.55989580 allele A, s.56004001 allele A, s.56006528 allele G, s.56012046 allele G, s.56013739 allele G, rs2411330 allele G, rs3212825 allele G, s.56018053 allele G, s.56019106 allele C, rs7246740 allele A, s.56025860 allele G, s.56026713 allele T, rs55786312 allele T, s.56026881 allele A, s.56026882 allele A, s.56027319 allele A, s.56029265 allele C, s.56029362 allele G, s.56032778 allele G, s.56032963 allele T, s.56032964 allele G, s.56033138 allele G, s.56033138 allele G, s.56033664 allele T, s.56033664 allele T, s.56036363 allele G, s.56037076 allele T, s.56037076 allele T, s.56038334 allele A, s.56038334 allele A, s.56039736 allele C, s.56042100 allele C, s.56042603 allele A, s.56042603 allele A, rs2659124 allele T, rs2659124 allele T, s.56046798 allele C, rs266878 allele C, rs266878 allele C, rs174776 allele C, rs174776 allele C, s.56052630 allele T, s.56052630 allele T, s.56052652 allele C, s.56052652 allele C, s.56053983 allele C, s.56054527 allele T, s.56054527 allele T, rs1058205 allele T, rs1058205 allele T, rs2569735 allele G, rs2569735 allele G, rs2735839 allele G, rs62113216 allele T, rs62113216 allele T, s.56058308 allele G, s.56058606 allele A, s.56058688 allele T, s.56058866 allele T, s.56060000 allele A, s.56061277 allele G, s.56062250 allele C, s.56066550 allele T, s.56066560 allele C, s.56066619 allele G, s.56067024 allele C, s.56067024 allele C, rs73592873 allele G, s.56076121 allele G, s.56076122 allele G, s.56078845 allele G, s.56085550 allele G, s.56093594 allele G, s.56472259 allele C, and rs273622 allele A.

Determination of the absence of at least one of the at-risk alleles recited above is indicative of a decreased risk of prostate cancer for the human individual. As a consequence, in certain embodiments, the analyzing comprises determining the presence or absence of at least one at-risk allele of the polymorphic marker. Individuals who are homozygous for at-risk alleles are at particularly high risk. Thus, in certain embodiments determination of the presence of two alleles of one or more of the above-recited risk alleles is indicative of particularly high risk (susceptibility) of prostate cancer.

Alternatively, the allele that is detected can be the allele of the complementary strand of DNA. This means that that the nucleic acid sequence data may include the identification of at least one allele which is complementary to any of the alleles of the polymorphic markers referenced above.

In certain embodiments, the nucleic acid sequence data is obtained from a biological sample containing nucleic acid from the human individual. The nucleic acids sequence may suitably be obtained using a method that comprises at least one procedure selected from (i) amplification of nucleic acid from the biological sample; (ii) hybridization assay using a nucleic acid probe and nucleic acid from the biological sample; and (iii) hybridization assay using a nucleic acid probe and nucleic acid obtained by amplification of the biological sample. The nucleic acid sequence data may also be obtained from a preexisting record. For example, the preexisting record may comprise a genotype dataset for at least one polymorphic marker. In certain embodiments, the determining comprises comparing the sequence data to a database containing correlation data between the at least one polymorphic marker and susceptibility to the condition.

It is contemplated that in certain embodiments of the invention, it may be convenient to prepare a report of results of risk assessment. Thus, certain embodiments of the methods of the invention comprise a further step of preparing a report containing results from the determination, wherein said report is written in a computer readable medium, printed on paper, or displayed on a visual display. In certain embodiments, it may be convenient to report results of susceptibility to at least one entity selected from the group consisting of the individual, a guardian of the individual, a genetic service provider, a physician, a medical organization, and a medical insurer.

In certain embodiments, determination of the presence of at least one copy of the T allele of rs17632542 in the genome of an individual is indicative of increased risk of prostate cancer with an early age of onset. In other embodiments, determination of the presence of at least one copy of a marker allele in linkage disequilibrium with the T allele of rs17632542 is indicative of increased risk of prostate cancer with an early age of onset. Individuals who are homozygous for such risk alleles are at particularly increased risk of prostate cancer with an early onset. In certain embodiments, the age of onset of prostate cancer is below 50 years. In certain embodiments, the age of onset of prostate cancer is below 45 years. In certain embodiments, the age of onset of prostate cancer is below 40 years.

An individual who is at an increased susceptibility (i.e., increased risk) for prostate cancer is an individual in whom at least one specific allele at one or more polymorphic marker, or haplotype, conferring increased susceptibility (increased risk) for the disease is identified (i.e., at-risk marker alleles or haplotypes). The at-risk marker or haplotype is one that confers an increased risk (increased susceptibility) of the disease. In one embodiment, significance associated with a marker or—is measured by a relative risk (RR). In another embodiment, significance associated with a marker or haplotype is measured by an odds ratio (OR). In a further embodiment, the significance is measured by a percentage. In one embodiment, a significant increased risk is measured as a risk (relative risk and/or odds ratio) of at least 1.1, including but not limited to: at least 1.15, at least 1.20, at least 1.25, at least 1.30, at least 1.35, at least 1.40, at least 1.45, at least 1.5, at least 1.6, at least 1.7, at least 1.8, at least 1.9, and at least 2.0. In a particular embodiment, a risk (relative risk and/or odds ratio) of at least 1.2 is significant. In another particular embodiment, a risk of at least 1.30 is significant. In yet another embodiment, a risk of at least 1.35 is significant. In a further embodiment, a relative risk of at least 1.5 is significant. However, other cutoffs are also contemplated, e.g., at least 1.15, 1.25, 1.35, and so on, and such cutoffs are also within scope of the present invention. In other embodiments, a significant increase in risk is at least about 20%, including but not limited to about 20%, 25%, 30%, 35%, 40%, 45%, 50%, 60%, 70%, 80%, 90%, and 100%. In certain embodiments, a significant increase in risk is characterized by a p-value, such as a p-value of less than 0.05, less than 0.01, less than 0.001, less than 0.0001, less than 0.00001, less than 0.000001, less than 0.0000001, less than 0.00000001, or less than 0.000000001.

An at-risk polymorphic marker as described herein is one where at least one allele of at least one marker or haplotype is more frequently present in an individual at risk for prostate cancer (affected), or diagnosed with prostate cancer, compared to the frequency of its presence in a comparison group (control), such that the presence of the at least one allele of the at least one marker or haplotype is indicative of susceptibility to prostate cancer. The control group may in one embodiment be a population sample, i.e. a random sample from the general population. In another embodiment, the control group is represented by a group of individuals who are disease-free, i.e. not diagnosed with prostate cancer.

The person skilled in the art will appreciate that for markers with two alleles present in the population being studied (such as SNPs), and wherein one allele is found in increased frequency in a group of individuals with a trait or disease in the population, compared with controls, the other allele of the marker will be found in decreased frequency in the group of individuals with the trait or disease, compared with controls. In such a case, one allele of the marker (the one found in increased frequency in individuals with the trait or disease) will be the at-risk allele, while the other allele will be a protective allele.

Thus, in other embodiments of the invention, an individual who is at a decreased susceptibility (i.e., at a decreased risk) for prostate cancer is an individual in whom at least one specific allele at one or more polymorphic marker or haplotype conferring decreased susceptibility for prostate cancer is identified. The marker alleles conferring decreased risk are also said to be protective. In one aspect, the protective marker or haplotype is one that confers a significant decreased risk (or susceptibility) of prostate cancer. In one embodiment, significant decreased risk is measured as a relative risk (or odds ratio) of less than 0.9, including but not limited to less than 0.8, less than 0.7, less than 0.6, and less than 0.5. In one particular embodiment, significant decreased risk is less than 0.80. In another embodiment, significant decreased risk is less than 0.75. In yet another embodiment, significant decreased risk is less than 0.70. In another embodiment, the decrease in risk (or susceptibility) is at least 20%, including but not limited to at least 25%, at least 30%, at least 35%, at least 40%, at least 45%, and at least 50%. Other cutoffs or ranges as deemed suitable by the person skilled in the art to characterize the invention are however also contemplated, and those are also within scope of the present invention.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affected and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/(fᵢ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

### Number of Polymorphic Markers/Genes Analyzed

With regard to the methods described herein, the methods can comprise obtaining sequence data about any number of polymorphic markers and/or about any number of genes. For example, the method can comprise obtaining sequence data for about at least 1, 2, 3, 4, 5, 6, 7, 8, 9, 10, 15, 20, 25, 30, 40, 50, 100, 500, 1000, 10,000 or more polymorphic markers. The markers can be independent and/or the markers may be in linkage disequilibrium. The markers may also form a haplotype. The polymorphic markers can be the ones of the group specified herein or they can be different polymorphic markers that are not listed herein, including, for example, polymorphic markers in linkage disequilibrium with the markers described herein. In a specific embodiment, the method comprises obtaining sequence data about at least two polymorphic markers. In certain embodiments, each of the markers may be associated with a different gene. For example, in some instances, if the method comprises obtaining nucleic acid data about a human individual identifying at least one allele of a polymorphic marker, then the method comprises identifying at least one allele of at least one polymorphic marker. Also, for example, the method can comprise obtaining sequence data about a human individual identifying alleles of multiple, independent markers or haplotypes, which are not in linkage disequilibrium. In another specific embodiment of the invention, the method comprises obtaining nucleic acid sequence data about at least one polymorphic marker from associated with at least one gene selected from the group consisting of the KLK3 gene, the HNF1B gene, the FGFR2 gene, the TBX3 gene, the MSMB gene and the TERT gene.

### Obtaining Nucleic Acid Sequence Data

Sequence data can be nucleic acid sequence data, which may be obtained by means known in the art. For example, nucleic acid sequence data may be obtained through direct analysis of the sequence of the polymorphic position (allele) of a polymorphic marker. Suitable methods, some of which are described herein, include, for instance, whole genome analysis using a whole genome SNP chip (e.g., Infinium HD BeadChip), cloning for polymorphisms, non-radioactive PCR-single strand conformation polymorphism analysis, denaturing high pressure liquid chromatography (DHPLC), DNA hybridization, computational analysis, single-stranded conformational polymorphism (SSCP), restriction fragment length polymorphism (RFLP), automated fluorescent sequencing; clamped denaturing gel electrophoresis (CDGE); denaturing gradient gel electrophoresis (DGGE), mobility shift analysis, restriction enzyme analysis; heteroduplex analysis, chemical mismatch cleavage (CMC), RNase protection assays, use of polypeptides that recognize nucleotide mismatches, such as E. coli mutS protein, allele-specific PCR, and direct manual and automated sequencing. These and other methods are described in the art (see, for instance, Li et al., Nucleic Acids Research, 28(2): e1 (i-v) (2000); Liu et al., Biochem Cell Bio 80:17-22 (2000); and Burczak et al., Polymorphism Detection and Analysis, Eaton Publishing, 2000; Sheffield et al., Proc. Natl. Acad. Sci. USA, 86:232-236 (1989); Orita et al., Proc. Natl. Acad. Sci. USA, 86:2766-2770 (1989); Flavell et al., Cell, 15:25-41 (1978); Geever et al., Proc. Natl. Acad. Sci. USA, 78:5081-5085 (1981); Cotton et al., Proc. Natl. Acad. Sci. USA, 85:4397-4401 (1985); Myers et al., Science 230:1242-1246 (1985); Church and Gilbert, Proc. Natl. Acad. Sci. USA, 81:1991-1995 (1988); Sanger et al., Proc. Natl. Acad. Sci. USA, 74:5463-5467 (1977); and Beavis et al., U.S. Pat. No. 5,288,644). In a general sense, sequence data establishes the identity of particular nucleotide along a nucleic acid molecule. For polymorphic sites, sequence data established the identity of particular alleles at the polymorphic site. In certain embodiments, sequence data establishes whether particular alleles are present or absent at a polymorphic site.

The sequence data may be obtained from a first sample that is also used to determine PSA values. Alternatively, the sequence data is obtained from a second sample. Nucleic acid sequence data is preferably obtained from a sample that contains nucleic acid, preferably genomic nucleic acid.

Recent technological advances have resulted in technologies that allow massive parallel sequencing, also called high-throughput sequencing, to be performed in relatively condensed format. These technologies share sequencing-by-synthesis principle for generating sequence information, with different technological solutions implemented for extending, tagging and detecting sequences. Exemplary high-throughput sequencing technologies include 454 pyrosequencing technology (Nyren, P. et al. Anal Biochem 208:171-75 (1993); available at 454.com), Illumina Solexa sequencing technology (Bentley, D. R. Curr Opin Genet Dev 16:545-52 (2006); available at illumina.com), and the SOLiD technology developed by Applied Biosystems (ABI) (available at appliedbiosystems.com; see also Strausberg, R. L., et al. Drug Disc Today 13:569-77 (2008)). Other sequencing technologies include those developed by Pacific Biosciences (available at pacificbiosciences.com), Complete Genomics (available at completegenomics.com), Intelligen Bio-Systems (available at intelligentbiosystems.com), Oxford Nanopore Technologies (available at nanoportech.com), Genome Corp (available at genomecorp.com), ION Torrent Systems (available at iontorrent.com) and Helicos Biosciences (available at helicosbio.com). It is contemplated that sequence data useful for performing the present invention may be obtained by any such sequencing method, or other sequencing methods that are developed or made available. Thus, any sequence method that provides the allelic identity at particular polymorphic sites (e.g., the absence or presence of particular alleles at particular polymorphic sites) is useful in the methods described and claimed herein.

Alternatively, determination of the presence or absence of particular alleles can be accomplished using a hybridization method (see Current Protocols in Molecular Biology, Ausubel et al., eds., John Wiley & Sons, including all supplements). A biological sample of genomic DNA, RNA, or cDNA (a “test sample”) is obtained from a test subject or individual suspected of having, being susceptible to, experiencing symptoms associated with, or predisposed for eosinophilia, asthma, and/or myocardial infarction (the “test subject”). The subject can be an adult, child, or fetus. A test sample of DNA from fetal cells or tissue can be obtained by appropriate methods, such as by amniocentesis or chorionic villus sampling. The DNA, RNA, or cDNA sample is then examined. The presence of a specific marker allele can be indicated by sequence-specific hybridization of a nucleic acid probe specific for the particular allele. The presence of more than one specific marker allele or a specific haplotype can be indicated by using several sequence-specific nucleic acid probes, each being specific for a particular allele. In one embodiment, a haplotype can be indicated by a single nucleic acid probe that is specific for the specific haplotype (i.e., hybridizes specifically to a DNA strand comprising the specific marker alleles characteristic of the haplotype). A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample.

To determine whether particular alleles are present at a polymorphic site, a hybridization sample can be formed by contacting the test sample, such as a genomic DNA sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 10, 15, 30, 50, 100, 250 or 500 nucleotides in length that is sufficient to specifically hybridize under stringent conditions to appropriate mRNA or genomic DNA. In certain embodiments, the nucleic acid probe is capable of hybridizing specifically under stringent conditions to a nucleic acid molecule with sequence as set forth in any one of SEQ ID NO: 1-728, or a nucleic acid molecule with the complementary sequence of any one of SEQ ID NO:1-728. Other suitable probes for use in the diagnostic assays of the invention are described herein. Hybridization can be performed by methods well known to the person skilled in the art (see, e.g., Current Protocols in Molecular Biology, Ausubel et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is detected using standard methods. If specific hybridization occurs between the nucleic acid probe and the nucleic acid in the test sample, then the sample contains the allele that is complementary to the nucleotide that is present in the nucleic acid probe. The process can be repeated for any markers of the invention, or markers that make up a haplotype of the invention, or multiple probes can be used concurrently to detect more than one marker alleles at a time.

In certain embodiments, nucleic acid sequence data is obtained by a method that comprises at least one procedure selected from the group consisting of amplification of nucleic acid from a first or second biological sample, hybridization assay using a nucleic acid probe and nucleic acid from the first or second biological sample, and hybridization assay using a nucleic acid probe and nucleic acid obtained by amplification of nucleic acid from the first or second biological sample.

Allele-specific oligonucleotides can also be used to detect the presence of a particular allele in a nucleic acid. An “allele-specific oligonucleotide” (also referred to herein as an “allele-specific oligonucleotide probe”) is an oligonucleotide of approximately 10-50 base pairs or approximately 15-30 base pairs, that specifically hybridizes to a nucleic acid which contains a specific allele at a polymorphic site (e.g., a polymorphic marker as described herein). An allele-specific oligonucleotide probe that is specific for one or more particular alleles at polymorphic markers can be prepared using standard methods (see, e.g., Current Protocols in Molecular Biology, supra). PCR can be used to amplify the desired region. Specific hybridization of an allele-specific oligonucleotide probe to DNA from the subject is indicative of a specific allele at a polymorphic site (see, e.g., Gibbs et al., Nucleic Acids Res. 17:2437-2448 (1989) and WO 93/22456).

In another embodiment, arrays of oligonucleotide probes that are complementary to target nucleic acid sequence segments from a subject, can be used to identify polymorphisms in a nucleic acid. The polymorphism may for example be any one or a combination of rs401681, rs2736098, rs10788160, rs11067228, rs10993994, rs4430796, rs2735839 and rs17632542, and markers in linkage disequilibrium therewith). For example, an oligonucleotide array can be used. Oligonucleotide arrays typically comprise a plurality of different oligonucleotide probes that are coupled to a surface of a substrate in different known locations. These arrays can generally be produced using mechanical synthesis methods or light directed synthesis methods that incorporate a combination of photolithographic methods and solid phase oligonucleotide synthesis methods, or by other methods known to the person skilled in the art (see, e.g., Bier et al., Adv Biochem Eng Biotechnol 109:433-53 (2008); Hoheisel, Nat Rev Genet. 7:200-10 (2006); Fan et al., Methods Enzymol 410:57-73 (2006); Raqoussis & Elvidge, Expert Rev Mol Diagn 6:145-52 (2006); Mockler et al., Genomics 85:1-15 (2005), and references cited therein, the entire teachings of each of which are incorporated by reference herein). Many additional descriptions of the preparation and use of oligonucleotide arrays for detection of polymorphisms can be found, for example, in U.S. Pat. No. 6,858,394, U.S. Pat. No. 6,429,027, U.S. Pat. No. 5,445,934, U.S. Pat. No. 5,700,637, U.S. Pat. No. 5,744,305, U.S. Pat. No. 5,945,334, U.S. Pat. No. 6,054,270, U.S. Pat. No. 6,300,063, U.S. Pat. No. 6,733,977, U.S. Pat. No. 7,364,858, EP 619 321, and EP 373 203, the entire teachings of which are incorporated by reference herein.

Also, standard techniques for genotyping can be used, such as fluorescence-based techniques (e.g., Chen et al., Genome Res. 9(5): 492-98 (1999); Kutyavin et al., Nucleic Acid Res. 34:e128 (2006)), utilizing PCR, LCR, Nested PCR and other techniques for nucleic acid amplification. Specific commercial methodologies available for SNP genotyping include, but are not limited to, TaqMan genotyping assays and SNPlex platforms (Applied Biosystems), gel electrophoresis (Applied Biosystems), mass spectrometry (e.g., MassARRAY system from Sequenom), minisequencing methods, real-time PCR, Bio-Plex system (BioRad), CEQ and SNPstream systems (Beckman), array hybridization technology(e.g., Affymetrix GeneChip; Perlegen), BeadArray Technologies (e.g., Illumina GoldenGate and Infinium assays), array tag technology (e.g., Parallele), and endonuclease-based fluorescence hybridization technology (Invader; Third Wave). Some of the available array platforms, including Affymetrix SNP Array 6.0 and Illumina CNV370-Duo and 1M BeadChips, include SNPs that tag certain copy number variations (CNVs). This allows detection of CNVs via surrogate SNPs included in these platforms. Thus, by use of these or other methods available to the person skilled in the art, one or more alleles at polymorphic markers, including microsatellites, SNPs or other types of polymorphic markers, can be identified.

The direct sequence analysis can be of the nucleic acid of a biological sample obtained from the human individual for which a susceptibility is being determined. The biological sample can be any sample containing nucleic acid (e.g., genomic DNA) obtained from the human individual. For example, the biological sample can be a blood sample, a serum sample, a leukapheresis sample, an amniotic fluid sample, a cerebrospinal fluid sample, a hair sample, a tissue sample from skin, muscle, buccal, or conjuctival mucosa, placenta, gastrointestinal tract, or other organs, a semen sample, a urine sample, a saliva sample, a nail sample, a tooth sample, and the like.

In a specific aspect of the invention, obtaining nucleic acid sequence data comprises obtaining nucleic acid sequence information from a preexisting record, e.g., a preexisting medical record comprising genotype information of the human individual. For example, direct sequence analysis of the allele of the polymorphic marker can be accomplished by mining a pre-existing genotype dataset for the sequence of the allele of the polymorphic marker.

### Indirect Analysis

Alternatively, the nucleic acid sequence data may be obtained through indirect analysis of the nucleic acid sequence of the allele of the polymorphic marker. For example, the allele could be one which leads to the expression of a variant protein comprising an altered amino acid sequence, as compared to the non-variant (e.g., wild-type) protein, due to one or more amino acid substitutions, deletions, or insertions, or truncation (due to, e.g., splice variation). For example, the allele could be the T allele of rs17632542, which leads to a substitution of Isoleucine to Threonine at position 179 of GenBank Accession No. NP_{—}001639. In this instance, nucleic acid sequence data about the allele of the polymorphic marker (e.g., rs17632542) can be obtained through detection of the amino acid substitution of the variant protein. Methods of detecting variant proteins are known in the art. For example, direct amino acid sequencing of the variant protein followed by comparison to a reference amino acid sequence can be used. Also, Immunoassays, e.g., immunofluorescent immunoassays, immunoprecipitations, radioimmunoasays, ELISA, and Western blotting, in which an antibody specific for an epitope comprising the variant sequence among the variant protein and non-variant or wild-type protein can be used.

It is also possible, for example, for the variant protein to demonstrate altered (e.g., upregulated or downregulated) biological activity, in comparison to the non-variant or wild-type protein. The biological activity can be, for example, a binding activity or enzymatic activity. In this instance, nucleic acid sequence data about the allele of the polymorphic marker can be obtained through detection of the altered biological activity. Methods of detecting binding activity and enzymatic activity are known in the art and include, for instance, ELISA, competitive binding assays, quantitative binding assays using instruments such as, for example, a Biacore® 3000 instrument, chromatographic assays, e.g., HPLC and TLC.

Alternatively or additionally, the polymorphic variant (the allele of the polymorphic marker) could lead to an altered expression level, e.g., an increased expression level of an mRNA or protein, a decreased expression level of an mRNA or protein. Nucleic acid sequence data about the allele of the polymorphic marker can, in these instances, be obtained through detection of the altered expression level. Methods of detecting expression levels are known in the art. For example, ELISA, radioimmunoassays, immunofluorescence, and Western blotting can be used to compare the expression of protein levels. Alternatively, Northern blotting can be used to compare the levels of mRNA. These processes are described in Sambrook et al., Molecular Cloning: A Laboratory Manual, 3^{rd} ed. Cold Spring Harbor Laboratory Press, Cold Spring Harbor, N.Y. (2001).

The indirect sequence analysis can be of a nucleic acid (e.g., DNA, mRNA) or protein of a biological sample obtained from the human individual for which a susceptibility is being determined. The biological sample can be any nucleic acid or protein containing sample obtained from the human individual. For example, the biological sample can be any of the biological samples described herein.

In view of the foregoing, analyzing the sequence of at least one polymorphic marker can comprise determining the presence or absence of at least one allele of the marker. Alternatively, the analyzing can comprise analyzing the sequence of the polymorphic marker in a particular sample. Further, analyzing the sequence of the at least one polymorphic marker can comprise determining the presence or absence of an amino acid substitution in the amino acid sequence encoded by the polymorphic marker, or it can comprise obtaining a biological sample from the human individual and analyzing the amino acid sequence encoded by at least one gene of the group. In certain embodiments, analyzing sequence comprises determining the identity of both alleles of the at least one polymorphic marker. Such sequence analysis thus corresponds to establishing the genotype of a particular marker for an individual.

### Linkage Disequilibrium

The nucleic acid sequence data may be obtained through other means of indirect analysis of the nucleic acid sequence of the allele of the polymorphic marker. For example, obtaining nucleic acid data can comprise identifying at least one allele of a marker in linkage disequilibrium with at least one polymorphic marker associated with PSA levels. Linkage Disequilibrium (LD) refers to a non-random assortment of two genetic elements. For example, if a particular genetic element (e.g., an allele of a polymorphic marker, or a haplotype) occurs in a population at a frequency of 0.50 (50%) and another element occurs at a frequency of 0.50 (50%), then the predicted occurrance of a person's having both elements is 0.25 (25%), assuming a random distribution of the elements. However, if it is discovered that the two elements occur together at a frequency higher than 0.25, then the elements are said to be in linkage disequilibrium, since they tend to be inherited together at a higher rate than what their independent frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the frequency of the occurence of each allele or haplotype in the population. For populations of diploids, e.g., human populations, individuals will typically have two alleles for each genetic element (e.g., a marker, haplotype or gene).

Many different measures have been proposed for assessing the strength of linkage disequilibrium (LD; reviewed in Devlin, B. & Risch, N., Genomics 29:311-22 (1995)). Most capture the strength of association between pairs of biallelic sites. Two important pairwise measures of LD are r² (sometimes denoted Δ²) and |D′| (Lewontin, R., Genetics 49:49-67 (1964); Hill, W. G. & Robertson, A. Theor. Appl. Genet. 22:226-231 (1968)). Both measures range from 0 (no disequilibrium) to 1 (‘complete’ disequilibrium), but their interpretation is slightly different. |D′| is defined in such a way that it is equal to 1 if just two or three of the possible haplotypes are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots.

For the methods described herein, a significant r² value between markers can be at least 0.1 such as at least 0.1, 0.15, 0.2, 0.25, 0.3, 0.35, 0.4, 0.45, 0.5, 0.55, 0.6, 0.65, 0.7, 0.75, 0.8, 0.85, 0.9, 0.91, 0.92, 0.93, 0.94, 0.95, 0.96, 0.97, 0.98, 0.99 or 1.0. In one specific embodiment of invention, the significant r² value can be at least 0.2. This means that markers are considered to be in LD if the correlation coefficient r² between the markers has a value of least 0.2. Alternatively, linkage disequilibrium as described herein, refers to linkage disequilibrium characterized by values of |D′| of at least 0.2, such as 0.3, 0.4, 0.5, 0.6, 0.7, 0.8, 0.85, 0.9, 0.95, 0.96, 0.97, 0.98, 0.99. Thus, linkage disequilibrium represents a correlation between alleles of distinct markers. It is measured by correlation coefficient or |D′| (r² up to 1.0 and |D′| up to 1.0). Linkage disequilibrium can be determined in a single human population, as defined herein, or it can be determined in a collection of samples comprising individuals from more than one human population. In one embodiment of the invention, LD is determined in a sample from one or more of the HapMap populations. These include samples from the Yoruba people of Ibadan, Nigeria (YRI), samples from individuals from the Tokyo area in Japan (JPT), samples from individuals Beijing, China (CHB), and samples from U.S. residents with northern and western European ancestry (CEU), as described (The International HapMap Consortium, Nature 426:789-796 (2003)). In one such embodiment, LD is determined in the Caucasian CEU population of the HapMap samples. In yet another embodiment, LD is determined in samples from the Icelandic population. In another embodiment, LD is determined in samples from the UK population.

If all polymorphisms in the genome were independent at the population level (i.e., no LD between polymorphisms), then every single one of them would need to be investigated in association studies, to assess all different polymorphic states. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

Genomic LD maps have been generated across the genome, and such LD maps have been proposed to serve as framework for mapping disease-genes (Risch, N. & Merkiangas, K, Science 273:1516-1517 (1996); Maniatis, N., et al., Proc Natl Acad Sci USA 99:2228-2233 (2002); Reich, D E et al, Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

There are two main methods for defining these haplotype blocks: blocks can be defined as regions of DNA that have limited haplotype diversity (see, e.g., Daly, M. et al., Nature Genet. 29:229-232 (2001); Patil, N. et al., Science 294:1719-1723 (2001); Dawson, E. et al., Nature 418:544-548 (2002); Zhang, K. et al., Proc. Natl. Acad. Sci. USA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). More recently, a fine-scale map of recombination rates and corresponding hotspots across the human genome has been generated (Myers, S., et al., Science 310:321-32324 (2005); Myers, S. et al., Biochem Soc Trans 34:526530 (2006)). The map reveals the enormous variation in recombination across the genome, with recombination rates as high as 10-60 cM/Mb in hotspots, while closer to 0 in intervening regions, which thus represent regions of limited haplotype diversity and high LD. The map can therefore be used to define haplotype blocks/LD blocks as regions flanked by recombination hotspots. As used herein, the terms “haplotype block” or “LD block” includes blocks defined by any of the above described characteristics, or other alternative methods used by the person skilled in the art to define such regions.

Haplotype blocks (LD blocks) can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

It has thus become apparent that for any given observed association to a polymorphic marker in the genome, it is likely that additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given disease or trait, and as such are useful for use in the methods and kits of the invention. One or more causative (functional) variants or mutations may reside within the region found to be associating to the disease or trait. The functional variant may be another SNP, a tandem repeat polymorphism (such as a minisatellite or a microsatellite), a transposable element, or a copy number variation, such as an inversion, deletion or insertion. Such variants in LD with other variants used to detect an association to a disease or trait (e.g., the variants described herein to be associated with risk of eosinophilia, asthma, myocardial infarction, and/or hypertension) may confer a higher relative risk (RR) or odds ratio (OR) than observed for the tagging markers used to detect the association. The invention thus refers to the markers used for detecting association to the disease, as described herein, as well as markers in linkage disequilibrium with the markers. Thus, in certain embodiments of the invention, markers that are in LD with the markers and/or haplotypes of the invention, as described herein, may be used as surrogate markers. The surrogate markers have in one embodiment relative risk (RR) and/or odds ratio (OR) values smaller than for the markers or haplotypes initially found to be associating with the disease, as described herein. In other embodiments, the surrogate markers have RR or OR values greater than those initially determined for the markers initially found to be associating with the disease, as described herein. An example of such an embodiment would be a rare, or relatively rare (<10% allelic population frequency) variant in LD with a more common variant (>10% population frequency) initially found to be associating with the disease, such as the variants described herein. Identifying and using such markers for detecting the association discovered by the inventors as described herein can be performed by routine methods well known to the person skilled in the art, and are therefore within the scope of the invention.

In view of the foregoing, the marker in linkage disequilibrium with a polymorphic marker associated with PSA levels may be one of the surrogate markers listed in Table 1. The markers were selected using data for Caucasian CEU samples from the 1000 Genomes Project (available at 1000 genomes.org) and the HapMap dataset (available at hapmap.org).

TABLE 1
Surrogate markers for the markers shown herein to be associated with PSA levels.

| | | | | | | | Seq ID |
|---|---|---|---|---|---|---|---|
| | | | Dec. | Inc. | | | NO of |
| Anchor SNP | Surrogate | Position | Allele | Allele | D′ | r² | surrogate |
| rs10788160_1 | s.122837469 | 10-122837469 | C | A | 1 | 0.21 | 305 |
| rs10788160_1 | rs2130779 | 10-122869722 | G | T | 0.73 | 0.21 | 130 |
| rs10788160_1 | s.122876448 | 10-122876448 | G | A | 0.78 | 0.29 | 306 |
| rs10788160_1 | s.122901140 | 10-122901140 | C | T | 1 | 0.28 | 307 |
| rs10788160_1 | s.122901142 | 10-122901142 | A | C | 1 | 0.28 | 308 |
| rs10788160_1 | s.122905335 | 10-122905335 | G | A | 0.71 | 0.29 | 309 |
| rs10788160_1 | rs10788149 | 10-122957160 | A | G | 0.59 | 0.24 | 24 |
| rs10788160_1 | rs10749408 | 10-122957516 | T | C | 0.79 | 0.37 | 15 |
| rs10788160_1 | rs2172071 | 10-122958020 | T | C | 0.65 | 0.28 | 131 |
| rs10788160_1 | rs11592107 | 10-122958954 | G | A | 0.59 | 0.24 | 89 |
| rs10788160_1 | rs1907218 | 10-122960206 | C | T | 0.65 | 0.28 | 122 |
| rs10788160_1 | rs1907220 | 10-122960913 | G | A | 0.65 | 0.28 | 123 |
| rs10788160_1 | rs1994655 | 10-122961236 | G | T | 0.65 | 0.28 | 127 |
| rs10788160_1 | rs1907221 | 10-122962417 | T | C | 0.59 | 0.24 | 124 |
| rs10788160_1 | rs1907225 | 10-122965623 | T | C | 0.65 | 0.28 | 125 |
| rs10788160_1 | rs1907226 | 10-122965736 | A | G | 0.65 | 0.28 | 126 |
| rs10788160_1 | rs10749409 | 10-122966556 | G | C | 0.65 | 0.28 | 16 |
| rs10788160_1 | rs11199835 | 10-122967147 | A | G | 0.65 | 0.28 | 66 |
| rs10788160_1 | s.122991926 | 10-122991926 | T | C | 0.74 | 0.25 | 310 |
| rs10788160_1 | rs729014 | 10-122992796 | C | T | 0.88 | 0.34 | 274 |
| rs10788160_1 | s.122993518 | 10-122993518 | A | G | 0.83 | 0.66 | 311 |
| rs10788160_1 | s.122994309 | 10-122994309 | G | A | 0.83 | 0.66 | 312 |
| rs10788160_1 | s.122994946 | 10-122994946 | T | G | 1 | 0.25 | 313 |
| rs10788160_1 | rs1873450 | 10-122996264 | T | G | 0.84 | 0.7 | 116 |
| rs10788160_1 | rs2901290 | 10-122997016 | G | A | 0.8 | 0.42 | 167 |
| rs10788160_1 | s.122998594 | 10-122998594 | G | A | 0.8 | 0.42 | 314 |
| rs10788160_1 | s.122998678 | 10-122998678 | G | T | 1 | 0.21 | 315 |
| rs10788160_1 | s.122998978 | 10-122998978 | A | T | 0.75 | 0.27 | 316 |
| rs10788160_1 | rs2201026 | 10-122998993 | T | G | 0.86 | 0.47 | 132 |
| rs10788160_1 | rs4237529 | 10-122999123 | A | G | 0.8 | 0.42 | 200 |
| rs10788160_1 | s.122999386 | 10-122999386 | A | G | 0.84 | 0.7 | 317 |
| rs10788160_1 | rs1873451 | 10-123000467 | T | C | 0.8 | 0.42 | 117 |
| rs10788160_1 | rs1873452 | 10-123000564 | T | C | 0.8 | 0.42 | 118 |
| rs10788160_1 | rs4752520 | 10-123001514 | C | T | 0.8 | 0.42 | 230 |
| rs10788160_1 | rs10886880 | 10-123003911 | T | C | 0.84 | 0.7 | 37 |
| rs10788160_1 | rs10749412 | 10-123007551 | A | T | 0.8 | 0.42 | 17 |
| rs10788160_1 | s.123008216 | 10-123008216 | G | A | 0.8 | 0.42 | 318 |
| rs10788160_1 | rs3925042 | 10-123009010 | C | T | 0.8 | 0.42 | 191 |
| rs10788160_1 | rs1125527 | 10-123009606 | G | A | 0.8 | 0.42 | 85 |
| rs10788160_1 | rs1125528 | 10-123009942 | T | A | 0.84 | 0.7 | 86 |
| rs10788160_1 | rs4319451 | 10-123010241 | A | G | 1 | 0.21 | 205 |
| rs10788160_1 | rs10788154 | 10-123011231 | A | C | 0.8 | 0.42 | 25 |
| rs10788160_1 | rs7081844 | 10-123011258 | C | T | 0.8 | 0.42 | 265 |
| rs10788160_1 | rs7076500 | 10-123011721 | G | A | 0.8 | 0.44 | 262 |
| rs10788160_1 | s.123011774 | 10-123011774 | C | T | 0.8 | 0.42 | 319 |
| rs10788160_1 | s.123011879 | 10-123011879 | C | T | 0.8 | 0.42 | 320 |
| rs10788160_1 | rs11199862 | 10-123012946 | G | A | 0.84 | 0.7 | 67 |
| rs10788160_1 | s.123014171 | 10-123014171 | T | C | 0.77 | 0.41 | 321 |
| rs10788160_1 | rs12146156 | 10-123014406 | T | C | 0.94 | 0.84 | 99 |
| rs10788160_1 | s.123014499 | 10-123014499 | A | G | 0.94 | 0.84 | 322 |
| rs10788160_1 | s.123014519 | 10-123014519 | G | A | 0.89 | 0.38 | 323 |
| rs10788160_1 | rs12146366 | 10-123014670 | C | T | 0.94 | 0.84 | 100 |
| rs10788160_1 | s.123014684 | 10-123014684 | C | A | 0.87 | 0.52 | 324 |
| rs10788160_1 | rs7091083 | 10-123014747 | G | A | 0.87 | 0.52 | 269 |
| rs10788160_1 | rs7074985 | 10-123014878 | T | A | 0.87 | 0.52 | 259 |
| rs10788160_1 | rs7915008 | 10-123015215 | G | A | 0.94 | 0.79 | 285 |
| rs10788160_1 | s.123015342 | 10-123015342 | C | A | 1 | 0.3 | 325 |
| rs10788160_1 | s.123015365 | 10-123015365 | G | A | 0.87 | 0.52 | 326 |
| rs10788160_1 | rs10749413 | 10-123015655 | A | T | 0.87 | 0.52 | 18 |
| rs10788160_1 | rs11199866 | 10-123015727 | G | A | 0.87 | 0.52 | 68 |
| rs10788160_1 | s.123016003 | 10-123016003 | G | A | 0.94 | 0.84 | 327 |
| rs10788160_1 | rs7923130 | 10-123016492 | G | A | 0.87 | 0.52 | 288 |
| rs10788160_1 | rs7922901 | 10-123016509 | C | G | 0.87 | 0.52 | 287 |
| rs10788160_1 | rs10886882 | 10-123017023 | C | T | 0.87 | 0.52 | 38 |
| rs10788160_1 | rs10886883 | 10-123017171 | C | G | 0.87 | 0.52 | 39 |
| rs10788160_1 | rs11199867 | 10-123017394 | G | T | 0.87 | 0.52 | 69 |
| rs10788160_1 | s.123017698 | 10-123017698 | C | T | 1 | 0.44 | 328 |
| rs10788160_1 | s.123018111 | 10-123018111 | G | C | 0.87 | 0.52 | 329 |
| rs10788160_1 | rs4393247 | 10-123018166 | G | A | 0.94 | 0.84 | 206 |
| rs10788160_1 | s.123018188 | 10-123018188 | C | T | 0.87 | 0.52 | 330 |
| rs10788160_1 | rs4489674 | 10-123018240 | A | G | 0.87 | 0.52 | 210 |
| rs10788160_1 | rs11199868 | 10-123018329 | T | A | 0.94 | 0.84 | 70 |
| rs10788160_1 | s.123018670 | 10-123018670 | G | T | 0.94 | 0.84 | 331 |
| rs10788160_1 | s.123019408 | 10-123019408 | T | G | 0.87 | 0.49 | 332 |
| rs10788160_1 | s.123019759 | 10-123019759 | C | G | 0.87 | 0.52 | 333 |
| rs10788160_1 | rs11199869 | 10-123020055 | A | G | 0.94 | 0.84 | 71 |
| rs10788160_1 | s.123020245 | 10-123020245 | G | T | 1 | 0.44 | 334 |
| rs10788160_1 | s.123020365 | 10-123020365 | A | T | 0.87 | 0.52 | 335 |
| rs10788160_1 | rs10886885 | 10-123020471 | G | T | 0.94 | 0.84 | 40 |
| rs10788160_1 | rs10788159 | 10-123020775 | A | G | 0.94 | 0.84 | 26 |
| rs10788160_1 | rs10886886 | 10-123020859 | T | G | 0.94 | 0.79 | 41 |
| rs10788160_1 | rs11199871 | 10-123020940 | C | A | 0.94 | 0.74 | 72 |
| rs10788160_1 | rs11199872 | 10-123021180 | G | A | 0.94 | 0.84 | 73 |
| rs10788160_1 | rs12761612 | 10-123021400 | G | A | 0.94 | 0.84 | 106 |
| rs10788160_1 | rs4575197 | 10-123022158 | A | G | 1 | 0.3 | 220 |
| rs10788160_1 | rs11199874 | 10-123022509 | G | A | 1 | 0.95 | 74 |
| rs10788160_1 | rs10886887 | 10-123023168 | C | T | 1 | 1 | 42 |
| rs10788160_1 | s.123023625 | 10-123023625 | G | T | 1 | 0.95 | 336 |
| rs10788160_1 | s.123023836 | 10-123023836 | T | C | 1 | 0.95 | 337 |
| rs10788160_1 | rs4465316 | 10-123024171 | C | A | 1 | 0.95 | 207 |
| rs10788160_1 | rs4468286 | 10-123024381 | C | A | 1 | 0.95 | 208 |
| rs10788160_1 | rs10886890 | 10-123027193 | A | G | 1 | 0.95 | 43 |
| rs10788160_1 | rs10788162 | 10-123027299 | A | G | 1 | 0.6 | 27 |
| rs10788160_1 | s.123028135 | 10-123028135 | C | A | 1 | 1 | 338 |
| rs10788160_1 | rs12413648 | 10-123028887 | G | A | 1 | 1 | 103 |
| rs10788160_1 | s.123029102 | 10-123029102 | T | C | 1 | 1 | 339 |
| rs10788160_1 | rs10788163 | 10-123029792 | T | G | 1 | 1 | 28 |
| rs10788160_1 | s.123031617 | 10-123031617 | G | T | 1 | 1 | 340 |
| rs10788160_1 | s.123031811 | 10-123031811 | A | T | 1 | 1 | 341 |
| rs10788160_1 | rs10788164 | 10-123032835 | C | T | 1 | 0.63 | 29 |
| rs10788160_1 | rs11598592 | 10-123033379 | G | A | 1 | 0.47 | 91 |
| rs10788160_1 | rs10788165 | 10-123034204 | T | G | 1 | 0.63 | 30 |
| rs10788160_1 | rs9630106 | 10-123034373 | A | G | 1 | 0.47 | 292 |
| rs10788160_1 | rs10886893 | 10-123034442 | T | C | 1 | 0.95 | 44 |
| rs10788160_1 | s.123034821 | 10-123034821 | T | C | 0.95 | 0.9 | 342 |
| rs10788160_1 | rs11199879 | 10-123035202 | T | C | 0.95 | 0.9 | 75 |
| rs10788160_1 | rs11199881 | 10-123035860 | T | C | 1 | 0.95 | 76 |
| rs10788160_1 | rs12415826 | 10-123036368 | T | C | 1 | 0.95 | 104 |
| rs10788160_1 | rs10788166 | 10-123036532 | A | G | 1 | 0.95 | 31 |
| rs10788160_1 | rs10886894 | 10-123036863 | T | C | 1 | 0.95 | 45 |
| rs10788160_1 | rs10886895 | 10-123037303 | C | A | 1 | 0.95 | 46 |
| rs10788160_1 | rs10886896 | 10-123037386 | C | A | 1 | 0.95 | 47 |
| rs10788160_1 | rs10886897 | 10-123037630 | T | C | 1 | 0.95 | 48 |
| rs10788160_1 | rs10886898 | 10-123037681 | T | G | 1 | 0.95 | 49 |
| rs10788160_1 | rs10886899 | 10-123037711 | G | T | 1 | 0.95 | 50 |
| rs10788160_1 | rs10886900 | 10-123037998 | A | G | 1 | 0.95 | 51 |
| rs10788160_1 | rs10886901 | 10-123038120 | T | C | 1 | 0.95 | 52 |
| rs10788160_1 | rs10886902 | 10-123039254 | T | C | 1 | 0.95 | 53 |
| rs10788160_1 | rs10886903 | 10-123039425 | C | G | 1 | 0.95 | 54 |
| rs10788160_1 | rs12413088 | 10-123042718 | C | T | 1 | 0.95 | 102 |
| rs10788160_1 | rs10788167 | 10-123044008 | T | A | 1 | 0.95 | 32 |
| rs10788160_1 | s.123047182 | 10-123047182 | C | T | 1 | 0.28 | 343 |
| rs10788160_1 | rs7085073 | 10-123047258 | C | T | 1 | 0.28 | 266 |
| rs10788160_1 | rs7071101 | 10-123047771 | G | A | 1 | 0.28 | 257 |
| rs10788160_1 | rs12570783 | 10-123049889 | G | A | 1 | 0.28 | 105 |
| rs10788160_1 | rs11199884 | 10-123053164 | G | A | 0.75 | 0.37 | 77 |
| rs10788160_1 | rs7085506 | 10-123054129 | C | G | 1 | 0.28 | 267 |
| rs10788160_1 | rs10886905 | 10-123057992 | T | C | 0.82 | 0.41 | 55 |
| rs10788160_1 | rs10736302 | 10-123059707 | T | C | 0.75 | 0.37 | 14 |
| rs10788160_1 | s.123061811 | 10-123061811 | C | T | 1 | 0.28 | 344 |
| rs10788160_1 | s.123062031 | 10-123062031 | G | C | 1 | 0.28 | 345 |
| rs10788160_1 | rs11199886 | 10-123062077 | G | T | 0.75 | 0.37 | 78 |
| rs10788160_1 | s.123063327 | 10-123063327 | A | T | 1 | 0.28 | 346 |
| rs10788160_1 | s.123063715 | 10-123063715 | G | A | 0.75 | 0.37 | 347 |
| rs10788160_1 | rs10886907 | 10-123063722 | G | C | 0.75 | 0.37 | 56 |
| rs10788160_1 | s.123064252 | 10-123064252 | C | T | 0.81 | 0.37 | 348 |
| rs10788160_1 | s.123064345 | 10-123064345 | G | T | 0.75 | 0.37 | 349 |
| rs10788160_1 | s.123064780 | 10-123064780 | C | T | 0.82 | 0.41 | 350 |
| rs10788160_1 | s.123064783 | 10-123064783 | T | C | 0.75 | 0.37 | 351 |
| rs10788160_1 | s.123066424 | 10-123066424 | T | C | 0.75 | 0.37 | 352 |
| rs10788160_1 | s.123066700 | 10-123066700 | T | C | 0.75 | 0.37 | 353 |
| rs10788160_1 | rs3981043 | 10-123066817 | A | T | 1 | 0.26 | 192 |
| rs10788160_1 | rs11199896 | 10-123067415 | C | T | 0.81 | 0.37 | 79 |
| rs10788160_1 | rs11199897 | 10-123067723 | G | A | 0.75 | 0.37 | 80 |
| rs10788160_1 | rs11199898 | 10-123067775 | T | C | 0.82 | 0.41 | 81 |
| rs10788160_1 | s.123067963 | 10-123067963 | T | A | 0.75 | 0.37 | 354 |
| rs10788160_1 | rs11199900 | 10-123067986 | A | T | 0.75 | 0.37 | 82 |
| rs10788160_1 | rs11199901 | 10-123068059 | C | T | 0.75 | 0.37 | 83 |
| rs10788160_1 | s.123068178 | 10-123068178 | G | T | 0.73 | 0.33 | 355 |
| rs10788160_1 | s.123068222 | 10-123068222 | G | A | 0.75 | 0.37 | 356 |
| rs10788160_1 | s.123068236 | 10-123068236 | C | T | 0.9 | 0.42 | 357 |
| rs10788160_1 | s.123068424 | 10-123068424 | A | G | 0.73 | 0.33 | 358 |
| rs10788160_1 | s.123068619 | 10-123068619 | C | T | 0.82 | 0.41 | 359 |
| rs10788160_1 | s.123068743 | 10-123068743 | A | G | 0.9 | 0.42 | 360 |
| rs10788160_1 | s.123068926 | 10-123068926 | A | T | 1 | 0.44 | 361 |
| rs10788160_1 | s.123068997 | 10-123068997 | G | A | 0.73 | 0.33 | 362 |
| rs10788160_1 | s.123069012 | 10-123069012 | C | T | 1 | 0.27 | 363 |
| rs10788160_1 | s.123069326 | 10-123069326 | G | T | 0.88 | 0.34 | 364 |
| rs10788160_1 | s.123069570 | 10-123069570 | C | T | 0.81 | 0.37 | 365 |
| rs10788160_1 | s.123069989 | 10-123069989 | T | C | 0.75 | 0.37 | 366 |
| rs10788160_1 | s.123070105 | 10-123070105 | C | T | 0.73 | 0.33 | 367 |
| rs10788160_1 | s.123071090 | 10-123071090 | G | A | 0.75 | 0.37 | 368 |
| rs10788160_1 | s.123071347 | 10-123071347 | G | C | 1 | 0.26 | 369 |
| rs10788160_1 | rs4254007 | 10-123071380 | T | A | 1 | 0.27 | 202 |
| rs10788160_1 | s.123071495 | 10-123071495 | G | A | 1 | 0.27 | 370 |
| rs10788160_1 | s.123071914 | 10-123071914 | G | T | 1 | 0.36 | 371 |
| rs10788160_1 | s.123072804 | 10-123072804 | G | A | 1 | 0.48 | 372 |
| rs10788160_1 | rs7900630 | 10-123073094 | C | T | 1 | 0.27 | 283 |
| rs10788160_1 | s.123074016 | 10-123074016 | T | C | 0.57 | 0.26 | 373 |
| rs10788160_1 | rs1896416 | 10-123074480 | G | A | 0.57 | 0.26 | 119 |
| rs10788160_1 | s.123074531 | 10-123074531 | C | T | 0.88 | 0.34 | 374 |
| rs10788160_1 | s.123074928 | 10-123074928 | C | T | 0.75 | 0.37 | 375 |
| rs10788160_1 | s.123076274 | 10-123076274 | T | C | 1 | 0.65 | 376 |
| rs10788160_1 | s.123076472 | 10-123076472 | C | G | 1 | 0.27 | 377 |
| rs10788160_1 | rs2420925 | 10-123077176 | T | C | 1 | 0.27 | 135 |
| rs10788160_1 | s.123077398 | 10-123077398 | A | G | 1 | 0.27 | 378 |
| rs10788160_1 | s.123077455 | 10-123077455 | G | C | 1 | 0.27 | 379 |
| rs10788160_1 | rs12779205 | 10-123077742 | A | T | 1 | 0.65 | 108 |
| rs10788160_1 | rs11199912 | 10-123078010 | G | T | 1 | 0.27 | 84 |
| rs10788160_1 | rs4752534 | 10-123078189 | T | C | 1 | 0.24 | 231 |
| rs10788160_1 | s.123078389 | 10-123078389 | A | T | 1 | 0.28 | 380 |
| rs10788160_1 | rs1896420 | 10-123078843 | C | T | 1 | 0.28 | 121 |
| rs10788160_1 | rs1896419 | 10-123079069 | A | C | 1 | 0.23 | 120 |
| rs10788160_1 | s.123079199 | 10-123079199 | G | A | 1 | 0.28 | 381 |
| rs10788160_1 | s.123081990 | 10-123081990 | T | A | 1 | 0.21 | 382 |
| rs10788160_1 | s.123081993 | 10-123081993 | T | A | 1 | 0.25 | 383 |
| rs10788160_1 | s.123081998 | 10-123081998 | A | G | 1 | 0.32 | 384 |
| rs10788160_1 | s.123201870 | 10-123201870 | T | C | 1 | 0.21 | 385 |
| rs10993994_4 | s.51157005 | 10-51157005 | A | G | 0.8 | 0.48 | 459 |
| rs10993994_4 | s.51159221 | 10-51159221 | T | C | 0.8 | 0.48 | 460 |
| rs10993994_4 | rs35716372 | 10-51159230 | G | A | 0.65 | 0.27 | 177 |
| rs10993994_4 | s.51159373 | 10-51159373 | T | C | 0.8 | 0.48 | 461 |
| rs10993994_4 | s.51159376 | 10-51159376 | G | C | 0.8 | 0.48 | 462 |
| rs10993994_4 | s.51159399 | 10-51159399 | G | T | 0.8 | 0.48 | 463 |
| rs10993994_4 | s.51159786 | 10-51159786 | G | C | 0.8 | 0.48 | 464 |
| rs10993994_4 | rs4935090 | 10-51161131 | A | T | 0.8 | 0.48 | 232 |
| rs10993994_4 | rs12781411 | 10-51161595 | C | T | 0.8 | 0.48 | 109 |
| rs10993994_4 | s.51162137 | 10-51162137 | A | G | 0.8 | 0.48 | 465 |
| rs10993994_4 | s.51162792 | 10-51162792 | C | A | 0.8 | 0.48 | 466 |
| rs10993994_4 | s.51162795 | 10-51162795 | C | A | 0.8 | 0.48 | 467 |
| rs10993994_4 | rs11004246 | 10-51165355 | T | C | 0.8 | 0.48 | 58 |
| rs10993994_4 | s.51165690 | 10-51165690 | A | C | 0.79 | 0.44 | 468 |
| rs10993994_4 | rs11004324 | 10-51166629 | T | G | 0.8 | 0.48 | 59 |
| rs10993994_4 | rs2843562 | 10-51166802 | T | C | 0.8 | 0.51 | 165 |
| rs10993994_4 | rs11004409 | 10-51168025 | G | C | 0.95 | 0.61 | 60 |
| rs10993994_4 | rs11004415 | 10-51168187 | G | A | 1 | 0.61 | 61 |
| rs10993994_4 | rs11004422 | 10-51168342 | A | G | 0.65 | 0.35 | 62 |
| rs10993994_4 | s.51168415 | 10-51168415 | C | T | 0.63 | 0.28 | 469 |
| rs10993994_4 | rs11004435 | 10-51168499 | C | A | 0.65 | 0.35 | 63 |
| rs10993994_4 | rs11599333 | 10-51169661 | A | C | 1 | 0.61 | 92 |
| rs10993994_4 | s.51170094 | 10-51170094 | T | G | 1 | 0.61 | 470 |
| rs10993994_4 | s.51170307 | 10-51170307 | G | A | 1 | 0.61 | 471 |
| rs10993994_4 | rs12763717 | 10-51170880 | C | G | 1 | 0.61 | 107 |
| rs10993994_4 | rs67289834 | 10-51171310 | C | T | 1 | 0.65 | 251 |
| rs10993994_4 | s.51172442 | 10-51172442 | T | A | 1 | 0.61 | 472 |
| rs10993994_4 | s.51172558 | 10-51172558 | T | G | 1 | 0.61 | 473 |
| rs10993994_4 | rs57858801 | 10-51172580 | A | T | 1 | 0.61 | 244 |
| rs10993994_4 | s.51172618 | 10-51172618 | C | A | 1 | 0.61 | 474 |
| rs10993994_4 | s.51172808 | 10-51172808 | C | G | 1 | 0.61 | 475 |
| rs10993994_4 | s.51173184 | 10-51173184 | A | G | 1 | 0.61 | 476 |
| rs10993994_4 | rs7071471 | 10-51173341 | C | T | 1 | 0.61 | 258 |
| rs10993994_4 | rs7090326 | 10-51173381 | A | T | 1 | 0.61 | 268 |
| rs10993994_4 | s.51173565 | 10-51173565 | C | G | 1 | 0.61 | 477 |
| rs10993994_4 | s.51173983 | 10-51173983 | T | C | 1 | 0.61 | 478 |
| rs10993994_4 | s.51174391 | 10-51174391 | A | G | 1 | 0.61 | 479 |
| rs10993994_4 | s.51174499 | 10-51174499 | A | C | 0.86 | 0.63 | 480 |
| rs10993994_4 | s.51174610 | 10-51174610 | C | T | 0.86 | 0.63 | 481 |
| rs10993994_4 | s.51174944 | 10-51174944 | G | A | 1 | 0.61 | 482 |
| rs10993994_4 | s.51175013 | 10-51175013 | G | A | 0.73 | 0.34 | 483 |
| rs10993994_4 | s.51175409 | 10-51175409 | A | G | 1 | 0.61 | 484 |
| rs10993994_4 | s.51176290 | 10-51176290 | C | T | 1 | 0.61 | 485 |
| rs10993994_4 | s.51176963 | 10-51176963 | T | C | 1 | 0.61 | 486 |
| rs10993994_4 | s.51180209 | 10-51180209 | G | A | 1 | 0.7 | 487 |
| rs10993994_4 | rs10825652 | 10-51180767 | G | A | 1 | 0.7 | 33 |
| rs10993994_4 | s.51180819 | 10-51180819 | C | A | 1 | 0.7 | 488 |
| rs10993994_4 | rs2843560 | 10-51182135 | C | G | 1 | 0.61 | 164 |
| rs10993994_4 | rs2125770 | 10-51184830 | C | T | 1 | 0.61 | 129 |
| rs10993994_4 | rs2611513 | 10-51185463 | T | C | 1 | 0.7 | 144 |
| rs10993994_4 | rs2611512 | 10-51185540 | G | A | 1 | 0.61 | 143 |
| rs10993994_4 | rs2611509 | 10-51186258 | A | G | 1 | 0.7 | 142 |
| rs10993994_4 | s.51186305 | 10-51186305 | T | G | 1 | 0.7 | 489 |
| rs10993994_4 | rs2926494 | 10-51187362 | C | T | 1 | 0.7 | 168 |
| rs10993994_4 | rs2611508 | 10-51188053 | A | T | 1 | 0.7 | 141 |
| rs10993994_4 | rs2611507 | 10-51188679 | C | T | 0.95 | 0.69 | 140 |
| rs10993994_4 | s.51188694 | 10-51188694 | C | A | 1 | 0.7 | 490 |
| rs10993994_4 | rs2611506 | 10-51188793 | T | C | 1 | 0.7 | 139 |
| rs10993994_4 | rs57263518 | 10-51189160 | G | A | 1 | 0.7 | 243 |
| rs10993994_4 | s.51189522 | 10-51189522 | A | G | 0.95 | 0.69 | 491 |
| rs10993994_4 | rs3101227 | 10-51190209 | A | C | 1 | 0.7 | 170 |
| rs10993994_4 | rs2843549 | 10-51191253 | A | C | 1 | 0.7 | 160 |
| rs10993994_4 | rs2843550 | 10-51191458 | T | C | 1 | 0.7 | 161 |
| rs10993994_4 | rs2249986 | 10-51191690 | G | T | 1 | 0.7 | 133 |
| rs10993994_4 | rs2843551 | 10-51191951 | A | C | 1 | 0.7 | 162 |
| rs10993994_4 | s.51192126 | 10-51192126 | T | C | 0.95 | 0.69 | 492 |
| rs10993994_4 | rs7077830 | 10-51192282 | C | G | 0.95 | 0.69 | 263 |
| rs10993994_4 | s.51193219 | 10-51193219 | T | A | 1 | 0.73 | 493 |
| rs10993994_4 | rs2843554 | 10-51193867 | T | G | 1 | 0.73 | 163 |
| rs10993994_4 | s.51194280 | 10-51194280 | T | C | 1 | 0.31 | 494 |
| rs10993994_4 | rs2611489 | 10-51194895 | A | G | 1 | 0.73 | 138 |
| rs10993994_4 | rs3123078 | 10-51194977 | T | C | 1 | 0.73 | 171 |
| rs10993994_4 | rs4935162 | 10-51195705 | C | G | 1 | 0.73 | 233 |
| rs10993994_4 | rs7081532 | 10-51196099 | G | A | 1 | 0.7 | 264 |
| rs10993994_4 | rs10826075 | 10-51197376 | C | G | 0.74 | 0.54 | 34 |
| rs10993994_4 | rs7896156 | 10-51199385 | G | A | 1 | 0.7 | 282 |
| rs10993994_4 | s.51199599 | 10-51199599 | C | A | 1 | 0.7 | 495 |
| rs10993994_4 | rs6481329 | 10-51199752 | A | G | 1 | 0.7 | 248 |
| rs10993994_4 | rs7910704 | 10-51199811 | T | C | 1 | 0.28 | 284 |
| rs10993994_4 | rs4554834 | 10-51200152 | C | A | 1 | 0.7 | 217 |
| rs10993994_4 | rs10826125 | 10-51200511 | A | G | 1 | 0.7 | 35 |
| rs10993994_4 | rs10826127 | 10-51200763 | A | G | 1 | 0.73 | 36 |
| rs10993994_4 | rs4486572 | 10-51201811 | G | A | 1 | 0.7 | 209 |
| rs10993994_4 | rs4581397 | 10-51202373 | G | A | 0.95 | 0.69 | 221 |
| rs10993994_4 | rs4630240 | 10-51202534 | A | G | 1 | 0.32 | 223 |
| rs10993994_4 | rs7920517 | 10-51202627 | A | G | 1 | 0.7 | 286 |
| rs10993994_4 | rs4630241 | 10-51202757 | A | G | 1 | 0.7 | 224 |
| rs10993994_4 | rs9787697 | 10-51203382 | T | C | 1 | 0.7 | 293 |
| rs10993994_4 | rs10763534 | 10-51204926 | T | C | 1 | 0.7 | 19 |
| rs10993994_4 | rs10763536 | 10-51205807 | A | G | 1 | 0.7 | 20 |
| rs10993994_4 | s.51205998 | 10-51205998 | T | C | 1 | 0.7 | 496 |
| rs10993994_4 | rs10763546 | 10-51206405 | G | C | 1 | 0.68 | 21 |
| rs10993994_4 | s.51206890 | 10-51206890 | A | C | 0.74 | 0.54 | 497 |
| rs10993994_4 | rs4131357 | 10-51207298 | A | C | 1 | 0.7 | 196 |
| rs10993994_4 | s.51207437 | 10-51207437 | T | C | 1 | 0.7 | 498 |
| rs10993994_4 | s.51207481 | 10-51207481 | A | G | 1 | 0.7 | 499 |
| rs10993994_4 | s.51208175 | 10-51208175 | C | A | 0.85 | 0.58 | 500 |
| rs10993994_4 | rs11006207 | 10-51208182 | C | T | 1 | 0.7 | 64 |
| rs10993994_4 | rs10763576 | 10-51208819 | T | A | 1 | 0.7 | 22 |
| rs10993994_4 | s.51208921 | 10-51208921 | T | G | 1 | 0.68 | 501 |
| rs10993994_4 | rs11593361 | 10-51209162 | G | A | 1 | 0.68 | 90 |
| rs10993994_4 | rs10763588 | 10-51209768 | T | G | 1 | 0.7 | 23 |
| rs10993994_4 | rs11006274 | 10-51210297 | C | T | 1 | 0.7 | 65 |
| rs10993994_4 | s.51210619 | 10-51210619 | C | A | 0.74 | 0.54 | 502 |
| rs10993994_4 | s.51210866 | 10-51210866 | A | G | 1 | 0.7 | 503 |
| rs10993994_4 | rs4630243 | 10-51210873 | C | T | 1 | 0.7 | 225 |
| rs10993994_4 | rs4512771 | 10-51210912 | A | C | 1 | 0.7 | 211 |
| rs10993994_4 | rs4306255 | 10-51212450 | G | A | 1 | 0.7 | 204 |
| rs10993994_4 | s.51213076 | 10-51213076 | G | T | 1 | 0.68 | 504 |
| rs10993994_4 | rs4631830 | 10-51213350 | T | C | 0.95 | 0.69 | 226 |
| rs10993994_4 | rs7075009 | 10-51214149 | G | T | 1 | 0.7 | 260 |
| rs10993994_4 | rs7098889 | 10-51214481 | T | C | 1 | 0.7 | 270 |
| rs10993994_4 | rs4304716 | 10-51214593 | G | A | 0.85 | 0.58 | 203 |
| rs10993994_4 | s.51214689 | 10-51214689 | G | A | 1 | 0.29 | 505 |
| rs10993994_4 | s.51214690 | 10-51214690 | C | T | 1 | 0.68 | 506 |
| rs10993994_4 | rs7477953 | 10-51214698 | A | G | 1 | 0.7 | 279 |
| rs10993994_4 | s.51215034 | 10-51215034 | A | G | 0.95 | 0.66 | 507 |
| rs10993994_4 | s.51216121 | 10-51216121 | G | A | 0.86 | 0.21 | 508 |
| rs10993994_4 | s.51216342 | 10-51216342 | G | A | 1 | 0.81 | 509 |
| rs10993994_4 | rs7075697 | 10-51217377 | G | C | 0.95 | 0.66 | 261 |
| rs10993994_4 | s.51219226 | 10-51219226 | G | C | 0.9 | 0.65 | 510 |
| rs10993994_4 | s.51219227 | 10-51219227 | G | T | 1 | 0.63 | 511 |
| rs10993994_4 | s.51219230 | 10-51219230 | G | C | 1 | 0.37 | 512 |
| rs10993994_4 | s.51219320 | 10-51219320 | C | T | 1 | 0.63 | 513 |
| rs10993994_4 | s.51221179 | 10-51221179 | T | C | 1 | 0.42 | 514 |
| rs11067228_1 | s.113576401 | 12-113576401 | T | A | 1 | 0.41 | 296 |
| rs11067228_1 | s.113582477 | 12-113582477 | A | G | 1 | 1 | 297 |
| rs11067228_1 | s.113584188 | 12-113584188 | A | G | 1 | 0.84 | 298 |
| rs11067228_1 | s.113584539 | 12-113584539 | A | G | 1 | 0.3 | 299 |
| rs11067228_1 | s.113585097 | 12-113585097 | C | T | 1 | 0.81 | 300 |
| rs11067228_1 | rs12819162 | 12-113586774 | G | A | 0.82 | 0.23 | 110 |
| rs11067228_1 | rs11609105 | 12-113586865 | C | A | 0.91 | 0.32 | 93 |
| rs11067228_1 | rs514849 | 12-113588873 | A | G | 0.89 | 0.24 | 237 |
| rs11067228_1 | rs513061 | 12-113589060 | C | T | 0.89 | 0.24 | 236 |
| rs11067228_1 | s.113590733 | 12-113590733 | C | A | 0.96 | 0.74 | 301 |
| rs11067228_1 | rs1061657 | 12-113592519 | C | T | 0.91 | 0.32 | 13 |
| rs11067228_1 | rs8853 | 12-113593290 | T | C | 0.96 | 0.72 | 290 |
| rs11067228_1 | rs3741698 | 12-113593606 | G | C | 0.91 | 0.32 | 186 |
| rs11067228_1 | s.113594635 | 12-113594635 | T | G | 0.92 | 0.68 | 302 |
| rs11067228_1 | rs567223 | 12-113594954 | G | T | 0.89 | 0.76 | 242 |
| rs11067228_1 | rs551510 | 12-113598419 | C | T | 0.84 | 0.61 | 240 |
| rs11067228_1 | rs59336 | 12-113600735 | T | A | 0.8 | 0.58 | 245 |
| rs11067228_1 | s.113601412 | 12-113601412 | T | G | 0.83 | 0.27 | 303 |
| rs11067228_1 | rs515746 | 12-113603380 | G | A | 0.8 | 0.58 | 238 |
| rs11067228_1 | rs545076 | 12-113604286 | G | A | 0.8 | 0.58 | 239 |
| rs11067228_1 | s.113614584 | 12-113614584 | G | C | 0.62 | 0.22 | 304 |
| rs4430796_1 | rs3744763 | 17-33164998 | G | A | 0.67 | 0.37 | 187 |
| rs4430796_1 | rs7405776 | 17-33167135 | A | G | 1 | 0.78 | 278 |
| rs4430796_1 | rs2005705 | 17-33170413 | A | G | 1 | 1 | 128 |
| rs4430796_1 | s.33170591 | 17-33170591 | C | T | 1 | 0.63 | 454 |
| rs4430796_1 | rs11263761 | 17-33171888 | G | A | 1 | 0.44 | 87 |
| rs4430796_1 | rs4239217 | 17-33173100 | G | A | 1 | 0.67 | 201 |
| rs4430796_1 | rs11651755 | 17-33173953 | C | T | 1 | 1 | 95 |
| rs4430796_1 | rs10908278 | 17-33174065 | T | A | 1 | 1 | 57 |
| rs4430796_1 | s.33174083 | 17-33174083 | C | T | 1 | 0.44 | 455 |
| rs4430796_1 | rs11657964 | 17-33174880 | A | G | 1 | 0.78 | 96 |
| rs4430796_1 | rs7501939 | 17-33175269 | T | C | 1 | 0.75 | 280 |
| rs4430796_1 | rs8064454 | 17-33175699 | A | C | 1 | 1 | 289 |
| rs4430796_1 | s.33175746 | 17-33175746 | G | T | 1 | 0.75 | 456 |
| rs4430796_1 | s.33176039 | 17-33176039 | G | A | 1 | 0.75 | 457 |
| rs4430796_1 | rs7405696 | 17-33176148 | G | C | 1 | 0.63 | 277 |
| rs4430796_1 | rs11651052 | 17-33176494 | A | G | 1 | 1 | 94 |
| rs4430796_1 | rs11263763 | 17-33177678 | G | A | 1 | 0.97 | 88 |
| rs4430796_1 | rs11658063 | 17-33177985 | C | G | 1 | 0.78 | 97 |
| rs4430796_1 | rs9913260 | 17-33180010 | A | G | 1 | 0.48 | 294 |
| rs4430796_1 | rs3760511 | 17-33180426 | T | G | 1 | 0.33 | 188 |
| rs4430796_1 | s.33182344 | 17-33182344 | T | C | 1 | 0.33 | 458 |
| rs17632542_4 | s.55554247 | 19-55554247 | G | A | 1 | 0.24 | 515 |
| rs17632542_4 | s.55566277 | 19-55566277 | C | T | 1 | 0.24 | 516 |
| rs17632542_4 | s.55582344 | 19-55582344 | G | C | 1 | 0.24 | 517 |
| rs17632542_4 | rs2546552 | 19-55588229 | T | G | 1 | 0.24 | 136 |
| rs17632542_4 | s.55596785 | 19-55596785 | G | T | 1 | 0.24 | 518 |
| rs17632542_4 | s.55597645 | 19-55597645 | T | A | 1 | 0.24 | 519 |
| rs17632542_4 | s.55598078 | 19-55598078 | C | A | 1 | 0.24 | 520 |
| rs17632542_4 | s.55600121 | 19-55600121 | T | A | 1 | 0.24 | 521 |
| rs17632542_4 | s.55605246 | 19-55605246 | T | G | 1 | 0.24 | 522 |
| rs17632542_4 | s.55606024 | 19-55606024 | C | A | 1 | 0.24 | 523 |
| rs17632542_4 | s.55607242 | 19-55607242 | A | G | 1 | 0.24 | 524 |
| rs17632542_4 | s.55624341 | 19-55624341 | A | C | 1 | 0.24 | 525 |
| rs17632542_4 | s.55630396 | 19-55630396 | C | T | 1 | 0.24 | 526 |
| rs17632542_4 | s.55630578 | 19-55630578 | C | T | 0.72 | 0.25 | 527 |
| rs17632542_4 | s.55630679 | 19-55630679 | C | T | 0.72 | 0.25 | 528 |
| rs17632542_4 | s.55630791 | 19-55630791 | C | T | 0.72 | 0.25 | 529 |
| rs17632542_4 | s.55631170 | 19-55631170 | A | C | 1 | 0.24 | 530 |
| rs17632542_4 | s.55632347 | 19-55632347 | T | A | 1 | 0.24 | 531 |
| rs17632542_4 | s.55632363 | 19-55632363 | T | A | 1 | 0.24 | 532 |
| rs17632542_4 | s.55636052 | 19-55636052 | C | T | 1 | 0.24 | 533 |
| rs17632542_4 | s.55637350 | 19-55637350 | A | C | 1 | 0.24 | 534 |
| rs17632542_4 | s.55640040 | 19-55640040 | C | T | 1 | 0.24 | 535 |
| rs17632542_4 | s.55646568 | 19-55646568 | G | A | 1 | 0.24 | 536 |
| rs17632542_4 | s.55649132 | 19-55649132 | C | T | 1 | 0.24 | 537 |
| rs17632542_4 | s.55650629 | 19-55650629 | C | A | 1 | 0.24 | 538 |
| rs17632542_4 | s.55650844 | 19-55650844 | C | G | 1 | 0.24 | 539 |
| rs17632542_4 | s.55652397 | 19-55652397 | A | G | 1 | 0.24 | 540 |
| rs17632542_4 | s.55653401 | 19-55653401 | C | T | 1 | 0.24 | 541 |
| rs17632542_4 | s.55653991 | 19-55653991 | T | A | 1 | 0.24 | 542 |
| rs17632542_4 | s.55654907 | 19-55654907 | C | A | 1 | 0.24 | 543 |
| rs17632542_4 | s.55657973 | 19-55657973 | A | G | 1 | 0.24 | 544 |
| rs17632542_4 | s.55659043 | 19-55659043 | G | A | 1 | 0.24 | 545 |
| rs17632542_4 | s.55660011 | 19-55660011 | A | G | 1 | 0.24 | 546 |
| rs17632542_4 | s.55660013 | 19-55660013 | C | T | 1 | 0.24 | 547 |
| rs17632542_4 | s.55660139 | 19-55660139 | A | T | 1 | 0.24 | 548 |
| rs17632542_4 | s.55660143 | 19-55660143 | A | T | 1 | 0.24 | 549 |
| rs17632542_4 | s.55661660 | 19-55661660 | T | C | 1 | 0.24 | 550 |
| rs17632542_4 | s.55661718 | 19-55661718 | A | T | 1 | 0.24 | 551 |
| rs17632542_4 | rs6509476 | 19-55661773 | C | A | 1 | 0.24 | 249 |
| rs17632542_4 | s.55664020 | 19-55664020 | C | G | 1 | 0.24 | 552 |
| rs17632542_4 | s.55664897 | 19-55664897 | A | T | 1 | 0.24 | 553 |
| rs17632542_4 | s.55665723 | 19-55665723 | C | G | 0.72 | 0.25 | 554 |
| rs17632542_4 | s.55665726 | 19-55665726 | C | G | 1 | 0.24 | 555 |
| rs17632542_4 | s.55672641 | 19-55672641 | T | C | 1 | 0.24 | 556 |
| rs17632542_4 | s.55673254 | 19-55673254 | A | G | 0.72 | 0.25 | 557 |
| rs17632542_4 | s.55674252 | 19-55674252 | C | G | 1 | 0.24 | 558 |
| rs17632542_4 | s.55674254 | 19-55674254 | T | A | 1 | 0.24 | 559 |
| rs17632542_4 | s.55674727 | 19-55674727 | A | T | 1 | 0.24 | 560 |
| rs17632542_4 | s.55676073 | 19-55676073 | T | A | 1 | 0.24 | 561 |
| rs17632542_4 | s.55683393 | 19-55683393 | A | G | 1 | 0.24 | 562 |
| rs17632542_4 | s.55687122 | 19-55687122 | T | A | 1 | 0.24 | 563 |
| rs17632542_4 | s.55695317 | 19-55695317 | T | A | 1 | 0.24 | 564 |
| rs17632542_4 | s.55697027 | 19-55697027 | A | C | 1 | 0.24 | 565 |
| rs17632542_4 | s.55701748 | 19-55701748 | A | C | 0.72 | 0.25 | 566 |
| rs17632542_4 | rs7257447 | 19-55702303 | A | T | 1 | 0.24 | 273 |
| rs17632542_4 | s.55702308 | 19-55702308 | T | A | 1 | 0.24 | 567 |
| rs17632542_4 | s.55703568 | 19-55703568 | A | T | 1 | 0.24 | 568 |
| rs17632542_4 | s.55706751 | 19-55706751 | A | T | 1 | 0.24 | 569 |
| rs17632542_4 | s.55708051 | 19-55708051 | A | T | 1 | 0.24 | 570 |
| rs17632542_4 | s.55709067 | 19-55709067 | T | A | 1 | 0.24 | 571 |
| rs17632542_4 | s.55709498 | 19-55709498 | G | T | 1 | 0.24 | 572 |
| rs17632542_4 | s.55709766 | 19-55709766 | A | T | 1 | 0.24 | 573 |
| rs17632542_4 | s.55710030 | 19-55710030 | G | C | 1 | 0.24 | 574 |
| rs17632542_4 | s.55710848 | 19-55710848 | A | T | 1 | 0.24 | 575 |
| rs17632542_4 | s.55710851 | 19-55710851 | T | A | 1 | 0.24 | 576 |
| rs17632542_4 | s.55711749 | 19-55711749 | G | A | 0.72 | 0.25 | 577 |
| rs17632542_4 | s.55712802 | 19-55712802 | C | G | 1 | 0.24 | 578 |
| rs17632542_4 | s.55713451 | 19-55713451 | G | T | 1 | 0.24 | 579 |
| rs17632542_4 | s.55713453 | 19-55713453 | T | G | 1 | 0.24 | 580 |
| rs17632542_4 | s.55713458 | 19-55713458 | A | C | 1 | 0.24 | 581 |
| rs17632542_4 | s.55713862 | 19-55713862 | A | T | 1 | 0.24 | 582 |
| rs17632542_4 | s.55716007 | 19-55716007 | T | G | 1 | 0.24 | 583 |
| rs17632542_4 | s.55718272 | 19-55718272 | T | A | 1 | 0.24 | 584 |
| rs17632542_4 | s.55723496 | 19-55723496 | T | C | 0.72 | 0.25 | 585 |
| rs17632542_4 | s.55724346 | 19-55724346 | C | T | 1 | 0.24 | 586 |
| rs17632542_4 | s.55726794 | 19-55726794 | T | G | 1 | 0.24 | 587 |
| rs17632542_4 | s.55729556 | 19-55729556 | C | A | 1 | 0.24 | 588 |
| rs17632542_4 | s.55729562 | 19-55729562 | T | G | 1 | 0.24 | 589 |
| rs17632542_4 | s.55729563 | 19-55729563 | C | A | 1 | 0.24 | 590 |
| rs17632542_4 | s.55731588 | 19-55731588 | A | G | 0.72 | 0.25 | 591 |
| rs17632542_4 | s.55733658 | 19-55733658 | T | G | 1 | 0.24 | 592 |
| rs17632542_4 | s.55741403 | 19-55741403 | G | C | 1 | 0.24 | 593 |
| rs17632542_4 | s.55743524 | 19-55743524 | G | T | 1 | 0.24 | 594 |
| rs17632542_4 | s.55745833 | 19-55745833 | T | A | 1 | 0.24 | 595 |
| rs17632542_4 | s.55746123 | 19-55746123 | C | T | 1 | 0.24 | 596 |
| rs17632542_4 | s.55747079 | 19-55747079 | G | T | 1 | 0.24 | 597 |
| rs17632542_4 | s.55748269 | 19-55748269 | A | T | 1 | 0.24 | 598 |
| rs17632542_4 | s.55748274 | 19-55748274 | C | T | 1 | 0.24 | 599 |
| rs17632542_4 | s.55748844 | 19-55748844 | G | T | 1 | 0.24 | 600 |
| rs17632542_4 | s.55749193 | 19-55749193 | A | G | 1 | 0.24 | 601 |
| rs17632542_4 | s.55752178 | 19-55752178 | C | T | 1 | 0.24 | 602 |
| rs17632542_4 | s.55752271 | 19-55752271 | T | A | 1 | 0.24 | 603 |
| rs17632542_4 | s.55770158 | 19-55770158 | G | A | 1 | 0.24 | 604 |
| rs17632542_4 | rs7247686 | 19-55770361 | C | T | 1 | 0.24 | 272 |
| rs17632542_4 | s.55771401 | 19-55771401 | C | T | 1 | 0.24 | 605 |
| rs17632542_4 | s.55772266 | 19-55772266 | G | C | 1 | 0.24 | 606 |
| rs17632542_4 | s.55775314 | 19-55775314 | A | C | 1 | 0.24 | 607 |
| rs17632542_4 | s.55778756 | 19-55778756 | C | G | 1 | 0.24 | 608 |
| rs17632542_4 | s.55788661 | 19-55788661 | A | G | 1 | 0.24 | 609 |
| rs17632542_4 | s.55790622 | 19-55790622 | C | T | 1 | 0.24 | 610 |
| rs17632542_4 | s.55791942 | 19-55791942 | G | A | 1 | 0.24 | 611 |
| rs17632542_4 | rs10413426 | 19-55797671 | A | G | 1 | 0.24 | 11 |
| rs17632542_4 | s.55798366 | 19-55798366 | T | G | 1 | 0.24 | 612 |
| rs17632542_4 | s.55818900 | 19-55818900 | C | G | 1 | 0.24 | 613 |
| rs17632542_4 | s.55822129 | 19-55822129 | T | C | 1 | 0.24 | 614 |
| rs17632542_4 | s.55825528 | 19-55825528 | A | G | 1 | 0.24 | 615 |
| rs17632542_4 | s.55825624 | 19-55825624 | G | T | 1 | 0.24 | 616 |
| rs17632542_4 | s.55833489 | 19-55833489 | C | T | 1 | 0.24 | 617 |
| rs17632542_4 | s.55833938 | 19-55833938 | A | G | 1 | 0.24 | 618 |
| rs17632542_4 | s.55848124 | 19-55848124 | C | G | 1 | 0.24 | 619 |
| rs17632542_4 | s.55848125 | 19-55848125 | C | G | 1 | 0.24 | 620 |
| rs17632542_4 | s.55849044 | 19-55849044 | G | A | 1 | 0.24 | 621 |
| rs17632542_4 | s.55857289 | 19-55857289 | G | T | 1 | 0.24 | 622 |
| rs17632542_4 | s.55857585 | 19-55857585 | T | A | 1 | 0.24 | 623 |
| rs17632542_4 | s.55861107 | 19-55861107 | T | G | 1 | 0.24 | 624 |
| rs17632542_4 | s.55861111 | 19-55861111 | C | A | 1 | 0.24 | 625 |
| rs17632542_4 | s.55861196 | 19-55861196 | C | T | 1 | 0.24 | 626 |
| rs17632542_4 | s.55862851 | 19-55862851 | C | T | 1 | 0.24 | 627 |
| rs17632542_4 | s.55865439 | 19-55865439 | C | T | 1 | 0.24 | 628 |
| rs17632542_4 | s.55867208 | 19-55867208 | T | A | 1 | 0.24 | 629 |
| rs17632542_4 | s.55867650 | 19-55867650 | T | G | 1 | 0.24 | 630 |
| rs17632542_4 | s.55868902 | 19-55868902 | A | G | 1 | 0.24 | 631 |
| rs17632542_4 | s.55870429 | 19-55870429 | G | C | 1 | 0.24 | 632 |
| rs17632542_4 | rs73598616 | 19-55873660 | T | G | 1 | 0.24 | 276 |
| rs17632542_4 | s.55874339 | 19-55874339 | A | T | 1 | 0.24 | 633 |
| rs17632542_4 | s.55875249 | 19-55875249 | G | C | 1 | 0.24 | 634 |
| rs17632542_4 | s.55875725 | 19-55875725 | A | C | 1 | 0.24 | 635 |
| rs17632542_4 | s.55881262 | 19-55881262 | T | A | 1 | 0.24 | 636 |
| rs17632542_4 | s.55882788 | 19-55882788 | G | T | 1 | 0.24 | 637 |
| rs17632542_4 | s.55883542 | 19-55883542 | T | C | 1 | 0.24 | 638 |
| rs17632542_4 | s.55886467 | 19-55886467 | G | T | 1 | 0.24 | 639 |
| rs17632542_4 | s.55887498 | 19-55887498 | A | T | 1 | 0.24 | 640 |
| rs17632542_4 | s.55889175 | 19-55889175 | A | G | 1 | 0.24 | 641 |
| rs17632542_4 | s.55892113 | 19-55892113 | G | A | 1 | 0.24 | 642 |
| rs17632542_4 | s.55892618 | 19-55892618 | A | T | 1 | 0.24 | 643 |
| rs17632542_4 | s.55892866 | 19-55892866 | A | T | 1 | 0.24 | 644 |
| rs17632542_4 | s.55893305 | 19-55893305 | C | G | 1 | 0.24 | 645 |
| rs17632542_4 | s.55896443 | 19-55896443 | A | G | 1 | 0.24 | 646 |
| rs17632542_4 | s.55896826 | 19-55896826 | T | A | 1 | 0.24 | 647 |
| rs17632542_4 | s.55898241 | 19-55898241 | G | T | 1 | 0.24 | 648 |
| rs17632542_4 | s.55898245 | 19-55898245 | T | A | 1 | 0.24 | 649 |
| rs17632542_4 | s.55899120 | 19-55899120 | C | T | 1 | 0.24 | 650 |
| rs17632542_4 | s.55900597 | 19-55900597 | A | G | 1 | 0.24 | 651 |
| rs17632542_4 | s.55900764 | 19-55900764 | C | A | 1 | 0.24 | 652 |
| rs17632542_4 | s.55912567 | 19-55912567 | C | T | 1 | 0.24 | 653 |
| rs17632542_4 | s.55914840 | 19-55914840 | G | A | 1 | 0.24 | 654 |
| rs17632542_4 | s.55915776 | 19-55915776 | T | G | 1 | 0.24 | 655 |
| rs17632542_4 | s.55936192 | 19-55936192 | G | T | 1 | 0.24 | 656 |
| rs17632542_4 | s.55940336 | 19-55940336 | T | C | 1 | 0.24 | 657 |
| rs17632542_4 | s.55946316 | 19-55946316 | A | G | 1 | 0.24 | 658 |
| rs17632542_4 | s.55949971 | 19-55949971 | G | C | 1 | 0.24 | 659 |
| rs17632542_4 | s.55955333 | 19-55955333 | A | G | 1 | 0.24 | 660 |
| rs17632542_4 | s.55962188 | 19-55962188 | A | T | 1 | 0.24 | 661 |
| rs17632542_4 | s.55963864 | 19-55963864 | A | G | 1 | 0.24 | 662 |
| rs17632542_4 | s.55969754 | 19-55969754 | A | T | 1 | 0.24 | 663 |
| rs17632542_4 | s.55979135 | 19-55979135 | A | T | 1 | 0.24 | 664 |
| rs17632542_4 | rs67367861 | 19-55987833 | T | C | 1 | 0.24 | 252 |
| rs17632542_4 | s.55989580 | 19-55989580 | T | A | 1 | 0.24 | 665 |
| rs17632542_4 | s.56004001 | 19-56004001 | G | A | 1 | 0.24 | 666 |
| rs17632542_4 | s.56006528 | 19-56006528 | C | G | 1 | 0.24 | 667 |
| rs17632542_4 | s.56012046 | 19-56012046 | T | G | 1 | 0.24 | 668 |
| rs17632542_4 | s.56013739 | 19-56013739 | A | G | 1 | 0.24 | 669 |
| rs17632542_4 | rs2411330 | 19-56015173 | C | G | 1 | 0.24 | 134 |
| rs17632542_4 | rs3212825 | 19-56017315 | C | G | 1 | 0.24 | 176 |
| rs17632542_4 | s.56018053 | 19-56018053 | T | G | 1 | 0.24 | 670 |
| rs17632542_4 | s.56019106 | 19-56019106 | A | C | 1 | 0.24 | 671 |
| rs17632542_4 | rs7246740 | 19-56025486 | T | A | 1 | 0.24 | 271 |
| rs17632542_4 | s.56025860 | 19-56025860 | A | G | 1 | 0.24 | 672 |
| rs17632542_4 | s.56026713 | 19-56026713 | C | T | 1 | 0.24 | 673 |
| rs17632542_4 | rs55786312 | 19-56026861 | A | T | 1 | 0.21 | 241 |
| rs17632542_4 | s.56026881 | 19-56026881 | G | A | 1 | 0.24 | 674 |
| rs17632542_4 | s.56026882 | 19-56026882 | G | A | 1 | 0.24 | 675 |
| rs17632542_4 | s.56027319 | 19-56027319 | G | A | 1 | 0.24 | 676 |
| rs17632542_4 | s.56029265 | 19-56029265 | A | C | 1 | 0.24 | 677 |
| rs17632542_4 | s.56029362 | 19-56029362 | T | G | 1 | 0.24 | 678 |
| rs17632542_4 | s.56032778 | 19-56032778 | C | G | 1 | 0.24 | 679 |
| rs17632542_4 | s.56032963 | 19-56032963 | G | T | 1 | 0.24 | 680 |
| rs17632542_4 | s.56032964 | 19-56032964 | T | G | 1 | 0.24 | 681 |
| rs17632542_4 | s.56033138 | 19-56033138 | A | G | 0.82 | 0.49 | 682 |
| rs17632542_4 | s.56033138 | 19-56033138 | A | G | 1 | 0.43 | 682 |
| rs17632542_4 | s.56033664 | 19-56033664 | A | T | 1 | 0.21 | 683 |
| rs17632542_4 | s.56033664 | 19-56033664 | A | T | 1 | 0.36 | 683 |
| rs17632542_4 | s.56036363 | 19-56036363 | T | G | 1 | 0.24 | 684 |
| rs17632542_4 | s.56037076 | 19-56037076 | C | T | 1 | 0.36 | 685 |
| rs17632542_4 | s.56037076 | 19-56037076 | C | T | 1 | 0.61 | 685 |
| rs2735839_3 | rs2659051 | 19-56037380 | C | G | 0.61 | 0.27 | 145 |
| rs17632542_4 | s.56038334 | 19-56038334 | G | A | 1 | 0.28 | 686 |
| rs17632542_4 | s.56038334 | 19-56038334 | G | A | 1 | 0.48 | 686 |
| rs17632542_4 | s.56039736 | 19-56039736 | G | C | 1 | 0.24 | 687 |
| rs2735839_3 | rs266849 | 19-56040902 | G | A | 0.71 | 0.34 | 148 |
| rs17632542_4 | s.56042100 | 19-56042100 | G | C | 1 | 0.24 | 688 |
| rs17632542_4 | s.56042603 | 19-56042603 | G | A | 1 | 0.43 | 689 |
| rs17632542_4 | s.56042603 | 19-56042603 | G | A | 1 | 0.74 | 689 |
| rs17632542_4 | rs2659124 | 19-56046409 | A | T | 0.71 | 0.32 | 147 |
| rs17632542_4 | rs2659124 | 19-56046409 | A | T | 0.81 | 0.6 | 147 |
| rs17632542_4 | s.56046798 | 19-56046798 | T | C | 1 | 0.24 | 690 |
| rs17632542_4 | rs266878 | 19-56050926 | G | C | 0.7 | 0.26 | 149 |
| rs17632542_4 | rs266878 | 19-56050926 | G | C | 0.73 | 0.49 | 149 |
| rs17632542_4 | rs174776 | 19-56051664 | T | C | 0.7 | 0.26 | 113 |
| rs17632542_4 | rs174776 | 19-56051664 | T | C | 0.73 | 0.49 | 113 |
| rs17632542_4 | s.56052630 | 19-56052630 | C | T | 0.67 | 0.24 | 691 |
| rs17632542_4 | s.56052630 | 19-56052630 | C | T | 1 | 0.32 | 691 |
| rs17632542_4 | s.56052652 | 19-56052652 | T | C | 1 | 0.59 | 692 |
| rs17632542_4 | s.56052652 | 19-56052652 | T | C | 1 | 1 | 692 |
| rs2735839_3 | rs17632542 | 19-56053569 | C | T | 1 | 0.59 | 114 |
| rs17632542_4 | s.56053983 | 19-56053983 | G | C | 1 | 0.24 | 693 |
| rs17632542_4 | s.56054527 | 19-56054527 | G | T | 1 | 0.67 | 694 |
| rs17632542_4 | s.56054527 | 19-56054527 | G | T | 1 | 0.88 | 694 |
| rs2735839_3 | rs2659122 | 19-56054838 | C | T | 1 | 0.33 | 146 |
| rs17632542_4 | rs1058205 | 19-56055210 | C | T | 1 | 0.43 | 12 |
| rs17632542_4 | rs1058205 | 19-56055210 | C | T | 1 | 0.73 | 12 |
| rs17632542_4 | rs2569735 | 19-56056081 | A | G | 1 | 0.54 | 137 |
| rs17632542_4 | rs2569735 | 19-56056081 | A | G | 1 | 0.92 | 137 |
| rs17632542_4 | rs2735839 | 19-56056435 | A | G | 1 | 0.59 | 7 |
| rs17632542_4 | rs62113216 | 19-56056615 | A | T | 1 | 0.43 | 247 |
| rs17632542_4 | rs62113216 | 19-56056615 | A | T | 1 | 0.74 | 247 |
| rs17632542_4 | s.56058308 | 19-56058308 | A | G | 1 | 0.24 | 695 |
| rs17632542_4 | s.56058606 | 19-56058606 | T | A | 1 | 0.24 | 696 |
| rs17632542_4 | s.56058688 | 19-56058688 | A | T | 1 | 0.24 | 697 |
| rs17632542_4 | s.56058866 | 19-56058866 | C | T | 1 | 0.24 | 698 |
| rs17632542_4 | s.56060000 | 19-56060000 | C | A | 1 | 0.24 | 699 |
| rs17632542_4 | s.56061277 | 19-56061277 | C | G | 1 | 0.24 | 700 |
| rs17632542_4 | s.56062250 | 19-56062250 | A | C | 0.52 | 0.23 | 701 |
| rs17632542_4 | s.56066550 | 19-56066550 | A | T | 1 | 0.24 | 702 |
| rs17632542_4 | s.56066560 | 19-56066560 | G | C | 1 | 0.24 | 703 |
| rs17632542_4 | s.56066619 | 19-56066619 | T | G | 1 | 0.24 | 704 |
| rs17632542_4 | s.56067024 | 19-56067024 | T | C | 0.53 | 0.21 | 705 |
| rs17632542_4 | s.56067024 | 19-56067024 | T | C | 0.72 | 0.4 | 705 |
| rs17632542_4 | rs73592873 | 19-56074766 | A | G | 1 | 0.24 | 275 |
| rs17632542_4 | s.56076121 | 19-56076121 | C | G | 1 | 0.24 | 706 |
| rs17632542_4 | s.56076122 | 19-56076122 | C | G | 1 | 0.24 | 707 |
| rs17632542_4 | s.56078845 | 19-56078845 | C | G | 1 | 0.24 | 708 |
| rs17632542_4 | s.56085550 | 19-56085550 | C | G | 1 | 0.24 | 709 |
| rs17632542_4 | s.56093594 | 19-56093594 | T | G | 0.78 | 0.37 | 710 |
| rs17632542_4 | s.56472259 | 19-56472259 | A | C | 1 | 0.24 | 711 |
| rs2736098_4 | s.1030492 | 5-1030492 | A | G | 1 | 0.5 | 295 |
| rs2736098_4 | s.1233724 | 5-1233724 | G | C | 0.49 | 0.24 | 386 |
| rs2736098_4 | s.1251946 | 5-1251946 | G | C | 0.49 | 0.24 | 387 |
| rs2736098_4 | s.1257345 | 5-1257345 | G | A | 1 | 0.5 | 388 |
| rs2736098_4 | s.1258032 | 5-1258032 | A | G | 0.49 | 0.24 | 389 |
| rs401681_2 | rs9418 | 5-1278121 | C | T | 0.52 | 0.21 | 291 |
| rs401681_2 | s.1282167 | 5-1282167 | C | T | 0.68 | 0.22 | 390 |
| rs401681_2 | s.1285240 | 5-1285240 | C | T | 0.51 | 0.24 | 391 |
| rs401681_2 | s.1285775 | 5-1285775 | T | A | 0.53 | 0.23 | 392 |
| rs401681_2 | s.1287049 | 5-1287049 | G | A | 0.68 | 0.22 | 393 |
| rs2736098_4 | s.1292191 | 5-1292191 | T | C | 1 | 0.5 | 394 |
| rs2736098_4 | s.1334730 | 5-1334730 | C | A | 1 | 0.27 | 395 |
| rs401681_2 | s.1349759 | 5-1349759 | C | T | 0.63 | 0.22 | 396 |
| rs401681_2 | s.1350079 | 5-1350079 | C | A | 1 | 0.22 | 397 |
| rs401681_2 | rs2736108 | 5-1350488 | C | T | 0.63 | 0.22 | 158 |
| rs401681_2 | s.1350854 | 5-1350854 | C | T | 0.63 | 0.22 | 398 |
| rs401681_2 | rs2735948 | 5-1352213 | A | G | 0.78 | 0.51 | 156 |
| rs401681_2 | rs2735846 | 5-1352379 | C | G | 0.64 | 0.24 | 153 |
| rs401681_2 | s.1352392 | 5-1352392 | A | G | 1 | 0.28 | 399 |
| rs401681_2 | s.1353401 | 5-1353401 | T | C | 0.59 | 0.34 | 400 |
| rs401681_2 | rs2735946 | 5-1353429 | T | G | 0.94 | 0.51 | 155 |
| rs401681_2 | rs2736102 | 5-1355144 | T | C | 0.94 | 0.51 | 157 |
| rs401681_2 | rs2853666 | 5-1355914 | G | A | 0.95 | 0.68 | 166 |
| rs401681_2 | rs2735945 | 5-1356901 | T | C | 0.94 | 0.51 | 154 |
| rs401681_2 | s.1359165 | 5-1359165 | T | C | 0.96 | 0.71 | 401 |
| rs401681_2 | rs4530805 | 5-1359331 | T | C | 0.96 | 0.71 | 215 |
| rs401681_2 | s.1359765 | 5-1359765 | C | G | 0.96 | 0.8 | 402 |
| rs401681_2 | rs61574973 | 5-1362168 | T | C | 0.96 | 0.71 | 246 |
| rs401681_2 | s.1362904 | 5-1362904 | G | A | 0.96 | 0.9 | 403 |
| rs401681_2 | s.1363152 | 5-1363152 | G | A | 0.96 | 0.77 | 404 |
| rs401681_2 | rs12332579 | 5-1364198 | C | T | 0.89 | 0.23 | 101 |
| rs401681_2 | rs6866783 | 5-1365020 | T | C | 0.96 | 0.71 | 253 |
| rs401681_2 | s.1365329 | 5-1365329 | T | C | 1 | 0.24 | 405 |
| rs401681_2 | rs13356727 | 5-1365457 | G | A | 0.96 | 0.77 | 112 |
| rs401681_2 | rs13355267 | 5-1365935 | T | C | 0.96 | 0.77 | 111 |
| rs401681_2 | s.1366701 | 5-1366701 | A | G | 0.96 | 0.74 | 406 |
| rs401681_2 | rs10078017 | 5-1367009 | C | T | 0.96 | 0.77 | 10 |
| rs401681_2 | rs4975615 | 5-1368343 | G | A | 0.96 | 0.71 | 234 |
| rs401681_2 | rs4975616 | 5-1368660 | G | A | 0.96 | 0.8 | 235 |
| rs401681_2 | rs6554759 | 5-1370102 | G | A | 1 | 0.29 | 250 |
| rs401681_2 | rs3816659 | 5-1370820 | A | G | 1 | 0.93 | 190 |
| rs401681_2 | rs1801075 | 5-1370949 | C | T | 1 | 0.31 | 115 |
| rs401681_2 | rs451360 | 5-1372680 | A | C | 1 | 0.28 | 212 |
| rs401681_2 | rs421629 | 5-1373136 | A | G | 1 | 1 | 199 |
| rs401681_2 | rs380286 | 5-1373247 | A | G | 1 | 1 | 189 |
| rs401681_2 | rs402710 | 5-1373722 | T | C | 1 | 0.29 | 195 |
| rs401681_2 | rs10073340 | 5-1374873 | T | C | 1 | 0.29 | 9 |
| rs401681_2 | rs414965 | 5-1377121 | A | G | 1 | 0.93 | 197 |
| rs401681_2 | rs421284 | 5-1378590 | C | T | 1 | 0.93 | 198 |
| rs401681_2 | rs466502 | 5-1378767 | G | A | 1 | 0.97 | 228 |
| rs401681_2 | rs465498 | 5-1378803 | G | A | 1 | 0.97 | 227 |
| rs401681_2 | rs452932 | 5-1383253 | C | T | 1 | 1 | 214 |
| rs401681_2 | rs452384 | 5-1383840 | C | T | 1 | 1 | 213 |
| rs401681_2 | rs370348 | 5-1384219 | G | A | 1 | 1 | 185 |
| rs401681_2 | s.1386077 | 5-1386077 | G | A | 1 | 0.93 | 407 |
| rs401681_2 | s.1386169 | 5-1386169 | A | G | 1 | 0.65 | 408 |
| rs401681_2 | s.1386204 | 5-1386204 | A | G | 1 | 0.51 | 409 |
| rs401681_2 | s.1386674 | 5-1386674 | C | G | 1 | 0.35 | 410 |
| rs401681_2 | rs457130 | 5-1389178 | T | A | 1 | 0.87 | 219 |
| rs401681_2 | rs467095 | 5-1389221 | C | T | 1 | 0.9 | 229 |
| rs401681_2 | s.1389243 | 5-1389243 | G | A | 1 | 0.97 | 411 |
| rs401681_2 | rs462608 | 5-1389626 | A | T | 1 | 0.93 | 222 |
| rs401681_2 | rs456366 | 5-1390070 | C | T | 1 | 0.65 | 218 |
| rs401681_2 | s.1390106 | 5-1390106 | A | T | 1 | 0.97 | 412 |
| rs401681_2 | s.1390174 | 5-1390174 | C | T | 1 | 0.35 | 413 |
| rs401681_2 | rs31487 | 5-1394101 | C | G | 1 | 1 | 172 |
| rs401681_2 | s.1395154 | 5-1395154 | C | T | 1 | 0.47 | 414 |
| rs401681_2 | rs31489 | 5-1395714 | A | C | 1 | 0.93 | 173 |
| rs401681_2 | rs31490 | 5-1397458 | A | G | 1 | 1 | 174 |
| rs401681_2 | rs27996 | 5-1398474 | G | A | 1 | 0.93 | 159 |
| rs401681_2 | rs27071 | 5-1399081 | C | T | 1 | 0.47 | 152 |
| rs401681_2 | rs27070 | 5-1399303 | C | G | 1 | 0.9 | 151 |
| rs401681_2 | rs27068 | 5-1400239 | T | C | 0.93 | 0.43 | 150 |
| rs401681_2 | s.1401106 | 5-1401106 | C | T | 0.86 | 0.56 | 415 |
| rs401681_2 | rs37011 | 5-1401798 | T | A | 0.92 | 0.8 | 184 |
| rs401681_2 | s.1402130 | 5-1402130 | C | G | 1 | 0.45 | 416 |
| rs401681_2 | s.1402535 | 5-1402535 | G | A | 0.87 | 0.64 | 417 |
| rs401681_2 | rs37009 | 5-1403339 | T | C | 0.93 | 0.83 | 183 |
| rs401681_2 | rs40182 | 5-1403397 | A | G | 0.93 | 0.83 | 194 |
| rs401681_2 | rs37008 | 5-1404538 | A | G | 0.96 | 0.9 | 182 |
| rs401681_2 | rs37007 | 5-1405372 | C | G | 0.93 | 0.83 | 181 |
| rs401681_2 | s.1407027 | 5-1407027 | G | A | 1 | 0.32 | 418 |
| rs401681_2 | rs40181 | 5-1407462 | T | G | 0.92 | 0.8 | 193 |
| rs2736098_4 | s.1407682 | 5-1407682 | T | A | 1 | 0.5 | 419 |
| rs401681_2 | rs37006 | 5-1408058 | T | C | 0.93 | 0.83 | 180 |
| rs401681_2 | s.1408859 | 5-1408859 | T | C | 1 | 0.24 | 420 |
| rs401681_2 | rs37005 | 5-1409450 | T | C | 0.96 | 0.9 | 179 |
| rs401681_2 | s.1409771 | 5-1409771 | C | A | 0.93 | 0.83 | 421 |
| rs401681_2 | rs37002 | 5-1409944 | T | C | 0.93 | 0.83 | 178 |
| rs401681_2 | s.1411822 | 5-1411822 | T | C | 1 | 0.22 | 422 |
| rs401681_2 | s.1411901 | 5-1411901 | C | T | 0.83 | 0.27 | 423 |
| rs401681_2 | s.1412098 | 5-1412098 | T | C | 1 | 0.28 | 424 |
| rs401681_2 | rs31494 | 5-1414669 | T | G | 1 | 0.55 | 175 |
| rs401681_2 | s.1418662 | 5-1418662 | C | T | 1 | 0.28 | 425 |
| rs401681_2 | s.1419748 | 5-1419748 | A | G | 1 | 0.28 | 426 |
| rs2736098_4 | s.1426206 | 5-1426206 | A | T | 1 | 0.39 | 427 |
| rs2736098_4 | s.1426336 | 5-1426336 | C | T | 1 | 0.5 | 428 |
| rs2736098_4 | s.1428371 | 5-1428371 | C | A | 1 | 0.39 | 429 |
| rs2736098_4 | s.1428373 | 5-1428373 | C | A | 1 | 0.66 | 430 |
| rs2736098_4 | s.1472454 | 5-1472454 | C | T | 1 | 0.5 | 431 |
| rs2736098_4 | s.1518154 | 5-1518154 | A | C | 1 | 0.21 | 432 |
| rs2736098_4 | s.1557827 | 5-1557827 | C | A | 0.49 | 0.24 | 433 |
| rs2736098_4 | rs11743119 | 5-1583020 | G | C | 1 | 0.21 | 98 |
| rs2736098_4 | s.1583465 | 5-1583465 | T | A | 1 | 0.5 | 434 |
| rs2736098_4 | rs4551123 | 5-1589257 | A | G | 1 | 0.21 | 216 |
| rs2736098_4 | s.1589581 | 5-1589581 | C | G | 1 | 0.21 | 435 |
| rs2736098_4 | s.1591616 | 5-1591616 | G | C | 1 | 0.24 | 436 |
| rs2736098_4 | s.1607388 | 5-1607388 | C | T | 1 | 0.32 | 437 |
| rs2736098_4 | rs6893515 | 5-1615555 | C | T | 0.49 | 0.24 | 255 |
| rs2736098_4 | s.1618305 | 5-1618305 | G | C | 1 | 0.5 | 438 |
| rs2736098_4 | s.1621550 | 5-1621550 | T | C | 0.49 | 0.24 | 439 |
| rs2736098_4 | s.1621551 | 5-1621551 | G | A | 0.49 | 0.24 | 440 |
| rs2736098_4 | rs6892057 | 5-1630411 | C | G | 1 | 0.5 | 254 |
| rs2736098_4 | s.1638061 | 5-1638061 | T | C | 1 | 0.5 | 441 |
| rs2736098_4 | rs6898387 | 5-1638354 | T | C | 1 | 0.5 | 256 |
| rs2736098_4 | rs7724451 | 5-1649038 | A | G | 1 | 0.5 | 281 |
| rs2736098_4 | rs2937006 | 5-1662778 | G | A | 1 | 0.5 | 169 |
| rs2736098_4 | s.1663985 | 5-1663985 | G | T | 1 | 0.5 | 442 |
| rs2736098_4 | s.1667254 | 5-1667254 | G | A | 1 | 0.5 | 443 |
| rs2736098_4 | s.1668831 | 5-1668831 | C | T | 1 | 0.5 | 444 |
| rs2736098_4 | s.1673499 | 5-1673499 | G | A | 1 | 0.5 | 445 |
| rs2736098_4 | s.1737379 | 5-1737379 | A | G | 0.49 | 0.24 | 446 |
| rs2736098_4 | s.1756873 | 5-1756873 | C | A | 0.49 | 0.24 | 447 |
| rs2736098_4 | s.1782909 | 5-1782909 | A | G | 1 | 0.5 | 448 |
| rs2736098_4 | s.1788485 | 5-1788485 | G | C | 1 | 0.5 | 449 |
| rs2736098_4 | s.1799150 | 5-1799150 | G | A | 1 | 0.5 | 450 |
| rs2736098_4 | s.1800043 | 5-1800043 | G | T | 1 | 0.5 | 451 |
| rs2736098_4 | s.1804565 | 5-1804565 | G | A | 1 | 0.5 | 452 |
| rs2736098_4 | s.1812409 | 5-1812409 | A | G | 1 | 0.5 | 453 |
| rs2736098_4 | s.886453 | 5-886453 | A | G | 1 | 0.5 | 712 |
| rs2736098_4 | s.887600 | 5-887600 | T | C | 1 | 0.5 | 713 |
| rs10993994_4 | rs2012677 | 10-51174803 | T | A | 1 | 0.65 | 714 |
| rs4430796_1 | rs757210 | 17-33170628 | A | G | 0.96 | 0.61 | 715 |
| rs4430796_1 | rs7213769 | 17-33189279 | C | G | 0.73 | 0.27 | 716 |
| rs10788160_1 | rs11199892 | 10-123066171 | C | T | 0.77 | 0.29 | 717 |
| rs10788160_1 | rs11593067 | 10-122962348 | C | T | 0.76 | 0.20 | 718 |
| rs11067228_1 | rs12820376 | 12-113587344 | G | A | 0.91 | 0.24 | 719 |
| rs17632542_4 | rs273622 | 19-56486259 | G | A | 1 | 0.27 | 720 |
| rs401681_2 | rs2736098 | 5-1347086 | G | A | 0.94 | 0.39 | 721 |
| rs2736098_1 | rs2735845 | 5-1353584 | G | C | 0.71 | 0.26 | 722 |
| rs4430796_1 | rs1016990 | 17-33163028 | G | C | 0.56 | 0.21 | 723 |
| rs2736098_1 | rs31484 | 5-1390906 | T | A | 0.94 | 0.39 | 724 |
| rs401681_2 | rs31484 | 5-1390906 | T | A | 1 | 1.00 | 724 |

Shown are (1) anchor marker name and the allele correlating with increased PSA levels; (2) the surrogate marker; (3) chromosome and position of the surrogate marker in NCBI Build 36; (4) identity of the surrogate allele predicted to correlate with reduced PSA levels; (5) identity of the surrogate allele predicted to correlate with elevated PSA levels; (6) D′ values for the correlation between the anchor and the surrogate; and (7) r² values for the correlation between the anchor and the surrogate.

Suitable markers in linkage disequilibrium with any one of rs401681, rs2736098, rs10788160, rs10993994, rs11067228, rs4430796, rs2735839 and rs17632542 may for example be selected using the data provided in Table 1.

In one embodiment, suitable markers in linkage disequilibrium with rs401681 are selected from the group consisting of rs2736098, rs31484, rs4635969, rs9418, s.1282167, s.1285240, s.1285775, s.1287049, s.1349759, s.1350079, rs2736108, s.1350854, rs2735948, rs2735846, s.1352392, s.1353401, rs2735946, rs2736102, rs2853666, rs2735945, s.1359165, rs4530805, s.1359765, rs61574973, s.1362904, s.1363152, rs12332579, rs6866783, s.1365329, rs13356727, rs13355267, s.1366701, rs10078017, rs4975615, rs4975616, rs6554759, rs3816659, rs1801075, rs451360, rs421629, rs380286, rs402710, rs10073340, rs414965, rs421284, rs466502, rs465498, rs452932, rs452384, rs370348, s.1386077, s.1386169, s.1386204, s.1386674, rs457130, rs467095, s.1389243, rs462608, rs456366, s.1390106, s.1390174, rs31487, s.1395154, rs31489, rs31490, rs27996, rs27071, rs27070, rs27068, s.1401106, rs37011, s.1402130, s.1402535, rs37009, rs40182, rs37008, rs37007, s.1407027, rs40181, rs37006, s.1408859, rs37005, s.1409771, rs37002, s.1411822, s.1411901, s.1412098, rs31494, s.1418662, and s.1419748.

In one embodiment, suitable markers in linkage disequilibrium with rs2736098 are selected from the group consisting of rs2735845, rs31484, rs401681, s.1030492, s.1233724, s.1251946, s.1257345, s.1258032, s.1292191, s.1334730, s.1407682, s.1426206, s.1426336, s.1428371, s.1428373, s.1472454, s.1518154, s.1557827, rs11743119, s.1583465, rs4551123, s.1589581, s.1591616, s.1607388, rs6893515, s.1618305, s.1621550, s.1621551, rs6892057, s.1638061, rs6898387, rs7724451, rs2937006, s.1663985, s.1667254, s.1668831, s.1673499, s.1737379, s.1756873, s.1782909, s.1788485, s.1799150, s.1800043, s.1804565, s.1812409, s.886453, and s.887600.

In one embodiment, suitable markers in linkage disequilibrium with rs10788160 are selected from the group consisting of rs11199892, rs11593067, s.122837469, rs2130779, s.122876448, s.122901140, s.122901142, s.122905335, rs10788149, rs10749408, rs2172071, rs11592107, rs1907218, rs1907220, rs1994655, rs1907221, rs1907225, rs1907226, rs10749409, rs11199835, s.122991926, rs729014, s.122993518, s.122994309, s.122994946, rs1873450, rs2901290, s.122998594, s.122998678, s.122998978, rs2201026, rs4237529, s.122999386, rs1873451, rs1873452, rs4752520, rs10886880, rs10749412, s.123008216, rs3925042, rs1125527, rs1125528, rs4319451, rs10788154, rs7081844, rs7076500, s.123011774, s.123011879, rs11199862, s.123014171, rs12146156, s.123014499, s.123014519, rs12146366, s.123014684, rs7091083, rs7074985, rs7915008, s.123015342, s.123015365, rs10749413, rs11199866, s.123016003, rs7923130, rs7922901, rs10886882, rs10886883, rs11199867, s.123017698, s.123018111, rs4393247, s.123018188, rs4489674, rs11199868, s.123018670, s.123019408, s.123019759, rs11199869, s.123020245, s.123020365, rs10886885, rs10788159, rs10886886, rs11199871, rs11199872, rs12761612, rs4575197, rs11199874, rs10886887, s.123023625, s.123023836, rs4465316, rs4468286, rs10886890, rs10788162, s.123028135, rs12413648, s.123029102, rs10788163, s.123031617, s.123031811, rs10788164, rs11598592, rs10788165, rs9630106, rs10886893, s.123034821, rs11199879, rs11199881, rs12415826, rs10788166, rs10886894, rs10886895, rs10886896, rs10886897, rs10886898, rs10886899, rs10886900, rs10886901, rs10886902, rs10886903, rs12413088, rs10788167, s.123047182, rs7085073, rs7071101, rs12570783, rs11199884, rs7085506, rs10886905, rs10736302, s.123061811, s.123062031, rs11199886, s.123063327, s.123063715, rs10886907, s.123064252, s.123064345, s.123064780, s.123064783, s.123066424, s.123066700, rs3981043, rs11199896, rs11199897, rs11199898, s.123067963, rs11199900, rs11199901, s.123068178, s.123068222, s.123068236, s.123068424, s.123068619, s.123068743, s.123068926, s.123068997, s.123069012, s.123069326, s.123069570, s.123069989, s.123070105, s.123071090, s.123071347, rs4254007, s.123071495, s.123071914, s.123072804, rs7900630, s.123074016, rs1896416, s.123074531, s.123074928, s.123076274, s.123076472, rs2420925, s.123077398, s.123077455, rs12779205, rs11199912, rs4752534, s.123078389, rs1896420, rs1896419, s.123079199, s.123081990, s.123081993, s.123081998, and s.123201870.

In one embodiment, suitable markers in linkage disequilibrium with rs10993994 are selected from the group consisting of s.51157005, s.51159221, rs35716372, s.51159373, s.51159376, s.51159399, s.51159786, rs4935090, rs12781411, s.51162137, s.51162792, s.51162795, rs11004246, s.51165690, rs11004324, rs2843562, rs11004409, rs11004415, rs11004422, s.51168415, rs11004435, rs11599333, s.51170094, s.51170307, rs12763717, rs67289834, s.51172442, s.51172558, rs57858801, s.51172618, s.51172808, s.51173184, rs7071471, rs7090326, s.51173565, s.51173983, s.51174391, s.51174499, s.51174610, s.51174944, s.51175013, s.51175409, s.51176290, s.51176963, s.51180209, rs10825652, s.51180819, rs2843560, rs2125770, rs2611513, rs2611512, rs2611509, s.51186305, rs2926494, rs2611508, rs2611507, s.51188694, rs2611506, rs57263518, s.51189522, rs3101227, rs2843549, rs2843550, rs2249986, rs2843551, s.51192126, rs7077830, s.51193219, rs2843554, s.51194280, rs2611489, rs3123078, rs4935162, rs7081532, rs10826075, rs7896156, s.51199599, rs6481329, rs7910704, rs4554834, rs10826125, rs10826127, rs4486572, rs4581397, rs4630240, rs7920517, rs4630241, rs9787697, rs10763534, rs10763536, s.51205998, rs10763546, s.51206890, rs4131357, s.51207437, s.51207481, s.51208175, rs11006207, rs10763576, s.51208921, rs11593361, rs10763588, rs11006274, s.51210619, s.51210866, rs4630243, rs4512771, rs4306255, s.51213076, rs4631830, rs7075009, rs7098889, rs4304716, s.51214689, s.51214690, rs7477953, s.51215034, s.51216121, s.51216342, rs7075697, s.51219226, s.51219227, s.51219230, s.51219320, s.51221179, and rs2012677.

In one embodiment, suitable markers in linkage disequilibrium with rs11067228 are selected from the group consisting of rs12820376, s.113576401, s.113582477, s.113584188, s.113584539, s.113585097, rs12819162, rs11609105, rs514849, rs513061, s.113590733, rs1061657, rs8853, rs3741698, s.113594635, rs567223, rs551510, rs59336, s.113601412, rs515746, rs545076, and s.113614584.

In one embodiment, suitable markers in linkage disequilibrium with rs4430796 are selected from the group consisting of rs757210, rs7213769, rs1016990, rs17626423, rs3744763, rs7405776, rs2005705, s.33170591, rs11263761, rs4239217, rs11651755, rs10908278, s.33174083, rs11657964, rs7501939, rs8064454, s.33175746, s.33176039, rs7405696, rs11651052, rs11263763, rs11658063, rs9913260, rs3760511, and s.33182344.

In one embodiment, suitable markers in linkage disequilibrium with rs2735839 are selected from the group consisting of rs2659051, rs266849, rs17632542, and rs2659122. In one embodiment, suitable markers in linkage disequilibrium with rs17632542 are selected from the group consisting of rs273622, s.55554247, s.55566277, s.55582344, rs2546552, s.55596785, s.55597645, s.55598078, s.55600121, s.55605246, s.55606024, s.55607242, s.55624341, s.55630396, s.55630578, s.55630679, s.55630791, s.55631170, s.55632347, s.55632363, s.55636052, s.55637350, s.55640040, s.55646568, s.55649132, s.55650629, s.55650844, s.55652397, s.55653401, s.55653991, s.55654907, s.55657973, s.55659043, s.55660011, s.55660013, s.55660139, s.55660143, s.55661660, s.55661718, rs6509476, s.55664020, s.55664897, s.55665723, s.55665726, s.55672641, s.55673254, s.55674252, s.55674254, s.55674727, s.55676073, s.55683393, s.55687122, s.55695317, s.55697027, s.55701748, rs7257447, s.55702308, s.55703568, s.55706751, s.55708051, s.55709067, s.55709498, s.55709766, s.55710030, s.55710848, s.55710851, s.55711749, s.55712802, s.55713451, s.55713453, s.55713458, s.55713862, s.55716007, s.55718272, s.55723496, s.55724346, s.55726794, s.55729556, s.55729562, s.55729563, s.55731588, s.55733658, s.55741403, s.55743524, s.55745833, s.55746123, s.55747079, s.55748269, s.55748274, s.55748844, s.55749193, s.55752178, s.55752271, s.55770158, rs7247686, s.55771401, s.55772266, s.55775314, s.55778756, s.55788661, s.55790622, s.55791942, rs10413426, s.55798366, s.55818900, s.55822129, s.55825528, s.55825624, s.55833489, s.55833938, s.55848124, s.55848125, s.55849044, s.55857289, s.55857585, s.55861107, s.55861111, s.55861196, s.55862851, s.55865439, s.55867208, s.55867650, s.55868902, s.55870429, rs73598616, s.55874339, s.55875249, s.55875725, s.55881262, s.55882788, s.55883542, s.55886467, s.55887498, s.55889175, s.55892113, s.55892618, s.55892866, s.55893305, s.55896443, s.55896826, s.55898241, s.55898245, s.55899120, s.55900597, s.55900764, s.55912567, s.55914840, s.55915776, s.55936192, s.55940336, s.55946316, s.55949971, s.55955333, s.55962188, s.55963864, s.55969754, s.55979135, rs67367861, s.55989580, s.56004001, s.56006528, s.56012046, s.56013739, rs2411330, rs3212825, s.56018053, s.56019106, rs7246740, s.56025860, s.56026713, rs55786312, s.56026881, s.56026882, s.56027319, s.56029265, s.56029362, s.56032778, s.56032963, s.56032964, s.56033138, s.56033138, s.56033664, s.56033664, s.56036363, s.56037076, s.56037076, s.56038334, s.56038334, s.56039736, s.56042100, s.56042603, s.56042603, rs2659124, rs2659124, s.56046798, rs266878, rs266878, rs174776, rs174776, s.56052630, s.56052630, s.56052652, s.56052652, s.56053983, s.56054527, s.56054527, rs1058205, rs1058205, rs2569735, rs2569735, rs2735839, rs62113216, rs62113216, s.56058308, s.56058606, s.56058688, s.56058866, s.56060000, s.56061277, s.56062250, s.56066550, s.56066560, s.56066619, s.56067024, s.56067024, rs73592873, s.56076121, s.56076122, s.56078845, s.56085550, s.56093594, and s.56472259.

The skilled person will appreciate that using the LD data provided in Table 1, suitable surrogate markers may be selected based on suitable cutoff values for the LD measures r² and D′.

### Detecting Polymorphic Markers

Alleles for SNP markers as referred to herein refer to the bases A, C, G or T as they occur at the polymorphic site. The allele codes for SNPs used herein are as follows: 1=A, 2=C, 3=G, 4=T. Since human DNA is double-stranded, the person skilled in the art will realise that by assaying or reading the opposite DNA strand, the complementary allele can in each case be measured. Thus, for a polymorphic site (polymorphic marker) characterized by an A/G polymorphism, the methodology employed to detect the marker may be designed to specifically detect the presence of one or both of the two bases possible, i.e. A and G. Alternatively, by designing an assay that is designed to detect the complimentary strand on the DNA template, the presence of the complementary bases T and C can be measured. Quantitatively (for example, in terms of risk estimates), identical results would be obtained from measurement of either DNA strand (+ strand or − strand).

A haplotype refers to a single-stranded segment of DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles, each allele corresponding to a specific polymorphic marker along the segment. Haplotypes can comprise a combination of various polymorphic markers, e.g., SNPs and microsatellites, having particular alleles at the polymorphic sites. The haplotypes thus comprise a combination of alleles at various genetic markers.

It is possible to impute or predict genotypes for un-genotyped relatives of genotyped individuals. For every un-genotyped case, it is possible to calculate the probability of the genotypes of its relatives given its four possible phased genotypes. In practice it may be preferable to include only the genotypes of the case's parents, children, siblings, half-siblings (and the half-sibling's parents), grand-parents, grand-children (and the grand-children's parents) and spouses. It will be assumed that the individuals in the small sub-pedigrees created around each case are not related through any path not included in the pedigree. It is also assumed that alleles that are not transmitted to the case have the same frequency—the population allele frequency. Let us consider a SNP marker with the alleles A and G. The probability of the genotypes of the case's relatives can then be computed by:

$$Pr ( genotypes of relatives ; θ ) = {\sum }_{h ∈ \{ AA , AG , GA , GG \}} Pr ( h ; θ ) Pr ( genotypes of relatives \vert h ) ,$$

where θ denotes the A allele's frequency in the cases. Assuming the genotypes of each set of relatives are independent, this allows us to write down a likelihood function for θ:

$$L ( θ ) = {\prod }_{i} Pr ( genotypesof relativesof case i ; θ ) . \qquad (* )$$

This assumption of independence is usually not correct. Accounting for the dependence between individuals is a difficult and potentially prohibitively expensive computational task. The likelihood function in (*) may be thought of as a pseudolikelihood approximation of the full likelihood function for θ which properly accounts for all dependencies. In general, the genotyped cases and controls in a case-control association study are not independent and applying the case-control method to related cases and controls is an analogous approximation. The method of genomic control (Devlin, B. et al., Nat Genet. 36, 1129-30; author reply 1131 (2004)) has proven to be successful at adjusting case-control test statistics for relatedness. We therefore apply the method of genomic control to account for the dependence between the terms in our pseudolikelihood and produce a valid test statistic.

Fisher's information can be used to estimate the effective sample size of the part of the pseudolikelihood due to un-genotyped cases. Breaking the total Fisher information, I, into the part due to genotyped cases, I_{g}, and the part due to ungenotyped cases, Iᵤ, I=I_{g}+Iᵤ, and denoting the number of genotyped cases with N, the effective sample size due to the un-genotyped cases is estimated by

$$\frac{{I}_{u}}{{I}_{g}} N .$$

It is also possible to impute genotypes for markers with no genotype data. For example, using the IMPUTE (Marchini, J. et al. Nat Genet. 39:906-13 (2007)) software and the HapMap (NCBI Build 36 (db126b)) CEU data as reference (Frazer, K. A., et al. Nature 449:851-61 (2007)) it is possible to impute ungenotyped markers. This can be useful for extending genotype coverage, if the CEU dataset has been genotyped.

### Analyzing Multiple Markers

A genetic variant associated with a disease or a trait such as PSA quantity can be used alone to predict the risk of the disease for a given genotype. For a biallelic marker, such as a SNP, there are 3 possible genotypes: homozygote for the at risk variant, heterozygote, and non carrier of the at risk variant. Risk associated with variants at multiple loci can be used to estimate overall risk. For multiple SNP variants, there are k possible genotypes k=3ⁿ×2^{p}; where n is the number autosomal loci and p the number of gonosomal (sex chromosomal) loci. Overall risk assessment calculations for a plurality of risk variants usually assume that the relative risks of different genetic variants multiply, i.e. the overall risk (e.g., RR or OR) associated with a particular genotype combination is the product of the risk values for the genotype at each locus. If the risk presented is the relative risk for a person, or a specific genotype for a person, compared to a reference population with matched gender and ethnicity, then the combined risk is the product of the locus specific risk values and also corresponds to an overall risk estimate compared with the population. If the risk for a person is based on a comparison to non-carriers of the at risk allele, then the combined risk corresponds to an estimate that compares the person with a given combination of genotypes at all loci to a group of individuals who do not carry risk variants at any of those loci. The group of non-carriers of any at risk variant has the lowest estimated risk and has a combined risk, compared with itself (i.e., non-carriers) of 1.0, but has an overall risk, compare with the population, of less than 1.0. It should be noted that the group of non-carriers can potentially be very small, especially for large number of loci, and in that case, its relevance is correspondingly small.

The multiplicative model is a parsimonious model that usually fits the data of complex traits reasonably well. Deviations from multiplicity have been rarely described in the context of common variants for common diseases, and if reported are usually only suggestive since very large sample sizes are usually required to be able to demonstrate statistical interactions between loci.

By way of an example, let us consider a case of eight variants that have been associated with risk prostate cancer (Gudmundsson, J., et al., Nat Genet. 39:631-7 (2007), Gudmundsson, J., et al., Nat Genet. 39:977-83 (2007); Yeager, M., et al, Nat Genet. 39:645-49 (2007), Amundadottir, L., et al., Nat Genet. 38:652-8 (2006); Haiman, C. A., et al., Nat Genet. 39:638-44 (2007)). Seven of these loci are on autosomes, and the remaining locus is on chromosome X. The total number of theoretical genotypic combinations is then 3⁷×2¹=4374. Some of those genotypic classes are very rare, but are still possible, and should be considered for overall risk assessment.

It is likely that the multiplicative model applied in the case of multiple genetic variants will also be valid in conjugation with non-genetic risk variants assuming that the genetic variant does not clearly correlate with the “environmental” factor. In other words, genetic and non-genetic at-risk variants can be assessed under the multiplicative model to estimate combined risk, assuming that the non-genetic and genetic risk factors do not interact.

Using the same quantitative approach, the combined or overall risk associated with any plurality of variants associated with PSA quantity and prostate cancer risk, as described herein, may be assessed.

### Risk Assessment and Diagnostics

Within any given population, there is an absolute risk of developing a disease or trait, defined as the chance of a person developing the specific disease or trait over a specified time-period. For example, a woman's lifetime absolute risk of breast cancer is one in nine. That is to say, one woman in every nine will develop breast cancer at some point in their lives. Risk is typically measured by looking at very large numbers of people, rather than at a particular individual. Risk is often presented in terms of Absolute Risk (AR) and Relative Risk (RR). Relative Risk is used to compare risks associating with two variants or the risks of two different groups of people. For example, it can be used to compare a group of people with a certain genotype with another group having a different genotype. For a disease, a relative risk of 2 means that one group has twice the chance of developing a disease as the other group. The risk presented is usually the relative risk for a person, or a specific genotype of a person, compared to the population with matched gender and ethnicity. Risks of two individuals of the same gender and ethnicity could be compared in a simple manner. For example, if, compared to the population, the first individual has relative risk 1.5 and the second has relative risk 0.5, then the risk of the first individual compared to the second individual is 1.5/0.5=3.

### Risk Calculations

The creation of a model to calculate the overall genetic risk involves two steps: i) conversion of odds-ratios for a single genetic variant into relative risk and ii) combination of risk from multiple variants in different genetic loci into a single relative risk value.

Deriving Risk from Odds-Ratios

Most gene discovery studies for complex diseases that have been published to date in authoritative journals have employed a case-control design because of their retrospective setup. These studies sample and genotype a selected set of cases (people who have the specified disease condition) and control individuals. The interest is in genetic variants (alleles) which frequency in cases and controls differ significantly.

The results are typically reported in odds ratios, that is the ratio between the fraction (probability) with the risk variant (carriers) versus the non-risk variant (non-carriers) in the groups of affected versus the controls, i.e. expressed in terms of probabilities conditional on the affection status:

OR=(Pr(c|A)/Pr(nc|A))/(Pr(c|C)/Pr(nc|C))

Sometimes it is however the absolute risk for the disease that we are interested in, i.e. the fraction of those individuals carrying the risk variant who get the disease or in other words the probability of getting the disease. This number cannot be directly measured in case-control studies, in part, because the ratio of cases versus controls is typically not the same as that in the general population. However, under certain assumption, we can estimate the risk from the odds ratio.

It is well known that under the rare disease assumption, the relative risk of a disease can be approximated by the odds ratio. This assumption may however not hold for many common diseases. Still, it turns out that the risk of one genotype variant relative to another can be estimated from the odds ratio expressed above. The calculation is particularly simple under the assumption of random population controls where the controls are random samples from the same population as the cases, including affected people rather than being strictly unaffected individuals. To increase sample size and power, many of the large genome-wide association and replication studies use controls that were neither age-matched with the cases, nor were they carefully scrutinized to ensure that they did not have the disease at the time of the study. Hence, while not exactly, they often approximate a random sample from the general population. It is noted that this assumption is rarely expected to be satisfied exactly, but the risk estimates are usually robust to moderate deviations from this assumption.

Calculations show that for the dominant and the recessive models, where we have a risk variant carrier, “c”, and a non-carrier, “nc”, the odds ratio of individuals is the same as the risk ratio between these variants:

OR=Pr(A|c)/Pr(A|nc)=r

And likewise for the multiplicative model, where the risk is the product of the risk associated with the two allele copies, the allelic odds ratio equals the risk factor:

OR=Pr(A|aa)/Pr(A|ab)=Pr(A|ab)/Pr(A|bb)=r

Here “a” denotes the risk allele and “b” the non-risk allele. The factor “r” is therefore the relative risk between the allele types.

For many of the studies published in the last few years, reporting common variants associated with complex diseases, the multiplicative model has been found to summarize the effect adequately and most often provide a fit to the data superior to alternative models such as the dominant and recessive models.

### The Risk Relative to the Average Population Risk

It is most convenient to represent the risk of a genetic variant relative to the average population since it makes it easier to communicate the lifetime risk for developing the disease compared with the baseline population risk. For example, in the multiplicative model we can calculate the relative population risk for variant “aa” as:

RR(aa)=Pr(A|aa)/Pr(A)=(Pr(A|aa)/Pr(A|bb))/(Pr(A)/Pr(A|bb))=r²/(Pr(aa)r²+Pr(ab)r+Pr(bb))=r²/(p²r²+2pqr+q²)=r²/R

Here “p” and “q” are the allele frequencies of “a” and “b” respectively. Likewise, we get that RR(ab)=r/R and RR(bb)=1/R. The allele frequency estimates may be obtained from the publications that report the odds-ratios and from the HapMap database. Note that in the case where we do not know the genotypes of an individual, the relative genetic risk for that test or marker is simply equal to one.

Combining the Risk from Multiple Markers

When genotypes of many SNP variants are used to estimate the risk for an individual a multiplicative model for risk can generally be assumed. This means that the combined genetic risk relative to the population is calculated as the product of the corresponding estimates for individual markers, e.g. for two markers g1 and g2: RR(g1,g2)=RR(g1)RR(g2)

The underlying assumption is that the risk factors occur and behave independently, i.e. that the joint conditional probabilities can be represented as products:

Pr(A|g1,g2)=Pr(A|g1)Pr(A|g2)/Pr(A) and Pr(g1,g2)=Pr(g1)Pr(g2)

Obvious violations to this assumption are markers that are closely spaced on the genome, i.e. in linkage disequilibrium, such that the concurrence of two or more risk alleles is correlated. In such cases, we can use so called haplotype modeling where the odds-ratios are defined for all allele combinations of the correlated SNPs.

As is in most situations where a statistical model is utilized, the model applied is not expected to be exactly true since it is not based on an underlying bio-physical model. However, the multiplicative model has so far been found to fit the data adequately, i.e. no significant deviations are detected for many common diseases for which many risk variants have been discovered.

As an example, an individual who has the following genotypes at 4 hypothetical markers associated with a particular disease along with the risk relative to the population at each marker:

| Marker | Genotype | Calculated risk |
|---|---|---|
| M1 | CC | 1.03 |
| M2 | GG | 1.30 |
| M3 | AG | 0.88 |
| M4 | TT | 1.54 |

Combined, the overall risk relative to the population for this individual is: 1.03×1.30×0.88×1.54=1.81.

### Risk Assessment of Prostate Cancer

As described herein, certain polymorphic markers and haplotypes comprising such markers are found to be useful for risk assessment of prostate cancer. Certain markers have also been found to be useful for correcting PSA quantity to establish a corrected PSA quantity based on the genotype of individuals at particular polymorphic markers. Markers in linkage disequilibrium with any such marker are, by necessity, also useful in such applications. This fact is obvious to the skilled person, who thus knows that surrogate markers may be suitably selected to detect the effect of any particular anchor marker. The stronger the linkage disequilibrium to the anchor marker, the better the surrogate, and thus the more similar the results obtained by detecting the surrogate will be to that of the anchor marker. Markers with values of r² equal to 1 are perfect surrogates anchor marker, i.e. genotypes for the surrogate marker perfectly predicts genotypes for the anchor marker. Markers with smaller values of r² than 1 can also be useful surrogates, although they are expected to give rise to observed effects that are smaller than for the anchor marker. Alternatively, such surrogate markers may represent variants with effects (e.g., OR, RR for prostate cancer, or effect on PSA levels) as high as or possibly even higher than that of the anchor marker. In this scenario, the anchor variant identified may not be the functional variant itself, but is in this instance in linkage disequilibrium with the true functional variant. The functional variant may be a SNP, but may also for example be a tandem repeat, such as a minisatellite or a microsatellite, a transposable element (e.g., an Alu element), or a structural alteration, such as a deletion, insertion or inversion (sometimes also called copy number variations, or CNVs). The present invention encompasses the assessment of such surrogate markers for the markers as disclosed herein. Such markers are annotated, mapped and listed in public databases, as well known to the skilled person, or can alternatively be readily identified by sequencing a genomic region or a part of the region identified by the markers of the present invention in a group of individuals, and identify polymorphisms in the resulting group of sequences. As a consequence, the person skilled in the art can readily and without undue experimentation identify and genotype surrogate markers in linkage disequilibrium with the markers described herein.

Detection of nucleic acid sequence as described herein can in certain embodiments be practiced by assessing a sample comprising genomic DNA from an individual for the presence of certain variants described herein to be associated with PSA levels and risk of prostate cancer. Such assessment typically includes steps that detect the presence or absence of at least one allele of at least one polymorphic marker, using methods well known to the skilled person and further described herein, and based on the outcome of such assessment, determine whether the individual from whom the sample is derived is at increased or decreased risk (i.e., increased or decreased susceptibility) of prostate, or determine a corrected PSA value based on the outcome. Obtaining nucleic acid sequence data can comprise nucleic acid sequence at a single nucleotide position, which is sufficient to identify alleles at SNPs. The nucleic acid sequence data can also comprise sequence at any other number of nucleotide positions, in particular for genetic markers that comprise multiple nucleotide positions, and can be anywhere from two to hundreds of thousands, possibly even millions, of nucleotides (in particular, in the case of copy number variations (CNVs)).

In certain embodiments, the invention can be practiced utilizing a dataset comprising information about the genotype status of at least one polymorphic marker. In other words, a dataset containing information about particular polymorphic markers, for example in the form of genotype counts at a certain polymorphic marker, or a plurality of markers (e.g., an indication of the presence or absence of certain at-risk alleles, or the presence or absence of certain alleles predictive of increased or decreased PSA quantity), or actual genotypes for one or more markers, can be queried for the presence or absence of certain alleles.

It should be apparent to the skilled person that the methods described herein for determining corrected PSA quantity and methods of assessing prostate cancer susceptibility may be performed using multiple markers. Thus, any one, or a combination of the markers described herein may be used. In certain embodiments, the use of additional polymorphic markers useful in the method is contemplated. Methods known in the art and described herein may be used to determine the overall effect of such multiple markers.

### Study Population

The Icelandic population is a Caucasian population of Northern European ancestry. A large number of studies reporting results of genetic linkage and association in the Icelandic population have been published in the last few years. Many of those studies show replication of variants, originally identified in the Icelandic population as being associating with a particular disease, in other populations (Sulem, P., et al. Nat Genet May 17, 2009 (Epub ahead of print); Rafnar, T., et al. Nat Genet. 41:221-7 (2009); Gretarsdottir, S., et al. Ann Neurol 64:402-9 (2008); Stacey, S, N., et al. Nat Genet. 40:1313-18 (2008); Gudbjartsson, D. F., et al. Nat Genet. 40:886-91 (2008); Styrkarsdottir, U., et al. N Engl J Med 358:2355-65 (2008); Thorgeirsson, T., et al. Nature 452:638-42 (2008); Gudmundsson, J., et al. Nat. Genet. 40:281-3 (2008); Stacey, S. N., et al., Nat. Genet. 39:865-69 (2007); Helgadottir, A., et al., Science 316:1491-93 (2007); Steinthorsdottir, V., et al., Nat. Genet. 39:770-75 (2007); Gudmundsson, J., et al., Nat. Genet. 39:631-37 (2007); Frayling, T M, Nature Reviews Genet. 8:657-662 (2007); Amundadottir, L. T., et al., Nat Genet. 38:652-58 (2006); Grant, S. F., et al., Nat. Genet. 38:320-23 (2006)). Thus, genetic findings in the Icelandic population have in general been replicated in other populations, including populations from Africa and Asia.

By way of example, prostate cancer risk variants on Chromosome 8q24 (rs1447295 and rs16901979), Chromosome 17q12 (rs4430796), Chromosome 17q24.3 (rs1859962), Chromosome 2p15 (rs2710646), Chromosome 11q13 (rs10896450) and Chromosome Xp11.22 (rs5945572), all of which had originally been identified in samples from the Icelandic population have been confirmed as risk variants of prostate cancer in many other populations.

It is thus believed that the markers described herein to be associated with PSA quantity and prostate cancer risk will show similar association in other human populations. Particular embodiments comprising individual human populations are therefore also contemplated and within the scope of the invention. Such embodiments relate to human individuals that are from one or more human population including, but not limited to, Caucasian populations, European populations, American populations, Eurasian populations, Asian populations, Central/South Asian populations, East Asian populations, Middle Eastern populations, African populations, Hispanic populations, and Oceanian populations.

In certain embodiments, the invention relates to markers and/or haplotypes identified in specific populations, as described in the above. The person skilled in the art will appreciate that linkage disequilibrium (LD) may vary across human populations. This is due to different population history of different human populations as well as differential selective pressures that may have led to differences in LD in specific genomic regions. It is also well known to the person skilled in the art that certain markers, e.g. SNP markers, have different population frequency in different populations, or are polymorphic in one population but not in another. The person skilled in the art will however apply available methods and methods described herein to practice the present invention in any given human population. For example, selecting markers in LD with an anchor marker may in certain embodiments be done using Caucasian samples. In general, however, markers in LD with an anchor markers may be suitably selected using LD determined in a particular population that is intended for study. For example, for applying the present invention in the Chinese population, it may be suitable to select markers in LD with a particular anchor marker (e.g., any of the markers shown herein to be predictive of PSA quantity in humans) based on LD measures determined in samples from the Chinese population. Such selection of markers is well known to the skilled person, and can be done using data from the public domain, for example data from the HapMap project (available at hapmap.org), utilizing methods known in the art.

As a consequence, certain embodiments of the invention pertain to markers that are in linkage disequilibrium with a marker selected from the group consisting of rs401681, rs2736098, rs10788160, rs11067228, rs10993994, rs4430796, rs2735839 and rs17632542, wherein linkage disequilibrium is determined in samples from the same human population as the individual being studied. In certain embodiments, the individual is Caucasian and the population is a Caucasian population. The population may also suitably be a European population, for example in cases where the individual is European or of European origin. Certain other embodiments relate to populations with a European origin.

### Nucleic Acids and Polypeptides

The nucleic acids and polypeptides described herein can be used in methods and kits of the present invention. An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells or heterologous organisms, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Such isolated nucleotide sequences are useful, for example, in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis or other hybridization techniques.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a marker or haplotype described herein). Such nucleic acid molecules can be detected and/or isolated by allele- or sequence-specific hybridization (e.g., under high stringency conditions). Stringency conditions and methods for nucleic acid hybridizations are well known to the skilled person (see, e.g., Current Protocols in Molecular Biology, Ausubel, F. et al, John Wiley & Sons, (1998), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by well-known methods, for example, using a mathematical algorithm. A non-limiting example of such a mathematical algorithm is described in Karlin, S. and Altschul, S., Proc. Natl. Acad. Sci. USA, 90:5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, the default parameters of the respective programs (e.g., NBLAST) can be used. See the website on the World Wide Web at ncbi.nlm.nih.gov. In one embodiment, parameters for sequence comparison can be set at score=100, wordlength=12, or can be varied (e.g., W=5 or W=20). Another example of an algorithm is BLAT (Kent, W. J. Genome Res. 12:656-64 (2002)). Other examples include the algorithm of Myers and Miller, CABIOS (1989), ADVANCE and ADAM as described in Torellis, A. and Robotti, C., Comput. Appl. Biosci. 10:3-5 (1994); and FASTA described in Pearson, W. and Lipman, D., Proc. Natl. Acad. Sci. USA, 85:2444-48 (1988). In another embodiment, the percent identity between two amino acid sequences can be accomplished using the GAP program in the GCG software package (Accelrys, Cambridge, UK).

The present invention also provides isolated nucleic acid molecules that contain a fragment or portion that hybridizes under highly stringent conditions to a nucleic acid that comprises, or consists of, the nucleotide sequence of any one of the KLK3 gene, the HNF1B gene, the FGFR2 gene, the TBX3 gene, the MSMB gene and the TERT gene, or a nucleotide sequence comprising, or consisting of, the complement of the nucleotide sequence of any one of the KLK3 gene, the HNF1B gene, the FGFR2 gene, the TBX3 gene, the MSMB gene and the TERT gene. In certain embodiments, the nucleotide sequence comprises at least one polymorphic allele contained in the markers described herein. The nucleic acid fragments of the invention are at least about 15, at least about 18, 20, 23 or 25 nucleotides, and can be 30, 40, 50, 100, 200, 500, 1000, 10,000 or more nucleotides in length. In a specific embodiment, the nucleic acid fragments are 15-500 nucleotides in length.

The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule. In one embodiment, the probe or primer comprises at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

The nucleic acid molecules of the invention, such as those described above, can be identified and isolated using standard molecular biology techniques well known to the skilled person. The amplified DNA can be labeled (e.g., radiolabeled, fluorescently labeled) and used as a probe for screening a cDNA library derived from human cells. The cDNA can be derived from mRNA and contained in a suitable vector. Corresponding clones can be isolated, DNA obtained following in vivo excision, and the cloned insert can be sequenced in either or both orientations by art-recognized methods to identify the correct reading frame encoding a polypeptide of the appropriate molecular weight. Using these or similar methods, the polypeptide and the DNA encoding the polypeptide can be isolated, sequenced and further characterized.

### Kits

Kits useful in the methods of the invention comprise components useful in any of the methods described herein, including for example, primers for nucleic acid amplification, hybridization probes, restriction enzymes (e.g., for RFLP analysis), allele-specific oligonucleotides, antibodies useful for detecting PSA, e.g. antibodies that bind to PSA epitopes, antibodies that bind to an altered PSA polypeptide (e.g., antibodies that bind to PSA epitopes that comprise a 1179T variation) or to a non-altered (native) polypeptide encoded, means for analyzing the nucleic acid sequence of a nucleic acid, etc. The kits can for include necessary buffers, nucleic acid primers for amplifying nucleic acids of the invention, and reagents for allele-specific detection of the fragments amplified using such primers and necessary enzymes (e.g., DNA polymerase). Additionally, kits can provide reagents for assays to be used in combination with the methods of the present invention, e.g., reagents for use with other diagnostic assays. For example, in certain embodiments, kits provide reagents for performing a PSA assay.

In one embodiment, the invention pertains to a kit for assaying a sample from a subject to detect a the presence or absence of certain alleles at certain polymorphic markers in a subject, wherein the kit comprises reagents necessary for selectively detecting at least one allele of at least one polymorphism as described herein in the genome of the individual. In a particular embodiment, the reagents comprise at least one contiguous oligonucleotide that hybridizes to a fragment of the genome of the individual comprising at least one polymorphism of the present invention. In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one polymorphism that is useful in the methods described herein. For example, in certain embodiments, the polymorphism is selected from the group consisting of rs401681, rs2736098, rs10788160, rs11067228, rs10993994, rs4430796, rs2735839 and rs17632542, and markers in linkage disequilibrium therewith. In one embodiment the fragment is at least 20 base pairs in size. Such oligonucleotides or nucleic acids (e.g., oligonucleotide primers) can be designed using portions of the nucleic acid sequence flanking polymorphisms (e.g., SNPs or microsatellites) that are associated with PSA levels, as described herein. In another embodiment, the kit comprises one or more labeled nucleic acids capable of allele-specific detection of one or more specific polymorphic markers, and reagents for detection of the label. Suitable labels include, e.g., a radioisotope, a fluorescent label, an enzyme label, an enzyme co-factor label, a magnetic label, a spin label, an epitope label.

In particular embodiments, the polymorphic marker or haplotype to be detected by the reagents of the kit comprises one or more markers, two or more markers, three or more markers, four or more markers, five or more markers, six or more markers, seven or more markers, eight or more markers, nine or more markers, or ten or more markers. In a further aspect of the present invention, a pack (kit) is provided, the pack comprising (i) reagents for determining PSA levels in humans, and (ii) reagents for determining sequence information about at least one polymorphic marker, wherein the at least one polymorphic marker is correlated with PSA quantity in humans. In certain embodiments, the reagents for determining sequence information comprise reagents for determining the presence or absence of at least one allele of at least one polymorphic marker.

In certain embodiments, the kit further comprises a set of instructions for using the reagents comprising the kit. In certain embodiments, the kit further comprises instructions for interpreting results obtained by using reagents in the kit. For example, the instructions in one embodiment comprise instructions for determining corrected PSA levels based on (a) uncorrected PSA levels obtained using reagents provided in the kit and (b) sequence information obtained using reagents provided in the kit. In another embodiment, the kit contains a data sheet providing information on corrected PSA values based on results on uncorrected PSA values and sequence information about at least one polymorphic marker obtained using the reagents provided in the kit.

### Antibodies

The invention also provides antibodies which bind to an epitope comprising either a variant amino acid sequence (e.g., comprising an amino acid substitution) encoded by a variant allele or the reference amino acid sequence encoded by the corresponding non-variant or wild-type allele. The term “antibody” as used herein refers to immunoglobulin molecules and immunologically active portions of immunoglobulin molecules, i.e., molecules that contain antigen-binding sites that specifically bind an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4: 72 (1983)), the EBV-hybridoma technique (Cole et al., Monoclonal Antibodies and Cancer Therapy, Alan R. Liss, 1985, Inc., pp. 77-96) or trioma techniques. The technology for producing hybridomas is well known (see generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9: 1370-1372 (1991); Hay et al., Hum. Antibod. Hybridomas 3:81-85 (1992); Huse et al., Science 246: 1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies may also be useful in pharmacogenomic analysis. In such embodiments, antibodies against variant proteins encoded by nucleic acids according to the invention, such as variant proteins that are encoded by nucleic acids that contain at least one polymorphic marker of the invention, can be used to identify individuals that require modified treatment modalities.

Antibodies can furthermore be useful for assessing expression of variant proteins in disease states, such as in active stages of a disease, or in an individual with a predisposition to a disease related to the function of the protein, in particular prostate cancer. In certain embodiments, antibodies are useful for assessing PSA quantity in humans. Antibodies specific for a variant protein of the present invention can be used to screen for the presence of the variant protein, for example to screen for a predisposition to prostate cancer as indicated by the presence of the variant protein. In one embodiment, the variant protein is a I179T variant of the KLK3 protein.

Antibodies can be used in other methods. Thus, antibodies are useful as diagnostic tools for evaluating proteins, such as variant proteins of the invention, in conjunction with analysis by electrophoretic mobility, isoelectric point, tryptic or other protease digest, or for use in other physical assays known to those skilled in the art. Antibodies may also be used in tissue typing. In one such embodiment, a specific variant protein has been correlated with expression in a specific tissue type, and antibodies specific for the variant protein can then be used to identify the specific tissue type.

Subcellular localization of proteins, including variant proteins, can also be determined using antibodies, and can be applied to assess aberrant subcellular localization of the protein in cells in various tissues. Such use can be applied in genetic testing, but also in monitoring a particular treatment modality. In the case where treatment is aimed at correcting the expression level or presence of the variant protein or aberrant tissue distribution or developmental expression of the variant protein, antibodies specific for the variant protein or fragments thereof can be used to monitor therapeutic efficacy.

Antibodies are further useful for inhibiting variant protein function, for example by blocking the binding of a variant protein to a binding molecule or partner. Such uses can also be applied in a therapeutic context in which treatment involves inhibiting a variant protein's function. An antibody can be for example be used to block or competitively inhibit binding, thereby modulating (i.e., agonizing or antagonizing) the activity of the protein. Antibodies can be prepared against specific protein fragments containing sites required for specific function or against an intact protein that is associated with a cell or cell membrane. For administration in vivo, an antibody may be linked with an additional therapeutic payload, such as radionuclide, an enzyme, an immunogenic epitope, or a cytotoxic agent, including bacterial toxins (diphtheria or plant toxins, such as ricin). The in vivo half-life of an antibody or a fragment thereof may be increased by pegylation through conjugation to polyethylene glycol.

The present invention further relates to kits for using antibodies in the methods described herein. This includes, but is not limited to, kits for detecting the quantity of protein in a sample, and kits for detecting the presence of a variant protein in a sample. One preferred embodiment comprises antibodies such as a labelled or labelable antibody and a compound or agent for detecting PSA in a biological sample and/or means for determining the quantity of PSA protein in the sample, as well as instructions for use of the kit.

### Antisense

The nucleic acids and/or variants described herein, or nucleic acids comprising their complementary sequence, may be used as antisense constructs to control gene expression in cells, tissues or organs. The methodology associated with antisense techniques is well known to the skilled artisan, and is for example described and reviewed in AntisenseDrug Technology: Principles, Strategies, and Applications, Crooke, ed., Marcel Dekker Inc., New York (2001). In general, antisense agents (antisense oligonucleotides) are comprised of single stranded oligonucleotides (RNA or DNA) that are capable of binding to a complimentary nucleotide segment. By binding the appropriate target sequence, an RNA-RNA, DNA-DNA or RNA-DNA duplex is formed. The antisense oligonucleotides are complementary to the sense or coding strand of a gene. It is also possible to form a triple helix, where the antisense oligonucleotide binds to duplex DNA.

Several classes of antisense oligonucleotide are known to those skilled in the art, including cleavers and blockers. The former bind to target RNA sites, activate intracellular nucleases (e.g., RnaseH or Rnase L), that cleave the target RNA. Blockers bind to target RNA, inhibit protein translation by steric hindrance of the ribosomes. Examples of blockers include nucleic acids, morpholino compounds, locked nucleic acids and methylphosphonates (Thompson, Drug Discovery Today, 7:912-917 (2002)). Antisense oligonucleotides are useful directly as therapeutic agents, and are also useful for determining and validating gene function, for example by gene knock-out or gene knock-down experiments. Antisense technology is further described in Layery et al., Curr. Opin. Drug Discov. Devel. 6:561-569 (2003), Stephens et al., Curr. Opin. Mol. Ther. 5:118-122 (2003), Kurreck, Eur. J. Biochem. 270:1628-44 (2003), Dias et al., Mol. Cancer. Ter. 1:347-55 (2002), Chen, Methods Mol. Med. 75:621-636 (2003), Wang et al., Curr. Cancer Drug Targets 1:177-96 (2001), and Bennett, Antisense Nucleic Acid Drug Dev. 12:215-24 (2002).

In certain embodiments, the antisense agent is an oligonucleotide that is capable of binding to a particular nucleotide segment. In certain embodiments, the nucleotide segment comprises a fragment of a gene selected from the group consisting of the KLK3 gene, the HNF1B gene, the FGFR2 gene, the TBX3 gene, the MSMB gene and the TERT gene. In certain other embodiments, the antisense nucleotide is capable of binding to a nucleotide segment of as set forth in SEQ ID NO:1-728. Antisense nucleotides can be from 5-500 nucleotides in length, including 5-200 nucleotides, 5-100 nucleotides, 10-50 nucleotides, and 10-30 nucleotides. In certain preferred embodiments, the antisense nucleotides are from 14-50 nucleotides in length, including 14-40 nucleotides and 14-30 nucleotides.

The variants described herein can also be used for the selection and design of antisense reagents that are specific for particular variants. Using information about the variants described herein, antisense oligonucleotides or other antisense molecules that specifically target mRNA molecules that contain one or more variants of the invention can be designed. In this manner, expression of mRNA molecules that contain one or more variant of the present invention (i.e. certain marker alleles and/or haplotypes) can be inhibited or blocked. In one embodiment, the antisense molecules are designed to specifically bind a particular allelic form (i.e., one or several variants (alleles and/or haplotypes)) of the target nucleic acid, thereby inhibiting translation of a product originating from this specific allele or haplotype, but which do not bind other or alternate variants at the specific polymorphic sites of the target nucleic acid molecule. As antisense molecules can be used to inactivate mRNA so as to inhibit gene expression, and thus protein expression, the molecules can be used for disease treatment. The methodology can involve cleavage by means of ribozymes containing nucleotide sequences complementary to one or more regions in the mRNA that attenuate the ability of the mRNA to be translated. Such mRNA regions include, for example, protein-coding regions, in particular protein-coding regions corresponding to catalytic activity, substrate and/or ligand binding sites, or other functional domains of a protein.

The phenomenon of RNA interference (RNAi) has been actively studied for the last decade, since its original discovery in C. elegans (Fire et al., Nature 391:806-11 (1998)), and in recent years its potential use in treatment of human disease has been actively pursued (reviewed in Kim & Rossi, Nature Rev. Genet. 8:173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a protein-RNA complex to specific sites on a target mRNA, leading to cleavage of the mRNA (Thompson, Drug Discovery Today, 7:912-917 (2002)). The siRNA molecules are typically about 20, 21, 22 or 23 nucleotides in length. Thus, one aspect of the invention relates to isolated nucleic acid molecules, and the use of those molecules for RNA interference, i.e. as small interfering RNA molecules (siRNA). In one embodiment, the isolated nucleic acid molecules are 18-26 nucleotides in length, preferably 19-25 nucleotides in length, more preferably 20-24 nucleotides in length, and more preferably 21, 22 or 23 nucleotides in length.

Another pathway for RNAi-mediated gene silencing originates in endogenously encoded primary microRNA (pri-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8:173-204 (2007)).

Clinical applications of RNAi include the incorporation of synthetic siRNA duplexes, which preferably are approximately 20-23 nucleotides in size, and preferably have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules (typically 25-30 nucleotides in length, preferably about 27 nucleotides), as well as small hairpin RNAs (shRNAs; typically about 29 nucleotides in length). The latter are naturally expressed, as described in Amarzguioui et al. (FEBS Lett. 579:5974-81 (2005)). Chemically synthetic siRNAs and shRNAs are substrates for in vivo processing, and in some cases provide more potent gene-silencing than shorter designs (Kim et al., Nature Biotechnol. 23:222-226 (2005); Siolas et al., Nature Biotechnol. 23:227-231 (2005)). In general siRNAs provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al., Nature Biotechnol. 23:559-565 (2006); Brummelkamp et al., Science 296: 550-553 (2002)).

Since RNAi molecules, including siRNA, miRNA and shRNA, act in a sequence-dependent manner, the variants presented herein can be used to design RNAi reagents that recognize specific nucleic acid molecules comprising specific alleles and/or haplotypes (e.g., the alleles and/or haplotypes of the present invention), while not recognizing nucleic acid molecules comprising other alleles or haplotypes. These RNAi reagents can thus recognize and destroy the target nucleic acid molecules. As with antisense reagents, RNAi reagents can be useful as therapeutic agents (i.e., for turning off disease-associated genes or disease-associated gene variants), but may also be useful for characterizing and validating gene function (e.g., by gene knock-out or gene knock-down experiments).

Delivery of RNAi may be performed by a range of methodologies known to those skilled in the art. Methods utilizing non-viral delivery include cholesterol, stable nucleic acid-lipid particle (SNALP), heavy-chain antibody fragment (Fab), aptamers and nanoparticles. Viral delivery methods include use of lentivirus, adenovirus and adeno-associated virus. The siRNA molecules are in some embodiments chemically modified to increase their stability. This can include modifications at the 2′ position of the ribose, including 2′-O-methylpurines and 2′-fluoropyrimidines, which provide resistance to Rnase activity. Other chemical modifications are possible and known to those skilled in the art.

### Prognostic Methods

In addition to the utilities described above, the polymorphic markers of the invention are useful in determining prognosis of human individuals. Accurate pretreatment staging is important for prostate cancer treatment. Serum PSA levels correlate with aggressiveness of disease. Thus, individuals with serum PSA levels less than 10 ng/mL are most likely to respond to local therapy. Further, the PSA velocity (change in levels per year) is an independent predictor of mortality following treatment.

Given the important contribution of genetic factors to PSA levels, it would be valuable to use corrected values of PSA quantity to assess prognosis. The invention therefore provides a method for determining the prognosis of an individual diagnosed with prostate cancer, the method comprising (i) detecting an uncorrected PSA quantity in a first biological sample from the human individual; (ii) obtaining sequence data about at least one polymorphic marker in the first biological sample or in a second biological sample from the human individual, wherein the at least one polymorphic marker is correlated with PSA quantity in humans; and (iii) determining a corrected PSA quantity in the human individual based on the sequence data about the at least one polymorphic marker; wherein the corrected PSA quantity is indicative of the prognosis of the individual. In one embodiment, a corrected PSA quantity of 10 ng/mL or greater is indicative of a worse prognosis.

In one embodiment, the method further comprises determining corrected PSA velocity by repeating steps (i)-(iii) using a first sample and/or a second sample taken at a different time than the first set of first and/or second sample, and calculating a corrected PSA velocity based on the corrected PSA quantity determined for samples obtained at different times.

In preferred embodiments, the at least one polymorphic marker is selected from the group consisting of rs401681, rs2736098, rs10788160, rs11067228, rs10993994, rs4430796, rs2735839 and rs17632542, and markers in linkage disequilibrium therewith.

### Methods of Assessing Recurrence Risk

PSA quantity is a useful tool for assessing recurrence risk in individuals who have undergone treatment for prostate cancer. Following treatment, PSA levels should decrease and remain at a low and steady level over time. A detection of an increased PSA levels in individuals who have undergone treatment is thus an indication of disease recurrence. Applying a correction of uncorrected PSA quantity, as described herein, is useful for this purpose. This is particularly important if a particular PSA threshold is used as a guidance that an individual is experiencing, or is at risk for, disease recurrence.

Therefore, the invention in a further aspect provides a method of assessing recurrence risk of prostate cancer in a human individual who has undergone treatment for prostate cancer, the method comprising (i) detecting an uncorrected PSA quantity in a first biological sample from the human individual; (ii) obtaining sequence data about at least one polymorphic marker in the first biological sample or in a second biological sample from the human individual, wherein the at least one polymorphic marker is correlated with PSA quantity in humans; and (iii) determining a corrected PSA quantity in the human individual based on the sequence data about the at least one polymorphic marker; wherein the corrected PSA quantity is indicative of recurrence risk of the individual. In certain embodiments, a corrected PSA quantity above a certain threshold is indicative of recurrence in the individual. In certain embodiments, a corrected PSA quantity of 0.5 or greater is indicative of recurrence in the individual. In one embodiment, a corrected PSA quantity of 1.0 or greater is indicative of recurrence in the individual. In another embodiment, a corrected PSA quantity of 2.0 or greater is indicative of recurrence in the individual. In another embodiment, a corrected PSA quantity of 3.0 or greater is indicative of recurrence in the individual. In another embodiment, a corrected PSA quantity of 4.0 or greater is indicative of recurrence in the individual.

In certain embodiments, the method further comprises determining corrected PSA velocity by repeating steps (i)-(iii) using a first sample and/or a second sample taken at a different time than the first set of first and/or second sample, and calculating a corrected PSA velocity based on the corrected PSA quantity determined for samples obtained at said different times.

The at least one polymorphic marker is suitably selected from the group consisting of rs401681, rs2736098, rs10788160, rs11067228, rs10993994, rs4430796, rs2735839 and rs17632542, and markers in linkage disequilibrium therewith.

### Computer-Implemented Aspects

As understood by those of ordinary skill in the art, the methods and information described herein may be implemented, in all or in part, as computer executable instructions on known computer readable media. For example, the methods described herein may be implemented in hardware. Alternatively, the method may be implemented in software stored in, for example, one or more memories or other computer readable medium and implemented on one or more processors. As is known, the processors may be associated with one or more controllers, calculation units and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the Internet, a wireless connection, etc., or via a transportable medium, such as a computer readable disk, flash drive, etc.

More generally, and as understood by those of ordinary skill in the art, the various steps described above may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any known computer readable medium such as on a magnetic disk, an optical disk, or other storage medium, in a RAM or ROM or flash memory of a computer, processor, hard disk drive, optical disk drive, tape drive, etc. Likewise, the software may be delivered to a user or a computing system via any known delivery method including, for example, on a computer readable disk or other transportable computer storage mechanism.

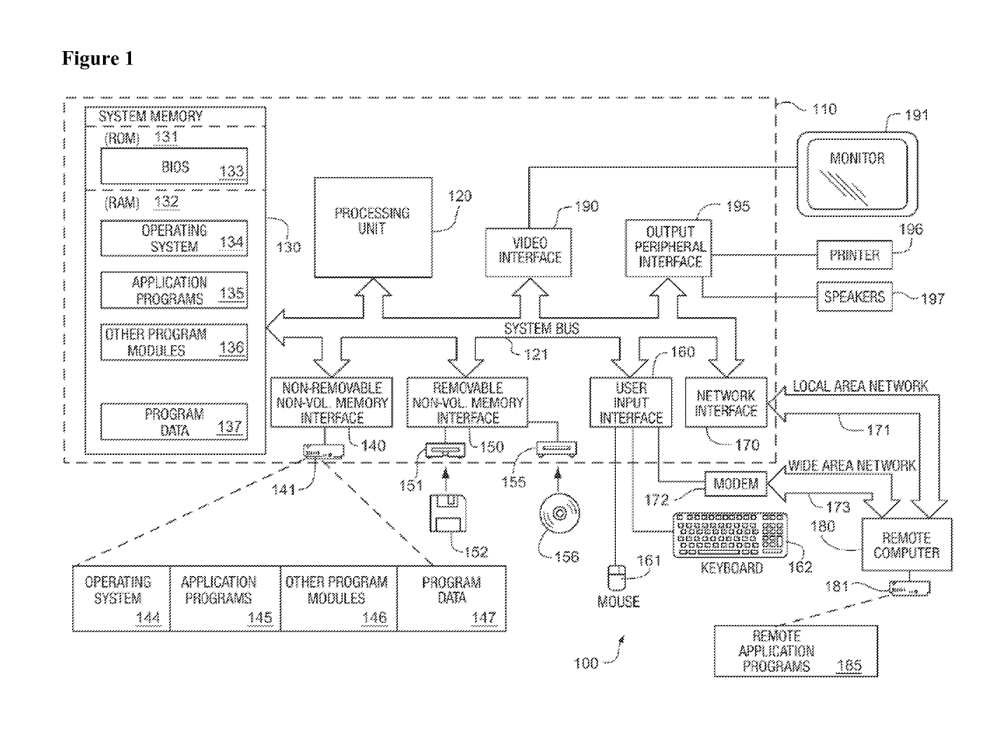

FIG. 1 illustrates an example of a suitable computing system environment 100 on which a system for the steps of the claimed method and apparatus may be implemented. The computing system environment 100 is only one example of a suitable computing environment and is not intended to suggest any limitation as to the scope of use or functionality of the method or apparatus of the claims. Neither should the computing environment 100 be interpreted as having any dependency or requirement relating to any one or combination of components illustrated in the exemplary operating environment 100.

The steps of the claimed method and system are operational with numerous other general purpose or special purpose computing system environments or configurations. Examples of well known computing systems, environments, and/or configurations that may be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The steps of the claimed method and system may be described in the general context of computer-executable instructions, such as program modules, being executed by a computer. Generally, program modules include routines, programs, objects, components, data structures, etc. that perform particular tasks or implement particular abstract data types. The methods and apparatus may also be practiced in distributed computing environments where tasks are performed by remote processing devices that are linked through a communications network. In both integrated and distributed computing environments, program modules may be located in both local and remote computer storage media including memory storage devices.

With reference to FIG. 1, an exemplary system for implementing the steps of the claimed method and system includes a general purpose computing device in the form of a computer 110. Components of computer 110 may include, but are not limited to, a processing unit 120, a system memory 130, and a system bus 121 that couples various system components including the system memory to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (USA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 110 typically includes a variety of computer readable media. Computer readable media can be any available media that can be accessed by computer 110 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 110. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 130 includes computer storage media in the form of volatile and/or nonvolatile memory such as read only memory (ROM) 131 and random access memory (RAM) 132. A basic input/output system 133 (BIOS), containing the basic routines that help to transfer information between elements within computer 110, such as during start-up, is typically stored in ROM 131. RAM 132 typically contains data and/or program modules that are immediately accessible to and/or presently being operated on by processing unit 120. By way of example, and not limitation, FIG. 1 illustrates operating system 134, application programs 135, other program modules 136, and program data 137.

The computer 110 may also include other removable/non-removable, volatile/nonvolatile computer storage media. By way of example only, FIG. 1 illustrates a hard disk drive 140 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives and their associated computer storage media discussed above and illustrated in FIG. 1, provide storage of computer readable instructions, data structures, program modules and other data for the computer 110. In FIG. 1, for example, hard disk drive 141 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a keyboard 162 and pointing device 161, commonly referred to as a mouse, trackball or touch pad. Other input devices (not shown) may include a microphone, joystick, game pad, satellite dish, scanner, or the like. These and other input devices are often connected to the processing unit 120 through a user input interface 160 that is coupled to the system bus, but may be connected by other interface and bus structures, such as a parallel port, game port or a universal serial bus (USB). A monitor 191 or other type of display device is also connected to the system bus 121 via an interface, such as a video interface 190. In addition to the monitor, computers may also include other peripheral output devices such as speakers 197 and printer 196, which may be connected through an output peripheral interface 190.

The computer 110 may operate in a networked environment using logical connections to one or more remote computers, such as a remote computer 180. The remote computer 180 may be a personal computer, a server, a router, a network PC, a peer device or other common network node, and typically includes many or all of the elements described above relative to the computer 110, although only a memory storage device 181 has been illustrated in FIG. 1. The logical connections depicted in FIG. 1 include a local area network (LAN) 171 and a wide area network (WAN) 173, but may also include other networks. Such networking environments are commonplace in offices, enterprise-wide computer networks, intranets and the Internet.

When used in a LAN networking environment, the computer 110 is connected to the LAN 171 through a network interface or adapter 170. When used in a WAN networking environment, the computer 110 typically includes a modem 172 or other means for establishing communications over the WAN 173, such as the Internet. The modem 172, which may be internal or external, may be connected to the system bus 121 via the user input interface 160, or other appropriate mechanism. In a networked environment, program modules depicted relative to the computer 110, or portions thereof, may be stored in the remote memory storage device. By way of example, and not limitation, FIG. 1 illustrates remote application programs 185 as residing on memory device 181. It will be appreciated that the network connections shown are exemplary and other means of establishing a communications link between the computers may be used.

Although the forgoing text sets forth a detailed description of numerous different embodiments of the invention, it should be understood that the scope of the invention is defined by the words of the claims set forth at the end of this patent. The detailed description is to be construed as exemplary only and does not describe every possibly embodiment of the invention because describing every possible embodiment would be impractical, if not impossible. Numerous alternative embodiments could be implemented, using either current technology or technology developed after the filing date of this patent, which would still fall within the scope of the claims defining the invention.

While the risk evaluation system and method, and other elements, have been described as preferably being implemented in software, they may be implemented in hardware, firmware, etc., and may be implemented by any other processor. Thus, the elements described herein may be implemented in a standard multi-purpose CPU or on specifically designed hardware or firmware such as an application-specific integrated circuit (ASIC) or other hard-wired device as desired, including, but not limited to, the computer 110 of FIG. 1. When implemented in software, the software routine may be stored in any computer readable memory such as on a magnetic disk, a laser disk, or other storage medium, in a RAM or ROM of a computer or processor, in any database, etc. Likewise, this software may be delivered to a user or a diagnostic system via any known or desired delivery method including, for example, on a computer readable disk or other transportable computer storage mechanism or over a communication channel such as a telephone line, the internet, wireless communication, etc. (which are viewed as being the same as or interchangeable with providing such software via a transportable storage medium).

Thus, many modifications and variations may be made in the techniques and structures described and illustrated herein without departing from the spirit and scope of the present invention. Thus, it should be understood that the methods and apparatus described herein are illustrative only and are not limiting upon the scope of the invention.

In one embodiment, the invention provides an apparatus for determining corrected PSA quantity in a human individual, comprising (a) a processor; and (b) a computer readable memory having computer executable instructions adapted to be executed on the processor, wherein said instructions comprise steps of (i) obtaining data representing uncorrected PSA quantity in a biological sample from the human individual; (ii) obtaining sequence data about at least one polymorphic marker in the genome of the human individual, wherein different alleles of the at least one polymorphic marker are predictive of different PSA quantity in humans; (iii) determining a corrected PSA quantity based on the sequence data about the at least one polymorphic marker. In one embodiment, the at least one allele of the at least one marker is predictive of an increased quantity of PSA in humans, and wherein at least one other allele of the at least one marker is predictive of a decreased quantity of PSA in humans.

Also provided is a computer-readable medium having computer executable instructions for determining corrected values of PSA quantity, the computer readable medium comprising (i) data indicative uncorrected values of PSA quantity for at least one human individual; (ii) data comprising sequence data about at least one polymorphic marker in the genome of the at least one human individual, wherein said at least polymorphic marker is predictive of PSA quantity in humans; and (iii) a routine stored on the computer readable medium and adapted to be executed by a processor to determine corrected PSA values for the at least one human individual.

Preferably, the markers useful in the computer-implemented functions described herein are selected from the group consisting of rs7193343, rs7618072, rs10077199, rs10490066, rs10516002, rs10519674, rs1394796, rs2935888, rs4560443, rs6010770 and rs7733337, and markers in linkage disequilibrium therewith.

The present invention will now be exemplified by the following non-limiting examples.

### Example 1

A genome-wide association study (GWAS) to search for sequence variants affecting population variation in PSA levels was performed, and the effects of PSA variants on subsequent prostate cancer diagnoses was investigated.

### Results

Sequence Variants Associated with PSA Levels

We performed a GWAS on PSA levels, adjusted for age and laboratory center, in Icelandic men not diagnosed with prostate cancer according to data from the nation-wide Icelandic Cancer Registry (ICR) until end of 2008. These men had also not undergone transurethral resection of the prostate (TURP), based on records from the Landspitali-National Hospital where 90% of all TURP procedures in the country are performed. In total, we had access to PSA measurements from 4,620 individuals genotyped on Illumina chips, containing either the 317K or the 370K HumanHap SNP panel. The analysis was augmented with data from 9,218 Icelanders with PSA measurements whose genetic information could be partially inferred from genotyped relatives (in-silico genotyping), using a previously described method (21-23). With respect to statistical power, this augmentation is equivalent to an additional 2,918 individuals on average (for details about the populations see Table 2). After quality control, 304,070 SNPs were available for the GWAS. Since the mean of the χ² values was below 1 (χ²=0.91) we did not apply any genomic control correction.

We selected all association signals with P<1×10⁻⁵ for further analysis. This represented 12 SNPs at 6 different loci, of which four loci reached genome-wide significance after accounting for the number of tests performed (P<1.64×10⁻⁷=0.05/304,070) (Table 3a). The genome-wide significant association signals were in or near genes at the following loci: KLK3 on 19q13.33; HNF1B on 17q12; FGFR2 on 10q26.12; and TBX3 on 12q24.21. The two suggestive association signals were at 10q11.23 near the MSMB gene and at 5p15.33 near the TERT gene (Table 3a).

To further investigate each of the six loci, we imputed genotypes based on data for 2.5M SNPs from the HapMap CEU individuals for all SNPs present within a window of 500 Kb centered on the most significant SNP. Based on this analysis, we identified three additional SNPs; rs2736098-A at 5p15.33, rs4430796-A at 17q12 and rs17632542-T at 19q13.33, that had stronger association effect on PSA levels than any SNP present on the 317K chip (Table 3b).

In an attempt to follow-up the observed associations with PSA levels in the Icelandic discovery group, we genotyped the most significant SNP at each of the six loci in an additional 1,919 Icelandic men with PSA level measurements and not diagnosed with prostate cancer, and in 454 men from the UK with PSA levels below 3 ng/ml and not diagnosed with prostate cancer. All UK participants in the present study came from the ProtecT trial (24). After combining significance levels from Iceland and the UK, at least one SNP at each locus reached genome-wide significance (Table 4).

For the strongest variant at each locus, the allele frequency was comparable in the Icelandic and UK populations with frequencies ranging from 24% to 93% (Table 4) and their observed effect on the PSA level ranges from 7% to 39% per allele in the Icelandic samples and from 5% to 102% per allele in the UK samples (see Table 4 and Table 5 for genotype effect of the variants.).

The strongest overall association effect observed in the present study is for two SNPs, rs2735839 and rs17632542, located near or in the PSA coding gene KLK3 (Table 4), of which rs2735839-G (and highly correlated markers) has previously been reported to associate with PSA levels (18-20, 26). The two SNPs are moderately correlated with each other (D′=1 and r²=0.48 in UK; r²=0.56 in Iceland; r²=0.56 in HapMap CEU phase 3).

When we adjusted the results for each SNP, using the other SNP as a covariate and only including individuals genotyped for both markers, results for rs17632542 remain significant after adjusting for rs2735839 (P_{combined}=5.51×10⁻⁸) whereas rs2735839 was marginally significant after adjusting for rs17632542 (P_{combined}=0.043). This suggests that the signal from rs2735839 is subsumed by rs17632542. The SNP rs17632542 is a missense mutation (an amino acid change denoted as 1179T) in KLK3. This amino acid alteration is defined as either neutral or deleterious by different online protein structure algorithms (see Table 6). A deleterious mutation could conceivably destabilize the protein, affecting circulating PSA levels. Alternatively, the mutation might affect the antigenicity of the protein and thereby influence its detectability in PSA tests. For the 10q11 (MSMB) and 17q12 (HNF1B) PSA loci, the alleles identified here i.e. rs10993994-T and rs4430796-A are the same as those previously reported to associate with PSA levels (25) as well as with prostate cancer risk (25, 27).

At the novel PSA locus on 10q26, two variants, rs10788160-A and rs12413088-T, were genome-wide significant and had similar effects on PSA levels. The two variants are located within an LD-region not known to contain any genes, 324 and 305 Kb centromeric to the start of the FGFR2 gene, respectively. The two variants are highly correlated (r²=0.85 in Iceland and r²=0.83 in the UK) and neither remains significant after adjusting for the other. Since the effects of the two variants cannot be distinguished from each other, we elected to focus on rs10788160-A in subsequent investigations. Sequence variants at the FGFR2 locus (rs1219648 and its surrogates) have been reported to predispose to breast cancer (28-30). The PSA variant, rs10788160, is in very low linkage disequilibrium with the variant conferring risk of breast cancer (D′=0.15, r²=0.01 between rs1219648 and rs10788160 in Iceland). No association was detected between rs10788160 and breast cancer in a case control study in Iceland (OR=0.97, P=0.36), or between rs1219648 and PSA levels in the GWAS of PSA (P=0.46). Hence, the variants at the FGFR2 locus conferring risk of breast cancer and variation in PSA levels seem to be distinct.

The most significant variant on 12q24, the second novel PSA locus, is rs11067228-A. This SNP is located in an LD-block that contains the gene TBX3 in which mutations have been found to cause the ulnar-mammary syndrome (OMIM #181450) but not previously shown to affect PSA levels.

At the third novel PSA locus, 5p15 near the TERT gene, two sequence variants, rs401681-C and rs2736098-A, were demonstrated to have a comparable effect on PSA levels. They are moderately correlated (D′=0.93 and r²=0.39 between rs401681 and rs2736098 according to HapMap CEU Phase 2), and because the effects of the variants cannot be distinguished from each other, we elected to focus on rs2736098-A in subsequent analyses.

We estimated the fraction of the total variance in the level of PSA explained by combining the effect from the best marker at each of the six loci (rs2736098, rs10993994, rs10788160, rs11067228, rs4430796 and rs17632542). The fraction accounted for is estimated to be 4.2% in Iceland and 11.8% in the UK. In both populations, the missense mutation in the KLK3 gene, rs17632542, accounts for half of the fraction of variance explained.

### The PSA Variants and Predisposition to Prostate Cancer

Variants at four of the six loci discussed above (KLK3, TERT, MSMB and HNF1B) have previously been reported to associate with risk of prostate cancer, although at different degrees of significance (18, 22, 25-27, 31) and some even with conflicting evidence (19). Due to the potential confounding effects of PSA levels and prostate cancer, we examined if the PSA SNPs identified in this study also associate with prostate cancer. Based on a combined analysis of over 5,325 prostate cancer cases and 41,417 controls from Iceland, the Netherlands, Spain, Romania and the US, we replicated the four loci previously reported to predispose to prostate cancer, each with a similar effect as described before (ORs ranging from 1.10 to 1.21; see Table 7). Interestingly, in our data the missense variant in KLK3, rs17632542, shows a stronger association with prostate cancer than the strongest previously reported variant at this locus, rs2735839 (OR=1.39 and 1.19 for rs17632542-T and rs2735839-G, respectively; see Table 7). In contrast, we found that neither of the variants at two of the three new PSA loci (FGFR2 and TBX3) associate significantly with prostate cancer (P_{combined}=0.27 and 0.54; OR_{combined}=0.97 and 1.01, for rs10788160-A and rs11067228, respectively).

We next examined if any of the six loci associated with PSA levels have an effect on age at diagnosis or aggressiveness of prostate cancer among patients in the 6 study groups, coming from Iceland, the Netherlands, Spain, Romania, the US and the UK. Only the missense mutation in KLK3, rs17632542, is significantly associated with age at diagnosis; for each allele of rs17632542-T, which associates with higher PSA levels, the age at diagnosis was estimated to decrease by ˜9 months (0.71 year decrease, P=0.016; see Table 8). When performing a case-only analysis, we observe that for the missense mutation in KLK3, rs17632542-T, the allele conferring risk of prostate cancer is significantly less frequent (OR=0.78, P=0.0099) among cases with more aggressive prostate cancer (Gleason score >6, and/or T3 or higher, and/or node positive, and/or with metastatic disease) compared to cases with less aggressive prostate cancer (Gleason score <7, and T2 or lower). This is in agreement with findings previously reported for the correlated variant at this locus, rs2735839(32, 33). For none of the five variants was a significant effect on the aggressiveness of the disease detected.

As discussed above, there has been some controversy in the literature about whether the predisposition to prostate cancer observed for the previously reported KLK3 variant (rs2735839) is mainly due to its strong effect on PSA levels and therefore, driven by the increasing frequency of PSA testing in the last decades (19, 20). In order to test for this, we stratified our Icelandic study group into cases diagnosed before 1992, a time when the majority of patients were diagnosed without undergoing PSA testing, and cases diagnosed from 1992 to 2008, a period in which PSA testing has become increasingly more frequent. We use in-silico genotyping based on familial imputation to augment the effective sample size of the group of cases, while we used 34,124 Icelanders not known to have prostate cancer as controls. Our results for rs2735839-G show that the association effect observed for the total case study group (OR=1.15 (95% CI 1.04-1.27), P=0.007) is confined to the group of cases diagnosed 1992 or later (OR=1.17 (95% CI. 1.06-1.29), P=0.002) whereas cases diagnosed before 1992 have no increased risk (OR=0.97 (95% CI. 0.83-1.13), P=0.7). These results support the notion that the prostate cancer risk reported for the KLK3 locus is driven by the increasing frequency of PSA testing and subsequent biopsies over the last few decades. In contrast, the results for the other three PSA loci that associate with increased risk of prostate cancer (TERT, HNF1B and MSNB) are not substantially different for the two case subgroups, diagnosed before or after 1992. As expected no effect on prostate cancer risk was observed in either group of cases for the FGFR2 and TBX3 SNPs.

### Effect of Prostate Cancer Risk Variants on PSA Levels

Due to the effect of prostate cancer on the level of PSA and the increased probability of being diagnosed with prostate cancer, given an increase in PSA levels, we assessed the effect on PSA levels of the 47 sequence variants conferring risk of prostate cancer reported to date (see Table 9) (selected SNPs based on the NIH Catalog of Published Genome-Wide Association Studies; http://www.genome.gov/26525384#1). Some loci have more than one reported SNP. According to our results, there is a clear tendency for the allele associated with prostate cancer risk also to be associated with high levels of PSA (see Table 9). This is comparable to results previously reported by Wiklund et al.(20). For the vast majority of the loci (N=41), their effect on PSA level is weak (well below 0.1 standard unit) and likely reflects undiagnosed prostate cancer cases in the PSA study group (also suggested by Wiklund et al 2008(20)). Exceptions are the variants at the KLK3 (rs2735839 and rs17632542), HNF1B (rs4430769), MSMB (rs10993994) and the TERT loci (rs2736098), the loci of genome-wide significance in our PSA GWA study. Variants at two other loci 11q13 (rs11228565) and 8q24 (rs16901979) also have greater effects on PSA levels but the effects did not reach genome-wide significance levels. These six loci can roughly be divided into two groups: those with a moderate effect on the PSA levels compared to their effect on prostate cancer risk (8q24, 11q13, 10q11 and 17q12) and those comprised of variants that have a relatively strong PSA effect compared to their effect on prostate cancer risk (i.e. variants at: KLK3 on 19q13.33, and TERT on 5p15).

### Sequence Variants and Benign Prostatic Hyperplasia

Benign prostatic hyperplasia (BPH) can affect PSA levels. In order to determine if any of the PSA variants discussed above are associated with BPH, we used a set of 33,779 Icelandic controls and 2,312 Icelandic men with BPH; defined as individuals either diagnosed after undergoing TURP or men over the age of 50 repeatedly using drugs in the G04C group of the ATC classification (e.g. Tamsulosin, Finasteride and Dutasteride) between the years2003 and 2009 (see Methods). Except for rs2736098-T on 5p15 that showed a nominally significant association (P=0.048, OR=1.08), no association was observed between BPH and any of the remaining five PSA variants, given the number of tests performed. Hence, BPH is unlikely to account for a significant fraction of the observed association with PSA levels for the variants discussed here.

### PSA Sequence Variants and Prostate Biopsies

When screening for prostate cancer, a PSA level above a certain cutoff value is considered an indication for performing a needle biopsy. We wanted to assess if the variants that associate with increased PSA levels also make men more prone to undergo a biopsy of the prostate. In our study group of 2,300 Icelandic men who underwent a prostate biopsy between 1998 and 2008, we observed a higher frequency of the allele increasing PSA-levels in those undergoing biopsies than in population controls for all six variants (1.04≦OR≦1.46; all SNPs have P<0.05 except rs11067228 on 12q24 which has P=0.25, see Table 10). Among the 2,300 individuals who had undergone a biopsy, cancer had been diagnosed in close to 50% (a positive biopsy). When restricting the analysis to individuals with biopsy but no detectable prostate cancer (negative biopsy) and comparing them to population controls, similar or even stronger results were observed (1.03≦OR≦1.82; all SNPs have P<0.05 except rs10993994 near MSMB which has P=0.48, see Table 11). From the UK study group, we had access to a group of approximately 1,400 men who had undergone a biopsy. Of those, about one third was diagnosed with prostate cancer. Using the Icelandic and the UK study groups of men who had been biopsied, we compared the frequency of the PSA variants in positive and negative biopsies. Of the six loci we found that for the three PSA variants not primarily associated with prostate cancer risk (KLK3, FGFR2 and TBX3), the PSA increasing allele was significantly less frequent among men with a positive biopsy than in men with a negative biopsy (rs10788160-A near FGFR2 has OR_{combined}=0.79 and P_{combined}=5.4×10⁻⁶, rs11067228-A near TBX3 has OR_{combined}=0.87 and P_{combined}=0.0034, rs17632542-T in KLK3 has OR_{combined}=0.77 and P_{combined}=0.013; see Table 12). The results for these three variants demonstrate that the alleles associated with increased PSA level increase the probability that a normal prostate is biopsied.

### Discussion

In this study, we identified 6 loci that associate with PSA levels with genome-wide significance. Variants at three of these loci had previously been shown to associate with PSA levels whereas three of the loci, at 10q26, 5p15 and 12q24, are novel. Unlike the variants previously reported to associate with PSA levels, two of the novel loci, i.e. 12q24 and 10q26, do not associate with prostate cancer risk and the third locus, at 5p15, has only a moderate effect on prostate cancer. Furthermore, we have shown that two of these variants (rs10788160-A on 10q26 and rs11067228-A on 12q24), together with the KLK3 variant, are associated with a greater probability of having a normal prostate biopsied. Hence, these new markers primarily predict the outcome of the PSA-based prostate cancer screening process, i.e. the decision of performing a biopsy or not, and the outcome of the biopsy, rather than predisposition to prostate cancer.

In our study we showed that a missense mutation, rs17632542-T, in the KLK3 gene on 19q33.33 is associated with higher PSA levels. This variant has a stronger effect on PSA than the variant rs2735839, previously reported at this locus. The KLK3 variant was also found to predispose to prostate cancer but the association effect was confined to the group of cases primarily diagnosed after the introduction of the PSA test. Furthermore, the association with prostate cancer at the KLK3 locus was shown to be predominantly with the less aggressive form of the disease. We have also shown that, given biopsy, the variant rs17632542-T is associated with greater probability of not being diagnosed with cancer. Together, these results suggest that the reported association with prostate cancer at the KLK3 locus is mainly driven by its effect on PSA levels and the increasing frequency of PSA testing in men.

### REFERENCES

1. Jemal, A., et al. M. J. Cancer statistics, 2009. CA Cancer J Clin, 59: 225-49, 2009.
2. Barry, M. J. Screening for prostate cancer—the controversy that refuses to die. N Engl J Med, 360: 1351-4, 2009.
3. Nam, R. K., et al. Utility of incorporating genetic variants for the early detection of prostate cancer. Clin Cancer Res, 15: 1787-93, 2009.
4. Thompson, I. M., et al. Assessing prostate cancer risk: results from the Prostate Cancer Prevention Trial. J Natl Cancer Inst, 98: 529-34, 2006.
5. Bradford, T. J., et al. Molecular markers of prostate cancer. Urol Oncol, 24: 538-51, 2006.
6. Vickers, A. J., et al. Prostate-Specific Antigen Velocity for Early Detection of Prostate Cancer: Result from a Large, Representative, Population-based Cohort. Eur Urol, 2009.
7. Schroder, F. H., et al. Screening and prostate-cancer mortality in a randomized European study. N Engl J Med, 360: 1320-8, 2009.
8. Andriole, G. L., et al. Mortality results from a randomized prostate-cancer screening trial. N Engl J Med, 360: 1310-9, 2009.
9. van Leeuwen, P. J., et al. Prostate cancer mortality in screen and clinically detected prostate cancer: estimating the screening benefit. Eur J Cancer, 46: 377-83.
10. Hugosson, J., et al. Mortality results from the Goteborg randomised population-based prostate-cancer screening trial. Lancet Oncol.
11. Neal, D. E. PSA testing for prostate cancer improves survival—but can we do better? Lancet Oncol, 2010.
12. Thompson, I. M., et al. Operating characteristics of prostate-specific antigen in men with an initial PSA level of 3.0 ng/ml or lower. Jama, 294: 66-70, 2005.
13. Oesterling, J. E., et al. Serum prostate-specific antigen in a community-based population of healthy men. Establishment of age-specific reference ranges. Jama, 270: 860-4, 1993.
14. DeAntoni, E. P., et al. Age- and race-specific reference ranges for prostate-specific antigen from a large community-based study. Urology, 48: 234-9, 1996.
15. Emilsson, V., et al. Genetics of gene expression and its effect on disease. Nature, 452: 423-8, 2008.
16. Bansal, A., et al. Heritability of prostate-specific antigen and relationship with zonal prostate volumes in aging twins. J Clin Endocrinol Metab, 85: 1272-6, 2000.
17. Pilia, G., et al. Heritability of cardiovascular and personality traits in 6,148 Sardinians. PLoS Genet, 2: e132, 2006.
18. Eeles, R. A., et al. Multiple newly identified loci associated with prostate cancer susceptibility. Nat Genet, 40: 316-21, 2008.
19. Ahn, J., et al. Variation in KLK genes, prostate-specific antigen and risk of prostate cancer. Nat Genet, 40: 1032-4; author reply 1035-6, 2008.
20. Wiklund, F., et al. Association of reported prostate cancer risk alleles with PSA levels among men without a diagnosis of prostate cancer. Prostate, 69: 419-27, 2009.
21. Gudbjartsson, D. F., et al. Many sequence variants affecting diversity of adult human height. Nat Genet, 40: 609-15, 2008.
22. Rafnar, T., et al. Sequence variants at the TERT-CLPTM1L locus associate with many cancer types. Nat Genet, 41: 221-7, 2009.
23. Gudmundsson, J., et al. Common variants on 9q22.33 and 14q13.3 predispose to thyroid cancer in European populations. Nat Genet, 41: 460-4, 2009.
24. Moore, A. L., et al. Population-based prostate-specific antigen testing in the UK leads to a stage migration of prostate cancer. BJU Int, 104: 1592-8, 2009.
25. Thomas, G., et al. Multiple loci identified in a genome-wide association study of prostate cancer. Nat Genet, 40: 310-5, 2008.
26. Pal, P., et al. Tagging SNPs in the kallikrein genes 3 and 2 on 19q13 and their associations with prostate cancer in men of European origin. Hum Genet, 122: 251-9, 2007.
27. Gudmundsson, J., et al. Two variants on chromosome 17 confer prostate cancer risk, and the one in TCF2 protects against type 2 diabetes. Nat Genet, 39: 977-83, 2007.
28. Hunter, D. J., et al. A genome-wide association study identifies alleles in FGFR2 associated with risk of sporadic postmenopausal breast cancer. Nat Genet, 39: 870-4, 2007.
29. Easton, D. F., et al. Genome-wide association study identifies novel breast cancer susceptibility loci. Nature, 447: 1087-93, 2007.
30. Stacey, S. N., et al. Common variants on chromosome 5p12 confer susceptibility to estrogen receptor-positive breast cancer. Nat Genet, 40: 703-6, 2008.
31. Kote-Jarai, Z., et al. Multiple novel prostate cancer predisposition loci confirmed by an international study: the PRACTICAL Consortium. Cancer Epidemiol Biomarkers Prev, 17: 2052-61, 2008.
32. Xu, J., et al. Association of prostate cancer risk variants with clinicopathologic characteristics of the disease. Clin Cancer Res, 14: 5819-24, 2008.
33. Kader, A. K., et al. Individual and cumulative effect of prostate cancer risk-associated variants on clinicopathologic variables in 5,895 prostate cancer patients. Prostate, 69: 1195-205, 2009.
34. Gulcher, J. R., et al. Protection of privacy by third-party encryption in genetic research in Iceland. Eur J Hum Genet, 8: 739-42, 2000.
35. Gretarsdottir, S., et al. The gene encoding phosphodiesterase 4D confers risk of ischemic stroke. Nat Genet, 35: 131-8, 2003.

TABLE 2
Characteristics of men with PSA measurements in Iceland and UK used in the
analysis

| | | | Mean | | | |
|---|---|---|---|---|---|---|
| | | | age | | | |
| | | | (years) | Mean | | |
| | | | at | number | Median PSA- | |
| Study | Sub- | Individuals | PSA | of PSA- | value (ng/ml) | Recruitment |
| group | classification | (n) | (s.d.) | measurements | (1st_quartile, 3rd_quartile) | period |
| Iceland | Chip-genotyped | 4,620 | 66 (12) | 2.8 | 1.69 (0.87, 3.6) | 1994-2009 |
| | individuals | | | | | |
| | Used for in- | 9,218 | 60 (13) | 2.1 | 1.50 (0.80, 3.2) | 1994-2009 |
| | silico | | | | | |
| | genotyping | | | | | |
| | Single track | 1,919 | 63 (12) | 2.8 | 2.90 (0.73, 6.3) | 1994-2009 |
| | assay | | | | | |
| | genotyping | | | | | |
| | Total | 15,757 | | | | |
| UK | All with single | | | | | |
| | track assay | | | | | |
| | genotyping: | | | | | |
| | PSA below 3 ng/ml | 454 | 63 (5) | 1 | 1.50 (0.70, 2.20) | 1999-2007 |
| | PSA from 3-10 ng/ml | 960 | 62 (5) | 1 | 4.10 (3.50, 5.07) | 1999-2007 |
| | and | | | | | |
| | biopsy negative | | | | | |
| | PSA >3 ng/ml | 523 | 63 (5) | 1 | 6.00 (3.90, 14.0) | 1999-2007 |
| | and biopsy | | | | | |
| | positive | | | | | |
| | Total | 1,937 | | | | |

Shown are the relevant characteristics for the Icelandic and United Kingdom (UK) study groups;
number (n) of individuals in each sup-group, the mean age (years) at the first PSA level measurement and the standard deviation (s.d.), the mean number of PSA measurements for each sub-study group, the median PSA value (ng/ml) and the recruitment period.

TABLE 3
Association results from the GWAS on PSA levels in Iceland

| | | | | | | | |
|---|---|---|---|---|---|---|---|
| | | | Closest | Position | Individuals | Allele | |
| SNP | Allele | Locus | gene | (bp) | (n) | Frequency | P-value |

a. Results for SNPs present on the Illumina 317K SNP chip

| | | | | | | | | |
|---|---|---|---|---|---|---|---|---|
| | | | | | | | Assoc. | |
| | | | | | | | effect | |
| | | | | | | | (%) | |
| rs401681 | C | 5p15.33 | TERT | 1,375,087 | 7,508 | 0.55 | 6.9 | 5.7E−06 |
| rs10993994 | T | 10q11.23 | MSMB | 51,219,502 | 7,507 | 0.39 | 7.2 | 5.8E−06 |
| rs10788160 | A | 10q26.12 | FGFR2 | 123,023,539 | 7,322 | 0.31 | 9.2 | 1.1E−07 |
| rs12413088 | T | 10q26.12 | FGFR2 | 123,042,718 | 7,656 | 0.28 | 8.0 | 3.0E−06 |
| rs11067228 | A | 12q24.21 | TBX3 | 113,578,643 | 7,564 | 0.56 | 8.3 | 1.5E−07 |
| rs3744763 | C | 17q12 | HNF1B | 33,164,998 | 7,392 | 0.60 | 8.4 | 6.5E−08 |
| rs7501939 | C | 17q12 | HNF1B | 33,175,269 | 7,432 | 0.58 | 7.9 | 5.3E−07 |
| rs266849 | A | 19q13.33 | KLK3 | 56,040,902 | 7,643 | 0.83 | 16.1 | 1.2E−13 |
| rs266870 | T | 19q13.33 | KLK3 | 56,043,746 | 7,583 | 0.51 | 9.7 | 1.3E−09 |
| rs1058205 | T | 19q13.33 | KLK3 | 56,055,210 | 7,575 | 0.82 | 19.4 | 5.4E−20 |
| rs2735839 | G | 19q13.33 | KLK3 | 56,056,435 | 7,533 | 0.87 | 22.5 | 1.8E−21 |
| rs1506684 | T | 19q13.33 | KLK3 | 56,063,231 | 7,487 | 0.58 | 9.3 | 1.9E−09 |

b. Imputed results for SNPs not present on the Illumina 317K SNP chip

| | | | | | | | | |
|---|---|---|---|---|---|---|---|---|
| | | | | | | | Association | |
| | | | | | | | effect | |
| | | | | | | | (%) | |
| rs2736098 | A | 5p15.33 | TERT | 1,347,086 | 4,506 | 0.33 | 11.5 | 8.8E−07 |
| rs4430796 | A | 17q12 | HNF1B | 33,172,153 | 4,506 | 0.52 | 11.3 | 3.8E−09 |
| rs17632542 | T | 19q13.33 | KLK3 | 56,053,569 | 4,506 | 0.91 | 35.7 | 1.6E−18 |

Part a) of the table: shown are genome-wide association results for SNPs with P < 1E−05, the number of individuals (n) with PSA measurement and either genotyped using the Illumina 317K chip (on average 4,599 men) or by the in-silico genotyping method (on average 2,918 men), the allele associated with increased PSA levels, the association effect per allele and the two-sided P-value.
Part b) of the table: shown are association results for the three SNPs that showed a stronger effect than the chip-genotyped SNPs. The imputation analysis was based on 2.5M HapMap SNPs, testing all SNPs within a window of 500 Kb for all six loci shown in section a) of this table.

TABLE 4
Association results for SNPs and PSA levels, based on samples from Iceland and UK.

| | | | | Iceland | | | | UK | | | | |
|---|---|---|---|---|---|---|---|---|---|---|---|---|
| | | | | | | | Increase | | | | Increase | |
| | | | | | | | per | | | | per | |
| SNP | | | | | | Total | allele | | | | allele | Combined |
| (SEQ ID NO) | Allele | Chr | Position (bp) | P-value | Freq. | (n) | (%) | P-value | Freq. | Total (n) | (%) | P-value |
| rs401681 (1) | C | 5 | 1,375,087 | 1.88E−09 | 0.55 | 9,049 | 7 | 0.002 | 0.53 | 451 | 19 | 1.20E−10 |
| rs2736098* (2) | A | 5 | 1,347,086 | 5.10E−10 | 0.33 | 6,347 | 10.5 | 0.021 | 0.27 | 450 | 14.8 | 2.84E−10 |
| rs10788160 (3) | A | 10 | 123,023,539 | 8.88E−14 | 0.31 | 8,686 | 10.2 | 0.0012 | 0.24 | 453 | 22.9 | 4.50E−15 |
| rs10993994 (4) | T | 10 | 51,219,502 | 9.25E−14 | 0.39 | 8,870 | 9.2 | 0.46 | 0.38 | 453 | 5.4 | 6.66E−13 |
| rs11067228 (5) | A | 12 | 113,578,643 | 1.09E−11 | 0.56 | 8,882 | 8.3 | 0.074 | 0.56 | 441 | 9.2 | 1.93E−11 |
| rs4430796* (6) | A | 17 | 33,172,153 | 1.40E−11 | 0.52 | 6,222 | 9.4 | 0.21 | 0.5 | 449 | 6.3 | 5.60E−11 |
| rs2735839 (7) | G | 19 | 56,056,435 | 4.84E−43 | 0.87 | 8,869 | 25.4 | 1.18E−06 | 0.86 | 445 | 49.7 | 6.26E−47 |
| rs17632542* | T | 19 | 56,053,569 | 9.00E−40 | 0.91 | 6,078 | 39.1 | 2.66E−09 | 0.93 | 435 | 102.2 | 3.05E−46 |
| (8) | | | | | | | | | | | | |

Shown are results for alleles that associate with increased (%) levels of PSA. Results for SNPs present on the Illumina chips are based on genotypes from chip (~50%), in-silico genotyping using family imputation (~30%), and single track assay genotyping (~20%)
*These SNPs (rs273098, rs4430796, and rs17632542) are not on the Illumina chips used in the present study and results are based on genotypes from HapMap SNP imputation (~70%) and single track assay (~30%) genotyping.

TABLE 5
Estimates from Iceland and UK on the relative genotype effect for SNPs associated with PSA levels

| | | | | | | | | | | | |
|---|---|---|---|---|---|---|---|---|---|---|---|
| | | | | Allelic | Relative | XX | XX relative | OX | OX relative | OO | OO relative |
| SNP | Allele | Chr | Position (bp) | Frequency | Allelic effect | Frequency | gt-effect | Frequency | gt-effect | Frequency | gt-effect |

a. Results for the Icelandic study group

| | | | | | | | | | | | |
|---|---|---|---|---|---|---|---|---|---|---|---|
| rs2736098 | A | 5 | 1,347,086 | 0.33 | 1.11 | 0.11 | 1.14 | 0.44 | 1.03 | 0.45 | 0.93 |
| rs401681 | C | 5 | 1,375,087 | 0.55 | 1.07 | 0.3 | 1.06 | 0.5 | 0.99 | 0.2 | 0.93 |
| rs10993994 | T | 10 | 51,219,502 | 0.39 | 1.09 | 0.15 | 1.11 | 0.47 | 1.02 | 0.38 | 0.93 |
| rs10788160 | A | 10 | 123,023,539 | 0.31 | 1.1 | 0.1 | 1.14 | 0.43 | 1.04 | 0.48 | 0.94 |
| rs11067228 | A | 12 | 113,578,643 | 0.56 | 1.08 | 0.31 | 1.07 | 0.49 | 0.99 | 0.2 | 0.91 |
| rs4430796 | A | 17 | 33,172,153 | 0.52 | 1.09 | 0.27 | 1.09 | 0.5 | 0.99 | 0.23 | 0.91 |
| rs17632542 | T | 19 | 56,053,569 | 0.91 | 1.39 | 0.82 | 1.05 | 0.17 | 0.76 | 0.01 | 0.54 |
| rs2735839 | G | 19 | 56,056,435 | 0.87 | 1.25 | 0.75 | 1.06 | 0.23 | 0.84 | 0.02 | 0.67 |

b. Results for the UK study group

| | | | | | | | | | | | |
|---|---|---|---|---|---|---|---|---|---|---|---|
| rs2736098 | A | 5 | 1,347,086 | 0.27 | 1.15 | 0.07 | 1.22 | 0.39 | 1.06 | 0.53 | 0.92 |
| rs401681 | C | 5 | 1,375,087 | 0.53 | 1.19 | 0.29 | 1.17 | 0.5 | 0.98 | 0.22 | 0.82 |
| rs10993994 | T | 10 | 51,219,502 | 0.38 | 1.05 | 0.14 | 1.07 | 0.47 | 1.01 | 0.39 | 0.96 |
| rs10788160 | A | 10 | 123,023,539 | 0.24 | 1.23 | 0.06 | 1.36 | 0.37 | 1.1 | 0.57 | 0.9 |
| rs11067228 | A | 12 | 113,578,643 | 0.56 | 1.09 | 0.31 | 1.08 | 0.49 | 0.99 | 0.2 | 0.9 |
| rs4430796 | A | 17 | 33,172,153 | 0.5 | 1.06 | 0.25 | 1.06 | 0.5 | 1 | 0.25 | 0.94 |
| rs17632542 | T | 19 | 56,053,569 | 0.93 | 2.02 | 0.86 | 1.08 | 0.14 | 0.53 | 0.01 | 0.26 |
| rs2735839 | G | 19 | 56,056,435 | 0.86 | 1.5 | 0.73 | 1.1 | 0.25 | 0.74 | 0.02 | 0.49 |

Shown are the SNPs and their alleles associated with increasing PSA levels and the genotype (gt) frequency and the relative genotype (gt) effect on PSA levels, compared to the average of the population under study: for homozygous (XX), heterozygous (OX), and non-carriers (OO) of the allele associated with elevated PSA levels.

TABLE 6
Bioinformatic analysis of the KLK3
missense variant rs17632542 (I179T)

| | | Nonsynonymous |
|---|---|---|
| | | (I179T); change from |
| | | medium size and |
| | | hydrophobic (I) to |
| | | medium size and |
| | Amino acid variation | polar (T) |
| Prediction Tool | Analysis Type | Prediction Results |
| PhastCons_44way^{a} | Conservation | not conserved |
| F-Score^{b} | Structure/Conservation | 0.75 |
| Panther subPSEC^{c} | Structure/Conservation | −6.28 |
| Panther Pdeleterious^{c} | Structure/Conservation | Probability of being |
| | | deleterious = 97% |
| PolyPhen^{d} | Structure/Conservation | benign |
| LS-SNP^{e} | Structure/Conservation | deleterious |
| SNPeffect^{f} | Structure/Conservation | deleterious |
| SNPs3D^{g} | Structure/Conservation | deleterious |
| ESEfinder^{h} | Exonic splicing enhancer | changed |
| ESRSearchⁱ | Exonic splicing enhancer | changed |
| PESX^{j} | Exonic splicing enhancer | changed |
| RESCUE_ESE^{k} | Exonic splicing enhancer | not changed |

^{a}Carries out multiple alignments of 44 vertebrate species and returns measures of evolutionary conservation using a phylogenetic hidden Markov model (phylo-HMM). Siepel A, et al., Genome Res 15: 1034-1050, 2005.
^{b}Uses the F-SNP database (http://compbio.cs.queensu.ca/F-SNP/) to provide integrated information about the functional effects of SNPs obtained from 16 different bioinformatic tools and databases. Functional effects are predicted and indicated at the splicing, transcriptional, translational and post-translational levels.
^{c}Panther estimates the likelihood of a particular nsSNP to cause a functional impact on the protein. It calculates subPSEC (substitution position-specific evolutionary conservation) score based on an alignment of evolutionarily related proteins. It then calculates Pdeleterious, the probability that a given variant will have a deleterious effect on protein function, such that a subPSEC score of −3 corresponds to a Pdeleterious of 0.5. Brunham L R, et al. PLoS Genet 1(6) 2005: e83. doi: 10.1371/journal.pgen.0010083.
^{d}PolyPhen predicts the possible impact of an amino acid substitution on the structure and function of a human protein using straightforward physical and comparative considerations. Ramensky, V, et al. Nucleic Acids Res 30(17): 3894-900, 2002.
^{e}Disease-associated nsSNPs are predicted by a support vector machine (SVM) trained on OMIM amino-acid variants and putatively neutral nsSNPs from dbSNP. Karchin R, et al. Bioinformatics 21(12): 2814-20, 2005.
^{f}The SNPeffect database uses sequence- and structure-based bioinformatics tools to predict the effect of non-synonymous SNPs on the molecular phenotype of proteins. Reumers J, et al., Bioinformatics 22: 2183-2185, 2006.
^{g}SNPs3D assigns molecular functional effects of non-synonymous SNPs based on structure and sequence analysis. Peng Y and John M, J Mol Biol. 356(5): 1263-74, 2006.
^{h}ESEfinder uses position weighted matrices to predict putative human exonic splicing enhancers (ESEs). Cartegni L, et al., Nucleic Acids Res 31(13): 3568-3571, 2003.
ⁱESRSearch uses the evolutionary conservation of wobble positions between human and mouse orthologous exons and the analysis of the overabundance of sequence motifs, compared with their random expectation, given by their codon relative frequency, to predict ESEs. Goren A, et al., Mol Cell. 22(6): 769-81, 2006.
^{j}PESX compares the frequency of all 65536 8-mers in internal non-coding exons against their adjacent pseudo exons and in internal non-coding exons against 5′UTR of intronless genes to predict ESEs. Zhang X H and Chasin L A, Genes Dev 18(11): 1241-1250, 2004.
^{k}Specific hexanucleotide sequences were identified as candidate ESEs on the basis that they have both significantly higher frequency of occurrence in exons than in introns and also significantly higher frequency in exons with weak (non-consensus) splice sites than in exons with strong (consensus) splice sites. Fairbrother W G, et al., Science 297(5583): 1007-13, 2002.

TABLE 7
Association of the six PSA SNPs with prostate cancer in Iceland, The Netherlands, Spain, Romania, and the US
a. Combined association results from a case-control association analysis in five study populations

| | | | Position | | Controls | Frequency | | | | |
|---|---|---|---|---|---|---|---|---|---|---|
| SNP | Allele | Chr | (bp) | Cases (n) | (n) | Cases | Controls | OR | P-value | Pₕₑₜ |
| rs2736098 | A | 5 | 1,347,086 | 5,009 | 41,334 | 0.3 | 0.29 | 1.11 | 3.50E−04 | 0.28 |
| rs10993994 | T | 10 | 51,219,502 | 5,077 | 41,168 | 0.45 | 0.4 | 1.21 | 7.70E−15 | 0.0066 |
| rs10788160 | A | 10 | 123,023,539 | 5,317 | 41,417 | 0.25 | 0.25 | 0.97 | 2.70E−01 | 0.65 |
| rs11067228 | A | 12 | 113,578,643 | 5,325 | 41,383 | 0.55 | 0.54 | 1.01 | 5.40E−01 | 0.16 |
| rs4430796 | A | 17 | 33,172,153 | 5,162 | 41,320 | 0.55 | 0.51 | 1.2 | 3.20E−13 | 0.29 |
| rs17632542 | T | 19 | 56,053,569 | 5,284 | 40,522 | 0.95 | 0.93 | 1.39 | 1.80E−10 | 0.052 |
| rs2735839 | G | 19 | 56,056,435 | 5,080 | 41,120 | 0.88 | 0.86 | 1.19 | 1.10E−06 | 0.89 |

b. Odds ratio and P-value for each study population from an case-control association analysis of prostate cancer

| SNP | OR_ICE | P_ICE | OR_NL | P_NL | OR_US | P_US | OR_ROM | P_ROM | OR_SPA | P_SPA |
|---|---|---|---|---|---|---|---|---|---|---|
| rs2736098 | 1.08 | 7.50E−02 | 1.17 | 1.20E−02 | 1.13 | 3.80E−02 | 0.83 | 2.00E−01 | 1.15 | 1.20E−01 |
| rs10993994 | 1.11 | 2.10E−03 | 1.2 | 1.20E−03 | 1.4 | 2.40E−10 | 1.17 | 2.80E−01 | 1.32 | 2.60E−04 |
| rs10788160 | 0.96 | 3.10E−01 | 0.98 | 7.50E−01 | 1.04 | 5.10E−01 | 0.92 | 6.30E−01 | 0.9 | 1.70E−01 |
| rs11067228 | 0.96 | 2.40E−01 | 1.01 | 8.50E−01 | 1.09 | 1.10E−01 | 0.98 | 9.50E−01 | 1.12 | 8.40E−02 |
| rs4430796 | 1.17 | 3.20E−05 | 1.26 | 5.00E−05 | 1.26 | 9.00E−06 | 1.3 | 5.90E−02 | 1.07 | 3.20E−01 |
| rs17632542 | 1.23 | 3.00E−03 | 1.61 | 1.80E−04 | 1.52 | 5.10E−04 | 1.16 | 6.10E−01 | 2.01 | 1.20E−04 |
| rs2735839 | 1.15 | 6.60E−03 | 1.25 | 4.00E−03 | 1.22 | 1.10E−02 | 1.09 | 6.90E−01 | 1.23 | 1.00E−01 |

Shown are: the allele associated with increased PSA levels, the number of cases and controls (n), the allele frequency in cases and controls, the odds ratio (OR) and the two-sided P-value. For the combined study populations the OR and P-values were estimated using the Mantel-Haenszel model.
Abbreviations for study populations are: Iceland (ICE), the Netherlands (NL), Chicago USA (US), Romania (ROM), and Spain (SPA).

TABLE 8
Effect of the allele conferring elevated PSA levels on age at diagnosis among
6,406 patients from six European ancestry study populations

| | Allele | | | | | | |
|---|---|---|---|---|---|---|---|
| | increasing | | | | | | |
| | PSA- | | Age effect | | | | |
| SNP | levels | Chromosome | (year) | 95% CI (year) | P_value | Pₕₑₜ | I₂ |
| rs2736098 | A | 5 | −0.23 | (−0.51, 0.06) | 0.13 | 0.0037 | 71.4 |
| rs10993994 | T | 10 | 0.19 | (−0.08, 0.45) | 0.17 | 0.76 | 0 |
| rs10788160 | A | 10 | 0.01 | (−0.10, 0.11) | 0.96 | 0.6 | 0 |
| rs11067228 | A | 12 | −0.10 | (−0.36, 0.17) | 0.48 | 0.86 | 0 |
| rs4430796 | A | 17 | −0.15 | (−0.41, 0.11) | 0.27 | 0.51 | 0 |
| rs17632542 | T | 19 | −0.71 | (−1.29, −0.13) | 0.016 | 0.2 | 31.3 |

Of the six PSA-associated SNPs, only the missense mutation in KLK3, rs17632542-T, is significantly associated with age at prostate cancer diagnosis.
The T allele of rs17632542, which associates with a higher PSA levels, is associated with a decrease in age at diagnosis of 9 months for each allele carried (−0.71 years).
Study populations:
Chicago, the US: 1578 patients
The Netherlands: 1088 patients
Iceland: 2258 patients
Romania: 309 patients
Spain: 656 patients
United Kingdom: 517 patients

TABLE 9
Association of the 47 previously reported prostate cancer risk SNPs with PSA
levels and prostate cancer in Iceland

| | | | | PSA | | | | Prostate cancer | | | |
|---|---|---|---|---|---|---|---|---|---|---|---|
| SNP | Allele | Chr. | Position (bp) | P-value | Effect s.u. | n | Freq. | P-value | OR | Cases (n) | Controls (n) |
| rs1465618 | C | 2 | 43,407,453 | 4.50E−01 | −0.01794 | 4,470 | 0.807 | 1.42E−01 | 0.94 | 1,757 | 36,145 |
| rs1465618 | T | 2 | 43,407,453 | 4.50E−01 | 0.017935 | 4,470 | 0.193 | 1.42E−01 | 1.06 | 1,757 | 36,145 |
| rs721048 | A | 2 | 62,985,235 | 5.58E−01 | −0.0137 | 4,506 | 0.201 | 5.16E−04 | 1.16 | 1,763 | 36,400 |
| rs721048 | G | 2 | 62,985,235 | 5.58E−01 | 0.013701 | 4,506 | 0.799 | 5.16E−04 | 0.87 | 1,763 | 36,400 |
| rs2710646 | A | 2 | 62,988,383 | 6.23E−01 | −0.0116 | 4,461 | 0.196 | 3.13E−04 | 1.16 | 1,745 | 36,061 |
| rs2710646 | C | 2 | 62,988,383 | 6.23E−01 | 0.011599 | 4,461 | 0.804 | 3.13E−04 | 0.86 | 1,745 | 36,061 |
| rs12621278 | A | 2 | 173,019,799 | 1.08E−01 | 0.065471 | 4,506 | 0.942 | 1.08E−02 | 1.22 | 1,763 | 36,400 |
| rs12621278 | G | 2 | 173,019,799 | 1.08E−01 | −0.06547 | 4,506 | 0.058 | 1.08E−02 | 0.82 | 1,763 | 36,400 |
| rs2660753 | C | 3 | 87,193,364 | 8.78E−01 | −0.0049 | 4,503 | 0.903 | 4.23E−02 | 0.89 | 1,761 | 36,349 |
| rs2660753 | T | 3 | 87,193,364 | 8.78E−01 | 0.004899 | 4,503 | 0.097 | 4.23E−02 | 1.12 | 1,761 | 36,349 |
| rs10934853 | A | 3 | 129,521,063 | 1.70E−02 | 0.050924 | 4,481 | 0.269 | 3.53E−03 | 1.12 | 1,754 | 36,151 |
| rs10934853 | C | 3 | 129,521,063 | 1.70E−02 | −0.05092 | 4,481 | 0.731 | 3.53E−03 | 0.89 | 1,754 | 36,151 |
| rs12500426 | A | 4 | 95,733,632 | 3.60E−01 | −0.01745 | 4,502 | 0.402 | 1.59E−01 | 1.05 | 1,762 | 36,356 |
| rs12500426 | C | 4 | 95,733,632 | 3.60E−01 | 0.017452 | 4,502 | 0.598 | 1.59E−01 | 0.95 | 1,762 | 36,356 |
| rs17021918 | C | 4 | 95,781,900 | 9.50E−01 | 0.001227 | 4,506 | 0.639 | 7.05E−01 | 1.01 | 1,763 | 36,400 |
| rs17021918 | T | 4 | 95,781,900 | 9.50E−01 | −0.00123 | 4,506 | 0.361 | 7.05E−01 | 0.99 | 1,763 | 36,400 |
| rs7679673 | A | 4 | 106,280,983 | 5.18E−01 | 0.012612 | 4,506 | 0.363 | 7.92E−03 | 0.91 | 1,763 | 36,400 |
| rs7679673 | C | 4 | 106,280,983 | 5.18E−01 | −0.01261 | 4,506 | 0.637 | 7.92E−03 | 1.1 | 1,763 | 36,400 |
| rs2736098 | C | 5 | 1,347,086 | 8.80E−07 | −0.12272 | 4,506 | 0.657 | 7.51E−02 | 0.92 | 1,763 | 36,400 |
| rs2736098 | T | 5 | 1,347,086 | 8.80E−07 | 0.122718 | 4,506 | 0.343 | 7.51E−02 | 1.08 | 1,763 | 36,400 |
| rs401681 | C | 5 | 1,375,087 | 7.46E−04 | 0.063589 | 4,502 | 0.545 | 5.33E−02 | 1.07 | 1,762 | 36,375 |
| rs401681 | T | 5 | 1,375,087 | 7.46E−04 | −0.06359 | 4,502 | 0.455 | 5.33E−02 | 0.94 | 1,762 | 36,375 |
| rs9364554 | C | 6 | 160,753,654 | 2.67E−01 | −0.02253 | 4,504 | 0.694 | 8.84E−02 | 0.94 | 1,761 | 36,376 |
| rs9364554 | T | 6 | 160,753,654 | 2.67E−01 | 0.022532 | 4,504 | 0.306 | 8.84E−02 | 1.07 | 1,761 | 36,376 |
| rs12155172 | A | 7 | 20,961,016 | 4.86E−02 | 0.042607 | 4,501 | 0.255 | 5.89E−01 | 1.02 | 1,762 | 36,360 |
| rs12155172 | G | 7 | 20,961,016 | 4.86E−02 | −0.04261 | 4,501 | 0.745 | 5.89E−01 | 0.98 | 1,762 | 36,360 |
| rs10486567 | A | 7 | 27,943,088 | 1.81E−01 | −0.02948 | 4,505 | 0.235 | 4.88E−03 | 0.89 | 1,762 | 36,379 |
| rs10486567 | G | 7 | 27,943,088 | 1.81E−01 | 0.029482 | 4,505 | 0.765 | 4.88E−03 | 1.12 | 1,762 | 36,379 |
| rs6465657 | C | 7 | 97,654,263 | 6.91E−01 | −0.00752 | 4,503 | 0.423 | 2.40E−01 | 1.04 | 1,762 | 36,319 |
| rs6465657 | T | 7 | 97,654,263 | 6.91E−01 | 0.007524 | 4,503 | 0.577 | 2.40E−01 | 0.96 | 1,762 | 36,319 |
| rs2928679 | A | 8 | 23,494,920 | 2.04E−01 | 0.023671 | 4,503 | 0.464 | 6.81E−02 | 1.06 | 1,761 | 36,364 |
| rs2928679 | G | 8 | 23,494,920 | 2.04E−01 | −0.02367 | 4,503 | 0.536 | 6.81E−02 | 0.94 | 1,761 | 36,364 |
| rs1512268 | C | 8 | 23,582,408 | 1.02E−05 | −0.08698 | 4,506 | 0.66 | 1.99E−03 | 0.9 | 1,763 | 36,400 |
| rs1512268 | T | 8 | 23,582,408 | 1.02E−05 | 0.08698 | 4,506 | 0.34 | 1.99E−03 | 1.12 | 1,763 | 36,400 |
| rs12543663 | A | 8 | 127,993,841 | 5.50E−01 | 0.012596 | 4,506 | 0.696 | 8.19E−04 | 0.88 | 1,763 | 36,400 |
| rs12543663 | C | 8 | 127,993,841 | 5.50E−01 | −0.0126 | 4,506 | 0.304 | 8.19E−04 | 1.14 | 1,763 | 36,400 |
| rs13252298 | A | 8 | 128,164,338 | 3.50E−01 | 0.019375 | 4,506 | 0.704 | 5.32E−05 | 1.17 | 1,763 | 36,400 |
| rs13252298 | G | 8 | 128,164,338 | 3.50E−01 | −0.01938 | 4,506 | 0.296 | 5.32E−05 | 0.85 | 1,763 | 36,400 |
| rs16901979 | A | 8 | 128,194,098 | 8.11E−04 | 0.18569 | 4,506 | 0.032 | 3.54E−17 | 1.92 | 1,763 | 36,400 |
| rs16901979 | C | 8 | 128,194,098 | 8.11E−04 | −0.18569 | 4,506 | 0.968 | 3.54E−17 | 0.52 | 1,763 | 36,400 |
| rs445114 | C | 8 | 128,392,363 | 1.27E−02 | −0.04946 | 4,503 | 0.327 | 2.08E−06 | 0.84 | 1,761 | 36,366 |
| rs445114 | T | 8 | 128,392,363 | 1.27E−02 | 0.049464 | 4,503 | 0.673 | 2.08E−06 | 1.2 | 1,761 | 36,366 |
| rs6983267 | G | 8 | 128,482,487 | 8.32E−02 | 0.032849 | 4,492 | 0.542 | 9.40E−04 | 1.12 | 1,759 | 36,219 |
| rs6983267 | T | 8 | 128,482,487 | 8.32E−02 | −0.03285 | 4,492 | 0.458 | 9.40E−04 | 0.89 | 1,759 | 36,219 |
| rs1447295 | A | 8 | 128,554,220 | 9.74E−03 | 0.078536 | 4,504 | 0.105 | 1.33E−20 | 1.57 | 1,762 | 36,389 |
| rs1447295 | C | 8 | 128,554,220 | 9.74E−03 | −0.07854 | 4,504 | 0.895 | 1.33E−20 | 0.64 | 1,762 | 36,389 |
| rs1571801 | G | 9 | 123,467,194 | 4.72E−02 | −0.04147 | 4,489 | 0.724 | 7.26E−02 | 1.07 | 1,758 | 36,234 |
| rs1571801 | T | 9 | 123,467,194 | 4.72E−02 | 0.041468 | 4,489 | 0.276 | 7.26E−02 | 0.93 | 1,758 | 36,234 |
| rs7920517 | A | 10 | 51,202,627 | 3.21E−04 | −0.06796 | 4,506 | 0.575 | 1.16E−03 | 0.89 | 1,763 | 36,400 |
| rs7920517 | G | 10 | 51,202,627 | 3.21E−04 | 0.067959 | 4,506 | 0.425 | 1.16E−03 | 1.12 | 1,763 | 36,400 |
| rs10993994 | C | 10 | 51,219,502 | 8.66E−06 | −0.0854 | 4,505 | 0.617 | 2.07E−03 | 0.9 | 1,763 | 36,384 |
| rs10993994 | T | 10 | 51,219,502 | 8.66E−06 | 0.085404 | 4,505 | 0.383 | 2.07E−03 | 1.11 | 1,763 | 36,384 |
| rs4962416 | C | 10 | 126,686,862 | 5.99E−01 | 0.011722 | 4,506 | 0.227 | 8.97E−01 | 1.01 | 1,763 | 36,400 |
| rs4962416 | T | 10 | 126,686,862 | 5.99E−01 | −0.01172 | 4,506 | 0.773 | 8.97E−01 | 0.99 | 1,763 | 36,400 |
| rs7127900 | A | 11 | 2,190,150 | 2.76E−01 | 0.027159 | 4,506 | 0.175 | 2.22E−03 | 1.15 | 1,763 | 36,400 |
| rs7127900 | G | 11 | 2,190,150 | 2.76E−01 | −0.02716 | 4,506 | 0.825 | 2.22E−03 | 0.87 | 1,763 | 36,400 |
| rs12418451 | A | 11 | 68,691,995 | 1.64E−01 | 0.029052 | 4,506 | 0.289 | 6.68E−05 | 1.16 | 1,763 | 36,400 |
| rs12418451 | G | 11 | 68,691,995 | 1.64E−01 | −0.02905 | 4,506 | 0.711 | 6.68E−05 | 0.86 | 1,763 | 36,400 |
| rs11228565 | A | 11 | 68,735,156 | 1.01E−02 | 0.081594 | 4,506 | 0.13 | 4.38E−05 | 1.25 | 1,763 | 36,400 |
| rs11228565 | G | 11 | 68,735,156 | 1.01E−02 | −0.08159 | 4,506 | 0.87 | 4.38E−05 | 0.8 | 1,763 | 36,400 |
| rs10896449 | A | 11 | 68,751,243 | 5.51E−01 | −0.01151 | 4,506 | 0.543 | 1.92E−04 | 0.88 | 1,763 | 36,400 |
| rs10896449 | G | 11 | 68,751,243 | 5.51E−01 | 0.011507 | 4,506 | 0.457 | 1.92E−04 | 1.14 | 1,763 | 36,400 |
| rs10896450 | A | 11 | 68,764,690 | 5.30E−01 | −0.01188 | 4,505 | 0.536 | 2.55E−04 | 0.88 | 1,762 | 36,381 |
| rs10896450 | G | 11 | 68,764,690 | 5.30E−01 | 0.011884 | 4,505 | 0.464 | 2.55E−04 | 1.13 | 1,762 | 36,381 |
| rs902774 | A | 12 | 51,560,171 | 2.20E−01 | 0.029519 | 4,506 | 0.193 | 3.95E−01 | 1.04 | 1,763 | 36,386 |
| rs902774 | G | 12 | 51,560,171 | 2.20E−01 | −0.02952 | 4,506 | 0.807 | 3.95E−01 | 0.96 | 1,763 | 36,386 |
| rs10778826 | A | 12 | 80,626,985 | 1.23E−01 | 0.029397 | 4,500 | 0.427 | 6.78E−02 | 0.94 | 1,762 | 36,363 |
| rs10778826 | G | 12 | 80,626,985 | 1.23E−01 | −0.0294 | 4,500 | 0.573 | 6.78E−02 | 1.07 | 1,762 | 36,363 |
| rs11861609 | C | 16 | 81,942,167 | 4.40E−01 | −0.01551 | 4,506 | 0.625 | 1.58E−01 | 0.95 | 1,763 | 36,400 |
| rs11861609 | G | 16 | 81,942,167 | 4.40E−01 | 0.015513 | 4,506 | 0.375 | 1.58E−01 | 1.05 | 1,763 | 36,400 |
| rs4782780 | C | 16 | 81,960,548 | 2.82E−01 | 0.021353 | 4,506 | 0.383 | 1.53E−01 | 1.05 | 1,763 | 36,400 |
| rs4782780 | T | 16 | 81,960,548 | 2.82E−01 | −0.02135 | 4,506 | 0.617 | 1.53E−01 | 0.95 | 1,763 | 36,400 |
| rs4054823 | C | 17 | 13,565,749 | 4.60E−01 | −0.01574 | 4,506 | 0.448 | 3.18E−02 | 0.92 | 1,763 | 36,400 |
| rs4054823 | T | 17 | 13,565,749 | 4.60E−01 | 0.015739 | 4,506 | 0.552 | 3.18E−02 | 1.09 | 1,763 | 36,400 |
| rs11649743 | A | 17 | 33,149,092 | 7.95E−01 | −0.00682 | 4,506 | 0.22 | 5.20E−02 | 0.91 | 1,763 | 36,400 |
| rs11649743 | G | 17 | 33,149,092 | 7.95E−01 | 0.006823 | 4,506 | 0.78 | 5.20E−02 | 1.1 | 1,763 | 36,400 |
| rs4430796 | A | 17 | 33,172,153 | 3.85E−09 | 0.116905 | 4,506 | 0.525 | 3.17E−05 | 1.17 | 1,763 | 36,400 |
| rs4430796 | G | 17 | 33,172,153 | 3.85E−09 | −0.11691 | 4,506 | 0.475 | 3.17E−05 | 0.86 | 1,763 | 36,400 |
| rs1859962 | G | 17 | 66,620,348 | 6.81E−01 | 0.007882 | 4,506 | 0.451 | 2.01E−04 | 1.14 | 1,763 | 36,400 |
| rs1859962 | T | 17 | 66,620,348 | 6.81E−01 | −0.00788 | 4,506 | 0.549 | 2.01E−04 | 0.88 | 1,763 | 36,400 |
| rs8102476 | C | 19 | 43,427,453 | 5.27E−02 | 0.03643 | 4,495 | 0.488 | 8.72E−04 | 1.12 | 1,754 | 36,238 |
| rs8102476 | T | 19 | 43,427,453 | 5.27E−02 | −0.03643 | 4,495 | 0.512 | 8.72E−04 | 0.89 | 1,754 | 36,238 |
| rs887391 | C | 19 | 46,677,464 | 3.77E−01 | −0.02005 | 4,504 | 0.219 | 8.30E−01 | 0.99 | 1,762 | 36,320 |
| rs887391 | T | 19 | 46,677,464 | 3.77E−01 | 0.020054 | 4,504 | 0.781 | 8.30E−01 | 1.01 | 1,762 | 36,320 |
| rs2659056 | C | 19 | 56,027,755 | 6.98E−04 | 0.085854 | 4,506 | 0.344 | 2.16E−01 | 1.06 | 1,763 | 36,400 |
| rs2659056 | T | 19 | 56,027,755 | 6.98E−04 | −0.08585 | 4,506 | 0.656 | 2.16E−01 | 0.94 | 1,763 | 36,400 |
| rs266849 | A | 19 | 56,040,902 | 6.32E−10 | 0.155396 | 4,496 | 0.834 | 3.66E−02 | 1.1 | 1,761 | 36,282 |
| rs266849 | G | 19 | 56,040,902 | 6.32E−10 | −0.1554 | 4,496 | 0.166 | 3.66E−02 | 0.91 | 1,761 | 36,282 |
| rs2735839 | A | 19 | 56,056,435 | 5.39E−17 | −0.22886 | 4,504 | 0.136 | 6.60E−03 | 0.87 | 1,763 | 36,364 |
| rs2735839 | G | 19 | 56,056,435 | 5.39E−17 | 0.22886 | 4,504 | 0.864 | 6.60E−03 | 1.15 | 1,763 | 36,364 |
| rs9623117 | C | 22 | 38,782,065 | 5.24E−01 | 0.014766 | 4,502 | 0.204 | 9.46E−01 | 1 | 1,762 | 36,381 |
| rs9623117 | T | 22 | 38,782,065 | 5.24E−01 | −0.01477 | 4,502 | 0.796 | 9.46E−01 | 1 | 1,762 | 36,381 |
| rs5759167 | G | 22 | 41,830,156 | 2.57E−01 | −0.02523 | 4,506 | 0.514 | 1.96E−02 | 1.1 | 1,763 | 36,400 |
| rs5759167 | T | 22 | 41,830,156 | 2.57E−01 | 0.02523 | 4,506 | 0.486 | 1.96E−02 | 0.91 | 1,763 | 36,400 |

Shown are association results for 47 SNPs reported to be associated with prostate cancer by various GWAS.
Our selection of SNPs is based on the NIH Catalog of Published Genome-Wide Association Studies; http://genome.gov/26525384#1.
Shown are association results for PSA levels;
two-sided P-values, the association effect in standardized units (s.u.) (see Methods), number (n) of individuals with PSA level measurements, and the allele frequency (freq.).
Shown are association results for prostate cancer in Iceland, the two-sided P-value, the odds ratio (OR) and the number (n) of patients with prostate cancer

TABLE 10
Association of the PSA variants with having undergone a biopsy of the prostate among Icelandic men

| | | | | | | | | Individuals | Individuals | |
|---|---|---|---|---|---|---|---|---|---|---|
| | | | | | | | | with | not with | |
| | | | | | | Individuals | Individuals not | biopsy, | biopsy, | |
| SNP | Allele | Chr | Position (bp) | P-value | OR | with biopsy (n) | with biopsy (n) | allele freq. | allele freq. | Comment |
| rs2736098 | A | 5 | 1,347,086 | 8.50E−03 | 1.11 | 2,216 | 41,323 | 0.35 | 0.34 | $ |
| rs401681 | C | 5 | 1,375,087 | 2.40E−03 | 1.09 | 2,513 | 41,509 | 0.57 | 0.55 | # |
| rs10993994 | T | 10 | 51,219,502 | 4.50E−02 | 1.06 | 2,342 | 39,737 | 0.4 | 0.39 | # |
| rs10788160 | A | 10 | 123,023,539 | 2.50E−02 | 1.08 | 2,302 | 37,835 | 0.33 | 0.31 | # |
| rs11067228 | A | 12 | 113,578,643 | 2.50E−01 | 1.04 | 2,347 | 39,340 | 0.57 | 0.56 | # |
| rs4430796 | A | 17 | 33,172,153 | 1.20E−04 | 1.13 | 2,338 | 39,621 | 0.55 | 0.53 | $ |
| rs17632542 | T | 19 | 56,053,569 | 4.20E−09 | 1.46 | 2,325 | 38,265 | 0.94 | 0.91 | $ |
| rs2735839 | G | 19 | 56,056,435 | 3.50E−05 | 1.21 | 2,368 | 39,551 | 0.89 | 0.86 | # |

Shown are: the allele associated with increased PSA levels, the number of individuals (n) that have undergone a biopsy of the prostate, the number of individuals (controls) not known to have undergone a biopsy of the prostate, the allele frequency (freq.) in each group of individuals, the odds ratio (OR), and the two-sided P-value.
# For those SNPs, the average number of persons with in-silico derived genotypes is 332, the remaining individuals were directly genotyped using the Illumina chip or single track SNP assays.
$ For those SNPs, 1,484 persons with biopsy and 36,369 persons not known to have a biopsy had their genotypes imputed based on the 2.5 million HapMap SNP data set or were genotyped using a single track SNP assays. The analysis are done separately for the different genotyping methods and the results combined using the Mantel-Haenszel model

TABLE 11
Association of the PSA variants with having a negative prostate biopsy outcome among Icelandic men
a. Results for SNPs and individuals genotyped with Illumina SNP chip

| | |
|---|---|
| Men | Frequency |

| | | | | | | with | | Men | |
|---|---|---|---|---|---|---|---|---|---|
| | | | | | | negative | | with | |
| | | | | | | biopsy | Controls | negative | |
| SNP | Allele | Chr | Position (bp) | P-value | OR | (n) | (n) | biopsy | Controls |
| rs10788160 | A | 10 | 123,023,539 | 4.20E−04 | 1.17 | 1,133 | 37,835 | 0.34 | 0.31 |
| rs10993994 | T | 10 | 51,219,502 | 0.48 | 1.03 | 1,143 | 39,737 | 0.39 | 0.39 |
| rs11067228 | A | 12 | 113,578,643 | 5.80E−03 | 1.12 | 1,151 | 39,340 | 0.59 | 0.56 |
| rs2735839 | G | 19 | 56,056,435 | 6.70E−06 | 1.35 | 1,137 | 39,551 | 0.9 | 0.86 |
| rs401681 | C | 5 | 1,375,087 | 0.037 | 1.09 | 1,169 | 41,509 | 0.57 | 0.55 |

b. Results for SNPs and individuals either imputed or genotyped using a Centaurus single track assay

| | | | | | | Imputed genotypes | | | | Single track assay genotypes | | | |
|---|---|---|---|---|---|---|---|---|---|---|---|---|---|
| | | | | | | Men | | Frequency | | Men | | Frequency | |
| | | | | | | with | | Men | | with | | Men | |
| | | | | | | negative | | with | | negative | | with | |
| | | | | | | biopsy | Controls | negative | | biopsy | Controls | negative | |
| SNP | Allele | Chr | Position (bp) | P-value | OR | (n) | (n) | biopsy | Controls | (n) | (n) | biopsy | Controls |
| rs2736098 | A | 5 | 1,347,086 | 0.025 | 1.13 | 488 | 36,369 | 0.36 | 0.35 | 492 | 4,954 | 0.32 | 0.28 |
| rs4430796 | A | 17 | 33,172,153 | 9.00E−03 | 1.14 | 488 | 36,369 | 0.56 | 0.53 | 491 | 3,252 | 0.54 | 0.51 |
| rs17632542 | T | 19 | 56,053,569 | 6.10E−09 | 1.82 | 488 | 36,369 | 0.94 | 0.91 | 480 | 1,896 | 0.96 | 0.91 |

Association results in Iceland for PSA SNPs in men that have had a prostate biopsy but have not been diagnosed with prostate cancer (a negative biopsy) compared with Icelandic controls that have not undergone a biopsy and are not known to have prostate cancer. Shown are: the allele associated with increased PSA levels, the number (n) of individuals that have undergone a biopsy of the prostate but were not diagnosed with prostate cancer (a negative biopsy), the number (n) of controls not known to have undergone a biopsy of the prostate and not known to have been diagnosed with prostate cancer, the allele frequency in each of groups, the odds ratio (OR), and the two-sided P-value. In the upper part of the table are results for individuals that were genotyped using the Illumina genotyping SNP chip. In the lower part of the table are the combined results for individuals either genotyped using Centaurus single track SNP assay or individuals that had their genotypes imputed based on the 2.5 million HapMap SNP data set.

TABLE 12
Association results for PSA SNPs and outcome from a bioppsy of the prostate, combined results for Iceland and UK

| | Allele | | | Persons | Persons | Persons | Persons | | | |
|---|---|---|---|---|---|---|---|---|---|---|
| | increasing | | | with pos. | with pos. | with neg. | with neg. | | | |
| | PSA- | | | biopsy | biopsy, | biopsy | biopsy, | OR | | |
| SNP | levels | Chr | Position (bp) | (n) | freq. | (n) | freq. | 95% CI | P-value | Pₕₑₜ |
| rs2736098 | A | 5 | 1,347,086 | 1,718 | 0.34 | 1,907 | 0.32 | 1.04 (0.94, 1.16) | 0.47 | 0.082 |
| rs10993994 | T | 10 | 51,219,502 | 1,696 | 0.41 | 2,082 | 0.4 | 1.05 (0.96, 1.15) | 0.31 | 0.82 |
| rs10788160 | A | 10 | 123,023,539 | 1,679 | 0.28 | 2,084 | 0.32 | 0.79 (0.71, 0.87) | 5.40E−06 | 0.092 |
| rs11067228 | A | 12 | 113,578,643 | 1,706 | 0.55 | 2,106 | 0.59 | 0.87 (0.79, 0.95) | 0.0034 | 0.51 |
| rs4430796 | A | 17 | 33,172,153 | 1,858 | 0.55 | 1,919 | 0.53 | 1.03 (0.97, 1.10) | 0.37 | 0.067 |
| rs17632542 | T | 19 | 56,053,569 | 1,873 | 0.93 | 1,924 | 0.95 | 0.77 (0.63, 0.95) | 0.013 | 0.56 |
| rs2735839 | G | 19 | 56,056,435 | 1,743 | 0.88 | 2,091 | 0.89 | 0.85 (0.74, 0.98) | 0.026 | 0.44 |

Shown are the results from a combined analysis of the Icelandic and UK study groups, the number of individuals (n) that have undergone a biopsy of the prostate and have been diagnosed with cancer of the prostate (positive biopsy; maximum number of individuals with genotypes used in the analysis is 1,870, of those 1,354 are from Iceland and 516 from the UK), the number of individuals (n) that have undergone a biopsy of the prostate and have not been diagnosed with cancer of the prostate (negative biopsy; maximum number of individuals with genotypes used in the analysis is 2,124, of those 1,169 are from Iceland and 955 from the UK), the allele associated with increased PSA levels and the allelic frequency (freq.), the odds ratio (OR), and the two-sided P-value. The OR and P-values were estimated using the Mantel-Haenszel model.

### Example 2

In order to summarize the overall effect on PSA levels, we combined the effect of the PSA variants, assuming a multiplicative model, independently for the Icelandic and UK study populations. We chose to include in the analysis only the four sequence variants, located near TERT, FGFR2TBX3 and KLK3 (rs2736098, rs10788160, rs11067228, and rs17632542, respectively) that are primarily associated with PSA levels. The variants at the MSMB and HNF1B loci were not included, since we consider them to be associated primarily with prostate cancer. Based on results from Iceland for the top 5% of the genetic PSA level distribution, the measured PSA levels are estimated to be increased by 23% to 47% compared to the population average. Similarly, for the bottom 5% of genetic PSA level distribution, the measured PSA levels is estimated to be decreased by 30% to 56% compared to the population average. In the UK study population the estimated relative effect on PSA levels are even greater; the range of increase is 40% to 92% for the top 5% of the distribution with the greatest genotypic effect compared to the population average, whereas for the bottom 5% of the distribution, the range of decrease is 53% to 80% compared to the population average.

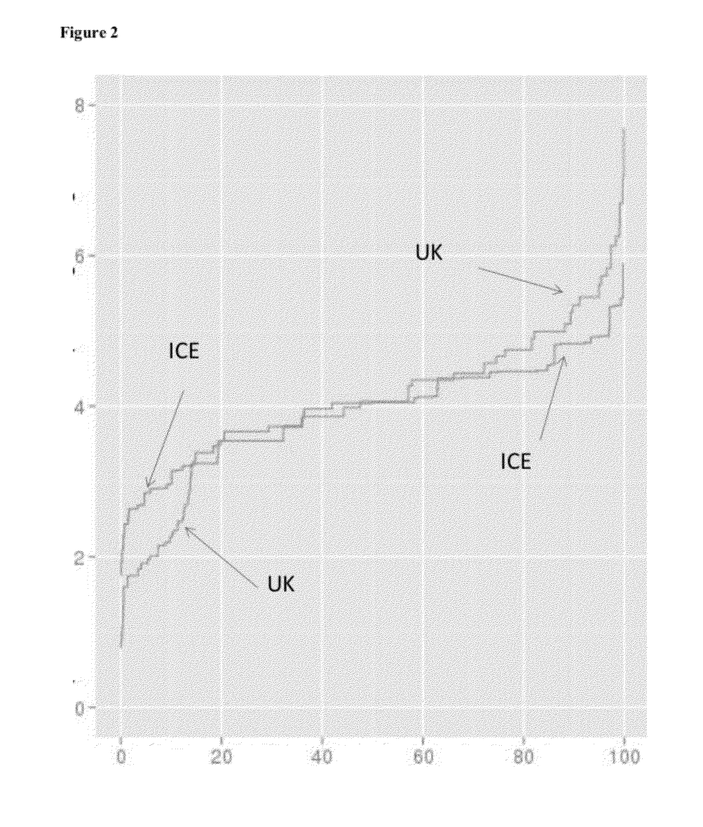

To apply the above to demonstrate how the genetic effect of the four PSA sequence variants influences individual PSA levels, we calculated a personalized PSA cutoff value corresponding to the commonly used cutoff of 4 ng/ml. This was done by multiplying the value of 4 ng/ml with the estimated relative genetic effect for the PSA SNPs. For individuals with the highest (top 5% of the distribution) genotypic effect, the personalized PSA cutoff value increased from 4 ng/ml to cutoff values between 4.9 and 5.9 ng/ml based on the estimates from Iceland, and to cutoff values between 5.6 and 7.7 ng/ml based on the UK estimates. For the bottom 5% of the genetic relative effect distribution, the personalized PSA cutoff values move from 4 ng/ml to cutoff values between 1.7 and 2.8 ng/ml according to the Icelandic estimates, and to cutoff values between 0.8 and 1.9 ng/ml according to the UK estimates (see FIG. 2). These data demonstrate that for a substantial fraction of men undergoing PSA-based prostate cancer screening, the personalized PSA cutoff value is shifted following correction for the effect of the PSA sequence variants. If applied clinically, men would be reclassified with respect to whether or not they should undergo a biopsy.

Our results from estimating the combined relative effect of the 4 variants primarily associated with PSA levels demonstrate a considerable variation in PSA levels between individuals based on their genotypes of these 4 variants. By applying the combined genetic effect on commonly used PSA cutoff values, a personalized PSA cutoff value can be obtained. Thus our data indicate that for a substantial fraction of men undergoing PSA-based prostate cancer screening, the personalized PSA cutoff value (for the decision of doing a biopsy or not) is shifted and hence men would be reclassified with respect to whether or not they should undergo a biopsy. This reclassification is likely to affect both the sensitivity and the specificity of the PSA test, and thereby, also the long term outcome of the patients since early diagnosis is the most powerful way to improve the patient's prognosis. For a screening test as important and widely used as the PSA test, having a better way to interpret the measured PSA level is likely to improve substantially the clinical performance of the test.

### Example 3

### Materials and Methods

### Study Subjects

Icelandic study population. Results from PSA testing were collected from the three clinical laboratories performing the great majority of all PSA measurements in Iceland. The series of data spanned a period of 15 years (from 1994 to 2009). In total we had information about PSA values from 15,757 individuals. The men have not been diagnosed with prostate cancer according to the nation-wide Icelandic Cancer Registry (ICR), and had not undergone TURP between 1983 and 2008, based on a list from the Landspitali-University Hospital where 90% of all TURP procedures in the country are performed.

Icelandic men diagnosed with prostate cancer were identified based on a nationwide list from the ICR that contained all 4,732 Icelandic prostate cancer patients diagnosed from Jan. 1, 1955, to Dec. 31, 2008. The Icelandic prostate cancer sample collection included 2,289 patients (diagnosed from December 1974 to December 2008) who were recruited from November 2000 until June 2009. A total of 2,249 patients were included in the study which all had genotypes from a genome wide SNP genotyping effort, using the Infinium II assay method and the Sentrix HumanHap300 BeadChip (Illumina, San Diego, Calif., USA) or a Centaurus single SNP genotyping assay (see Supplementary Materials). The mean age at diagnosis for the consenting patients is 70.7 years (ranging from 40 to 96 years), while the mean age at diagnosis is 73 years for all prostate cancer patients in the ICR. The median time from diagnosis to blood sampling is 2 years (range 0 to 26 years). In the present study, for all populations, aggressive prostate cancer is defined as: Gleason >7 and/or T3 or higher and/or node positive and/or metastatic disease, while the less aggressive disease is defined as Gleason <7 and T2 or lower. The Icelandic men diagnosed with benign hyperplasia of the prostate (BPH) were identified based on a list of men undergoing TURP between 1983 and 2008 at the Landspitali-National Hospital in Iceland.

The 35,470 controls (15,359 men (43.3%) and 20,111 femen (56.7%)) used in this study consisted of individuals recruited through different genetic research projects at deCODE. The individuals have been diagnosed with common diseases of the cardio-vascular system (e.g. stroke or myocardial infraction), psychiatric and neurological diseases (e.g. schizophrenia, bipolar disorder), endocrine and autoimmune system (e.g. type 2 diabetes, asthma), malignant diseases other than prostate cancer as well as individuals randomly selected from the Icelandic genealogical database. No single disease project represented more than 6% of the total number of controls. The controls had a mean age of 84 years and the range was from 8 to 105 years. The controls were absent from the nation-wide list of prostate cancer patients according to the ICR. The DNA for both the Icelandic cases and controls was isolated from whole blood using standard methods.

The study was approved by the Data Protection Commission of Iceland and the National Bioethics Committee of Iceland. Written informed consent was obtained from all patients and controls. Personal identifiers associated with medical information and blood samples were encrypted with a third-party encryption system as previously described (Gulcher, J. R., et al. Eur J. Hum Genet. 8:739-42 (2000)).

UK study population. In the ‘Prostate Testing for Cancer and Treatment’ trial (ProtecT), men aged 50-69 years were contacted and provided with information about the uncertainty surrounding PSA testing, detection and radical treatment of early prostate cancer, and offered an appointment for counseling and PSA testing. Recruitment took place at nine sites in the UK; 94,427 men agreed to be tested (50% of men contacted) and 8,807 (˜9%) had a raised PSA level. Of those with raised PSA levels, 2,022 (23%) were diagnosed with prostate cancer; 229 men (˜12%) had locally advanced (T3 or T4) or metastatic cancers, the rest having clinically localized (T1c or T2) disease. Men with a PSA level of ≧20 ng/mL were excluded from the trial. Those with locally confined cancers (mostly T1c, but some T2a and T2b) and with PSA levels of <20 ng/mL were offered randomization into a three-arm trial of treatment (random assignment between active monitoring, radical prostatectomy or radical radiotherapy). Participants will be followed up for ≧10 years. Study participants found to have locally advanced (≧T3) or distantly advanced disease were not eligible for the ProtecT treatment trial, and were referred for routine UK National Health Service care. Ethical approval for the ProtecT study was obtained from Trent Multi-Centre Research Ethics Committee.

From the ProtecT trial study group, the following number of samples were selected for the present study: 524 men with PSA values >3 ng/ml and diagnosed with prostate cancer after undergoing a needle biopsy (average age at diagnosis is 63.0 years), 960 men with PSA values between 3 ng/ml and 10 ng/ml but not diagnosed with prostate cancer after undergoing a needle biopsy (average age at PSA measurement is 62.4 years), and 454 men with PSA values <3 ng/ml (average age at PSA measurement is 62.7 years).

Dutch study population. The total number of Dutch prostate cancer cases used in this study was 1,100. The Dutch study population consisted of two recruitment-sets of prostate cancer cases; Group-A was comprised of 360 hospital-based cases recruited from January 1999 to June 2006 at the Urology Outpatient Clinic of the Radboud University Nijmegen Medical Centre (RUNMC); Group-B consisted of 707 cases recruited from June 2006 to December 2006 through a population-based cancer registry held by the Comprehensive Cancer Centre IKO. Both groups were of self-reported European descent. The average age at diagnosis for patients in Group-A was 63 years (median 63 years; range 43 to 83 years). The average age at diagnosis for patients in Group-B was 65 years (median 66 years; range 43 to 75 years). The 2,021 control individuals (1,004 men and 1,017 femen) were cancer free and were matched for age with the cases. They were recruited within a project entitled “The Nijmegen Biomedical Study”, in the Netherlands. This is a population-based survey conducted by the Department of Epidemiology and Biostatistics and the Department of Clinical Chemistry of RUNMC, in which 9,371 individuals participated from a total of 22,500 age and sex stratified, randomly selected inhabitants of Nijmegen. Control individuals from the Nijmegen Biomedical Study were invited to participate in a study on gene-environment interactions in multifactorial diseases, such as cancer. All the 2,021 participants in the present study are of self-reported European descent and were fully informed about the goals and the procedures of the study. The study protocol was approved by the Institutional Review Board of Radboud University and all study subjects gave written informed consent.

Spanish study population. The Spanish study population used in this study consisted of 618 prostate cancer cases. The cases were recruited from the Oncology Department of Zaragoza Hospital in Zaragoza, Spain, from June 2005 to September 2007. All patients were of self-reported European descent. Clinical information including age at onset, grade and stage was obtained from medical records. The average age at diagnosis for the patients was 69 years (median 70 years) and the range was from 44 to 83 years. The 1,605 Spanish control individuals (737 men and 868 femen) were approached at the University Hospital in Zaragoza, and the men were prostate cancer free at the time of recruitment. Study protocols were approved by the Institutional Review Board of Zaragoza University Hospital. All subjects gave written informed consent.

Chicago study population. The Chicago study population used consisted of 1,560 prostate cancer cases. The cases were recruited from the Pathology Core of Northwestern University's Prostate Cancer Specialized Program of Research Excellence (SPORE) from May 2002 to May 2009. The average age at diagnosis for the patients was 60 years (median 59 years) and the range was from 39 to 87 years. The 1,172 European American controls (781 men and 391 femen) were recruited as healthy control subjects for genetic studies at the University of Chicago and Northwestern University Medical School, Chicago, US. All individuals from Chicago included in this report were of self-reported European descent. Study protocols were approved by the Institutional Review Boards of Northwestern University and the University of Chicago. All subjects gave written informed consent.

Romanian study population. The Romanian study population used in this study consisted of 362 prostate cancer cases. The cases were recruited from the Urology Clinic

“Theodor Burghele” of The University of Medicine and Pharmacy “Carol Davila” Bucharest, Romania, from May 2008 to November 2009. All patients were of self-reported European descent. Clinical information including age at onset, grade and stage were obtained from medical records at the hospital. The average age at diagnosis for the cases was 70 years (median 71 years) and the range was from 46 to 89 years. The 182 Romanian controls were recruited at the General Surgery Clinic “St. Mary” and at the Urology Clinic “Theodor Burghele” of The University of Medicine and Pharmacy “Carol Davila” Bucharest, Romania. The average age for controls was 60 years (median 62 years) with a range from 19 to 87 years. The controls were cancer free at the time of recruitment. PSA values were tested for men. Study protocols were approved by the National Ethical Board of the Romanian Medical Doctors Association in Romania. All subjects gave written informed consent.

### Genotyping

As a part of ongoing research projects at deCODE, 38,541 Icelandic individuals have been successfully genotyped with either the Infinium HumanHap300 or the 370K SNP chip (Illumina, San Diego, Calif., USA), containing haplotype tagging SNPs derived from phase I of the International HapMap project. After quality control, 304,070 SNPs were available for the GWAS of PSA levels. Any samples with a call rate below 98% were excluded from the analysis. Single SNP genotyping of the PSA follow-up samples from Iceland and the UK and the prostate cancer case-control groups from The Netherlands, Spain, Romania, and Chicago was carried out by deCODE Genetics in Reykjavik, Iceland, applying the Centaurus (Nanogen) platform. The quality of each Centaurus SNP assay was evaluated by genotyping each assay in the CEU and/or YRI HapMap samples and comparing the results with the HapMap publicly released data. Assays with >1.5% mismatch rate were not used and a linkage disequilibrium (LD) test was used for markers known to be in LD.

### Association Testing of Quantitative Traits

### PSA Level

Two populations were used to study PSA levels; Iceland and UK. To study PSA levels among unaffected men in Iceland, we excluded subjects who had been diagnosed with prostate cancer as recorded by the ICR (between 1955 and 2008) or were known to have undergone TURP between 1983 and 2008. PSA levels were corrected for age at measurement for each center separately, using a generalized additive model with a smooth component on the age. Also, the PSA levels were standardized so that they had a normal distribution, using a quantile standardization. Most subjects had more than two PSA measurements. Hence, we used the mean of the adjusted and standardized PSA values for each individual.

For each SNP a classical linear regression using the genotype as an additive covariate and PSA as a response, was fitted to test for association. In addition to testing the standardized value, we also performed an analysis using log-transformed values which we then back-transformed to report the effect under a multiplicative model. We report significance levels based on the standardized values and the association effect based on both the standardized value and under the multiplicative model.

PSA measurements exist for many more Icelandic individuals than those who have been genotyped using an Illumina SNP chip. We used the available genotype information on the relatives of individuals who had not been genotyped in order to extract more information on association from our data (in-silico genotyping). In total we had access to PSA levels of 4,620 individuals genotyped on Illumina chips, all containing the 317K HumanHap SNP panel. The analysis was augmented with data from 9,218 Icelanders with PSA measurements whose genetic information could be partially inferred from genotyped relatives that belong to the set of the 38,541 chip typed Icelanders. This augmentation is equivalent to an additional 2,918 individuals. We have previously applied this method to the analysis of height and details can be found in a recent publication (Gudbjartsson, D. F. et al. Nat. Genet. 40:609-15 (2008)). After the initial scan, we followed-up the top markers, using 1,919 men genotyped with Centaurus single track assay. Our final analysis eventually included all genotype data, derived from: chip-, single-track-, and in-silico genotyping.

To study PSA levels in the UK samples, we used 454 men with a single PSA measurement with a value between 0 and 3 ng/ml from the ProtecT trial and directly genotyped with Centaurus single track assay. Measurements were standardized and adjusted for age at measurement and center.

To calculate a combined significance for Iceland and the UK, we performed a two degree of freedom test on the sum of the individual χ² values. To model the genotypic effect of SNPs on PSA level in each population, we use the estimated allelic effect based on the multiplicative model within each locus (see above) and assume Hardy-Weinberg equilibrium. When combining the effect of multiple SNPs, we assume linkage equilibrium between loci and use a multiplicative model. When performing a case only analysis among prostate cancer patients of the six populations to study the association between SNPs and age at diagnosis, we use a linear regression with age at diagnosis as response and the allele count as an additive covariate.

### Association Testing of Binary Traits

For case control association analysis, for example when comparing prostate cancer cases, benign prostatic hyperplasia cases or biopsied individuals to population controls and within group comparisons (aggressive vs. non-aggressive, biopsy pos. vs. biopsy neg.), we used a standard likelihood ratio statistic, implemented in the NEMO software to calculate two-sided P values for each individual allele, assuming a multiplicative model for risk (Gretarsdottir, S. et al. Nat Genet. 35:131-8 (2003)). Combined significance levels were calculated using a Mantel-Haenszel model. Heterogeneity was examined using a likelihood ratio test by comparing the null hypothesis of the effect being the same in all populations to the alternative hypothesis of each population having a different effect.

### Finemapping of the Six PSA Associated Loci

To investigate further the top six loci from the GWAS, we analyzed the association of imputed genotypes based on HapMap CEU for a window of 500 Kb centered on the most significant SNP at each loci. For the individuals directly genotyped on chip, SNP imputation was based on the Phase II CEU HapMap samples and was done using IMPUTE. Association testing was performed using a logistic regression with the allele count as a covariate. For a given locus, we performed multivariate analysis using genotypes from different SNPs as covariates and standardized and corrected PSA value as the response to adjust the association of one SNP for the other SNP.

### Example 4

We investigated the observed correlation of surrogate markers with PSA levels. For this purpose, genotypes for surrogates of the markers rs401681, rs2736098, rs10788160, rs11067228, rs10993994, rs4430796, rs2735839 and rs17632542 were imputed based on the 1000 genomes data set (available at 1000genomes.org). All the surrogates were selected using a cutoff of r²>0.2 (see Table 1).

Results are shown in Table 13. As can be seen, all the surrogate markers are significantly associated with PSA levels, showing that these markers can all be useful for assessing the effect of genetic variants on PSA levels.

TABLE 13
Association of surrogate markers with PSA levels.

| | | POS in | | MAF | # of | | | | Decrease | Increase | Seq ID |
|---|---|---|---|---|---|---|---|---|---|---|---|
| SNP | Chr | B36 | A1/A2 | (A1) | cases | Effect | P-value | info | Allele | Allele | NO: |
| s.51165690 | chr10 | 51165690 | C/A | 0.41 | 4276 | 0.09694 | 1.87E−06 | 1 | A | C | 468 |
| s.51172808 | chr10 | 51172808 | G/C | 0.46 | 4276 | 0.0868 | 2.58E−06 | 1 | C | G | 475 |
| s.51175013 | chr10 | 51175013 | A/G | 0.25 | 4276 | 0.09929 | 1.57E−04 | 0.93 | G | A | 483 |
| s.56037076 | chr19 | 56037076 | C/T | 0.12 | 4278 | 0.19928 | 1.40E−09 | 0.85 | C | T | 685 |
| s.56054527 | chr19 | 56054527 | G/T | 0.13 | 4278 | 0.25785 | 3.71E−19 | 0.94 | G | T | 694 |
| s.56058688 | chr19 | 56058688 | A/T | 0.03 | 4278 | 0.29527 | 3.15E−07 | 0.78 | A | T | 697 |
| s.56060000 | chr19 | 56060000 | C/A | 0.03 | 4278 | 0.29869 | 2.98E−07 | 0.78 | C | A | 699 |
| s.56066550 | chr19 | 56066550 | A/T | 0.03 | 4278 | 0.30362 | 2.63E−07 | 0.77 | A | T | 702 |
| s.56066560 | chr19 | 56066560 | G/C | 0.03 | 4278 | 0.30363 | 2.63E−07 | 0.77 | G | C | 703 |
| s.56066619 | chr19 | 56066619 | T/G | 0.03 | 4278 | 0.30374 | 2.62E−07 | 0.77 | T | G | 704 |
| rs1058205 | chr19 | 56055210 | C/T | 0.18 | 4286 | 0.2032 | 2.84E−17 | 1 | C | T | 12 |
| rs1061657 | chr12 | 113592519 | C/T | 0.23 | 4277 | 0.08141 | 8.87E−04 | 0.96 | C | T | 13 |
| rs10749412 | chr10 | 123007551 | T/A | 0.41 | 4280 | 0.06583 | 3.70E−04 | 1 | A | T | 17 |
| rs10749413 | chr10 | 123015655 | T/A | 0.38 | 4280 | 0.08499 | 1.77E−05 | 1 | A | T | 18 |
| rs10763534 | chr10 | 51204926 | C/T | 0.43 | 4276 | 0.07645 | 4.92E−05 | 1 | T | C | 19 |
| rs10763536 | chr10 | 51205807 | G/A | 0.45 | 4276 | 0.07439 | 8.99E−05 | 1 | A | G | 20 |
| rs10763546 | chr10 | 51206405 | C/G | 0.43 | 4276 | 0.07784 | 3.65E−05 | 1 | G | C | 21 |
| rs10763576 | chr10 | 51208819 | A/T | 0.43 | 4276 | 0.07793 | 3.76E−05 | 1 | T | A | 22 |
| rs10763588 | chr10 | 51209768 | G/T | 0.43 | 4276 | 0.07814 | 3.60E−05 | 1 | T | G | 23 |
| rs10788154 | chr10 | 123011231 | C/A | 0.41 | 4280 | 0.06866 | 2.14E−04 | 1 | A | C | 25 |
| rs10788159 | chr10 | 123020775 | G/A | 0.29 | 4280 | 0.09245 | 1.85E−05 | 0.99 | A | G | 26 |
| rs10788162 | chr10 | 123027299 | G/A | 0.4 | 4280 | 0.08664 | 7.46E−06 | 1 | A | G | 27 |
| rs10788163 | chr10 | 123029792 | G/T | 0.28 | 4280 | 0.09687 | 4.98E−06 | 0.99 | T | G | 28 |
| rs10788164 | chr10 | 123032835 | T/C | 0.37 | 4280 | 0.08831 | 5.86E−06 | 1 | C | T | 29 |
| rs10788165 | chr10 | 123034204 | G/T | 0.37 | 4280 | 0.08936 | 4.42E−06 | 1 | T | G | 30 |
| rs10788166 | chr10 | 123036532 | G/A | 0.28 | 4280 | 0.09745 | 3.41E−06 | 1 | A | G | 31 |
| rs10788167 | chr10 | 123044008 | A/T | 0.28 | 4280 | 0.09678 | 4.07E−06 | 1 | T | A | 32 |
| rs10825652 | chr10 | 51180767 | A/G | 0.44 | 4276 | 0.08462 | 7.09E−06 | 1 | G | A | 33 |
| rs10826075 | chr10 | 51197376 | G/C | 0.3 | 4276 | 0.0852 | 2.30E−04 | 0.97 | C | G | 34 |
| rs10826125 | chr10 | 51200511 | G/A | 0.44 | 4276 | 0.07811 | 3.26E−05 | 1 | A | G | 35 |
| rs10826127 | chr10 | 51200763 | G/A | 0.43 | 4276 | 0.07836 | 2.56E−05 | 1 | A | G | 36 |
| rs10886880 | chr10 | 123003911 | C/T | 0.31 | 4280 | 0.07272 | 3.18E−04 | 1 | T | C | 37 |
| rs10886882 | chr10 | 123017023 | T/C | 0.36 | 4280 | 0.08932 | 9.07E−06 | 0.99 | C | T | 38 |
| rs10886883 | chr10 | 123017171 | G/C | 0.38 | 4280 | 0.08636 | 1.30E−05 | 1 | C | G | 39 |
| rs10886885 | chr10 | 123020471 | T/G | 0.29 | 4280 | 0.09362 | 1.51E−05 | 0.99 | G | T | 40 |
| rs10886886 | chr10 | 123020859 | G/T | 0.28 | 4280 | 0.09518 | 1.01E−05 | 0.99 | T | G | 41 |
| rs10886887 | chr10 | 123023168 | T/C | 0.3 | 4280 | 0.09331 | 8.21E−06 | 0.99 | C | T | 42 |
| rs10886890 | chr10 | 123027193 | G/A | 0.3 | 4280 | 0.09356 | 7.25E−06 | 0.99 | A | G | 43 |
| rs10886893 | chr10 | 123034442 | C/T | 0.28 | 4280 | 0.09729 | 3.63E−06 | 1 | T | C | 44 |
| rs10886894 | chr10 | 123036863 | C/T | 0.27 | 4280 | 0.09838 | 2.56E−06 | 1 | T | C | 45 |
| rs10886895 | chr10 | 123037303 | A/C | 0.28 | 4280 | 0.0961 | 4.53E−06 | 1 | C | A | 46 |
| rs10886896 | chr10 | 123037386 | A/C | 0.28 | 4280 | 0.09815 | 2.82E−06 | 1 | C | A | 47 |
| rs10886897 | chr10 | 123037630 | C/T | 0.28 | 4280 | 0.09702 | 3.63E−06 | 1 | T | C | 48 |
| rs10886898 | chr10 | 123037681 | G/T | 0.28 | 4280 | 0.09733 | 3.37E−06 | 1 | T | G | 49 |
| rs10886899 | chr10 | 123037711 | T/G | 0.27 | 4280 | 0.09743 | 3.10E−06 | 1 | G | T | 50 |
| rs10886900 | chr10 | 123037998 | G/A | 0.28 | 4280 | 0.097 | 3.61E−06 | 1 | A | G | 51 |
| rs10886901 | chr10 | 123038120 | C/T | 0.28 | 4280 | 0.09662 | 3.93E−06 | 1 | T | C | 52 |
| rs10886902 | chr10 | 123039254 | C/T | 0.28 | 4280 | 0.09804 | 2.74E−06 | 1 | T | C | 53 |
| rs10886903 | chr10 | 123039425 | G/C | 0.27 | 4280 | 0.09682 | 3.43E−06 | 1 | C | G | 54 |
| rs10908278 | chr17 | 33174065 | T/A | 0.46 | 4273 | 0.10932 | 1.32E−08 | 1 | T | A | 57 |
| rs11004246 | chr10 | 51165355 | C/T | 0.4 | 4276 | 0.09922 | 1.13E−06 | 1 | T | C | 58 |
| rs11004324 | chr10 | 51166629 | G/T | 0.4 | 4276 | 0.09888 | 1.10E−06 | 1 | T | G | 59 |
| rs11004409 | chr10 | 51168025 | C/G | 0.46 | 4276 | 0.08842 | 1.75E−06 | 1 | G | C | 60 |
| rs11004415 | chr10 | 51168187 | A/G | 0.46 | 4276 | 0.08708 | 2.48E−06 | 1 | G | A | 61 |
| rs11004422 | chr10 | 51168342 | G/A | 0.46 | 4276 | 0.08713 | 2.43E−06 | 1 | A | G | 62 |
| rs11004435 | chr10 | 51168499 | A/C | 0.46 | 4276 | 0.08827 | 1.77E−06 | 1 | C | A | 63 |
| rs11006207 | chr10 | 51208182 | T/C | 0.43 | 4276 | 0.07769 | 3.96E−05 | 1 | C | T | 64 |
| rs11006274 | chr10 | 51210297 | T/C | 0.43 | 4276 | 0.07774 | 3.95E−05 | 1 | C | T | 65 |
| rs11199862 | chr10 | 123012946 | A/G | 0.31 | 4280 | 0.07563 | 1.96E−04 | 1 | G | A | 67 |
| rs11199866 | chr10 | 123015727 | A/G | 0.38 | 4280 | 0.08587 | 1.44E−05 | 1 | G | A | 68 |
| rs11199867 | chr10 | 123017394 | T/G | 0.38 | 4280 | 0.08549 | 1.62E−05 | 1 | G | T | 69 |
| rs11199868 | chr10 | 123018329 | A/T | 0.28 | 4280 | 0.09452 | 1.15E−05 | 0.99 | T | A | 70 |
| rs11199869 | chr10 | 123020055 | G/A | 0.28 | 4280 | 0.0963 | 7.73E−06 | 0.99 | A | G | 71 |
| rs11199871 | chr10 | 123020940 | A/C | 0.29 | 4280 | 0.09217 | 1.91E−05 | 0.99 | C | A | 72 |
| rs11199872 | chr10 | 123021180 | A/G | 0.28 | 4280 | 0.09551 | 9.38E−06 | 0.99 | G | A | 73 |
| rs11199874 | chr10 | 123022509 | A/G | 0.3 | 4280 | 0.09269 | 9.45E−06 | 0.99 | G | A | 74 |
| rs11199879 | chr10 | 123035202 | C/T | 0.27 | 4280 | 0.09777 | 3.16E−06 | 1 | T | C | 75 |
| rs11199881 | chr10 | 123035860 | C/T | 0.28 | 4280 | 0.09625 | 4.32E−06 | 1 | T | C | 76 |
| rs1125527 | chr10 | 123009606 | A/G | 0.41 | 4280 | 0.06622 | 3.41E−04 | 1 | G | A | 85 |
| rs1125528 | chr10 | 123009942 | A/T | 0.31 | 4280 | 0.07425 | 2.44E−04 | 1 | T | A | 86 |
| rs11263761 | chr17 | 33171888 | G/A | 0.48 | 4273 | 0.1151 | 6.75E−09 | 1 | G | A | 87 |
| rs11263763 | chr17 | 33177678 | G/A | 0.46 | 4273 | 0.11044 | 1.43E−08 | 1 | G | A | 88 |
| rs11593361 | chr10 | 51209162 | A/G | 0.45 | 4276 | 0.08239 | 2.30E−05 | 1 | G | A | 90 |
| rs11598592 | chr10 | 123033379 | A/G | 0.41 | 4280 | 0.08197 | 2.99E−05 | 1 | G | A | 91 |
| rs11599333 | chr10 | 51169661 | C/A | 0.46 | 4276 | 0.08748 | 2.16E−06 | 1 | A | C | 92 |
| rs11609105 | chr12 | 113586865 | C/A | 0.22 | 4277 | 0.08359 | 8.74E−04 | 0.95 | C | A | 93 |
| rs11651052 | chr17 | 33176494 | A/G | 0.46 | 4273 | 0.11122 | 8.19E−09 | 1 | A | G | 94 |
| rs11651755 | chr17 | 33173953 | C/T | 0.46 | 4273 | 0.10989 | 1.11E−08 | 1 | C | T | 95 |
| rs11657964 | chr17 | 33174880 | A/G | 0.42 | 4273 | 0.09417 | 4.44E−07 | 1 | A | G | 96 |
| rs11658063 | chr17 | 33177985 | C/G | 0.41 | 4273 | 0.09747 | 2.76E−07 | 1 | C | G | 97 |
| rs12146156 | chr10 | 123014406 | C/T | 0.29 | 4280 | 0.0939 | 1.42E−05 | 0.99 | T | C | 99 |
| rs12146366 | chr10 | 123014670 | T/C | 0.29 | 4280 | 0.09314 | 1.66E−05 | 0.99 | C | T | 100 |
| rs12413088 | chr10 | 123042718 | T/C | 0.27 | 4286 | 0.09741 | 2.96E−06 | 1 | C | T | 102 |
| rs12413648 | chr10 | 123028887 | A/G | 0.27 | 4280 | 0.09755 | 4.03E−06 | 0.99 | G | A | 103 |
| rs12415826 | chr10 | 123036368 | C/T | 0.28 | 4280 | 0.09745 | 3.43E−06 | 1 | T | C | 104 |
| rs12761612 | chr10 | 123021400 | A/G | 0.28 | 4280 | 0.09499 | 1.05E−05 | 0.99 | G | A | 106 |
| rs12763717 | chr10 | 51170880 | G/C | 0.46 | 4276 | 0.08739 | 2.21E−06 | 1 | C | G | 107 |
| rs12781411 | chr10 | 51161595 | T/C | 0.4 | 4276 | 0.1019 | 9.22E−07 | 0.99 | C | T | 109 |
| rs174776 | chr19 | 56051664 | T/C | 0.13 | 4278 | 0.20027 | 3.48E−12 | 0.94 | T | C | 113 |
| rs17632542 | chr19 | 56053569 | C/T | 0.12 | 4278 | 0.27439 | 4.18E−18 | 0.88 | C | T | 114 |
| rs1873450 | chr10 | 122996264 | G/T | 0.31 | 4276 | 0.07132 | 4.02E−04 | 1 | T | G | 116 |
| rs1873451 | chr10 | 123000467 | C/T | 0.41 | 4280 | 0.06542 | 3.93E−04 | 1 | T | C | 117 |
| rs1873452 | chr10 | 123000564 | C/T | 0.41 | 4280 | 0.06638 | 3.21E−04 | 1 | T | C | 118 |
| rs2005705 | chr17 | 33170413 | A/G | 0.46 | 4273 | 0.11431 | 5.16E−09 | 1 | A | G | 128 |
| rs2125770 | chr10 | 51184830 | T/C | 0.46 | 4276 | 0.08553 | 3.12E−06 | 1 | C | T | 129 |
| rs2201026 | chr10 | 122998993 | G/T | 0.45 | 4276 | 0.06221 | 1.04E−03 | 1 | T | G | 132 |
| rs2249986 | chr10 | 51191690 | T/G | 0.43 | 4276 | 0.08158 | 1.35E−05 | 1 | G | T | 133 |
| rs2569735 | chr19 | 56056081 | A/G | 0.14 | 4278 | 0.22381 | 4.26E−17 | 1 | A | G | 137 |
| rs2611489 | chr10 | 51194895 | G/A | 0.43 | 4276 | 0.07625 | 4.00E−05 | 1 | A | G | 138 |
| rs2611506 | chr10 | 51188793 | C/T | 0.43 | 4276 | 0.07949 | 1.96E−05 | 1 | T | C | 139 |
| rs2611507 | chr10 | 51188679 | T/C | 0.43 | 4276 | 0.08293 | 1.00E−05 | 1 | C | T | 140 |
| rs2611508 | chr10 | 51188053 | T/A | 0.43 | 4276 | 0.08156 | 1.18E−05 | 1 | A | T | 141 |
| rs2611509 | chr10 | 51186258 | G/A | 0.44 | 4276 | 0.08275 | 1.03E−05 | 1 | A | G | 142 |
| rs2611512 | chr10 | 51185540 | A/G | 0.46 | 4282 | 0.08499 | 3.66E−06 | 1 | G | A | 143 |
| rs2611513 | chr10 | 51185463 | C/T | 0.44 | 4276 | 0.08306 | 9.58E−06 | 1 | T | C | 144 |
| rs2659051 | chr19 | 56037380 | C/G | 0.15 | 4278 | 0.17727 | 4.32E−10 | 0.92 | C | G | 145 |
| rs2659122 | chr19 | 56054838 | C/T | 0.26 | 4278 | 0.12281 | 1.56E−08 | 0.99 | C | T | 146 |
| rs2659124 | chr19 | 56046409 | A/T | 0.13 | 4278 | 0.19749 | 7.45E−12 | 0.94 | A | T | 147 |
| rs266849 | chr19 | 56040902 | G/A | 0.17 | 4287 | 0.14737 | 1.99E−09 | 1 | G | A | 148 |
| rs266878 | chr19 | 56050926 | G/C | 0.13 | 4278 | 0.20029 | 3.51E−12 | 0.94 | G | C | 149 |
| rs27068 | chr5 | 1400239 | T/C | 0.29 | 4276 | 0.07761 | 2.80E−04 | 0.99 | T | C | 150 |
| rs2735839 | chr19 | 56056435 | A/G | 0.14 | 4286 | 0.22415 | 3.12E−17 | 1 | A | G | 7 |
| rs2735846 | chr5 | 1352379 | G/C | 0.49 | 4276 | 0.06895 | 7.14E−04 | 1 | C | G | 153 |
| rs2735945 | chr5 | 1356901 | T/C | 0.39 | 4276 | 0.05534 | 4.22E−03 | 1 | T | C | 154 |
| rs2736102 | chr5 | 1355144 | T/C | 0.39 | 4276 | 0.05553 | 4.22E−03 | 1 | T | C | 157 |
| rs2736108 | chr5 | 1350488 | T/C | 0.37 | 4276 | 0.07446 | 6.48E−04 | 0.99 | C | T | 158 |
| rs2843549 | chr10 | 51191253 | C/A | 0.43 | 4276 | 0.08199 | 1.31E−05 | 1 | A | C | 160 |
| rs2843550 | chr10 | 51191458 | C/T | 0.43 | 4276 | 0.08175 | 1.30E−05 | 1 | T | C | 161 |
| rs2843551 | chr10 | 51191951 | C/A | 0.43 | 4276 | 0.08146 | 1.39E−05 | 1 | A | C | 162 |
| rs2843554 | chr10 | 51193867 | G/T | 0.43 | 4276 | 0.07822 | 2.53E−05 | 1 | T | G | 163 |
| rs2843560 | chr10 | 51182135 | G/C | 0.46 | 4276 | 0.08629 | 2.75E−06 | 1 | C | G | 164 |
| rs2843562 | chr10 | 51166802 | C/T | 0.4 | 4276 | 0.09916 | 1.01E−06 | 1 | T | C | 165 |
| rs2901290 | chr10 | 122997016 | A/G | 0.41 | 4280 | 0.06578 | 3.62E−04 | 1 | G | A | 167 |
| rs2926494 | chr10 | 51187362 | T/C | 0.43 | 4276 | 0.07959 | 1.91E−05 | 1 | C | T | 168 |
| rs3101227 | chr10 | 51190209 | C/A | 0.44 | 4276 | 0.08143 | 1.40E−05 | 1 | A | C | 170 |
| rs3123078 | chr10 | 51194977 | C/T | 0.43 | 4281 | 0.07909 | 2.09E−05 | 1 | T | C | 171 |
| rs35716372 | chr10 | 51159230 | A/G | 0.4 | 4276 | 0.10316 | 1.04E−06 | 0.99 | G | A | 177 |
| rs3741698 | chr12 | 113593606 | G/C | 0.24 | 4277 | 0.07251 | 2.59E−03 | 0.96 | G | C | 186 |
| rs3744763 | chr17 | 33164998 | G/A | 0.4 | 4282 | 0.09664 | 1.90E−07 | 1 | G | A | 187 |
| rs3760511 | chr17 | 33180426 | G/T | 0.35 | 4281 | 0.05741 | 2.74E−03 | 1 | T | G | 188 |
| rs3925042 | chr10 | 123009010 | T/C | 0.41 | 4280 | 0.06741 | 2.67E−04 | 1 | C | T | 191 |
| rs4131357 | chr10 | 51207298 | C/A | 0.43 | 4276 | 0.07794 | 3.61E−05 | 1 | A | C | 196 |
| rs4237529 | chr10 | 122999123 | G/A | 0.41 | 4276 | 0.06611 | 3.36E−04 | 1 | A | G | 200 |
| rs4239217 | chr17 | 33173100 | G/A | 0.42 | 4273 | 0.0962 | 3.03E−07 | 1 | G | A | 201 |
| rs4304716 | chr10 | 51214593 | A/G | 0.43 | 4276 | 0.07968 | 2.75E−05 | 1 | G | A | 203 |
| rs4306255 | chr10 | 51212450 | A/G | 0.43 | 4276 | 0.08058 | 2.16E−05 | 1 | G | A | 204 |
| rs4393247 | chr10 | 123018166 | A/G | 0.29 | 4280 | 0.09239 | 1.83E−05 | 0.99 | G | A | 206 |
| rs4465316 | chr10 | 123024171 | A/C | 0.3 | 4280 | 0.09372 | 6.98E−06 | 0.99 | C | A | 207 |
| rs4468286 | chr10 | 123024381 | A/C | 0.3 | 4280 | 0.09317 | 8.48E−06 | 0.99 | C | A | 208 |
| rs4486572 | chr10 | 51201811 | A/G | 0.43 | 4276 | 0.07875 | 2.33E−05 | 1 | G | A | 209 |
| rs4489674 | chr10 | 123018240 | G/A | 0.38 | 4280 | 0.08456 | 2.02E−05 | 1 | A | G | 210 |
| rs4512771 | chr10 | 51210912 | C/A | 0.43 | 4276 | 0.07991 | 2.46E−05 | 1 | A | C | 211 |
| rs4554834 | chr10 | 51200152 | A/C | 0.44 | 4276 | 0.07753 | 3.71E−05 | 1 | C | A | 217 |
| rs4581397 | chr10 | 51202373 | A/G | 0.43 | 4276 | 0.07778 | 3.57E−05 | 1 | G | A | 221 |
| rs4630240 | chr10 | 51202534 | A/G | 0.35 | 4276 | 0.07404 | 1.19E−03 | 0.98 | A | G | 223 |
| rs4630241 | chr10 | 51202757 | G/A | 0.44 | 4276 | 0.07859 | 2.98E−05 | 1 | A | G | 224 |
| rs4630243 | chr10 | 51210873 | T/C | 0.43 | 4276 | 0.07739 | 4.30E−05 | 1 | C | T | 225 |
| rs4631830 | chr10 | 51213350 | C/T | 0.43 | 4276 | 0.07934 | 2.86E−05 | 1 | T | C | 226 |
| rs4752520 | chr10 | 123001514 | T/C | 0.41 | 4280 | 0.06713 | 2.73E−04 | 1 | C | T | 230 |
| rs4935090 | chr10 | 51161131 | T/A | 0.4 | 4276 | 0.10203 | 9.69E−07 | 0.99 | A | T | 232 |
| rs4935162 | chr10 | 51195705 | G/C | 0.43 | 4276 | 0.07998 | 1.68E−05 | 1 | C | G | 233 |
| rs515746 | chr12 | 113603380 | G/A | 0.47 | 4282 | 0.05828 | 1.55E−03 | 1 | G | A | 238 |
| rs545076 | chr12 | 113604286 | G/A | 0.46 | 4277 | 0.0595 | 1.37E−03 | 1 | G | A | 239 |
| rs551510 | chr12 | 113598419 | C/T | 0.48 | 4277 | 0.06459 | 6.04E−04 | 1 | C | T | 240 |
| rs567223 | chr12 | 113594954 | G/T | 0.45 | 4277 | 0.07814 | 6.74E−05 | 1 | G | T | 242 |
| rs57263518 | chr10 | 51189160 | A/G | 0.43 | 4276 | 0.08371 | 8.23E−06 | 1 | G | A | 243 |
| rs57858801 | chr10 | 51172580 | T/A | 0.46 | 4276 | 0.08625 | 2.96E−06 | 1 | A | T | 244 |
| rs59336 | chr12 | 113600735 | T/A | 0.46 | 4277 | 0.0589 | 1.44E−03 | 1 | T | A | 245 |
| rs62113216 | chr19 | 56056615 | A/T | 0.08 | 4278 | 0.26162 | 1.19E−11 | 0.82 | A | T | 247 |
| rs6481329 | chr10 | 51199752 | G/A | 0.44 | 4276 | 0.07751 | 3.72E−05 | 1 | A | G | 248 |
| rs67289834 | chr10 | 51171310 | T/C | 0.45 | 4276 | 0.08586 | 4.60E−06 | 1 | C | T | 251 |
| rs7071471 | chr10 | 51173341 | T/C | 0.46 | 4276 | 0.08823 | 1.86E−06 | 1 | C | T | 258 |
| rs7074985 | chr10 | 123014878 | A/T | 0.38 | 4280 | 0.08519 | 1.67E−05 | 1 | T | A | 259 |
| rs7075009 | chr10 | 51214149 | T/G | 0.44 | 4276 | 0.07651 | 5.86E−05 | 1 | G | T | 260 |
| rs7075697 | chr10 | 51217377 | C/G | 0.43 | 4276 | 0.07981 | 2.69E−05 | 1 | G | C | 261 |
| rs7076500 | chr10 | 123011721 | A/G | 0.41 | 4280 | 0.06776 | 2.61E−04 | 1 | G | A | 262 |
| rs7077830 | chr10 | 51192282 | G/C | 0.42 | 4276 | 0.08102 | 1.40E−05 | 1 | C | G | 263 |
| rs7081532 | chr10 | 51196099 | A/G | 0.44 | 4276 | 0.07823 | 3.08E−05 | 1 | G | A | 264 |
| rs7081844 | chr10 | 123011258 | T/C | 0.41 | 4280 | 0.06717 | 2.88E−04 | 1 | C | T | 265 |
| rs7090326 | chr10 | 51173381 | T/A | 0.46 | 4276 | 0.08721 | 2.46E−06 | 1 | A | T | 268 |
| rs7091083 | chr10 | 123014747 | A/G | 0.38 | 4280 | 0.0857 | 1.48E−05 | 1 | G | A | 269 |
| rs7098889 | chr10 | 51214481 | C/T | 0.43 | 4276 | 0.07813 | 3.79E−05 | 1 | T | C | 270 |
| rs7405696 | chr17 | 33176148 | C/G | 0.43 | 4273 | 0.10236 | 2.56E−06 | 1 | G | C | 277 |
| rs7405776 | chr17 | 33167135 | A/G | 0.42 | 4273 | 0.10283 | 2.04E−07 | 1 | A | G | 278 |
| rs7501939 | chr17 | 33175269 | T/C | 0.42 | 4282 | 0.09366 | 4.89E−07 | 1 | T | C | 280 |
| rs7896156 | chr10 | 51199385 | A/G | 0.42 | 4276 | 0.08048 | 1.55E−05 | 1 | G | A | 282 |
| rs7910704 | chr10 | 51199811 | T/C | 0.49 | 4276 | 0.07538 | 1.85E−04 | 1 | T | C | 284 |
| rs7915008 | chr10 | 123015215 | A/G | 0.29 | 4280 | 0.09143 | 2.20E−05 | 0.99 | G | A | 285 |
| rs7920517 | chr10 | 51202627 | G/A | 0.44 | 4276 | 0.07847 | 3.05E−05 | 1 | A | G | 286 |
| rs7922901 | chr10 | 123016509 | G/C | 0.38 | 4280 | 0.08614 | 1.37E−05 | 1 | C | G | 287 |
| rs7923130 | chr10 | 123016492 | A/G | 0.38 | 4280 | 0.086 | 1.42E−05 | 1 | G | A | 288 |
| rs8064454 | chr17 | 33175699 | A/C | 0.46 | 4273 | 0.11059 | 8.68E−09 | 1 | A | C | 289 |
| rs8853 | chr12 | 113593290 | T/C | 0.5 | 4277 | 0.07831 | 3.98E−05 | 1 | T | C | 290 |
| rs9630106 | chr10 | 123034373 | G/A | 0.41 | 4280 | 0.08035 | 4.09E−05 | 1 | A | G | 292 |
| rs9787697 | chr10 | 51203382 | C/T | 0.44 | 4276 | 0.07767 | 3.64E−05 | 1 | T | C | 293 |
| rs9913260 | chr17 | 33180010 | A/G | 0.38 | 4273 | 0.1016 | 3.98E−07 | 1 | A | G | 294 |
| rs1016990 | chr17 | 33163028 | C/G | 0.23 | 4273 | 0.09347 | 6.54E−04 | 0.91 | G | C | 723 |
| rs17626423 | chr17 | 33182480 | C/T | 0.2 | 4273 | 0.10224 | 2.81E−04 | 0.91 | T | C | 727 |
| rs2012677 | chr10 | 51174803 | T/A | 0.46 | 4276 | 0.08736 | 2.16E−06 | 1 | T | A | 714 |
| rs2736098 | chr5 | 1347086 | T/C | 0.37 | 4276 | 0.07502 | 6.07E−04 | 0.99 | G | A | 721 |
| rs757210 | chr17 | 33170628 | T/C | 0.36 | 4273 | 0.11727 | 1.51E−08 | 0.99 | A | G | 715 |

Genotypes were imputed in the Icelandic sample set using data from the 1000 Genomes project. Shown are marker identity, chromosome, position of marker in NCBI Build 36, alleles, minor allele frequency in controls, number of imputed cases, predicted effect (in fraction of standard deviation of the distribution), P-value of the association, information content, identities of alleles predicted to be associated with decreased and increased PSA levels, respectively, and the SEQ ID NO for the marker.

### Example 5

We assessed what fraction of 12,779 PSA measurements from 4,569 Icelandic men would be reclassified, with respect to certain PSA cut-off value, after correcting them for four PSA sequence variants, located near TERT, FGFR2 TBX3 and KLK3 (rs2736098, rs10788160, rs11067228, and rs17632542, respectively). For a PSA cut-off value of 4 ng/ml, 6.0% of the men had at least one PSA measurement reclassified; 3.0% moved from below to above the cut-off value and 3.0% moved in the opposite direction. The results for a cut-off value of 3 ng/ml were similar, 6.9% of the men had at least one PSA measurement reclassified; 3.1% moved from below to above the cut-off value and 3.8% moved in the opposite direction (Table 14). If applied clinically, these men would be reclassified with respect to whether or not they should undergo a biopsy.

TABLE 14
Reclassification after genetic correction of PSA levels

| | |
|---|---|
| | Measured PSA |
| | levels after |
| a) Cut-off = 3 ng/ml: | genetic correction |

| Measured PSA levels | PSA < 3 | PSA >= 3 | Total |
|---|---|---|---|
| PSA < 3 | 8,654 | 204 | 8,858 |
| PSA >= 3 | 203 | 3,718 | 3,921 |
| Total | | | 12,779 |

| | |
|---|---|
| | Measured PSA |
| | levels after |
| b) Cut-off = 4 ng/ml | genetic correction |

| Measured PSA levels | PSA < 4 | PSA >= 4 | Total |
|---|---|---|---|
| PSA < 4 | 9,699 | 182 | 9,881 |
| PSA >= 4 | 177 | 2,721 | 2,898 |
| Total | | | 12,779 |

Shown are the number of measurements (n = 12,779) from 4,569 Icelandic men before and after genetic correction, using combined estimates for the four PSA variants (rs2736098, rs10788160, rs11067228, and rs17632542), discussed in the main text.
a) number of measurements that are reclassified with respect to a PSA cut-off value of 3 ng/ml; 143 unique persons (3.1% of the 4,569) have at least one measurement that is below 3 before correction and above 3 after correction and 172 unique persons (3.8% of the 4,569) have at least one measurement that is above 3 before correction and below 3 after correction.
b) number of measurements that are reclassified with respect to a PSA cut-off value of 4 ng/ml; 135 unique persons (3.0% of the 4,569) have at least one measurement that is below 4 before correction and above 4 ng/ml after correction and 138 unique persons (3.0% of the 4,569) have at least one measurement that is above 4 ng/ml before correction and below 4 ng/ml after correction.

### Example 6

### Discriminatory Power of Biopsy Outcome Models

We calculated the area under the receiver-operating-characteristic curve (AUC) to assess the discriminatory power of four models on the outcome of performing a biopsy of the prostate. The four models included the following data: model-1) PSA levels, model-2) the combined prostate cancer risk estimates of 23 established sequence variants, model-3) genetic correction of PSA values based on the sequence variants at the four PSA loci (5p15, 10q26, 12q24 and 19q33.3) discussed above, model-4) the PSA levels corrected for sequence variants and the combined risk estimates of the 23 prostate cancer risk variants. In the analyses of the models, we used 415 Icelandic and 1,291 British men with information on biopsy outcome (i.e. biopsy positive or biopsy negative) and PSA levels, as well as genotypes for 23 established prostate cancer variants and the PSA variants reported above.

### Biopsy Outcome Risk Models

### Iceland

To assess biopsy outcome risk models we selected Icelandic men with a biopsy report and chip genotyped. In addition we required that the individual have an available PSA measurement in the six months preceding the biopsy and furthermore the individual should not have undergone TURP prior to the biopsy. For individuals with multiple biopsies with only negative outcomes (i.e., no cancer detected) we use the first available event. For individuals with multiple biopsies including one with a positive outcome (ie. cancer detected) we use that event. In total 415 individuals fulfills these criteria, 194 of which had a negative biopsy and 221 had a positive biopsy. The median of the PSA level among the 194 biopsy negative men was 8.85 (1^{st} quartile=6.28, 3^{rd} quartile=13.35). The median of the PSA level among the 221 biopsy positive men was 14.00 (1^{st} quartile=8.90, 3^{rd} quartile=25.20).

### UK

To assess biopsy outcome risk models we selected men from the ProtecT trial in the UK with a biopsy report and genotyped using a Centaurus single track assay. We selected men with a PSA between 3 and 10. In total 1291 individuals fulfills these criteria, 948 of which had a negative biopsy and 343 had a positive biopsy. The median of the PSA level among the 948 biopsy negative men was 4.10 (1^{st} quartile=3.50, 3^{rd} quartile=5.10). The median of the PSA level among the 343 biopsy positive men was 4.50 (1^{st} quartile=3.60, 3^{rd} quartile=6.23).

### Variables in the Models

The variables included in the models are (1) PSA value, (2) prostate cancer multi-marker genetic risk prediction and (3) PSA with genetic correction. To calculate the prostate cancer multi-marker genetic risk prediction for each individual we use published estimates of the allelic frequencies and effects of 23 markers associated with prostate cancer (list of SNPs: rs10086908, rs10486567, rs10896450, rs10934853, rs10993994, rs12621278, rs1447295, rs1512268, rs16901979, rs16902104, rs1859962, rs2660753, rs2710646, rs4430796, rs445114, rs5759167, rs5945572, rs6465657, rs6983267, rs7127900, rs7679673, rs8102476, rs9364554). We then calculate the corresponding relative risk for each genotype under the assumption of a multiplicative model at each locus and combine the relative risks for each individual assuming a multiplicative model between loci.

To assess a PSA level after genetic correction we divide the measured PSA level with the predicted combined genetic relative effect. In Iceland and UK separately we calculated the combined genetic effect using the genotypic effects for each SNP as estimated in each population (see Table S3) and combined them assuming a multiplicative model. We selected four markers that predominantly affect PSA excluding the MSMB and HNF1B loci for which we suspect that the association is primarily to prostate cancer (rs10788160, rs11067228, rs17632542, and rs2736098).

We fit four logistic regression models, one for each of the three variables described above (PSA value, prostate cancer genetic risk prediction and PSA value with genetic correction) and one combing the prostate cancer genetic risk prediction and PSA with genetic correction.

We use ROC curves and calculate the area under the curve (AUC) to assess the discriminative ability of each model. Each point in the ROC curve shows the effect of a rule for turning a risk estimate into a prediction of the biopsy outcome.

### Results

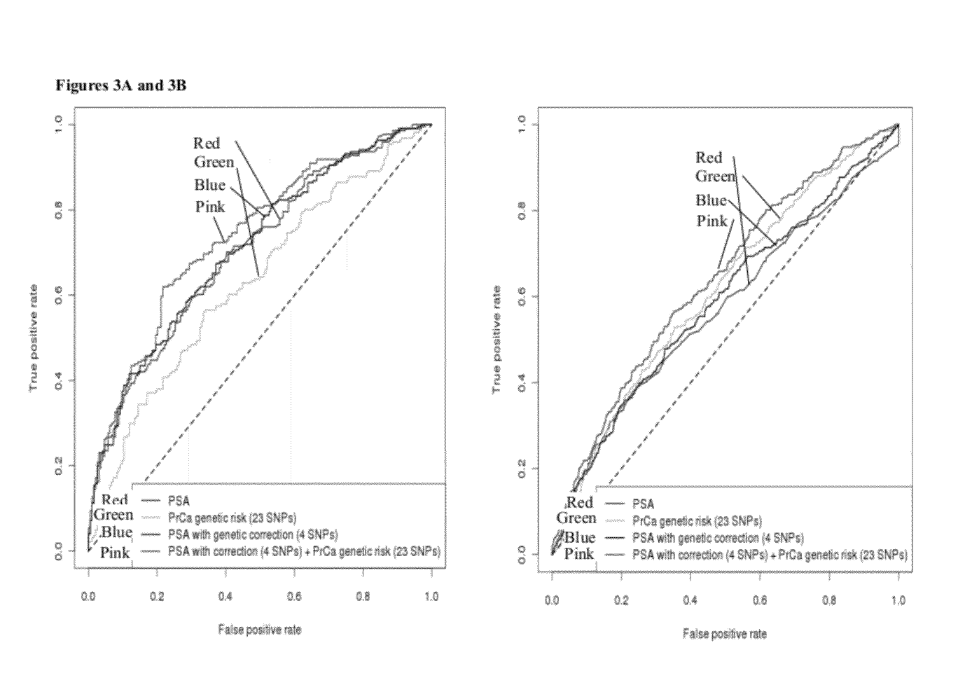

The model with genetic correction of PSA levels (model-3) has an AUC of 70.9% and 58.5% in Iceland and UK, respectively (FIG. 3). When compared to model-1, which has an AUC of 70.4% and 57.1% in Iceland and UK, respectively, the inclusion of PSA levels corrected for sequence variants (model-3) increases the discriminatory power by 0.5 and 1.4 percentage points in Iceland and UK, respectively. However, of the four models assessed, model-4 has the greatest discriminatory power; with an AUC of 73.2% and 63.6% in Iceland and UK, respectively. Compared to model-1 the increased AUC of model-4 is 2.8 and 6.5 percentage points in Iceland and UK, respectively. Hence, the most gain in discriminatory power is achieved by including both the 23 prostate cancer risk variants and the genetic correction of PSA levels. However, in order to better assess the effect of the PSA and prostate cancer risk variants on PSA-based biopsies this type of modeling would have to be done in a population where biopsies are done systematically, irrespective of individual PSA levels, similar to what was done in the PCPT study (3). Nevertheless, the results indicate that genetic correction of PSA levels lead to improved specificity of the models.

1. A method of determining corrected PSA quantity in a human individual, the method comprising:
(a) Obtaining data identifying an uncorrected PSA quantity in a first biological sample from the human individual;
(b) Analyzing sequence data about at least one polymorphic marker from the first biological sample or a second biological sample from the human individual, wherein the at least one polymorphic marker is correlated with PSA quantity in humans; and
(c) Determining a corrected PSA quantity in the human individual based on the sequence data about the at least one polymorphic marker.

2. The method of claim 1, wherein analyzing sequence data comprises determining the presence or absence of at least one allele of the at least one polymorphic marker.

3. The method of claim 1, wherein analyzing sequencing data comprises determining the identity of both alleles of the at least one polymorphic marker in the genome of the individual.

4. The method of claim 1, wherein the sequence data is nucleic acid sequence data obtained from a first biological sample or a second biological sample containing nucleic acid from the human individual.

5. The method of claim 4, wherein the nucleic acid sequence data is obtained using a method that comprises at least one procedure selected from:
(i) amplification of nucleic acid from the first or second biological sample;
(ii) hybridization assay using a nucleic acid probe and nucleic acid from the first or second biological sample;
(iii) hybridization assay using a nucleic acid probe and nucleic acid obtained by amplification of nucleic acid from the first or second biological sample; and
(iv) high-throughput sequencing.

6. The method of claim 1, wherein the sequence data is obtained from a preexisting record.

7. The method of claim 1, wherein the data identifying an uncorrected PSA quantity is determined in a blood sample from the individual.

8. The method of claim 7, wherein the determination is performed using an antibody test for PSA.

9. The method of claim 1, wherein at least one allele of the at least one marker is predictive of an increased quantity of PSA in humans.

10. The method of claim 9, wherein the determining of corrected PSA quantity comprises adjusting uncorrected PSA quantity based on the predicted effect of the at least one allele on PSA quantity in humans.

11. The method of claim 1, wherein the at least one polymorphic marker is a biallelic marker.

12. The method of claim 1, wherein the at least one polymorphic marker is selected from the group consisting of rs401681, rs2736098, rs10788160, rs11067228, rs10993994, rs4430796, rs2735839 and rs17632542, and markers in linkage disequilibrium therewith.

13. The method of claim 1, wherein determination of the presence of an allele selected from the group consisting of the C allele of rs401681, the A allele of rs2736098, the A allele of rs10788160, the T allele of rs10993994, the A allele of rs11067228, the A allele of rs4430796, the G allele of rs2735839 and the T allele of rs17632542 is indicative of elevated PSA quantity in the individual.

14. The method of claim 1, wherein determination of the presence of an allele selected from the group consisting of the T allele of rs401681, the G allele of rs2736098, the G allele of rs10788160, the C allele of rs10993994, the G allele of rs11067228, the G allele of rs4430796, the A allele of rs2735839 and the C allele of rs17632542 is indicative of reduced PSA quantity in the individual.

15.-22. (canceled)

23. A method of diagnosis of prostate cancer in a human individual, the method comprising:
(a) Detecting an uncorrected PSA quantity in a first biological sample from the human individual;
(b) Obtaining sequence data about at least one polymorphic marker in the first biological sample or in a second biological sample from the human individual, wherein the at least one polymorphic marker is correlated with PSA quantity in humans;
(c) Determining a corrected PSA quantity in the human individual based on the sequence data about the at least one polymorphic marker;
(d) Determining whether the corrected PSA quantity is greater than normal PSA quantity in humans;
(e) Performing a further diagnostic evaluation procedure selected from the group consisting of rectal ultrasound imaging and prostate biopsy on the individual if the corrected PSA quantity is determined to be greater than normal PSA quantity in humans;
wherein determination of a positive outcome of the ultrasound imaging or prostate biopsy is indicative of prostate cancer in the individual.

24. The method of claim 23, wherein the obtaining sequence data comprises determining the presence or absence of at least one allele of the at least one polymorphic marker.

25. The method of claim 23, wherein the obtaining sequencing data comprises determining the identity of both alleles of the at least one polymorphic marker in the genome of the individual.

26-46. (canceled)

47. A method of determining a susceptibility to prostate cancer, the method comprising:
analyzing nucleic acid sequence data from a human individual for at least one polymorphic marker selected from the group consisting of rs17632542, and markers in linkage disequilibrium therewith, wherein different alleles of the at least one polymorphic marker are associated with different susceptibilities to prostate cancer in humans, and determining a susceptibility to prostate cancer from the nucleic acid sequence data.

48-57. (canceled)

58. A method for identifying a human individual who is a candidate for further diagnostic evaluation for prostate cancer, the method comprising the steps of:
a) obtaining data representing uncorrected values of PSA quantity in the individual;
b) determining, in the genome of the human individual, the allelic identity of at least one allele of at least one polymorphic marker, wherein different alleles of the at least one marker are associated with different levels of PSA quantity in humans, and wherein the at least one marker is selected from the group consisting of rs401681, rs2736098, rs10788160, rs11067228, rs10993994, rs4430796, rs2735839 and rs17632542, and markers in linkage disequilibrium therewith;
c) determining a corrected PSA quantity in the individual based on the allelic identity of the at least one polymorphic marker; and
d) identifying the subject as a subject who is a candidate for further diagnostic evaluation for prostate cancer if said corrected PSA quantity is greater than values of normal PSA quantity in humans.

59-64. (canceled)

65. An apparatus for determining corrected PSA quantity in a human individual, comprising:
a processor;
a computer readable memory having computer executable instructions adapted to be executed on the processor, wherein said instructions comprise steps of:
(i) obtaining data representing uncorrected PSA quantity in a biological sample from the human individual;
(ii) obtaining sequence data about at least one polymorphic marker in the genome of the human individual, wherein different alleles of the at least one polymorphic marker are predictive of different PSA quantity in humans;
(iii) determining a corrected PSA quantity based on the sequence data about the at least one polymorphic marker.

66-69. (canceled)

70. A computer-readable medium having computer executable instructions for determining corrected values of PSA quantity, the computer readable medium comprising:
data indicative uncorrected values of PSA quantity for at least one human individual;
data comprising sequence data about at least one polymorphic marker in the genome of the at least one human individual, wherein said at least polymorphic marker is predictive of PSA quantity in humans; and
a routine stored on the computer readable medium and adapted to be executed by a processor to determine corrected PSA values for the at least one human individual.

71-72. (canceled)

73. A method for determining the prognosis of an individual diagnosed with prostate cancer, the method comprising
(i) detecting an uncorrected PSA quantity in a first biological sample from the human individual;
(ii) obtaining sequence data about at least one polymorphic marker in the first biological sample or in a second biological sample from the human individual, wherein the at least one polymorphic marker is correlated with PSA quantity in humans; and
(iii) determining a corrected PSA quantity in the human individual based on the sequence data about the at least one polymorphic marker;
wherein the corrected PSA quantity is indicative of the prognosis of the individual.

74. The method of claim 73, wherein the method further comprises determining corrected PSA velocity by repeating steps (i)-(iii) at least once, using a first sample and/or a second sample taken at a different time than the first of said first and/or second sample, and calculating a corrected PSA velocity based on the corrected PSA quantity determined for samples obtained at the different times.

75. A kit for determining PSA levels in a human individual, the kit comprising
(a) reagents necessary for determining the quantity of PSA in a blood sample from the individual; and
(b) instructions for correcting the PSA quantity determined in (a) based on the genetic composition of the individual.

76. The kit of claim 75, wherein the reagents for determining PSA quantity comprise at least one antibody selective for PSA.

77. The kit of claim 75, wherein the kit further comprises reagents for determining the identity of at least one allele of at least one polymorphic marker in the genome of the individual.

78-80. (canceled)


SEQUENCE LISTING (727 sequences)

SEQ ID NO: 1 - DNA, 401 bp, Homo sapiens
tcacaaagagctggggcagcatgaagaggaaaccaaaggcatagacccctgcagaaagacagacagcactcacgaggtgcggagggccgggctgccacatctacgcacagacggcactcacgaggtgtggagggccgagctgccacgtctatgcatagtgggcagaaaacaaggtctgctatccagacaacttcagagtcyatcatggtgtgaagcagctttctggctggtaagtttatcaagagtctacgacaactttggagagcaaagctcttctatttattaatcagtgtaactgctgcccacggggtcaagtcagtgagagcactggagcaccctggggctatgaggacacggcctcctgacccacaaggtcctgccccaggggtcaggtgtgggac

SEQ ID NO: 2 - DNA, 401 bp, Homo sapiens
aggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcrggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggat

SEQ ID NO: 3 - DNA, 401 bp, Homo sapiens
tgataagaaaactctcatctggtgggcttgtggcttcgatgtgtacttagccaaaagggcggccacatttctttgggtttccttctcattaaccacaagggaacgagcccagagttgaattcagataaacctgaatgcaattcccagtctgatcatcgctggttacaagaactcaccttaataattgaatctcatggccargtgtgatggctcatgcctgtaaccccagcactttgggaggctgagacgggcagatcacctgaggttgggagttcgagaccatcctgaccaacatggagaaaccccgtctctacttaaaaaatacaaaattagctgggcatggtggcgcatgcctataatcccagctactcgggaggctgaggcaggagaattgcttgaac

SEQ ID NO: 4 - DNA, 401 bp, Homo sapiens
gcaaagagaagttctctcctcctctgctcttttaggtcagcagcaaatgcagatcggggtggggacaaggtaaacacataacttgggtggagatgtgattgattaataactcataaatcatctgaaaccatactttccttttgatagtcaaccctctgtaaacactcaatgtgttctcaccttgttatcattcccaatgaygtcgaatgcgtggttgccctctccagtataaaagtttgatgcagctttgcctggatgtacctgtctataaggagtcctgcttatcacaatggtaggtaactggttttatatataaaggctagaaaagcaaaagagaatatgtattcctattttaatatgcgtgtgcttctaccacatagcattgaatggtaaatgtgacc

SEQ ID NO: 5 - DNA, 401 bp, Homo sapiens
agtaagcctgccagccccctgctgccctgactggcgtgagcaacagcaataaataaataagaacagagacaagaagcttatgtactttggagctcctggttctatttgatgtcattggacaccagtttgcttttctttaaagcctgtttcttttgaactgggctgccgcagtctataaaattttaacgcatggatttcccrttgttcgggtgtctgtggggcaccagccttcctgtttcactgtggtgtgccgaccaggaaagaagctggctggctgggaggtctgggggtagttaccttgtttctggatatgcatgtgattgtgttcactttagcagatgggatctcagaccccacaaataatcataatagcaggcgtgcatcaaacttactgtcggagt

SEQ ID NO: 6 - DNA, 401 bp, Homo sapiens
ataaataaataaataaaacaaaaataacaataacaacaatctcttgaagaggctgttgtatttgtaaaacgtcccttcctcagcatcttgagaaactttctggacctctgtcttttgagaagtttccagtctgcctttcctgcctccttctcctttctgaagaaattctcattgaatacagagaggcagcacagactggaratgctgcataaagcttaaattgggcagggcccaagcgttgttgggtctttggagacaatggctcctgagaatttttttaggctttccaggaactacagagagttgcttcatgtcaggaacacaaattcttaaagagctagtcaccaagtatggggggccaacccattcttggaaaggtctctcgctctaagcagcaaaca

SEQ ID NO: 7 - DNA, 401 bp, Homo sapiens
gtataccaaggcacttgggccgaatgttccaagggattaaatgtcatctcccaggagttattcaagggtgagccctgtacttggaacgttcaggctttgagcagtgcagggctgctgagtcaaccttttactgtacaggggggtgagggaaagggagaagatgaggaaaccgcctagggatctggttctgtcttgtggccragtggaccatggggctatcccaagaaggaggaattcagaaataggggaaggttgaggaaggacactgaactcaaaggggatacagtgattggtttatttgtcttctcttcacaacattggtgctggaggaattcccaccctgaggttatgaagatgtctgaacacccaacacatagcactggagatatgagctcgacaag

SEQ ID NO: 8 - DNA, 401 bp, Homo sapiens
ttgaaccagaggagtgtacgcctgggccagatggtgcagccgggagcccagatgcctgggtctgagggaggaggggacaggactcctaggtctgagggaggagggccaaggaaccaggtggggtccagcccacaacagtgtttttgcctggcccgtagtcttgaccccaaagaaacttcagtgtgtggacctccatgttayttccaatgacgtgtgtgcgcaagttcaccctcagaaggtgaccaagttcatgctgtgtgctggacgctggacagggggcaaaagcacctgctcggtgagtcatccctactcccaagatcttgaggggaaaggtgagtggggaccttaattctgggctggggtctagaagccaacaaggcgtctgcctcccctgctcccca

SEQ ID NO: 9 - DNA, 401 bp, Homo sapiens
aatgctgttggctggaagacgcccttgctcacgctctgctcagtgagaagggattcctcaccggctgtcacactcaccttgtaggtgaaggccttccagggcagatgtgccactgacttcaactgaaacaggagagacaggcccaggtcagctgtgggcagcacaggagacgggggccgggccgcgggcagcacacaccgycttaccttgtagttcacaaagagctggggcagcatgaagaggaaaccaaaggcatagacccctgcagaaagacagacagcactcacgaggtgcggagggccgggctgccacatctacgcacagacggcactcacgaggtgtggagggccgagctgccacgtctatgcatagtgggcagaaaacaaggtctgctatccaga

SEQ ID NO: 10 - DNA, 401 bp, Homo sapiens
atgggctgggttcacccgcctggtttccactgagagggccccgtggggccggggtctgtctgtagcctgactcgggtgcttggtgctggagtctggaagcatgcagggcttgttgagctgcacggggtgcatctgccatgacactgtgtggaaattgtatagcaacaagttttttaaaagtgcctgccggcctggtgcagyaactcatgcctgtaatcccagcacgttgggaggccaaggcgggcagatcacgtgaggtcaggagttcaataccagcctggcaacatagtgaaaccccatctctactaaaaatacaaaaatgagccgggcgtggtggcagatgtctgtaaccccagctactcgagaggctgaggcaggagaatcgcttgaacccgggtggc

SEQ ID NO: 11 - DNA, 401 bp, Homo sapiens
agcctcccaggtagctaggaccacagatgtgtgtcactatcaccatgcctggctaattttaaacatttttgtagggacacggtctccttttgttgcccaggttggaggacagtggcatcaacatagctcactgcagccttgacctccctggcccaagtgatcctcctgcctcagcctcccaaatggctgagaccacaggcrcacaacaccatgcccagtgaacttttatatgtttttttgtagagacaggcttttgccacgttggccgggctggtcttgaactcctgacctcaagtgatctgcccacctctgcctcccaaagtgctgggattacattcatgagccaccgtgcccggcctgtctttgctatttgccaccagctctctgtgccctctgtgcac

SEQ ID NO: 12 - DNA, 401 bp, Homo sapiens
atctcactctctccctgcttttacccttagggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaacyccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctgg

SEQ ID NO: 13 - DNA, 401 bp, Homo sapiens
tttttaatttttcttttgttatttgtatttgctagtctctgatttcctcaaaacgaagtggaatttactactgttgtcagtatcggtgttttgaattggtgcctgcctatagagatatattcacagttcaaaagtcaggtgctgagagatggtttaaagacaaattcatgaaggtatattttgtgttatagttgttgatgrgttctttggttttctgtatttttccccctctctttaaaacatcactgaaatttcaataaatttttattgaaatgtctttgggccttgtgttaaatgttttttctttgggaacctttcctgaagatggacagtcaggggagggtttagtatcttcttgttctgagtttacccccttcccttcgcctttaaataattaagac

SEQ ID NO: 14 - DNA, 401 bp, Homo sapiens
caaatagggaaggaactcactcagagaagagaatataatttaaaaatacttttcctcctacgaggggaacaagtagttattttgtcacattaaaaacaatggtttcttgcaatgcatcaaagacataaatctgaacacaccagacctcttcattatccctgtggcctaacaaactcctaaaggcaaagggagccagtttcygagcaggccaagggattcaggctgtacgctccacaggggacagaaggctaaatcagccggaaaaaggtagagctgtacagttgggtaagcacaagtgcagacaagcgtggataattttaactcccattacagctggactgaccactaaactgtcacctcatatcgagcttgtaactgagaaaccacaattatccttttga

SEQ ID NO: 15 - DNA, 401 bp, Homo sapiens
tttatacacattatatttaaacagtgaacttgaattctttagtacatagcatacatagatcaagtcttttgtgataacaaatgctaaactagagaagcaagaaatgcccaaattccagaagcctgtattctaaaaattcatttgtaggccagttatttgaaatcaaaagtttaatggaaacataacaataaatgatgctgytcatgggtctaatcaaacctactgtagccctcccataattgaaatgcattttgtttgaaataatatataaaacattagcaaggactcatagttttaagcaactaaaagccatttaaacacaaagaatactcaacactgtcttaccaaatgtagagtcatttcctggcatggtcttatactgttaattatcagagcaaaaa

SEQ ID NO: 16 - DNA, 401 bp, Homo sapiens
ataactgttctgggtgatccaacatgcgtgagggtttgttcaggctgtttagagttcttttgtgaaactgtcaaaacaatgacctcatttggagccatctgctggcatgcaggattggcacggagctcgctgctgatgtgtgtcgctggggcaatcaattcaagatggccactctgggcacaccgtttactaagcgatatsggagcagataactgattgctccatctcttcctcaagttactaaacatgttagtgctcacacagcctccaccaaaggctgtgtgccaggcatggcaaagggaagacatcatctgctgacccagaaacacaaacccctacatttcctggagtggagggtgcttctcctgccaggtcctggcctgaacattaacttgcaaagc

SEQ ID NO: 17 - DNA, 401 bp, Homo sapiens
ccccgtctctactaaaaatacaaaatattagccaggcgtggtggtggacgcctgtagtcccagctacttgggaggctgaggcaggagaatggcgtgaacccagtaggcggagcttgtagtgagccgagatcgtgccactgcactccagccagggtgacagagtgagactccatctcaaaaaaaaaaaaaaaaagaattacwctatgtcctccaaaaacataagtgaaaatattgacagaagtaccatacacaataccccaaaactataaacaacccaagtgaccatccacagtggaacagatgcattatggtagtatcagaccatggaatactataaagcaataaaaatagaaaatactgctatatacaacaacatagatgaagcttacagactaaatgct

SEQ ID NO: 18 - DNA, 401 bp, Homo sapiens
tggtggtaaattttgctaagtccacttacaacattacaggctgaaatcctaactttatggaacaatgccagtggttctgttgtttctatacagaattgaaatactatcaattaatagacactttctgcccattcattcattcaaacatttatcagggctgaccacacaccagacaacgtggtaggcactgaagtcagcaawgctcagactttagcatgaagaatcacctgtaagatttgttaaaagggcagattctccaggctcagaaatcagagttggctgggtgaggctccaggtggttctcatataggtggtcgctagacgacacgttgatatatgtgaggcttcggagatgaaagatgaatttagttttatttgtttatgtctgccttccttcacta

SEQ ID NO: 19 - DNA, 401 bp, Homo sapiens
tgtttctttcctgttgtattagttggctcaggctgccataacaaaataccacagactgggaggcttaagtaacagaaattcatttctcacagttctgggggctggaagtccacgatcaaggtgcaggaaaggcaggcttcattctgaggcccctctcttggctcacatgtggccaccctcccactgcgtgctcacatgacytctttgtgctcctggaaagagggtgtgggggacagagggaaagagaaggagagggaactctctggtgtctcgtctttcaaggaccctaacctgggccactttggcccaggcactgtggggtggggggttgtggctgctctgctctgagtggccaagataaagcaacagaaaaatgtccaaagctgtgcagcaaagacaag

SEQ ID NO: 20 - DNA, 401 bp, Homo sapiens
ttttttatctgtctacatctataatcactatgcatactagtctttgttagtgtttctattcaacttaatagagatatgttatacttaaaaaaaaaaaaaaaatgggccgggcgcagtggctcatgcctgtaatcccagcactttgggaggccgaggtgggtggatcatgaggtcaggagatcgagaccatcctgattaacrtggtgaaaccccgtctctactaaaaatacaaaaaaaattagccgggcgtggtggcaggcgcctgtagtcccagctactcaggaggctgaggcaggagaatggcgtgaacccgggaggcagagcttgcagtgagccgagatggcgccactgcactccagcctgggtccagcctgggcaacaagagtaaaaccctgtctcaa

SEQ ID NO: 21 - DNA, 401 bp, Homo sapiens
ctgcccaaagaatttatgatttgttccaggcaaaacctgaatataggcccccgaacccccttttcctaaaggatttactttagaaaactggcagttagaaatcctttctctgtctcttggaaatgtatcttctataacacaggaatgtctttcccaaggacctgggagccatcctttcgaaatgtaatcattaggtaggasagagcctctgtctcccaatcttggtggtagggtaggaccctgatattgataagcaccagttcacaatttgataagcacaagttcacaatctggtcagtgtactcattgaccaaacctccctcccctcaactagcatccttcagtacttttcccttaacacatcctagcatttaaaaagcctcttgccttctggtttggta

SEQ ID NO: 22 - DNA, 401 bp, Homo sapiens
tacatgttgatggatttttgtttgctaatatcttattgagtatgtttgcatctctattcatgagagatattgttctataatttttgtttattttggtgtgtttgtttagctttgatattggggtaacactggcctcattaaaggagttggaacatgatctatccttttttattttctgaaagatttttgtgaaagacatawtatttcttctcgtcttattgtgtcgtgtcgtgtcctgtcctgtctttttgaggtggagttttgctcttgttgcccaggcgggagtgcaatggcatgatctcggcccattgcaacctccacctcccaggttcaggtgatgctcctgcctcccaagtagctgggattacaggtatgcaccaccagacctggctaattttttt

SEQ ID NO: 23 - DNA, 401 bp, Homo sapiens
tacatatgtctctgtcaaaattgggataaagagaaacagactggcagtggtatgcatttcaaggaaaataggagaaagcctatttacttaggaaagtgaggctcataagtacttcatatacaaacatcatacctgcttcactgggttgtgttacctgacttgccccgagcatttgaacttgctacccccaatagagaattkactccctcattagccaaagaggaatctgagacctggtgagggtgagcacctggccagtcatcagtaagctccttttcatcccacccaccggcggtgattagtaaccatgtctgacaggcatttactttcctcctggtgagtagctggaaaagagagtagtttggaaatatttggaaatagcaccgtttgtctctcacata

SEQ ID NO: 24 - DNA, 401 bp, Homo sapiens
tcatctttcccttccagctttgtcttgtcctcagaaccacacaaacttaggggactcatcatcccttgcgtgaccatgtgttctcctctggccctctgggttccagactaaatatttctgggtccgtcaatgatcattcatagtttcccaaaatcctactactcttctctaggctgctctactttatcaatgtccttagartttggctccatagcagagcccctcactgtggtttgtgcatgcaactgaccacaccctgtgctgatgtgtggtctgaccagcacaaaagggtcacctctcttgagccagacactggatttctatgattgcatctcaaaagcatgctcactttggcttttatacacattatatttaaacagtgaacttgaattctttagtac

SEQ ID NO: 25 - DNA, 401 bp, Homo sapiens
attatcataagggatacaaagggaaaataggataagtagggttaggatgtaatagttactattactattgttgtctcttggacgcttgggtggggaatctactctagaaggggtatttcagggaaggcacttgtgtcctgaaagatgggatgcatgcagctgtgggaagagcaagtgcattccaagtagagaggcagtacmtgcaaaggccctgagaccagtgtgattgcagcaccaaaggcatcaggtggcacaagatgtggctgaagagctaggtcaaggccaggtcatgtgtgtggccttaaagatcttgctatggagagcatctcatgcataccccaaatgtctcatgcgtggtgcctgacacatagtaggtgctcagtaaaagattagtgaatcct

SEQ ID NO: 26 - DNA, 401 bp, Homo sapiens
caggtaagaagacgggagaggaggtgtcacctccgacaagccagtcacagcttgcttcatagactgaccaaaaatcaggtagagagacagtgcgttccccagctgcagatggtgtcttagagctgtgccatggagcccatgtcttgacaaatgacaacagaggcagaagaatgggctgtggggagtagcaaagaaccagarcctcccctgcatccttggggacagggagatcaccaggggcagcaactgtcgcaggcagacgtgagtccagagcgggaggagtgtgaaataggacctaacagaggggaattctgccccttggagatcctgagcctaggaacgtaacactccttaatcttgcaataacataatttaaaacaaacttttattcccccttagag

SEQ ID NO: 27 - DNA, 401 bp, Homo sapiens
tcaattctgtggttaatgccacctagggatataattacacaagctaatatcatctttctactggaggagaggaaaagagattctcatgggcaatattcatttgaattaaccatcttaggtttaatactaagactacaatcaaattcctaaggtattcttattgaaaagtttaaaattaaaaaattattcatctgatagtarcattaacatgcttatagtatatcttatccagtttattaaatgtccatttcagaagtgttctcaaaacactccctcccacataattaaaaggattttgcttaaaataaaccaattgtattatgaccctcacacacagcagcagaatttagggagagttcaaacattcgatgaagcactggtgctccttattgggtctcaag

SEQ ID NO: 28 - DNA, 401 bp, Homo sapiens
aagcagtcagggagctgtaatcatcatctatgtgttgccttggctgcagaggtcagtgttggaaatgagaggtgagaatgcaagacccttaccaaagaaacaaaaaagaaagctacaacaacaacaacaacccttattgaaatgcacacaatccaatcaaatccaataaaaatagtatgcaccaaaagagccataaaaaakgttcatgttctttggctcagtcatccctgtttttaggaatttatcctaaagaaaagcaaaccactgtgacatgatttataatagaaaaaaaaaacaaaaaacaactggaaacagcatcaacatgcaacaacagtaattggttaattaaattatggtacatccttttgatgaaatgttatgtctccaaaaagactaagtat

SEQ ID NO: 29 - DNA, 401 bp, Homo sapiens
acacacatacttgaaactatatacatatagtacatatatggagtacgtatatacctatagtacgtagatatatgatactacacacacacgcaacacagcctatcagtgcagtttctctgaagaaccctgaccagtatagagctctgcttccataattgttgcctgtgctgtcacctggtgcagaggtgttctgaggacatytgttgaatgaggaagggaggagagagctgctcttgcgtcctggaatgggggcgctctcaggccagttgcccatatccctctgagaaactcatgtaaattaaaacactggcaaaaaattattaaagttataactcacagagctcctgcctctgcgtgcctgcctctgcgtgtctttgctaatattttcaacgtttgttaag

SEQ ID NO: 30 - DNA, 401 bp, Homo sapiens
aaaaaccaccagtatactaattccccaggaaagcaaatcctatgtggcccacaaaggtggcatcatttcaaagtgccaaatatccacttacatccctgcagttgtccttgcaccatttccctctcaggtaactttaatcaagtctgttaacatcaaccatcagctcacttccttaagccatatctgctactagattgaggkttcttaatcctaacgaccatcatcaaattgcaatcatctggggagactttcccaaagcactattcctgagccccaccctagaccagttaaatcagaattgacaggaatggaacctgggctgattctaatgaatcaggcaaaataaatgctaagacccacatgacagacgggctttctaaagcagacctttagaatcagat

SEQ ID NO: 31 - DNA, 401 bp, Homo sapiens
caaaatgccacagttttcttattcttgttccacatattggtattgatacctcctaatgatgtaagtgcaacagaccccccaggttacaagtcacagtaacctgagcatgtgcagatgaacccagcatgcctgattagtgaccctggagcaggctggaaagaaaaattcaagcacatgtgaaatgtaagtaccgaacccagraatggggaccaaattaagaagtgaggggtgcccccttttgttgcagtatgaacttaaaagtcaaggacccagcaccacctctctgcatagcccaaatcaggtcatgcctccttgcatttccttagctcccttataattactctcagcctataaaacctgtccccagaccccagctccgggagacagatttgaggtttcct

SEQ ID NO: 32 - DNA, 401 bp, Homo sapiens
cacatccaggaaaggataagccagaactgattcgcgtctcctcttctatctgatactttagggcaatttaaaatgtgttatctgacaatatagaaaggagaacattagagtggccatgctgaagaatgttaagaaatcatcgttttacaacagattatccagtcctgacttggtggaatcaggacttctgtctttatttgwaaaagtccaccaccaacagttagcattgcctcctagagccaatagcctgtgggtactggagagggaggggtgcccggctgctggggtgccacagtggccacggtgatgtagattctctctccattgggcttgcccaatacgtttcacgggacgtctcccctaatctcttgacctgcctctagttcctcatggaggtggag

SEQ ID NO: 33 - DNA, 401 bp, Homo sapiens
tatttgaccaaaataaaacattaaaaaagaaatgtgtgtatgtgttttaaactttttcagctattttagcaaggtaggtttacatgaagagtatgctatttggaaatatttattcgtccattcattactcatttcacagtaggggaaacattcctgtttctcccccgtggtggtgataagtggaaatagcttctttgaacrtgtgcagggtttcactgaccttggcccacccctgtgacgtggggcctcagtcagcgaagtaccaactctccgggagtctatggactcagcctgcagagcctggaggagccacttctggtggacatggggtgcagaaggaaacatcagactcatccaccctgcccgtcacctgcaggttttctaaagtgtggaggagtgtg

SEQ ID NO: 34 - DNA, 401 bp, Homo sapiens
tccacccatcacgtttgtgtttaaggaatgtataggtgagaccaacgagcgcaggcagcgtttcttttgtgctggtccctttcatttgaaacaaaatgtcttccccagaagctctagaggaaatttacgtctaatcagtcaaaactaagtcacattctattctcaaggaaggctgagaaagagagtatttggttttccagsctcttcaggaggaggttactggggagaaggaaattgggggcaccttgtgagtaatcacctcatgtcctccacccacttccccctttgggactttggaatagaaaggggagattaggaaccactgtttgaccactgcttccccgaggagcatggacaatgggaaaggatgcaaggtctgaatccctcatagggatactgta

SEQ ID NO: 35 - DNA, 401 bp, Homo sapiens
ctgcctggcctcccaaagtgctgaaattacaggcttgagccactgtgcccagcatctgtctctctctctcccctcacccccacgcatatacacttatacacttttttttaaaacaatttgaaagtaatttgcattcatcatgtccctttatctatgaacacttcaatgtggagttcctgagaacaaggactttttttaatrtgatctcagaacatttatcaaaaatcagaaaactcaatgttgataaaataatattatgtaatccatagtccatattcaaacttcaccagttgtcccaatattctatgttcaagtccaggatcacagattacatttactcatcagatttctttaattacctttcatgtgaaacagctcctcaacctttctctctcttcagt

SEQ ID NO: 36 - DNA, 401 bp, Homo sapiens
tattatgtaatccatagtccatattcaaacttcaccagttgtcccaatattctatgttcaagtccaggatcacagattacatttactcatcagatttctttaattacctttcatgtgaaacagctcctcaacctttctctctcttcagtggccttgatatttttgaagagtttaggccagttactttgtaaaataggtttrtctgcagtttccttgtgattagattaaattctgcatttaaaaatattttattttacttatttttgagacagggtttcactctattgcccaggccagagtgcagtgatgcagtcatggctccattggagcctcaacctcccaggctcaagtgatgttcccacctcagcctcctgagtatctgggactaaaggtatgtgcca

SEQ ID NO: 37 - DNA, 401 bp, Homo sapiens
ttccagataccaaggggtttttatccaaatagttaagtaatattttaatcttttttctatcttaacataagcaagacaaaaatctgcactattggtggttttatattaggatattccagaatatccccattatgcaccctattatgcaccccaacacatagaggaaaaaaacataaaggtagtcatatattgttctatttyattaaatgttgctttcttttactttgggttgcagactttggtatgtttttattaaagtataataatggcaaaagcttctggttctgatggaatcacctgtagtagatcttagcaggagtgctccctagaaggttagataagatacaaaaaaaaaggaaaaaagaaaaaaaagatacatttaaaggaatcagagaactgcc

SEQ ID NO: 38 - DNA, 401 bp, Homo sapiens
actttgacctagtctattctaggccacgggggttgggttttcctcattctgaatttgctgattaaaacacatgctttcacactcacacacatgcacactctcacacacacactcacatgcacacacacatgctcacatacacacacattcacacactaatttatacacacatgcacacagcggcaatatagttcctttaaycaagaaaaacaaatgttccatctggaagaccatttgcaccagtcagtaaggagtcgggtcgaggaagcaggttgcaagcccccctgagcatcctgctctgtcttcacggcttgtcggatgctgacctgaggaccacaccatggaaggcaggtgagagatcagcccagggatgcaaacctgtttctgtgtcttccttttat

SEQ ID NO: 39 - DNA, 401 bp, Homo sapiens
tcacacactaatttatacacacatgcacacagcggcaatatagttcctttaaccaagaaaaacaaatgttccatctggaagaccatttgcaccagtcagtaaggagtcgggtcgaggaagcaggttgcaagcccccctgagcatcctgctctgtcttcacggcttgtcggatgctgacctgaggaccacaccatggaaggsaggtgagagatcagcccagggatgcaaacctgtttctgtgtcttccttttatggtgtctgccataccggaagcctgtcccaggtcacttcctcattggtcactaacaagtctagaggagatacaaagtcaaccattggctgccttcagtcgggaggagtctgagactggcaactgctattggcccatgcactgtcacatg

SEQ ID NO: 40 - DNA, 401 bp, Homo sapiens
ataaaagaggacatgcgtttctactgcatgaagtaggcatggattttaaaaggcagaggaattattgcactcaggaactctttgaggcagaaagaaagtcattactgtgcctgcaggaagagctgggcattcagtacacaatgagtgaatgaatgagtgaatgaatgaatgaatgaatgaacaaatgggtaagtaaatgckgcgctcagagcgctgatgtcctgtgcgctcaggtatccaggcagagagaaaggggcacctgggagatgggattctatgactgagtttgcctctgacagtgcaacaggtaagaagacgggagaggaggtgtcacctccgacaagccagtcacagcttgcttcatagactgaccaaaaatcaggtagagagacagtgcgttc

SEQ ID NO: 41 - DNA, 401 bp, Homo sapiens
agacagtgcgttccccagctgcagatggtgtcttagagctgtgccatggagcccatgtcttgacaaatgacaacagaggcagaagaatgggctgtggggagtagcaaagaaccagagcctcccctgcatccttggggacagggagatcaccaggggcagcaactgtcgcaggcagacgtgagtccagagcgggaggagtgkgaaataggacctaacagaggggaattctgccccttggagatcctgagcctaggaacgtaacactccttaatcttgcaataacataatttaaaacaaacttttattcccccttagagttgggccctgagcaaaaacaacatagaaagtcatcaacaccaatgtctaaacaccacttgagaaagagttctactcggcacaag

SEQ ID NO: 42 - DNA, 401 bp, Homo sapiens
gggactggaaagaagccagctaaaaaagccctcttaacctaagaaaaccctcttaacttctgtccttccatggaacaaaaccaaaatgacataccgcccccaaaacattgtcccctctgatggtgactaaagaatacaatccatgctagggtgataaactacaaacataagctcattcaacaatgttttaaaaagttgtcytttttaaaatagctatttctcttatgttccctaatccttacaagtaatttaaaaatcgagtaattaaaaattaatatactccatgaaacatggattaaataacaaccaactaatcagaaccctcactccattggaatttttgcaacctctgttgtttaaataaacattaatgataagaaaactctcatctggtgggcttg

SEQ ID NO: 43 - DNA, 401 bp, Homo sapiens
cctacactctaatttccacccttcacatgtagcatgattatacaacagagctgtacacgcatcaagtcaaaaaccaaggcttgtttgccagaaggaaaccaaaccttcaattctgtggttaatgccacctagggatataattacacaagctaatatcatctttctactggaggagaggaaaagagattctcatgggcaatrttcatttgaattaaccatcttaggtttaatactaagactacaatcaaattcctaaggtattcttattgaaaagtttaaaattaaaaaattattcatctgatagtaacattaacatgcttatagtatatcttatccagtttattaaatgtccatttcagaagtgttctcaaaacactccctcccacataattaaaaggatt

SEQ ID NO: 44 - DNA, 401 bp, Homo sapiens
ctggggagactttcccaaagcactattcctgagccccaccctagaccagttaaatcagaattgacaggaatggaacctgggctgattctaatgaatcaggcaaaataaatgctaagacccacatgacagacgggctttctaaagcagacctttagaatcagatgaaataaaccttctcctctgaaagctcccccaccaatyattccaggccacatgcttcactgctcaggtaatgagagggtctgggctccagaggttcagccactgctctctcaagatacttttctaaaacgacaaacctcccaaagaattttccagagtttcacatgtgcaacagttgaaactatgttgtgttgggttgtttttttttttcccctgaaaacacatgtgtccttttgttt

SEQ ID NO: 45 - DNA, 401 bp, Homo sapiens
ttataattactctcagcctataaaacctgtccccagaccccagctccgggagacagatttgaggtttcctcctgtctccttgatagtcaaccttgcaagaaagctttctcttttgtcaaaagccagtgccatagtgttggattctatgtgcattgagcagggaacccattgcttggtgacaattaccataatgatgataaygtcaggaacagcatacagaaataacgactttcccaatgctatggagatccttaggttcaagagctgaggtcttctcctcctccagttccctttctactttatcacacatttctttgtcatttccataagatcaagatgaccttggcttaagtaacagctaagaaggcagaccttggagccccgctctgtaggtttaaatc

SEQ ID NO: 46 - DNA, 401 bp, Homo sapiens
tgacctgctctcagcctccggttgcttacctgaaaaatggatccccattgagtttctacagtgaaagagccatggagggcctaagcctcagagcctggtcaagtatttgccatctatactgcctgttttccctctcatataattccctttcatcatttgatctgttcatcatgcactcttgcagagaccatttcagaaacmtctccaaaccaggcactgacacagagcacttctccggttctatctgttccaaacatttcccatatttcaccttttttttttactcttccacattttcaagttttgaagatttgttcacaatacaatgatttagggttctggatctttatttatgagccctcctgcctcttgcccagatacagggtgtggaatatgctctt

SEQ ID NO: 47 - DNA, 401 bp, Homo sapiens
agcctcagagcctggtcaagtatttgccatctatactgcctgttttccctctcatataattccctttcatcatttgatctgttcatcatgcactcttgcagagaccatttcagaaacctctccaaaccaggcactgacacagagcacttctccggttctatctgttccaaacatttcccatatttcaccttttttttttamtcttccacattttcaagttttgaagatttgttcacaatacaatgatttagggttctggatctttatttatgagccctcctgcctcttgcccagatacagggtgtggaatatgctcttaagttgcctgcagagacacagttaaccagagcttgcacatgtgtgcatgcacacacacacacatacacacacacacccatcca

SEQ ID NO: 48 - DNA, 401 bp, Homo sapiens
gatttagggttctggatctttatttatgagccctcctgcctcttgcccagatacagggtgtggaatatgctcttaagttgcctgcagagacacagttaaccagagcttgcacatgtgtgcatgcacacacacacacatacacacacacacccatccaggtacacatcagagacctgtcagcaggtaattatcttccatgcygatggctctgaattgctgactgccttccagctacattcactcttcaaacttccaacttcacttcctgtcctctttgcagcggtttctgacttcaggagccctgaggcagtagacctgctcagccctttctgtcttgcctcttcttgcccctataaagatttgctgccctgagctatacaatttccagagcaatcatgtaa

SEQ ID NO: 49 - DNA, 401 bp, Homo sapiens
tacagggtgtggaatatgctcttaagttgcctgcagagacacagttaaccagagcttgcacatgtgtgcatgcacacacacacacatacacacacacacccatccaggtacacatcagagacctgtcagcaggtaattatcttccatgctgatggctctgaattgctgactgccttccagctacattcactcttcaaactkccaacttcacttcctgtcctctttgcagcggtttctgacttcaggagccctgaggcagtagacctgctcagccctttctgtcttgcctcttcttgcccctataaagatttgctgccctgagctatacaatttccagagcaatcatgtaagatactttacaacccagcccagtccagaagcagaaagctatctttctgtaa

SEQ ID NO: 50 - DNA, 401 bp, Homo sapiens
ctgcagagacacagttaaccagagcttgcacatgtgtgcatgcacacacacacacatacacacacacacccatccaggtacacatcagagacctgtcagcaggtaattatcttccatgctgatggctctgaattgctgactgccttccagctacattcactcttcaaacttccaacttcacttcctgtcctctttgcagckgtttctgacttcaggagccctgaggcagtagacctgctcagccctttctgtcttgcctcttcttgcccctataaagatttgctgccctgagctatacaatttccagagcaatcatgtaagatactttacaacccagcccagtccagaagcagaaagctatctttctgtaaagtcagtccaactgttgtcttattattagt

SEQ ID NO: 51 - DNA, 401 bp, Homo sapiens
ctgagctatacaatttccagagcaatcatgtaagatactttacaacccagcccagtccagaagcagaaagctatctttctgtaaagtcagtccaactgttgtcttattattagtattttattggccaaagagtgcttgcccagatatagtggggctatttttacccaggctgtccctttgcaggtgcggcaggcaatgcarcttgaacctgtgtggtttcatctaacgcaggggtagagggaaaagagtgggcttggtgagttcattcacctatgggatcctccttgtccctcttttatcttctctgtgctatcttcctttctttgctctgatgaatactcctggtggcttggtacgagcgtaaagccagagaggttatgtagccgaaattcctaagaagg

SEQ ID NO: 52 - DNA, 401 bp, Homo sapiens
ggccaaagagtgcttgcccagatatagtggggctatttttacccaggctgtccctttgcaggtgcggcaggcaatgcaacttgaacctgtgtggtttcatctaacgcaggggtagagggaaaagagtgggcttggtgagttcattcacctatgggatcctccttgtccctcttttatcttctctgtgctatcttcctttcyttgctctgatgaatactcctggtggcttggtacgagcgtaaagccagagaggttatgtagccgaaattcctaagaagggagaactatttcactgtgcacttacagatatgttgtttcactgatccacagtaagtgcacagtgcaatagcatatctgtgcacatcagtagcatatctgactctattaccagtagaaaccac

SEQ ID NO: 53 - DNA, 401 bp, Homo sapiens
gtaagactgtgtttactaatttgtgttttcttcctagacaatctgttcccagtaaccattctatcattgaaactggaggtaatgcttacaattttacttaataaaaagtctatcagaagacttggtccacaggggtagagttggtagtgccaatgccacacacacacaaagcccccaggaagggctaaccaatgcttgtgytgttgctcttgttgttggacaggagatatgttaaattagaatcagaagagaataattactttcatacatagtagaatatcagaggtggaaggggccacaggctcatctagtcaaggaatggacatgtacgccatgcacgctttgctctctcccccatctagtacccatggcagacatcaccaatcattcatcctttccaa

SEQ ID NO: 54 - DNA, 401 bp, Homo sapiens
cccccaggaagggctaaccaatgcttgtgttgttgctcttgttgttggacaggagatatgttaaattagaatcagaagagaataattactttcatacatagtagaatatcagaggtggaaggggccacaggctcatctagtcaaggaatggacatgtacgccatgcacgctttgctctctcccccatctagtacccatggsagacatcaccaatcattcatcctttccaattggcatcctttccaattgaactcagaggtgatctcaaaatgcctcaccaagtcactaccaatcaatcacagttgacatgtaagatcatacacatttgccatcacttagcccctcattttactgatgaaacagttgagactgtgagggcgggaacaatattattcaacact

SEQ ID NO: 55 - DNA, 401 bp, Homo sapiens
aaaccccttcagtggggatagttcattaaaatcaaatagcttgttacaacaggttttcagctgaattgcttaatgtatgttttataaaacttctttacaaggcatctgccaatgtttagccagaatgttgggttggtgtgttggttttcctcttcagggaagattcaagaaaatggtaatgggatgtgaagacaattatayagaagcatgttgttgaagactggaaaaataaatgaagagctattaaatctgctagaattggagccaggctctaactatgcgtagaaacagactggcttcggccccatcaaggcacgttgagatcaagcccatgaaggatgcatcagcttccattataattacagccactgctctcagttttacatagttctcactcctga

SEQ ID NO: 56 - DNA, 401 bp, Homo sapiens
tccttctacgtgcctttcaatcctatttcacagtcccttccaaaaggaccacaaagcatggcaaacagagatagactcagtctaccaagtggctacaaaggatgttgttttataaagcagagaacctgagccaagcttctgcacaatacagaggaagagtcaaggctctagacagactggtccaggccatggtgaatggcsaggcccattcaatcccaaccatcagaggagcttttttcctttccaaagcatgagtcctacagggatgaagatgagaatctccttgaatttagaattgtaatgtctcaactggaggagttccaaaagattccctactattagctccaggaagcagaggctgggccagatgaaatggattgcccacgattctcagcgagttg

SEQ ID NO: 57 - DNA, 401 bp, Homo sapiens
caactttggggtagacatgaggccaaaatggaagctaaacagaggagaaggtgactgcccctgtacggtacacctcatccctttcttccgaggttagtgggcaaaagaggacttcccatcttcaggaataaaaaagcaccaccaccactctctcttattaagcaaaaagaaaagctcttgttttatttaattaattaattwatttatttattattattttttttgatgtggagttttgctcttgtcacccaggctggagtgcaacggcatcttggctcactgcaacctccaccaggatcctgggttcaagcgattctcctgcctcagcctccagagtaactgggattatagatgcccagcaccacacccagctaaattttttatttttagtagagatgggg

SEQ ID NO: 58 - DNA, 401 bp, Homo sapiens
gagatcacaccactgcactccagcctgggcaacagagtgggactctaggcaacagagtgagattctgtctcaaaacaaaacaaaacaaaacaaaacaataaaacaaacaaaaaataccaacaacaaaagaaaaaagaaaaagaaaatgacaaacaactggattaaaatgaacaccttttgctttactaagagacaacaaayagagtgaaagaacaagccacaccatgcaagaagatatttgcaacaattgataaaggattagtattcagaatttaaagaatgtgaatgaatcaatgtgcaaaagacaaacacccaaatcaagaatggacacagaggcatgaataggcacttcacaaaggagggaacatgaatgacccaaaatcctgtgaacagatgaacaa

SEQ ID NO: 59 - DNA, 401 bp, Homo sapiens
actgcttggaccaatagagtacagtggaagtgacactatgtgacttccaaggtgatatcataaaaggctctacaatttctaccttgctcactggaaatactcactcttggagctctgggccacctgtaaaaacccaagaaccctgaagctgccatgctgggaggaagcccaggtcatacgaggaggctacatgcagggtckccattcacagtcccagctgagcccagcttttgggccctcctgcccaggagccacacttgtgagtggagcagcctccagataattccagtgcccaatccttcaagtcatggggccccagacatgaggagccctcctaactcgccctgagtgcctcatcctcagaacctgtgagcgtaataaaatgggcctgttctgtgtct

SEQ ID NO: 60 - DNA, 401 bp, Homo sapiens
gagacagtgtcagagccggtggtgagagtcgtgcgactttagcgcagggatgctggcaggcccagacgggagtccctggatggggttctgagctccaggccaggctgggaaactggagggaggtaggaggagcaagctcaggtggcttgaggtggcctagagaggtgcccaggcccagcagggtacattgcagagttcctstttggcgggaggtgagagcgcttccaatgcacatgcccagaactgggcccacagaagttgaagcgcaggagagaggtgaggacactgagtactggatgggggcctggaccccacttgcagcttcaaggggatacagggtgggctgccgcatcttgagctagggggaggcttggatctgtgtgtcctgcgctgtgggtttg

SEQ ID NO: 61 - DNA, 401 bp, Homo sapiens
aggtgcccaggcccagcagggtacattgcagagttcctgtttggcgggaggtgagagcgcttccaatgcacatgcccagaactgggcccacagaagttgaagcgcaggagagaggtgaggacactgagtactggatgggggcctggaccccacttgcagcttcaaggggatacagggtgggctgccgcatcttgagctagrgggaggcttggatctgtgtgtcctgcgctgtgggtttggcagagagactggagtgggtgtgaggtggtaaaaagcaagtctatgggggaggactgtggcgggtgacccagacggggagccgggtttgggcatggcctcttctgggtatggatggacctggcctcctcccctcctgagctgggcaggcagggctctcagag

SEQ ID NO: 62 - DNA, 401 bp, Homo sapiens
gcagcttcaaggggatacagggtgggctgccgcatcttgagctagggggaggcttggatctgtgtgtcctgcgctgtgggtttggcagagagactggagtgggtgtgaggtggtaaaaagcaagtctatgggggaggactgtggcgggtgacccagacggggagccgggtttgggcatggcctcttctgggtatggatggrcctggcctcctcccctcctgagctgggcaggcagggctctcagagctccggccatggcagggactctctgcccgcagtgggaagccagccttcagggtccatgctctgcccagccttgctgtgcaggctcagcctgtcacattggattagcctgaccctaagtccccagcctgtcccagtcctgccaccagagccagttc

SEQ ID NO: 63 - DNA, 401 bp, Homo sapiens
cggggagccgggtttgggcatggcctcttctgggtatggatggacctggcctcctcccctcctgagctgggcaggcagggctctcagagctccggccatggcagggactctctgcccgcagtgggaagccagccttcagggtccatgctctgcccagccttgctgtgcaggctcagcctgtcacattggattagcctgacmctaagtccccagcctgtcccagtcctgccaccagagccagttctggctgccccagcccattttgtctgttttaacagcagctgacaggctatatcccccagccagtctgcagatcagtgttcagaccgtgcatgcacagccatcagggctgcctctccagcctttggtctttttgtttcaagggcacaggaagtcaaaga

SEQ ID NO: 64 - DNA, 401 bp, Homo sapiens
atttttacatatcggccttatatacttgaaccttgtttgctaaactcatttattaattccagtaaaattttttgatagattacttgggattttctgcatacagatcatgtggtctacaaataaacacagttttacttttttcttttcattctagatgtctttaatttacctttctctctctttctccctctaccccctttytaagttgccctggctatgaccagtagtacaatgttgaatggaagtggtaagagtagacattcttgcctgtttcctaatcttgagtgggaaacattctgtctttcatcattaagtacattaactgtaggttgtctgtagttgccctctatcagattgaagaagtttacttttattcctagtttgttgagagtttttaccac

SEQ ID NO: 65 - DNA, 401 bp, Homo sapiens
ctaccttgggcacatgttgtcagaacctcctgagggtgtgtcacggtagcatatcctcaaccttggcgacttggtgctaacgctgtactaaattaccaggctggttaacatctattggaatttcttggtgccaaaggctgtgggagacctgaacacattgtcaagggaggaggaaggggaaggagaagggctgtgggatcycccgcatgaaaggccccatgctggtggtagcccccacctgctgctctggggctcaaaggtgcttagatctcttcacttgagtgcagatgctgccagccctttgggcaggtgtgtgagggaggcagccctggctgtaaagagagcaggggcttggctgggcgtggtggctcacgcctgtaatcccagcactttgggaggcc

SEQ ID NO: 66 - DNA, 401 bp, Homo sapiens
tgatcctctgaagagaatgaattgggacagtctagaaaggcatgaagcccgtaggtatttctgaattgtgaccgagtttgcctagcacagcgttttcacagcctgtgcaattagagagcaggtgtgttttaagtgctcatgaaaagaagagccctcatttttattgcaagttttccctcatttctctgagaaacaaaacaragcaactcaaggaatatgcaggaaaattgtttttgacccaatttgttgtcgttgccaacttactttccaaagtggttaattaggcaagctcagcagaactttccagtttctgcaagggtgccagtttcggagcacctctgtagactccctctgctgagggatgagatggtgacacttttctcagatttccttttgtttat

SEQ ID NO: 67 - DNA, 401 bp, Homo sapiens
caatctatttaaatgccttctggtgccgctatactgttcttcatttttgtctcagcaactcggttgctcctagattctctttcagccccagccttcctccattgcctcatattttactcacaggactgagctgttgaaagatttgtggagcaatatctatatgttattttaaaagcagtttaggtggaacaaaaatcctcrtttgagcctttgttgttctcaggaattaggtaaattgagctcagaaacagatttcagcaccaaaaaagattgtttagaacatcttttaaaatccaattctgacagagacaatttattgctttggttaagcaagcacagtcttgttgaccctgctgcagtcttaccttctatcaagaaagggcaaagaaagctccgtggac

SEQ ID NO: 68 - DNA, 401 bp, Homo sapiens
ggttctgttgtttctatacagaattgaaatactatcaattaatagacactttctgcccattcattcattcaaacatttatcagggctgaccacacaccagacaacgtggtaggcactgaagtcagcaaagctcagactttagcatgaagaatcacctgtaagatttgttaaaagggcagattctccaggctcagaaatcaragttggctgggtgaggctccaggtggttctcatataggtggtcgctagacgacacgttgatatatgtgaggcttcggagatgaaagatgaatttagttttatttgtttatgtctgccttccttcactagactgtaagattctctagaccaggagttggcaaatattttctataaagagccagatagtaaacatattaggc

SEQ ID NO: 69 - DNA, 401 bp, Homo sapiens
tgcaaacctgtttctgtgtcttccttttatggtgtctgccataccggaagcctgtcccaggtcacttcctcattggtcactaacaagtctagaggagatacaaagtcaaccattggctgccttcagtcgggaggagtctgagactggcaactgctattggcccatgcactgtcacatgatcctcttttctggcagccagtkggggatgaacttgatggaaaagagaattgagacagacggaccgctttgagcagccactgctagaagcaggatgatgaagggctggctttgtgggacatttctggaaaaaatcaataagagttcgtgactaactagatggtaaccagagagcagagaaggaaagaaaggcattctacccaattatgtttttagattaggag

SEQ ID NO: 70 - DNA, 401 bp, Homo sapiens
cagccctatcctgttcaacattttgattagtgttacaaatgaagacattgcttcatttattcaacaaatgttcattgagcacatattctgtgccagacacaacctggaagaaaagcttagcaaaccaatggatgagataaagctggaaggggtcgagattaatcctcaggcaggggagctaaacccatcagaattttttawaaacagagctagggccaacttagcatattgtcaagccaaagtgattgttttgtggcccaatgaattagcaaatgatagtaagtgttaactccaagtacttccaggaaagtgccagtctgaatgagaagaaagtctgaataagtaattatttttaacgacaagtagtttaataactctcaagtagtaaaataattcatact

SEQ ID NO: 71 - DNA, 401 bp, Homo sapiens
aaatactgaggggatagacatggcgtaggcattccaagtacttgttatcaatccagtatctatctgggtagaaaaagaaaatgaactgggcttcattttctcaggtaggctagaaaatgaagtggtccttactcagccactaaccagtgcctgccatgtgctgggtcaccttggccctgacatttcctagttagttctagrtgttttggagaagaggaaacaaatccaggttgtcatactctgaaggctcttgaatttggaagagtctgtggaggaggtcagagggggaacagaaagcaactcataagctggaaacacagcctaaaattatcattgctttgcttttagtcacaatagttttttgctggtttttttttgtttttttgttttgttttgttttt

SEQ ID NO: 72 - DNA, 401 bp, Homo sapiens
gaagaatgggctgtggggagtagcaaagaaccagagcctcccctgcatccttggggacagggagatcaccaggggcagcaactgtcgcaggcagacgtgagtccagagcgggaggagtgtgaaataggacctaacagaggggaattctgccccttggagatcctgagcctaggaacgtaacactccttaatcttgcaatamcataatttaaaacaaacttttattcccccttagagttgggccctgagcaaaaacaacatagaaagtcatcaacaccaatgtctaaacaccacttgagaaagagttctactcggcacaagcattcccaggtcctagagagctcccagacatcatttattctcaccagggaggcagccccacactccctcacgaggtctctg

SEQ ID NO: 73 - DNA, 401 bp, Homo sapiens
gccctgagcaaaaacaacatagaaagtcatcaacaccaatgtctaaacaccacttgagaaagagttctactcggcacaagcattcccaggtcctagagagctcccagacatcatttattctcaccagggaggcagccccacactccctcacgaggtctctgcgtggaaattcctgtactcagggaagacccctagtcggtrgctgaaccatgcggactatgacagctcttcttgttttttgtacagctccccgccaccgccccccaccccaaatgtgccatttcgagtggctggagtacaaggtacaagtagaagaaccactagaaacagcattggaaaagtttcctggtcatgaggtggagcgcttaggacaggcaagtaaaggagcatgttaatcactc

SEQ ID NO: 74 - DNA, 401 bp, Homo sapiens
actggagatacagatattttattttcactttttcaaatgatagttaataacttgctgctaagagacatgtgtagaagggtccagcatgacttccctgccactactttggacatgggtgccagccagcccagcaccaaccactctggagatgggatggacattggcaatgtagagagtaggaaaacctggattctaattctrtctgccaccagctacttctatggccttctctgatctgttacactgagacttgagcttttcgtgttcaaatggcttctccagcctttttgattgttccaaaatcaaaaccattaatatcctgaaagaagaaggaaaaggcctctggctcttttgccctggtgctttgcataactcagataagatacaggattctctgccgg

SEQ ID NO: 75 - DNA, 401 bp, Homo sapiens
aatatagtctcattcaagttcacataatagcagaagtgagagaaaaccacccacatccctgccttccatgggtaggctccatatgtcaagacataggaagacataatattgcctgcctttatatctgcaaattgaagagctaattcatttctaaggaaaaagaaatacatttggcactgaaatggaaaataccaggaatgytggaaaattccagtgccagttctaaaatgatgacagggtacacaaatgtgttctggctggttaaattttgcaaatttcagaaaaagatgaatggataataagcatgattcttgcttatggacccccatggaaaagtagcataatatcggtattacattacctttcatttccatcagcaatgagttacactatgtcagtcc

SEQ ID NO: 76 - DNA, 401 bp, Homo sapiens
aggtcccaaactgatgtcttcacttgcctgactcagaaaccatgtcttctgcaatgactttaatggtacaagtagtatctgttacctcccctgagagccagactcttggcctgacatccttaatagttaaattagaccaccatttcctctggctcaacttttattcctgaacaagtcatactggttgcatggtcaaaggtyggattatacagaaagtcaataggtgcttttgaggctaaagagacaagcacctgtcctactttaaaagcatccattcctctcttgttttaagattgaaatatacctggaagggtggttactaacctctagggttcacttgcccaggaagcaggagaccatgatctgaaaaatcatgtcatcacttccatgtgtacatgact

SEQ ID NO: 77 - DNA, 401 bp, Homo sapiens
caaacaagattgctgggagtcacacttaagccactcaagagagacttcagcacacagcttacatgcaaacgacagtgctccttatggaggcatcttaatcgtccttaaagcagaactggcaaggcttgttagcagtagttcaaatgtctatagaccctttcatttcttttaaaactcttttgtgttgaaatcttccactcrttccaattgtttgttctctgttggtccaatcaaatgttgtctaacatgctgtttaggtgcaaaagaatgaagagtaaagaacctatctgccgagccttaaaaaaaggctgctccccagcactttgggaggccgaggctggcggatcacaaggtcaggagatcgagaccatcctggctaacactgtgaaaccccgtcgcta

SEQ ID NO: 78 - DNA, 401 bp, Homo sapiens
gtgattctgatgtttgccaaaatatgagaaccattgctttatgttaattgacttactttgttatttcacgcccactatctataagctaattcccactgcagtgaggcagagagagatggctccacactgcactcttaggtacaatcgagcgtccccatttggtgttgagaggtctgctttattcttcccatttatttgatkggcacgtttttaacttagaacaatatttctctccaaacagtgtgagcatcttattgtaagtttttacaaacatttttttagccatttcaggtaacaacccccagcgtaacaatcccccacgtggcctgtaacacccagaactctggtgaatgtaatgattgttttgcagacaggaagtgactgcagcattaacacgatcc

SEQ ID NO: 79 - DNA, 401 bp, Homo sapiens
tctctttatatatatttttagccattcacatttcctttctaggaactgttgttcatatcctttggtcataatttttaaagttttctctttcttgtgtatcagcagagtcccttacattctagatattgttatctcgttggtcctgggcagtgcaaatacattttcctaatctctcatctgtctgttagctttttctatatygtctttttaaaacaaaagtctttacgctttgtgtaatcagaatcatcaattattcacctaatggtttgttctattcaggtcatgttaaagttcttcttttcctggaggtcctatattttcttctattagcattctggttttacttttcacatttaagtcttccatccatcaagaatctactttgtatatggtgtaaggta

SEQ ID NO: 80 - DNA, 401 bp, Homo sapiens
gtcctatattttcttctattagcattctggttttacttttcacatttaagtcttccatccatcaagaatctactttgtatatggtgtaaggtagggctctaagctttgtttttctttgtattgtgagccaattttcttaacaccttctattaaatcatgtgtcctgttgcccattgacctttatcatctcaagctgccatrtatctaggtatcagttataaattatttctgttcaattggtctacttctctgttcttgtaccactaacacatagtttttagttctatagttgcatactctatcttaatatctgatcacagaaatttgccttcttttgcttcttttccaaagtaacttagctattcatggacctatactcttctgtatacagaatagctttc

SEQ ID NO: 81 - DNA, 401 bp, Homo sapiens
ttccatccatcaagaatctactttgtatatggtgtaaggtagggctctaagctttgtttttctttgtattgtgagccaattttcttaacaccttctattaaatcatgtgtcctgttgcccattgacctttatcatctcaagctgccatgtatctaggtatcagttataaattatttctgttcaattggtctacttctctgytcttgtaccactaacacatagtttttagttctatagttgcatactctatcttaatatctgatcacagaaatttgccttcttttgcttcttttccaaagtaacttagctattcatggacctatactcttctgtatacagaatagctttcttacattcattcctaaatccactggaattttgttctggatcaccctgaaact

SEQ ID NO: 82 - DNA, 401 bp, Homo sapiens
ctaacacatagtttttagttctatagttgcatactctatcttaatatctgatcacagaaatttgccttcttttgcttcttttccaaagtaacttagctattcatggacctatactcttctgtatacagaatagctttcttacattcattcctaaatccactggaattttgttctggatcaccctgaaactgtacattagtwtagagagaattggcatgtttatcatcttaaagcatcccatttatatacatggtacagtttgccttttattcacattttcttaaaatattgtccatagatactatgtattccttgttaatttctagatattttataatttttgttgccattatgaaggcatcttgtcttttatttactggttactggtggtagggagagat

SEQ ID NO: 83 - DNA, 401 bp, Homo sapiens
gcttcttttccaaagtaacttagctattcatggacctatactcttctgtatacagaatagctttcttacattcattcctaaatccactggaattttgttctggatcaccctgaaactgtacattagtatagagagaattggcatgtttatcatcttaaagcatcccatttatatacatggtacagtttgccttttattcayattttcttaaaatattgtccatagatactatgtattccttgttaatttctagatattttataatttttgttgccattatgaaggcatcttgtcttttatttactggttactggtggtagggagagatgtaattaatgcttccaagttgatctgtgtcttgcagatttcctgagctccctaatcattttcctgattttgac

SEQ ID NO: 84 - DNA, 401 bp, Homo sapiens
cattgtggttttatttttgagacagagtctaactctgttgcccaggctggagtgcagtagtgcgatcttggctcactgcaacctctgcctcccaggttcaagccattctcctgccccagcctcccgagtagctgggactacaggtgcatgccaccacagccggctaatgtttgtttgtttgtttgtttgtttttagtaaakatggggtttcactatgttggctaggttggtttcgaactcctgacctcggcctcccaaagtgctgggattacaggtgtgagccaccgtgcccggcctcactgtgattttgatttactttttgtcagtggctaaagatgttgagcatcttttcatgggcttgttggccatttgtataacactggaacatgttggttattgag

SEQ ID NO: 85 - DNA, 401 bp, Homo sapiens
tacccaccacaaaaatgaccacccaaaccaaaagtcagcatagtgcagtggtttaaagatgggaattctggagccggattgcccgggttcaaatcacagcactaccacttaaaaagattgtcattgcgcaagttacttctccactctgggtcttgttacttcatgtgcgtattgagactaatactagttcttacctcactvagcattaactattctatcaccgtttaccctatatccaatctcagcaacaataggtttgtccaatatatcaagtcgaaatatctactaaccagttccatatcagaatgattgcttgtgctcacaatgatgaatgtaaaaaattacaagcacctaaaacaattcccgatttaagaatggaattctgttcggcagaacattaa

SEQ ID NO: 86 - DNA, 401 bp, Homo sapiens
aaaaattacaagcacctaaaacaattcccgatttaagaatggaattctgttcggcagaacattaatgttccgtgaattttattgagtgcagaccctgcatattcagaatctgtacagggcacatatggaaacttaattaactgaactctttaattcagcagaacaccaactgccaaccacattggctacctgagggttaawggcagagagagaaagctagagtctccattatctgatattgtgttgatatatgcatcatctgagtcacaacacagattacttaagatgtttacattaaggaataaaggaagttctagattgcttgtgaaagtattagaaatatgtcacgtcattcccatgaccacagccaccatatgatcacatgctaaccacatcaatcc

SEQ ID NO: 87 - DNA, 401 bp, Homo sapiens
tgggaggctgaggcggcggatcacgaggtcaggagatcaagaccagcctggccaacatagtgaaaccccgtctctactaaaaataccaaaaaaaaaaaaaaattagccaggtgtggtggcgggcacctgcagtcccagctacttgggaggctgaagcaggagaatggcgtgaacccgggaggcggagcttgcagtgagccragatcgcgccactgcactccagtctgggtggcagagcgagaccccgtctcaaaaagttaaataaataaataaataaataaaacaaaaataacaataacaacaatctcttgaagaggctgttgtatttgtaaaacgtcccttcctcagcatcttgagaaactttctggacctctgtcttttgagaagtttccagtctgcct

SEQ ID NO: 88 - DNA, 401 bp, Homo sapiens
gggagagcgacgagggaagaacctcaaacctatgaattataccaaagtgtgtttggggaaaaaaaatctacaagttcccctgattgttttggctagagggcaaaccgccaggccctcggagcacaaattcaaatgcaaggtcatatcccaccccaaaccgccgctgcggacgtgcggccaaggctcagccccgaaatcgcrgcgcttcagtgtccgaagccacgcgatgggctctgtcctaaagcggagtggcccgggagcgacgtgctgtctcagggaaccgctcggctccttagataattagtaaccagctgtcaaagtctagggcggtcggggaggacccacaaacgatcccggccctggggatacactgccagccggcctgaactcccggggagagg

SEQ ID NO: 89 - DNA, 401 bp, Homo sapiens
aaacatctgccatagggatgaaaggaacttacctttactgaagacctactatgtgaccagtgtggtgctctacaatgcattggtcatcctctaagaagaaaagaatgatccctgagaccctaccctatgggtaacacttaacaaatcagcccacagctaagagataagtgaaaaccaaggcaagtagatacagacctcccrggggcaggattaagaaggaaaagaggcaaggagagttttcaggagagaccacagcgctggaggcaggaaagtaagatgtaaaagagatgctcctagacctgtcaatcaagcttgcctgaactcgtcacactaagaagagtaatggcataaaaagccaacctgaggaccgccctggagacacttgagggacaactgctgaa

SEQ ID NO: 90 - DNA, 401 bp, Homo sapiens
ctgcctcccaagtagctgggattacaggtatgcaccaccagacctggctaattttttttttttttttttttttgagacaaagtcttgctctgtcacccaggcgggagtgcagtaacgtgatcttggctcactgcaagctccgcctcccgggttcacgccattctcctacttcagcctcctgaatagctgggactacaggcrcccgcgaccacacctaactttttttgtatttttagtagagacggggtttcaccacgttagccaggatgatctcgatctcctgacctcatgatccacccgtctcggcctcccaacgtgctgggattacagttatgagccaccatgccctgcctaattttgtatttttagtagtgatggggtttcaccatgttgatcaggct

SEQ ID NO: 91 - DNA, 401 bp, Homo sapiens
aatatgaagtcacagtaattatgacttagtcctgttcgccaatttccaccttccatataatgtcgacagtataaagacaatatactcattgtttctctaacatgggattaatattttgtctgcaatagacaagacctccctcttttcagagtcctttgtgatgtaccctattaatcagtaatacgttaaagacttctcacrtttcagaatgtataacagcttatgattgagttactatttaattttctgcccctgatttttgaggattctaatcccaaagtttagtccaagctacacatttttggtagttgtttaatgcccttataaagtttttcttttaatggtaataacaaaaggtctgattttaacactaccttttacgtaactggtttaaattagca

SEQ ID NO: 92 - DNA, 401 bp, Homo sapiens
tgcctgaaggacacacgtccatgcaggcaggatttagaaatgtataccacccttgagctccaagaatcgattctagttgacaaggtcttagagcatcagtgggaacatttcactgtccaggctagatatttcttcattttcttgcaggcaattcggcagttagaaaatttcaactccaggtttaagcacctgctgtccccmtggtggtcctgtagggggaacagagctgggcgtgagcctcagagagctctgtgtgcagactctgcaaggccacacctgccctgcctcttggagcaagttgctcacctgctctgagctccaggtccctccctggtgcaatgagcatagtaagccctcgccccatggagttctgagaactgtgtgggatcctgcgtgtgcag

SEQ ID NO: 93 - DNA, 401 bp, Homo sapiens
tctcatttcgtattgttcccagagagtgatggggggggtcttaatttatttgggctcacacacttctgagaatcggagataaattgtaggatcccgtcgagaaatgtgcacctctgcacaataacaaacttgcgtatgatttcaggagattcacgggaagtcctgagccctctcttcctgttagaagtcctagccctgggmctaatccccaattagctcaagggtgccggctctggaggagtgggtgccctgggggcggggtgggcgggggggaagccgggtgtcggttggggtggtgggtgcgccatttggcagatacatattcagcgctggtgcttctaacaggctcccactgcggctgtatgcctgggttgggagggttggaggttgagggtgactaa

SEQ ID NO: 94 - DNA, 401 bp, Homo sapiens
ctccagcgcagtccgcagcgcgttcgttaaggagcccaacttcggcttcttagaatgcagcccactttctagacgcccccggcgcacctgcctcgaggccgcgagaaaggccagctccggccgggagccggggaccgggaagcgcgcagtcccgaccttcctcccctgcctttctcttccaggaggtttactagaactccrggaactccaaaacgcgacttttctttctctttgcttttcgtcttaagggaacacggtgggcaaaagaagggagtgcgtgtgcccgaggcttgggagccccctgtgcagtgacctaggaaccgctagcgatttcttgaaaaattactttaatcaagtccggcgctcccgggagcgggggaggaaaggcagcacccgctcgg

SEQ ID NO: 95 - DNA, 401 bp, Homo sapiens
caagcagatggttagggtactagtgggagacatctggggagaaacattctttttctagggggtgctacctatggtctatgcaaaattctgaattttccccccatctaagctacaactttggggtagacatgaggccaaaatggaagctaaacagaggagaaggtgactgcccctgtacggtacacctcatccctttcttcygaggttagtgggcaaaagaggacttcccatcttcaggaataaaaaagcaccaccaccactctctcttattaagcaaaaagaaaagctcttgttttatttaattaattaatttatttatttattattattttttttgatgtggagttttgctcttgtcacccaggctggagtgcaacggcatcttggctcactgcaacctc

SEQ ID NO: 96 - DNA, 401 bp, Homo sapiens
aatacaatgttgtatctccttgggacgggaggatcacaccactgaggtgaggcaagtagttaaaaaaaaaaaaaatccacagctccaccttccagacaaaatctactgggctaatggggacaaataacttcaatttcggacattaacaattttagatgcttgttttcctgtattatgtttctcataaattttaaatactgrataagaacatggttatacttttcatttctcataaacacaggtgtgtaaactgctagttttagcacctttgaattttatttgtctagaatggtttcaactttgccaatatcttttcttgcttattgaacagatgctttgaaaagagagatataatttaaaactagaaattaaaatagtgtgcagcacagaacaaaagtact

SEQ ID NO: 97 - DNA, 401 bp, Homo sapiens
ccagctgtcaaagtctagggcggtcggggaggacccacaaacgatcccggccctggggatacactgccagccggcctgaactcccggggagaggccgggggtgggcagcgaggtctggaaacgccctcctttctcagactgagaccagatcctctgcgaactgaggtctgagacgcgttcgcccccacaccaccgcgcttsgtttctttggccccgcgcgtgtccacccggaccctgcatggaacatgccctttgcaaatctaaacccgcgttggggatcccggataattccgggccttgcatgggcgcggctaacaagagtgcccaccagggaactcgcctcccagatcccggctccggatgccgcctgtccttcggcaaagaactgcaggtcatcatgc

SEQ ID NO: 98 - DNA, 401 bp, Homo sapiens
catcttagcaatattgagttatccatccatgagcatggtatacagatcatttatttaggttttctttaatttccctcagcaatgttttataattttcactgcataggtcttacataggttttcgaattctgttcctatttcatgttttgatgctgttataaatgatactgcttcttaatttcaatttccagaagtgtgttsctcgtatatagaactacaactgattttatgtatttactttgtaggcattaaccttgctaaattcatttattagttctagtgactttttttgtagattctttaggattttccccatttcacaatcataatacctgtaaataaggagctttatttcttccttcaccagcaacacgtatgcctttcgttgtttgcatttcctc

SEQ ID NO: 99 - DNA, 401 bp, Homo sapiens
ttgggaggctgaggcaggagaatagcgtgaacccgggagacggagcttgcagtgagccgagatcgcgccactgtactccagcctgggtgacagagggagactctgtctcaaaaaaataaaaaataaaaataaaaataaagaaaagaaaagaaaaaataaaagaaaatcaaagattataaatatagtaatatttgggagttyatttttttctctaaatcctgtcagagagaaagcagttctcatttacttaaatacacttcaagctgaatcactcatgaaatgtcattttatttaaaatatgtcaatgatacttgaataaacacatccacaaccactaaattcatcaaatgaaaacaatggactgggaaagttcatttctatcacaggaaaatttcaagcag

SEQ ID NO: 100 - DNA, 401 bp, Homo sapiens
tgaatcactcatgaaatgtcattttatttaaaatatgtcaatgatacttgaataaacacatccacaaccactaaattcatcaaatgaaaacaatggactgggaaagttcatttctatcacaggaaaatttcaagcagttatgaagcccagatatttcaatgatcttgggatgattgtagttaaatttctttgtgtggctgygcctaaacccagacgggagaaacaagtgcaaactggctgagttcattgctgtccatataaggtgtattccaaacacgtctttctatgccagcacattggcagaacattaaatctttgtatgcaaaacaaagaaggccattaaagtaattaattatatatagattatgtaattttttaaaatcccatttgccccaattatt

SEQ ID NO: 101 - DNA, 401 bp, Homo sapiens
gggttcttggcttcactcaaggatgagccagtggtagtgttaccagtggaggttgtccaggttcttggtgttttgaactaagaattggacaaaatccacaaagcaatgaaaaaaaaaagcacagatttattaaaacaaaagtacactccacagagtgggagtgggctcaagcaagtggcccaagagcccaggtacagaatyttgggggtttaaataccctctagaggtttcccattggttacttggtgtatgccctatgtaaatgaagtagtgacccacagttggttggcttggttgtgggaagcgaacaatcagaggctgaattgaagttacagcattatattcccatacaaatgaagacttggcccacgaccagtctgattggttgcaggaggggactc

SEQ ID NO: 102 - DNA, 401 bp, Homo sapiens
catacacacagatgcacacacatgcccactgatgcacacacttgcacacaaacaggcagagggaggtgtgtgctttgcgtggcaaggggctgggaatatcacagagcaagccattacggccgacccccagatcccaggacctgagagaatacagtttgtgatcacaaggcctctccaggagcacccatcataaagttcaaycaattaagcagacagtttctatggagacgtttactttactcagctctggagttctgcagctcctatgtcctgggagatacgctgtggctgcaagttgtggtcagtctgccgcaaacccaaagccccgtaatctctttgcattactctctgcagataactgagttgtcttagaaagaacaggcatgggaatgagatgacat

SEQ ID NO: 103 - DNA, 401 bp, Homo sapiens
cctaggtatgtatccgacagaaataaaaacatatatccatccaagagttatacatgaatgttcacagaggcattattcctaacagccaatacgtgaaagcaagtccaaatgtccatcaactgatgaataaatgcaatgtggcatattagtcagtgctatagctaatattccatctgtgcaatggaatattagttagctatraaaaggaacaaagacctaatacatgctatggccatggatgaacccctaagacataatattaagtgaaagaagtcagttaagaaaggccatctactatatggtcccatttatacgagctgtccagaaagacatgggttcaagtataaataattttcctaaaatataattcactcctctaccttatactgaagtttaattaa

SEQ ID NO: 104 - DNA, 401 bp, Homo sapiens
gatactgaacatacaaccacaggccatgagcatctggacaactttgtgacattatgagtagttcctccatgtttggactcaccgagctaggaggccatcaaatttcattattgtctgaagtcactttcagggctccagaaatatcctctagagagtttgagaaacaaaatgccacagttttcttattcttgttccacataytggtattgatacctcctaatgatgtaagtgcaacagaccccccaggttacaagtcacagtaacctgagcatgtgcagatgaacccagcatgcctgattagtgaccctggagcaggctggaaagaaaaattcaagcacatgtgaaatgtaagtaccgaacccagaaatggggaccaaattaagaagtgaggggtgccccct

SEQ ID NO: 105 - DNA, 401 bp, Homo sapiens
ttctctttctccatccctaactctgggcagggaaggtgaggtagaagacagcaaggaggattgtgaagtggagggggcaagactcaaattctgtaaaaattctctcctaagtcttgggttgggactacaactgctattgacctgtggccagatggtaacacctgtaaatttaacacagtgaaagtaggattctgtccaacrtcattgagagcacaagtgcctaccagaagcaagagcttgcatgagaaagtctcactcagctctgtctataacagtcctcaattaaatatgaaggcaaatgggtgacgtggggggaacccccagaaaacccacactttccacttcgttttcctcagattgaaaaataaaaggccagtcagtgataatctgctgaaaacaaa

SEQ ID NO: 106 - DNA, 401 bp, Homo sapiens
gacagctcttcttgttttttgtacagctccccgccaccgccccccaccccaaatgtgccatttcgagtggctggagtacaaggtacaagtagaagaaccactagaaacagcattggaaaagtttcctggtcatgaggtggagcgcttaggacaggcaagtaaaggagcatgttaatcactccagagcagagagctactgcrcacatcctgcaggacagacagggcctgcgggagctacactgaggaagcgtaaacgagaagtgacccccaaactgggctgcccatcagaacacctggcgaatttgttaaaaatagagattcacagcctcgttatggcagggttttgcttatgcacagctggtgtgggacccacagtgtgtatttaccgaagtgctatgggc

SEQ ID NO: 107 - DNA, 401 bp, Homo sapiens
gcgagcccatgctgtttattggtgctcaaacaaagaacccgtgaggatgtgggggttgaaaggaaacagtgtatcaagtgaatgagaaacatacagctgcttgagataatgggagtgctaaaagcaaggagccagcaagtctagcaaacatgcaagccctgccccagcttttctcccaacactcagctcttctcccaacasaagagcttttggaggtcacttctggggcctttgctaagcaggccactccttgcttctcctgcagacaacaaggtttccagcatgattattgcaatgaccaatggaacaatgatgaaacatccatttttggacactctcaaagaagtgagttcagttcatatttatcagcattttgcttcatgtattttggtaattaatac

SEQ ID NO: 108 - DNA, 401 bp, Homo sapiens
atgtatttctttaatggctgttttcagttctttagggtatatacttaggagtagaattgctggatcataggctaattctatgattaaatgtttgaggaaactccaaactgtctccatagtggctgcactattttacattcccagcagtagtgtgtgagggttccaattctccatatcctcaccaacactttttttcttttwttattattattattattattattattattattattattgccatcctagtggatatgaagtagttcctcattgtggttttatttttgagacagagtctaactctgttgcccaggctggagtgcagtagtgcgatcttggctcactgcaacctctgcctcccaggttcaagccattctcctgccccagcctcccgagtagct

SEQ ID NO: 109 - DNA, 401 bp, Homo sapiens
tcacagatctgattggtgcagtttcgtgctccccacaatgtacttaacttgccatttaaagtcagaatttgtctgctaaaaaaaaagaaaaagaaaaaaaaatgcaatccagcaccgtggctcatgcctgtaattccatagctctgggaggcagatgcgggaggattgcttgaggccagtctgtgcaacatagcaagaacytttctctcaaaaaaaaaaaacacaaaaaacaaaattagctgggtgtggtggtgcacacctatagtcctagttactggactgtgggaggcaggagcctagaagttccaggttatagtgagctatcagtgcaccactgcactctagcttgggcaacagagtgagaccctgactcttaaaaaaaaaaaattggctgggcacgg

SEQ ID NO: 110 - DNA, 401 bp, Homo sapiens
cctgagagacacagagagaaatcttctaaagttaaattctacttagccaagactagaatcgttagggaccacctgaaatgttccccaatcctctcatttcgtattgttcccagagagtgatggggggggtcttaatttatttgggctcacacacttctgagaatcggagataaattgtaggatcccgtcgagaaatgtgcrcctctgcacaataacaaacttgcgtatgatttcaggagattcacgggaagtcctgagccctctcttcctgttagaagtcctagccctgggactaatccccaattagctcaagggtgccggctctggaggagtgggtgccctgggggcggggtgggcgggggggaagccgggtgtcggttggggtggtgggtgcgccattt

SEQ ID NO: 111 - DNA, 401 bp, Homo sapiens
ggcctgtaatcccagcaatttggggagctgaggtggacagatcaattgaggtcaggagttcaagaccaccctggccaatatggtgaaaccgcctctactaaaaatacaaaaattagccaggtgtgcctgtagtcccagctactcaggaggctgaggtgggaggatcgcctgtgcccagggaggttgagactgcagtgagcygagattgcaccactgcactccagcctggacaacagagttagaccttgtcttgaaaaaaaaaaaaaagacagttgtcattttaggtcaaacggacaaattctttaaacaaaatataataaaacatcaaggggaaatagaaaatctgcatggtattattatcacagaagttacaaaaattaaaacagtagtgaaaaaaatat

SEQ ID NO: 112 - DNA, 401 bp, Homo sapiens
ctggacacagagacagacgggcatcaagggaagatgatgtgaggacacagggagaagacggccatgtacaagccaaggagaggctgacacagagcatccctcgcagcctcagagggagccagtcctgcccacacctgatctgggaccgtggcctccagaactgagttggccaatgtctgctattgaagctgccagccacartgctttgttgtggccgcccccacaagctcacacaggggtggagaagcagacgttgccacaggtgtcacgtaaatcaagacagtgctatgccaatgtcatgccaaggcagttgatatttgggccaggcgcagtggtgcacacctgtagtcctagcactttgggaggccgaggtgggtggtggattgcttgagcccaggagt

SEQ ID NO: 113 - DNA, 401 bp, Homo sapiens
ggaagtgagtaggggcctggggtctggggagcaggtgtctgtgtcccagaggaataacagctgggcattttccccaggataacctctaaggccagccttgggactgggggagagagggaaagttctggttcaggtcacatggggaggcagggttggggctggaccaccctccccatggctgcctgggtctccatctgtgtycctctatgtctctttgtgtcgctttcattatgtctcttggtaactggcttcggttgtgtctctccgtgtgactattttgttctctctctccctctcttctctgtcttcagtctccatatctccccctctctctgtccttctctggtccctctctagccagtgtgtctcaccctgtatctctctgccaggctctgtctctc

SEQ ID NO: 114 - DNA, 401 bp, Homo sapiens; identical to SEQ ID NO: 8

SEQ ID NO: 115 - DNA, 401 bp, Homo sapiens
ctcaacctagggcaagataagcttgacctgatgggtcatggctctgtacaatagtttagtcttaaacaactaagatgctacgtatctaagaacactcatgactcaaaggcttgcaaagggctctttaaatttgctcttttaatgcataaataaatccatatcatgtattactttctggaaaagggttaaactcaaatatcygaaatactttcattataccaggtcaagaaaaatgccacagccagaaaaatttattttaaaatagaaacatacattaagctttaaaacaaccaactctcaaacaaaagaggaaagagcctttgatcccagagtccatgcggaatgaattccatacgtgtttgaaattcacataaggagcacttagaaaaccacctgaaatg

SEQ ID NO: 116 - DNA, 401 bp, Homo sapiens
gataaaatgctgacaatcatttagtctcagggtagtcattacatatttactcacaaactgaagtaaaaattgggttcatcctaaacagccaacagtaggggaatggctggggaaatgtggttaacaaatggtagaatagtatgcagccgtcgaaagtcacattcatgaatgcttttaatagcatagcgaaggacaaaattkatttaagagaaaaatgaagaatacaaaatacattaccaatcaatttcaactgaacaaaaatctttttttcaaaggaaaaggtaccaaatggaaatggtatataccatttagctttagcaattgagaagtgggaatttgggtgatatttatttttctcattgtgcttttgggggaagatttttagccatttctacaacaaa

SEQ ID NO: 117 - DNA, 401 bp, Homo sapiens
tgagaaattgttgcccagtctcatagctggcacaaagtaagcccccaataaacagctgttgaaagacggaacatgccatttctagatcaacattcaaggtaggctctgataagcccaaagaaaaaaaatggaagatcatgaccttggttttaaactctagatttctattaacactgcttcagatcatgttacccttaattyagctacaacacacccctgactcatactgagtccactgagaccccaaacaagacaaccctcatcctatatttgtgggttcagttctgaacctaagagcagaattttatctacatctccaaaggcaaaatgtgtttctctcttaggggcccagcctgagctgtgatgactagttgtcttcaagagtacacagcaacataagc

SEQ ID NO: 118 - DNA, 401 bp, Homo sapiens
ggtaggctctgataagcccaaagaaaaaaaatggaagatcatgaccttggttttaaactctagatttctattaacactgcttcagatcatgttacccttaattcagctacaacacacccctgactcatactgagtccactgagaccccaaacaagacaaccctcatcctatatttgtgggttcagttctgaacctaagagyagaattttatctacatctccaaaggcaaaatgtgtttctctcttaggggcccagcctgagctgtgatgactagttgtcttcaagagtacacagcaacataagctcctgggagcaggaccttggcaagatggttttctactgtgttgccagcactggagaatggggcatgttacacagcagacgctcagtgaatattttct

SEQ ID NO: 119 - DNA, 401 bp, Homo sapiens
gcggagctcaggtggtaatgctcacctacctgccattcacctcctgctgtgccgcccagttcctcacagaccacggaccagtactggtctgtgactcaggggtgggggacccctggtctaaagctcaaggaggtggaacataacattccactccagtgtgcaccttacccggtggcttccttctcaggagcacagtatgtragcagggaaaggagtaacctgctttgcagcagagaggcctggcaaacactcctcagcaggcagttaatgtcaacatcaacagccatgagtcatgttgatagaatgtaccctagacacggtatgatgagaatagcaccttacctcccccaaacccataaccctaccctgagcatgaaaaaaaaaatcagaaaaaaaaaatt

SEQ ID NO: 120 - DNA, 401 bp, Homo sapiens
ttggctttgtcacttgtgaatggtgtggacttgggcaaggcatctcacctctttattctttagtttattctgtcaactgagagtggtagtgtgtacttcccaggtttttgaggaaaataaatgagatcatctttgtgaaaggcctccttaaagtttcaggtgtgtgcagatatgtatgataacttttcttcatgatatcaktgagcagaagatcatctctgagcactgtaattatagaattaggaattaggaaataagaatttcaacatttttgaaaatccctgggattatttaattgaataatcaaagaaaatagccagggcaaatgtgtcaactccagcccttggggagtgatgggaagatgatgggaaacagattgtaaccaaaaaaaaaaaaaaaaa

SEQ ID NO: 121 - DNA, 401 bp, Homo sapiens
tgtaattatagaattaggaattaggaaataagaatttcaacatttttgaaaatccctgggattatttaattgaataatcaaagaaaatagccagggcaaatgtgtcaactccagcccttggggagtgatgggaagatgatgggaaacagattgtaaccaaaaaaaaaaaaaaaaaaaaaaaaaaaagagaataaatctgcrtgaagtttaaatatgatgttgttttcagtcacgtaaaccaataaagcactttgaacccttatcttattttcatttccacatattataggtccatcagcttgtgatggagagcgaaacaccaggttaggagcagctacaggtatggggcactgggagggtgcttgccacagatgggggcggacctgaggcaggtgtgaggc

SEQ ID NO: 122 - DNA, 401 bp, Homo sapiens
ctaaaatttaaactcacaaatgtacttttattttgcccactttttccattccatcctattgaacttggacgagtcatcaataaatgccttcatttagaaagctgactttttaagttacaaatgatgttgccatattcaggatcagtaagaactgggtccgctccacgccccaacagtagtgggatttcccaccacccacayacatgggaaaagataagtctcctgcaaagtttttgtcttttaagctgaatttatttaactcaaagcatctacccaagagctaagatagcctatagaactccaagtgtggacagaaataattaacagtaattaagacacttgtcattgcttatgtttattaactttaaactattacccatgcttaagtgccaagtgaggat

SEQ ID NO: 123 - DNA, 401 bp, Homo sapiens
tgtaagcctcagttttcatctgtgatgatagtattacaaatgaagtaggtacaattatctttacccgtaagggaacttcccatgagcagtgatggctccaaggcatagaaagatgggatgggacttcactgttccctaacaagctataccctccaacccaaaagcacaatctaagtaaaagctatgagcttgctgttgaarccaattcaatggtacaatttgatgttgaaataaaattagaaactcctagccccttcctagcagaatgctacccctcagcccctcctttataagctgccagttcccaaaagaaaatccgtttgtgatccaagtcatctctctaaaatatttcttttcatgacaatgttttaccacaactctactctgatgctttcctctga

SEQ ID NO: 124 - DNA, 401 bp, Homo sapiens
agaagctgaaagtcccatgaagcccaattgagatggatggagacagagggagcatgagacaaacaaagtttaaaaatgaattaattgcaatcattcataaggtcaaatgccaactagacatcccaactccattgggtgtgtgtgaatactacaaagaacatagactccatccctaactttggaacagagcgaatcaactcygtttggagacctaagcagagaccacataggctggttagcgaacaatgaagtcctccattagaggaggattgcaggtgcaatagtcagagatggagcaatcactgtgagctggtgggggtggaaaacaaggcgttcttaaggagttggagcccaaaaagaccttggtgaataggtatttggatgcattcctttggagagca

SEQ ID NO: 125 - DNA, 401 bp, Homo sapiens
tggagttaggccgccataggccaagcaacactaaggcttgctggcaacaaactgaagcaaagaggtaggcatgaaacagacctccccgtgagccctgaagaaagaaccaactgggcttacaccttgatcttggccttctatcctcctgcactatgacagaatacatttctgttgttttaagccacccagtttgtggtactyctaatataaatcccaagtctggttttctgcctccatgagtttccagaattagaacctaagtgtaggaaacattcttctaacccagggctatttctgccacttcaggaagaaggcagaaaatgtgaccacttttgacctcagttctgaggagcacaagaatctaaatcagggaaacatcccttcatcagggtacaagttca

SEQ ID NO: 126 - DNA, 401 bp, Homo sapiens
ggcttacaccttgatcttggccttctatcctcctgcactatgacagaatacatttctgttgttttaagccacccagtttgtggtactcctaatataaatcccaagtctggttttctgcctccatgagtttccagaattagaacctaagtgtaggaaacattcttctaacccagggctatttctgccacttcaggaagaagrcagaaaatgtgaccacttttgacctcagttctgaggagcacaagaatctaaatcagggaaacatcccttcatcagggtacaagttcaaagggctcagttaagataaattagtatattcaagacgtgagtgtttccattgagggactgttgtgtagcagtgtacactctggaagccaagacacagtggtcatcctcacaag

SEQ ID NO: 127 - DNA, 401 bp, Homo sapiens
tgatccaagtcatctctctaaaatatttcttttcatgacaatgttttaccacaactctactctgatgctttcctctgaataaaatcagcaggttaatgattttaaatatctgggttaatgtttccatttgaaccattccaggagacaaacatgagaatgactaggtatcaaatgaatataggcactgagaaaaaaaaaatkttgagtgggcattttgctcaggattgggtactacagtagccgatttatttaaagcaaattttttgttattgttgttttattttatttatttatctttcttcaacttttaagttctaggacatgtgcaggacgtgcgggtttgttacaaaggtaaatatgtgccatggtggtttatagcaagtttttaaaatcctccccag

SEQ ID NO: 128 - DNA, 401 bp, Homo sapiens
ccccaagtccacatgactttgggcaagtcacttcaccagtctgaacctcagtttcttcatctgtaaaatggaggtggctatacttctcacatggagcggtcgtaagaattagatgaaggaatatatttagaggatttggcacaatacctcaccacagaaaaggttcaataaatgtgaaccgttattgtgtctgttcacatyaaatgaaaaaatctcacattgctcccaggcccttgggatgatccacactggctttcgccattggtgactctactggtgccacacagggcccacggggaggagggaagacactgtgacagacctcactgagcaggggacatctccttctcccccttggatttcccctcaagtaactgcatttagagagctgctgtgaagtc

SEQ ID NO: 129 - DNA, 401 bp, Homo sapiens
tagtgccatccttcagatgttaggccagcaccttcaactacaaattaggcaaagctaagagacatatatgattaatttcatatattaattttatatattcctatattaatttcccaaaaatattttaaacaaaggttatacaatgaagctaatcaaatagaagtggcaaatcaagataaggaaagaaaacccaagaattcrtgggagcaaaagggctgttgtaactgcatcagaatgtgggcatgaggttcctagtttccagagcagtaaaaacaaaacaaacagcaacagcaacaaaaacaaacccacataaaaccaccaagttacgcagttctcatcagaaagaggaaagcattccagattcttagcagagatgcattttccctcccaaatttctaaaa

SEQ ID NO: 130 - DNA, 401 bp, Homo sapiens
gcaggggggcatactgcatggattttacagggctgtttctgccctgagctttcttcctcatctgtaaagtgagggatttggatggaatgcttgctaagggatgtttaagcattgtgatgttgtaatatctataagaaagcaattaaactctgcttttataaaccttcacatttattgtggcatttttcatcatcagattgkcaaagattgaaacacatggggctaatgaggagataacaaaacagatgctcttggccttactggtgggtgaataaattggctcaacctctgtggatatctataaaaatataataacaatgacaatcataatttgcagcgcatatacccatttacactgataactcatttctgggaggttctcctagagctatgtttgcaca

SEQ ID NO: 131 - DNA, 401 bp, Homo sapiens
agccaggtgtggtggcacatgcctgtaatccaagctacccaggaggctgaggcaggataattgcttaaacccaggaggaagaggttgaggtgagctgagatcacaccattgcactccagcctgggcaacaagagtgaaactccgtcaaaaaaaaaaaaagcaagtcttattaacacaggagcaagaggtggttacagattytaggtacagagcctgggaggattctgggaaggtttgagagcaggggagacccatgatgctagtgagttggatctaaagcaacttctaaacatgcccctgctctttttcctcaggcagcaggtggttacattactcttgattggaactcctctcttttgccttctcagaatgtcttgtttttgtttccagaatctctgtga

SEQ ID NO: 132 - DNA, 401 bp, Homo sapiens
gaaacaacaggtgctggagaggatgtggagaaataggaacacttttacactgttggtgggactgtaaactagttcaaccattgtggaagtcagtgtggcgattcctcagggatctagaactagaaataccatttgacccagccatcccattactgggtatatacccaaaggattataaatcatgctgctataaagacacakgcacacgtatgtttattgcggcactattcacgatagcaaagacatggaaacttttcttgaagttgcatctgcacagcaaacatactgttctttttaagttagagattttgttactagcaagtaaaaatagagatggctttattcatgttttacataaaatagagaaaatatcagttttccatgttaaggaatactcttat

SEQ ID NO: 133 - DNA, 401 bp, Homo sapiens
ccattttgctttcccaactaattattttattatactctcttttgatgcctgataaaccccttcttgttctttttcagaattattcagtccactattgcttatttttcaatgttggattcagcttgtctagtaccaaaagtaccttgttggtcttttccagaaagatttatggatcaacttaagaacaattgacatctttakgattttggatattcctattcaagaacagaatatgccttttgatttaatcaagttatctttgtgttctttattaattttaataaaatatttattgaaatataattcaaataccataaaatatgtcatttattttgtttctttattttatttttttagagacagtctcactctgttgtccaggctggagagcagtggctcca

SEQ ID NO: 134 - DNA, 401 bp, Homo sapiens
ggactacagccacgacctcatgctgctccgcctgacagagcctgctgataccatcacagatgctgtgaaggtcgtggagttgcccaccgaggaacccgaagtggggagcacctgtttggcttccggctggggcagcatcgaaccagagaattgtatgtgggggcagactgtgtagcccaaggcggggatggggactcctgygtccaagggagaaagggccagggaagcaggtgaggtcgggctgcagccctttttctcccgggttcgtagtctcatttccagatgatctccagtgtgtggacctcaaaatcctgcctaatgatgagtgcaaaaaagcccacgtccagaaggtgacagacttcatgctgtgtgtcggacacctggaaggtggcaaagacacc

SEQ ID NO: 135 - DNA, 401 bp, Homo sapiens
tactatgttaggtgaaagaagccagacactaagggttacatattgaatgattcaatttacatgaaatatcaaaaattggtacatccatagagacagaaagatttgtggttgccagaactgggggcaggtgggaatggggagtggctgcttaatggctacggaatttcctttcgaagtgatgaaaattcttagaattaacgrtaatacatgcacaacattgtgaatgtacttaatgccattaaattgtactttttttttttttttgagatggagtctcactctgtcacccaggctggaatgcagtagcgtgatctcagctcactgcaatctctgcttcctggattcaagcgattgtcctgtctcagcctcctgagtagctgggactacaggcacgtgccacc

SEQ ID NO: 136 - DNA, 401 bp, Homo sapiens
attccattgaacggacttaacatatttaaatagccccctgggggaggacagttaggtggtttccagtccttggggatttcaaacgatgcttccggaaataaccttataaatgcagtttttcttatgtgataatatatctacagtgtacgtccctagatgtggaattgagcctccaaacttttatgtttataattgaggtgkcatttacatggcgtgccatgcccaggtctccagtgtacagtcagctgtatagtgtgttttatcaccccagaaaccccctcatacccttcccagtctctcctaccctaggagcagccactagtctgatttctagcgctgtagatcactttagtgtattctacaactttgtctaaatggtattgtacaatttttatggaggt

SEQ ID NO: 137 - DNA, 401 bp, Homo sapiens
aggaaacaggcatctaagtggggatgtgaagaaaacagggaaaatctttcagttgttttctcccagtggggtgttgtggacagcacttaaatcacacagaagtgatgtgtgaccttgtgtatgaagtatttccaactaaggaagctcacctgagccttagtgtccagagtttttattgggggtctgtaggataggcatggrgtactggaatagctgaccttaacttctcagacctgaggttcccaagagttcaagcagatacagcatggcctagagcctcagatgtacaaaaacaggcattcatcatgaatcgcactgttagcatgaatcatctggcacggcccaaggccccaggtataccaaggcacttgggccgaatgttccaagggattaaatgtcat

SEQ ID NO: 138 - DNA, 401 bp, Homo sapiens
ccaagtagttgtgactgtagccacgagccattatgcctagctaatttttttattttttgtagaaatgaccttttgccatgttgtccaggctggccttgaactcctgggctcaagcaatcctcccaccttggcataccaaagtactgggattacaagtgtgagtcagcacatccagctgctatgtcgtcttctgtctcctcrtgtttctggagagatattcgttgtctctgcccatatagtcccttttgtatccagtgactctttgctgcttcctcgagatcccgggacataggattcctttagaaaagtgagagccctggggcttttggtctcatctcctggatgcatccctcactcattcctctctgggatccttccatgtccaaaggggcctctggaaa

SEQ ID NO: 139 - DNA, 401 bp, Homo sapiens
gagaccttgtctcaaaaaacaaacctgctgggcgtggtggctcacacctgtaatcccggcactttgggaggccgaggcgggaggatcacgagatcaggagatcaagaccatcctggctaacacagtgaaaccccgtctctactgaaaatacaaaaaaattagccaggcatggtagtgggcgcctgtagtcccagctactcrggaggctgaggcaggagaatggcatgaacccgggaggcagagcttgcagtgagctgagatcgcgccactgtactctagcctgggggacaaagcgagattccgtctcaaaacaaacaaataaacaaacaaaacctatagctgtggttcttcaccttgcctacgcaggatgtgtataaagtgagtatttgtcttagctgtac

SEQ ID NO: 140 - DNA, 401 bp, Homo sapiens
ggctaacacagtgaaaccccgtctctactgaaaatacaaaaaaattagccaggcatggtagtgggcgcctgtagtcccagctactcgggaggctgaggcaggagaatggcatgaacccgggaggcagagcttgcagtgagctgagatcgcgccactgtactctagcctgggggacaaagcgagattccgtctcaaaacaarcaaataaacaaacaaaacctatagctgtggttcttcaccttgcctacgcaggatgtgtataaagtgagtatttgtcttagctgtacttatgtcagggaaaaggcatgtggatttaagcagcattttaaaaagctcgcttaacatctcaaacacctggagccactgggaaactgagagctgacagtgccggaaatcacaaa

SEQ ID NO: 141 - DNA, 401 bp, Homo sapiens
tggtgggcatcatgaagccagtgaaatttcctaagagtttatggacccaatattctttttcatttcttatttactgaagacactaatgaagcaatgattgctaagagggcgttctggtagcagtcactatcagggctataatagctacccttagtggagtgcttagtgtcaggatctgtttcaggcattgaaatacattawgtcttgtcatcctcagcacagaactgtaagccaagcatcactatccccatgatacagatggagaaacacactctcagtgagattaaggaactggctcacgcatataactagtgcaggcagagtggggtctttaccactcatgcagtgtggatatctgtccctccagatcgcatgttgaaatgtgacccccagtgttggag

SEQ ID NO: 142 - DNA, 401 bp, Homo sapiens
aaaaaactgaactgtgttgtgggcccagcacagcctcagtctacctggagggcaccctggagcaacggtgttcatcaagagtgtccacttcaggccgggcacggtggctcaggcctgtaatctcagcactttgggaggccaaggcgggcagatcacgaggtcaggagatcgagaccatcccggctgacacggtgaaacacyatctctactaaaaatacaaaaaattagccaggtgtggtggcgggcgcctgtagtcccagctgctctggaggctgaggcgggagaatggcgtgaacccgggaggcggagcttgcagtgagccaagatcgcgccactgcactccagcctggggggcagagcgagactctgtctcaaaaaaaaagagtgtcctgttcagactg

SEQ ID NO: 143 - DNA, 401 bp, Homo sapiens
gtcccatatcccttcagccctccctgttttatctgcagctgtggttgacagtttttgtgcaagctaacagcttctcctttttgccactggggtcagttaactttcagttacagacaaacctgtttaaaacacaaacaggcatatacctcttagaagagaacaaaaatgttgccttttcagatctcagactttaagaaattytggaaccaggaattgacaaacagtgcttggaattcaaggaatgaactgaccctaggcacttctaaaataaattccagctcagtagaaacaataatttgaacttgcatttcccatgagcaaactttcatgatcctgtagctataacacgactggatggtgagaatgtaaccactgctggtatcatcccaacttgtcagccc

SEQ ID NO: 144 - DNA, 401 bp, Homo sapiens
tttttgccactggggtcagttaactttcagttacagacaaacctgtttaaaacacaaacaggcatatacctcttagaagagaacaaaaatgttgccttttcagatctcagactttaagaaattttggaaccaggaattgacaaacagtgcttggaattcaaggaatgaactgaccctaggcacttctaaaataaattccarctcagtagaaacaataatttgaacttgcatttcccatgagcaaactttcatgatcctgtagctataacacgactggatggtgagaatgtaaccactgctggtatcatcccaacttgtcagcccacagtgttagcctttgggcttcctgggatatctgagtggcaagttacccatgaaatacattaggcaaacaggattcg

SEQ ID NO: 145 - DNA, 401 bp, Homo sapiens
tcatgttcacatgggctcaccttgccccccaggtcctacagggatgatcagagtggctcaggcagatgctgcgcacacagtggtttgcgtcaatgctggggaacctctgtttacatgtatttacaaatcttcaatcttataagggagatgcaaggaaatctgcttgactttgaaactcggagagatattatctttgttatstcggactgctgatccacctgccccggaaggacatgttgtatttactatcctagccaataaataaatcattatggaaggacattatctttattatctaagacagtgaacaaacatgctctgatgctctagagaaacatccataaaaagacaatctggaactcaaaagtggctaatacctttttcaggagacaggcagaaag

SEQ ID NO: 146 - DNA, 401 bp, Homo sapiens
taagggtaaaagcagggagagagtgagataggtcagtcccagtttctgcctgggcaatcctatctttcagacctggacaggcagataacagatagcagttcccagctttggaagaagagagaaatgtctccagagccgacttccagaagaaggtgggcagggattccaacagaggggtaggggctccagtcctggccggtrgggaagggacttcagggtccagctcctgtgcagaggggagggtccacgaggacagtccttgtagacaggtcggtgggacaagggccaggaccatctgcaggatgctgtccccgtggctcctcacagaggccaccttccaggactactctctgactagacacctcctctccagggcactagggagccatggaggaccatcg

SEQ ID NO: 147 - DNA, 401 bp, Homo sapiens
cagcctctgcccttcaggggccaaagagccttaagccacaaatatacccagaactctacccctcaggattccagcaccttcttcctgaggatatgagattcttaggccattcccacatcagtacctcgggagctgggaccttaccagtctcctccctcattgacctaagagttcggaactgacactttccctcccccagtwccttcacatccagcctcttcctcctttgaaattcaagagggtggacccactcctcactcaaacccagaagttctgatccccagccatgccccttcgggatcctgagcgctgccttattctgggtttggcagtggagtgctgccagacacagtcgatcgggacctagaaccttggttaggcataaataagcaggatgtgac

SEQ ID NO: 148 - DNA, 401 bp, Homo sapiens
tgtgttagataacaaaggttatctctcatattttctagcctctctaaccctctctgtttgtccatctgtttctgtatctcatcccatctatatagctattcattaatttctcatttaactgatatttgctagttgtcacaactgtgacttgatgaagataacataaactcataataatatccctcaataagaaaaggaacrataaactagtgtcattagcttataaaccgtgttagattaattggtggatcagaacacttgaaatgtcctgaaatctttcagctgacatttgaaagaaacttactattatttctccccaaattgggcaagaatcctagacaaatcatatgaaatttaaataaataaaagctttgaagctgaaagaaatgtttctaaaggat

SEQ ID NO: 149 - DNA, 401 bp, Homo sapiens
cccagccacaactgccagctctctgattccccaaatctgcatccttttcaaaacctaaaaacaaaaagaaaaacaaataaaacaaaaccaactcagaccagaactgttttctcaacctgggacttcctaaactttccaaaaccttcctcttccagcaactgaacctcgccataaggcacttatccctggttcctagcaccscttatcccctcagaatccacaacttgtaccaagtttcccttctcccagtccaagaccccaaatcaccacaaaggacccaatccccagactcaagatatggtctgggcgctgtcttgtgtctcctaccctgatccctgggttcaactctgctcccagagcatgaagcctctccaccagcaccagccaccaacctgcaaacc

SEQ ID NO: 150 - DNA, 401 bp, Homo sapiens
acagggtcaagcagctcctgttgcccaagcagccacagccgcctggccttcactgcatccagctgtgcagtggcagagaggatgctgcgtggggacagcaggaggagaggccttcaagacccaggccagctaagcctggggagatagaagcagccaccacgctgggggagtgtggacgcaggagtttgaggagttctgcgrggtgctgaatggggtcctcctctggcgctttcttgagggagttctgcaatgatccacattctttccagggactttctttcaagtgctgtcatatgctgccgcggcttgccggcagcccagcctcaggcggtcggagtcggaaggcgacagatgcagggctccctttcctcatcggttctctcaacagggcagggggacgg

SEQ ID NO: 151 - DNA, 401 bp, Homo sapiens
agagtggctggagagcagggcgtgatgcctcttgggccaccctgcaaagggctcacgagcctcctgaggaggatgcatttgatcagggtaacaggagtcagcagagggctttccaaggacaggtttgccaagaagcgagaacagactgaggggggaacgatagaggcactgggtgtagtcagacaccactgcaatggtagscgaaaaccgcgccgtgagaagactctgtggggagggtaactaggtgcatgagagtgagaggagcgaaaaaagcctcggatggctccgaagctcctggcccagcttccaggggttggtggcccaggaccgcccctgcacgcggcctcgggtgcctgctccaggcagacctgcttctctggcccattctcggcaccaatcac

SEQ ID NO: 152 - DNA, 401 bp, Homo sapiens
actctgtggggagggtaactaggtgcatgagagtgagaggagcgaaaaaagcctcggatggctccgaagctcctggcccagcttccaggggttggtggcccaggaccgcccctgcacgcggcctcgggtgcctgctccaggcagacctgcttctctggcccattctcggcaccaatcactgtcccatccgcccgtcagccragtcctctgagttcttcccggagcccagcaccctgatgcaattgtccctgctctgaaaaccctcagcaatctattgggtcttcagtagcatctactctttctagaaatgtcgaacaaggtttttcgattgtgctgcacgtgtggcagacacagaaacggaaacgaccttccagagaactggccaaaggacgggagctgct

SEQ ID NO: 153 - DNA, 401 bp, Homo sapiens
ctgcgcctcccagactggctcctctgagcctgaacctggctcgtggcccccgatgcaggttcctggcgtccggctgcacgctgacctccatttccaggcgctccccgtctcctgtcatctgccggggcctgccggtgtgttcttctgtttctgtgctcctttccacgtccagctgcgtgtgtctctgcccgctagggtctsggggtttttataggcataggacgggggcgtggtgggccagggcgctcttgggaaatgcaacatttgggtgtgaaagtaggagtgcctgtcctcacctaggtccacgggcacaggcctggggatggagcccccgccagggacccgcccttctctgcccagcacttttctgcccccctccctctggaacacagagtggcagt

SEQ ID NO: 154 - DNA, 401 bp, Homo sapiens
cttaagacaccactaaaaaactattagagctgaaatttggtacagcaggatacaaaatcaatgtacaaaaatcagtagtatttctatattccaacagcaaacaatctgaaaaagaaaccaaaaaagcagctacaaataaaattaaacagctaggaattaaccaaagaagtgaaagatctctacaatgaaaactataaaatrttgataaaagaaattgaagagggcacaaaaaaagaaaagatattccatgttcatagattggaagaataaatactgttaaaatgtccatactacccaaagcaatttacaaattcaatgcaatccctattaaaatactaatgacgttcttcacagaaatagaagaaacaattctaagatttgtacagaaccacaaaagaccc

SEQ ID NO: 155 - DNA, 401 bp, Homo sapiens
ttttatcttaatattttcttaaatttcatcaaataacattcaggagtgcagaaatccaaaggcgtaaaacaggaactgagctatgtttgccaaggtccaaggacttaataaccatgttcagagggatttttcgccctaagtactttttattggttttcataaggtggcttagggtgcaagggaaagtacacgaggagaggmctgggcggcagggctatgagcacggcaaggccaccggggagagagtccccggcctgggaggctgaggctgacagcaggaccactgaccgtcctccctgggagctgccacattgggcaacgcgaaggcggccacgctgcgtgtgactcaggaccccataccggcttcctgggcccacccacactaacccaggaagtcacgg

SEQ ID NO: 156 - DNA, 401 bp, Homo sapiens
gtccagctgcgtgtgtctctgcccgctagggtctgggggtttttataggcataggacgggggcgtggtgggccagggcgctcttgggaaatgcaacatttgggtgtgaaagtaggagtgcctgtcctcacctaggtccacgggcacaggcctggggatggagcccccgccagggacccgcccttctctgcccagcactttyctgcccccctccctctggaacacagagtggcagtttccacaagcactaagcatcctcttcccaaaagacccagcattggcacccctggacatttgccccacagccctgggaattcacgtgactacgcacatcatgtacacactcccgtccacgaccgacccccgctgttttattttaatagctacaaagcagggaaatcc

SEQ ID NO: 157 - DNA, 401 bp, Homo sapiens
aaataactaaaagagtataattgggttgtttgtaacacaaagaaaggataaatgcttgaaggtgacagataccccatttaccctgatgtgattattacacattgtatgcctgtatcaaaatatctcatgtatgctatagatataaaccctactatattaaaaattaaaattttaatggccaggcacggtggctcatgtccrtaatcccagcactttgggaggccgaggcggtggatcacctgaggtcaggagtttgaaaccagtctggccaccatgatgaaaccctgtctctactaaagatacaaaaattagccaggcgtggtggcacatacctgtagtcccaactactcaggaggctgagacaggagaattgcttgaacctgggaggcggaggttgcagt

SEQ ID NO: 158 - DNA, 401 bp, Homo sapiens
ttactcaggagttaccctcctttgatattttctgtaattcttcgtagactggggatacaccgtctcttgacatattcacagtttctgtgaccacctgttatcccatgggacccactgcaggggcagctgggaggctgcaggcttcaggtcccagtggggttgccatctgccagtagaaacctgatgtagaatcagggcgcragtgtggacactgtcctgaatctcaatgtctcagtgtgtgctgaaacatgtagaaattaaagtccatccctcctactctactgggattgagccccttccctatccccccccaggggcagaggagttcctctcactcctgtggaggaaggaatgatactttgttatttttcactgctggtactgaatccactgtttcattt

SEQ ID NO: 159 - DNA, 401 bp, Homo sapiens
aatctgaattttttcttcaaaagcgcctgtccccacaatgggtttgtactggggaaaaaacccacacacgggatgaggtgcccggcttctgtgagggttctgagcacttcgtttttcttccctttagattctgacaactgtggactaaaagggttgcaggcccagtgccggaattccagtatcaccttccaccaggctccycacacgtcgacatggcccccacttggtggtctggcaggcgtcggcggttggggacggcgccccggggaccccgcggccctagccttccccggcgccccgcgaacgccctccgcggccccagcagctgagcgcctgggtccctgagaggcaccccaggagggcggcgcgggcgggtaacgtcacacacgcgccgcatcctg

SEQ ID NO: 160 - DNA, 401 bp, Homo sapiens
caccacattaccaatcaaagaaagccccagatagcccagggtgtgccacagctgttacttggggctttcctcatgttatgtccaagccagcttcagctccccttcaattgcatcccctaacaatcctgaattgcatttctgaatctaggactagtttttaaggccctccaacgtggatacatggtttctttgccattgacmattgacaaaaagctttgaattcaactctatttttccctcatttggtcacctcaagtagtaaattgaatcactttaaactactggttttgtaggtcaaaaacaaatatttcataactttcagtgtaaaaaaaaattactcttgagcgactccaacacctagcaaccccatagctggcgctagcccacttggccagcactgt

SEQ ID NO: 161 - DNA, 401 bp, Homo sapiens
acaaaaagctttgaattcaactctatttttccctcatttggtcacctcaagtagtaaattgaatcactttaaactactggttttgtaggtcaaaaacaaatatttcataactttcagtgtaaaaaaaaattactcttgagcgactccaacacctagcaaccccatagctggcgctagcccacttggccagcactgtggttyggctggctcccagttttgtacaaccccacttccattttgctttcccaactaattattttattatactctcttttgatgcctgataaaccccttcttgttctttttcagaattattcagtccactattgcttatttttcaatgttggattcagcttgtctagtaccaaaagtaccttgttggtcttttccagaaagattta

SEQ ID NO: 162 - DNA, 401 bp, Homo sapiens
gtgttctttattaattttaataaaatatttattgaaatataattcaaataccataaaatatgtcatttattttgtttctttattttatttttttagagacagtctcactctgttgtccaggctggagagcagtggctccatcatggctcactgtaatatggaactcctgggctcaagtgaccctcctgcctcagcctcccmagtagctgggactacaccacacccaactaattttgtagttttctgtagagatgggatctcgctatattgtcccagcacacaccaccacacccaactaattttgtagttttctgtagggatgggatcttgctatattgtcctggctggtcttgaattcctggtttcaagtgatcctcctgccttggcctcccaaaatgctg

SEQ ID NO: 163 - DNA, 401 bp, Homo sapiens
tttgtatatcttcactggagaaatgtttattcaatcaattgcccatttttaaattgggttatttgtttttgttgtagttgagttgtaagagttccttatgtattctggacattaacccttcctcagatatgtgattcacaagtttttttttttccattccataggttgccttttcactctgctcattgtttcctttgacakagagacgtttttaagtttgatgtagtcccatttatctatttttgattttgttgcctgtacttttagtatcacattcaataaatcattgtcaaatctcatcttatgaagtttccccctaggttttcctctaagagttttatagttttacatcttagcatttagcctttaatccatttcgagttattttttttttttttttt

SEQ ID NO: 164 - DNA, 401 bp, Homo sapiens
caagaatcgcttgaaactgggaggcagaggatacagtgagccgagatcacgccattgcactccagcctgggtgacaagagcgaaactccgtctcaaaataaataaataaataaaatctaaaatttcgaaggtaaatttcgcatgtggaactttacttttcagatactgattgataatgcgtgatactgattgatgtaatgsgtaacaattttccagctagacagaagtaaattttccatagcttggctgggcatggtggctcacaccttaatcttagcaatttgggaagccaagacgggaggattgcttgagcctaggagttttagaccagcctgggcaaccatagcaagacctcatgtcaactttttaaaataaaagaataaattttttaaaaaaatata

SEQ ID NO: 165 - DNA, 401 bp, Homo sapiens
gtcaaggactgggaggccagagtgttggcaatgggaggttatttgtgtgacactgaagtcccccaggagatggcagatctgaaaggagatggaaactgtggtgttgccagagtctttgtgaatgagggacattccggttctctactgctgtgtaacagaccacctacatttagagacacagaacaggcccattttattacrctcacaggttctgaggatgaggcactcagggcgagttaggagggctcctcatgtctggggccccatgacttgaaggattgggcactggaattatctggaggctgctccactcacaagtgtggctcctgggcaggagggcccaaaagctgggctcagctgggactgtgaatggagaccctgcatgtagcctcctcgtatga

SEQ ID NO: 166 - DNA, 401 bp, Homo sapiens
aataaccagtatatataaggagctcaaactactctataagaaaaacacctaataagctgattttcaaaaataagcaaaagatctgggtagacatttctcaaaataagtcatacaaatggcaaacaggcatctgaaaatgtgctcaacaccactgatcatcagagaaatgcaaatcaaaactactatgagagatcatctcaycccagttaaaatggcttttattcaaaagacaggcaataacaaatgccagtgaggatgtggataaaaggaaacccttggacactgttggtgggaatggaaattgctaccactatggagaacagtttgaaagttcctcaaaaaactaaaaataaagctaccatacagcaatcccattgctaggtatatactccaaaaaaggg

SEQ ID NO: 167 - DNA, 401 bp, Homo sapiens
atattagggttggagaaactgttcttgggaaaaatgatctttttgctatgctcctggtgccagatgaaagaaaggaagaaagaaaaagtgagtggcagagagtaggcaaaggtgacatgattctctattaaaaaaaaaaaaaaaaaaagaaaaaaagtttccagtaaagagaaatgttttcctaggcaaaaagttgtaagraggaatagcttccatgtacgcaagcaaggccttcaatgatgaaagcaaagcttcctgagctgattactaactgcttctctggctggagatattcacagactgaggtaaccatgaatacttctgggaagggttcagagtggtccctctctgggttttcaaacaaacaaacaaaaacagcaacagcaacaacaacaacaaca

SEQ ID NO: 168 - DNA, 401 bp, Homo sapiens
ctttgtaagttacccagcctcaggtattcctttatagcaatgcaaaatgggctaagacacccattgagttctgctgcccagaatggagaagtatgactggcactgctgcacatattcattcaatgagaaaatatctcgattacaccaagcatctagttgtagaaatgagaagaggtgcctgctgaacattctggaaactcrctccttttttgtgctgtgagttcagactgaacaggatcagcaagcataactgtatctgtgttccatgccttatcctccagcaaataccaggaacattctggagttgaagctgtttccactaatagccagagaacagtccagttctaacagaggtgccagggatgctctctcctggagagcactgaatttcctggagacaa

SEQ ID NO: 169 - DNA, 401 bp, Homo sapiens
cagatctggtgttgaactggcctgactgtcccatagaactgatgttcatggtttctctgaataacacagaaatggagccttgttgtcttgaaacttgagaaagtgacatttgtcttatttgagttcctttcttaggaaactgaccatcaggcctcccgggtagtgtgaaggaagagagactcaccagatcacgccatctgracagtgagatgccaaacccctcacccatcatgagtgcctgaccagccacctgcctcctcctgaccaactcctcttcctcactctccctaattcctgttttccttacacacagtcacacttcttccctgctatatagtcagtcaggaagatggatttgaagctgacctcccatctcctggcctgtagcttctgatgcacgc

SEQ ID NO: 170 - DNA, 401 bp, Homo sapiens
cagggtctcgctttgtcacccgggctggggtgcaatggtgtgatcatagctcactgtagcctcaaactgctgggcttaagaaagccttctgtcttagccttccaagtacctgggcccaccaaagcactgggattacaggtataagccatcacacccaggccttctctcaggtttgtttttttttttttttgattttgaaamatgctggctgggtgcggtggctcacgcctgtaatcccagcactttgggaggccaagacggacagatcacctgaggtcaggagttcgagaccagcctggccaatatggtgaaaccttgtctactaaaaatacaaaaattagccaggtgcggtggcacgtgcctgtaatcccagctactcaggaggctgaggcaggagaatt

SEQ ID NO: 171 - DNA, 401 bp, Homo sapiens
gtccaggctggccttgaactcctgggctcaagcaatcctcccaccttggcataccaaagtactgggattacaagtgtgagtcagcacatccagctgctatgtcgtcttctgtctcctcgtgtttctggagagatattcgttgtctctgcccatatagtcccttttgtatccagtgactctttgctgcttcctcgagatccygggacataggattcctttagaaaagtgagagccctggggcttttggtctcatctcctggatgcatccctcactcattcctctctgggatccttccatgtccaaaggggcctctggaaagagaggctaagatccccctttgctgagaattccagctttccctctgctctccttgggattcacaatcctgcttcctttcagc

SEQ ID NO: 172 - DNA, 401 bp, Homo sapiens
tttgtaaatttttctgaggcagagtctccctctgtcgcccaggctggaatgcagtggcatcatctcacctcacgggttcaaacgattctcctccctcagcctcctgagtagctgggattacaggcgcgtgccaccacatccagctaatttttgtatttttagtagagacggggtttcaccatgttggccaggctagtctcsaactcctgactgcaggtgatccacctgcctcagcctcccaaagtgctgggattacaggcatgagccactgcgcccggccttgttttgtaaattatctcaccttccactcaacacaaaggtgggtgtcagaattcccttgctttatccttggcctgtattcatgatcacttaagtgacttccagtttttatttgcgcatac

SEQ ID NO: 173 - DNA, 401 bp, Homo sapiens
caaggctgtcagtcctgactgtgtggcatccagcacgagctgaggaaaggcccggcgagctgctacaactgtaggcctcggaccaggtgcctggctcttcacccgcacaccagggcccgacgtccatactacagccccatggaaaaacctcgcattccacctgtttacggttacatgagttcttcttcctctttaaaagtmtcttttttgagacaaggtctcgctgtcaccaggctgaaagtgtaggggtgcaatcacagctcactgcatcctcaacctcctgagctcaagtgacctcccgcctcagtctcccgagtagctgggactacaggtgggcgtgccaccatgcctggctaactttgaaaactgttgtagagatggggttttgtcacattgcctag

SEQ ID NO: 174 - DNA, 401 bp, Homo sapiens
cttaaacgacattcatcacctgtcaccttccaggctgaatgtttacaggccacgcataggcctccagttaggatattctaccacttcaagacattcggtaagactagttctctaaaataaacatgttatgtacagaaagctaacttttaaacaatctgaacatgagtaatgttttccctttcactggcattttgtcctacrcccatacctttcaaatttggactccacatcaaagtcttccacattcaagaccaggtcgatgttgttctcagcacccaggtgggacctcgtcgtggtgtacacgctcagctggaaaggagggggcgtcgagagtcagctcggccaggccctggcaggacgcactcaaatcccacggccagctggagggcggaagacctggc

SEQ ID NO: 175 - DNA, 401 bp, Homo sapiens
cgcccaggttcaagcaattctcctgcttcagctttcggagtagttgggattacaggcgcgtgccaccatgctcagctgattttattattttttagtagagacggggtatcaccatgttggccaggctggtctcaaactcctggcctcaagtgatccacccatctcggcctcccaaagtgctgggattacaggcgtgagccmctaggctaatcctgaatgtacattttctactgtcacagcagatattcccagcatccccctccaacttcccacccctgaacccagccccaccatgagtcccagcctgcagaagaccccacaccagcctcagtcccaacccaagacagcctccagcccctgagtgtcccctccagtgtgccccctggaacccgcagacctgc

SEQ ID NO: 176 - DNA, 401 bp, Homo sapiens
tgtagggaccccaagtcacccccaaactcttcagtccagatgctcttctttacctctcagacctggttccctaaagctgcatcctttcctgaacctcaaaagcccaacccaggtcctgtttccctagcctaggagccccttagccctccagattcttcctcagtgagcctctgatcctgtcccacccttggcgccttgatscctggtttccagaatccctttccactcccctaacccaccacctcccatcccccagccccagaccccaaatgacctgacgcccagatgcagtgtctgctccagacactgtctcctgtctcctaccctgatccctggaggctgctctactgtcagagcacaaaatctccccaccaggtccagccaccactccaaaagcccag

SEQ ID NO: 177 - DNA, 401 bp, Homo sapiens
ccaggctgtcaggcggcgcgcagggaggaggtgcagacgcggccgccaccgccaagcgcccagcgcgcggctctgaacttggcgccagagccctgaggacaccaggccccgccttcctggccgcacacgccttccgggcagggcttgtgggtgggcggccccggcgcgggagggtgccggggccggagaacccgcaacccrgtagaaagcctgtgatcagctgggcagcccagtcctccccgggctcagctcggcagacagccctctgatggctcggactgggccgggcgccggcgcctgcgggagggcgagggcgtcctttgaaggcggctccagctctcgccgccgccctctggctctaggctcccctacctctcgctcctgggccctgggggccgcgg

SEQ ID NO: 178 - DNA, 401 bp, Homo sapiens
tccacctcgagtgagggctggaaaacgaggctaggacttgctaggctccactcccagagagtcgggcattcccagcctctagacgtttagagataaaggaacaaataaataatgtttactaaacagacccagacttgggagtccagataccccgatatccagagaacaaaggcattcctaattttgccttaaagataaatratattgattcttgcaaaatatactaattaagaaaattaattctttatcatcaacgctcgtagcagagcacatctccccgtatacaccaggattgtaccccaggtgggcgcctttctcctcttagtttcgggaatgccctgctctgtctgtggagcagccgtcctttcgccactttactctcttaataaactcacttccac

SEQ ID NO: 179 - DNA, 401 bp, Homo sapiens
tgctgctccgtggccacgtgagccgacggatgcagagaccgacggcgggctcttcgttaccctggcccgcccaggctcccacggcccccagcccttctgttctcgtgctctgatggtggcgccgtgtggcctgccctgcgcgctaggcactgctcagctgctctgtgggtgggtcgtgggcacttgctgacactctacagrgaggggccttcctggagtcatgagacaggacactgagagcagccgccttgtctggagactgagtcccgtgtcagggacaagcagtgactgcctccctgacctctgcttcctccacgctggcaacaaaggcgggaagcaggaggccggcctgacaccaagaagcagatggcaggggccatgtttcatcccaatgcccagga

SEQ ID NO: 180 - DNA, 401 bp, Homo sapiens
agaaaaataccagtgaacttgaatacaagcattagaaagtattcaaacctaagcacggagacgggagaaaagtttcaacataaacagaacctcactgacattgaaacagcatcaagcagctcatatacatataactggagtcccaaaaggaagaggcaggcagagaaatatttgaagaaaccttcactgcaacattttcaracttgaatctcacagattgataagctcagtaagccccaagcaggacagacaaggcaaaagcgccagacaaatacacacaaaacacaaagaaacacagaagaacagctgccaatctgtcatcagaagcaatgcaagtcagaagacagtggactgaacgatgtgatttaacatgacagaagaaaaagtgcccatcaacctag

SEQ ID NO: 181 - DNA, 401 bp, Homo sapiens
cacggcacccccacctgcaggtacgcccctggcctgcccaatggctctgtgcctccaataagggcctaagaaaaagtcccacaggctgcccctggcaatatacaaaaatcagtagcatttctatacacaaacaaaaacctagctgaaaaagagatcaagaaggcaatcctatttccaatagcaacaaataatataaaatasctagggataaatttaacaaaagaggtgaaagacctctacaaggaaaactacaaaaaactgacgcaagaaactgaagaggatatgaacaaatgagaagacatctcatgctcaaggattggaagaactaatattgttaaaacgactgtgctacccaaggtaatctacacatttaaggcaatgcctatcaaaataccaatggc

SEQ ID NO: 182 - DNA, 401 bp, Homo sapiens
ggtgtgtgcctgtagtttcagttacttgggaggctgaggtgggaggattgcccgagctcaggagtttgaggctgcagtgagctatgagccactgccctccagcctggatgacggtgcgagaccctgtctctaaaaaaaaatttctactctagtgcttacagcagaccaattcatagtgactgaaaatggggacatcctacygcctatcgactgcccatcagcatctgggtggctagcccagctgtggtgcattcacaccaaggaatgccacttggcaattaaaaggaactactgttatgtggaacaatgatgagtttcacaaacactgtgccaagtaaaagaagccagatacaaaagactgagtgattccatccatgtaaatttctagaacagatgcatcc

SEQ ID NO: 183 - DNA, 401 bp, Homo sapiens
acagtggttttgagataattctccttggctacaaacatcaataacaaaggtcattccagtctgaggttggacaggcaattgctgggcagttgtccttgcagaaatattttttgtgtaagggtgtgatggcctctgtgccgggctgtggtttttgtagtcctttctggtatcaggcatgcaagcataagtgtcctctcttcrtggccttccccaccaactctttgtcagggctttcttaacattagtgactccattttgattctgacaactttcacagcatgtatttagagcagccgctaggtgttcatccacagaggtggccgtgagcagttcctgggatcataacaagagtccattctgcttggggcttaccacaaatgaagtaaagacaggcccttggg

SEQ ID NO: 184 - DNA, 401 bp, Homo sapiens
ctttgggaaaccctcaacagatgcccacattcaactggcaagattctcaaacaccggcccacaagccagcacctggagggctgctcatgcacatggtggcacttatcaataacttttcagtgatggaaaaatcttatcaataacttttcgatgatggaaagatgttcttgggtgggctaggaagtcctgtaagagctgtgwccttgcaagaggagagccagcagccaggcacggtgtgacaccttgggcctagatgtccctgcccctgacctgctagaaagctgactggtctgccacagaacgcttctgctttgcctccaggaaaaggccttgttatctgcgaactcccagaggcacccatcgcaggatacccagctttgcctgagggctcttgtccctgc

SEQ ID NO: 185 - DNA, 401 bp, Homo sapiens
cagctgttaggtagggcagtcagggacccctgaggccaggcagcccaggcaggaggcctgccaagatctgggaccagtgttcctggccaagggtgcctgccgtggtttaaggggcccaagtgagtgaggggtcctcctgaccttgcaggggtggaggttgtcacagtggggtggggagcggcggtctggacaggggcgagyggttgatgggtgtgaggacgaggagtgggtgtgtcctgttggttaggagtgaggagcatttggctccagtatcagacccgaacaagttgtttttctcgcatggaaaagacgcccaagcaggtggccctggctgcctgggggccgtgccgtgttctgcgttgttgtctcctaaccctaatgcctttcctggcgtcctgggt

SEQ ID NO: 186 - DNA, 401 bp, Homo sapiens
gaagagacctacagcggctctgaaaacatcacaagaatattaaaaagacacagattttcttaatatagactaaacaaggatggtagggacttccctccccaaacaaagctgctcagtaatttattgagaaccgataatgcaagtccctataaagtttttaatctgcacaaacagaatcatgttaatgtgttcacttgtccsgattttttttttgaaagattttgtttttgtttccacttgacacagtgaaggaaaaatatatatataaatccgcactgagggagatgtctttgaacacctccccgcctggtgggcagagacccagaccagccttgctggaagttgctctggacataaatgttggaactcctacccccagtagctcaatgcaaccgacgttt

SEQ ID NO: 187 - DNA, 401 bp, Homo sapiens
tgcttgttggcttccgtgtgtcatcagggttggtgggggtcagtcacttgaaatctttgcagcaatgagttgggaaaaactgaccgagtacctgctccatcatgcttgattgagtgctcctgtgtgctgggccctggcctggattagaatgcctctgctgtgggctttgggtttgtcaggatggaatcctacagcctcctygggggaaagcacacctcatgctgagcgctgtgttaggcacaggctgggatggagcacttcttctggtccttcattcttctggcaaagctggcttcctagaatggatgtgctcactggggtctgtgtggtgcccatgaggacaaagtgtagcagcctcctgcctgctccaagaaaagggcttgcgaaaggaaagccaaaca

SEQ ID NO: 188 - DNA, 401 bp, Homo sapiens
aagcggttgcacaagtggcctcagtcaatatacacaacccccaaaaaaaggttatcagtggtatcctctttttgcagatgaggaaactgaggctcccagaagggaagcgttatgctcctccccagaagctttaggtctgtgtgtggtggaagcaaagaagcccctttccctgttcttctacaggtcaggtcctagcttgcmggtttgggcgcgcttccgccgccctcccctcctgcccgtcaccaggggggaagcaggtgaaactccaatagtgctgcaaggcctatggcttcctacctggcccaggccagagtgggaaaaaaaacatctttccctcagtagctctgtcgacaccttctgtactaggagtgcaagacttctgccactgctgcagtgacctc

SEQ ID NO: 189 - DNA, 401 bp, Homo sapiens
gctggagggaggttttgctgactgagagggcagatgtcagccctggagggaagcgggaggcccagggtggggcctgagcctctgcatttggaaaattccgaggcccggggtggggcctgaacctctgcctttagaaaattccacaaagccttttcctcagccgtcagccttcaacctgaaggccaagctgagaggtccacrcaatgcagtcttcagaggccctggtggctggtttaaggggtgcggccaatgctgaggtgagtgagctccccagtgacattgtgccctgcaggtccacactgctggctggacacttcgaggctagcgccaagtatggattagccatgctacggtctctgttctgctcaatagcaatgactgtgaataggtaaataaatcgc

SEQ ID NO: 190 - DNA, 401 bp, Homo sapiens
tatgtttctattttaaaataaatttttctggctgtggcatttttcttgacctggtataatgaaagtatttcggatatttgagtttaacccttttccagaaagtaatacatgatatggatttatttatgcattaaaagagcaaatttaaagagccctttgcaagcctttgagtcatgagtgttcttagatacgtagcatctyagttgtttaagactaaactattgtacagagccatgacccatcaggtcaagcttatcttgccctaggttgaggcagagagagagaaatggaatagaaagatgggtggagaactgctggactcggtgtgtagtgaagtttaatgtttgttgaagttagtttttgtggggtttgttgttctcagctcacagcagcctccacct

SEQ ID NO: 191 - DNA, 401 bp, Homo sapiens
gcccaggaaagaattcaaaggcaagctggtggcgttagacagcacttttattgaagcagcagtgttatagcaacagaggtattgcttcttgtggagcagggctgccccatagacagcatgcccagagtagcagctcaggagcagttctgcagtcatacttatatccatttttaattacatgtaaattaagagacagagcaygtagaaatctttagaaaaaggacagtaacttccaggtcatcaggttattgtcttggaaaaggaagtgatttccaagtgttgccatggcaatgataaaatgacacggcacaatagtaggcgtgtcttatggaaaggtgctttcacctcttccctgccttctacctcaataatttctcatacatttgcaaaccagcctttcc

SEQ ID NO: 192 - DNA, 401 bp, Homo sapiens
gcttacttaaatggttgtttctgctaaggactcagaattccacttatgggtaatctcagaaaaaaaaatccttgcataaatctagaggaagacatggatgcacatgtttgctgcttactgtttgtgtcataaaggaaatagagttaacctgtggtccatttgaagggaaatcattcagtaaaatgtagtggatgggcgtcwtgaagtaaaacccagtagataatagcaacagggacagatctctcaagcataccactgaatttaaaaggggaaaaatgcgatccttagtccaatgccatttatataaatttaaaaatatatatacacacaaaacaattctacattatatagggatacctacaaatttgaggatacatattaaatgcattagaactgttgcc

SEQ ID NO: 193 - DNA, 401 bp, Homo sapiens
agaaatggaacgttactgtttaaagtcattaaaatccacatgaagggttataatatctggaagtagactagcttaagtttaagctgcaaattgacaattgtagaaaaaaacactgaacaagtaaagcaaaaagatctagctgaaaagccaataagaagacatatttgaatccaaaaaagataaatttataattctaaaagmaggaagaaaaaagaggaaaaagaacaaagaatgaataggacaaataagaaaacattgataattaaatttaaatgatgtaacactctaattaaatggcagagattgttgattgaaatacagaactaactctaagccgcctacaagaaacccgttttaaatataaatacacagataggttaaggccaagcatggtggctcat

SEQ ID NO: 194 - DNA, 401 bp, Homo sapiens
tgcctattcagggaggtcaaggaagacaaaggtctttaaaggaaaagcgagcaggatcacagtggttttgagataattctccttggctacaaacatcaataacaaaggtcattccagtctgaggttggacaggcaattgctgggcagttgtccttgcagaaatattttttgtgtaagggtgtgatggcctctgtgccgggytgtggtttttgtagtcctttctggtatcaggcatgcaagcataagtgtcctctcttcgtggccttccccaccaactctttgtcagggctttcttaacattagtgactccattttgattctgacaactttcacagcatgtatttagagcagccgctaggtgttcatccacagaggtggccgtgagcagttcctgggatcat

SEQ ID NO: 195 - DNA, 401 bp, Homo sapiens
gagacagagctggaacagtttcttgccaggacatatcatgggcaactggaacccaagtttaggcaagacaggaaaaaccaccacctgcaaattatcttttccctcaaatggataaacaggcgcagggtgcggtgaaagccgtcattccgttcagcagcagccacgccgctgagacggagcaacggccgagcatacgcagcygcactcaccaccgctggtacaggtagaccagaaacaccacgtcgtcccggaagcaggccagccggtgagacgtgggcatggtgatgatgaaggcaaagacgtcatcaatgaaggtgttgaaagcctgcagggccagacgggaggagggtgaaccccagttgctggggctggaatcctactgtttttggtaacctaaccaa

SEQ ID NO: 196 - DNA, 401 bp, Homo sapiens
accttaggtagtccacccgcctcagcctcccaaagtgctgggattacaagcatgagccaccgcgccgggccaacactgcttttacactacaaagcaaagttgaatatagcccacaataccacaaatatttttactatctggttctttgcagaaaatttgctaatccctgatttaaataatcccatgttcatagattaaaakactctattgttaagatgacagtcctcccccaatttattgataaatttctgccaatttaaaaaaaattaatgtcgttcctaagaaagtgagtccattccaaatactgcgaaatcaagggaaaaaattaaaaagtcagtcctggctgggtgcagtggctcacgcctgtaatcccagcacttgggaggccgaggcaggtggat

SEQ ID NO: 197 - DNA, 401 bp, Homo sapiens
actacattacttagtaggcagttttttccggtttctttccattatgtttattgatctgttgtgggttggttggttttgaccaaccagaattgatctattattgttaactagcgcctgtagttacacccgggctctggcgtgtgcggtgcctcctggggctgtggcgagtgtgcgatgccctgcctgtgcccctcacgccgycccctgcagagcagccctgccaccctgagcgctgtagctcgttctgtctgtccctgtcggggtgagctccatgcagtgtgtttacagaggcttggcgtttgggcctctaactggaagccatctttgttccctgcaggccatgaagtacttgtcatacctgctgtaccctctctgtgtcgggggtgctgtctattcactcc

SEQ ID NO: 198 - DNA, 401 bp, Homo sapiens
ccatccgtgggcactgagattcaactccagctctgccccagccctcatctgcctaggggccttatgggcgcccaggcactggggcagccaccagctcagggcaggggctcactcacttcaggactgccaggactgtacggaccccagtcagcaccagcctcgggagcgcctctggacaaggtgccaaaacgaagcacacgygctggccagccagcatccttcctcccagaggccggctcaggcccagaagtgctgccgtctgcactgaagacggtcagaactaggtatatgaccggggaatcctcaggcgaagagagtggctgcgatcagaaaccagtgctgcctccaccacggaggctcccctgacagaggcccgcagtgactcatctgcagatggccat

SEQ ID NO: 199 - DNA, 401 bp, Homo sapiens
gcagggcacaatgtcactggggagctcactcacctcagcattggccgcaccccttaaaccagccaccagggcctctgaagactgcattgcgtggacctctcagcttggccttcaggttgaaggctgacggctgaggaaaaggctttgtggaattttctaaaggcagaggttcaggccccaccccgggcctcggaattttcyaaatgcagaggctcaggccccaccctgggcctcccgcttccctccagggctgacatctgccctctcagtcagcaaaacctccctccagctctgctgtgccagggtaggagccagggatctggggctcccctcgggagggttgcatctggaccactgcaagcactgccctcacctccagtgccggccccagggccttgtcc

SEQ ID NO: 200 - DNA, 401 bp, Homo sapiens
atttgacccagccatcccattactgggtatatacccaaaggattataaatcatgctgctataaagacacaggcacacgtatgtttattgcggcactattcacgatagcaaagacatggaaacttttcttgaagttgcatctgcacagcaaacatactgttctttttaagttagagattttgttactagcaagtaaaaataragatggctttattcatgttttacataaaatagagaaaatatcagttttccatgttaaggaatactcttatttttacttggggatagctttgggaaggggtaatagagaatatggccatcaatcccccctgcaaagctaccccaagttaccaccttaagagcagaatagtttattttaaaataccatatttcatcaaacat

SEQ ID NO: 201 - DNA, 401 bp, Homo sapiens
gcttttcctgtgccctgccttagccagaaatagcttcttcatgagcagggccaccagctgaggtccccacactctaccagtgttggtaaatgtgggtaaacaccttaaattgcctcaggattcaatccctcccactatcctccaagaacagggtgaagttctgaatatatctctctatacaaatgcattctattgttgatrctttttccagagaggaggaaattaacagtaatagtaatcaatattattgactgcaacgaaatgtcttctttggaaattgactgatggtcacgtatcaaggggagatgctgggttttctctcagggaagacatttcactctgtcacctacttggtttcaaagagaaggaggtggaaggaggggccaaaaagtttaaggtat

SEQ ID NO: 202 - DNA, 401 bp, Homo sapiens
ccattgggtaaactttccacctgtgaggaacttatacattctagttttgttttctggtagttgctcctaacttgttaagatccgtagttacattctccccgtctatttcttcatcttaacaatacttacatctttcactgaacaaaaaaagtgcctgtccccacctgcctctatcctcaccttcttctcctagcccatcawcctaggttcattatgttgcaaattttattttgggttgttgtgcacatcaacaatggtagttctgaggacaaacatcaatcaatctttcttctggaggcagatgacttcaaagacatttctttggggtcttccttcttcacccaagtaaatttgaaagatgtatgctagagagtgggctgctttctcctggtagagtatag

SEQ ID NO: 203 - DNA, 401 bp, Homo sapiens
tggaaaggctctagagcacgatgggagaggaagtggatgagagataagtcatcagcttgaagactggtaaggggagaccccaccgatcctggtgttaaatcaagtttagcctaaagccttctccttacatattttaaattcaccctaatggtttctctgtacatagtgaactgtaagctaactgcacgtgtaaacaggctrtcacctactctcgtaccaagtagccgagtctcagttaatcacagcagccagacttcaaccactcacaggcggccagctgttgaaactggactcaaataagccaagcaccatgccatcaccaatccgtctgtttctgtacctcacttccagtttctgtacatcactttcctttttctgtccataaatctttgactatgagg

SEQ ID NO: 204 - DNA, 401 bp, Homo sapiens
tgacaaactcaaggacgtgtgttaagatgttatctttagttcctaaaactaaacatctccggactctaacttccttggctattgttttgggctactattaccttcctgcctaacaagttacttacttacttctcagggctagctggatgcctgaaatttcccttgaaggcactcaagattttcctttatttccatgcttgrtggtggcgttcggggtggtggcacaggtccctgcttcatctcaggcctgggtttaaattctcactctttctcaccatctgaaaaccctgaatagttgtttccatcctgaaacctcaatgccccactggtaaagtgggcgagagaaggcctgttttgttgtgttgctgggaaatccctgaggtaatgcatgtgtggacatg

SEQ ID NO: 205 - DNA, 401 bp, Homo sapiens
gaataaaggaagttctagattgcttgtgaaagtattagaaatatgtcacgtcattcccatgaccacagccaccatatgatcacatgctaaccacatcaatcctcagttcttaactaaatgaacacttttcaccacaagatttgcctttcaaaactattctacatatataccttcccttgtaaaacaattacctattgcctrcaactttttatacttctcttgcaacactccctaattttttaaaattactgactcctgttttaagctactcatcatactacccattacagtttttaataatgagagtaagatatggttcattagtgttgttaacacctgttatgttttagaagtgttaactaccgtgtctcttctaattatcttgaactagtaaattatta

SEQ ID NO: 206 - DNA, 401 bp, Homo sapiens
atgttgtcctggaacttaaccaaggaagaagtgtggaggtaaacagaaagagaaacaagaaaattgaacaaggacattggcttgggtccagcagtcacgagtgactgtcttaggctagtgtccgggcggtggtagagaccaaagaggttgagctgcgcattctcagccctatcctgttcaacattttgattagtgttacaratgaagacattgcttcatttattcaacaaatgttcattgagcacatattctgtgccagacacaacctggaagaaaagcttagcaaaccaatggatgagataaagctggaaggggtcgagattaatcctcaggcaggggagctaaacccatcagaattttttataaacagagctagggccaacttagcatattgtcaagcc

SEQ ID NO: 207 - DNA, 401 bp, Homo sapiens
agttaacgtatatactcatattttccttgaaaatgcactaaatggccaaccactgtccaatagaaacataatgagagccacatatgtaattttaagttttctgcttgatgcattagaaatataagaaacaggtgaaattaatttttataatatattttatgtaaacccagtatatccaaaatatttcaacatgcaatcaamataaaaaattactaatgagatatttcacctttctttgatgaagtcttcaaaacccaatgtgtattttacactgcacatcttaactagccacattttgagcacccagtagccgccggatgctgcggctccaaaccattcttgtgtctaggggagtggcaggccaggtctccatgaaggcaacctaactagatttcctccag

SEQ ID NO: 208 - DNA, 401 bp, Homo sapiens
tactaatgagatatttcacctttctttgatgaagtcttcaaaacccaatgtgtattttacactgcacatcttaactagccacattttgagcacccagtagccgccggatgctgcggctccaaaccattcttgtgtctaggggagtggcaggccaggtctccatgaaggcaacctaactagatttcctccagtgttgaagcmgttgtgcatttcttcagttgagcaccacagtgaagcagatggttcacctttagggcccaagaaggaaaattatctttaagtatcagagttgctcattgagcaaaacacggtcacttcctagaggcaccactgaacacgctggggaagggtagggcctcctcagccacttcacacatgacgtacatccctgaaacttagtt

SEQ ID NO: 209 - DNA, 401 bp, Homo sapiens
ggtggctgtgctgagggaactgtgtgggattaaccccggagaaacagaggctgcgtcttcacccaaggcatgtttatggcacaacaaatacaacaagcaggagctttttctggcagagaaagtttccttttttttttttttttgagtcggagtctcgctctatcacccaggctggagtgcagtggtacaatcttggctcartgcaacgtctgcctcctagtttcaagcgattctcttgcctcagcctcccaagtagctgggattactggtgcccaccaccacacctggctaattttttgtatttagtagagacggggtttcactatgttggtcaggctggtcttgaactcctgacctcaggtgatccacccacctcagcctcccaaagtgctgggattaca

SEQ ID NO: 210 - DNA, 401 bp, Homo sapiens
cattggcttgggtccagcagtcacgagtgactgtcttaggctagtgtccgggcggtggtagagaccaaagaggttgagctgcgcattctcagccctatcctgttcaacattttgattagtgttacaaatgaagacattgcttcatttattcaacaaatgttcattgagcacatattctgtgccagacacaacctggaagaraagcttagcaaaccaatggatgagataaagctggaaggggtcgagattaatcctcaggcaggggagctaaacccatcagaattttttataaacagagctagggccaacttagcatattgtcaagccaaagtgattgttttgtggcccaatgaattagcaaatgatagtaagtgttaactccaagtacttccaggaaagtg

SEQ ID NO: 211 - DNA, 401 bp, Homo sapiens
ggcgacagcgcgagactccatctcaaaaaaaaaaaaaaaaaaaaaaaaaagagagtactggcttttgggtcattaggactattatataactgaattgtgtcaggcctctgagctgaagctcagctattataacccctgtgacctgcacatatatgtccagatggcctgcaggaaccaagaagtctggagcagccaaaaaamccacaaagtaaaacagccagttcctgccttaactgagtaaccaaaattacaacattttaccattgtgacttgtccctgccctaccttagctgatcaaccaactttgtgacattcttcttctggacaatgagtcttatgatctccccactatgtaccttgtgaccccctcctctgctaacaatagataaccacgttttact

SEQ ID NO: 212 - DNA, 401 bp, Homo sapiens
ctcctggctgtgtcctttctctggccctcaaggtcccttttcccatctccctcccccgaccaggaggccacctcacacaccacggctgtgacacttccctgtgcccttccctcagggcctggggccatcctactagtgcaggagagggatcctcttcccccaggccgtcctggcgggtcctgcctaggtccggggtgccgkcccttggggagcgcagtgctcccgtccccgccctgtctccacactcaacctcgccaggtgttcagagcctctgtcccagccagcatgaggctggcatggttctgcctggtttaactctttgttcgggtgcagttggcacatccacacagtggctcatggccgcccttgcccagctctccaggcctggccgccggctgccc

SEQ ID NO: 213 - DNA, 401 bp, Homo sapiens
tgcctttcctggcgtcctgggttggagtggccagcagacagtggctgtggccttgaccactgtttgtcctgtggctccatggatctgcttcccctgcttgccctcagggcttgcaggaggaggaagacgtgttgaataagctggagtggttcttaaggtacagctggggaggaaacaaatccagacttgaaaagccacgcrcttatcacagaactggcataagacacgcccggaagcaaagctgtgctggccccgtcatccgacctctgcccacgttccatgctcatttgcaagtgtggctcagacacgtgtttgtggagctggtgtggggccagctgttcagttcagcagccttccaaacactttcctagctgctgaatgcttcattgttctttttaaac

SEQ ID NO: 214 - DNA, 401 bp, Homo sapiens
tggacgagcagacgagcctgctggtgctggtcccggcgggtgttggagccgccattgaggtgagttccgggcagtgacctgaactgtctgaggtccatgtgcctccacgcactcaggaaaggctttcagccccgggacctgagaccttctgtggaagcctgtgtgcttgttcccgatggcctcagtgttctggaagctgtrggatggcaggcagtgggtgtaaaggctttgaacaagtggagagcaaggaaatgcgtgttcgggtggtatcagctcatgaggctctgtccaccaagcagtggtgagtcctgaggccctgtccaccaagcagatagtcctgaggctctgtccaccaagcagtggtgagtcctgaggccctgtccaccaagcagagagtcctg

SEQ ID NO: 215 - DNA, 401 bp, Homo sapiens
gtgggaggatctcttgagtacaggagtttgagaccagcctgggcaaaatagtgagaccctgtctctacaaacttttttttttaattagccaggcatagtggtgtgtgcctgtagtcccagctacttaggaggctgaagtgggaggatcacttgagcccaagagttcaaggctacggtgagccatgattgcaacaccacacrccagccttggtgacagaatgagaccctgtctcaaaaaaaaaaaaaaaaattgaaataatataaagcatcttctctggccacagtggaacaaaaccagaaatcaacaacaagaggaattttgaaaactatacaaacacatgaaaattaaacaatatacttctgaataaccagtgagtcaatgaagaaattaaaaaggaaat

SEQ ID NO: 216 - DNA, 401 bp, Homo sapiens
aatgtacatctttcactacacttggggacttacctgctactatatcttcggattttttttctgctacttttttttctcatttcttcttctggagctcctattacagatatataagccttttgatattgtcccataggttcctgagattctgttcatttttttaaaaatttccctctttcttctttatattgagcaatttcraatgatctgtcttccattttaattcatttgtcattgccgttctgctgttaagccaacccggtacatttgtattttcagatactgtatttttcaatgttagtttctcactgggttctttttagtagtttctttttctctgctaatatgtcccatctttttatttattgtgagtgtgttttctttgctctaagcatagctgg

SEQ ID NO: 217 - DNA, 401 bp, Homo sapiens
tgtatttcttttagtagagatggggtttcaccgtgttagccaggatggtcttgatctcctgaccttgtgatccgcccacttcggccttccaaagtgctgggattacaggcctgagccaccatgtctgacctctctctctcttttttttttctttctaaggctgaagtgcagtggtgcaacctctgctcactgcagccttgmtatcctgggctcaagagatcctcccacctcagccccgcaagtagttgggactataggtatgcgccaccatacctggctaatttttgtattttttgtagagatggggttttgccctgttgcccaggctggtcttgaattcctgagcttaggcaatctgcctgcctggcctcccaaagtgctgaaattacaggcttgagcca

SEQ ID NO: 218 - DNA, 401 bp, Homo sapiens
cagaggtgaacgctcagctggggccctgcaggatcgctgccctgccctgcctccctgcaagccatcactgtcactccggatcacccactaggagccggccacttccacttaaggctctcggcacctctgacctctgaggcgggccctttccccttttttgttgaagaggaaacagtcctggggagagcagagggcttgctrgcgggggggtgcagctgggaaacggagaagcgagcacctggctgctttgatctctgcccaggccctgctgcctcctcctgactgcggctctcagaacagtccccctgtactgctgttccctcagaccccttgcagtccctaacatactgcctccttcacttctggggtcactgattgctcctatacaacctgcaggctgg

SEQ ID NO: 219 - DNA, 401 bp, Homo sapiens
gcaggcagctgggatgcagcaggtgcaggaagtcggcaccttctgtggctgggcccagcactgccagatggaggaggcagtgacacccttcggacacgcttggcagctcgaggggtgcctggaggccaccatccatgtgcactgatgccacttggctacgtggggcgccctgacagccgctctcagggcaacctggcaccwgctggttgtggctctgattccaccacaccctgacaccagcagtgcccgccatagcagagcaggtggattagagcacagccctcgctggatgttccagcacgtggagggtggcgggtcagggcttgaggcagggcaggtggattagagcacagccctcgctggatgttccagaacgtggagggtggtgctcaccgggtgtg

SEQ ID NO: 220 - DNA, 401 bp, Homo sapiens
ggacccaatccatttgagggcttcctggagtgggcagcacagcctagtggctaaaagaagactccaaagctgccctctgcctgcattcatgattcttgttttgatctttccctagctctatcgctttggcctgcatgcctcagtttccctatctgtcaaatgggaataatgatagtgactgtttcatggggctgatataaraattaaatgagttcatacacatgaggtacttagagcaatatctggcatgtattaagtgctgggtcaggcactgctattcttgttaccacaccatcagatgggagtcagttggctgaagctgtccatctggggctggggaggtcaggcaggactggagatacagatattttattttcactttttcaaatgatagttaataa

SEQ ID NO: 221 - DNA, 401 bp, Homo sapiens
ccttcttctctccatcaggccaaatcacggtgttgaccttggccacatcaatgtcttagaacttcttcacagcctgtttgatctggtgcttgttggctttaacatccacaatgaacacaagtgtgttgttgtcttctatcttcttcgtggtgactcagtggtcagcggaaacttgatgatagcgtagtggtcaagcttgtrtctcctgggagcgctcttccaaagatatttgggctgcctcgggagttgcagcgtcttgggccgccggaaggtgggtgacgtacggatcttctttttttgtgtggctgtggacacctttcaacactgtcttcttggcctttaaatccttcgctttggtttcggctttaggaggggcaggagcttccttcttcactttcggc

SEQ ID NO: 222 - DNA, 401 bp, Homo sapiens
cctatccctgagtagtttctctgtcggcccctgcaattccaacctctttctctgtttgctggagtttgctggtgtgctgctgacaaacctcaggacagcaagttctgctaaaatgccccatacagagctgtgtgagctgtggcaccagctcgtggtcacctcttgtgaagcctcatgccgctgactccttgcaggcattcwggagatggtggacaggggcaggcagctgcagaaggctgccctgtgtgtctgtctgcagatctgtcttgttagaggcccggggcaggctttgcggcgcagctgtgccagtggtggcctgcgttccagtggttatggagacacgtgtgccgacagcagcttttcccccaaatcacagtctctctcatgtgtcgctgttgctg

SEQ ID NO: 223 - DNA, 401 bp, Homo sapiens
tcagcggaaacttgatgatagcgtagtggtcaagcttgtatctcctgggagcgctcttccaaagatatttgggctgcctcgggagttgcagcgtcttgggccgccggaaggtgggtgacgtacggatcttctttttttgtgtggctgtggacacctttcaacactgtcttcttggcctttaaatccttcgctttggtttcrgctttaggaggggcaggagcttccttcttcactttcggcgccatcttgtgaaaagggaaagtttcctttctaataccattttcacttctcccgaattttgtggatcgtttcttggtatctaccccagatttcaggagtgttggctggatcttagggattgtgaagtcttcatttccctgtggtgagatctgaggcatgat

SEQ ID NO: 224 - DNA, 401 bp, Homo sapiens
ccttcttcactttcggcgccatcttgtgaaaagggaaagtttcctttctaataccattttcacttctcccgaattttgtggatcgtttcttggtatctaccccagatttcaggagtgttggctggatcttagggattgtgaagtcttcatttccctgtggtgagatctgaggcatgattttaaacagtgtgagggaaggaratctccaggcactttaatagaatggagaagcaggatgggatttgagaggaaatctgattttgaaaaaaggagaactagagttgagttcgtaattaactagcaccttaaaggtcattcagcatgcccatctgcacagtgggtgtaatcaccctacagaacaaaaacaaaaaggcaatggagaggaagctgtaaagcactgt

SEQ ID NO: 225 - DNA, 401 bp, Homo sapiens
ttgcagtgagctgagatagcgccactgcactccagcccgggcgacagcgcgagactccatctcaaaaaaaaaaaaaaaaaaaaaaaaaagagagtactggcttttgggtcattaggactattatataactgaattgtgtcaggcctctgagctgaagctcagctattataacccctgtgacctgcacatatatgtccagayggcctgcaggaaccaagaagtctggagcagccaaaaaacccacaaagtaaaacagccagttcctgccttaactgagtaaccaaaattacaacattttaccattgtgacttgtccctgccctaccttagctgatcaaccaactttgtgacattcttcttctggacaatgagtcttatgatctccccactatgtaccttgtg

SEQ ID NO: 226 - DNA, 401 bp, Homo sapiens
aatccattgaaagcgtgagaggcaggtagttcctgggttctgtgcatagttgctttaagctgtaaacacacaatccttgaccaaataccctttaatccacttgacaaacaagaagcttccgggcctccaaggggatgcccaagtctcccttttctctgggacaaaacctggttttgtttcttaggtttctcagcggctaayctggaacatgtttgcttttgccccgtttctttgtcccagtgttttttctgctgttttcacacctctctggataagctttccaagttccttacagaaaaccagaaagattattcaccagcagtcttcctggtccctggcttgtcagcctcaatgcattccagtgcactcttgtgacactgtatccagagtagctgtggctc

SEQ ID NO: 227 - DNA, 401 bp, Homo sapiens
tgtctgggtcgttcattcgtggctcctggccttcgaatattaaaggaactatttcctgatttctcccctcagctgtggaaagtgaagaaggcattgaagatgactattttttggagaggcctgatgcccgaatttcaggtaggatttagttgtaatggctgaaccccaagcctctctgaagagtgtgattttgccccctgygcaaagagtaagatggccatctgcagatgagtcactgcgggcctctgtcaggggagcctccgtggtggaggcagcactggtttctgatcgcagccactctcttcgcctgaggattccccggtcatatacctagttctgaccgtcttcagtgcagacggcagcacttctgggcctgagccggcctctgggaggaaggatgc

SEQ ID NO: 228 - DNA, 401 bp, Homo sapiens
atattaaaggaactatttcctgatttctcccctcagctgtggaaagtgaagaaggcattgaagatgactattttttggagaggcctgatgcccgaatttcaggtaggatttagttgtaatggctgaaccccaagcctctctgaagagtgtgattttgccccctgtgcaaagagtaagatggccatctgcagatgagtcacygcgggcctctgtcaggggagcctccgtggtggaggcagcactggtttctgatcgcagccactctcttcgcctgaggattccccggtcatatacctagttctgaccgtcttcagtgcagacggcagcacttctgggcctgagccggcctctgggaggaaggatgctggctggccagcacgtgtgcttcgttttggcacctt

SEQ ID NO: 229 - DNA, 401 bp, Homo sapiens
tcacttggggccagattcgagaggtgctgtcttgagagaggaagcaggcagctgggatgcagcaggtgcaggaagtcggcaccttctgtggctgggcccagcactgccagatggaggaggcagtgacacccttcggacacgcttggcagctcgaggggtgcctggaggccaccatccatgtgcactgatgccacttggctrcgtggggcgccctgacagccgctctcagggcaacctggcacctgctggttgtggctctgattccaccacaccctgacaccagcagtgcccgccatagcagagcaggtggattagagcacagccctcgctggatgttccagcacgtggagggtggcgggtcagggcttgaggcagggcaggtggattagagcacagccctc

SEQ ID NO: 230 - DNA, 401 bp, Homo sapiens
gaggagaaactccaatgaagaatcacttttcatgatcagtaaaaatgagatgatagacaaattttagttttcaaaaataaggttgtattggaatacaaccacacttgtttatgtataaccattggctcttccaaactatagtagcagaataaagcagttgtaacagagatcatacagcttgcaaaacctaaaatatttacygtgtggcccttcagagaataagcctgcacacaaaatgagtagataatggcctcctctaaacaatcagttaaaatgttgaatagagtgagggcaagggctgaagtctgagctgtgtgcacactctggagacctcttccagttggattcaactcactaagaccaactcttggggtagtccccaactcttagctcccatttct

SEQ ID NO: 231 - DNA, 401 bp, Homo sapiens
tgtttgtttgtttttagtaaatatggggtttcactatgttggctaggttggtttcgaactcctgacctcggcctcccaaagtgctgggattacaggtgtgagccaccgtgcccggcctcactgtgattttgatttactttttgtcagtggctaaagatgttgagcatcttttcatgggcttgttggccatttgtataacaytggaacatgttggttattgagctttataccctctgtgccctcgaggctgtgagctcaatgacagtggaggacgccgctccttctcctttgtgtcttcagtgcaaagggcaactgacatcgtaggggaaaaaatacccaaaacctcttctcttaaaaatgagccgaatgaagtaaagaatagcccaagagattctgccaat

SEQ ID NO: 232 - DNA, 401 bp, Homo sapiens
tgagcacaaaatgaaggtattgtcagatgtgcaaagtgtgagtttacaacctgtacaaccttgatgaaaggataagaatgtatttcagaaagaaggaaacaactcataagagtggagttcaacaaacaatggtgagcaaagaagctgggaacacgctgattaaggtgagtggctagatacagaaaataatgaccatcaccwatatggggagttttctgaaaatgaaagaataatttcagaggggagaccagttccaggaaagtttatccaagcagatggggagaccttgagccatctgaggagtctgagatttcctaggcatgggcctgtcttcatatccctgctggcctctggctagaagaagcctgtgaggggtgtggtctttccgtgaatgtggtggc

SEQ ID NO: 233 - DNA, 401 bp, Homo sapiens
ccctccgggtccacatgccgacttccctcaccattcttcataaagcatggtttctgtcatgtggtggcatctggaagatcagtgtttaaattccactacagagtagctgtgcgactcatttctctgaacttcagtttcctcgtttgtgaaatggggcagttatttcttctttcctggattgttgtgatgattagaatgagsctgtacgtgcaaagcaactagcacaatgtctgggtgcaagagttttaaaaagtgaagctattgttattctaacctgggcctctagctcacttatattggaaagcccaaacagaaaacactcattttgaccagtaggagaaaccctcccctgcttttggcctggctgcactcacagaagtctacgaagactgaagactgtg

SEQ ID NO: 234 - DNA, 401 bp, Homo sapiens
atgtgtggcaggtacttcacagggggacgtgttctcattttccttggctttctaataaactctggtctaaagaaaatctcttctataaaatgagtggccagtgccaaggggtagagacagaagctccttctggacactggctgctccagtagagcccatcctgcccaggtacggagcccaccagctgcgatcaggacactrcagttccaccgtgggggaccccgggccctcaccttgtcagtcctctgtttcagcttcgagggctttgacacagtctaaaatccacaggtacttagcgcatttctaagtcccacctcatgatcaatcataagacagggctttgatggtatgcagaaatatcaatgaaatccaagaggaaaacagaactaaactcatcggga

SEQ ID NO: 235 - DNA, 401 bp, Homo sapiens
atgatcaatcataagacagggctttgatggtatgcagaaatatcaatgaaatccaagaggaaaacagaactaaactcatcgggatgaatgacagataaaccctttaagcatgtttcaagtcgcccaggtggagcctctgttggtcagtgaagcttcagggttcccatgagatggatctgcatgaggctcagtcctctcttraaacactgcccatttgcaggagcgccggcccacagggcagaagtcagactgtcagagcaagtgccggtcctgaagctggaaggactttcaaggctcattccccaaactcatttttttttaaacctctgctcatcttaaagaaaggagaggcaggcgccccagaaacctctcaggagtaactgacacacgtgacttgcggt

SEQ ID NO: 236 - DNA, 401 bp, Homo sapiens
gtgaaggctaacgggggtcacttttatggggtgcttcaccgagtgcctggcttgtgcacgtgtcccttgtatgccaagtatttatattattcttgtttcgtgtactttgaactccgacatctgcaacatatacttcttgattatacgcatcttgattataaaacaaccaaatgcatctacatccggaactgtcttgcgcaygaacttctaaacccaaattggaagagacagctacagatggagcacgcacagctacacgcaaacatacacatatccttctaacacctgcagctcctggccccagaatgcagacacccaagagctacccttagataggagtctatactgggctacctacagaagaacccaggtcgccgctggcggcctcgcagctgcacccc

SEQ ID NO: 237 - DNA, 401 bp, Homo sapiens
caggtacgtacaccttcagctgaattaggaaagggaattacagtggatcctggagcccacttcggtttgaatcctggctccataatttcccccagaggatcttgggtaagtgatttaatggtctatgcctcagtttcctctcttgtaaaacagagataaagatcatatatcgtttatggggttaactgtgaaggctaacgrgggtcacttttatggggtgcttcaccgagtgcctggcttgtgcacgtgtcccttgtatgccaagtatttatattattcttgtttcgtgtactttgaactccgacatctgcaacatatacttcttgattatacgcatcttgattataaaacaaccaaatgcatctacatccggaactgtcttgcgcatgaacttctaaacc

SEQ ID NO: 238 - DNA, 401 bp, Homo sapiens
ttggcatttcggggtcggccttaccagccaccatccaccgagaattgtgaaatttataacgacagtcatcagcagctataatgtccatcaataaaatgtatttggcttttttatccagcccagaacatctcactttaaatggaggaaacattcgcctataaaacgaaagaatagaaaagaaaaagaaagataaaccacccrtaatcttgggtttgatttcctatgtatcacataatattgaaaccaagtctaagactttatattgtgactgataagcttacatgtttcagagtgaggcaactggttgcaaaatcgttttcttccacaactaacgtattaaatattctcagtttcgccatatttagacgtcccaaggttcaaaacaaatgcattgtggcact

SEQ ID NO: 239 - DNA, 401 bp, Homo sapiens
tgtcttgtgagtttgaggaggattttatgccaaactttggcctcatttagcttggaaggagacagaacgcttaagatatcatgaaggggattttatttttgtatcaactttatgcctttacaggcttcaatgctattacaaaaaaaaaatcagtgattttaaatgactattacaggatcttctcttccaggcaggttttgrtcaggcaaggacctttaaacattatgtaaaatatggttgataatattcacacattaaaaaaaaggtgtgtgtctcctcactggctccttaagtctgcaaacataatcttaaattatgcaaaattgcctgctgaactcgcccttgggttacagacacgtgaagtagtgtgggcaaacaaaccaaaatgcccttcgatttct

SEQ ID NO: 240 - DNA, 401 bp, Homo sapiens
gctgcttgccaagaggtagtatgcaaacagacacccacctcccctccccaccagccaaataaggatctgggagaaaggcctctgtgctctccttctactgccttagcggggaaggggagacacacctgatagccctttttgaactctggcttctgtttaaggatgtttagaaggcttctagcttttaaagggcaagtgccytggttggtgccccatcccccttcaactcttccaggccacgagggactggagtccagtgatcacaaaggtgacatggtttacctttgaggttcgatgtccctacagtggaggcggctggagaagaagcctgggcgaagcagttgaaagctgcttgttcactggaggactcatcagaggtcccattctcctttttgtgtctt

SEQ ID NO: 241 - DNA, 401 bp, Homo sapiens
agacttatcccatccactgttaataaatttcttctgaccaccggtatccccttcccccaaccccactctgcgcttccttctgggtcccccatctctggaaggtttactcttcctctaggcactcggacagaccctgccccacctcctccacctgtcagcctccagcaccatgctaccagcccctggatctctctccatcawcaccaaggacccctgtcccaacttttttttttttttgagacggagtctcactctgtcacccaggctggagtgcagtggcacaatgtcggctcactgcaagctccacctcccaggttcacaccattctcccgcctcagcctcccgagtagctgggactacaggcgcccaccaccgtgcccggctaattttttggaaatttt

SEQ ID NO: 242 - DNA, 401 bp, Homo sapiens
ataggaagaaaaaacagggaaatcataagtaattcatatacttattgaggactttgtagatattaaatgagtgaaagcctgcaatatgctgcacacagtgtgtgcagcccccaggaaatctccttgcacctgttaaatggggatacagattcagtttaccccacagaggtaaccaaggggaagccggaggacataaattaktaggaataattttttaaatctttcttcttccccaacccccatcctcccaccccaatcctagaagtttctccaaacaaaacaaaacaaaacaaaaaaaacagacagtgaagatcttaagcaaaaggaaactggggctgggggagggaaaaagggcatttgttataaacagagacaaagaaatcgtccagcatgattcactg

SEQ ID NO: 243 - DNA, 401 bp, Homo sapiens
cgcccagcaggtttgttttttgagacaaggtctcactccatcacccaggcaggagtgcagtgataccatcatggctcactgcagcctcaacctcctgggctcaagagatcctcctgcctcagcctcccaagtagctgggactacaggcatgcaccaccacacccggttaattttgacattttctgtagagatagggtctcrctatgttgcctaggcaggtcttgaactcctgggctcaagtgatcctcccacctcagcctcccaaagtgctgggattacaggtgtgagccaccccttcccccaccttgctgtaattatttacttagactctaaaccaaagaacaggcttcaggccaggtgcagtggctcgtgtctgtaatcccatcactttcggaggccga

SEQ ID NO: 244 - DNA, 401 bp, Homo sapiens
gtgtttgctgtttgtggtgtatatgtgtttactatttatgtgtgtctgtggtgtgtgtgtgtacagtgggactagagtgtttgggcgtgttgtgtgtgtcatttgtgggtgtctacatggggatatcttgtgcgtgtggcatgtagggaaggaaatggtgtaatggcatgtatgtatagtttgtgtgtgcattcagtgggwggtgggtgtgtgtgccatggctgtgtgtgtatggtgtctgtatatgtgtttgctgtttatgttgtgtgtgtggggtgtgctgggtgtgtgttgggggagtggggagtgtgtggtgcatgtgtgatgtgtgtggtgtgtgtatgggatgtttggtgtgtctttggtatttttggtgtatgtgtgtttgctatttatgtatg

SEQ ID NO: 245 - DNA, 401 bp, Homo sapiens
tcaccagttcccagattggctgttagcagttatggggtgggaggagggactgaagacccctgctctgcaatcctggacttcaaagagagtccattttacctgacaacacacttcattttgaactcactgtcattgtcactgtccttgggtcctctgtggacttcatgatggggatgttccagctaaatttctttagtgtgwataccaaaacatgatcttctctccctgtgaaacctgaagtcttcaatagagcaatttattccaagaacatgaatccaaccaagggtccccctttccacctctgagtaactctgtgtatataacttcttcttcccaccaaggggaagggatttgaaagattacacactatagcatttttctcaaagtgcaaaatgcatgtg

SEQ ID NO: 246 - DNA, 401 bp, Homo sapiens
ttttcttctcagacattacactttttgtctgtagatgtttgacaaggatcttttgctatcttccatgtctctgccttaacatcttaatctttcctctagcgacttgaacacgcggaataaaggtgatcaacgggtttaatgtccttttctactaattctgtcatgtctgtcgctccccggtaagctctgggtggttttctycccattgcaggaattgtttccttgcttcctcgcatgcctggtaatatggatgcacgcagtgtgcgtttcatcttgtttggtgatgtatatttttgtgttcctaaaaatattgtttagttttttcctgggaaacagttacattccttggaaaaactcgatccttctgaatcctgtgcttgggctccagtaagtgggaccag

SEQ ID NO: 247 - DNA, 401 bp, Homo sapiens
tctggttctgtcttgtggccaagtggaccatggggctatcccaagaaggaggaattcagaaataggggaaggttgaggaaggacactgaactcaaaggggatacagtgattggtttatttgtcttctcttcacaacattggtgctggaggaattcccaccctgaggttatgaagatgtctgaacacccaacacatagcacwggagatatgagctcgacaagagtttctcagccacagagattcacagcctagggcaggaggacactgtacaccaggcagaatgacatgggaattgcgctcacgattggcttgaagaagcaaggactgtgggaggtgggctttgtagtaacaagagggcagggtgaactctgattcccatgggggaatgtgatggtcctgtt

SEQ ID NO: 248 - DNA, 401 bp, Homo sapiens
tccgcccgcctcagcctcccaaagtcctgggattacaggtgtgagccactgcatctggccattgaacatctttttatgtgcttcttggccagtagaggacccttaaaaaacttttaattttgacctagtttcacatgtacagaaaagttacagaatagttaaaacaaaaaactatatacccttcacccagattttccagcrttaacactttgctccatttcagctattatttcctctctctctcactctctttctctctcttttttttttttttttgagacggagtcttgctctgtcgcccaggctggagtgcagtggcgtgatcttggctcccgagttcacaccattctcctgagtagctgggactacaggagcccgccaccacgcccggcaaatttttt

SEQ ID NO: 249 - DNA, 401 bp, Homo sapiens
acccaagcccacggggaggccggccccagagaagctgctgggagggagggagggtgcaggctaggggcaacgacagtggcagcgagtcaaagacacacccaggccacacccagaaggccactctaccgaagcagctgtgtcgctgtggccgcctctaggccttggcagcggccccaagagctagacgtggtgaaccgaagrgggaggcagccgtactggacaaactcctgccaggcagctgcgcggacttggtgacgaaggcgaaaccgctcgtggcctgcttgttggttcttaatggactgacctttattaactttaatatctcatctgcccggccctgaggcaaggtactttgatctctggtcattcggtgaccttgaaggatgctggaagactcagag

SEQ ID NO: 250 - DNA, 401 bp, Homo sapiens
ttccagaccccacttacaacacacgactaagtaacacagtacaggccgtgtattcgtatttgttttcctgtgccaactcatctggtatcttcgtattttaatgaataaaactgaaagatgacaacttgaagccccagtgcattcctctcaaatcgagtgaagtttacaacaccttaattataaaagatttatctgatccaraagaaagcagagtgtacagaaacttcactgtcatctgcgaatggactgagagcatctggatttggttctcagcctgaatgagcatcctggaatggtgtgtctcgtccatttacgtggagacacatggaacaggcctgcaggcttggggctctgagcagggctcccaaagcccctcagctgcaaggaagccaggcccatga

SEQ ID NO: 251 - DNA, 401 bp, Homo sapiens
ggcttagagcctgggggccaccatttacaccctgggtgtggctgattataggaaagacccagtaattccaaacaggttaaccaggcaccaatatggagccttgtgatcaggttagggttagagtagggaaaggcatcccaggtggcttttccagggccaccctgggaagggtcatctaatctgccccaagtgagttgggcyaccccatgactattgcagaaatgacacgtgtcgtataaatcccaccacaccagaggggagggcggaagacactccctgagagtgggctgctggctggagaaagctgtggccctcagagcctggcattctgctgcccccaaagcatgggagcaggacgctgtcctctgggctgggggtctgctgcatcctcacccctgggc

SEQ ID NO: 252 - DNA, 401 bp, Homo sapiens
gtgagggccttgccagggtttgaggctacacatttggcaactcctccccacagtcctataatcagaaaacccaaaacctgggcagcgggagctgacctgaaatgatgcaatgcttttttttttttttttttttttggagacaggatctcactctgtcacccaggctggagtgcaatggcattatcatagctcactgcagcytccatctccctggctataggaattctcctgcctcagcctcccaagtagctgaggcaggcaggggcacaccatcatgcctggctaattaaaaaaaaattaattttttgcagagatggggtctatgttgcccaggaactcctgggctcaaacaatcctttcgcctccccaagagctgggattacaggcgtgagccaccatac

SEQ ID NO: 253 - DNA, 401 bp, Homo sapiens
caggtctgcagtcaggtttgtacccacttttaattacatgtagactaaggggcggtttatgcagaaatttctagggaaggggtagtaaccgttgtgtagtggggtcattgccatggaagtgggtggtaacgcctgggggttgccacggcaatggtaaacactggtgggtgtgtctcatggaaagccgcttccgccctggcygtgtttcagctagtcctcaattgggtctggggtccaagtcccacctctgatctcataagaagttgttacggattgtactgtgtcctccccaaaatctacgtgttaaagtcctaacccccagaacttcagaatgtgcccttatttggaaatagaatatttgcacgtgtgattagttaagatggggtcattagggtggccct

SEQ ID NO: 254 - DNA, 401 bp, Homo sapiens
agccctggtgatggtggtgttggagccacaggtcttcaaggagagggaggggcgtgggtggctggggccctaggcccatctggctactgtcttctgcctatcttgtagaagctccttgtacactgagttccttcatagttttactgtgatgagacatgtgctatgcagtgggcctgaagtttacttaggtatgctgggaastaggcgtcagcttttgcttcctgtgggacagacaggcaccacctcagtgatccctcctcctccttgcttctccctctaactgtgctttgctccactgaccctaattgtctcttcctctaccaatgcaccttcatggtttaatttggaaatttcatctgattttccttagactcatacataatcgtaacactgtaatgcac

SEQ ID NO: 255 - DNA, 401 bp, Homo sapiens
atcgacactcatacatacaaattcataacacagacacacaccaatgcaataccatacatctacaaacttataaacacacacacacacacatcgacactcatacatacaaattcataacacacacaccaacgcaataccatacatctacaaacttataaacgcacacacacacacatacatacaaattcataacacagacayacaccaacacaataccatacatctacaaacttataaatgcgcacacacacacacacactgacatacatacaaattcataacacagacacacaccaacgcaataccatacatctacaaacttatagacacacacacacacacacatcgacactcatacatacaaattcataacacagacacacaccaacgcaataccatac

SEQ ID NO: 256 - DNA, 401 bp, Homo sapiens
tcgctccggtgacccttcgacctggattcgagcccccatgtgtgggcgccacttgctgagaccagctcggttgtggagaccctaacccagcggcactagaggaattaaagacacacgcacagaaatataggatgtagagtgggaaatcaggggtctcacagccttcagagttgagagccttgaacagagatttacccacayatttattgagagcaagccagtcataagatttactaaaagtattccttacgggaaacaaagggatgggctctggctagttatctgcagcaagagcatgtccttaaggcacagatcgctcatgctattgtttgtggtttaagaatgccttaagagaccctctctaaaacaaacaaacagctctcccctctcccctctttcca

SEQ ID NO: 257 - DNA, 401 bp, Homo sapiens
atacagccttcccactaaagttttagacaacttcctgtttgtgagaagccatctcatatatgacaaacccagatgaagaagtagattagttaagatctgggtaaggacacccaagatttttcccaactgaagccacgtgaagctctaaactgcactctaaaacaagttccacttctggagaagggccgaatacatggacartggtgggtaactttgcagattatcaatttcctacctccaaactagttttgtaagcagttgagatcaagatatcaatcatagaaacagctttagagctagaaatgcaccttagagtcacaagaagcatttgattgttcagagttagaggaggttggcagttttagaacaatgcgagagggggacgctgaagcccagagcag

SEQ ID NO: 258 - DNA, 401 bp, Homo sapiens
gcagaaggtgaaatagctgtgaaatttaccacatcaacaaaacgggaaacaggaaccaagcggttatctcagaggatgaagaacaagaagtgggggcgtcaacatgccacagcaccctgggagcagaaggaaacctccatagcccgacaaaggtgtccacacaaacaccctcggctcacctcagtcccacaagatcaatcycagcatctgtccctactgtctggaattaagggtgacctcatgccatctgggtacccagcatgggtggtgacagccttgtgtggtcctctgtgatgacccctgtctgtcctgatctccacctgtgcgcactgggactccgcatggccggcatttatgcaaatgcggctgctggagaggaagagagccctgggtgtcctccc

SEQ ID NO: 259 - DNA, 401 bp, Homo sapiens
cccagacgggagaaacaagtgcaaactggctgagttcattgctgtccatataaggtgtattccaaacacgtctttctatgccagcacattggcagaacattaaatctttgtatgcaaaacaaagaaggccattaaagtaattaattatatatagattatgtaattttttaaaatcccatttgccccaattattggctcaawtgagaaaatttaggtgggattgttgggaaaattatttctatattttatcccctttgtaccctgaagactgtttgcaaggacttgaagagctgagaacacacaggccccaggaatgcccatgcctctcttatcttgaatttattttcttaagtgctctaacaagagtagcaagtgtgaacatctgtctcaacatgtagtcg

SEQ ID NO: 260 - DNA, 401 bp, Homo sapiens
cagaatcctgacgaataccaaaacgggtcctgccacagggtatcacaaagattgggctctacaaagtaaaattgccagggtataagtggaccctcagggaggctggcttatgccaggctgggaattgggcagtcctcgaacactgaacacttttccatctgtcccttcatgcctgcttgtcttggggaggctggcttttakggaggggtccctgggaaggaatgggggccgatggggaggggaatgggatccgtattgcgtatttggggtgaggaagggacccagagggaaagtggaaagaacaggagccggtggagggcaatgtgcagggtaggggtgggggtgcagttttcacctctggtctctgtgagatacgaaataagattgtgttctgcgtatga

SEQ ID NO: 261 - DNA, 401 bp, Homo sapiens
aaaaaattaaaataaataaaaagaaaaataaataattaaaagaaattaagttggttccatctataaggatgacagttgaggtaggatcaacactgaaaactgatcaggcaggtttggacagagctggctgtcacacacccggagagcactgctcatttcatttgccttcctccctgtgggcagctggccacacctgctctsagcatccctttctcaacccctggggagcagtagatgagctcatttcagcagtgatatgggttggtcttgccaggatactttctacttgggaatacctgagataacccaggtgaaatcctgttctgggcagctgctttgcaccaggcaccctgctggtcactggaaggcacacagacaggaataggtcacagtcactgtct

SEQ ID NO: 262 - DNA, 401 bp, Homo sapiens
ccagacagccagttggctttgattagcccatgcatctgcaaggagccatctcatgtgaaccaggatcaacactgacagccagtcgcattctcaacatctggtttacttccagctcaaaatgggaactttccattgcctctggtcacttgaatcctctagtccccaaaggaactcaactaaccaggcatgctaattggatcragtcaggaaccaaggtttcaattagatcagggcgctcctcttcatgacagcccgaaacaggcattcccatggggtgaatttagtcttaggccaggcatccaggggaaaacaagtccttaatttgccaaagccagtccaggatccagttggattttcattcagacacatgcctggattgccatactgaacactccctggcc

SEQ ID NO: 263 - DNA, 401 bp, Homo sapiens
tatattgtcctggctggtcttgaattcctggtttcaagtgatcctcctgccttggcctcccaaaatgctgggattacaagtatgagccaccatgcctggccaaaaaataccattttaaactgtatatatcactgctttatagcatattcacagagttgtctaaccatcatcacaaccaatgtttgaactgttcatcatttsaaaaagtcactccataacctttagctatcaccccagtcccctattcccccatctccaagcaaccactaatctactttgtgtctctatgaatttatctattctggatagcttatctaaagggaattatataatacatggccttttatgactggcttctttcacttagcacagtgttttcaaggttcagccatagtgtaaca

SEQ ID NO: 264 - DNA, 401 bp, Homo sapiens
gactgtgcagcagagcccaatattacccagaccatagctcacacatgctcttctgcacccagcagaacacttctttattcctctatggtggatttaatcttatgagcctccatccagagggggagcagggcaagtataaggtgaaagtggacttcactctttttttttttttttgagatggagtctcgctctgtcgctcaractggactacagtggcccaatctcagctcactgcaagctctgcctcccgggttcacgccattctcctgcctcagcctcccaagtagctgggactacaggtgcccgccaccacgcccagctaattttttgtatttttagcagagatggggtttcattgtgttagccaggatggtctcgatctcctgacctcgtgatccgcc

SEQ ID NO: 265 - DNA, 401 bp, Homo sapiens
taggataagtagggttaggatgtaatagttactattactattgttgtctcttggacgcttgggtggggaatctactctagaaggggtatttcagggaaggcacttgtgtcctgaaagatgggatgcatgcagctgtgggaagagcaagtgcattccaagtagagaggcagtacatgcaaaggccctgagaccagtgtgatygcagcaccaaaggcatcaggtggcacaagatgtggctgaagagctaggtcaaggccaggtcatgtgtgtggccttaaagatcttgctatggagagcatctcatgcataccccaaatgtctcatgcgtggtgcctgacacatagtaggtgctcagtaaaagattagtgaatcctcaagctgaattacaattaagcttgttt

SEQ ID NO: 266 - DNA, 401 bp, Homo sapiens
tatagaatgatgacagggcaagcttggatcaatgtcttcaggagcactatggaaattacaaattttattctgaaatctatccagctataccacgagggctaaagataatcatttggcctctacctcttcctgccttcaaagtaggaggcagagtttctgtggcagagataatacggcatgctcaccaaacctgcgtcttcytcctggccacccaggaagcctgcattcccaacatcctttgcagtttggttgagaccgcatgaattttagtgccattttattcactccccaccccacttccctctttcaggccataggcatgggctgtgaggtgaaatcaaaggatggaggaagcctgggcacagctcagaaaagaggcaatctccccagtagagtcaacc

SEQ ID NO: 267 - DNA, 401 bp, Homo sapiens
ccccttgatgagatggcaagaagataaagagagctttgatctctgacttagattctgatcgtcctcaccagatggaagagggatcttgcagaacacaagcatgagtggctcaagaattaaataaataaatttttctcaaggaaaaaagatgacgctttgtgactaagtcacaaggatctggaattgtatcggttactgcgstatagcttagcctgctctatcacactactatgattgtttccatctcacagataagaaaactgagactcaaagaagacatatgacgtgccaaaaatcatcgtgctattaagaggcaggactgagaatagaactcaaacctcttgattcaagtgatctttcccttacgacccaaatgttaagctgttgcctgcatgcataaa

SEQ ID NO: 268 - DNA, 401 bp, Homo sapiens
aacgggaaacaggaaccaagcggttatctcagaggatgaagaacaagaagtgggggcgtcaacatgccacagcaccctgggagcagaaggaaacctccatagcccgacaaaggtgtccacacaaacaccctcggctcacctcagtcccacaagatcaatcccagcatctgtccctactgtctggaattaagggtgacctcwtgccatctgggtacccagcatgggtggtgacagccttgtgtggtcctctgtgatgacccctgtctgtcctgatctccacctgtgcgcactgggactccgcatggccggcatttatgcaaatgcggctgctggagaggaagagagccctgggtgtcctccctccatggggacagcacgagactgggtgctgctggcccaga

SEQ ID NO: 269 - DNA, 401 bp, Homo sapiens
catcaaatgaaaacaatggactgggaaagttcatttctatcacaggaaaatttcaagcagttatgaagcccagatatttcaatgatcttgggatgattgtagttaaatttctttgtgtggctgcgcctaaacccagacgggagaaacaagtgcaaactggctgagttcattgctgtccatataaggtgtattccaaacacrtctttctatgccagcacattggcagaacattaaatctttgtatgcaaaacaaagaaggccattaaagtaattaattatatatagattatgtaattttttaaaatcccatttgccccaattattggctcaattgagaaaatttaggtgggattgttgggaaaattatttctatattttatcccctttgtaccctgaagact

SEQ ID NO: 270 - DNA, 401 bp, Homo sapiens
aggggtgggggtgcagttttcacctctggtctctgtgagatacgaaataagattgtgttctgcgtatgaagtaggcagttgggtgagaggtttaagagaagagatcatggtttggaaaggctctagagcacgatgggagaggaagtggatgagagataagtcatcagcttgaagactggtaaggggagaccccaccgatcytggtgttaaatcaagtttagcctaaagccttctccttacatattttaaattcaccctaatggtttctctgtacatagtgaactgtaagctaactgcacgtgtaaacaggctgtcacctactctcgtaccaagtagccgagtctcagttaatcacagcagccagacttcaaccactcacaggcggccagctgttgaaactg

SEQ ID NO: 271 - DNA, 401 bp, Homo sapiens
agacattcaaggtcacaggaccaggacccctctccactgggcaatgtggatgcatcccctgaaggttgaagccatgcacactcccacccacacaggggcaggatttgcacagccagaccaaatgcccacactctccccatcacagtcaggaactgggccaaaggcccccgtgagaccaggggaatgcggatgtggtggacraggggcttaacaaaggaagggtgaggtcgtcctccgaagggcggagggtacgggctgaacagctgggcaccgaggctgcacctggccagtccaagccagaggaaggtggaggccccagctgggcgcagcaggcaggcgcgaggcaggtaggtgagtgcacagagagagccggaggggaggctggaggcgtgggggtctca

SEQ ID NO: 272 - DNA, 401 bp, Homo sapiens
caaaaacagagcatcacttacgacgttttcttgccaagagcatttaaagtaaatctaattaagccccaagacctatggttcccaaactagatgccaagacaccctggggggactacagcaaattcatagcaggccacagcagtgccatgcaggtaaacgtttaccagccggttctcaacattcgctagagaagagtcgtaytttttttgcttctgtttgcaaagccaccattcgctggctccagcacacaactggttacagcatctttaaatttttcaagtgaaacacagtgatagttgacatctctcagacaccaccatgacctcatggcttgggacagtccacagtttcaacattagattatgctacatttgggggatgatattatctctgtaaagctg

SEQ ID NO: 273 - DNA, 401 bp, Homo sapiens
cccacctccccgtcaaccacccaaccaccatcaccatccaactagccatctactgttcaccacccaccacccacccacccacccacacagccggccaccacccagccagccattcacccaacagtggactcgccatgcccccaatcctcactcactcttgacccccctgccattggtctacccctcatccaccagccacaygcccagccactggccagccggccatcaaaacttcccattcatccacccaactccccagcctcctgtctgctgcccattgcccaggaacacccatcacttcactccccctgacccaacacatccaacggcctactcagcattcacccagtaaccatcatccacccagcatctcagcccatcgaccacctgatctccccctt

SEQ ID NO: 274 - DNA, 401 bp, Homo sapiens
cattttacttgaaagcaaacagaatggaaggcacacctgagcagggctgtcccccacagctactatctgagtgtcccggtcatcagccatgcacacgctctcagctgctcggcctctgcgacccagtgggaacaggtcagtgcaaggcgtggaaatgtattgctcatcagccctagcccaggctgttctgaggcaggtccracagaggagcctgtggcctggaagggcatcactctggctgaagggaacatgtccgtgagtgttcccgggtaacaccaggcagggatccccatgtgcagagactcatgtgtagagtcctcaatgactccctccaatgaccccaacaagtgtgaccagctctgaagaggctcactcctgggctgagcgggtgcagggagact

SEQ ID NO: 275 - DNA, 401 bp, Homo sapiens
aagtggagacttacggacagcatataattctccctgcaaggatgtatgataatatgtacaaagtaattccaactgaggaagctcacctgatccttagtgtccagggtttttactgggggtctgtaggacgagtatggagtacttgaataattgacctgaagtcctcagacctgaggttccctagagttcaaacagatacarcatggtccagagtcccagatgtacaaaaacagggattcatcacaaatcccatctttagcatgaagggtctggcatggcccaaggccccaagtatatcaaggcacttgggcagaacatgccaaggaatcaaatgtcatctcccaggagttattcaagggtgagccctttacttgggatgtacaggctttgagcagtgcagg

SEQ ID NO: 276 - DNA, 401 bp, Homo sapiens
gggaaacacctgcatggggcacgggggcaggtggtgggggccagagggagaggcgagggtcaggtgacaggtgagggtgaaggtgatgaggcagggatccgagtgacagtggaggggttgatgtgacagtggaagggtcagagtgacagggaagggaccagggtgatgggggagaggtcaggatgacagggaagggtcagkgggacagtggagaggtcaatgtgacagagaaggggtcaggtgacagagtaggagtcagagtgatggggaagggtgagggtgacagggtgggggtcagggtgagagaagaggtaaaagagagacagtggaggagtcaaagggaaaacggaggggtcagggtgatggggaaaggtcagggtgatggggaggggtcagggtga

SEQ ID NO: 277 - DNA, 401 bp, Homo sapiens
aaagcccccaagaaccttataaataatggcaaaaccttttaagtccgacttaaaagagtgaggagtaagatggcgcagttataccttcacagccctctgaccccaaaattcacgtgtgttcaccgggaagagccgggagagctggttatttttgtttgtttgtttgaaaacacagctcaggttcagaggttccctaacacsgagaaaggcaccggtcccgaagagggcctgcggccattgtgtcccaagcgcaggtgtcaaacgccggccgcggcgcccgcagggtccgtcccgcacgctccttctcaaacaatgactttgctgcccacgagcagagaagcctggactccagcgcagtccgcagcgcgttcgttaaggagcccaacttcggcttcttagaa

SEQ ID NO: 278 - DNA, 401 bp, Homo sapiens
gttgtttttttgtttttttgtttttttttttaaatagagatagggtttcaccatgttgcccaggctggtctcaaactcctgggctcaagcaatcctcccgcctcggcctcccaaagtgctgggattataagtgtgagccaccgtgcctggctggtagttaacaagtaaaaagaactctgttgagtcagcgggggcagagtrgactgaacagatctgaccaattcttactctcctgccaagtttccttttggaccactgcatgtctgacaattggtagaggccagatctagagtctgtggctaagggaaagttttaccctggcctgcagggaggtcaaacacacaatttcgcttcccaagtatcatattttaaattagcattctccagagtatcttccctgg

SEQ ID NO: 279 - DNA, 401 bp, Homo sapiens
ttagcctaaagccttctccttacatattttaaattcaccctaatggtttctctgtacatagtgaactgtaagctaactgcacgtgtaaacaggctgtcacctactctcgtaccaagtagccgagtctcagttaatcacagcagccagacttcaaccactcacaggcggccagctgttgaaactggactcaaataagccaarcaccatgccatcaccaatccgtctgtttctgtacctcacttccagtttctgtacatcactttcctttttctgtccataaatctttgactatgaggcagtgcaggagtctctctgaacttatttttgcctgggggctgcctgattcacacaaatggttctttgctcaatcaaactctgttaaatttaatttgtctaacgtt

SEQ ID NO: 280 - DNA, 401 bp, Homo sapiens
aacaaaagtactaaagaagcaatatatacatgtttactatttatttaatgcaggtacatatttaacttacaaaaaccagcaccccattactgtatgttgatatatatattagcttgatagaacaagaaaaaaaacggaagggaaaactagtttcaggtgaaacaaaaaagaaaacggtgtagaggctgaaatagatacagyattgcaacataataagcaattttatttctaaatggcgcctttaaatatgtcaaataaaattaattctgtttaatgaataaaaatccagtaatcgaacatattttataagcatttgggtagttgtgattattttattaagactttgatattaaactcgtgagaacacaggctttgatagagatgtttttgagaaatgcaaa

SEQ ID NO: 281 - DNA, 401 bp, Homo sapiens
gagcaagactgcccctcaaaaaacaaagagattctctcgcctcagcctcccgagtagctgggactacaggcccccgccaccacgcccagctagtttttgtatttttagtagagacagggtttcaccatattggccagactggtttcaaactcctgacctcaggtgatccacctgcctcagcctcccgaagtgctgtgattrcaggcgtgagccaccgtgcctggccttaattttttaattctataattaccacatgtaggggtgggttgcccctccacacctgtgggtgtttctcgtaaggtggaacgagagacttaggaaagaaaaagacacagagacaaagtatagagaaagaaataaggggacccggggaaccagcgttcagcatatggaggatcccg

SEQ ID NO: 282 - DNA, 401 bp, Homo sapiens
ccggctggctattatctttttgactgtagtcttctcagtgagtgtgaagtggtagctcattgtgattttgatttgcacttccctactttcctgaatgcctgatgatattgaacatctttttttttttttttttttttttttttgagacagagtttcactcttgctgcccaggctggagtgcagaggtgcaatctcagttcrctgcaacctctgcctctcaggttcaaacaattatcttgcctcagcctcccgagtagctgggattacaggtgcctgccactatgcccagctaatttttgtattttttagtagagacagggtttcatcatgttaaccaggctggtctcgaactcgtgacctcaagacatccgcccgcctcagcctcccaaagtcctgggatt

SEQ ID NO: 283 - DNA, 401 bp, Homo sapiens
ctgctatgctgatgactccttttgtaacttttctctccctgaaaaaattctggatgttttgagatgcccattttattccacaaatatttttgaggtcttgtcatgaaggcaggatggcatccgatgaattatttatatattgattcttccataacagcttagtcatgatagtaccatgttttgtaatttcaaagctgtgayggttattagggctgttcacaagaaccctgagtcacctccttccaggtacatggtgagattgttttttcatggtctcttgaagttaggcatagccttgtggcttggtttgaccaatgaaaagtgtgcagtctgatgactgtcccttccagaaagatataatcatgagtgtaagatactgcagcactcacttttctctgcta

SEQ ID NO: 284 - DNA, 401 bp, Homo sapiens
cattgaacatctttttatgtgcttcttggccagtagaggacccttaaaaaacttttaattttgacctagtttcacatgtacagaaaagttacagaatagttaaaacaaaaaactatatacccttcacccagattttccagcgttaacactttgctccatttcagctattatttcctctctctctcactctctttctctctyttttttttttttttttgagacggagtcttgctctgtcgcccaggctggagtgcagtggcgtgatcttggctcccgagttcacaccattctcctgagtagctgggactacaggagcccgccaccacgcccggcaaattttttgtatttcttttagtagagatggggtttcaccgtgttagccaggatggtcttgatctcct

SEQ ID NO: 285 - DNA, 401 bp, Homo sapiens
atttattttcttaagtgctctaacaagagtagcaagtgtgaacatctgtctcaacatgtagtcgcagactttattttcactaggaaatgtttgccttccaaatactaatgggttgaatacgtttttatttattttctgtcggcccatcacattgcaccacaaggagacagaacattttctgagcttggggacatgaaggarccagttcgcttattttgattatgtgactgtgagtgatgcagtgcaggacagctctgggaaatggacccagaggtaggagctggatgtccatactaaaggatggctctactcagccactcaagaatcagaggcagccactcacctgctccgtcaggatgatgatattgcacaggacaactggggagaatggccttgtgtgc

SEQ ID NO: 286 - DNA, 401 bp, Homo sapiens
tcttgggccgccggaaggtgggtgacgtacggatcttctttttttgtgtggctgtggacacctttcaacactgtcttcttggcctttaaatccttcgctttggtttcggctttaggaggggcaggagcttccttcttcactttcggcgccatcttgtgaaaagggaaagtttcctttctaataccattttcacttctcccraattttgtggatcgtttcttggtatctaccccagatttcaggagtgttggctggatcttagggattgtgaagtcttcatttccctgtggtgagatctgaggcatgattttaaacagtgtgagggaaggagatctccaggcactttaatagaatggagaagcaggatgggatttgagaggaaatctgattttgaaaaaagg

SEQ ID NO: 287 - DNA, 401 bp, Homo sapiens
aggaatgtcagaagtaaaggtgtcagaaaaagcaaataaaaacacaggacactcccagttaattctgaattttggataaacaatgaataacttatttgggacatatactaaataaagtatttgttgtttatctgaaactggaatttaactggacatccttacatattctgtcaagttcacagaggactgatatttgagttsaacaagaagagggtgtgtacttggtggaaaaggagggcaaagtgtttaaaggaaaccatgcgacgtgccacaaaacaaaatccaaagagcaggtgttgctgcagggggtggaatagatagaggtcaggctggagaaagggccaaggtccagtgttagaggcgttgaccccaggacaaaggatcagactcaatcctataga

SEQ ID NO: 288 - DNA, 401 bp, Homo sapiens
aggaacattgaggcagaaggaatgtcagaagtaaaggtgtcagaaaaagcaaataaaaacacaggacactcccagttaattctgaattttggataaacaatgaataacttatttgggacatatactaaataaagtatttgttgtttatctgaaactggaatttaactggacatccttacatattctgtcaagttcacagargactgatatttgagttcaacaagaagagggtgtgtacttggtggaaaaggagggcaaagtgtttaaaggaaaccatgcgacgtgccacaaaacaaaatccaaagagcaggtgttgctgcagggggtggaatagatagaggtcaggctggagaaagggccaaggtccagtgttagaggcgttgaccccaggacaaaggatc

SEQ ID NO: 289 - DNA, 401 bp, Homo sapiens
atttctcaagtatttacaaaggtaatttccaagtacacaatatgaggaagtaacaagaagaacaaaaaatgagaaaacgagttacagtttcaaacgtggagaattcacattaagaggaaaataatgtaataaacgttttagacatatccttgtaaacatcacagatcatttaaaagtttatttctggtcaatatttccacmgatatttttaggatggaaaaatactttcaacggctacgaacaagacgcctcgaaccttaggggaaaaattgcattttaaaagtgaatattgctttagcaaattcttcggctatttttcttttgtttaaatgcctttacctgtgttcattttgtatgcctaagaaacctagaaaaatagggtactgttctaagtgggaatg

SEQ ID NO: 290 - DNA, 401 bp, Homo sapiens
taccatccttgtttagtctatattaagaaaatctgtgtctttttaatattcttgtgatgttttcagagccgctgtaggtctcttcttgcatgtccacagtaatgtatttgtggtttttattttgaacgcttgcttttagagagaaaacaatatagccccctacccttttcccaatcctttgccctcaaatcagtgacccargggagggggggatttaaagggaaggagtgggcaaaacacataaaatgaatttattatatctaagctctgtagcaggattcatgtcgttctttgacagttctttctctttcctgtatatgcaataacaaggttttaaaaaaataataaagaagtgagactattagacaaagtatttatgtaattatttgataactcttgta

SEQ ID NO: 291 - DNA, 401 bp, Homo sapiens
gcaccacccccagcaccatgaggaccgtttgcaaggacagcctcaagtttttttgttgcagacaccaggattcccaagactgaggcatattttaatcgttttaaatttcttatgttttatttaaccatttgatcttgagatacttatagattgtcactctgtcactgtcagaaacaactcagatcagatcgcatgcacccraagcccggattcccgcagggatcctctcagctgctcttgggaggccgggggctgccctcacacccactgctgccctcgggccgctgggcaggacagggtgacaggcacccaccgtgcgtgggggcagagggctgagtcctctcctgtgctgggggcacccctgtccatgggtggagggttgatgagttgacgccgggtga

SEQ ID NO: 292 - DNA, 401 bp, Homo sapiens
tccttaagccatatctgctactagattgaggtttcttaatcctaacgaccatcatcaaattgcaatcatctggggagactttcccaaagcactattcctgagccccaccctagaccagttaaatcagaattgacaggaatggaacctgggctgattctaatgaatcaggcaaaataaatgctaagacccacatgacagacrggctttctaaagcagacctttagaatcagatgaaataaaccttctcctctgaaagctcccccaccaattattccaggccacatgcttcactgctcaggtaatgagagggtctgggctccagaggttcagccactgctctctcaagatacttttctaaaacgacaaacctcccaaagaattttccagagtttcacatgtgc

SEQ ID NO: 293 - DNA, 401 bp, Homo sapiens
caaagtttcccagctgttgacatacacaagtttgtttggtgcaacctgtcagatgcatcccttagacaggccctttgatactctgggaaagacattggacttacagtcggaacgaaaagaaagaaatgtgatatgtatagcgtgcagtgagttggagttttacctgtattgttttaatttcaacaagcctgaggactagcyacaaatgtacccagtttacaaatgaggaaacaggtgcaaaaaggttgttacctgtcaaaggtcgtatgtggcagagccaagatttgagcccagttatgtctgatgaacttagcctatgctctttaaacttctgaatgctgaccattgaggatatctaaacttagatcaattgcattttccctccaagactatttacttat

SEQ ID NO: 294 - DNA, 401 bp, Homo sapiens
ccgccagagagcgtaggcgagggtgaaggagtccgggagaactcttggaaagagactactttatgggtagagggagtagccaaagtgaagctcccccagcagctgggctgcaccggcgcccagcctggccacgccactggggagctggacccctggggctcctcactctcctgtttaaacccggatgtcaaggcgcctccrccttgggggaggatggtgaggcagaatggtgaggcactaggatggtgaggcactaggatggtgaggcactaggatggtgaggcagtgaaagcaaagcttgacctgcagctgggtggacacttgcagccgagggtccccccggcccactcatttctggtttattcgtagttccagaacatctgtggttccctgggccgggg

SEQ ID NO: 295 - DNA, 401 bp, Homo sapiens
gccccaccgttgcctgctctccacagaggccccactgcccaccccacactcctggttccaccggggactcactccgtccacacatatcctcagcgtgggcactggtcccttgtccaggaccagatgctgagcccaccctccagctggggctcttgtgaccccagtgaagcccacggagccactccctgcagtgaaacctgragcacatctcgatgacaccgacgtcactgaccgggaaaggccagctccagaacataccatgcgtgaaggagccaccatgggccagggtggtggctctgtggacacaggccctgggctgaggccaccctcttctgtgggggtcagcacggggaccggcccttccctcttggacctggtggaccctgtggcacgtggggtgg

SEQ ID NO: 296 - DNA, 401 bp, Homo sapiens
ctgtctttgagttatcattgaaatgtctaggtagagacacttagttctttctcttgttcctggaactcaagagaaagatctgtccagacagagactgattttgaagccaatatattattaggtaatgaaataaggagaccaagaagagagaggaactgaagatgtaaatccagaattaaagcatgaattaaagaatgggcwaaaagaaagaacatgttggtaaagtgcagtggctcatgcctgtaatcccagcacttcaagaggccaaggcaggaggatcacttgagttcaggagttcaagacctgcctggccaacatggtgaaatcccatctctactaaaaatacaaaaattaactggggatggtggtgtgcacctgtagtcccagctactgggaaggcc

SEQ ID NO: 297 - DNA, 401 bp, Homo sapiens
gtagaggccagtgctacttctaaacatcctacagtgcgcaggacagccccccacaacagagaatcacccagtttgaaatgtcaccagtgccaaggctaaaaaaccttgggctgagggaacagcatgtgcaaagatgttgagtaggagaaaagaagtatggtgacttgggcagcatagtttgattttaaagaaggccgggcrcgatggctcacgcctgtaatcccagtactttgggagactgaggcgggtgggtcacttgagacctggagtttgagaccagcctggccaacatggcgaaaccctatctctactaaaaatacaaaaaaatagcctgttgtggtggcaggcgcctgtaaacccagctgcttgggaggctgagacctgagaattgcttgaaccca

SEQ ID NO: 298 - DNA, 401 bp, Homo sapiens
gatcatgccactgtactccaacctggatgacagagcaagattcagtctcagaaaataaataaataaataaataaataaatgtcagttgaattaaatagatggagattggtgaaattggaaattggcataggagatgaagaaattttctagaacgctcaagagggaaaaaatgctaagtaaggctggacatggtggctcacrcttgtaatcccaggactttaggaggctgaggcgggtggattgcttgagctgaggagtttaagacaagcctgggcaatatggcaaaaccctgtctctaccagaaatacaaaaaaaaaaaaaaatagccaagcaaggtggcacaagcctatagtcccagctacttgggaggctgaggtaggagaattgcttgagcccagggg

SEQ ID NO: 299 - DNA, 401 bp, Homo sapiens
gtcccagctacttgggaggctgaggtaggagaattgcttgagcccagggggtagaagttgcagtgagctgagattgtatgcctctgcactccagcctgggtgacagagccagaccctgcctaaaaaaaaaaaaaaaaaaaaaaaaaaaaaaaaaaaaaaaaaaaaaaaaaacctctgtctttgttggaatttccaaaacgrgaaggaagtaggaactattgacagagcaggtgctttgcaaggatatttgtcccttgctctttacagcacatctgcctgtaaggatgtgggtatcattattccacttttattttatgaggaattaagacccagaaaagttaagccacctggccaaaggacactcaaaaagtaggtggcaaagccaggactccagtcccatt

SEQ ID NO: 300 - DNA, 401 bp, Homo sapiens
cgtaaatgagtatgtgactcagccttccagaagtattggaaaagccccatgcactttcctatggcaccagcagatggcggcaaaagagcagcttagcaaaggcagaggcaaacacttggcgtttctttcatttatgtatcctctcttccttgatctttgcaaaacaggtagcttgcatttcccccatcctccaccttcacyactcccctccccccattctgggaggaaccaagaggtagtgctcacagactgcagtcccctctttctgttttgacctctttgccccacctcagagacaaggttatttcaagaaatggaagaagaaataacaccagaagaactttaatggcaatgaaaaccagtgcagaaatgacctttttattgacaaatagagaaaaggt

SEQ ID NO: 301 - DNA, 401 bp, Homo sapiens
cggcgagattgcgaccggcgccaggtgagtaataggcagcaaaatccagagagactgcgagccgttcatcccctccaccccccgcccatcagcgctacaggagttactcattaacatcacaatatacaaatgagatacacaggagattaaaaaaaaaaaaaaaaaaaaaagaggaacgccagtaagagaggctggacacamgtctgagcagggtccccaaatcgggggcggggggagagaagggaggaggtaagcaaatttggactgagaaagaagagaaaagcaaagcatttcttcccttttggaggctgagttcttttttccccaattcgggagagcgcaaccagccaactaccaccttctcagaggcgcggacaccgcgcgttgtcgctggatccaga

SEQ ID NO: 302 - DNA, 401 bp, Homo sapiens
gccatgtacgtgtaggggtaagggaacaggcttccgaaaggggacatggccaggccctggggtgagaaaagagacacacagcgagtcagccgggtgggaggagcttatctcatccctctcctctcttctccccctccaacattgggcaagccatgaatccctggtatgttctgttgttgttgttgttgtgttttttttttkggggggggagataggtgggggcaggagagaagtcaacactcagaaatctctaggtacaaatcttagctgtgtcaccctaggccaagagactttgattaacttgtgcctcagcttccttataggaagaaaaaacagggaaatcataagtaattcatatacttattgaggactttgtagatattaaatgagtgaaagcctgc

SEQ ID NO: 303 - DNA, 401 bp, Homo sapiens
aaggtaataaaaaccagaccagctactcaggatttctggctaggggagaaaagctggtctctactaggattctcctcccaggatcccaaatggcttttgccttaaatttttagctggttccttttgaatgtgacttcttgatccttcttccccaaggggaagaagatgaaccttaaaacagacaggacaaagcccaagagkgggggaggagtgggtggcatcaatgacagcagttctaggagtttctctgtggcaaaattagtatggcgtttttttgctcagcattgttcctacctagtcaaggtgtttttctgtctcctcgaggtgtcattgtcctttccccccaaatgctccacaaatcacaaaagacaactcaagactctcgtctccactctcttgta

SEQ ID NO: 304 - DNA, 401 bp, Homo sapiens
tgcaaatacgtaagctggacgcgcctcggtgcggatccaggcctcgctgagcacggaaccggagaagcagctcacggcctcggattctactctcacccaagggtggaagggggtgacagggctcggatcttgccccaaacgctgcacaggcccgcaggagagcaaagaggtctcggacaggctgtccttccccagctctcsgtcgcgacaaggtcccgacaggcccgggaaccggcgaaccgcctgcaggtaaccctgcgctgggtacctgccttccctctccagctaccggggcggggcgcggcgcggcgcggcgcggcgcggcgggctgcccgggtgggggatgaagaaagcacggcggacccagggcagcgaagcgcaggtctttttccttttgagaa

SEQ ID NO: 305 - DNA, 401 bp, Homo sapiens
cacatacatatacacacatacatatacacacatacacatgcgcaaacacacatacatacacacatatagatacacacatacacacatgcacacatgcatgtgcacacacatgcacacgtgtgcacacacatacagacaccacacacataccgcacaccacacacatacacacatacatgtatgcacacacatacacacacmtatgcatacacatatacacacatgcacacacacacccctgcacatacatacacatgtgtgcacacacacacgcacacacacacagcacatacacaccacacacagctatttgtttgaggaaaagcatggtttatttagatggtgctttgcataactttctaaattgagttcaaattggcataagaccatgtattttgtgc

SEQ ID NO: 306 - DNA, 401 bp, Homo sapiens
gtgagtctctttctatcctccctcctttgcccaatgtctaccttcccaagagtctccagtatctaggacattgtcaaaaacatggcagacactcaagaaatgttccaatgactaattcaatcatgcaaaaaatggcgataattccactcatgtaaatgatcagactggatatagatggaggctgtcccatagagccctgtrctttcttcctaggctcttccaaaatacatgggtgccatggtgtctttgtctggagctgggttccttaagcaactttacggcctctttagtactctggaagcgtgttctacattaagggttatcggctctctggctgcattctgcctggacaatccatgctggaaatgccctacagctggagactgaaattcagagtccgc

SEQ ID NO: 307 - DNA, 401 bp, Homo sapiens
acttacaatttatgagtcttcttttgtcttcattagactaggtaaagttttgtcaattttgtctatcttaaaaatccaattcctaattttattgatcttttctattcagacactttgattcttctcaagccattagagatgtagccttccttaaatcccaagttgttagcagggcctgaaaggtctcaaaggatcaggtcytactgctttcatccctccccattctccccttgtttactctgttccagccaaaattgcctccttgttattcctgtcactagaagcacactcccatttcctgtcccctctgcggggaacacttctccccatgatacatgcatggctcacattctcacttcactcaagtctctgctcaaatgtcacctcatcaaagagaatag

SEQ ID NO: 308 - DNA, 401 bp, Homo sapiens
ttacaatttatgagtcttcttttgtcttcattagactaggtaaagttttgtcaattttgtctatcttaaaaatccaattcctaattttattgatcttttctattcagacactttgattcttctcaagccattagagatgtagccttccttaaatcccaagttgttagcagggcctgaaaggtctcaaaggatcaggtcctmctgctttcatccctccccattctccccttgtttactctgttccagccaaaattgcctccttgttattcctgtcactagaagcacactcccatttcctgtcccctctgcggggaacacttctccccatgatacatgcatggctcacattctcacttcactcaagtctctgctcaaatgtcacctcatcaaagagaatagta

SEQ ID NO: 309 - DNA, 401 bp, Homo sapiens
acttttcttccccttgacaggctgtcagatattaagaatcagacttcttcctgaatagtgacttcaaggatgggtactatactgaaaatggaatgtctcagattggctttccagaaaagtgcctgagataaagacaagaatatatcaaggtttctgggaagacaagtaatgaagtacaggaagcaggagaagggaaacagraggaagacaaacaagggggcaacttcaggcaaagcctcaccaaaccctagagctaagcaaagcttcggagctgtcccatccctagtgaaggctgttggactttcatattctcaccctgagagtcgttggtcaagggctgcctggtgttgggcgagggccaatgtaaactcccaggaattgcaactctgcacatatcagca

SEQ ID NO: 310 - DNA, 401 bp, Homo sapiens
aacaaagtactgggattacaggcatgagccatcacgccgggcccagtttctgttttttgtttgtttgtttttgagacagagtcttgcactgtcacctgggctggagtgcaatggcacgatctcagctgactgtaacctctgcctcccggattcaagcaattctcctgccttagcttcctgagtagctaggattacaggtgyccgccaccatgcgcagctaatttttttttgcatttttagtaaagacgagatttcactgtgttgtccaggctagtctcgaactcctgaccttgtgatccgcccacctcggcctcccaaagtgctgggattacaggcatgagccaccacgcccggccccagcttctgttttactgaaaaaagcaagttacacacagtagact

SEQ ID NO: 311 - DNA, 401 bp, Homo sapiens
ctctcactatgcatcatcttccctcttgacaaccatccagaatattctcctctgacccatacactggagcaggggttgacaaactttttctataatacactagacagaaagtattttcattttggaggtcacacagtctgtctcaacaactgagctctgccattgtagtgcaaaagcagccagagacaatatgtaaacaartgggcatggctgtgttccaataaaactttatttacaaaagctggcagtgggccagattggcccataggctgtagtctgccaacccctacattagactattacctccctctgtatcaaaaacccacctgcccatgtcccaggctgatagggatctgctttccaagcccagtgataacacgaggccctttttaccttcctga

SEQ ID NO: 312 - DNA, 401 bp, Homo sapiens
tgatttggtcctctagggacacagatccccccacgctcccttggaatccctattctaaatagttgatattggcaggatcccagtgtcttaaataatcactgaattttagagtgggaaagaatccagaatacactctcccacccacccacccatttaagaagtccaaagactgtcttctttaagaactctgagggtcaaagrcacacagaagatggagcagcagttgtcttgggcagtggtgtgccagcttgtgtttggggcaacttgtggcatgaaaatgacgtgtcttcgcacaacaaagcggaaaatagcagtaagtgttccgggtaattcaaacaccagctgtctctgtggtttcggagctgtggttcgcttcttaatcaaaaggcattcggcctaca

SEQ ID NO: 313 - DNA, 401 bp, Homo sapiens
tatcagcttcaagacaaaatataataaccccaaattagctcttcctcatgagcaataaattataaataagttgaagttcacagccagagaacttgagacaaccctgatacagccttaaaaaaaaaaaaatcacatctcaggttacaatataaaaggcctgtctggctttttactcaatattatagttgtcataatggcggkacatgttcagaaactacctcctagtgttttaggaaacatttcacgctactctaattcagagcatggccatctgaaaatgttctccctgccctggaaagaggcaggctgatgggggctttaatgccaactatttgcacttcttacacccatgaaaatatatgaacacccttacttctaagctaccttttgattattcctag

SEQ ID NO: 314 - DNA, 401 bp, Homo sapiens
attttattattattatattttaagttttagggtacatgtgcacaacgtgcaggtttgttacatatgtatacatgtgccatgttggtgtgctgcacccattaactcgtcatttagcattaggtatatctcctaatgctatccctcccccctcccccgaccccaaaacagtccccggtgtgtgatgttcccttcctgtgtccrtgtgttctcattgttcaattcccacctatgagtgagaacatgcggtgtttggttttttgtccttgctagaagacatttatgcagccaaaaaacacatgaaaaaatgctcatcatcactggtcatcagagaaatgcaaatcaaaaccacaatgagataccatctcacacccattagaatggcgatcattaaaaagtcagga

SEQ ID NO: 315 - DNA, 401 bp, Homo sapiens
gtgtgctgcacccattaactcgtcatttagcattaggtatatctcctaatgctatccctcccccctcccccgaccccaaaacagtccccggtgtgtgatgttcccttcctgtgtccatgtgttctcattgttcaattcccacctatgagtgagaacatgcggtgtttggttttttgtccttgctagaagacatttatgcakccaaaaaacacatgaaaaaatgctcatcatcactggtcatcagagaaatgcaaatcaaaaccacaatgagataccatctcacacccattagaatggcgatcattaaaaagtcaggaaacaacaggtgctggagaggatgtggagaaataggaacacttttacactgttggtgggactgtaaactagttcaaccattgtgg

SEQ ID NO: 316 - DNA, 401 bp, Homo sapiens
tcattaaaaagtcaggaaacaacaggtgctggagaggatgtggagaaataggaacacttttacactgttggtgggactgtaaactagttcaaccattgtggaagtcagtgtggcgattcctcagggatctagaactagaaataccatttgacccagccatcccattactgggtatatacccaaaggattataaatcatgcwgctataaagacacaggcacacgtatgtttattgcggcactattcacgatagcaaagacatggaaacttttcttgaagttgcatctgcacagcaaacatactgttctttttaagttagagattttgttactagcaagtaaaaatagagatggctttattcatgttttacataaaatagagaaaatatcagttttccatgtt

SEQ ID NO: 317 - DNA, 401 bp, Homo sapiens
actcttatttttacttggggatagctttgggaaggggtaatagagaatatggccatcaatcccccctgcaaagctaccccaagttaccaccttaagagcagaatagtttattttaaaataccatatttcatcaaacataaaatgctattaattcaaagatgtatttttatgttaaagagaagaagaaagaaagagaaagaragaaagagaaagagaaagagaggaaggaagaaaggaagaaaggagggaaggaaggaaggagggaaggaagaaaggaaagaaggaagattatgaatggttaagacaccactgactataagaagcatcatgatttcaagaatattgaggtggaaaaaatacatatatcagaattggtgaaatatgttaggttttcaaatgac

SEQ ID NO: 318 - DNA, 401 bp, Homo sapiens
atgtaatctatacctcaataaaaaagattaagaatgaaaaaactgaatgtaaaacagacatttctaagatattaagagaatatcaaattcatactcatagtagactataaaagagaccataattgagatgttattacacctttctcagtaatagaacaaccagaccccccaccaaaaaaaagggtaagggcatagatattracaaaataattaactaacttgactatagatatagagatctggagaatacctatgtatcaattgacataaatagaacaccaaactccaaaatgcaaccctgtactacagtgaaaagttactgccacacacaattgcatgaatgaatttcaccaccagaatgaggcatacaacaaagaatacatcctgtatgaatccaatta

SEQ ID NO: 319 - DNA, 401 bp, Homo sapiens
tgtgaaccaggatcaacactgacagccagtcgcattctcaacatctggtttacttccagctcaaaatgggaactttccattgcctctggtcacttgaatcctctagtccccaaaggaactcaactaaccaggcatgctaattggatcgagtcaggaaccaaggtttcaattagatcagggcgctcctcttcatgacagccygaaacaggcattcccatggggtgaatttagtcttaggccaggcatccaggggaaaacaagtccttaatttgccaaagccagtccaggatccagttggattttcattcagacacatgcctggattgccatactgaacactccctggcctaatggacataattcatggtctgcacccctcccaggcagtgcagaaacaaaac

SEQ ID NO: 320 - DNA, 401 bp, Homo sapiens
gtccccaaaggaactcaactaaccaggcatgctaattggatcgagtcaggaaccaaggtttcaattagatcagggcgctcctcttcatgacagcccgaaacaggcattcccatggggtgaatttagtcttaggccaggcatccaggggaaaacaagtccttaatttgccaaagccagtccaggatccagttggattttcaytcagacacatgcctggattgccatactgaacactccctggcctaatggacataattcatggtctgcacccctcccaggcagtgcagaaacaaaacaacgttgtcctcacctggctgtctgggcccttcaaatatttctctgctgacaataatcacaagtcagcctttgaccaggccacacttcacatccttgacctagaa

SEQ ID NO: 321 - DNA, 401 bp, Homo sapiens
gaatatatgaatcaaagaagttataaagcacaggcttttgggatttaaaagaaaatcaaagacagctgggcacagtggctcactcctataatccaagcactttgggaggccaaggcgggcagatcacgaggtcaggagattgagaccatcctggctaacatggtgaaaccacatctctactaaaaatacaaaaaattagcygggcatggtggtggatgcctgtagtcccagctacttgggaggctgaggcaggagaatagcgtgaacccgggagacggagcttgcagtgagccgagatcgcgccactgtactccagcctgggtgacagagggagactctgtctcaaaaaaataaaaaataaaaataaaaataaagaaaagaaaagaaaaaataaaagaaaa

SEQ ID NO: 322 - DNA, 401 bp, Homo sapiens
agggagactctgtctcaaaaaaataaaaaataaaaataaaaataaagaaaagaaaagaaaaaataaaagaaaatcaaagattataaatatagtaatatttgggagtttatttttttctctaaatcctgtcagagagaaagcagttctcatttacttaaatacacttcaagctgaatcactcatgaaatgtcattttatttraaatatgtcaatgatacttgaataaacacatccacaaccactaaattcatcaaatgaaaacaatggactgggaaagttcatttctatcacaggaaaatttcaagcagttatgaagcccagatatttcaatgatcttgggatgattgtagttaaatttctttgtgtggctgcgcctaaacccagacgggagaaacaagtgc

SEQ ID NO: 323 - DNA, 401 bp, Homo sapiens
aaataaaaaataaaaataaaaataaagaaaagaaaagaaaaaataaaagaaaatcaaagattataaatatagtaatatttgggagtttatttttttctctaaatcctgtcagagagaaagcagttctcatttacttaaatacacttcaagctgaatcactcatgaaatgtcattttatttaaaatatgtcaatgatacttraataaacacatccacaaccactaaattcatcaaatgaaaacaatggactgggaaagttcatttctatcacaggaaaatttcaagcagttatgaagcccagatatttcaatgatcttgggatgattgtagttaaatttctttgtgtggctgcgcctaaacccagacgggagaaacaagtgcaaactggctgagttcattgc

SEQ ID NO: 324 - DNA, 401 bp, Homo sapiens
aatgtcattttatttaaaatatgtcaatgatacttgaataaacacatccacaaccactaaattcatcaaatgaaaacaatggactgggaaagttcatttctatcacaggaaaatttcaagcagttatgaagcccagatatttcaatgatcttgggatgattgtagttaaatttctttgtgtggctgcgcctaaacccagamgggagaaacaagtgcaaactggctgagttcattgctgtccatataaggtgtattccaaacacgtctttctatgccagcacattggcagaacattaaatctttgtatgcaaaacaaagaaggccattaaagtaattaattatatatagattatgtaattttttaaaatcccatttgccccaattattggctcaattgagaa

SEQ ID NO: 325 - DNA, 401 bp, Homo sapiens
tttattttctgtcggcccatcacattgcaccacaaggagacagaacattttctgagcttggggacatgaaggaaccagttcgcttattttgattatgtgactgtgagtgatgcagtgcaggacagctctgggaaatggacccagaggtaggagctggatgtccatactaaaggatggctctactcagccactcaagaatcmgaggcagccactcacctgctccgtcaggatgatgatattgcacaggacaactggggagaatggccttgtgtgcattttctccgcaggaggctcagcagcatcatgcctggattggtggtaaattttgctaagtccacttacaacattacaggctgaaatcctaactttatggaacaatgccagtggttctgttgtttcta

SEQ ID NO: 326 - DNA, 401 bp, Homo sapiens
attgcaccacaaggagacagaacattttctgagcttggggacatgaaggaaccagttcgcttattttgattatgtgactgtgagtgatgcagtgcaggacagctctgggaaatggacccagaggtaggagctggatgtccatactaaaggatggctctactcagccactcaagaatcagaggcagccactcacctgctccrtcaggatgatgatattgcacaggacaactggggagaatggccttgtgtgcattttctccgcaggaggctcagcagcatcatgcctggattggtggtaaattttgctaagtccacttacaacattacaggctgaaatcctaactttatggaacaatgccagtggttctgttgtttctatacagaattgaaatactatcaat

SEQ ID NO: 327 - DNA, 401 bp, Homo sapiens
ggagatgaaagatgaatttagttttatttgtttatgtctgccttccttcactagactgtaagattctctagaccaggagttggcaaatattttctataaagagccagatagtaaacatattaggctttgtgggccaagagaccacatactctgctaccgtagcatggaagcaagcacacacagtatgcaaagcaatgaacrtggcagtgtccccggccagcacaaaccatgcagtggactggatctgggcaagggtcataagttgccaacccctgctatagatcagggacctgatcgtatttgtcttggtacctcacacctagcacagcacttggcagatagcacctgattgagaaatgttgagtgggtgagtgactgggcagcattcttgcctcaggaat

SEQ ID NO: 328 - DNA, 401 bp, Homo sapiens
gaaaaaatcaataagagttcgtgactaactagatggtaaccagagagcagagaaggaaagaaaggcattctacccaattatgtttttagattaggagatttactgatgccactggccaagctagaaaatacaaaaggcagctccagtttgtgacgagacagaaatggcaagttggtttgggaaactgtttttttttttttycagggctttgcaattagaagatagtaagtagctgaaaatgtttttctgaaacttgcaagagagatagaggggaaccatcaccctaggtgcggtcccaaaactcatgtaagtggacgagaccccatcccggggacagaacatacaagataaaggggccaggagaagagctagtaagaaccccagtatttatggattaatca

SEQ ID NO: 329 - DNA, 401 bp, Homo sapiens
agcaaaaaagattgcgagaggagaaagggtggtttaaaaaaaataaaaggtgacaatgttgtcctggaacttaaccaaggaagaagtgtggaggtaaacagaaagagaaacaagaaaattgaacaaggacattggcttgggtccagcagtcacgagtgactgtcttaggctagtgtccgggcggtggtagagaccaaagasgttgagctgcgcattctcagccctatcctgttcaacattttgattagtgttacaaatgaagacattgcttcatttattcaacaaatgttcattgagcacatattctgtgccagacacaacctggaagaaaagcttagcaaaccaatggatgagataaagctggaaggggtcgagattaatcctcaggcaggggagctaaa

SEQ ID NO: 330 - DNA, 401 bp, Homo sapiens
aggaagaagtgtggaggtaaacagaaagagaaacaagaaaattgaacaaggacattggcttgggtccagcagtcacgagtgactgtcttaggctagtgtccgggcggtggtagagaccaaagaggttgagctgcgcattctcagccctatcctgttcaacattttgattagtgttacaaatgaagacattgcttcatttaytcaacaaatgttcattgagcacatattctgtgccagacacaacctggaagaaaagcttagcaaaccaatggatgagataaagctggaaggggtcgagattaatcctcaggcaggggagctaaacccatcagaattttttataaacagagctagggccaacttagcatattgtcaagccaaagtgattgttttgtggccca

SEQ ID NO: 331 - DNA, 401 bp, Homo sapiens
agtaattatttttaacgacaagtagtttaataactctcaagtagtaaaataattcatactttgctaacatcactgccacatagagctcctttagtaaatgctttatggaaaaatgtaatctgatttgtgactaatatatcaattggctagcaataaattttgtttctatgctgtggaagcatttgctttctttagtagatkaagcaatctccttgcaatattatttacggtattaatttgctttttagtcaagtatccagaaaaaagcaagagctgtagagtcagactgacataggttcaaatcctagcctagctgctcaccagcgatgtgactcagggaggatggctcaatttttgagtcatcagaaatatcttacgtgataaaatgggtataatcctat

SEQ ID NO: 332 - DNA, 401 bp, Homo sapiens
ccacctggagcatggatgggtcatgaaaagcacaggataggatgtcttgccaggtgtcacggtcaaggtcacaggctggcctctccacagggatgtgctgagtgctgccggaggagatgaggtcaaccctgcttccttctgcagccaaagctcttctcccagatggacctgaggaagcctactgtccacatcccagggagkatgctgctccctttgaaggagctggggaggcttccagcagccaagccccccaagaccaatgaaatagccttggctccagtttcaaaatcacagcagcaagagagcagacatccacgggcatcacagggccaaacctgtctcatcaaagacattgtgcaccctttcctagttaagaaaataggaatatcagggggaaaaat

SEQ ID NO: 333 - DNA, 401 bp, Homo sapiens
attgtgcaccctttcctagttaagaaaataggaatatcagggggaaaaataaatagtatgctttctgttgttgtttcaaagggaggatctttgaggcattttaacatcaattttttgggtgtttattttcctaatcatcatggcatatggattgggcatctagtaagtgcaaggtatggtgctaggctccatggaaaagasagagagaaaaaaaagggcatgccctgtactcaagagacttctcatctagttaggagatggaagaaacacacaaaaagagtttaacaaagacccaaaaatactgaggggatagacatggcgtaggcattccaagtacttgttatcaatccagtatctatctgggtagaaaaagaaaatgaactgggcttcattttctcagg

SEQ ID NO: 334 - DNA, 401 bp, Homo sapiens
ttagttctagatgttttggagaagaggaaacaaatccaggttgtcatactctgaaggctcttgaatttggaagagtctgtggaggaggtcagagggggaacagaaagcaactcataagctggaaacacagcctaaaattatcattgctttgcttttagtcacaatagttttttgctggtttttttttgtttttttgttttkttttgtttttttgttttgaccaatgataaaagaggacatgcgtttctactgcatgaagtaggcatggattttaaaaggcagaggaattattgcactcaggaactctttgaggcagaaagaaagtcattactgtgcctgcaggaagagctgggcattcagtacacaatgagtgaatgaatgagtgaatgaatgaatgaatg

SEQ ID NO: 335 - DNA, 401 bp, Homo sapiens
ggaaacacagcctaaaattatcattgctttgcttttagtcacaatagttttttgctggtttttttttgtttttttgttttgttttgtttttttgttttgaccaatgataaaagaggacatgcgtttctactgcatgaagtaggcatggattttaaaaggcagaggaattattgcactcaggaactctttgaggcagaaagwaagtcattactgtgcctgcaggaagagctgggcattcagtacacaatgagtgaatgaatgagtgaatgaatgaatgaatgaatgaacaaatgggtaagtaaatgcggcgctcagagcgctgatgtcctgtgcgctcaggtatccaggcagagagaaaggggcacctgggagatgggattctatgactgagtttgcctctg

SEQ ID NO: 336 - DNA, 401 bp, Homo sapiens
cattaaccacaagggaacgagcccagagttgaattcagataaacctgaatgcaattcccagtctgatcatcgctggttacaagaactcaccttaataattgaatctcatggccaggtgtgatggctcatgcctgtaaccccagcactttgggaggctgagacgggcagatcacctgaggttgggagttcgagaccatcctkaccaacatggagaaaccccgtctctacttaaaaaatacaaaattagctgggcatggtggcgcatgcctataatcccagctactcgggaggctgaggcaggagaattgcttgaacccaggaggcggaggttacggtgagcagagattacaccattgtactccagcctgggcaacaagagtgaaactccatcacaaaaaata

SEQ ID NO: 337 - DNA, 401 bp, Homo sapiens
agaaaccccgtctctacttaaaaaatacaaaattagctgggcatggtggcgcatgcctataatcccagctactcgggaggctgaggcaggagaattgcttgaacccaggaggcggaggttacggtgagcagagattacaccattgtactccagcctgggcaacaagagtgaaactccatcacaaaaaataataataataayaataataattgaatctcactaaaacagtatacttctcatttgtgtaaatagaaatgagcacttgctccctggaattattttgacccttataatggagttgcattttcagctggggtgggggaattaggttgtaaagttaacgtatatactcatattttccttgaaaatgcactaaatggccaaccactgtccaatagaaa

SEQ ID NO: 338 - DNA, 401 bp, Homo sapiens
aagttaggcacttagaattacaggaagattcatatgcggatcatgccgaatctttcagaacattagcacccttctaacatttcacagatactcttaaaaactgaagagtactgcagtgaacagtttattataaagaaccatccacattcatgtatacaccatggaatactatgcagccatgaaaaataatgagtccatgtmctttgcagggacatggatgaagctggaaaccatcatcctcagcaaagtaacacaggaacagaaaaccaaacaccacatgttctcactcataagtgggagttaaacagtgagaacacatggacacagataggggaacaacacacaccggggcctgtcagggggtggggggaaggagagaaagagcattaggacaaatacct

SEQ ID NO: 339 - DNA, 401 bp, Homo sapiens
cctaatacatgctatggccatggatgaacccctaagacataatattaagtgaaagaagtcagttaagaaaggccatctactatatggtcccatttatacgagctgtccagaaagacatgggttcaagtataaataattttcctaaaatataattcactcctctaccttatactgaagtttaattaaaaccacacctaacaycgatttttaaatttacttcttaaaatatattacttgcttgtatcaaaatgacaattgaaaacatatttaacagaatccaccaaacaaaagaccactatgccacattgatatccaataggaaattccaaatattgcaaccaaaaatacaaatcaactctttttaaagagataatagtaccgttagcctctgtcttctaaat

SEQ ID NO: 340 - DNA, 401 bp, Homo sapiens
atatatttactatacttttttagaataggttttctactcatttaaaaaaaattacctataaagcagcctcaggcaggtccttcaggaggtatttgagaaaaggcattgttatcacaggagacgacagttccatgcgtgttactccacctgaaggctttccaatgggacgagatatggaggtggaagagagtgatactgatkatcctgaccctatgtaggcctaagctaaggtgtacatttgtatctttgtttttaagaaaaaagttcaaaaggtgaaaaaaattaatagaaaaaaccttataggataagaatataaagaatactgtatatgttggtacagcggcacaatgcatatattttaagctaagtgtcataacaaaaaagtaaaaaatttaaaaaaa

SEQ ID NO: 341 - DNA, 401 bp, Homo sapiens
actgatgatcctgaccctatgtaggcctaagctaaggtgtacatttgtatctttgtttttaagaaaaaagttcaaaaggtgaaaaaaattaatagaaaaaaccttataggataagaatataaagaatactgtatatgttggtacagcggcacaatgcatatattttaagctaagtgtcataacaaaaaagtaaaaaatttwaaaaaattaagtttaaaaagtaaaaaattaactttagcttattgtaagaaatacaaatgttttgataagtttagtgtagcctaagtgtacagtgtttataaagtctacagtagtacacagtaatgctgtaggcgttcacatttactcatcagtcactcatcaactcacctcaagcagtttctagtactgcaagttccctt

SEQ ID NO: 342 - DNA, 401 bp, Homo sapiens
aaacacatgtgtccttttgtttcacggtctcacttggcaccaagcacctagttcatttgcccatcagattagctcgaattacatgcttttactgctcctacaagctaatgcaagagacctacaattaactgtgggctggtttaagccctaaatgaaactaatgtctgccccgaccaaatgcctcaggacccactttttttycctgtaaattgctatttttgtaggctgtcttccagcagttgagccccaaaacaagttggaactgtaaacctactagattctactcacaagtattgttgaatccgtctcttgcactgtctctgaaagcttagcaaaatgcacaatagtgtccttatgaaggaggacagaattgccactcttaatatagtctcattcaagtt

SEQ ID NO: 343 - DNA, 401 bp, Homo sapiens
cccatgaggcactgggagcacctgatgcaaattaaggactaaaaccacagtcttgggttaaataatgtcactgtggtatagaatgatgacagggcaagcttggatcaatgtcttcaggagcactatggaaattacaaattttattctgaaatctatccagctataccacgagggctaaagataatcatttggcctctaccycttcctgccttcaaagtaggaggcagagtttctgtggcagagataatacggcatgctcaccaaacctgcgtcttcttcctggccacccaggaagcctgcattcccaacatcctttgcagtttggttgagaccgcatgaattttagtgccattttattcactccccaccccacttccctctttcaggccataggcatgggc

SEQ ID NO: 344 - DNA, 401 bp, Homo sapiens
tcaaacaggacaagagaatcgaaggattatgtccagcatggaatttcagtccccttcccttttgacctcaactgacaaggccctttgtgccttttatgtaatattagtggttttcagaatgtgctccctggaccagcagtgacagcagcatcacctgggaagctgttagacatgcagattctctgagcccatcctacccaygcccaagtgaaccagaaactctggggagactgggtgctcaatgcaagatttaatacattctcccagtgattctgatgtttgccaaaatatgagaaccattgctttatgttaattgacttactttgttatttcacgcccactatctataagctaattcccactgcagtgaggcagagagagatggctccacactgcactct

SEQ ID NO: 345 - DNA, 401 bp, Homo sapiens
tctggggagactgggtgctcaatgcaagatttaatacattctcccagtgattctgatgtttgccaaaatatgagaaccattgctttatgttaattgacttactttgttatttcacgcccactatctataagctaattcccactgcagtgaggcagagagagatggctccacactgcactcttaggtacaatcgagcgtccscatttggtgttgagaggtctgctttattcttcccatttatttgatgggcacgtttttaacttagaacaatatttctctccaaacagtgtgagcatcttattgtaagtttttacaaacatttttttagccatttcaggtaacaacccccagcgtaacaatcccccacgtggcctgtaacacccagaactctggtgaatgta

SEQ ID NO: 346 - DNA, 401 bp, Homo sapiens
ttcaggggaatcagcgtgtttgtgttggcttcctggaccttcgaggctcacagacctagccagctcaggcacagatcagcagccggggaaggcattagtctcccccacatatctaccctgcgagctttatggccaagagcaatgccagggagttcaagtgattagttatgttagggaaacgaaagccaacaacccaagaawcagcgtccccaggaaaaggtcaataggcctcggtgctgtaatgaatgtaacaaatatttaaataaggataatattgaaatgtgttcagtcatcttgatttatttccaattacttggcttttgctccactgagtagttctgaaattcaaagttgatttctgctgacttgtggagccacaccacctctatttttcttccttc

SEQ ID NO: 347 - DNA, 401 bp, Homo sapiens
tttttcttccttctacgtgcctttcaatcctatttcacagtcccttccaaaaggaccacaaagcatggcaaacagagatagactcagtctaccaagtggctacaaaggatgttgttttataaagcagagaacctgagccaagcttctgcacaatacagaggaagagtcaaggctctagacagactggtccaggccatggtraatggcgaggcccattcaatcccaaccatcagaggagcttttttcctttccaaagcatgagtcctacagggatgaagatgagaatctccttgaatttagaattgtaatgtctcaactggaggagttccaaaagattccctactattagctccaggaagcagaggctgggccagatgaaatggattgcccacgattctcag

SEQ ID NO: 348 - DNA, 401 bp, Homo sapiens
aaataatcccagtgcctcctctgggtacagtgcagtaggtagggtgagagggtaggtgccaagtgggatctactacacagactggtagagttttacctacctccatacctgtccccactcactcttactctgtcttaggaagccaacgggcagtagaacttctctcctaacactttaccatgaaaaagtttcaatgggcaygccagcaccattttaaggttgttggcagcatcctcaccgtgatgcccactggtatctcaacaaggatggaaaaagtgacagcctttaactatggttcttctttcaacccaagtagttaccatttattgagcacgtactgtgtaccaagaactacagaggtgcatgtctctgacccttgcaacatctccattggataagtt

SEQ ID NO: 349 - DNA, 401 bp, Homo sapiens
tacctacctccatacctgtccccactcactcttactctgtcttaggaagccaacgggcagtagaacttctctcctaacactttaccatgaaaaagtttcaatgggcacgccagcaccattttaaggttgttggcagcatcctcaccgtgatgcccactggtatctcaacaaggatggaaaaagtgacagcctttaactatkgttcttctttcaacccaagtagttaccatttattgagcacgtactgtgtaccaagaactacagaggtgcatgtctctgacccttgcaacatctccattggataagttttataactgcactataagtattattatatttgcactgtatcactttttttttttttgagacagagtcttgctctgttgcccaggtggagtgca

SEQ ID NO: 350 - DNA, 401 bp, Homo sapiens
cccaggttcacgccattctcctgcctcagcctcctgagtagctgggactacaggcgcctgccaccacacctgcctaatttttttgtatttttttagtagagacggggtttcaccgtgttacccaggatggtctcgatctcctgacctcatgacccgcccgcctcagcctcccaaagtgctgggattacaggtgtgagccaycgtgcccagccttctgttttgtttttactttaaattttagcaagaatataagttccacaacagaagtgaccttgcttctcttgttaacaattccattccaagcacctaccatctgcctcacacatagtagttattaataagtgcttgctaaatgaatgagggaatcctcacctcatagggtaggaaactaagtcttttta

SEQ ID NO: 351 - DNA, 401 bp, Homo sapiens
aggttcacgccattctcctgcctcagcctcctgagtagctgggactacaggcgcctgccaccacacctgcctaatttttttgtatttttttagtagagacggggtttcaccgtgttacccaggatggtctcgatctcctgacctcatgacccgcccgcctcagcctcccaaagtgctgggattacaggtgtgagccaccgygcccagccttctgttttgtttttactttaaattttagcaagaatataagttccacaacagaagtgaccttgcttctcttgttaacaattccattccaagcacctaccatctgcctcacacatagtagttattaataagtgcttgctaaatgaatgagggaatcctcacctcatagggtaggaaactaagtctttttagag

SEQ ID NO: 352 - DNA, 401 bp, Homo sapiens
aaaatgggaggccaagtaaaacctggtgctgggaacaggtgatgagcactagacttgacctctctaaatgaaggatgtagaccactagtttcctccaaattacaaccaccagaaatttagttctttcaataatgacttcaaagtttaaaaacctctcattttaagttgtccatgtttggtggcaaaaaaaagaaagaacayatacatggacagaaatgtacaaatgtagagtcattgctttctagatgtcaaatgcagatgactgagtttatcttatgcctgggtttatggcctagtatcatcattggtccagggaagtttgctctcttactttacttgtgtattgacttttttttttttcgaatccactactcccaatctcctccccacacaggcaacag

SEQ ID NO: 353 - DNA, 401 bp, Homo sapiens
tgcctgggtttatggcctagtatcatcattggtccagggaagtttgctctcttactttacttgtgtattgacttttttttttttcgaatccactactcccaatctcctccccacacaggcaacagttctaatgcatttaatatgtatcctcaaatttgtaggtatccctatataatgtagaattgttttgtgtgtatatayatttttaaatttatataaatggcattggactaaggatcgcatttttccccttttaaattcagtggtatgcttgagagatctgtccctgttgctattatctactgggttttacttcatgacgcccatccactacattttactgaatgatttcccttcaaatggaccacaggttaactctatttcctttatgacacaaacag

SEQ ID NO: 354 - DNA, 401 bp, Homo sapiens
tctacttctctgttcttgtaccactaacacatagtttttagttctatagttgcatactctatcttaatatctgatcacagaaatttgccttcttttgcttcttttccaaagtaacttagctattcatggacctatactcttctgtatacagaatagctttcttacattcattcctaaatccactggaattttgttctggawcaccctgaaactgtacattagtatagagagaattggcatgtttatcatcttaaagcatcccatttatatacatggtacagtttgccttttattcacattttcttaaaatattgtccatagatactatgtattccttgttaatttctagatattttataatttttgttgccattatgaaggcatcttgtcttttatttact

SEQ ID NO: 355 - DNA, 401 bp, Homo sapiens
acattagtatagagagaattggcatgtttatcatcttaaagcatcccatttatatacatggtacagtttgccttttattcacattttcttaaaatattgtccatagatactatgtattccttgttaatttctagatattttataatttttgttgccattatgaaggcatcttgtcttttatttactggttactggtggtakggagagatgtaattaatgcttccaagttgatctgtgtcttgcagatttcctgagctccctaatcattttcctgattttgacagatttccattgagatatcatcagcaaatgacaatttaatactcttcctcccaaccattagactgacattgttttatttgtttttatcttagaattgtccagggcttttgatactgtat

SEQ ID NO: 356 - DNA, 401 bp, Homo sapiens
cccatttatatacatggtacagtttgccttttattcacattttcttaaaatattgtccatagatactatgtattccttgttaatttctagatattttataatttttgttgccattatgaaggcatcttgtcttttatttactggttactggtggtagggagagatgtaattaatgcttccaagttgatctgtgtcttgcaratttcctgagctccctaatcattttcctgattttgacagatttccattgagatatcatcagcaaatgacaatttaatactcttcctcccaaccattagactgacattgttttatttgtttttatcttagaattgtccagggcttttgatactgtattaagtagcagcagcatatgtttctaatcttaaaggagatgattt

SEQ ID NO: 357 - DNA, 401 bp, Homo sapiens
tggtacagtttgccttttattcacattttcttaaaatattgtccatagatactatgtattccttgttaatttctagatattttataatttttgttgccattatgaaggcatcttgtcttttatttactggttactggtggtagggagagatgtaattaatgcttccaagttgatctgtgtcttgcagatttcctgagctcyctaatcattttcctgattttgacagatttccattgagatatcatcagcaaatgacaatttaatactcttcctcccaaccattagactgacattgttttatttgtttttatcttagaattgtccagggcttttgatactgtattaagtagcagcagcatatgtttctaatcttaaaggagatgatttcaaagtctaaagta

SEQ ID NO: 358 - DNA, 401 bp, Homo sapiens
tttcctgagctccctaatcattttcctgattttgacagatttccattgagatatcatcagcaaatgacaatttaatactcttcctcccaaccattagactgacattgttttatttgtttttatcttagaattgtccagggcttttgatactgtattaagtagcagcagcatatgtttctaatcttaaaggagatgatttcraagtctaaagtatgttgtctactgtaggttaggggcataaatctttaccacattaaaaaggtccttttctaggcatggtgtgctaagagacttttaacataaatgtatgttcaactttataaaaatgctttttctgtatctattaagatgatcaagatatttttcttttgtggattaatttagcaatacattttctgagg

SEQ ID NO: 359 - DNA, 401 bp, Homo sapiens
atttcaaagtctaaagtatgttgtctactgtaggttaggggcataaatctttaccacattaaaaaggtccttttctaggcatggtgtgctaagagacttttaacataaatgtatgttcaactttataaaaatgctttttctgtatctattaagatgatcaagatatttttcttttgtggattaatttagcaatacattttytgaggttgaaccatctttgcttttcttggctaaaccctccatgatcatctgtttgttactttcactttctaaaatttcattagctaatatttcattttgcattttttacctatatgttcaaaaatcacattggccttttgtactgtccttggcagaatttgcaataaaaaatacaataatctcataaaataatttctaat

SEQ ID NO: 360 - DNA, 401 bp, Homo sapiens
ataaaaatgctttttctgtatctattaagatgatcaagatatttttcttttgtggattaatttagcaatacattttctgaggttgaaccatctttgcttttcttggctaaaccctccatgatcatctgtttgttactttcactttctaaaatttcattagctaatatttcattttgcattttttacctatatgttcaaaartcacattggccttttgtactgtccttggcagaatttgcaataaaaaatacaataatctcataaaataatttctaatgtttcatcattttcctatttcccagagttgtttattttctaacactggtggaatttacttataaaaccactggatctggggcttttgtggaataaagagctttatcaccatttttaatttcatt

SEQ ID NO: 361 - DNA, 401 bp, Homo sapiens
tacctatatgttcaaaaatcacattggccttttgtactgtccttggcagaatttgcaataaaaaatacaataatctcataaaataatttctaatgtttcatcattttcctatttcccagagttgtttattttctaacactggtggaatttacttataaaaccactggatctggggcttttgtggaataaagagctttatcwccatttttaatttcattattgtttattggtttattaaatttttccagtttttcactatttttggcattttgtatttttccagaaatatagttctcatggaaaactttccttgaaagtttccatttcatgtagatttcaaatatattggatagtttttcataatattcttccataatatttttctcagctgtgtctatttt

SEQ ID NO: 362 - DNA, 401 bp, Homo sapiens
aatctcataaaataatttctaatgtttcatcattttcctatttcccagagttgtttattttctaacactggtggaatttacttataaaaccactggatctggggcttttgtggaataaagagctttatcaccatttttaatttcattattgtttattggtttattaaatttttccagtttttcactatttttggcattttrtatttttccagaaatatagttctcatggaaaactttccttgaaagtttccatttcatgtagatttcaaatatattggatagtttttcataatattcttccataatatttttctcagctgtgtctattttatttccctctttttcattctgtaattttgcttattttctctttctctcgatcagtcctgccagaaagtgct

SEQ ID NO: 363 - DNA, 401 bp, Homo sapiens
tttctaatgtttcatcattttcctatttcccagagttgtttattttctaacactggtggaatttacttataaaaccactggatctggggcttttgtggaataaagagctttatcaccatttttaatttcattattgtttattggtttattaaatttttccagtttttcactatttttggcattttgtatttttccagaaayatagttctcatggaaaactttccttgaaagtttccatttcatgtagatttcaaatatattggatagtttttcataatattcttccataatatttttctcagctgtgtctattttatttccctctttttcattctgtaattttgcttattttctctttctctcgatcagtcctgccagaaagtgcttctcttattgatgct

SEQ ID NO: 364 - DNA, 401 bp, Homo sapiens
tatttccctctttttcattctgtaattttgcttattttctctttctctcgatcagtcctgccagaaagtgcttctcttattgatgctttcaaagtactagattttggttttattaatcttatttttagtgtaccttaaattttatcataaatttattatatctctataataaattttgttctttttggggaacaaaatttkgggggaatttgttccttttccaacttcttcatttgaacacaaataatttgttgttggtctttgttgctttcaaataaatgtgtctaaagctatatctttccactggttactattttagctgtcttacaaattttgatagtttttaaaacatttctgggccgggcacagtggctcatacctgtaatcccagcactgtggga

SEQ ID NO: 365 - DNA, 401 bp, Homo sapiens
taatttgttgttggtctttgttgctttcaaataaatgtgtctaaagctatatctttccactggttactattttagctgtcttacaaattttgatagtttttaaaacatttctgggccgggcacagtggctcatacctgtaatcccagcactgtgggagaccaaggcaggaggatcatgaggtcaagtgtttgagaccagcytgaccaacatggtgaaaccctgtctctactaaaaatacaaaaattagccaggcgtagtggcgtgtgcctgtaatcccagctactcaggaggttgaggcagaagaatcacttgaaaccaggagatggaggttgcagtgagccaagatcacgccactgcactccagcctgggtgacagagcgagactccatctcaaaaaaaa

SEQ ID NO: 366 - DNA, 401 bp, Homo sapiens
tttagttttaaatatttcatcatttctgttaatcattgtttaaccaagttatttgggtttcaggtacatgggttactttttagctttctgttgcaactgattccttattttactgcatctttatcattgaacatatgatatattaaacaggattctttttggaatatagaacttttcttggtagcctagtaaattgtaaaygttttatgaataatccattctcaaaataaaatgtacctttactgtttgttgggcatagaattccatacatatctactggtttctccacttattgtattattcggatcttttatatctttgcttgtctttggtctgcttaacctatcagtttgagaaagctgttttaaaatttctgtccacaattgttgacttacccgttt

SEQ ID NO: 367 - DNA, 401 bp, Homo sapiens
atctttatcattgaacatatgatatattaaacaggattctttttggaatatagaacttttcttggtagcctagtaaattgtaaatgttttatgaataatccattctcaaaataaaatgtacctttactgtttgttgggcatagaattccatacatatctactggtttctccacttattgtattattcggatcttttatatytttgcttgtctttggtctgcttaacctatcagtttgagaaagctgttttaaaatttctgtccacaattgttgacttacccgtttcttcccatagttgactgacattttacatatttttaagacaatattatcaggtacatttatgttcattatgattatatattattctattgctacacttataaatatgtcacatcctt

SEQ ID NO: 368 - DNA, 401 bp, Homo sapiens
aagtggacccatgtagttcgaacccatgttgttcaagggtcaactgtaattttttttatccttttgttattaccctaaccagattgccgtccatcactactctcttactgacaaaatattgctggatcttaatattgtatccaatctgagtatttctgtcttttgatgggtcaatttatatgtattgcaattatggttatrttaggatttacttctgccatcttattttacttttttctctatttattacactttcttttgtgtatttttttctttttgtttcttgctttccattgggtaaactttccacctgtgaggaacttatacattctagttttgttttctggtagttgctcctaacttgttaagatccgtagttacattctccccgtctatttctt

SEQ ID NO: 369 - DNA, 401 bp, Homo sapiens
tttgtgtatttttttctttttgtttcttgctttccattgggtaaactttccacctgtgaggaacttatacattctagttttgttttctggtagttgctcctaacttgttaagatccgtagttacattctccccgtctatttcttcatcttaacaatacttacatctttcactgaacaaaaaaagtgcctgtccccacctgsctctatcctcaccttcttctcctagcccatcaacctaggttcattatgttgcaaattttattttgggttgttgtgcacatcaacaatggtagttctgaggacaaacatcaatcaatctttcttctggaggcagatgacttcaaagacatttctttggggtcttccttcttcacccaagtaaatttgaaagatgtatgcta

SEQ ID NO: 370 - DNA, 401 bp, Homo sapiens
ttaacaatacttacatctttcactgaacaaaaaaagtgcctgtccccacctgcctctatcctcaccttcttctcctagcccatcaacctaggttcattatgttgcaaattttattttgggttgttgtgcacatcaacaatggtagttctgaggacaaacatcaatcaatctttcttctggaggcagatgacttcaaagacrtttctttggggtcttccttcttcacccaagtaaatttgaaagatgtatgctagagagtgggctgctttctcctggtagagtatagtatagctgttgactcacaggcacttaacaaaaataaaccttaccacatgaataaacatcaaatcatcacaaacaacactttagccctggaaacctggtgggtgagaagggaatgt

SEQ ID NO: 371 - DNA, 401 bp, Homo sapiens
caagcaatctcacttcattgctggatgatagcccagggtgctgccccacgtcccaaaccacactcttctaaacagcagggtacctatgtttatgaagggttgtaagtgaaaggtacacattaagcaaatatgcataagccaagacatgcccatagtgataatttcacacacatatactgcagtacaaacatacctcagtgktagttatatgtgtttctacacacaaacacacacttatatacacacatactgtctctaaacgatactttagtttgacaatacagtacttaaactcccctagactcatttttttttaacataagttccatattatcataagcactgggattcaatagcaggcaaaagctattaagcccaaggagaaccaaaacctaaaacac

SEQ ID NO: 372 - DNA, 401 bp, Homo sapiens
ctttgttgaaacagctccttcagctcttcgtttgcagctttctctctcaccagggctcagacctcatggtccatggaggcaggctgggaaggagggggtgggtcactaccatctccaaccttcctcccaaagacacacgtagagaaaccttcacaccaaactccgattccaccactttctccaatggggataaatattaaracattatatttagccatgggatattaagtcatgacacagttgtaaacccaaacaaaccccaaatcttggtttagacttggttgaaacatctgctatgctgatgactccttttgtaacttttctctccctgaaaaaattctggatgttttgagatgcccattttattccacaaatatttttgaggtcttgtcatgaaggca

SEQ ID NO: 373 - DNA, 401 bp, Homo sapiens
tagttctcaaccagaggctagagatgggagaatcttgccccccaggagatatttggaaacgtcttggggcacttttgtcccatctgaaggcagggggcactaatggtatgtagcacatagaggtcagtgatgcagtgaacagcagcccccacttcccaggtagacatttctcctttgtactggccaaatgccctccttcaytgagaacagaaaggagaaaatgttttgatggtctaaagcggaagtccccaacctttttggccccagtgactggtttcatggaagactatttttccacagatcaggagggtggggagatggtttcaggatgaaactgttccaccttagatcaacaggtattaaattctcataaggagtgtgcaacctagatccctcccatg

SEQ ID NO: 374 - DNA, 401 bp, Homo sapiens
ccgcccagttcctcacagaccacggaccagtactggtctgtgactcaggggtgggggacccctggtctaaagctcaaggaggtggaacataacattccactccagtgtgcaccttacccggtggcttccttctcaggagcacagtatgtaagcagggaaaggagtaacctgctttgcagcagagaggcctggcaaacactyctcagcaggcagttaatgtcaacatcaacagccatgagtcatgttgatagaatgtaccctagacacggtatgatgagaatagcaccttacctcccccaaacccataaccctaccctgagcatgaaaaaaaaaatcagaaaaaaaaaattccaattgagggacattttatgacatacttgaccagtatttcagatcaaaat

SEQ ID NO: 375 - DNA, 401 bp, Homo sapiens
aaatgaggaaagtcattgaaaacaagggaagtctgagaaactgtcactatcaaggggaaccttaggagacatgacagctaaacatgatatgagatcctgggtgggatcctcgataggatcctgaaaccaaaaaacgagattagatgaaagctacggaaatcagaatagcacatgcacttaataataacaatatactaataytaattcatttgttatgacaaatgtaccacactaataaaacatgtcggctaggcatggtggctcatgcctgtaattccagcactttgggaggctgaggcaggcagatcacttgagttcaggagtttgagaccagcccggccaacatggtgaaaccccatctctactaaaaatacaaaaactagctgggcatggtgacacat

SEQ ID NO: 376 - DNA, 401 bp, Homo sapiens
attaaagttggccaggcacggtggctcacgtctgtaatcccagcattttgagaggccaaggagggcggatcacgaggtcaggattttgagaccagcctgaccaacatggtgaaaccctgtctctactaaaaaaaatacaaaagttagccaggtgtggtggcacatcctgtaatcccagctactcaggaggctgaggcaggygtatcacttgaacctgggaggtggaggttgcagtgagccaagatcatgccactgcactccaggttgggcaacagaacaagactctgtctcaacaaaaagaaaaaaagaaattagaattgttgggagaattaacctaatggacacaaagtccttaacatgctgcctggcccatagtgatgcccaataaatgttagtttgct

SEQ ID NO: 377 - DNA, 401 bp, Homo sapiens
ggtgtatcacttgaacctgggaggtggaggttgcagtgagccaagatcatgccactgcactccaggttgggcaacagaacaagactctgtctcaacaaaaagaaaaaaagaaattagaattgttgggagaattaacctaatggacacaaagtccttaacatgctgcctggcccatagtgatgcccaataaatgttagtttsctcttttaatttcattacgccatttttagcctgttctacttgccaaccttgcaagaggtaagaggtctcatgtttcaaaagcaaatcatgcttattcacatttccatactcatacctccctctacgtggagcacccttctttcaagatccttacagatgaggtcatctccttccacaaacctgtcctttccaacacccct

SEQ ID NO: 378 - DNA, 401 bp, Homo sapiens
tcacttcgaaaggaaattccgtagccattaagcagccactccccattcccacctgcccccagttctggcaaccacaaatctttctgtctctatggatgtaccaatttttgatatttcatgtaaattgaatcattcaatatgtaacccttagtgtctggcttctttcacctaacatagtattttcaaggttcatccatgtcrtagcatgtatcagcactttgttcctttttatggctgaatactattccattgtatggctatgccacatttttattcatttgttgatagacatttgagatgtccacatcttttggctattgtaaataggtctgctgtaaacattcatgtatttctttaatggctgttttcagttctttagggtatatacttaggagtagaat

SEQ ID NO: 379 - DNA, 401 bp, Homo sapiens
cccagttctggcaaccacaaatctttctgtctctatggatgtaccaatttttgatatttcatgtaaattgaatcattcaatatgtaacccttagtgtctggcttctttcacctaacatagtattttcaaggttcatccatgtcgtagcatgtatcagcactttgttcctttttatggctgaatactattccattgtatggstatgccacatttttattcatttgttgatagacatttgagatgtccacatcttttggctattgtaaataggtctgctgtaaacattcatgtatttctttaatggctgttttcagttctttagggtatatacttaggagtagaattgctggatcataggctaattctatgattaaatgtttgaggaaactccaaactgtctc

SEQ ID NO: 380 - DNA, 401 bp, Homo sapiens
ctggaacatgttggttattgagctttataccctctgtgccctcgaggctgtgagctcaatgacagtggaggacgccgctccttctcctttgtgtcttcagtgcaaagggcaactgacatcgtaggggaaaaaatacccaaaacctcttctcttaaaaatgagccgaatgaagtaaagaatagcccaagagattctgccaawggtagaagctgtgagaatcagggtggagaggacaccaaccaggtggaatgggcacaccaatgaggtgggacatcaactgggacttgagggatgagcagattttgatctagatccccaattcctcctttcatctccattaattcatttgttcatcaatgcaataaacagatcctgagtgattacgtcgtgctgggaggcag

SEQ ID NO: 381 - DNA, 401 bp, Homo sapiens
aaatgttgaaattcttatttcctaattcctaattctataattacagtgctcagagatgatcttctgctcactgatatcatgaagaaaagttatcatacatatctgcacacacctgaaactttaaggaggcctttcacaaagatgatctcatttattttcctcaaaaacctgggaagtacacactaccactctcagttgacrgaataaactaaagaataaagaggtgagatgccttgcccaagtccacaccattcacaagtgacaaagccaagacttcattttagcccttttacttttaaatcttctgatatttctcatctagaagagcattacagaaattgtagcccaggaacacattatgttgtacctgtgatagtattttttttctttttttctttttt

SEQ ID NO: 382 - DNA, 401 bp, Homo sapiens
gttaaaaattcaacaaatattgtaatatcaatatctgagataaaccttatcagaactctgatattcccaagaaattaataaatgactggctgcgtttcataatgcaaaaataccaacttgtcttactgtgacacaagggctaaaatgaccatgatggtgatgatgaaggagtggtgatgatgaaggagtggtgatgatgawgatgatgatatgatgatgatgatgatgatatgatgatggtggtgatgaggatgatgatgatagtggtggtgggagtgccagtggtagtaatgacaaggaggaggaggatgatgatgatgatagtgatggtgtcagtggtagtgattatgatgatgagatactggtgaaggtgcccatggttgtgatgatggtggtggtag

SEQ ID NO: 383 - DNA, 401 bp, Homo sapiens
aaaaattcaacaaatattgtaatatcaatatctgagataaaccttatcagaactctgatattcccaagaaattaataaatgactggctgcgtttcataatgcaaaaataccaacttgtcttactgtgacacaagggctaaaatgaccatgatggtgatgatgaaggagtggtgatgatgaaggagtggtgatgatgatgawgatgatatgatgatgatgatgatgatatgatgatggtggtgatgaggatgatgatgatagtggtggtgggagtgccagtggtagtaatgacaaggaggaggaggatgatgatgatgatagtgatggtgtcagtggtagtgattatgatgatgagatactggtgaaggtgcccatggttgtgatgatggtggtggtagttc

SEQ ID NO: 384 - DNA, 401 bp, Homo sapiens
ttcaacaaatattgtaatatcaatatctgagataaaccttatcagaactctgatattcccaagaaattaataaatgactggctgcgtttcataatgcaaaaataccaacttgtcttactgtgacacaagggctaaaatgaccatgatggtgatgatgaaggagtggtgatgatgaaggagtggtgatgatgatgatgatgrtatgatgatgatgatgatgatatgatgatggtggtgatgaggatgatgatgatagtggtggtgggagtgccagtggtagtaatgacaaggaggaggaggatgatgatgatgatagtgatggtgtcagtggtagtgattatgatgatgagatactggtgaaggtgcccatggttgtgatgatggtggtggtagttcagacc

SEQ ID NO: 385 - DNA, 401 bp, Homo sapiens
tttgggaaaatatacttaatttttactctaaaagttatatatgttaatatgttatgggttgattattgctattttttataatactttaagttctggggtacgtgtgcacaacatgcagttttgttacataggtatacatgtgccatggtggtttgctgcacccatcaacctgtcacctacattagctgtttctcttaatgytatccctcccctaacccccacccaccgacaggccccagtgtgtgatgttcccctccctgtgtccatgtgttctcattattgctattttatgtgaaataatacttttaagttttcctcagttttaattcccaatataataaatattaacagatataacccgcattccaaaagcttttaggaatctccaataatttttaaga

SEQ ID NO: 386 - DNA, 401 bp, Homo sapiens
cgctgcccccagcggggcgcgcctcaccctggactgtggggtcctctccagagccgcccatctggtcctgagacttggcccaaggctttgggtccacagcctggctctaaggaaagcctgtgccaggccttggggaacccattgccgagcagtcacagaaagctcccggggtccccggggcccatctcagtgaagcctgasgcgggggatgggggtggggagcagggcccttcccgctggagcctggagcctgtgcctctgggtcccctgtggggtgggggcctcatagccatgctcccagggtgggatgcagcgccctccccgctggagcctggagcctgtgcctctgaggcccctgtggggtgggggcctcacagccatgcatccaggtgcctggttct

SEQ ID NO: 387 - DNA, 401 bp, Homo sapiens
tgtctgtctctgtctctttctctgtccgtttctgtctctctgactctatctctgtctctctctgtctctgtctatccctgtctctctttctctgtctttctctctctgtctctccctatctctgtctctccttgtctctctctgtttctgtctttctctctctctgtctctgtatctctctgtctcccccatctctctctstctgtctctgtctttctctgtctctatctctctgtctttgtctatccctatctctctctgtctctgtctctctctgtctttctctctcttcctgtctctctgtctttctctctcttcctgtctctctgtctctgtctctctgtctctgtctccccatctctctctttttctgtctctgtctctccatctctgtctctccc

SEQ ID NO: 388 - DNA, 401 bp, Homo sapiens
agcgcactctctctctgtgcatcttagtgagggccccagcatgcatgccttgtcagtgacaccagatttcctaattgatgcgggtcatcgagaccaaatcgaggggaagcaggcagccaggtcctcctggttccctcttggaagcctcctgagtgtctgggattgtccttcgaggttaaaaaccatttacgcgtgcctccragtctcctgtgtcttaacagattttgccctccttggagagcctgtgccaggtgggcaggctcaagggacactttcctgaggaagggcatttccaaactcagccgccccactcacctgagccgtctcaagggcgtgctgcacctgggcattctcacctgggaaatgggtgtgatggcggagccccccgagggtggtgctgg

SEQ ID NO: 389 - DNA, 401 bp, Homo sapiens
gaggtggcgtgcgggagccggaggagccaccccttccaggaccatgtggcgccagcacagctgtttccagctgtctcttctttgctgctcaacgcctggccccggggtcagctttccccttagtccatctgcagctaattcttagcaaaaatccatgcagcatttacttggtgccacaaacaccacctgctggagagagaraaaaggggaagcttagtccctgcgtgagaactgcagctccctcctccccctctctcctggtttcctactttttcaaggacagacaggaggaactgtttctgtgcccagtgtctctttccctctgggtgctttccatttggccacgtccccaccccattctctcggttacctgcttccacctgagtgagccaacatctgca

SEQ ID NO: 390 - DNA, 401 bp, Homo sapiens
acagagaaagatgctgtctcaaaacaaacacccaaaaaggagttgtgtagacattcttaccatgtggaatcaacgcttcttcttccttcaaattcggaaaaaatgcatggactgactgtctcttcctgcgcattcaacgtaaagactgcccagtgagacccgtgcccacgcaggccggggaaagtgccctccgtgctggayttgggttggccaacagagccaccacccccaaccacaacactctggttgattttttatctacatttatgcccacgtggcagtctacatacggctttaatgcagccgctgtaccacagagtcgacgttcctgtttcgtgaagatcccagctgtggccacgatctcgtattcacagaaaaccctctcatctcctctcccctaa

SEQ ID NO: 391 - DNA, 401 bp, Homo sapiens
ggggtcacgcttagaccccaagtcctagtccagggccggctgcctccttttgggccccctgctcccctccagggccctgccctcttgatgagaggtctcagcaaccgagccaaaatcagaggcagggtttggcaacccaacagtgccccaagggtgtctccaccacccaagtggtgcccccaacattcaggtcccgtctgycccttgaagaccacaggtggctccccctggcccacgccacagtcccccccagcccccgaccctgggcaggtgctgaccaccccctcctcacaggggccttcctcatcccctacgtcatcgcgctggtcttcgaggggatccccattttccacgtcgagctcgccatcggccagcggctgcggaagggcagcgtcggcgtg

SEQ ID NO: 392 - DNA, 401 bp, Homo sapiens
gcggccaggccaggccggcgggtggggtggcagggagcccttgggtgtgtgtgagaagcagcggtgactcggggagaattagagatggagaacatgtgtgccaggacatcccggaaggacctggaagctggtgttgccattcacatgtgaggtgtgagggaggcctggctttcagctgcgcccttcagcatgtgttatttwatgttgttattttgttttattctcactgcttctgggcagagggagctgggaaggagccccggggccacctgacatggtccctgtccacagggctgggctgtgtcacgctgtccttcctgatcagcctgtactacaacaccatcgtggcgtgggtgctgtggtacctcctcaactccttccagcacccgctgccctggagc

SEQ ID NO: 393 - DNA, 401 bp, Homo sapiens
ctccacactcagctaattttttttttgagacggagtcttgctctgtcgcccaggctggagtgcagtggtgctatcttggctcactgcaagctctgcctcccaggttcacgccattctcctgcctcagcctcccaaatagctaggactacaggcgcctgccaccaggcctggctaattttttgtatttttagtagagacggrgtttcaccatgttagccaggatggtctcgatctcctgacctcgtgatccgcctgcctcggcctcccaaagtgctgggattacaggcgtgagccaccgcgcccggccagcacccagctaattttattttttgtagagacggggtctcaatatgttgcccaggatgatcttgaacccttgggttcaagcgatcctcctgctt

SEQ ID NO: 394 - DNA, 401 bp, Homo sapiens
ctgctccaggagcctgcacggcttctagagcattctgtactatcccagatcgcggcactccctgcccagcatcccgaggcagtttccaggggctggtgtgggagtgggcacctgggtatgggcatgtctgcccggagtcctagcaaaagacagaggacaccttcccaggccccagaagcagaaaagccaggctccgtgcayggcatccctgccctcggggcttcaaatcacccagaaccgcccggttagagagggcacagtaaacatgcccatgcaggagctgctcaaacccatctccagccccaaaaaacctgccagctacgttctcatgtgcgcctgacagccaccggctcattcggccacatcccggacaggggcctggcatttgccgtgtacagagc

SEQ ID NO: 395 - DNA, 401 bp, Homo sapiens
tgtgcaggggagctggggaagccctgatcttctgactaaagccctccacagtcagcctgggcgggacacggagaagctaataccatggcctgggctcagccaggctggcatctcccagaccagcgctcccgtccctcactgatgcaaatccagcttgaatctgtcacaatctcacactctcatcagccgaacattgcagamaaaatctcacagccattcaaatgcagccacctagaagttagctcatggtggaactctcctatccccaggtgctccctgcacaggtgctccctgcagtgagtcgccaagtgggtgggattttatcctctcatgacctaaatccaatagattgaagattcatacagaacagcatgaattaaaagagaatcgggggctgggca

SEQ ID NO: 396 - DNA, 401 bp, Homo sapiens
ccactagggcaaaggggcggtttggaaaatttgttccaacaaaagttaagttgtagcttacactggttctctgcagagaagccaacatagaaaacacaattttaaaagagggaagagaagaaatggaagcagaagattatgctggagtaattaacaccatgtgcatggcgaggaaacgcctcccggcattcaatgaagatygctgatacccagaagacaccccagtattatgggtgcagttagtgtgtctttgaaaagctgatgatgtcttagtcatcacagtgtaaaacatcaagagtgttctaacaacaataaaaaaattctatcattggcttaaaacaccacaacacttgagtggggtgagcttcctacctcagacccagatgtttctaaacagagta

SEQ ID NO: 397 - DNA, 401 bp, Homo sapiens
ttctatcattggcttaaaacaccacaacacttgagtggggtgagcttcctacctcagacccagatgtttctaaacagagtaaattctgagctgggcatggtggctcacacctgtaatcccagcactttaggaggcagaggcaggtggatcatctgaggtcagaagttcgagaccagcctggccaacatggtgaaccccacmcccacccccgtctctactaaaaatacaaaaaattagctgggcatggtggcgggtgcctgtaatcccagccaaatgggaggcggaagcaggagaatcacttgaacctgggaggcagaggctgcagtaagccaagatcgcgccattgcactccctccagcctgagcaacaagagtgaaactgcctctcaaaacaaaacaaac

SEQ ID NO: 398 - DNA, 401 bp, Homo sapiens
acagaaaatatcaaaggagggtaactcctgagtaaaggggaaattgaactccatgaactccttaccagtgggtgtgacttgagcccagggaagttttaaaatggttaaataggccaggtgcagtggctcatgcctgtaatcccagcactttgggaggctgaggtgggcggatcacctgaggtcaggattttgagatcagcytgaccaacatggtgaaagcccatctctactaaaaatacaaaattagccgggtgtggtggtgcatgcctgaaatcccagctacttgggaggctgaggcacgagaattgcttgaacccaggagacggaggttgcagtgagccaagatcatgccgctgcactccagcctgggcaacaagagcataactctgtctcagaaagta

SEQ ID NO: 399 - DNA, 401 bp, Homo sapiens
gttccagagggaggggggcagaaaagtgctgggcagagaagggcgggtccctggcgggggctccatccccaggcctgtgcccgtggacctaggtgaggacaggcactcctactttcacacccaaatgttgcatttcccaagagcgccctggcccaccacgcccccgtcctatgcctataaaaacccccagaccctagcggrcagagacacacgcagctggacgtggaaaggagcacagaaacagaagaacacaccggcaggccccggcagatgacaggagacggggagcgcctggaaatggaggtcagcgtgcagccggacgccaggaacctgcatcgggggccacgagccaggttcaggctcagaggagccagtctgggaggcgcagaatctcctccctt

SEQ ID NO: 400 - DNA, 401 bp, Homo sapiens
tcatgttcgtttccacgggttcagagctccgtgacttcctgggttagtgtgggtgggcccaggaagccggtatggggtcctgagtcacacgcagcgtggccgccttcgcgttgcccaatgtggcagctcccagggaggacggtcagtggtcctgctgtcagcctcagcctcccaggccggggactctctccccggtggccytgccgtgctcatagccctgccgcccagtcctctcctcgtgtactttcccttgcaccctaagccaccttatgaaaaccaataaaaagtacttagggcgaaaaatccctctgaacatggttattaagtccttggaccttggcaaacatagctcagttcctgttttacgcctttggatttctgcactcctgaatgttatttga

SEQ ID NO: 401 - DNA, 401 bp, Homo sapiens
tcccaaagtgctgggattacaggcatgagccactgcgcctggcttttctacttttttgatgtagctgcttatagctataaacttccttcttagcactgcttttgctgtataccgtgggttttgagaggttatgtttccgttatcatttgcttaaataaatttttcaatttcctttttaatttcttcattgactcactggtyattcagaagtatattgtttaattttcatgtgtttgtatagttttcaaaattcctcttgttgttgatttctggttttgttccactgtggccagagaagatgctttatattatttcaatttttttttttttttttgagacagggtctcattctgtcaccaaggctggtgtgtggtgttgcaatcatggctcaccgtagcctt

SEQ ID NO: 402 - DNA, 401 bp, Homo sapiens
taagccatcatgcctggctcaatttttttgaatttttaatggcttgttctgtggcctaatatatggtctatccttgagaatgatccatatgctgaggaaaaaatgcatattctgcaactactggatgaaatgtcttgtaaacatcaattaggtacatttgttctatattatagattaagtccaattttttggttgattttstgtctggatgatctgtccaatgctaaaagcggggtgaagtctccagctattattgtatggggatctctctctctccttagttctaataatatttgctgtatatatctgggtgtcccagtgttgggcgcatataaattcacaattgttatatctttttgctttattattatataattactttgtctctttttgtacttttt

SEQ ID NO: 403 - DNA, 401 bp, Homo sapiens
cttgggaggctgaggtgagaggatcacctgtgcctgggaaggtcatagctgcggtaagccaaggtcgcacccctgtactccagccttggtgacagtgagaccctgtctctgaggccaggctctactgagtactccagtcaatacctggtgaattagggctgggggcggtggctcatgcctgtaatcccagcactttgggargctgagatgggtggatcacttgaggccaagagtttgagaccagcctggtcaacatggtgaaaccctgtctctactaaaaatacaaaaattagccaggcatggtagcatgcacctgtactcccagctactcaggaggttgaggcaggagaattgcttgaacccgggaggaagacattgcagtgagtcaagatcgcgccact

SEQ ID NO: 404 - DNA, 401 bp, Homo sapiens
gtcaacatggtgaaaccctgtctctactaaaaatacaaaaattagccaggcatggtagcatgcacctgtactcccagctactcaggaggttgaggcaggagaattgcttgaacccgggaggaagacattgcagtgagtcaagatcgcgccactgcactccagccttagcaatagagtaagaccttgtctcaaaaacaaacraacaaaaacaaaaacaaaacaaaacaaataacaacaaaaaaacctggtgaattagaagcttttctgctctggctaattagaaccggcactattgctgtgggaacaccattccttccaatcctattgggttgttcttatttggaccttttcctgtaatccagtcctttttctgacacacatgccgaccagccatcagccac

SEQ ID NO: 405 - DNA, 401 bp, Homo sapiens
tcctaacccccagaacttcagaatgtgcccttatttggaaatagaatatttgcacgtgtgattagttaagatggggtcattagggtggccctaagccagtgactggtgtccttataagaaggagaaatctggacacagagacagacgggcatcaagggaagatgatgtgaggacacagggagaagacggccatgtacaagycaaggagaggctgacacagagcatccctcgcagcctcagagggagccagtcctgcccacacctgatctgggaccgtggcctccagaactgagttggccaatgtctgctattgaagctgccagccacagtgctttgttgtggccgcccccacaagctcacacaggggtggagaagcagacgttgccacaggtgtcacgtaa

SEQ ID NO: 406 - DNA, 401 bp, Homo sapiens
atgccacagtgcacagccagagtggcacagaccccttagaacacgggtgacatgtactggagctcagcacacatgcaccacaacagccatgtcccccctgggtctgtgcccaaaggcaggcacagccacgtgcatgatttgatccgtaacggccccggttcgtgccatggaatgccacatggaagaaagtcacaatgagcrccgcttgcgacaacacagatggacgccagacagagcccagcccgagaacctgggtaccagagctcccagcatgactgttcatgtgtgggacttggacccggtgacccatgggctgggttcacccgcctggtttccactgagagggccccgtggggccggggtctgtctgtagcctgactcgggtgcttggtgctggagtc

SEQ ID NO: 407 - DNA, 401 bp, Homo sapiens
gtaatctcttactgtgcttaacttataaattgaactttatcaaaaccattacataatacatatattcattcattacgttatttgatacacatattcagacatccaatgagggtctttgaacacaccccctgaggatgagggactactgtataacaccagatgaggataaggggcggactactgtatatacactggatgagracaaggggggactactgtatacacacgggatgaggataaggggggaatactgtagacacaccggataaggggggactactgtatacacaccggatgaggataaggggggactactgtatacacaccggatgaggataaggggggactactgtagacacaccggataaggggggactactgtatacacaccggatgaggat

SEQ ID NO: 408 - DNA, 401 bp, Homo sapiens
attcagacatccaatgagggtctttgaacacaccccctgaggatgagggactactgtataacaccagatgaggataaggggcggactactgtatatacactggatgagaacaaggggggactactgtatacacacgggatgaggataaggggggaatactgtagacacaccggataaggggggactactgtatacacaccrgatgaggataaggggggactactgtatacacaccggatgaggataaggggggactactgtagacacaccggataaggggggactactgtatacacaccggatgaggataaggggggactactgtatacacaccggatgaggataaggggggactactgtagacacaccggataaggggggactactgtatacacaccaga

SEQ ID NO: 409 - DNA, 401 bp, Homo sapiens
cctgaggatgagggactactgtataacaccagatgaggataaggggcggactactgtatatacactggatgagaacaaggggggactactgtatacacacgggatgaggataaggggggaatactgtagacacaccggataaggggggactactgtatacacaccggatgaggataaggggggactactgtatacacaccrgatgaggataaggggggactactgtagacacaccggataaggggggactactgtatacacaccggatgaggataaggggggactactgtatacacaccggatgaggataaggggggactactgtagacacaccggataaggggggactactgtatacacaccagataaggggggactactgtatacacaccagatgagga

SEQ ID NO: 410 - DNA, 401 bp, Homo sapiens
aggggggactactgtatacacaccagatgaggataaggggggactactgtatacacacacgggatgaggataaggggggactactgtatacacattggatgaggataaggggggactattgtatacacaccagatgaggataaggggggactactgtatacacaccagatgaggataaggggggactactgtatacacacsggatgaggataaggggagactactgtatacacacgggatgaggatgagggactattgtatacacaccagatgaggataagggggactactgtatacacaccggctgaggataaggggggactactgtagacacaccgcaagaggataaggggggactactgtatacacaccggatgaggataaggggggactactgtata

SEQ ID NO: 411 - DNA, 401 bp, Homo sapiens
tgccctgcctcaagccctgacccgccaccctccacgtgctggaacatccagcgagggctgtgctctaatccacctgctctgctatggcgggcactgctggtgtcagggtgtggtggaatcagagccacaaccagcaggtgccaggttgccctgagagcggctgtcagggcgccccacgtagccaagtggcatcagtgcacrtggatggtggcctccaggcacccctcgagctgccaagcgtgtccgaagggtgtcactgcctcctccatctggcagtgctgggcccagccacagaaggtgccgacttcctgcacctgctgcatcccagctgcctgcttcctctctcaagacagcacctctcgaatctggccccaagtgagacacagcaacagcgacacatg

SEQ ID NO: 412 - DNA, 401 bp, Homo sapiens
cagaagtgaaggaggcagtatgttagggactgcaaggggtctgagggaacagcagtacagggggactgttctgagagccgcagtcaggaggaggcagcagggcctgggcagagatcaaagcagccaggtgctcgcttctccgtttcccagctgcacccccccgctagcaagccctctgctctccccaggactgtttcctcwtcaacaaaaaaggggaaagggcccgcctcagaggtcagaggtgccgagagccttaagtggaagtggccggctcctagtgggtgatccggagtgacagtgatggcttgcagggaggcagggcagggcagcgatcctgcagggccccagctgagcgttcacctctgaaccccaaataacctcatccgtgaaacaacgccaca

SEQ ID NO: 413 - DNA, 401 bp, Homo sapiens
ttctgagagccgcagtcaggaggaggcagcagggcctgggcagagatcaaagcagccaggtgctcgcttctccgtttcccagctgcacccccccgctagcaagccctctgctctccccaggactgtttcctcttcaacaaaaaaggggaaagggcccgcctcagaggtcagaggtgccgagagccttaagtggaagtggcyggctcctagtgggtgatccggagtgacagtgatggcttgcagggaggcagggcagggcagcgatcctgcagggccccagctgagcgttcacctctgaaccccaaataacctcatccgtgaaacaacgccacagctcccagcaccacaattacggaaagattcacaaagtgcttagcacagtgcctcatgtgcagtaagag

SEQ ID NO: 414 - DNA, 401 bp, Homo sapiens
tggtacagaaacattaactgtcctgaaacagaacaatcatttccactacatacatattatattctgggtctctagaacctattcaaatacctttataaagctgcttcaataaactaattttagctcagaagtactaaaaatgaaacccacctgcccagggctttacgagtcgtgtgtgtgtgtgtgtgtgtgtgtgtgtgygcacgcgcacgcgtgcgcgtcctgagaactcggcacaggtgtgggcgcctacagccgaaagcaaaacggcccgcacttcctgcctccatctcaggaggctgagaggtcaataacccacctgaggccacaaggctgaagcagccagatggaaagcagcttctgggcctggccagggtaccagctgaccacacacactgaat

SEQ ID NO: 415 - DNA, 401 bp, Homo sapiens
aatgtggacacaccctgctgtgagggctgtgggcggtcctgagactggagccagcccccaggctcagctcagctcggggacctcgtgcccgggctttcctggggaggaaggtgttggatctcctgcctctctgtattcattcacactttgggtttctctctcttccgcgcattctaacatctgcaggattttttttttttyttttttttttttgagacagagtctcgctctgttgcccaggctggagtggagtggtgcgatctcagctcgctgcaagctccgcctcctgggttcacgccattctcgtgcctcagcctccagagtaactgggaccacaggcgcccgccacgacgcccagctaattttttgtatttttagtagagacggggtttcaccgtgtt

SEQ ID NO: 416 - DNA, 401 bp, Homo sapiens
ctggcttgtgggccggtgtttgagaatcttgccagttgaatgtgggcatctgttgagggtttcccaaaggctaaccgttcgtgccagagggaggccgtggctcattcctagggccctgggtgagactggggctcatgcacacacaggtatcgatggcatgggctgtaacgagctgagtgcctgctgccctttcctagtggsgggggtgggctcctggagaaagaggggaggctgctactctccagggccacagggagcccagaacgccacctcctggtgtcagcagcagcaaatccatataagtctgcagcaacttagcttttgcctcctcagaggaaataattcatccagggggcacaaggcagggtcagagactgaggtaagtttcagagcaagagtga

SEQ ID NO: 417 - DNA, 401 bp, Homo sapiens
ttattaaaaaagtgttagagcaggaacgaaaggaaataaagtacacttggaggagggccaggtgggcatcttgagagagcaagtacacggttttgacctttgacttggggtttatatcttggcatgcttctgggggctgcgtcccttctcccctgattcttcccttggggtgggctgtccgcatgcgcagtggcctgccgrcacttgggagggccgcgtgcacagtgtgcttactggagttgtgcggtgccctcttgaggcagtcttcccttaccagttcctaggggaaggtcacacgctggttaaactttgccactttgcctcgtagtgtgcatgcttgacctcactcaccaactcctgagattttttttaaattttttaatttaagttctagggtacat

SEQ ID NO: 418 - DNA, 401 bp, Homo sapiens
cccagctaattttgtagttttagtaaagtcaggttttcaccatgttggccagggtggtctcgaactcctgacctcaggtgatccacctgccttggcctcccaaagtgctgggattataggtgtgagccaccacgcccagcctattttcttttttttaagtaaaattttgagacagggttttgctctatcacccaggctggrctggagtgcagtggtgccatcttggctcactgcagcgtcaccctcccaggctcaagcaatcctcccacctcagcctcccaagtagctgggaccacaggggttcaccaccacacccaggcaatatttgtatttcttatagagatggagttccaccctgtttcccaggctggtcttgaactcctggactcaagcaatcctcc

SEQ ID NO: 419 - DNA, 401 bp, Homo sapiens
atcttttttggattcaaatatgtcttcttattggcttttcagctagatctttttgctttacttgttcagtgtttttttctacaattgtcaatttgcagcttaaacttaagctagtctacttccagatattataacccttcatgtggattttaatgactttaaacagtaacgttccatttctgttctattcctttctttatwgtcatacatttcactgtcacgtgaaatacagctgatatataccctttagcctcaataacttcctttagcattttgtgttatgcagatctactgaaaatggattctaccaattgtcatttgtatggaaatatccttaatttgccctcgttttttgaaagatattttcactaattctctaggttgatgggcactttttcttc

SEQ ID NO: 420 - DNA, 401 bp, Homo sapiens
agagatgttgtaactcagtgttttcatttctaattctcggatcatgaaaactaacaggcttggcttggtgtggtggctcatacctgtaatcccagcactttaggaggccaaggcgggtgtaacacctgaggtcaggagttcaagaccagcctggccagcatggtgaaaccccgtctctactaaaaatacaaaaaattagcygggcgtgatggtaggcgcctgtaatcccagctactcaggagactgaggcaggagaattgcctgaacctggaatgtggaagttgcagtgagccgagatcgtgccactgcactccagcctgggcaagagagagagagactccatctcaaaaaaaaaaagaaaaaagaaaacaacgggcttatttacagcaggagctgtggtg

SEQ ID NO: 421 - DNA, 401 bp, Homo sapiens
gggagcctgggcgggccagggtaacgaagagcccgccgtcggtctctgcatccgtcggctcacgtggccacggagcagcaccgtgttacagcaaagcggtcctgatccagaacccaagagagggttcttggatctcgcacaagatagaattcagggggagtccgcagtcccatgtggaagtgagtttattaagagagtaamgtggcgaaaggacggctgctccacagacagagcagggcattcccgaaactaagaggagaaaggcgcccacctggggtacaatcctggtgtatacggggagatgtgctctgctacgagcgttgatgataaagaattaattttcttaattagtatattttgcaagaatcaatatcatttatctttaaggcaaaattaggaat

SEQ ID NO: 422 - DNA, 401 bp, Homo sapiens
tgaggagccgaggccaggaagggttttgccagatgcctgggctggggccagggctcaggaccacccactgaactgcctgctcggcccaccctggcaagtgtgtgcaagggcccggtggtgccgacgaggagggccatggggaggagatgttgttgtcctgagactcccagccccacctgagggggaagagggtgggagagyaaggctgggagccacccttgggggctgtgcatgtgccccctgacattggaggacacaggccacgccacacctgtgccacccagggagtgggaaggaagcacgtggccgtggagaggccagcaggtggcaggaagggctgcaagcccccaaccacggggtcacacgtaggggacccagcaccccatgcaggagctgggctg

SEQ ID NO: 423 - DNA, 401 bp, Homo sapiens
ctcggcccaccctggcaagtgtgtgcaagggcccggtggtgccgacgaggagggccatggggaggagatgttgttgtcctgagactcccagccccacctgagggggaagagggtgggagagcaaggctgggagccacccttgggggctgtgcatgtgccccctgacattggaggacacaggccacgccacacctgtgccayccagggagtgggaaggaagcacgtggccgtggagaggccagcaggtggcaggaagggctgcaagcccccaaccacggggtcacacgtaggggacccagcaccccatgcaggagctgggctgtgccctgcatctgcacaggccggggcatgaactgggcatcagcaccgccctcccatggggacgaggacccatgcccgcg

SEQ ID NO: 424 - DNA, 401 bp, Homo sapiens
ccacccagggagtgggaaggaagcacgtggccgtggagaggccagcaggtggcaggaagggctgcaagcccccaaccacggggtcacacgtaggggacccagcaccccatgcaggagctgggctgtgccctgcatctgcacaggccggggcatgaactgggcatcagcaccgccctcccatggggacgaggacccatgccygcgtggtgcaggagctgtgcactgagcagccccatgggcaactccagcccgcagtgccaggagcagggccaggccttgtggcaggggtgcaggctggctcctggcggtcgctcagctctcggggataggtgaggacgcgtggagaggggatagcatgggcatcaaggccgaggcactggcccctcccagaatggcctcca

SEQ ID NO: 425 - DNA, 401 bp, Homo sapiens
ggatgtcacaggagaggaaaaggctttctaaccatatcttataatttagaagaacaaatacaagattgccaaaatttatcacttaaaataatgtatactcaggtctggcacggtggctcacacctataatcccagcattttgggaggccggggcaggaggatcacctgaggtcagcagattgagaccagcctggccaacaytgtgaaaccgtatctctactaaaaatacaaaaattagccaggcctggtggcacacgcctgtaaccccagctactagcgaggctgaggcaggagaattgcatgaatccgggaggtggaggttgcagtaagccgagatcgcatcactgcactccaggagcatctcaaatatgtgtgtgtgtgtgtgcgcgcgtgtgtgtgtg

SEQ ID NO: 426 - DNA, 401 bp, Homo sapiens
agagaagacaagccacagactgggagaaaatacttgcaaaagacttatctgatagaggacagtactgaaaatatgcaaaaaactcttaaaactcaacaataacaaatggacaatagattttaaaagtgagcaaaatactggaacgtaaatctcaccgaagaagttctgcacatggcaatacgtgtgtgagaatgttcagcrtcacgcatcatcggagaaatgcaaattacaccaacactgagacaccacacacacctactagcatggccaaaacccagggactggcaacatcaaatgctgacaaggatgcggaacagcaggaactcttgttcatcgctggtgggaatgcaaaatggggccgccgctgtggaagacagtttggcagtttctcacaaaactag

SEQ ID NO: 427 - DNA, 401 bp, Homo sapiens
aggttttggaatggttaaaatggctttaggtagtgagttttgtatggcttaaaaatcttgaaactgtagaatgcttctcatctacaggatgctaatgtctgttgggcagttgaagatttcttgcttcctacctgtatataaaatgtgctagggaagatacattattgggaaaagaataacttttgtccagaaagtattaawtgaggggctcaaaatatgagggaaccagtacgagtagaaaagagagaagtggggagttatacatacatacattttttttttcaggaggggtatgaagactgacttacaggtgcccgccaccatgcctggccaatttttgtatttttagtggagacggggtttcaccatgttggccaggctggtctcaaactcctgacctc

SEQ ID NO: 428 - DNA, 401 bp, Homo sapiens
cctgtatataaaatgtgctagggaagatacattattgggaaaagaataacttttgtccagaaagtattaaatgaggggctcaaaatatgagggaaccagtacgagtagaaaagagagaagtggggagttatacatacatacattttttttttcaggaggggtatgaagactgacttacaggtgcccgccaccatgcctggycaatttttgtatttttagtggagacggggtttcaccatgttggccaggctggtctcaaactcctgacctcaggtgatccacccaccccggcctcccaaagtgttgggattacaggcgtgagccactgcgcccggccaagaaagactgactttgtatgagaaaggatctctggtcccagaataaagagactggttgtgagg

SEQ ID NO: 429 - DNA, 401 bp, Homo sapiens
tggcctctggggcccttagatcagagattttccctcctctaaccttacacaggacctatgcccatgaacacagcaggaagcaatcccagaagacagaccctgcccttctgcacccccttaagattaaggggaaggatctcatctctgaagagaggggatgaggtaggaaggtggcaggacttgttttctggtcactgaccmactgaccaaaacaggatctgggctggatgggatgaagtggagacaggaatcaggggacggcgaggagggtgatccctggctgccatcattgctcattggcataagaccctcccaccagggccatgatggtttgcaaatgccatggcaatgacctggaagttaccaccttttttcattgcaacgacccagaagttgtcact

SEQ ID NO: 430 - DNA, 401 bp, Homo sapiens
gcctctggggcccttagatcagagattttccctcctctaaccttacacaggacctatgcccatgaacacagcaggaagcaatcccagaagacagaccctgcccttctgcacccccttaagattaaggggaaggatctcatctctgaagagaggggatgaggtaggaaggtggcaggacttgttttctggtcactgacccamtgaccaaaacaggatctgggctggatgggatgaagtggagacaggaatcaggggacggcgaggagggtgatccctggctgccatcattgctcattggcataagaccctcccaccagggccatgatggtttgcaaatgccatggcaatgacctggaagttaccaccttttttcattgcaacgacccagaagttgtcacttc

SEQ ID NO: 431 - DNA, 401 bp, Homo sapiens
tccatccatccatccatcatccatccatccatactatccagctacctacctatcatccattcctccatccacccatccattatccatacatccagccagccagccttccatccacccatcctatctatccattctatctacccacctatcatccatccatcctatccacctacttacctagcattcatccatccatccatycatccatccatccatccatccatccatccaatctattcactgtctccacctaatttctgacatctcatctttgtaatttgttctccagtgagcatcttttcacatgcactggtgtttgttttgcctcctcctggggctgcctattcttcctagctggagcactaggctctacgttgtccttgtggtatttttctttgatt

SEQ ID NO: 432 - DNA, 401 bp, Homo sapiens
cacaagtgtgtgcacacacagtaaacacatgcgtgtacacacaaacaagtgcacacacacactaactaaacacacatgcgtgcacacacaaaacaagcacatgcacacacatggtaactacacacatgcacgcacacatacaaaacaagtgcaggcacacaataacatgcacacacacaaaacaagcgcaggcacactaamcacacatgcgcacgcacaagtgcacgcacacacacggtaacacacatgcgcatgcacacatggtaaccacacacatgcacacacacacaaaacaagcgcaggcacacgcggtaactaaacatgcatgcacacacacaaaacaagcgcacgcgcacacagtaactaaacacacatgcgtgcacacacacaaaacaagcaca

SEQ ID NO: 433 - DNA, 401 bp, Homo sapiens
ttcgagaccagcctgaccaacatggagaacccatctctactaaaaatacaaaagtagccgggcgtggtggcgcatgcctgtaatcacagctactctggaggctgaggcaggagaatcacttggacccgggagtcagaggttgtggggagctgagatcgcgccattgcgctctagcctgggcaacaagagcgaatctccgtmccaaaaaaaaaaaaaaaaaagccgacaataagttactgggtaagtttcttcttcccataaattgttcttagaacttgttgcctccctgggcagacgccatgaagtgtgataaccagcactaagagaaagtctcaggaagttccccggtctgaccctggggccacggcagcctctgcccacagcctgcttcagtccacctc

SEQ ID NO: 434 - DNA, 401 bp, Homo sapiens
tgctgagactgggcatcttgtcttgttaacaatcttggggattgttagtacccagtgcccagtcttgacatgagctgtgggttttcacagatgccctgaaaaaggatgagggtgttcccttctattgctagtttactgagtagtttttatcattttggccaaaaagcttgctcgcgtctattgagattatcttaaaatttwcttcttaatcctgtgaatatggtgaattgaattggttcatattctgattttaaaccaaccttgtttttctggataggcctcacttaaccatactacacgattgatttattaccgaatttaaattgataaatttatgttaaagatgtttgtgtctatgttcataagaaatattggccgtagtttccctttcttataatgtc

SEQ ID NO: 435 - DNA, 401 bp, Homo sapiens
agtttctttttctctgctaatatgtcccatctttttatttattgtgagtgtgttttctttgctctaagcatagctggaatggccgccttacagcccacatctgctgtctccgcactggcgcctcagggcttaggttggttggttgtcctgttctttgagattgggtcacgtttctctgttcttcatgtaggacgtaggagsctctggggtgcacgttctcgcagtgaatgctggtggtttccctttggtgctgtctggttgggtctgactcacatgtgaactctgtctcctgggtggcagcctctgttttggttcagttcagtccttggctaagctgctttgggtcactccacacctggtgatcctggtgttaaccagtgacgtgacatggatagagctta

SEQ ID NO: 436 - DNA, 401 bp, Homo sapiens
gctggctgtccatgccccctttgccacctacttctccttgctgtggagggcagagctaaggctccctcctcgtggcgctggggagttctgatggcggtccagggccctcgccaccatcacctgagcgctcatgttgcccacacagccggcctcgccgccaaagcctttctccctcggacttcaggtggagaggctgctgcsgtctctccccctccctctgggctctggcctctcctccttgtcttggctgatggctccccctcatccagctctgaatgtggctccctccacgccccttccctctcttctctgcacaccctcgtcctgtgtgctctggaccacgcctgtgccttacacactgcttgcccatgggtgacgcctgcatccgcgtctgacctcct

SEQ ID NO: 437 - DNA, 401 bp, Homo sapiens
ggtgctgctggtcccgctggtggaggaactggcagggtggggagcctttccccctgggggacacatcctatgaggacagcggctcccaggaggcctccagaggctgggaggcctttcaacgcgaggatcttactggccacaggtgctgctcaaacgaacgctccgaaagtctcagcaggtgagagcgcaagtctcagtgcyggaaactgctgtgctgggggtgaggaggggtggacccaagggcagccaagggctttgtgggggcacctggggcccagggcacaggaagagccatgccccacatccagtgcccccaggtgtaaccctgctcctcatggcccaagaggttttacgtgtggatgggttttaattgggtgtctggaagcaggtaaagttgtgtc

SEQ ID NO: 438 - DNA, 401 bp, Homo sapiens
gaacttagagagtgcaggatgttcccagtgttgtctggatttccaggctttgctttgcaccttgcctccctctggccctcttgctcctggcctggctccggctgcccctccatgcagcgccgccctctgtgcaaggctctgagtggggctgcctggaagaggagcccaaggcctcctcacccctcctggagccggtagctsggggtcccacccaacatcgccctgtggcatctgcccaaacgccccatgggcatcctgcctccctgcacctggtgaaagttctgtgggaggcgctctccattcctgccaggtctttatcataccatgctctgggcaatgtttttgttaaataagataacatgagccctctccaagttacctgtcctgcagggtgcagggtg

SEQ ID NO: 439 - DNA, 401 bp, Homo sapiens
taacagcatctcagggcaaaacaatcgctcggggtacagatcaaaatggagtttcttacgtcctccttttctacacagacacagtaccagcctgatctctcttttccccacatttacaaacgccacagtgaagaactttgttgcaaacacatgcaggctgtgaaaagaactctgtgcctaaagtcctttcatttctttgcyggagtttttttgaggcagattctagaattgctgagtcaaagggcaaatgccatgtagttttgttagacgctgccaaatcctcttccatgaggctgcaatattctgcattccacagtgacgtatgagagtccttgtatcccacagcttcataaacaaagcatgacggcacatctgcacgtttaccaatcacacaggtgaga

SEQ ID NO: 440 - DNA, 401 bp, Homo sapiens
aacagcatctcagggcaaaacaatcgctcggggtacagatcaaaatggagtttcttacgtcctccttttctacacagacacagtaccagcctgatctctcttttccccacatttacaaacgccacagtgaagaactttgttgcaaacacatgcaggctgtgaaaagaactctgtgcctaaagtcctttcatttctttgctrgagtttttttgaggcagattctagaattgctgagtcaaagggcaaatgccatgtagttttgttagacgctgccaaatcctcttccatgaggctgcaatattctgcattccacagtgacgtatgagagtccttgtatcccacagcttcataaacaaagcatgacggcacatctgcacgtttaccaatcacacaggtgagaa

SEQ ID NO: 441 - DNA, 401 bp, Homo sapiens
ctgatgcttccgagctccccttcttactcaccatggggactgtgtaagagtactcagtgtcctccagcttagaaccagagcccacacgtgtcccattgttaccctgatgcttccgagctccctttcttacacaccatggggactgggtaagagtacttgggtgtcctccggcttagaaccagagcccacacgtgtcccatygttaccctgatgcttctgagctccccttcttactcaccatggggactgcataagagtactcggtgtcctccagcttagttccaagttttctgtcgctccggtgacccttcgacctggattcgagcccccatgtgtgggcgccacttgctgagaccagctcggttgtggagaccctaacccagcggcactagaggaattaa

SEQ ID NO: 442 - DNA, 401 bp, Homo sapiens
cctccacccatagcccctcgaaaccactgccctgctccccaacacggtagcactgtcttctccaagatgtcatgcggcggcatccctcggcctgtgcgcaccgaaactagcttccttcaccgggcatatggcctgggagacctggggcatttgggtgcatccttccactgctggttggtgccccctgtgtggaagcatcckcgttggttcccgcctcctcctgccgatggacattttgttttcttccagttattggcaatgaggaatgaggcctaaacacttgtgtgcaggtttgtgcatgcatgtttaagttttctcttgggggacgtttcaggagcggggttgctggatgacagggtaaagatgtgcttaacttcataagaaactcccggaccactttc

SEQ ID NO: 443 - DNA, 401 bp, Homo sapiens
actaaatggcatattttttagggatgtgcagatacgtggtgaaagaaacgtactatgaagaaaagtgtgggaataataaagactaaagcaggaactgattccctctgcaggagaagggaagggacccgggactcaggcagggcctccagggagcatccacagttatgtttcttgagattctactccctaagcttggtggargtcctctgtgtccaatgtgtcaatattctttataccttacccatactgtacaaatgctttatttctattcaacatttagaaggcagttataaacaagatgcattcaatagcaaggtggcagatgaacatcaggaaggaacatccatgagcttccatccacggaacctcaccatggatacgcttgtgatcaagggcctggt

SEQ ID NO: 444 - DNA, 401 bp, Homo sapiens
aaaaataaacatccagatgaagatgctgtggaagctgaggggcatgaggtaaaaagactcacgtttgacaaaaaaggcgaagtcagagaaacagccattcaagcgacttgcagcgaaatttcttcggttatggtagaagaaacagaagcatcaccttcatctcaggataaagacaaagaaagccgttgtacccggcagcaygtacagaagaggatgaagaagaggatgaagaggaagaagaatatattgagagaccatctgtaaaagggaggagtaagaagatcctcaaattcttgtattcattgctttcgtgaaagaattgtacataatagaactccttgtaatgccgacattgggcttttctcccacctgtatgtagttgttgctgaatttcaggggat

SEQ ID NO: 445 - DNA, 401 bp, Homo sapiens
tttgagggccatgcagctggagactcagtggtagccgatgttgcagtttgagggccgtgcagctggagacccgggtgggaaccgatgttccagtctgggagccatgcagctggaggcactgcggggagcagatgttccagtttgatgttcccccctgggtctccaggtgcacggccctcaaactggaacatcagctccccrccggatctccagctgcacagccgtcaacatcagctcctccccgagtcctcagctgcgcaaccctcaagttagaacatcagcttctccccaagtcttcagctgtgcgaccctcaatctagaacatcagttcctctctgggtctgcagctgcaagaccctgaaactacatcagcttctctctgggtctgcagctgcaagacc

SEQ ID NO: 446 - DNA, 401 bp, Homo sapiens
aaaaattagctgggcgtggtggtgcatgcctgtaatcccagctactagggaggctgagacaggagaactgcttgaacccgggaggcagaggttgcagtgagccaaggtcatgccactgcactccagtctgggtgacagagagagaccctgtcaaaaaagaagaaagaaagagagagagagagaaagaaagagagagagagraaggaaggaaggaaggaaggaaagagaaacaaaggaatgtttgaaatcaatgacataagcttgcacctaaagactcaagtaaaggaaatgtagcttgaactgaaagtgagtagaagagaggaagtaacaaggataaaataaaacatcaatgacagaaagtagaaaaaaaatcactggaaagaaaagaaggttctttgaaa

SEQ ID NO: 447 - DNA, 401 bp, Homo sapiens
catcttccacacccctcctggcggtgacttgggttcctcacaactcgaaggacaccagttctgcctgtgtgtggcaggactcagcccagcaccatgatcctcctggacacccagccaggaagcacagggtgcaggtggcccaagctctccccatcgggccgtcgcttcaagccaggcagctggctacactgcctagggagmcttcctgacggtgtgttcagaaacagcaatgccagccggcaaggacttggtgtgcagccgtgagcactggccttgtgagtgcgggggcttggcacgctggcactgtgcgtacctgcaaggcctgggtctggacgggcgggggcggcatggtctaggcctccttagagcagccccgaagccccagtgctgtggccgaatga

SEQ ID NO: 448 - DNA, 401 bp, Homo sapiens
tttattccttcccattctctctcttaaaacaaatgcttgggaagacagatgtgagctgtaagattccgcggctgtgttagaggaggcgccggggcctgggtgcagaacgagtctccaagtgccgcgtgagcgagggtgggtggctggagctcccgggccagcccgggcggaggcgctgcctccagacccagcctgtgtgcrgaggggctggcggagctcggacgccgggctcgctggcctttgccgatggacctcgggtgtgtccaggccgggcgagggccggcagctccagtgaggcgggacctcactctatctgtgttgtgcatcctgttcgagtttctgggtcccgatcttgagcaagccaactgacctctctgatgttggtttgctcagatgtaacg

SEQ ID NO: 449 - DNA, 401 bp, Homo sapiens
gagatggggggcccagggagggacaggaagaagctggtcccatgctgcccacgttgtgacgtttaccagggcatcctccagcctaacagctctaaggtttggttcaccatataagcagctggcaggctgagcacaaccttccttagccctgcactcctgtgtgtttgggcaggtgggctcataggtgccggagatgtttcsggcagcatgctgcgcgttacactcaccctggcactggggagtcggagctgagccaggcggagagcatccatacagggaggcacagaacaaggcagcagcgcagtgcccgccgttcactcctgcctaacagatgtccccaggggcagcaccttcagatggcagatggcagccatgtccttagctcacaactctgtggtcag

SEQ ID NO: 450 - DNA, 401 bp, Homo sapiens
gtgctttgggagtcctgtgggactgcagaatgagcacgttcgctccatcgtttgacgacaaaggcacccggtgtccctgagccgagcacgccccgtccacttggtgggggcattagcctgtcggaaagctgcctggtccaggggggctttcagcccgcaggacgccgagagctgtctgtaaacctctccatggttgtgtgrgcctcgggccgaggcagcctgcagtgaaggaagatgttttggagaagttgttggggggctgaatccctaaacgcaggggacacttcacgcccgtggaatttggaaaaccgaggcagggagcagggaagggtgtgggactcgggaaggggagcagctggtgctcctttaactcgcccggtggcagttcagggtgtttccat

SEQ ID NO: 451 - DNA, 401 bp, Homo sapiens
gggagggagacgaaaggcggatggaagaggggcccccggaccccgcccctcgaggcttgtgtgccgccctggctgcggcgttctgggcgacgggaaacagagtcaggacacagaccagggagggccggggcagagccagggcacctgcgggcgaggacggctgggtgctgggtccccggcgtcagggcgcaatgccgcctkctgggcgtcaccctttcccagggctgctgggacacgtggccgcagctgcttggctcaccacagcggaagtcggtctctctcatcctggagtccgcgcggctgagggcgaggtgtccttccgggggccctgaggggctctgtccccggcctctcccctcggcgcccctgcccggccaagcattgcgccaagcccaccccca

SEQ ID NO: 452 - DNA, 401 bp, Homo sapiens
ccccaagctgagagtgtgcacaccgtttcccagccccccagagactgccctgcacgggagcttacacacccattgcacaggtggcctggccaggagcctttcccggatttgctgagaaaggtgcgcctttgtcatctccctccatggggacctccacccccttgtccctctccttcctccacagggacctccaccccctcrtccctctccttcctccacagggacctccgcccccggtccctctccttcctccatggggaactccgccctcccctccctctgccaccccacctgggcccctcgccctcccctccctcacactcaggccttgggctttgcgttgctgtgctgacagctccctgctggctgccctgctgtcgtagcaagtctgctctgctcac

SEQ ID NO: 453 - DNA, 401 bp, Homo sapiens
caatggagggccaggcagccgtttaaaagaacaaagtagaactgcacacactgctggggcgggatcgtcatgacagccaatgagctacagagaagtcaggtcccatctaaaatatatttttacaaatatattaatattaataaatatacttaaatatatccaaaatttatatatatgtatatatacacacatatatgtgtrtatatatatatatatatatatttttttttttttttttttgagacaaggtctcattctgccacctaggctagagtgcagtggtgtgatctcggctcactgcaacctccacctccctggttcaagcaattctcatgcttcagccacccaagtagctgggattacaggtgcccttcgatgcttactaaggtcccctgtggagg

SEQ ID NO: 454 - DNA, 401 bp, Homo sapiens
gcaatgtgagattttttcatttaatgtgaacagacacaataacggttcacatttattgaaccttttctgtggtgaggtattgtgccaaatcctctaaatatattccttcatctaattcttacgaccgctccatgtgagaagtatagccacctccattttacagatgaagaaactgaggttcagactggtgaagtgacttgyccaaagtcatgtggacttggggcaggagcctggacatgagcccagcctgcaggcttgcaggctcctaactaccagcctgtgcatcacctctcactaaaaacaaaacccaagccacgtaccccgcaaacgcttcatatctgacttgtgtggaggtgacaaaaccgattcctgaagctttgtgtctggtgggtgtgagacac

SEQ ID NO: 455 - DNA, 401 bp, Homo sapiens
gaggccaaaatggaagctaaacagaggagaaggtgactgcccctgtacggtacacctcatccctttcttccgaggttagtgggcaaaagaggacttcccatcttcaggaataaaaaagcaccaccaccactctctcttattaagcaaaaagaaaagctcttgttttatttaattaattaatttatttatttattattattytttttgatgtggagttttgctcttgtcacccaggctggagtgcaacggcatcttggctcactgcaacctccaccaggatcctgggttcaagcgattctcctgcctcagcctccagagtaactgggattatagatgcccagcaccacacccagctaaattttttatttttagtagagatggggtttcaccatgttggccag

SEQ ID NO: 456 - DNA, 401 bp, Homo sapiens
aagtaacaagaagaacaaaaaatgagaaaacgagttacagtttcaaacgtggagaattcacattaagaggaaaataatgtaataaacgttttagacatatccttgtaaacatcacagatcatttaaaagtttatttctggtcaatatttccacagatatttttaggatggaaaaatactttcaacggctacgaacaagackcctcgaaccttaggggaaaaattgcattttaaaagtgaatattgctttagcaaattcttcggctatttttcttttgtttaaatgcctttacctgtgttcattttgtatgcctaagaaacctagaaaaatagggtactgttctaagtgggaatgattctgtggaaacagtgaattagcccaacaattggggagggtttaaa

SEQ ID NO: 457 - DNA, 401 bp, Homo sapiens
tgtgttcattttgtatgcctaagaaacctagaaaaatagggtactgttctaagtgggaatgattctgtggaaacagtgaattagcccaacaattggggagggtttaaaaaaagcccccaagaaccttataaataatggcaaaaccttttaagtccgacttaaaagagtgaggagtaagatggcgcagttataccttcacarccctctgaccccaaaattcacgtgtgttcaccgggaagagccgggagagctggttatttttgtttgtttgtttgaaaacacagctcaggttcagaggttccctaacacggagaaaggcaccggtcccgaagagggcctgcggccattgtgtcccaagcgcaggtgtcaaacgccggccgcggcgcccgcagggtccgtcc

SEQ ID NO: 458 - DNA, 401 bp, Homo sapiens
gatgaaagaggagggtctctagctgtcattgttttgagggatgagaagttgaaagagggattctgtgggggagaggtggaggatacccagcagagaatagagggcagggtttggagtagtcaaacagaactaaacttgaacccaagatctgcttcacctgctgtgtgacttttggcatgttacataatctttctgatcctyagtttcctcagtgcaaaatgataatgacactctactgtgccggtgtgaagattaggtgagttagtaaccacaggttcctagaatatggtgggctctcacgtatttagaaagaagaggcacatcttgataacatgctgagaactgaggcaactgatgtctttaggctcttatagtcccagcatgtttttaagagctactga

SEQ ID NO: 459 - DNA, 401 bp, Homo sapiens
aagaaaagcaaagggtagttcattcctcgaagccagcttcctctatgagttctatacaaagcctcagtagagtgggtcccattagcaaccaagttgaacaacttttatttgctgactgaatatagatacacctgaattgttgactgcttttgtaactaaacactcctctcctgtcttccaacgagtggtcatttttgtccrcaacttgaccacagcaatccctggggccctagctctactctcaataaagagttatggctgtgtgttttgaatgacaccttaggacccatcctgcctccacctccttctccataaaatagaaacctaacttgcccctccaagctctgaaatgctgaaacttaccaactcccttttctccccgctatttcttccttccgtgg

SEQ ID NO: 460 - DNA, 401 bp, Homo sapiens
gcttctctcccaggctgtcaggcggcgcgcagggaggaggtgcagacgcggccgccaccgccaagcgcccagcgcgcggctctgaacttggcgccagagccctgaggacaccaggccccgccttcctggccgcacacgccttccgggcagggcttgtgggtgggcggccccggcgcgggagggtgccggggccggagaacycgcaacccggtagaaagcctgtgatcagctgggcagcccagtcctccccgggctcagctcggcagacagccctctgatggctcggactgggccgggcgccggcgcctgcgggagggcgagggcgtcctttgaaggcggctccagctctcgccgccgccctctggctctaggctcccctacctctcgctcctgggccctgg

SEQ ID NO: 461 - DNA, 401 bp, Homo sapiens
cttgtgggtgggcggccccggcgcgggagggtgccggggccggagaacccgcaacccggtagaaagcctgtgatcagctgggcagcccagtcctccccgggctcagctcggcagacagccctctgatggctcggactgggccgggcgccggcgcctgcgggagggcgagggcgtcctttgaaggcggctccagctctcgcygccgccctctggctctaggctcccctacctctcgctcctgggccctgggggccgcggcgatgcgccccgtgcctgtggctccctcttcgcggacgacccccgtggcctcccagccctccctccccgcgcgacctggcggccccgctagcggcacctccccagcgcccaagtagcccagatcctccaggagcgccccgccg

SEQ ID NO: 462 - DNA, 401 bp, Homo sapiens
gtgggtgggcggccccggcgcgggagggtgccggggccggagaacccgcaacccggtagaaagcctgtgatcagctgggcagcccagtcctccccgggctcagctcggcagacagccctctgatggctcggactgggccgggcgccggcgcctgcgggagggcgagggcgtcctttgaaggcggctccagctctcgccgcsgccctctggctctaggctcccctacctctcgctcctgggccctgggggccgcggcgatgcgccccgtgcctgtggctccctcttcgcggacgacccccgtggcctcccagccctccctccccgcgcgacctggcggccccgctagcggcacctccccagcgcccaagtagcccagatcctccaggagcgccccgccgcct

SEQ ID NO: 463 - DNA, 401 bp, Homo sapiens
gagggtgccggggccggagaacccgcaacccggtagaaagcctgtgatcagctgggcagcccagtcctccccgggctcagctcggcagacagccctctgatggctcggactgggccgggcgccggcgcctgcgggagggcgagggcgtcctttgaaggcggctccagctctcgccgccgccctctggctctaggctcccckacctctcgctcctgggccctgggggccgcggcgatgcgccccgtgcctgtggctccctcttcgcggacgacccccgtggcctcccagccctccctccccgcgcgacctggcggccccgctagcggcacctccccagcgcccaagtagcccagatcctccaggagcgccccgccgcctccgctctcctggcccccgctccc

SEQ ID NO: 464 - DNA, 401 bp, Homo sapiens
tggcccccgctcccctggccccagctgtgaccgcgcagcactttgccaggaaggggggctccgcgacccttacaacacagaggtgtctgggggcttaactgcccctggagggtgggagggggccagggcagaagggccgtggtctggacggaatgactgtggggtgggtggagactgcgctgggactcggaggacctgggstttcccctgcagtgggggagtgagggtagatgggatagagtctcaggtaatcgcagttcttggtgctgttgcaggcaagtgtgcatgggtcacattgcttgagcgtagggtgcaaagcagcttcgtttggagaagggagaattcttcaagtactgaaaggaaaaccaccctgagtccctgctccctcattgattaattcc

SEQ ID NO: 465 - DNA, 401 bp, Homo sapiens
tgcgcacctgtagtctcaggtagtgttggtgtgcacctgtagtctcaggtactcgggaggctgaggcacgagaatgtcttgaacccaggaagcagaggttgcagtaagccaagatcgcaccactgccctccagcctgggcaacagagtgagaccatgtctcaaaaaaaaaaaaaaaaaaaaaaagcagcaataaaaactgrctatatactttcaaaaagagacatatctaatacataaggacataaaaaagtttaacataaaaggatgggaaaagataacggagatatggccgggatgctggggaaaccaaaaggaagtgggagtcttcagcacagagaaagtgtgaccaggggttaagaggatcagttcctaatcagctgcattttccaggaagccataa

SEQ ID NO: 466 - DNA, 401 bp, Homo sapiens
ggtgaaaccctgtctctactaaaaatacaaaaattaaccaggcctggtggcacgcccctgtaatctcagctactctggaggctgaggcaggagaatcgcttgagcctgggaagcagaggttgcagtaagccaagaccatgccactgcactccagcctgggcgacaaagtgagtctccatctcaaaaataataataataatmataatcatcatcatcatcatcaataaataagtgttagtctgctctgggtgtcataagaaaataccacaggctgggggacttaaacaataaaaatttatttgctaccaatcctggaggctgaaagtctatgatcaaggtgccaccaggattggtctctgatgagactcctcttcttggcttgcaaacagagaccttctcat

SEQ ID NO: 467 - DNA, 401 bp, Homo sapiens
gaaaccctgtctctactaaaaatacaaaaattaaccaggcctggtggcacgcccctgtaatctcagctactctggaggctgaggcaggagaatcgcttgagcctgggaagcagaggttgcagtaagccaagaccatgccactgcactccagcctgggcgacaaagtgagtctccatctcaaaaataataataataataatmatcatcatcatcatcatcaataaataagtgttagtctgctctgggtgtcataagaaaataccacaggctgggggacttaaacaataaaaatttatttgctaccaatcctggaggctgaaagtctatgatcaaggtgccaccaggattggtctctgatgagactcctcttcttggcttgcaaacagagaccttctcatgga

SEQ ID NO: 468 - DNA, 401 bp, Homo sapiens
gcatgaataggcacttcacaaaggagggaacatgaatgacccaaaatcctgtgaacagatgaacaaccctttagtaattaggaaactgcaaattaagtcaatacttttgtctattgattagacatcaaaacataattagaaatcaaaaaatataaaatatggcactaccaagagtcatgaaggatgtgaagaaagaaaacmtaatatgctttcggaggaattacaccaactttacccattctaaaataccacttgtttaaaatgatccattattttaaacaccactccaaagcaagacaaaaaacccccaaagtccactaacccagtgctttttactacttgtaattttttttttttttttgagaaagagtcttaggctggaatgcagtggcgcaatcttg

SEQ ID NO: 469 - DNA, 401 bp, Homo sapiens
ctgtgggtttggcagagagactggagtgggtgtgaggtggtaaaaagcaagtctatgggggaggactgtggcgggtgacccagacggggagccgggtttgggcatggcctcttctgggtatggatggacctggcctcctcccctcctgagctgggcaggcagggctctcagagctccggccatggcagggactctctgccygcagtgggaagccagccttcagggtccatgctctgcccagccttgctgtgcaggctcagcctgtcacattggattagcctgaccctaagtccccagcctgtcccagtcctgccaccagagccagttctggctgccccagcccattttgtctgttttaacagcagctgacaggctatatcccccagccagtctgcagatca

SEQ ID NO: 470 - DNA, 401 bp, Homo sapiens
tccagctgggctgtgtgaaaaggacaagagaagagccaaggagtttgcccccttgcacacaccttagtcttcctcctcccttcctcgtttcccctctgagtgacttgcatcccttgagtgtgtctcagatgatgcagaaggctgtccacagccgctgactcccctccccactgtgtgcctgcagctcttgtcaggaaaagkcagggttggtggggaaaggcgttgaggtggccgtggggtctcccgccccggtgccactgacccggagtcctgtattcctggtcccagagatgccggtagggctgcccacaggctctgcccacaggagagcccagttgctcctccccctgcctgtgcgacaggcctgtgcttgtggagtcctggctggagctgccctcagg

SEQ ID NO: 471 - DNA, 401 bp, Homo sapiens
ggaaaggcgttgaggtggccgtggggtctcccgccccggtgccactgacccggagtcctgtattcctggtcccagagatgccggtagggctgcccacaggctctgcccacaggagagcccagttgctcctccccctgcctgtgcgacaggcctgtgcttgtggagtcctggctggagctgccctcaggcagaggtagtgcrggctgctagcgggtgagggttcagacagcctgatgagaaggggcctccccctctcagctcctcctttgtaaaatgggtgctgggctgagacctccctcactctaggggtctctgggtagcaggtgcttggctcttggcagcctctcccctggaggatgaggtggaggcacctggacatgccctggagtgatcagtgctac

SEQ ID NO: 472 - DNA, 401 bp, Homo sapiens
gtatgtggcataagctggggtgctgtggaagtgtgtgggaagggtacctggtgtgtggtgtctagtgtgtgtttgttgtgtggaggagtcgggagtggactgcttgtgtgtggtgtgtgtgtatgggatgtgtggtgtgtgtttgctgtttgtggtgtatatgtgtttactatttatgtgtgtctgtggtgtgtgtgtgtwcagtgggactagagtgtttgggcgtgttgtgtgtgtcatttgtgggtgtctacatggggatatcttgtgcgtgtggcatgtagggaaggaaatggtgtaatggcatgtatgtatagtttgtgtgtgcattcagtgggaggtgggtgtgtgtgccatggctgtgtgtgtatggtgtctgtatatgtgtttgctgtttatgt

SEQ ID NO: 473 - DNA, 401 bp, Homo sapiens
gtgtgtatgggatgtgtggtgtgtgtttgctgtttgtggtgtatatgtgtttactatttatgtgtgtctgtggtgtgtgtgtgtacagtgggactagagtgtttgggcgtgttgtgtgtgtcatttgtgggtgtctacatggggatatcttgtgcgtgtggcatgtagggaaggaaatggtgtaatggcatgtatgtataktttgtgtgtgcattcagtgggaggtgggtgtgtgtgccatggctgtgtgtgtatggtgtctgtatatgtgtttgctgtttatgttgtgtgtgtggggtgtgctgggtgtgtgttgggggagtggggagtgtgtggtgcatgtgtgatgtgtgtggtgtgtgtatgggatgtttggtgtgtctttggtatttttggtgtat

SEQ ID NO: 474 - DNA, 401 bp, Homo sapiens
tgtgtgtctgtggtgtgtgtgtgtacagtgggactagagtgtttgggcgtgttgtgtgtgtcatttgtgggtgtctacatggggatatcttgtgcgtgtggcatgtagggaaggaaatggtgtaatggcatgtatgtatagtttgtgtgtgcattcagtgggaggtgggtgtgtgtgccatggctgtgtgtgtatggtgtmtgtatatgtgtttgctgtttatgttgtgtgtgtggggtgtgctgggtgtgtgttgggggagtggggagtgtgtggtgcatgtgtgatgtgtgtggtgtgtgtatgggatgtttggtgtgtctttggtatttttggtgtatgtgtgtttgctatttatgtatgtgtgtggtgtgtgggtgtgtgtgcttcgggtgtgtggt

SEQ ID NO: 475 - DNA, 401 bp, Homo sapiens
tgtatggtgtctgtatatgtgtttgctgtttatgttgtgtgtgtggggtgtgctgggtgtgtgttgggggagtggggagtgtgtggtgcatgtgtgatgtgtgtggtgtgtgtatgggatgtttggtgtgtctttggtatttttggtgtatgtgtgtttgctatttatgtatgtgtgtggtgtgtgggtgtgtgtgcttcsggtgtgtggtgtgtggtgtgatgtgtgtggtatagggtgggttgagtgtgtttgtggtccagtgtgcatgggtgggtgggtgtggatgtgtggcccacatgagtgtgaatgtgagtgtctattggtgtggggggagtatgcagaggtggcagtgtgagtacttgtgtacaattagtgtgttgttacctcacaacaataag

SEQ ID NO: 476 - DNA, 401 bp, Homo sapiens
tgtgttgttacctcacaacaataagtttaaccctaaagcaagaaaagacatgtgtgagaagctcacaggaaaacactagcaaatggcatggagcagcagctaacccagaccacagggcaggagcagccagggctcctcccaggaaggccaggcaggtgcagaaggtgaaatagctgtgaaatttaccacatcaacaaaacrggaaacaggaaccaagcggttatctcagaggatgaagaacaagaagtgggggcgtcaacatgccacagcaccctgggagcagaaggaaacctccatagcccgacaaaggtgtccacacaaacaccctcggctcacctcagtcccacaagatcaatcccagcatctgtccctactgtctggaattaagggtgacctcatgc

SEQ ID NO: 477 - DNA, 401 bp, Homo sapiens
aattaagggtgacctcatgccatctgggtacccagcatgggtggtgacagccttgtgtggtcctctgtgatgacccctgtctgtcctgatctccacctgtgcgcactgggactccgcatggccggcatttatgcaaatgcggctgctggagaggaagagagccctgggtgtcctccctccatggggacagcacgagactgsgtgctgctggcccagagcagcagctgggttctggcctcccagttcctcactgcagggtgccctggcagtaggctgtgaggacagacacaccgtccatgtcagctctgctctccatgggcacacgaccaggagcaaattgtccaatgtcagcgagctccagaggtgcctcccatgaacagaaaggatgctgtcaggcctgt

SEQ ID NO: 478 - DNA, 401 bp, Homo sapiens
tgagctgtgcagggactggcacagggctgggagctggtaggtcccagaaacaggagctgtgctgacacttccatgattggtcctgggctctgaccccactcacctctccccgatgcagagctgaggatgggggtggagtagagagagctcccctcccacttggggtctctttctgctgctcctggtttaaagctgctcagycgtgcagccagtgtgctagctgtgctcctctgacgatgtcccctggacatgcacaggctgtggcgtctgagcagtcctgtcaaccccaggtgaggatgtgagtcgtcactcttccctggtggcagctgagttgaggtgacaggtgccctctgcccaccagctcagccctctgcctctgctcacgtggcccggcagtcaca

SEQ ID NO: 479 - DNA, 401 bp, Homo sapiens
cctgtcagctgttggctgacttgggcaaagctgccccacatggagggtgacagtcacagcagggaaaggccaaggcaggaacctggaacctaaatgcaacagaataagccctctcagcaggctgccaccaagaaaccaggagggctccagggagcccaggaaggaggccagggagggaggcacgcagcccagactcccccrttcagtcctaggcctcatatgcagcctggcagaggcctctgctgggctcactgctccaggccagtcctgagcccatcccccacacatggctggagggttttggcaggagcctcccttctaagcctggcaggtgtctgaggctcttagcagaacccccaggccctccaggagccacttcctgttctgggcgccctgtgcag

SEQ ID NO: 480 - DNA, 401 bp, Homo sapiens
ccctctcagcaggctgccaccaagaaaccaggagggctccagggagcccaggaaggaggccagggagggaggcacgcagcccagactcccccattcagtcctaggcctcatatgcagcctggcagaggcctctgctgggctcactgctccaggccagtcctgagcccatcccccacacatggctggagggttttggcaggmgcctcccttctaagcctggcaggtgtctgaggctcttagcagaacccccaggccctccaggagccacttcctgttctgggcgccctgtgcagaggtgggaccaggtggggcgtaggcaccctgcaagccctgcctcaccttgaagggccgagcccacctgtccttcacagccctgttcatagggcacctcatcctggaag

SEQ ID NO: 481 - DNA, 401 bp, Homo sapiens
atgcagcctggcagaggcctctgctgggctcactgctccaggccagtcctgagcccatcccccacacatggctggagggttttggcaggagcctcccttctaagcctggcaggtgtctgaggctcttagcagaacccccaggccctccaggagccacttcctgttctgggcgccctgtgcagaggtgggaccaggtggggygtaggcaccctgcaagccctgcctcaccttgaagggccgagcccacctgtccttcacagccctgttcatagggcacctcatcctggaagccttccctgatgaccagcccctctgactccctgcagcccaagggcttggtctgtccctctcatcagtcacttgtcacccaggatgtatcatgacttctgattgtctatgtc

SEQ ID NO: 482 - DNA, 401 bp, Homo sapiens
cttggtctgtccctctcatcagtcacttgtcacccaggatgtatcatgacttctgattgtctatgtctaagcataattttcttctttagttatttggttttcttccatcctttattgttgaaattttcaaacctataggtgagtggaaaggattgtgcaatgaacacaaacacacctatatccaccactgggattttacartgaatgtttgcccatgctcctttatctctctctctctctctcttttcatccatccaccttgcaaaaaagggaaaaaaaaatgtttgcaatacgtagtcctaacacagcctagaatgtaatttgcaaccaccccgattatgaagctttggggcagccctcagtcctgctcagggtggccgagttgggctgaccaaggggtg

SEQ ID NO: 483 - DNA, 401 bp, Homo sapiens
agcataattttcttctttagttatttggttttcttccatcctttattgttgaaattttcaaacctataggtgagtggaaaggattgtgcaatgaacacaaacacacctatatccaccactgggattttacagtgaatgtttgcccatgctcctttatctctctctctctctctcttttcatccatccaccttgcaaaaaarggaaaaaaaaatgtttgcaatacgtagtcctaacacagcctagaatgtaatttgcaaccaccccgattatgaagctttggggcagccctcagtcctgctcagggtggccgagttgggctgaccaaggggtgcaggccacaggcccagttgctccctcccgggcacactgagcccagtgtccggctggccagggccctttg

SEQ ID NO: 484 - DNA, 401 bp, Homo sapiens
ctttggacacctgagcgaggaagatgttgagagtgttgcctgatagcatcagacttggaagcagctgctgttggccttagtggtgggagagcacccgatgcttgccctgagtggggccaggtctggttgctcagcacaggataccaagctggcctttcctgctaccgctttttttttttgaaacagagtctcattctgtcrccctgtctaaagtccagtggcatgatctcggctcactgcatcctctgcctcccaggttcaagcaattctcccacctcagcctcccaggtagctgaaattataggtgcctgccaccatgcccacccagctaatttttttgtattttcagtagagacgaggtttcactaagttgacaaagctggtctcgaactcctaacctc

SEQ ID NO: 485 - DNA, 401 bp, Homo sapiens
accgcaagctccgcctcccgggttcatgccattctcctccctcagcctcccgagtagctgggactacaggcgcccaccaccacgcccggctaatttttgtattttttagtagagatggggtttcatcgtgttagccaggatggtcttgatctcctgacctcgtgattggccttccttggcctcccaaagtgctgggattayaggtgtgagccaccacgcccggcctctggaaaggtttttaatgatttttacaaatattaaaaattacattaatatgtcagattaaaaaaatatatataggccagctgcagtggctcacgcctgtaatcacagcactttgggaggcggaggcaggcggatcacgaggtcaagagattgagaccatcctggccaacatgatg

SEQ ID NO: 486 - DNA, 401 bp, Homo sapiens
aaccttctgagtagctgggattacaggcatctgccaccacatttggctaattgttatatttttagtagagacggggtttcaccatgttgaccacgctggtctcaaactcgacctcaagtgatccacctgcctcagcctcccaaagtgctgggaatacaggcatgagccacagtgcccagcctatatattagatttttaagyggaaaggtgtttgttaagaatcttagatcacttgagcccaggaaattgaggttgcatgccactgcacgccagcctgggtgacacagagacactctgtctcaaaaaataaaagaaaaatccaaacagttgtgttctaagtaggatatgtagtaatcatgacctttagcattgtactgcagcagtctatcatctaataagtg

SEQ ID NO: 487 - DNA, 401 bp, Homo sapiens
aattgaaagaaggggtccaggtgtggtggctcatgcctgtaatcccagcactttgggaggccaaggcgggtggatcacctgaggtcaaggagttcgagaccagcctggacaatgtggtgaaaccctatctctactaaaaatacaaaatttttagtatggcatggtggtgggcgcctgtaatcccagctacttgggaggctragacaggagaattgctcgaacctggagggcagaggttgcagtgagccagataacgccactgcactccagcctggacaacagagagagattccatttcaaaaaaaaaaaaagccataggagcttaggagtttgagaccagcttgggcaacatagcaagaccccatttcaactttttaaaacaaaaaaataaaattttaaaa

SEQ ID NO: 488 - DNA, 401 bp, Homo sapiens
tttttcagctattttagcaaggtaggtttacatgaagagtatgctatttggaaatatttattcgtccattcattactcatttcacagtaggggaaacattcctgtttctcccccgtggtggtgataagtggaaatagcttctttgaacgtgtgcagggtttcactgaccttggcccacccctgtgacgtggggcctcagtmagcgaagtaccaactctccgggagtctatggactcagcctgcagagcctggaggagccacttctggtggacatggggtgcagaaggaaacatcagactcatccaccctgcccgtcacctgcaggttttctaaagtgtggaggagtgtgaagctacagcaaggctggcccttgtagagcagttttcattgtgtgctctctg

SEQ ID NO: 489 - DNA, 401 bp, Homo sapiens
ccccccaggctggagtgcagtggcgcgatcttggctcactgcaagctccgcctcccgggttcacgccattctcccgcctcagcctccagagcagctgggactacaggcgcccgccaccacacctggctaattttttgtatttttagtagagatggtgtttcaccgtgtcagccgggatggtctcgatctcctgacctcgtkatctgcccgccttggcctcccaaagtgctgagattacaggcctgagccaccgtgcccggcctgaagtggacactcttgatgaacaccgttgctccagggtgccctccaggtagactgaggctgtgctgggcccacaacacagttcagttttttcctctgccccatcagtttttcctccttcccttcctaggtgtggatct

SEQ ID NO: 490 - DNA, 401 bp, Homo sapiens
actgtcagctctcagtttcccagtggctccaggtgtttgagatgttaagcgagctttttaaaatgctgcttaaatccacatgccttttccctgacataagtacagctaagacaaatactcactttatacacatcctgcgtaggcaaggtgaagaaccacagctataggttttgtttgtttatttgtttgttttgagacggmatctcgctttgtcccccaggctagagtacagtggcgcgatctcagctcactgcaagctctgcctcccgggttcatgccattctcctgcctcagcctcccgagtagctgggactacaggcgcccactaccatgcctggctaatttttttgtattttcagtagagacggggtttcactgtgttagccaggatggtcttgatc

SEQ ID NO: 491 - DNA, 401 bp, Homo sapiens
gtggctcgtgtctgtaatcccatcactttcggaggccgaggcagatggatcacctgaggtcaggagttcaagaccagcctggccaacatggtaagaccctgtctctaataaaaatacaaaaattagctgggagtggtggcgggtgcctgtaatcccagctactcaggaggctgaggcaggagaatcacttgaacccgggargtggaggttgtggtgagccgagatcccgccattgcattctagcctgggcaataagagcaaaactccatctcaaaaaacaaaacagaacaggcttcagtgccccaaggcacaagggaatggaggcaaggacaggaggatgttgcccacaggtgacattataaaggaattttatgagaacaagcttagggagaacaaacaga

SEQ ID NO: 492 - DNA, 401 bp, Homo sapiens
agtgaccctcctgcctcagcctccccagtagctgggactacaccacacccaactaattttgtagttttctgtagagatgggatctcgctatattgtcccagcacacaccaccacacccaactaattttgtagttttctgtagggatgggatcttgctatattgtcctggctggtcttgaattcctggtttcaagtgatccycctgccttggcctcccaaaatgctgggattacaagtatgagccaccatgcctggccaaaaaataccattttaaactgtatatatcactgctttatagcatattcacagagttgtctaaccatcatcacaaccaatgtttgaactgttcatcatttcaaaaagtcactccataacctttagctatcaccccagtcccctat

SEQ ID NO: 493 - DNA, 401 bp, Homo sapiens
cacccttctactttccgtctatggatttgactactttagataccaaataagtgaaatcatacagtgtttgtctttctgtgactgacttatttcatttagtgcaatgcctttaaggttcatcaatgtcgtagcatgtgacagtattttcttattttttaaggctgaataatgttacaattttaataattatatatcattatwataactgaataatatttacaacaatacaattgcattttatgtataagtacattttaaaatccattaatgacatttgggttgctttcacctcttggctattgtgaataatgctgcagcaagcatgagtgtgcaaatatttctttgggatcctgctttcaatacttttgggtatatacccaaaagggggattgctggaatat

SEQ ID NO: 494 - DNA, 401 bp, Homo sapiens
tctcgctctgtcgcccaggccggactgcggactgcagtggcgcaatctcggctcactgcaagctccgctttccgggttcacgccgttctcctgcctcagcctcccgagtagctgggactacaggcgcccgccaccacgcccggctaattttttgtatttttagtagagacggggtttcaccttgttagccaggatggtctygatctcctgacctcatgatccacccgcctcggcctcccaaagtgtcgagttaatttttgtatatgaggttacaggcatgaaccaccattccccgctatcaactttattctttttgcatgtggatatagttttcccaacaccatttgctgaagagactgtccttctcccattgtgagatgttggtgcccttgctaaagatc

SEQ ID NO: 495 - DNA, 401 bp, Homo sapiens
ctctcaggttcaaacaattatcttgcctcagcctcccgagtagctgggattacaggtgcctgccactatgcccagctaatttttgtattttttagtagagacagggtttcatcatgttaaccaggctggtctcgaactcgtgacctcaagacatccgcccgcctcagcctcccaaagtcctgggattacaggtgtgagccmctgcatctggccattgaacatctttttatgtgcttcttggccagtagaggacccttaaaaaacttttaattttgacctagtttcacatgtacagaaaagttacagaatagttaaaacaaaaaactatatacccttcacccagattttccagcgttaacactttgctccatttcagctattatttcctctctctctcactc

SEQ ID NO: 496 - DNA, 401 bp, Homo sapiens
ctgattaacgtggtgaaaccccgtctctactaaaaatacaaaaaaaattagccgggcgtggtggcaggcgcctgtagtcccagctactcaggaggctgaggcaggagaatggcgtgaacccgggaggcagagcttgcagtgagccgagatggcgccactgcactccagcctgggtccagcctgggcaacaagagtaaaacyctgtctcaaaaaaaaaaaaaaaaagataaataataaaaaataaaatataaataaaaaagcaaactgcagactctcagcacaaatgatttcatccacctctggcactaagagacttcaacaaacactgttgtttctaacagttcatggccacatccttaggatgatgacttcagcccccttaagtttctgcctaataaaat

SEQ ID NO: 497 - DNA, 401 bp, Homo sapiens
ctggtgcaaaatttttctttaacaatagcaagtgttgaaattggatagtgtaagttttttttttagctttgttctttttcagaatatttaggctatcctaggtcattcgcttttctatataaattttagcaaaagcttatcaatttccacaaaaacctgctggactattttttttgtttttgtttttgagaccaagtctcmctctgtcatccaggctggagtgtggtggcacaatcttggctcactgcaacctccgcctcccaagttcaagcgattctcctgcctcagcctccctagtagctgggattacaggcagatgccaccatgcccagataatttttgtatttttagtagagtcactgtttcaccatgttggccaggctggtcttgaacacctgaac

SEQ ID NO: 498 - DNA, 401 bp, Homo sapiens
ttttaaattggcagaaatttatcaataaattgggggaggactgtcatcttaacaatagagtcttttaatctatgaacatgggattatttaaatcagggattagcaaattttctgcaaagaaccagatagtaaaaatatttgtggtattgtgggctatattcaactttgctttgtagtgtaaaagcagtgttggcccggcgyggtggctcatgcttgtaatcccagcactttgggaggctgaggcgggtggactacctaaggtcaggagttcaagaccagctggctaacatggagaaaccccgttactactaaaaatacaaaaaaaatagccaggcatagtggcgcacgcctgtaattccaactattcgggaggctgagacaggagaatcgcttgaacctgg

SEQ ID NO: 499 - DNA, 401 bp, Homo sapiens
catcttaacaatagagtcttttaatctatgaacatgggattatttaaatcagggattagcaaattttctgcaaagaaccagatagtaaaaatatttgtggtattgtgggctatattcaactttgctttgtagtgtaaaagcagtgttggcccggcgcggtggctcatgcttgtaatcccagcactttgggaggctgaggcrggtggactacctaaggtcaggagttcaagaccagctggctaacatggagaaaccccgttactactaaaaatacaaaaaaaatagccaggcatagtggcgcacgcctgtaattccaactattcgggaggctgagacaggagaatcgcttgaacctgggaggtggatgttgcagtgagctgagatcacaccattgcactcca

SEQ ID NO: 500 - DNA, 401 bp, Homo sapiens
acaattgatttttacatatcggccttatatacttgaaccttgtttgctaaactcatttattaattccagtaaaattttttgatagattacttgggattttctgcatacagatcatgtggtctacaaataaacacagttttacttttttcttttcattctagatgtctttaatttacctttctctctctttctccctctacmccctttttaagttgccctggctatgaccagtagtacaatgttgaatggaagtggtaagagtagacattcttgcctgtttcctaatcttgagtgggaaacattctgtctttcatcattaagtacattaactgtaggttgtctgtagttgccctctatcagattgaagaagtttacttttattcctagtttgttgagagttt

SEQ ID NO: 501 - DNA, 401 bp, Homo sapiens
tgtttagctttgatattggggtaacactggcctcattaaaggagttggaacatgatctatccttttttattttctgaaagatttttgtgaaagacataatatttcttctcgtcttattgtgtcgtgtcgtgtcctgtcctgtctttttgaggtggagttttgctcttgttgcccaggcgggagtgcaatggcatgatctckgcccattgcaacctccacctcccaggttcaggtgatgctcctgcctcccaagtagctgggattacaggtatgcaccaccagacctggctaattttttttttttttttttttttgagacaaagtcttgctctgtcacccaggcgggagtgcagtaacgtgatcttggctcactgcaagctccgcctcccgggttcacgcca

SEQ ID NO: 502 - DNA, 401 bp, Homo sapiens
gcagccctggctgtaaagagagcaggggcttggctgggcgtggtggctcacgcctgtaatcccagcactttgggaggccgaggtggcagatcatttgaggtcaggagtttgtgaccagcctggccaacatggtgaaaccccatctctactaaaaaaaaaatacagaaattagctgggcatggtggcatgtacctgtaatcmcagctacgcaggaggctgaggcaggagaatcacttgaacccaggaggcggaggttgcagtgagctgagatagcgccactgcactccagcccgggcgacagcgcgagactccatctcaaaaaaaaaaaaaaaaaaaaaaaaaagagagtactggcttttgggtcattaggactattatataactgaattgtgtcaggcctc

SEQ ID NO: 503 - DNA, 401 bp, Homo sapiens
gcggaggttgcagtgagctgagatagcgccactgcactccagcccgggcgacagcgcgagactccatctcaaaaaaaaaaaaaaaaaaaaaaaaaagagagtactggcttttgggtcattaggactattatataactgaattgtgtcaggcctctgagctgaagctcagctattataacccctgtgacctgcacatatatrtccagatggcctgcaggaaccaagaagtctggagcagccaaaaaacccacaaagtaaaacagccagttcctgccttaactgagtaaccaaaattacaacattttaccattgtgacttgtccctgccctaccttagctgatcaaccaactttgtgacattcttcttctggacaatgagtcttatgatctccccactatgta

SEQ ID NO: 504 - DNA, 401 bp, Homo sapiens
gtgcccccactcagcatgttggatcctggcccctccagacctcaacatcatccagaaacagaaggaatagagcctcgcacagtagggtcccccagactgacttctggcagctgaactgaagggtcagtggttgagccatactggttgttcaacactttgaataccacatccagaaagaaaatacaagctccctgtcacggkagcctccttgtgactgtgttacattcaacacaagacaaacatcagcttcacaaaagggagcagtctctgggacaatccattgaaagcgtgagaggcaggtagttcctgggttctgtgcatagttgctttaagctgtaaacacacaatccttgaccaaataccctttaatccacttgacaaacaagaagcttccgggcctc

SEQ ID NO: 505 - DNA, 401 bp, Homo sapiens
aaatcaagtttagcctaaagccttctccttacatattttaaattcaccctaatggtttctctgtacatagtgaactgtaagctaactgcacgtgtaaacaggctgtcacctactctcgtaccaagtagccgagtctcagttaatcacagcagccagacttcaaccactcacaggcggccagctgttgaaactggactcaartaagccaagcaccatgccatcaccaatccgtctgtttctgtacctcacttccagtttctgtacatcactttcctttttctgtccataaatctttgactatgaggcagtgcaggagtctctctgaacttatttttgcctgggggctgcctgattcacacaaatggttctttgctcaatcaaactctgttaaatttaatttg

SEQ ID NO: 506 - DNA, 401 bp, Homo sapiens
aatcaagtttagcctaaagccttctccttacatattttaaattcaccctaatggtttctctgtacatagtgaactgtaagctaactgcacgtgtaaacaggctgtcacctactctcgtaccaagtagccgagtctcagttaatcacagcagccagacttcaaccactcacaggcggccagctgttgaaactggactcaaayaagccaagcaccatgccatcaccaatccgtctgtttctgtacctcacttccagtttctgtacatcactttcctttttctgtccataaatctttgactatgaggcagtgcaggagtctctctgaacttatttttgcctgggggctgcctgattcacacaaatggttctttgctcaatcaaactctgttaaatttaatttgt

SEQ ID NO: 507 - DNA, 401 bp, Homo sapiens
tgcctgattcacacaaatggttctttgctcaatcaaactctgttaaatttaatttgtctaacgttcttttaacactgggaatgatggcagggcacggtggcccatgcttgtaatcccagcactttgggagaccaaggcaggcagatcacttgaggtcaggcgttcaagaccaacctgggcaatggtgaaaccctgtctctrctaaaaatacaaaaattagccaggcatggtggtgcatgcctatagccccagctactgggaaggctgaagcacaagaatcgcttgtgcccgggaggtggaggttgcagtgagctgagattgcagcactgcactccatcctgggcgacagagcaagactctgtctcagaaaaaaacaaaaaacaaaacaaaacaaaaaaccc

SEQ ID NO: 508 - DNA, 401 bp, Homo sapiens
gacagagcgagactccgtctcaaaaaaaaaaaaaaaaaaaaaaaaaaaggtctaatcttgccaattagcaaagaaaaataaattaagacaaagcatttcccacatatcatagtaacaggtgataatctaagagaattataattttcttttacccagaaattctacctttgtgattttttttttttttgagatggagtttcrctcttgttgcccaggttggagtgcaatggcgtgatctcagctcacagcaaccttcgtctcccggtttcaagtgattctcctgcctcaccctccggagtagctgggattacaggcatgctccaccacgcccggctaattttgtatttttagtagagacggggtttctccatgttggtcaggctggtctcgaactcgtgacc

SEQ ID NO: 509 - DNA, 401 bp, Homo sapiens
gtgcaatggcgtgatctcagctcacagcaaccttcgtctcccggtttcaagtgattctcctgcctcaccctccggagtagctgggattacaggcatgctccaccacgcccggctaattttgtatttttagtagagacggggtttctccatgttggtcaggctggtctcgaactcgtgacctcacgtgatccacccacctcrtccttccaaagtgctgggattacaggtgcgagccacctcacccggcctctatgaatttatcttaaagaaattagtataagtgtgggaaatttttatctgaaggacctggatttcaacagcgtttcttttttttttttttgagacagagtctcgctctctcgcccaggctggagtgcagtggcgcgatctccgctcactgc

SEQ ID NO: 510 - DNA, 401 bp, Homo sapiens
ccgagctgtatgtagttcttgctctcctgtgccctgcaggatggcttgacccatttctgttctcatgtcagaccctaagggcagggtcatctgggccacagacccaggcccggtgggcctggtgggctgacctgtgggctattcttatgccttcaggaaacccacctttaccacctggctgttgtgactggcatcgctgasgctgtttctttgtgtataaaatcgaataaggacacatcacgtgctagcaatccaccagcatagggcgaaggctcagcaaagagaagttctctcctcctctgctcttttaggtcagcagcaaatgcagatcggggtggggacaaggtaaacacataacttgggtggagatgtgattgattaataactcataaatcatctga

SEQ ID NO: 511 - DNA, 401 bp, Homo sapiens
cgagctgtatgtagttcttgctctcctgtgccctgcaggatggcttgacccatttctgttctcatgtcagaccctaagggcagggtcatctgggccacagacccaggcccggtgggcctggtgggctgacctgtgggctattcttatgccttcaggaaacccacctttaccacctggctgttgtgactggcatcgctgagkctgtttctttgtgtataaaatcgaataaggacacatcacgtgctagcaatccaccagcatagggcgaaggctcagcaaagagaagttctctcctcctctgctcttttaggtcagcagcaaatgcagatcggggtggggacaaggtaaacacataacttgggtggagatgtgattgattaataactcataaatcatctgaa

SEQ ID NO: 512 - DNA, 401 bp, Homo sapiens
gctgtatgtagttcttgctctcctgtgccctgcaggatggcttgacccatttctgttctcatgtcagaccctaagggcagggtcatctgggccacagacccaggcccggtgggcctggtgggctgacctgtgggctattcttatgccttcaggaaacccacctttaccacctggctgttgtgactggcatcgctgaggctstttctttgtgtataaaatcgaataaggacacatcacgtgctagcaatccaccagcatagggcgaaggctcagcaaagagaagttctctcctcctctgctcttttaggtcagcagcaaatgcagatcggggtggggacaaggtaaacacataacttgggtggagatgtgattgattaataactcataaatcatctgaaacc

SEQ ID NO: 513 - DNA, 401 bp, Homo sapiens
gccacagacccaggcccggtgggcctggtgggctgacctgtgggctattcttatgccttcaggaaacccacctttaccacctggctgttgtgactggcatcgctgaggctgtttctttgtgtataaaatcgaataaggacacatcacgtgctagcaatccaccagcatagggcgaaggctcagcaaagagaagttctctcytcctctgctcttttaggtcagcagcaaatgcagatcggggtggggacaaggtaaacacataacttgggtggagatgtgattgattaataactcataaatcatctgaaaccatactttccttttgatagtcaaccctctgtaaacactcaatgtgttctcaccttgttatcattcccaatgatgtcgaatgcgtggttgcc

SEQ ID NO: 514 - DNA, 401 bp, Homo sapiens
gatcctgaggacccccaactctttatttatttatttatttattttattattgttatttgtagtagagatgagtttttactatgttgcccaggctggtctctaactcttgggcccaagtgatcctcccaccttggcttcccaaagtgctgggattacaggtgtgagaaattatgcctggccagacccccaactcttgatcaygggataacccttccaggagcagtaatttacctaaagagatacaacttttttttttttttttttttttttttttctgagacggagtctctctgtcgcccaggctggagtgcagtggcgcaatctcggctcactgcaagctccacctcccgggttcacgccattctcctgcctcagcctcctgagtagctgggactacaggc

SEQ ID NO: 515 - DNA, 401 bp, Homo sapiens
cgtcactatggcgtcatggtgatggccacctacctggatgacgtaatcatgggccgtgaggttaaaccagacccccccaagaaggaaggagactgcggggagctttgggatttccgagcacaggatgatgtactgggggagaagaggaactagtgaagatgagagattcctgctctgttcagtcccttccgtggctccccrgtgccatagggtaatttccctattgagcacctgggtactcagcgtcctgtgtgcagaggccctgccgcctagacttacgcagtcccaagcaaacctgcatgatcgtctccctccacccggccaccatccgcagagtcgcagtgtagacacgtggaaagtgtcctcaattcctccaccaccctccagactatctgtcacga

SEQ ID NO: 516 - DNA, 401 bp, Homo sapiens
ttgtatttttgtagcgacggggtttcgccttgttggctaggatggtctcaatctcttgaccttgtgatctgccctccttggccttccaaagtgctgggattacaggtgtgagccaccgtggccaacgggtatatctgcttatgggaatggggtttctttgtggactgaccaaaatgttctaaaattggttatggtggtggytgcacaacccgtgaacttgctaaaaaccattaaattgtacttgaaagtgctgaattgtttggtgtgtgaaccaaatctcaataaagctattaccaaaaaaattagagaatattgggttacttacaaatgagaaccttggtccacagctaccttcagacaacaaaactgccagtgaccgagtgcttctgtatgccaggcac

SEQ ID NO: 517 - DNA, 401 bp, Homo sapiens
ccagcactttgggaggccgaggtggacggatcacttgaggtgaggagtttgagaccagtctggcagacatggtgaaactctactaaaaatacacaactagctggacatggtgggcgcctgtaatcccagctacctgcgaggctgaggcaggagaatcacttgagtccaggaggcggaggttgcagtgagcaagatcctttsccaaaaaaatatataaataaaaataaagctgtgagattgggcaagattggatgaggtcattgaggaaatgagtgtctcactcatcgagaaaactggtgtctggcctgcagcgagcacgtggtgtcattttgttgctgaatctctgttaagtgcttgctaccgaagggtcgtgagaggggcagaggacagtgtttgccaag

SEQ ID NO: 518 - DNA, 401 bp, Homo sapiens
caggtgtgagtcactgtgcacggccagttgtttttatcttgtaataaaaaagaagggcctccctgtagttgtgctgtggagaatggatcctgggggatgaggctggagatgggaaccaggggggaggctgaaatggacacagggaacggtacaggggcaggagtgccccaggctgcaggccccaaggtatttcgaggctgkggagacacaccttggaggaccctgagaggcatggccgctgtcttaccctgtgaccccacaggcccagcgcagcctgtgcctggggggcccccaccatcccgcggctccgtgcctgtgctccgcgccttcggggtcaccgatgaggggttctctgtctgctgccacatccacggcttcgctccctacttctacaccccagc

SEQ ID NO: 519 - DNA, 401 bp, Homo sapiens
acggcctctgcctcacttctccggcctctatccccaccctcgggcagcccctgtccactgacccccagccccctccaggttcatggtggacacggacatcgtcggctgcaactggctggagctcccagctgggaaatacgccctgaggctgaaggagaaggtgcagggcttcccagggcagggctgggtggggagctggtwccctgctgccaccgctgacccacccatgcccacaggctacgcagtgccagctggaggcggacgtgctgtggtctgacgtggtcagtcacccaccggaagggccatggcagcgcattgcgcccttgcgcgtgctcagcttcgatatcgagtgcgccggccgcaaaggtctgtccccgggcccgggctcctgcccgcctcat

SEQ ID NO: 520 - DNA, 401 bp, Homo sapiens
tccggtggagggaatggcaagcatgaaggtgccggggcaggagcaccccagcccatgtggccagatggagtgagcacagagggtgtggagatggcctggaggtgagagcagagcaggagccagggtgagccacgtagggccggcaggcagcggggacagccccggggagatggcaggtgcagcctccctgctgtgttgggmgtgaggggcaggagtcaggcccctgcatcctcctgcctcgcaggcatcttccctgagcctgagcgggaccctgtcatccagatctgctcgctgggcctgcgctggggggagccggagcccttcctacgcctggcgctcaccctgcggccctgtgcccccatcctgggtgccaaggtgcagagctacgagaaggaggagga

SEQ ID NO: 521 - DNA, 401 bp, Homo sapiens
ggcgctggggtgtagaagtgattctcctgcctcagcctcccaagtagctgggattataggcacccgcttccatgcccgactaattttgtatttttagtagacagggtttctccatgttggccaggctggtctcgaactcccaatctcaggtgatctgcccacttcggcctcccaaagtgctgggattacaggcgtgagccwccgcgcctggcctatttttgtttttgtttttgttttttttagcagagactaggtttcatcatgttggccaggctggtctccatcctcacctcaggtgatctgcccacctcagcctctcaaagtgctgggattacaggtgtgagccagtgcacctagcccacatgtcctctttgtgtagtgacaccgtcgtgaggtgaggg

SEQ ID NO: 522 - DNA, 401 bp, Homo sapiens
ttaggacacaagtggcgctacgagctttgcagccggataaagcaggaggtgcggactgtcggggagtgatggggctgggcgggactcacccctctcaataggcgacgctcacacagaggccaagggacgtggggagggaagccattcaccgtccccacggcagcgtcccggtctgcctacccacggcagctctgctgcttkaacagaaaaacagattctttgaaaccacagagggcctgagtttccgagattgccaagctggggtcagtgccccagctcctggcatatgagggcctcggaagggtcttcctgtccagccttctcccctgggacttgagtcttggcccattccaggccagtagttgtgctgctctgcttggttacctctagccacggggagc

SEQ ID NO: 523 - DNA, 401 bp, Homo sapiens
aagctttggggtccagggtttttcttgggtgtcagtcctataggcatggagcaccctgtgactggccttagtgactccatcatccccaggcccctcagcaacccccagaggtcaaactgatactgcatggcccagggccctaccataaaccatgttagtgtcaactacctggtgtggttcaaagtcccaggtacacaaacmccttttttttttttttttttgagacagagtctcactctctcacccaggctggagcgcagtggcacaatcgtggctcactgcagcctcaacctccgggactcaggtgagcctcccacgttagcctcccaagtggctgggactgcgggcacgtgccacctatttttgttattttttgtagagatagggtctcgccatgttgc

SEQ ID NO: 524 - DNA, 401 bp, Homo sapiens
caaaaaatacaaccaaacaaaactagccaggcattgtggtagctcatgcctgtagttgcagctactcaggaggctgagacgggaagatggcttgaacccagcagtttgagttacagtaagctgtgatggcatcactgcactccattctgggtgacagagttgagaccctgtctcttttagtttttgtttgtttgttttgaratggagtcttgctctgtcacccaggctggagtgcaatgccacaatcttggctcactgcgacctctccctcccgggttcaagtgattctcctgcctcagcctcctgagtagctgggattacaggcacccgccaccatgcctggctaatttttgcatttttagtagagacggggtttcaccatgttggccaggctggtcttg

SEQ ID NO: 525 - DNA, 401 bp, Homo sapiens
gccctgtcagggacacccttgttatatctgggatcctccaatcacatctgagacctcctaggctctccatctgatatgccctttcagggaccccacaaagactgagttctcatggggatcctacccttcctagtgccactccctatggccatgctgaagaccactctggccacgcgactgattttgggtgatcatggcagmtccccacccatgtcatttctaaccagaagtctcaaggtcgtcacccccctgccccccaaccgaggccccggtcgctggtggtggtctctttagtgcactgtagcacttggtggtggaggtgtgagggatccacattaacagcaggccatcagctgggcaatggctcacacctgtaatcccagcactttgggaggcgaggc

SEQ ID NO: 526 - DNA, 401 bp, Homo sapiens
gcatgctcaactatgactgtcccctaccttacagcggccaaaaaggcccccaaaggcaaagatgcccccaaaggagcccccaaggaggctccccctaaggaggctcctgcagaggcccccaaaggtgaggaggtgctccctcgggctcaaccgacctggcttctcatctccatcctcctcgctacgcctctccaggccttyccccagcctctatccttgaatctgtccccgcacagccccagggctgacccacgcctcccgtgctcccagccctcccggggctccctggcacccgcaggcagagcccagcccctcggctggcatcggctccggtcagctggctgattgatcaccaggtccggccccggtctctctaaacctctcccgccccaaaggcacat

SEQ ID NO: 527 - DNA, 401 bp, Homo sapiens
tacgcctctccaggcctttccccagcctctatccttgaatctgtccccgcacagccccagggctgacccacgcctcccgtgctcccagccctcccggggctccctggcacccgcaggcagagcccagcccctcggctggcatcggctccggtcagctggctgattgatcaccaggtccggccccggtctctctaaacctcycccgccccaaaggcacattcagtgccctccagtccacagatgaggaaaccgaggcccaaaggggtgagggagcctggccaaggtcacacagccacgagggtgacaggtggcctttcccaggtcaggaaaagccatttaacccccatgatgtttggtttctgagtagaggggccctcactgtctctaagtcctttccccaa

SEQ ID NO: 528 - DNA, 401 bp, Homo sapiens
ccctggcacccgcaggcagagcccagcccctcggctggcatcggctccggtcagctggctgattgatcaccaggtccggccccggtctctctaaacctctcccgccccaaaggcacattcagtgccctccagtccacagatgaggaaaccgaggcccaaaggggtgagggagcctggccaaggtcacacagccacgagggygacaggtggcctttcccaggtcaggaaaagccatttaacccccatgatgtttggtttctgagtagaggggccctcactgtctctaagtcctttccccaaagcggcccactgtcccctccccagcctatctcagacctcctcatcctaatcctgtcccctgaacagaagccccacccgaggaccagtccccgactgcagag

SEQ ID NO: 529 - DNA, 401 bp, Homo sapiens
gcacattcagtgccctccagtccacagatgaggaaaccgaggcccaaaggggtgagggagcctggccaaggtcacacagccacgagggtgacaggtggcctttcccaggtcaggaaaagccatttaacccccatgatgtttggtttctgagtagaggggccctcactgtctctaagtcctttccccaaagcggcccactgycccctccccagcctatctcagacctcctcatcctaatcctgtcccctgaacagaagccccacccgaggaccagtccccgactgcagaggagcccaccggcgttttcctgaagaagccggactccgtctcagtggagactggtgaggggaacccgggggaggaggggctgcggcccggactcctgggtctgagggaggagg

SEQ ID NO: 530 - DNA, 401 bp, Homo sapiens
ctcctgggtctgagggaggaggggccagggtctcgttctgtctccctctggagagccttatgttccttcctgggcctcctcccactctaccctgtcactcaccccatgtagggaaggacgcagtggtcgtggccaaggtgaacgggaaggagctcccagacaaaccgaccatcaagtggttcaaggggaagtggctggagmtgggcagcaagagtggcgcccgcttctccttcaaggagtcccacaactccgccagcaatgtgaggaccccgtgggccagagggctggtggaggggagtggagcttgtgcaggacatgctcctcagaagccctgttgcctgaggcatcaatgtgggcctggagccgggatggagagtgggccctctgaggatggaggttgt

SEQ ID NO: 531 - DNA, 401 bp, Homo sapiens
ggtgtacaggattaacaggaggttacacggatttgagggcacatatatgggcccctgagggtgtatgggctctgggggtctgcagcccagaatgtgtgagggcttaaatgtggggtgcacaggaaatgagattgtccaggcctggggagaaagacatggtggcccaggtgtggggggtataggctgctgagagagggtggwgcttggcagctggggaaatgagtatgagctggtctagaactagaggatgcccaggcctgagcaaggttgtgctggtccaagagatggagaaaagggggcctggaagaggttgtgcaggcctggagaggggctctcagtctaagatcagccctggcctctaactcaccttcccttctctcatcacctctccccagcacccc

SEQ ID NO: 532 - DNA, 401 bp, Homo sapiens
aggaggttacacggatttgagggcacatatatgggcccctgagggtgtatgggctctgggggtctgcagcccagaatgtgtgagggcttaaatgtggggtgcacaggaaatgagattgtccaggcctggggagaaagacatggtggcccaggtgtggggggtataggctgctgagagagggtggagcttggcagctggggwaatgagtatgagctggtctagaactagaggatgcccaggcctgagcaaggttgtgctggtccaagagatggagaaaagggggcctggaagaggttgtgcaggcctggagaggggctctcagtctaagatcagccctggcctctaactcaccttcccttctctcatcacctctccccagcaccccgtcaggatgcctctgg

SEQ ID NO: 533 - DNA, 401 bp, Homo sapiens
gcttgggtgagtcacagagccaatgagtgaagaaggcagagggacatcctcccttcctcactccctgatggcccctgtccgttcccccacccgccagggaggtggtggaggaggagaagaagaagaaaaagaaagatgacgatgacctaggcatccccccggagatttgggagctcctgaaaggggcaaagaagagcgagyacgagaaaatcgccttccagtatggcatcaccgacctccggggcatgctgaagcggctgaaaaaggctaaggtcgaggtcaagaagagtgcaggtcagccctggtctggggggagctgggccctgcacacaagggacccctagccttgtgggtgtctaatgattggaccctggacctatcttttcgaggtcccaatgagg

SEQ ID NO: 534 - DNA, 401 bp, Homo sapiens
gagagcccagggcctggggagggagatgtggtgcctctggggatgaccaggggaatgaggaccacacgcctccatcccctttgcatgcctcaatcttcagcattcacaaagaagctggatccagcctaccaagtggacagaggcaacaagatcaagttgatggtagagatcagcgacccagacctgaccctcaagtggttmaagaacggccaggagatcaaaccaagcagcaagtatgtgtggggtgggcagtccctgcaccggggagatccccgtccagagagaacgcccggagacaaggcctatcactcttaaccagaaagggcatattcacccctatatgaagagtacaggccgggcacagtggctcacgcctgtaatcctagcactttgggaggccg

SEQ ID NO: 535 - DNA, 401 bp, Homo sapiens
ttgggaggctgaggcaggagaatggcatgaacctgggaggcggagcttgcagtgagccgagatcgcgccactgcactccaacctgggagacacagcgagactccgtctcaaaaaaaaaaaaaaaaaaaaaaaaatgtcaacatggtacctgccttcatggcatttataatctgctaatttggtgatagtggtggtggtggygtgtgtgtatgtgtgtttgtgtaggagaaagagataggcattagtcaagtcatcacatacatacatataaaagttccattttgagactggcttgaaaggagcagtaatggatgttgtaatagcttcctaggaggaagggactcaggaggtcagggaagtcttcctggaggagatgacaattgagctgagatctgaatgac

SEQ ID NO: 536 - DNA, 401 bp, Homo sapiens
tcccaagcaaggtgagcaccacgggctgcgctgggagcgggtctgagggaggagggaccgggctgagggaggaggggaatggccgagggaggataggcagggcgaggaaggatgggcggggtgcgggaggatgagtggggagaggaaggagggggttggggtgctgaggaagggagggaggaggggcggggagctgacagrcggggtgggggctatcggccgagaggagggactgaggtcctgagggaagagggattggggacctggatccctgggtctgagggaggaggggctgggggcctagactgtagggtgtgagggaggaggggttggggacctggatccctgggtatgagggaggaggggctgggggcctagactgtagggtctgagggaggagg

SEQ ID NO: 537 - DNA, 401 bp, Homo sapiens
acggcatttggagggcgaggagggcagtggagcgagtgaggactctgtgtgttgatggggaatcatgggaggattctgagcaggggaatggcagaggcaggcctgggtttgcttggggacacgatggggtgcaaggccatctggtggctgcgttcaggtattcacgaccaccgagggcaggacccgcatcgagaagcgggyggactgcagcagctttgtgattgagagtgcgcagcgggaagacgagggccgctacaccatcaaggtcaccaaccccgtcggcgaggacgtggcttccatcttcctgcaagttgtaggtgagcagagaaagccgaggtggctgggccaaggggtgactccctgaggcctcgaggggccacctgactctcctgccccctgca

SEQ ID NO: 538 - DNA, 401 bp, Homo sapiens
gtccacctctggcccatcttttgcccgccatcaatctctgaccctaccccctggccttctgaccaatcattctacctctgactcctgccccttcctgctgacccctgacccctcattccccccatatcctctttttctggatgcttgcagcacccacgagtgaacccctgcacctgatagtggaggatgtgacagacaccmccaccacactcaagtggaggcctccgaacaggatcggggcaggtggcatcgatgggtacctggtggagtactgcctggaaggctgtgagtgaccctggcagggctggtgggggtggtggctagcacaggtgggtatccagtccagggagctgaaaataggaccatgtgggacagagatgggtgcagtggcctgcttgtgt

SEQ ID NO: 539 - DNA, 401 bp, Homo sapiens
tggaggcctccgaacaggatcggggcaggtggcatcgatgggtacctggtggagtactgcctggaaggctgtgagtgaccctggcagggctggtgggggtggtggctagcacaggtgggtatccagtccagggagctgaaaataggaccatgtgggacagagatgggtgcagtggcctgcttgtgttttgtgtctgtactsttataagtctctgggatgggacccccctgtgactagtgtgttttctctcccttcttcctttctacatccatcctccatacagcagtgactttctgtcattccatctatccttccatccccccatttatcttttatcttttacctttatgccttctttctttcttctactcatttatccatctaaccaatctttccttcat

SEQ ID NO: 540 - DNA, 401 bp, Homo sapiens
aagacagttctcatcttgacagggtacagtgacgatgtactcgagaatgcatggaaaggagggatggttgaaccctcatcagcttcctccctcctcctctcctccgtgctctttaccccattcctccctggccggctgctcccaccctgacgtttcagaacccagcttgttagagttcactctgttgattgctgctctgarccaacagccatgagttccccagcccatggccataaacccaagacagaagggcatgtaacgaaaagcaggcaatctgaagagctctgaccctgccctcagcccgccttcccagacacccacctggctgcgccctcagacagagccaagatccctgcccctctgtgccagccttgcaggagagcagagcttgagagcgtgct

SEQ ID NO: 541 - DNA, 401 bp, Homo sapiens
aaaccccatctctattaaaaatacaaaaattagctgggcgtggtggtgcgagcctgtagtcccagctactcaggaggctgaggcaggagaattacttgaacctgggaggcggaggttgcagtgagccaagatcttgccattgccctgcagcctgggcgacagggcgagactccacctaaaaaaaaaaaaaaaagaattcgycccactgcactccagcctgggcgacagggcgagactccgtttaaaaaaaaaaaaaattaatgagcgaacagaatggccacccttagaacctggccatgatgaatgcttaactaactccagctgctgctgctgcttctactgggaacttactcagtgctcactcgctcgctgcgtggggcccaagtccccgggagaaacgg

SEQ ID NO: 542 - DNA, 401 bp, Homo sapiens
tggcccagccggtcaccatcagggagattgcgggtgcgtggcccctgaccctgcgctccgaaagaccaagctggcgtcgtcccgcctgccctttccgtgtcgtcgacaggacctccccagagagccttatcaccattggcccctggaatgcgccccggccccccgctgagccccctccctgtgtttgcgccctcagagccwcccaagatccggcttccccgccatctccgccagacctacatccgcaaagtgggcgagcagctcaaccttgtcgtccccttccaggtcaggggagcggggtcacgggccgggggtccgctctcgccgcaagccccctttttgcgggtggagccccgctgaccccaccccgccccgccctctccccgcagggaaagccccgg

SEQ ID NO: 543 - DNA, 401 bp, Homo sapiens
agaggaggtgagagatgagggatgggagaggaggtgaggagatgaggaggggagggggagaggaggtgaaaagatgaggtgggggagactagataaggagcaaggcaagtaccagggctaggagtggaggagagatgaagatgggggaacccttgagagggagattgggtgaagagaggaagtggggagacatgggggtgmggagaggaggtgaggagggatgagggtggcccaggataagaggaagtggagagactgggatgggggaggccataggcaggaatgggaatggggcataagagaggttagaatcaggagggagggggtgtggagaggggaggggagggaagcggctggacatcccaaaacctggactgacttgtcacacttccccatttcca

SEQ ID NO: 544 - DNA, 401 bp, Homo sapiens
gtagctaggattacaggcgtgcaccaccatgcccagctaaattttgtatttttagtagagacggcgttttgctgacctcaggtgatccgcctgcctcagcctcccaaagtgctgggattacaggcatgagccaccgctcctggcctcttgcctttttctttgtcccctgactgccaggtaacagcttgtctcggcagcttraggagaggaccattaagtctctcttgctctgtccaaggacaaatgccagtcccatggaaaaagaaacgttgggagcaggggcagcctagaccagcacggctccccagggtcttcctgtccagccattttccaactgctctttgtgaacagggacacttgcaacctagagctcatttagaccaggggttggcaaaccatca

SEQ ID NO: 545 - DNA, 401 bp, Homo sapiens
actaaaaatacaaaaactagccaggcatggtggcaggcgcctctaatcccaaccacttgggaagctgaggcaggataattgcttaaacccgggaggtggaggttacagtgagccaagatcacgccactgcactccagcctgggagacaaagcgagattctgtctcaaaaaaaaaaaaaaaagggaagggcactatggaagrgggaacagccagtgccagcgtcctgaggtgggtgcctgtctggtctgtttgaaggacagtgaggtagcctgtttggctggagcagagaagtaatgggggtcaaagagtgtgggaccctgtgggccactacaaacagtgagctctttgtcctgagagcaatgggaagaccccaccgttaccctgtgaagtgggattatcaa

SEQ ID NO: 546 - DNA, 401 bp, Homo sapiens
tcagatttttccaaaggagttgaaggacaaggaagaggaaggctgagtgagaagggcttaggaaacgtccccaaggcctccagtgtacctgggcccccgtaggcagatgggttgacagagtgagagcaaatatggataacacggatgaccaggggtggcgggagctcccaagagtgcctccctccatagcctgtccccatrctcccacccacctgcccctgcctctccctgccccgtccctcatctcggtcccacagctccagccccagcgaaagctacatcctcttcctcccatattccatttccagccgcctccgtctccagtctctccctcctgccagccactttcacccttggactctggccccagagagctctctctatccagagctgctctgccc

SEQ ID NO: 547 - DNA, 401 bp, Homo sapiens
agatttttccaaaggagttgaaggacaaggaagaggaaggctgagtgagaagggcttaggaaacgtccccaaggcctccagtgtacctgggcccccgtaggcagatgggttgacagagtgagagcaaatatggataacacggatgaccaggggtggcgggagctcccaagagtgcctccctccatagcctgtccccatgcycccacccacctgcccctgcctctccctgccccgtccctcatctcggtcccacagctccagccccagcgaaagctacatcctcttcctcccatattccatttccagccgcctccgtctccagtctctccctcctgccagccactttcacccttggactctggccccagagagctctctctatccagagctgctctgcccca

SEQ ID NO: 548 - DNA, 401 bp, Homo sapiens
aatatggataacacggatgaccaggggtggcgggagctcccaagagtgcctccctccatagcctgtccccatgctcccacccacctgcccctgcctctccctgccccgtccctcatctcggtcccacagctccagccccagcgaaagctacatcctcttcctcccatattccatttccagccgcctccgtctccagtctcwccctcctgccagccactttcacccttggactctggccccagagagctctctctatccagagctgctctgccccagccctgatcccagcccgctggggactccccaccgccccagcgcccctccgcctggtgttcacggtggtgtgtccaccccggttcttcccacatgatgtcccagccacaccgggctgttggcagtta

SEQ ID NO: 549 - DNA, 401 bp, Homo sapiens
tggataacacggatgaccaggggtggcgggagctcccaagagtgcctccctccatagcctgtccccatgctcccacccacctgcccctgcctctccctgccccgtccctcatctcggtcccacagctccagccccagcgaaagctacatcctcttcctcccatattccatttccagccgcctccgtctccagtctctcccwcctgccagccactttcacccttggactctggccccagagagctctctctatccagagctgctctgccccagccctgatcccagcccgctggggactccccaccgccccagcgcccctccgcctggtgttcacggtggtgtgtccaccccggttcttcccacatgatgtcccagccacaccgggctgttggcagttacact

SEQ ID NO: 550 - DNA, 401 bp, Homo sapiens
gttggcgacgggagcagctgcgtcgacacaggccgcggtgaggtggcctcttggaatagctactttagcgaggcagccacgttgtcacagcaaccacagagcatcagaggtgaacccaagcccacggggaggccggccccagagaagctgctgggagggagggagggtgcaggctaggggcaacgacagtggcagcgagtyaaagacacacccaggccacacccagaaggccactctaccgaagcagctgtgtcgctgtggccgcctctaggccttggcagcggccccaagagctagacgtggtgaaccgaagagggaggcagccgtactggacaaactcctgccaggcagctgcgcggacttggtgacgaaggcgaaaccgctcgtggcctgcttgttgg

SEQ ID NO: 551 - DNA, 401 bp, Homo sapiens
gctactttagcgaggcagccacgttgtcacagcaaccacagagcatcagaggtgaacccaagcccacggggaggccggccccagagaagctgctgggagggagggagggtgcaggctaggggcaacgacagtggcagcgagtcaaagacacacccaggccacacccagaaggccactctaccgaagcagctgtgtcgctgwggccgcctctaggccttggcagcggccccaagagctagacgtggtgaaccgaagagggaggcagccgtactggacaaactcctgccaggcagctgcgcggacttggtgacgaaggcgaaaccgctcgtggcctgcttgttggttcttaatggactgacctttattaactttaatatctcatctgcccggccctgaggcaa

SEQ ID NO: 552 - DNA, 401 bp, Homo sapiens
accaccatgcctggctaatttttctatttttatatagtagagatggggtttcaccatgttggccaggctggtctccaactcctgggctcaagtgatcctctcgtctctcaaagtgccgggattataggcatgagccacctcactttgcctgccacctttaaaatgtatccacaatccaaactcttttaactccctcctccsccacccttgcgtccagccacctgcctcagccctcagtccctacagtctgttccccacaagtcccccagaaggcgcaggtcaacaccttgggcagtttgggtccctctgctcagagccttcctgtggctctggctcaacaggaataaacacaaagccctcaccgtggcccacagacctgcagagccccttgctccctctct

SEQ ID NO: 553 - DNA, 401 bp, Homo sapiens
gtccaacagggcagagcaggaagcagtcaggaaagagtgaggatcttggctcaggtcacacagccaggattggagcccaggtcccatgttcacctacaacactgagtagccaggtggcctctctggtccagcagtatcactgccaggaatggatctcacagacaccctcacacctgtgcacagacacatatgcgcggatgwcccctgccacaccgcctgtaaccgagatgccaacaaaccccagtgtccctcagtgggtgacttgatttttgtcccccaaatagaagaataacagctgacatttgtggagcacttactcagagccagttgctattatatttacccagtttttcccccgacaaccttatggggtgtgctctcctcttatccctgtcatccag

SEQ ID NO: 554 - DNA, 401 bp, Homo sapiens
agtgagccaagatcatgccactacactccagcctgggcaacaagagtgaggctccgtctcaaaaaaaaaaaagagtccaattgtgcaaatatgtctgaaagagtgtggtggctgtgctcatggcaatacaaaacggatatgggtgtcgtgctgacgagggggtgggcgcagggagaagtggcgtatggcttgtggctactsctgtttttttttttttttgagatggagtctcgcactgtttcccgggctggagtgtaatggcgcgatcttggctcactgcaacctccacctcctcggttcaagtgattctcctgcctcagcctcccgagtagctgggattacaggcgcccgccaccatgcccggctaatttttgtattttttagtagagagggggtttcac

SEQ ID NO: 555 - DNA, 401 bp, Homo sapiens
gagccaagatcatgccactacactccagcctgggcaacaagagtgaggctccgtctcaaaaaaaaaaaagagtccaattgtgcaaatatgtctgaaagagtgtggtggctgtgctcatggcaatacaaaacggatatgggtgtcgtgctgacgagggggtgggcgcagggagaagtggcgtatggcttgtggctactgctstttttttttttttttgagatggagtctcgcactgtttcccgggctggagtgtaatggcgcgatcttggctcactgcaacctccacctcctcggttcaagtgattctcctgcctcagcctcccgagtagctgggattacaggcgcccgccaccatgcccggctaatttttgtattttttagtagagagggggtttcactat

SEQ ID NO: 556 - DNA, 401 bp, Homo sapiens
cagcctgggcgactgagcgagaccctgtctcagaaaaagaaaataaggcgtgggagggggctcttgaggaagtggagatgagttttgtctcctaaatatgcgtgagtatgcaagtttggaggttggagaaaaggaaagcatgaatcttgaatgtgcttaatagggactgaactcaggctgcatcttggctattgggctgtyaggtgataaggaggccatttgaatattagcatgaaaaagaaggcttgggagagggagagtgtcatttaactcatgagtatttgtggcgcaaggcttggaagtgaccttgggatagttgctttggcaatgaggttaggcatgtttgtagggtctggttttctgggggtgagggagtgcagagtatgaatattcatgaaatg

SEQ ID NO: 557 - DNA, 401 bp, Homo sapiens
gcactcatttgagatcggtgagtcaggcaacgtcctctcctagacacttaacattggtgccctgagaagtcaagacttggagtctgggtgcctcccgtcgtatgacatttactaacaaccatttactaacagatttgcctcaggttaggggaaagtgggacatgaaagaagagaaacactctttaaatgcccccttgcctrtgagacaaacctcagtttttaaaaggtcatcacagcagtaataatttcagagcggctttcctgatcagtcgccacgtgcccagcattagcggttccacgctttgcaagtattaactcagatgtcctcacagcagccatgtgaggtggtaggtattcgtgtcagcccctttttccagaagaggaaacgggctcagagtagg

SEQ ID NO: 558 - DNA, 401 bp, Homo sapiens
ggctcactgctctggaaccagcaggatggtaccttgtccctgtcacagcggcagctcagcgaggaggagcggggccgactccgggtgaggtggggccctcagggctgggtgtggatggggatggagggtttccagctgtctcttcagccaccccctgctggtccccagcagccttccctccaggcccaggtgggctcccascaccacctgccactctcagcctctgtcctcaatgactgcccctggaggaacggcattgcagttcgtcttagcgccgccaggcatggtgtcctgcatcacggcctcttcctgcactgctccctctgctggcagacccccttcccctgcaggttcacacagtcggctcctcttcttttccagatcagctgagctgtttcatg

SEQ ID NO: 559 - DNA, 401 bp, Homo sapiens
ctcactgctctggaaccagcaggatggtaccttgtccctgtcacagcggcagctcagcgaggaggagcggggccgactccgggtgaggtggggccctcagggctgggtgtggatggggatggagggtttccagctgtctcttcagccaccccctgctggtccccagcagccttccctccaggcccaggtgggctcccagcwccacctgccactctcagcctctgtcctcaatgactgcccctggaggaacggcattgcagttcgtcttagcgccgccaggcatggtgtcctgcatcacggcctcttcctgcactgctccctctgctggcagacccccttcccctgcaggttcacacagtcggctcctcttcttttccagatcagctgagctgtttcatgac

SEQ ID NO: 560 - DNA, 401 bp, Homo sapiens
gccctgtgttgtggcagcctcaagggcccagctcaatcctccgcaggggtcatgcagagaggccccagggctccaggcagcaggatagagagggagaaagaggggcagaaggagcagcagcatttagagaaagcttgcaaaagctggcacaacccacttccacccatcagccagatgtagctgtgagggaggctggggagwgcaggcttgattctggtggccacgcggtgaggtctgcctgagcacccttcctgcatggtacccacatgattctcttgtcacagctgtccccaaacagaatttattatgcatgtaactggtacgtccaggcatagggtggacgccaggtgcagctggatccaggcacagggaaggcgtcctcaggacccactccatcgctg

SEQ ID NO: 561 - DNA, 401 bp, Homo sapiens
cggcccttcctggcggcctcagggtctccaggtccctggactccgggcctcacccttctcctcctctccccttgccccagccctgagacggcggccttcattgagcgcctggagatggaacaggcccagaaggccaagaacccccaggagcagaagtccttcttcgccaaatacgtgagtggggctcccccgcctcccctwttcccttcctgacagtcccggtgcctggccaggccctccatgctcccacccagactcccctatggtgctcgggcctgctccttgtcctccgggcctcctgtgtgtcctggtacctgggctgaggcttgtttgcaggaagagggtatggaagtgtggtcaggggaggtcgggccagctctagggcgcagggtcctggggga

SEQ ID NO: 562 - DNA, 401 bp, Homo sapiens
ctactaaaaatacaaaaaaaaaattagcagggcatggtggcaggcgcctgtagtcccagctactcgggaggctgaggcaggagaatggcgtgaaccctggaggcggagcttgcagtgagccgagattgcgccactgcactccagcctgggcgacagagcgagactccgtctcaaaaaaaaaaaaaaaaaaaaaaagaaaaratggccagagcaggaggggagagaggaagagggagaaagaaggaaacagtgtagggggtcagaagtggaaagagaagaaaaaacagaagaggaaagaaagacgagggggtgaaaaaagggacaggaaggaagagagggagagggtgggtgccatggctggtgagctcggagaagagggaagcagagaaacgggtgaagga

SEQ ID NO: 563 - DNA, 401 bp, Homo sapiens
gacagaaagcgagagacagagagagacagtgagagaaacagacaaaatgatggcgagagagtgagaaagaaagagacagcgagagaaacagagacaatgatggcaagagagtgagagagacagagcgagagagacagagacggactgagaaaatgagacagagacagcgagagatgggagagacagggagagagagagagwgagagagagagagagagagagagagagaagcgccaaagagaagccaccaggagcgtgtggaggggaagccaggtggaactcactgaaggtgcagggagaggaaggggaggcaggagtgagaggaaagaggagaggcaaatccaagggacagaaactggaggcaaaggaggcagaggagggagggagaccaagggcacagg

SEQ ID NO: 564 - DNA, 401 bp, Homo sapiens
tggagagagagagacagagggagacggagagagagacagagggagagagagagacggagagagagacagggagagagagagacagagggagagagggagacagagggagagagagacagggagagagggagacggagagagagagggagagagagacggagggagacagcgagagaagacgcgcggagccttgcggcaggwggtcgaggaaagaacagcccctctcacctggcgtcggtggcagctgaaccctgggcccgactgtgcgatttaaggggtgccctgggggtgagaccgcccccccacccaactgccttatctggccagacccggctctgtgagctaaatcgggcagcagctgtcaccaccagggggtgtgggacagctgtccccctcccccc

SEQ ID NO: 565 - DNA, 401 bp, Homo sapiens
agccctgcctcgcccctcccagcctcctcccgggcctcctggcttctggttccccctccattctgtgcctgcagccccagaggggtctctgcacccagagctgacctgtccttcccttctcgcagctcccaggctgcccttggaggtcccgcctggttgggttcctggccgggacccaccctcctccttcatcacctcctmaaataactctggcgataaacagccgtggcgttccaggtctcatccgggatgacttccaattctggagtgccctcccgtgccccgccagcctctgcagagccctcctcagccttccgtggccagccttgtctctgggccattttatgcgacttgcttgtcccggggtcccttctctgttctctcgtccctgcagtgctctg

SEQ ID NO: 566 - DNA, 401 bp, Homo sapiens
gcgcagcggcagtgacagcagccccagcgacacgggcgagggcaggttcaggatcagccccagtacctggggcagggccagctgggacaggggcctgtgtgggcagggaggggacactgccatcagggcctgctccgcccgccggtgaaatgttgctgaacatgtggggcccaggctggtggtggtgagggcctcctgagmccacccagaggtgttccttaatcagccaccctggttcccgacactcatccgttcacctgcccactcaaacctcccacccatgcggccacccaaccagacctcccacccacccacccaccgtccacccacccacccaccgtccacccaactcacatcggcccttcaccaaccaccatcaccctgtctgtccagccacccac

SEQ ID NO: 567 - DNA, 401 bp, Homo sapiens
ctccccgtcaaccacccaaccaccatcaccatccaactagccatctactgttcaccacccaccacccacccacccacccacacagccggccaccacccagccagccattcacccaacagtggactcgccatgcccccaatcctcactcactcttgacccccctgccattggtctacccctcatccaccagccacatgcccwgccactggccagccggccatcaaaacttcccattcatccacccaactccccagcctcctgtctgctgcccattgcccaggaacacccatcacttcactccccctgacccaacacatccaacggcctactcagcattcacccagtaaccatcatccacccagcatctcagcccatcgaccacctgatctccccctttcaac

SEQ ID NO: 568 - DNA, 401 bp, Homo sapiens
ccagtgaactgagccacctcaacgatgacagctgcccgcatcaccctgatttaacactgcttgcttctcccacggggttgccctcacccctcccctgctcacagcccactctgctgcctcatttcccccaggacagagtcccacaaggccctgcgtggtctggctctggtggcctcacctcagagcctcagcaccctcctwcctggtcccctgtccccagccacatccccggtgcccaccgcctcccgccccacccccgccaacctttcctccagctcttcccctgcctaggaaggctcttccgcctgtcaccacttgtctctgggccctgcctctgaacagctgcacctctgttccctatctggaccctgggcaggcaggacatgggaggaaaacgctcg

SEQ ID NO: 569 - DNA, 401 bp, Homo sapiens
gcgatagcttcctggcgccgcgctctcatgggtgctcgcccccggcgatggcgtcatgcgcggtgcgcagacgcagcgtgacgtcacacaagggaaagggatcagagacgccaggcggtgcggggggaggcagcggtgatctttactgagtcagaagggagaccctggggaagtggggcagggcaggggtgggatggtggwggtgagaggccaggcagatgatcttgtctcgggagggaggaggcttctcagcagagatggatccccggcctggaggggagctggcagcaggctgcggggagaggggaatgagcgaatgagacagaggagagatgaggagacagacgcctgggggaggcagtggtgggaggaggggacagggcaggggggcggtggaggaa

SEQ ID NO: 570 - DNA, 401 bp, Homo sapiens
tggtcacttagagctgagggggaccccacctggggtggatgaaggaggggagggttgaggtcagggaaggatatccacttcagggtggggtgagaccaagacagaaggggcagggctctgcgtaaatggttggggtggggcttaccaggagattggcatggcgcaggatttgtgccccagattcatggattattttgggawgggccacttccacaaccagatcagggcgcctgggagaggggaaagagggcggagggtcttggagaggtattaggcctcttctccccaaggttccctccgagtctactgagggctgcccttcttctccctgacctgggaggcgaaatgagatgaatgggttcgtcctgtttcacatttgcttgaaccaaggcccagagagg

SEQ ID NO: 571 - DNA, 401 bp, Homo sapiens
accctccacgggcccctgtcggccatggccctgagtgcggtgtctggggcctggctccctgggctgctcgctgcccgctgcacaaggcctgggcagccgctgctctttggacatctgacccttgagcctctgggctcagtcttcccccttccccgcaggcccagcgtgggggcgtgggcctgcgggagtggaggcgggagwacccttccctgtgctctgtccactttcccgggtccagcacagtgagcatggtgaccgggggttctgggaaccctgcgtgcccccctttcccctgcagcacaccctctttctccctgctgatgtggaaagtttcctctgcagtcccctgccccctctctggccggccaggttgtcatggaaactgcatcttgctttgggtt

SEQ ID NO: 572 - DNA, 401 bp, Homo sapiens
ctctgtctgtcccctcgtgctcacctctcctccttgtcctcaggcggctaaggtctgagggaggcttcagcctggccgtggccctagccctcccagaggcagcgggagccctggttgtggcttctcctcaatgtgcccttgcctcagcgggtccaacctccctagccccgctgcgcagcacgggtccccttcccacagcckccgcccctgtccacagcctccctccgctctgatcatccttcagttcaccttgacttgctacctttcactctgtcccccatcgtgccaccagctgcctccagtgtgacgttctcgctcccacgtccgtccctcccgttccccagctcgcgtcagcccaggagaagccttcgaggcctggagaggccaagtgcccccaacat

SEQ ID NO: 573 - DNA, 401 bp, Homo sapiens
ctctgtcccccatcgtgccaccagctgcctccagtgtgacgttctcgctcccacgtccgtccctcccgttccccagctcgcgtcagcccaggagaagccttcgaggcctggagaggccaagtgcccccaacatgagccccagcctctgtgccttgagggtgcctggcatcggaaatggcgcaggctgcggcagcgcaggcwccctgcccccagccaccacctggtacccgctgcagtgtttccatggcaaccaggattggcgctggaaggaggctggagagaaagagagcgtgaacgggagcatggggtggggggggcgggcactgggtggcgagagccccaagttgacgggaggagagaggagacagacgcgggcagacagagaggggccaagaccccga

SEQ ID NO: 574 - DNA, 401 bp, Homo sapiens
ggaaggaggctggagagaaagagagcgtgaacgggagcatggggtggggggggcgggcactgggtggcgagagccccaagttgacgggaggagagaggagacagacgcgggcagacagagaggggccaagaccccgacgtggcacccaaaggctgccgctgctgataagggaggaattggcttccagagccaggagccccsggtgtgggagagatgagatgcctgggtgtagacagagccaggaagggggcaggggccaggcacctcacggaagtggggggcctcccgccatctctaccttctgactcctgctggggcctgagtgtccccatagtgcccatagctgccgtgtacggggtacagcctgggccactacgaggggagaagggggctgggcgcag

SEQ ID NO: 575 - DNA, 401 bp, Homo sapiens
ctccccttcggcaccccgtcctctggagcagccttgagggggtctgcagtggagagacacactctggattgggccctgttgtgggttgaattgtcacccagaaagggaagcgcagaagtcctgacctccagggcctcagaatgggaccttatttgggaattgggtttttgccaaggatggagctgaggttatactggaggwggaggctcctggtccagtctgacctctgtccttgtaagaggggaggttaggcaggctgcagtggcccacacctatagtcccgacactttgggaggctgaggcaggtggatcacttgaggtcagaagttcgagaccagcctggccaacatggtgaaaccccgtctctactaaaaatacaaaaattagctgggtgtggtggt

SEQ ID NO: 576 - DNA, 401 bp, Homo sapiens
cccttcggcaccccgtcctctggagcagccttgagggggtctgcagtggagagacacactctggattgggccctgttgtgggttgaattgtcacccagaaagggaagcgcagaagtcctgacctccagggcctcagaatgggaccttatttgggaattgggtttttgccaaggatggagctgaggttatactggaggtggwggctcctggtccagtctgacctctgtccttgtaagaggggaggttaggcaggctgcagtggcccacacctatagtcccgacactttgggaggctgaggcaggtggatcacttgaggtcagaagttcgagaccagcctggccaacatggtgaaaccccgtctctactaaaaatacaaaaattagctgggtgtggtggtagt

SEQ ID NO: 577 - DNA, 401 bp, Homo sapiens
gaagagatgagaggctacaggggcaggcactggaagactggggtgatgtgatacagccaccagccaggacagcctggggacaccagaagctggcgaggcaggaagctcccctgaggtatccccaggtttcagagagagcgtggccctccctccagcctccagaactgtgggacgatagatttcctttgttataagtctccrcctgggtttgggtgtggtgtggcggtgctgggggaggagtttaggaagaaagggaatcagaccttctctggggtctccagggtgaaggctgaggggcctaagaccacccggaggtgccccgtgcccaccctcccgacggagctgcgaggctggctctccagcctctccttccgcccccacccacattcactttgttttct

SEQ ID NO: 578 - DNA, 401 bp, Homo sapiens
gggggctccacgcccctcgcccgcccggccccgccgcctcagatctgcgtctcttgcacgttctccttggagccgctcttgaagagcagaggttcgtggatggagttgaggccaggcgggcctttgcccccgcagcccccgccgctggggttgctgctgtagtgcgccttgaaggcggcagccacgtagtggtggtggttsaggtggtctcgctccagggcgggcagggccaggtggctgtccccgcccacaccacccccactggccacggcggccgcggcggccacggacacggccgaggcggcgggcagctcgtcctccacgttgatgatctccacggtgcgcgtgggcccgtggtgcttgtggagctggtgctgcttgcgcagcttgtagaaggccac

SEQ ID NO: 579 - DNA, 401 bp, Homo sapiens
tctccgtcccccgcggctgcagggcctcctctccgggctgcgtctccagggtctccacggtcaccgtggtgaagtaggtgtagccgccactgccccctccaacaccaccactgcccccagggccgcccccgccgctgccggtgcccccggccgccacggggtccacggccgagacgttgagcgtggccgaggcggtggtgktgccggctgagttcgtcaccatgcacgtgtactggcccgtgtcctgcacggtgacgttggtgaagttaagcgtgccgtcatgcaggacggagatgcgcacgcggtaggagccgtgggtcatgagggtgccgttgggcgtcagccagttgacggaggtcatggaggtgcccgtgcggcatttgagctcggcagccatgccc

SEQ ID NO: 580 - DNA, 401 bp, Homo sapiens
tccgtcccccgcggctgcagggcctcctctccgggctgcgtctccagggtctccacggtcaccgtggtgaagtaggtgtagccgccactgccccctccaacaccaccactgcccccagggccgcccccgccgctgccggtgcccccggccgccacggggtccacggccgagacgttgagcgtggccgaggcggtggtgttkccggctgagttcgtcaccatgcacgtgtactggcccgtgtcctgcacggtgacgttggtgaagttaagcgtgccgtcatgcaggacggagatgcgcacgcggtaggagccgtgggtcatgagggtgccgttgggcgtcagccagttgacggaggtcatggaggtgcccgtgcggcatttgagctcggcagccatgccctc

SEQ ID NO: 581 - DNA, 401 bp, Homo sapiens
cccccgcggctgcagggcctcctctccgggctgcgtctccagggtctccacggtcaccgtggtgaagtaggtgtagccgccactgccccctccaacaccaccactgcccccagggccgcccccgccgctgccggtgcccccggccgccacggggtccacggccgagacgttgagcgtggccgaggcggtggtgttgccggmtgagttcgtcaccatgcacgtgtactggcccgtgtcctgcacggtgacgttggtgaagttaagcgtgccgtcatgcaggacggagatgcgcacgcggtaggagccgtgggtcatgagggtgccgttgggcgtcagccagttgacggaggtcatggaggtgcccgtgcggcatttgagctcggcagccatgccctcggtga

SEQ ID NO: 582 - DNA, 401 bp, Homo sapiens
tgaggtccgtgggcggctccacgatgacgggcgcatagcaggtgaaatgcgactggtccagctccccaatgtagcgccccttgaggccggcgggcgcatgacagcgggcgcagcacgtcgtgttgctgggcaccgtctccttgagccaccagctcagccagagcacgtcgcagttgcaatgccaggggttgtggttgaggwgcacgcgctcgaggcggtgcaggggcgtgaagaggtcgtggggcagcgacatcaggttgttgtgggacaggttgagctcctccagcgacttgaggtcgtcgaaggcgttgcgctcgatggtggctacctgggcgtgcatgagccacagcttgcgcaggctggtgagaccctggaaggagcccgggcggatcaggtccagc

SEQ ID NO: 583 - DNA, 401 bp, Homo sapiens
accagtctgggcaacattgggagaccctgtctctacaaaaagaatttaaaaagaaatataaaaccaagaattttgggattgaagaggaaaaaacttttgaaaaaaaagttaaaactggagtggccatccgacccggctgttccattcctgggtatttcctcaagagaaatgaaggcagatgtccacgcaacggcttctatktgggtgttcacagcatttttttctcaggactgcctaagctggagacatcccaaatgttcctcagcaggtgaatggacaaacactgcgaggtgtggccgtgtaaaggatgctgcggctcagccatgaaagggacctggctgctgtgaccccctggatgaatctctcagctcagccatgaaagggacctggctactgatgac

SEQ ID NO: 584 - DNA, 401 bp, Homo sapiens
ggacagcagagttgggactggatccaggccctctggttccagagtcctggctcttgacctctgaccctggctgccgcctctcagggctggagctggggtccacgggaggggcagtgctgggtcagagcacgggaggcaggagctcacagggtcctcactactgatgaccacgccgggtgcactgaggcccagaaggccacwcagccattggatcatatctgcaaagcccagatagtggtgtgtgccagggagaaggggacggacactgggggaatgcggtgttacagtattaacggaggaaactgagaacctcagatgcatcctgtgtcatcacaaagaagctgaagaattaggtatcagagtatatgcaccaaaagaaaaaagagaaaaagaggccgggc

SEQ ID NO: 585 - DNA, 401 bp, Homo sapiens
gatggggtcttgctgtgttgcccagcctggtctcaaactcctggccttgagcaatcctcttgcctcagcctcccaaagtgccgggattacaggtgtgagtcactatgtctgtgcttgatttcttctttcttttttttagatggaatttcactctgtcgcccaggctggactgcagtggtgcgatctcagctcactgcaacytctgccttctgggtttaagcaattctcctgcctcagcctcccgagaagctgggattacaggtgtgcaccaccacgcccagctaatttttgtatttttagtagagacagcatttcgccatgctagccaggttggtcttgaactcctgacctcaagtgatctactcgccttgccctcccaaagtgctgcgatgacgggcgtg

SEQ ID NO: 586 - DNA, 401 bp, Homo sapiens
cttgcatttcttcctcagagagagtatctggggaagaacccaacctggagcttctcagagccggttctcagagctcatttcccgggacgctaaacaccaacagcaaaaaaacactctctcatatcaatgcgttaggagcctgcacactccctccccgagactccagtgcacctggcgtgttgaagctccgtgaagcccagygggagacagcggctttgttttgctcaactctagttttcccaaacataatacacatggggcccttctcaccaaacgcagcttatcacctccctgggaaatgctggtgattgataaagcaggcactcggttgctatttctattaataataataacatcaataatagtaacaggaggaggaggtcccggtcatcactgatcgc

SEQ ID NO: 587 - DNA, 401 bp, Homo sapiens
agttgagaactgagtggaatgggggtgttgctgcaaagggaagcctggttttgttgaaacatgtggcatagggctgctttaaatagggagtctctttattttgaacagaattccagtctattctgtctcctcttctctgcagaatgcctgctgagcagcctcgtgggggatggttgaggctgggtttgaatcctggctttkctgctgaccagcctgaactccctggtggcctcgggcaagtgtcctcacctctctgagcccactttcctcccaggtagcatggggttactgtgatgcttggacaagacagtgagcgcttgggatgagacaatgaatgtgtgggacatggcgagcatgcagtaatagatattttttcattgttaattcagttgataatgatc

SEQ ID NO: 588 - DNA, 401 bp, Homo sapiens
ctgatttgaaacaccctagaccagatcacctctgaagttgtgagtgtcatcaccctcatggccctgacacagagtaggatgggcaaggtggggattggttcttgacgcaggtaaatcctcctgccccctcaccctaacatctttagcctgacgaagatgttgacattttgtcctacagtagaggacctaggcctggggccmcctctgacagccacatagctgcccacacttccaagttccctggatcttcagcccccatctgctgagcccacctccacctatgctgggctctgggacagatctcaatgatgcagatgtgaatccggcaaagggacgtggcccccatcaatgactgccttctaaccccgatggggactttccttttatcctgcaacttggcc

SEQ ID NO: 589 - DNA, 401 bp, Homo sapiens
tgaaacaccctagaccagatcacctctgaagttgtgagtgtcatcaccctcatggccctgacacagagtaggatgggcaaggtggggattggttcttgacgcaggtaaatcctcctgccccctcaccctaacatctttagcctgacgaagatgttgacattttgtcctacagtagaggacctaggcctggggccacctctkacagccacatagctgcccacacttccaagttccctggatcttcagcccccatctgctgagcccacctccacctatgctgggctctgggacagatctcaatgatgcagatgtgaatccggcaaagggacgtggcccccatcaatgactgccttctaaccccgatggggactttccttttatcctgcaacttggcctgtgta

SEQ ID NO: 590 - DNA, 401 bp, Homo sapiens
gaaacaccctagaccagatcacctctgaagttgtgagtgtcatcaccctcatggccctgacacagagtaggatgggcaaggtggggattggttcttgacgcaggtaaatcctcctgccccctcaccctaacatctttagcctgacgaagatgttgacattttgtcctacagtagaggacctaggcctggggccacctctgmcagccacatagctgcccacacttccaagttccctggatcttcagcccccatctgctgagcccacctccacctatgctgggctctgggacagatctcaatgatgcagatgtgaatccggcaaagggacgtggcccccatcaatgactgccttctaaccccgatggggactttccttttatcctgcaacttggcctgtgtag

SEQ ID NO: 591 - DNA, 401 bp, Homo sapiens
agatggggtttcactacgttggtcaggctggtctcaagctcctgacctcaagtgagcacccgcctcagcctcccgaagtgctaggattacaggcgtgagccactgtgcccggatgagacttgccgactttctgaacgacctctttctgcacgcagtgtcgagagggcagggatctctgtatgtcctgctctctgggcctcrggtggtgcctggcacacactaggtgctcaataaacaaatcagggctgcactgaatgctgcagatggattagccggggctgttcagacacaggagggacagcccatgcagggagaaagatgcacgggtctcagctcagctcctagactcgccacggggtggtgacgagaccacggcatccttgtgaggattaaactgtctc

SEQ ID NO: 592 - DNA, 401 bp, Homo sapiens
ctctaactgggaaaagtctgtccaatcaaaagctccatgtttcatgcccctggagggatcagccgcctggaaaatgcgaactagtgggtaatttaaaagctattcttggtatttgccttgataatgcactatggccggggtttttatttggttagtttgggttttgtcttgtttttttttttttttttttttttttttgakacagagtcttgctctgccacctaggctggagtccagtggcaaaatcacagctcactgcagcctctgcctcctggcctcatgcaatcttcccacctcagcctcccagggatctgggactacaggtgcgtgccaccacacctggctaatttttgtatttttagtagagatggggtcttgctatgttgcccaggctggtttca

SEQ ID NO: 593 - DNA, 401 bp, Homo sapiens
agaccagcctggccaacctggtgaaacctcgtctctactaaaaatataaaaatttgctgagcgtggcagcaggcgcctgtaatcccagctactcaagagactgaggcggaagactcgcttgaacccgggaggtggaggttacaatgagccaacgttgcgccactgcactccagcctgggagacagagcgagactctgtctsaaaataaataaataaataaatacaaaaataaagagcctctgattaccctcgacgtggtccatgcctcttgctgtctgggatgtgtccactgagggaggccaagcccactgacaaacactcacaagcatccactctcgtggactggacgaacacaccctccatccatgtctctgggtggggcagggggcggtgcaagaacc

SEQ ID NO: 594 - DNA, 401 bp, Homo sapiens
ggccgacccgcctgtcctgtgccgggggggctgggcaggtggcctgcacttgggccccgacacggtggctcagagacagggagcactgtgctctcaaagcccggagggcctccactccacctcgggagcccggctccagctgataggatgcagacccgcaggccacatccacaggtgccggagacggggaagccccggtgkggggaggcccagaagggatgggctacatctgcatgcctccccagtgtcccctccaaggtccccctctgcccgctggccccgcatacctggatgccgttctcttgcaggttcaggtaccgcgtgttgaccgggatgctggctgggacctcggccaggtctctccgtgtgcagatcacccggctggcctggttgctgcagga

SEQ ID NO: 595 - DNA, 401 bp, Homo sapiens
accattttaacccagataaaaatgtgtgtgcatacgtctgtgtatctgagtgcatgtgtgagagcgagggctagacagagacacagagagacagagacagaagcagaggcagagagacagagggcaggggaaagagggagctggggagagaaagagagagaggttgattctgtgtccacccgggctcatttttctcctgcwgggctggggccgtgtgtctgtgtctgactatgtgagtgcggtgagggtcttcattttcctgaatgtgaacagggaggtcaatgcccagtccttatcctgggtcaggggctcctacctcttaagaccctggcgatgatccagcccacaccctcttagccaaagggctggcccaaagcgcagggcctggatttgaggccggg

SEQ ID NO: 596 - DNA, 401 bp, Homo sapiens
ccttatcctgggtcaggggctcctacctcttaagaccctggcgatgatccagcccacaccctcttagccaaagggctggcccaaagcgcagggcctggatttgaggccgggggaccccgcagggcctgtgggagcccacggcacccaccccggcctccaggcctgccgctgccatgtctacctccgcatctgtccccagcyctcccgctgccagcccgcatccccccacccccatcacggcttctgcagcgactcggcctccctccccttccccccgccgccctccgccatctcccatccttttctccccacaaccccacggtgggggacactcaccctggtggcttggcttccgctgggcctctccctgcgcccaggcccccccctccacatgcctccgg

SEQ ID NO: 597 - DNA, 401 bp, Homo sapiens
aatttctagtcacacggcagtgcagagccaagctgtgcctcagctccctccctccccacccctccctccttctttctctccttctgaacggggagggcgcctctcctccctcctcctcagcctcccttccctgcgccgcctgggggaggcctccccctcccccgctaatcaaaggctccgttatctaattaacccattggktctgggcagccgaggaggcagccgaggaaggtcgggggaagcgggggaggaaggttgccggctgggctgcccaagaaaggggggtggttgggccgtgccaagccggagcctcatggcacacagacagcctcgttggctgcatgaccgggctgggccaccactgaccccctcgcccccacttccagccaaaaccccccagc

SEQ ID NO: 598 - DNA, 401 bp, Homo sapiens
cctccactgtgaaacagggataatgggagtatctgcctcctagggccactgtggggattaaatcgctaaatccttggtaaagagttctgtcttcacaacatatgcggaatcagaccccagaatcagaggatctttctccccacgcccaggctacttccctgacctggctcccatctccctcttgaacgattacagtgtccwcccctccagcctccccatttctgccttcacccccactccccgtgcgttccccacgggcagcgaggggtatcctgtcaacaccccatggaccctgccccgcctctgctccacagccttccacagctcccgtcccactcagagcagagatgaggacttcagttttcaggtctccactctccaccccagctcctcctctgacc

SEQ ID NO: 599 - DNA, 401 bp, Homo sapiens
actgtgaaacagggataatgggagtatctgcctcctagggccactgtggggattaaatcgctaaatccttggtaaagagttctgtcttcacaacatatgcggaatcagaccccagaatcagaggatctttctccccacgcccaggctacttccctgacctggctcccatctccctcttgaacgattacagtgtcctccccyccagcctccccatttctgccttcacccccactccccgtgcgttccccacgggcagcgaggggtatcctgtcaacaccccatggaccctgccccgcctctgctccacagccttccacagctcccgtcccactcagagcagagatgaggacttcagttttcaggtctccactctccaccccagctcctcctctgacctcatc

SEQ ID NO: 600 - DNA, 401 bp, Homo sapiens
gtgaggccgtcctgaccgccccgcatcaaacagcacccctatccctctccagaaccttctctgtttcctttttctccatgacatttatcagcatctgagcttttatatgggtccgtgcgcttgaaggcaagtttcccgagggcagtcagtttgtgactatttggttcccaggacccacaacagtgctgggcacatggcagktgctcaccaaacactgggtgcagggacggatgggtacgttatcatcactgtgagtctatttccccccacgctcacacacagatacccccactctccgttctgcacaccgagccaggccgcactgcaatagctgcacaatgcaggctcccagccctggacaccctcggtccccaggtccccttccatcccaggaggcctgc

SEQ ID NO: 601 - DNA, 401 bp, Homo sapiens
cagccctggacaccctcggtccccaggtccccttccatcccaggaggcctgctggcagaggcagcaccttttttttttttttttttttgagacagagtctcactctgttgcccaggctggagtgcagtggcgcgatctcagctcatggcaacctctacctcctgggttcaagcaattctcctgccccagcctcctgagtarctgagactacaggcgctcaccaccaccccggctaatttttgtattttaagttagaatcgggggtttcaccatgtgggccaggctggtcttgaactcctgacctccagtgatccacctgccttggcctcccaaagtgctgggatgacaggtgtgggccaccgtacctggccgaggaagcacacatctcacgtcagctggac

SEQ ID NO: 602 - DNA, 401 bp, Homo sapiens
gaatgtcgagctgttacagaggccgtggcaatgggccgggggcgggggggccctccttcctctcacaacgggctgtgacaacagaggggcaggctcagctctggtgggtgaaggacccctctgagtggactggagcggcgcggctggaggccagggaggagctgcgccaggggtccgggggtagaggatgaggccgcggcyggagccacggggatggagagaagggaaagagtgaacagataaagcaaatttgtaaaagtggcagaacctggaggcggtgtaggaggtgagggagagggaggagccaggctcttcttatagatctggggtcaaattccaaaagccagaagaggatgtggggcgggaagcagttacggggagaagagaatgcaaaagtccct

SEQ ID NO: 603 - DNA, 401 bp, Homo sapiens
ctcagctctggtgggtgaaggacccctctgagtggactggagcggcgcggctggaggccagggaggagctgcgccaggggtccgggggtagaggatgaggccgcggctggagccacggggatggagagaagggaaagagtgaacagataaagcaaatttgtaaaagtggcagaacctggaggcggtgtaggaggtgagggwgagggaggagccaggctcttcttatagatctggggtcaaattccaaaagccagaagaggatgtggggcgggaagcagttacggggagaagagaatgcaaaagtccctgccacaggccccccagccccaatctccactcacaggcggtgcctcgctctggtcacacccacagggcagcaggggagctggggacacaccgcc

SEQ ID NO: 604 - DNA, 401 bp, Homo sapiens
caacatggcaaaatcccatctctactaaaaatacaaaaattagccaggcgtggtggcatgcaccggtagttccagctactcgggaggctgaggcaggagaattgcttgaacccaggaggtggaggttgcagtgagccaagatcgtgccactgcactccagcctggatgacagagcaagactccgtcttgggaaaaaacaaraacaaaaacagagcatcacttacgacgttttcttgccaagagcatttaaagtaaatctaattaagccccaagacctatggttcccaaactagatgccaagacaccctggggggactacagcaaattcatagcaggccacagcagtgccatgcaggtaaacgtttaccagccggttctcaacattcgctagagaagagtcg

SEQ ID NO: 605 - DNA, 401 bp, Homo sapiens
aggtgctgtgaactgagaatgtgggggatcctctgcacgagctctcttgtccatctacaggaattaggagagagaaacaagctggaagacactgcgaaggaacaatcaggcaaatctagaatgtggggcattctaggagacaaccagcctggtccctccaaaaagtccccatgatggggcagagaaactggctggagtggytgctctaagttaaaatagattaaggagatacaaccaccacatgcagtgcttgagccttgcttggatcctggtttgcaacatttcttttttctttttttttttttttgggagtgggggtctcacttttttgcttaagctggagtgcagtggcacaatcatagctcactgcagcctccaactcctgggcttaagtgagcctc

SEQ ID NO: 606 - DNA, 401 bp, Homo sapiens
tcaaagcactaggattatagatgtgggccactgtgcccagcccatagtatgttttaaaagtcctaaaatacaatgtctggacaagtgggataatgtgaatttggactcgatcttagaagcactagggaattaagtttactcttcttgggtgtgatatgatataatattgtagttatgtaggagaatgttcttattcttttstttcttttttttttttttatttttgtttttgagacaaggtctcactctgttgcccaggccggagggcagtggcacagtcttggcttggtgtaacctctgcctcccaggttcaagccatcttctcttttcagcctcctgggtagctgggactacaggtgtgcaccaccatggctggctaatttttgtattttttgtagagt

SEQ ID NO: 607 - DNA, 401 bp, Homo sapiens
gatcacattaacctccaacttaaaatactcggaggacttctcactgcatttgggattcattcctccttcccatgactgctaaagtcctgagcggtctgggccttgatgctgccccaccctcacctcccacctctccccactccagcttctccaggacagtgtgctcattcctaccacagggccttggcacctgttcctctmtgccggaacgctcttctcccacatcgtcacatgctggcttccgattttcaggtttcagttcaaatatcacctcagaggggccttccttgtcctgctgatttagagacactctcctacctgttcttggtaactctttgcaacacaattgtgttctgttttcttcacagctctcattgataactgaaattttcctcttttct

SEQ ID NO: 608 - DNA, 401 bp, Homo sapiens
aggaggtcgagaccagcctggataagatggcgaaaccctgtctctactaaaaatacaaaaattagctgagcatggtggcacgcgcctgtagtcccagcttctcaggaggctgaggcaggagaatcgcttgaaactgagaggccgaggttgcagtgagccgagatcacgccactgcactccagcctgggtgagaagagcaasactccatctcaaaaaaaaaaaaaaactattataaaatatcaccatgaacctctttgtaagcttctgactttcaggtgaattcccaggcatgggatattttcagggtatggacattgttcctgtatttgacacctttcaccaatttgctttccaaaattgtggcaatttaccttctgacaactgagtatcagcatgcatgt

SEQ ID NO: 609 - DNA, 401 bp, Homo sapiens
cggttaatcttttaaaaattactgatttgatttgtttcatggttttggactaactgaatgctatattttatcttgagtcatttttggtaagttatagctttccaggaatttgtccatttcagctatattttaaaatttgttagcgtaaagttgttcttaataaagttccctcttaaaaattcctgatattggctatttgtrcttttttctttctctttttaatcactatcactggaggtttgactatcttactaggcttttcaagtaaccaactttggtgtagttgattatcctaactctatgtttgttttctatatgacttttttcatttttattattttttccatttccttatttttttggatttttttagttattctttttctaactccttgagatga

SEQ ID NO: 610 - DNA, 401 bp, Homo sapiens
ttgaagttctgctgtgttgagtctgtctatcagcaccattttaacaacagcgtgtgctcccttaatgtctctgtgtcacattttggtaattctcccaatatttcaaactttttctttatcattctgttatggtgatctgtgatcaatgatttttttttgagatgcagtctcactctgtcgcctaggctggagtacggcggygcgatcttggctcactgctgctccgcctcccgggtccaagtgattctcccgcctcagcctcttgagtagctgggtttacaggcgtgcaccaccatgctcagctaatttttgtatttttagtagagacggggtttcaccatgttggtcaggctggtctcgacctcctgacctcgtgatctgcctgccttggcctccggaag

SEQ ID NO: 611 - DNA, 401 bp, Homo sapiens
actgagatcacaccactgcactccagcctgggtgacggagtgagactctgtctcggaaaaaaaaaagaaaaaaagaaaaagaagtgcaaggtgaagcaccaagtgctgatggagaagctgcagcaagttatccagaaggcctggctaagataattgatgaaggcagctatactaaacaacagattttcaatgtaaatgaarcagcctacagccttctatgaaaagaacatgccatctagggcttttatagctagagaggaaaattcaatatttggcttcaaagttccaaaggacaggttgactctcattttaggggttcatgcagctgatgactttaatcgaagccagtgctcctttgccattctgaaaattctaaggcccttaagaattttgcagaatct

SEQ ID NO: 612 - DNA, 401 bp, Homo sapiens
tctctgtcccttctgtcacagtcctccctgtttctattactgtccccttggtatctctcccatcttcctccttcctcttcttcccctctccttctccttctccttccccttctccctctcctctccctctccttcttctctctttctctcactttttctttttttttttttcttttgttgttgttgctttgtgtttttttkacgtagtctcaccctgtcgcccaggctggagtgcagtgacacaatcttggtaaactgcaacctttgcctcccgagttcaagcgattctcctgcctcagcctctccagtagctggaactctccagtagcaggcactacaggcgccgccaccagacccgcctaatttttgtatttttagtagagacggggtttcaccgtgtt

SEQ ID NO: 613 - DNA, 401 bp, Homo sapiens
aaaaaaaaaaaaaaaaaaaaaattagcagggtgaggtggcacacgcctgtagtcccagctacttgggaggctgaagcaggagaatcacttgtactcagggagggggaggttgcagtgagccaagactgcaacactgcactccagcctgggtgacagagtgagactctgtctcaataataacaataataataataatgcaasattttaaaaaaaatcaaaattaatgcaaaaaaatcaatgatgaagaaaatatcaaatttttaaaaaaggccagtctcatgccacatcagactgctatttcaaaggaaatgtatgtgccctcatatacttgctttttttttttttttaaggagaggggttcctgctccattgcccaggctggagtgtggtggtgagatcat

SEQ ID NO: 614 - DNA, 401 bp, Homo sapiens
tgatagttacctagaattagagtggaggggcagagaccagagagcgagagagaaacctagactcagataccctactccctgcaccaagagaaagagagagagagagagagaggctgactcagacagagtgggacaaagaccaagcagacacacacacataaaccagcctgagagagggcaggagattcatcaacctccacygaagtttccaggactgcccccaaccctgacacacaggctggagccacgctggagctagctgggagctttgaccaacctggcctagccctgcggcctcccaactgtgcccaggactccctactcattcccctcagggcatctgcccaggctgtccctctgccagaacactcttccctctatctctccttggcctttgagcc

SEQ ID NO: 615 - DNA, 401 bp, Homo sapiens
ttccccccatcccccagccaggtggccacaagtcccctcctttgctcactgtgtgagcttgaggaagccactgtgctctctgagcctcagattccccattggtatctggggggcattccttctctccctccctccctccctccctcccttctttccttccttccttctttccttttcttttgagacgaagttttgctcttrtcacccaggctggagtgcaaaggcgcgatctcggcccactgcaaactctgcctcccaggttcaagtgattatcctgcctcagcctcccgagtaactgggattacaggcatgcaccaccatatccggctaatttttatatttttagcagagatggggtttcaccatgttggtcaggctggtcttaaactcctgacctcaga

SEQ ID NO: 616 - DNA, 401 bp, Homo sapiens
cattggtatctggggggcattccttctctccctccctccctccctccctcccttctttccttccttccttctttccttttcttttgagacgaagttttgctcttgtcacccaggctggagtgcaaaggcgcgatctcggcccactgcaaactctgcctcccaggttcaagtgattatcctgcctcagcctcccgagtaackgggattacaggcatgcaccaccatatccggctaatttttatatttttagcagagatggggtttcaccatgttggtcaggctggtcttaaactcctgacctcagatgatctgcccgcctcggcctcccaaagtgctgggattacaggtgtgagccaccacacccagcctcattctccccatttcaaagctagctccagccc

SEQ ID NO: 617 - DNA, 401 bp, Homo sapiens
ggcgccaggatgggaaaagcattttaaagaaaagcattttagacagtaggagcaggatgacaggggcttagggtaggaaagcagttggagggtccagggaccaaggaggccagactggggtagacgcggggagggccaggagagaaggatggatgtgggtagagagattggaggtggataggtatggtgggagctgggggyggagaggtggagatgcagagatggggaccaagttgagggagggagagatacagggaggggagacagagataatgtgaagggcagactgaggctccgaagagatggagaggtgaacaggcaggcagaaggaaagtcaaggattgagacttgggatggacagtgacaaggaatgaaccgagatgtaattaattcacgggggc

SEQ ID NO: 618 - DNA, 401 bp, Homo sapiens
tgggcagaaatatctttgaaagagaaacggtcgaacgggaggatggatgggtgggcaaggacagcagatgatggctagagatcgcagatagacaaaaggagacaggagggctgggcagacagacatgggttcataagggaaggttcaatgagcacataaatgagaggaggctaggaaggagaggtggatgtggggagaggrggaattagtgacacatggctcggagccaggcatcaagatgggttgaaggggatgcaggggaattaggaacagacagaccagggctcacagatggacaggtgtcgtgggggtgctgcagaaggccaggactccaagacaggcatggccacactcacctgcgcttatctctgccgtgtcctgggtggcagccgaggcggcgg

SEQ ID NO: 619 - DNA, 401 bp, Homo sapiens
gggagattgagaacacgcacactggatccagatgcttacatttttatccttattctatcattttgtgctgtgtgactttgggcaagtcacttaacctctctgtgcttctgtttcatcatgtgttaaacaacactacctcggaaggttgttgtgaggatgcaatggaaagtattttattctattctattttattttattttsgcactagagtcttgctttgtcgcccaggatgcagtgcagtggcacgatcccggctcactgcaatctctgcctccagggttcaagcaattctcctgcctcagcctccggagtacctaagactacaggtgcgcgctaccatgcccgcctaattttttgtatttttagtagagatgggatctcaccatgttggccaggctggt

SEQ ID NO: 620 - DNA, 401 bp, Homo sapiens
ggagattgagaacacgcacactggatccagatgcttacatttttatccttattctatcattttgtgctgtgtgactttgggcaagtcacttaacctctctgtgcttctgtttcatcatgtgttaaacaacactacctcggaaggttgttgtgaggatgcaatggaaagtattttattctattctattttattttattttgscactagagtcttgctttgtcgcccaggatgcagtgcagtggcacgatcccggctcactgcaatctctgcctccagggttcaagcaattctcctgcctcagcctccggagtacctaagactacaggtgcgcgctaccatgcccgcctaattttttgtatttttagtagagatgggatctcaccatgttggccaggctggtc

SEQ ID NO: 621 - DNA, 401 bp, Homo sapiens
attgttagagccgggctgtgtcaggtgactttggggagggttcacgggggtggggctttgctctggatgggagactgatggaaagcacaggcaagcccatgactgggaatcttcatctcattgggaaccaggaggggtagatggagctaatgctggggttaatgaaaaagcagccgtcggctctgagctgggagggggggrttttggtcatttttgtggtttggacaatgttcatgtttgttgtgttgagacatgattcaggggtggtcttgtttttgtctcagtccatcacggtcacagagtggccttgcttgatgttggtgttacgggaaatgttttatgttcggcaggagagcaccagggccgggctgtcagtgtctgcttccctccctttcagcagc

SEQ ID NO: 622 - DNA, 401 bp, Homo sapiens
ctggtcacacctagatcgacgtagtcctccttggtcaaggcgggcaggtgggagccatcgatctcgtggtccaggaactgggctcggtgctccgccaaacccagccactccagccaatcagccacgtcgaacttggtccagaaccccagaggtttagcgccaaacggcttgtccgggggcagcgagagcaggcgggtcggkgagagggagcgcgaggcccctgacaaggctcccccgagccccccggatatccccgggtgtggcggcacaaagactggggcgaaggggtcagccgagcctcctgcccctccagttggggagccacggatgtcaaagaggcctgggtagaggggtccggaaggcaggatgggcagggacgagggcctgggcgcagggctgac

SEQ ID NO: 623 - DNA, 401 bp, Homo sapiens
gcctcctgcccctccagttggggagccacggatgtcaaagaggcctgggtagaggggtccggaaggcaggatgggcagggacgagggcctgggcgcagggctgaccttgtgctccgaggcgggcagcagcgaggggctgggggcccggcggagcagagggggccgcatctcgaactccacgccctggaggtggcgggtggwcgtggaggaggaggaggctgagggtgaggtggcccctggggccgcagcggctgtaggggagacccctgttccggtggggagtggcggcagaggtggtttgggccagttctgaaacaggctgctgacaggcttggagaggagtgaggactgggagtcgtcggagagtctggaatgtgacaagggggcagtgggggaggaca

SEQ ID NO: 624 - DNA, 401 bp, Homo sapiens
agcctgaccaacatggagaaaccccatctctactaaaaatacaaaattagctgggtgtggtggcatgggaggctgaggcaggagaatcgcttgaacccgggaggcagaggttgtggtgagccgagatcacgccattgcactccagcctgggcaacaagagtgaaactccctctcaagaaaagaaaagaaaaaaaaaaaaakaaaaagaaaatatttgtgaaagcatgcttctgtgacagcaaactcctgccaacctggtttctgtttcctttctgccaccctgagaccctaaagggacacagccatgccatcaacacctctggcctcccttgttggccaccagcccccgcttacctctgccgctgcagggaggaggtcctctcggggacatagctggctcc

SEQ ID NO: 625 - DNA, 401 bp, Homo sapiens
tgaccaacatggagaaaccccatctctactaaaaatacaaaattagctgggtgtggtggcatgggaggctgaggcaggagaatcgcttgaacccgggaggcagaggttgtggtgagccgagatcacgccattgcactccagcctgggcaacaagagtgaaactccctctcaagaaaagaaaagaaaaaaaaaaaaagaaamagaaaatatttgtgaaagcatgcttctgtgacagcaaactcctgccaacctggtttctgtttcctttctgccaccctgagaccctaaagggacacagccatgccatcaacacctctggcctcccttgttggccaccagcccccgcttacctctgccgctgcagggaggaggtcctctcggggacatagctggctcccccg

SEQ ID NO: 626 - DNA, 401 bp, Homo sapiens
cttgaacccgggaggcagaggttgtggtgagccgagatcacgccattgcactccagcctgggcaacaagagtgaaactccctctcaagaaaagaaaagaaaaaaaaaaaaagaaaaagaaaatatttgtgaaagcatgcttctgtgacagcaaactcctgccaacctggtttctgtttcctttctgccaccctgagacccyaaagggacacagccatgccatcaacacctctggcctcccttgttggccaccagcccccgcttacctctgccgctgcagggaggaggtcctctcggggacatagctggctcccccgtggtggctgtctccgcctcccccactgcctcctcgggcccagggcagagcgccgccagctgccgaggagcccccaaactgctgaa

SEQ ID NO: 627 - DNA, 401 bp, Homo sapiens
gcaggaggggccccaccccgggagcggtgggcggcaggcggtgtagcagggaacgccgggcggcgggcgggctggcgggaagggacttctcctggctgcccagcccggccctgtaccccagctcccggcgcgcagggtccggcggggaggccccccagagacgcagcactggctcgtggtgggcgtggggcgagtggtggygcggggtgggcggcggggcctcgtagcggggcgatgggggcctgggaggcggctggggggccccgccgccctccgaggactccttcagcgctcgctcgcgggcggccagggccagccccagcggggaggcgggatccagggccttgccggtcagcgggtgcaccaggggtcgcgggggcaggaagctggtgaaggcgctg

SEQ ID NO: 628 - DNA, 401 bp, Homo sapiens
cagactcctcccagcccacggtccctccccaccaccgcagcaaacagcagctctgtcctttcaggtgctaagactgaaaaccttgcagccactcttagctttcttcctcaagtcacccacatccaatccaccagcaaatcctgttgtttcaaaacatatccggaatccaatcccttctcacccctccagagtcaacacccygggccagccactatcatttcctatctggatagcaatagctgtcacctcctctttggtcccccggcttcccagctcatccccttcaatctagtcccttcacagcagccagagggtttctgttaaaaccgaagtcagctcatgtccctcctctgcttagagccctgccattcactcacctcactcagggcaaaggttcaagt

SEQ ID NO: 629 - DNA, 401 bp, Homo sapiens
ccaagtctgcttgcttttcctgaactgtcacgttcctggcctcccccagcccccacatctcttcttctgggtggcataccgatagatttttgccggagcatcaacccaggtcctggaggcaggaaagaaggacgctcgtaacttggctgggcacggtggtggggatcgaagctcgactttggagcggcagtcagagggggwgagagagaaaagatagagagagatcagtcagcgcagaaaggcttgtggcttcactatacggctgcccccattccttccctaagcccctgccgtaagggagagcagtgtgaagacggtggaaggaggccgggcaacgtggctcacgcctgtaatcccagcactttgggaggctgaggcaggaggatcacctgaggtcagga

SEQ ID NO: 630 - DNA, 401 bp, Homo sapiens
ctactaaaaatacaaaaattagccaggcatggtggcaggcgcctgtaatcccagctactcgggaggctgaggcttgagaatcacttgaacccatgaggtggagaggtggaggctacagtgagccaagatcacaccactgcactccagcctgggcgacagagccagactctgtctcaaaaaaaaaaaaaaaaaaaaaaaaakaaaagaaggcgaaagggaattctgcgggaatgaaaagcctcaagtctgggcaggaaggagaaagaaaggagaagaaagcagattttccatcgactcttgatctgcttccttcctccagccactgcccacatccaggtccccaccagtgttctccgggatggagcagcctcttccctcatctccctgctgccatccttgat

SEQ ID NO: 631 - DNA, 401 bp, Homo sapiens
actccctgaaggcggggctgatgtatcccaggcatcgcacctacagtgtctagggcccatgatgcttttaggggaagtgttttcatttcttttcaaagtagaagtgaaagaaaaaaagaaaagagaaaattgtaccctggataatatcggtctttgcagcaacaaagtcataaaatttaaacttttttttttttttttgaracagggctggagtgcagtggcgttaacacggctcactgcagccttggcctcccgggcccaagcgatcccaaccccaaccttctgagtagctgggactataggcaggcacaatcacatccagttaatttttaaatttttttgcagaggcgagttcttgctatgttgcccaggctcatctcaaattcctgggctccagcgat

SEQ ID NO: 632 - DNA, 401 bp, Homo sapiens
aagagttcaaggcttcattgagctatgattgtgccactgcactccagcctggatgacagagtgagactgcgtcattaaagaaacagaccaaccaaaaagacaagtgtaaatttcaattaattaaaattaatttgtaaaattcaatttctctgttgcactggccacatttgaagtattcaacagttacctgtggctggtggstaccaaattggacagtatagatatggaacatttcatcatcacagagagctctattggttctagtccagagtttcctggggtctgtattacatgcctctcactacacttcccttcctgaattgggactgtctgtttctatgtttgtctcattgattcattcattccagaaatatttattgaggacttcctttatgccaggg

SEQ ID NO: 633 - DNA, 401 bp, Homo sapiens
atggggaggggtcagggtgatggggaggggtcagggtgtggggaggggtcagggtgatggggaggggtcagggttacaagcattacaggtaaggggtgagggtgatgggggggtgtcagtgtgacggggaggggtcagggttacaggcaaggggtcagggtgacgggagagggcttagagtggcagggaactggttagagwgagggggaggggtgtccccgacagaggagggtgcagagtgacagacgcctcgtcagatttgcaagctgacaaaagacacttcagcaccatgtggaagaagaagggacaaaaatgggttagcttgacataagaagggtcagggtgacaggagagatgtcagactgacggaggaaccataacaatctgatggggcatcgcac

SEQ ID NO: 634 - DNA, 401 bp, Homo sapiens
agatagagatggagagacagagagacaatggcagagatgaggagacagagatagagagacaaggagagagctagggagacaaggagacacagagacagagatggagagacagagacaaagagacaaagacagatgggggagagagggaggtggggaaacagggagggatgggaagagaaagatagggagagtgagagaggsgaagagcgacagacagagatggggcctagaagggggagagacaaggactgagagatgagaggggaaatgagagggcgatgggaagcatgggaagcaggaagagcccagagaggaaggagggagggcagctctgactcagagagggcctgcgcagccccaggagggaaccccagaagtggggagtgcaactgagagggagc

SEQ ID NO: 635 - DNA, 401 bp, Homo sapiens
ccagagaaagccaaagccataccgaggggcggatggacagcagagccctaggaggagacagcccaagaggtgccctgacctagggtggatctgctgggctccgcgcccctgctgtccctacctcctctaggttatatgcaaaagtgcatctttggagagggttgaaatctcccagtttccaagtccttagatagtggcatmtttttttttttttccccgagacagaatcgcaccctgtcacccaggctggagtgcagtggcgcgatcttggctcactgcaacctctgcctcccaggttcaagcgattctcctgtcccatgctcccgagtagctggaataacaggcgtgcgccaccacacccagcaaatttttgtagttttagtagcgacggggtttcacct

SEQ ID NO: 636 - DNA, 401 bp, Homo sapiens
tataatagcaggggtggaaagtggagagagaaaaggacagagatgggatttgagggaaaggggataaggaggaaaggaggtctccaaagttgggagaaaggaggaaaccctaggatgtgtgtcgcccctccaggaccagcctaaagcacagaggcgtcaggagggttttggaaagagaaaggctccaggttggtgtgtgawccgcctcccccctggcagttcctccccacaccaggccgcctacctcagtggcaaagaaacctttgggtcggtgtttgcccagggacgcgaggccaccgggaccagggctttcgcttgcactgatggtctgttgagctgcggccaggatttcgtccagtttgtctaggggtagatgaatgaacagaggtgggaagacaatg

SEQ ID NO: 637 - DNA, 401 bp, Homo sapiens
atggtccagccctccttcccctctccccccggccccctggcctcagcagccccgcaggctcacccatctcctccagctctgaggtcatagatttggaacgcagggagatggttgggggcggcagccgcttggcctgctggggtgctgaaaaggtgatgaggggaggcatcagtatcatgggggagatgcccccaccaccckctaccaccacagggctcccagggctcagagaatagccattgccagcgtgcggagccctccctgcctcagtttctccccacaagggtcacggagaagcagtgctagggaagggaaggccttgggcagcagcaaccagccctggggctcccgcagggctgagcctggtggcagtggggtggctgcgagagtggccgcaggag

SEQ ID NO: 638 - DNA, 401 bp, Homo sapiens
cctattcacttccccctccctccttgtccccagtatttatttatttatttacttttatttttaaaagacagggtctctctctgtcacccaggttggggtgcagtggcccaatcatggctcactgcaccctcgacttcctgggctcaagcaattctcccacctcagcctccagaatagctgggacctcaggcacacagcacyggctaattttttttttaagagacaggatcttgctatgttgcccaggctggtctcaaactcttgagttcaagcaatccacctgcttcagcctcccaaagcactgggatcacaggcgtgagccgctgtgcctggccatcccccggtattgtgtatgtgttggggacagctctgtgtcactcttttgagatgcctcagaggcc

SEQ ID NO: 639 - DNA, 401 bp, Homo sapiens
gaaaggggccgagaaggccagaacagcaagtggacggggaaactggacacagaaattggctcagaggtcagagctgcctgtttctaaccatgcattctgtaatgtccccattcccaccttcttactcctcctcctgtatctcttccaggtctccaaggccccccagcagccagcagcacccttcctcactggaggcaacckcactttctttcactcatgccacaaacccctcccttcctccatctttctccctcccctctggaatctctcttgccccctcatcagcctcataccccatacataagccttccccaaatgtcagccaatatagtcaataaaactcccatatcaatatgccaagaaggagcacttgccattggctgggcagtatctgaagccct

SEQ ID NO: 640 - DNA, 401 bp, Homo sapiens
acaatgaggaacacttatcgaattattactgaatgccagccatgattccaagcactttgcatttatatatttccatttatgtattcagttagtcctcacaacaacccagggaagggttgactgttattcttcccattttataaatggggaaaccgaggcacagagaaataacttgctcaagatgacaccagctagaaaaawgtcaaagccaagatttgaattcaccagatatatatatatatatacacacacacaccagtggtgatctgaggcagggaggggggtccaaccacatccagacccaccctgttcctgcttctgcttctcaaccaccccctatcctacacatgctccattcccctctctgctccatgactccctcacccaatccccagctagga

SEQ ID NO: 641 - DNA, 401 bp, Homo sapiens
acaggatggacccttgaagtaggggcctgtgggggaggggtaggccaggcccaggagggtgggtgttgggaagcacagccgcagtccctgggacagacccactccagcacacaccacacacacacacacacacacacacacacacacacacacacacagtcccagacagcctcgctgtcgctccaagtcatacagagacaracgctgtcacacagggacacacagatggacacagttggggggtgggctgggagcccaccatctctccatttctctctctcacacacacacgcacacacacgcacgcggctgcaggcagtcacacacacgcacatcgacacagccacactgacacacgcaccgtcgcacagtgacacgcagcggaaaaagaggaggggagg

SEQ ID NO: 642 - DNA, 401 bp, Homo sapiens
ccagccccacagtcatcatagccacaaaggcaccagtatatacagagcagggcagaccctccagccccgggcataggtacaggtacatccaggccacacatgcacacagatgcgtgtcatgcacacctacagagacacacgttcacacaccaagaaacctcagctctggtcgtgcacacacgttcacacgccccccaggcrccccgtccttcccctcctgaccccatcccctccctgcccctcgcctctcacctccctgggccaatcccatccattaagcttccgaagcaagtcagccagcagccagggaggggaaaggtgtcagtgcacccatctcctcaccccaatcccacccagccctgactgcaccctccccacaccggtgcatgggacacacacat

SEQ ID NO: 643 - DNA, 401 bp, Homo sapiens
gccctgaccaccccgttgtctctggctaccccagagctgggcagtcttagtggctgccgaagatggtttggggttgggctgtactgtctgaagctaattctggcttatcccacccctggatcaaacttgagaaggaacagattagaaagggggcttagaggtgatggaaatatttaaaggtgtacattgtgaagggaactwggggtgaggggggttgcccgagggtgtaaggacatttaccttggggcatcaaagctgtcgtaggagcccacggtataatgccggaagagtctctttgccttgtcactgcggctttctgcagggtgacaacagacaaccttggactcttgttttggaaaccacaatcccagccctagagctcctggcccaagtcttcccat

SEQ ID NO: 644 - DNA, 401 bp, Homo sapiens
atcaaagctgtcgtaggagcccacggtataatgccggaagagtctctttgccttgtcactgcggctttctgcagggtgacaacagacaaccttggactcttgttttggaaaccacaatcccagccctagagctcctggcccaagtcttcccatctacctcccagtaccccacccccattaggaagggaaaggagtggacgwgggaatcctagggtccattgaacactgctgggggttccgcattaccttgcttgctctcctgagagcgattcgccacttcttccaggcagtcagaggggaaccagccaacacgacctttgacctggccttcccagaagcctccttccccgatgctaagtactggatggggaacggggacatagagacatttctgtgtttct

SEQ ID NO: 645 - DNA, 401 bp, Homo sapiens
attcccacagttaccagctcctggcccaaggccatccttaataaacatctgaggaaggggcgaggagcttgggggagacccatatcatatcactcttgcattctagtttctggatccaataaggatgacaggcattggggaagacgactgttccataacatggtcttcctaggttagaggcaggtccacaattggtccttstctcccctggtaactggtcacagctcagttgtgtaattccccttcaccctcctaggctctgtggacttagatcacggatggggcgacaagttctacctcaccctcccatcactgtccctgccacctcccaaagtctccttgccacttaatctttagtcctttgtccccagacccctgtctctgtctctctcggcctctcc

SEQ ID NO: 646 - DNA, 401 bp, Homo sapiens
ccacttaggaaaatgatggctttttttttttttttttttgagatggagtttcactcttgtcagccaggtgtgatcttggctcactgcaacctccgcctcccgggttcaagcaattttcctgcctcggcctctcaagtagctgggattacaagtgcgtgccaccacacctggctaattttttttttttttttgtatttttartagagatggggattcaccatgttggccaggctggtctcaaactcctgatctcaggtgatccaccctcctcggcctccgaaagtgctgggattacaggtgtgagccaccttgtccggccaaatgatggctcttatatggcacttgttatgtaggaaacattggtctaagtactttacatgatctgactcctccaatcctca

SEQ ID NO: 647 - DNA, 401 bp, Homo sapiens
tgactcctccaatcctcatcaaagtcccattttacagatgaagaaactgaggcccagagaaatcacttggctgaggtcaccctgctaggaagtggtggggtctggatttgaacccaggctgccaggctctcaacctctataccgctcagctcctgggcagcctagagtctggcaaagaattgttccaatgcctcgtattawcggccacacctgcggcacgtggtggtctctaaagcatccttctatccaagaacacgtcagcagccctgcaaagcctataagcaggggagactggctccactttccagatgaaggtggaaaaaccaaagggaggtgaaccctgataacagcaccaccattttgggaaccctcgccacggccaaacatcttatatctgccag

SEQ ID NO: 648 - DNA, 401 bp, Homo sapiens
actcccagcttcctcctgggtcaggtccaactcctctgcccgccgttgccagcctctcccccacttcctcgggacacgcctggctggccttcgcctccttctcaccctcctctagccaggtgggcttcctgaccccggaatgtcccccgccccttcctcatcctccctccagagtgacctctctgtccccctcaagtccckgccaccccccccatcctcttcctgttcagcttccctccacctcaccctgtcattctgtctgtccccaggggctggggcccatgtttctttggggtaccctctgcctggggcccctgtcctggagcagacatccagctaacatgtggtgcaccgggggtggagtcgggacagtagctcccaaggccttgctttaccccaag

SEQ ID NO: 649 - DNA, 401 bp, Homo sapiens
ccagcttcctcctgggtcaggtccaactcctctgcccgccgttgccagcctctcccccacttcctcgggacacgcctggctggccttcgcctccttctcaccctcctctagccaggtgggcttcctgaccccggaatgtcccccgccccttcctcatcctccctccagagtgacctctctgtccccctcaagtccctgccwccccccccatcctcttcctgttcagcttccctccacctcaccctgtcattctgtctgtccccaggggctggggcccatgtttctttggggtaccctctgcctggggcccctgtcctggagcagacatccagctaacatgtggtgcaccgggggtggagtcgggacagtagctcccaaggccttgctttaccccaaggtga

SEQ ID NO: 650 - DNA, 401 bp, Homo sapiens
cggggggcagatgttaggggagaaaaggcaagcagggggccatgcgggggcaagagatggtgggagagaggcatgagggagaaagacagagatgagaaagggaggggggcctggaattgctcccccaccaccatccagacatttttggtcctctagggttttctccatcctatcctggcccccaagtcaggtcccccaacycccccatcccaccatgaaacctcaaccgccccaggttctctgtgcagtccgagctccctgtcactcacccacatcctgttctcggtggtttcggatcagctcccccagctcaaaattcccagcaatcactgccacctggagggagggggtagaggcaggtcggccaggggggcccaggcagcagagagagggcagggaac

SEQ ID NO: 651 - DNA, 401 bp, Homo sapiens
tgagactctgtctcaaaaaaaaataataatttctgtattgattctatgttgaaatgataatgttttagatacactgttataagtaaaatatgttgttaaaataaatgtcacctaattattttccccttctctttaacatggccactaggatatttgagggcatttagggtatgtgctcatgctatgttccactggatgttrgggccggataatcctttggtgattcattggtttgtggagggtgttctgagccctgtgggatgttgagtagcatccccggtctctaccactagatgccagtagcactcccctccccagtcatgacaaccaaaaatgtctctagacattgccaagtgtcccctgggagacagaagctcccaggttgagaaccacaggtctag

SEQ ID NO: 652 - DNA, 401 bp, Homo sapiens
ggtatgtgctcatgctatgttccactggatgttggggccggataatcctttggtgattcattggtttgtggagggtgttctgagccctgtgggatgttgagtagcatccccggtctctaccactagatgccagtagcactcccctccccagtcatgacaaccaaaaatgtctctagacattgccaagtgtcccctgggagmcagaagctcccaggttgagaaccacaggtctagaccttacggaggcttaccatgttacgtgccaggcgcttggcactttagaggcagaagctcattgaattgcccaacaattccaggacatggaaattgtctctattttacagaggaggaaactaaggctttcaaaagagcccaaggtgggccaggggtggtggctcact

SEQ ID NO: 653 - DNA, 401 bp, Homo sapiens
tttgccaggccacagaaaatgcagctaaaattagctaaggacagacaggtggacaaactgacaggcatcctgccctcctggccccagcctggcccctcatcccagggccggggggtggggttggggagaagctgaggctgacactgagatgtagtaagggggagagggaggtgtggtgagcctggagcgggtggagctggygacttcactcaagtgtggctgacaacgcgtggcatgccgaatagtgctggggaatctgccgtcccaggggagcaaggggcactgagatgggggtgtcatgggcagagcatttggcctagggtgtccccaagggaaccacaagaggtggcttttggagactggggcatcttgagagggtgtgcgggtaggtggggaggtcg

SEQ ID NO: 654 - DNA, 401 bp, Homo sapiens
gagggccgaggacctcaccccccccgcgggccgggcctggccatccgcagagcgcccccccttccgccgcggccgccgcgcccctcctcgcccgcccccggctcggtccggccggctccgggcgccgcgtctccgcctgctcttcctcctcctcttcctcgggccgccgccgccgccgcccgctccgcgcctcctctgccrcccttctcgctctctcgctctcgctgtctctccctcaccctgtctctctccctccctctctccctccctcccctccctccgccctctcccctccctccgggagccggcggaggagacatcgcccctccccgcgcccccaaacctcgcccatcccccaccaccgccggctccgccccctacccttgccctcccccaccact

SEQ ID NO: 655 - DNA, 401 bp, Homo sapiens
accccggtatgcatgcccagatctctcaacaccaaaacggagttacagcactgtagagacgtgtgcacgccttcccatcacacccacccaccgtgctgtccattgcactggctcgcactcacccgtttacatgcccaacaccgtgccatccttcccctccaggatagttactgctgacattcctggagcactttctgtgtkccaggctcagggactttcgccatccccattttccagagggggaaaccaaggaacagagaggctaagtaacttgcccaaggtcatccagctgtaaggaggtggtaacaaggatcaggaacaggtaggctggctccaaagtcctcacttgacaactatggtgcactactgggcatatcccctcatagttgcatatccttatt

SEQ ID NO: 656 - DNA, 401 bp, Homo sapiens
ggtctcacctgtctagcatccctgggacaacagtaggagccgcttgagcttgtggcttctcctcagcaaatatgggaaacagagaaagatctggaatcagacagaggttggtgtcatttctgcctgttgtaacctgtcatctagggcaggtccctctcttcctggggctctcagtcaggttctgtacaaggttctcaccckccccacggaaggaggctgccaggaggagccagcaaagctattcaacttgcaggggctccagaatagcctctcctgtggcctctgtccactccctacccctgcaccttcagcaattcccacccagtggcgaccgtaagatgcccagacacagagcattatttctgctctttcactgcatacccaatgttagtccaaactgc

SEQ ID NO: 657 - DNA, 401 bp, Homo sapiens
ggccaggagtttgagaccagcctgggcaacatagcaagatctcatctctaaaaaaagaaagaaagaaacgaaaagaaaaatatgaacttgttaacaattctcattatctgagggaagaacgaggatggcaggggactctttaactaccaattttagtttgagctgcaatttccccctcatttagcattttcggttttattyaagaaaaaaaaaagacaaaaattagggttcaatatcataaaacatagagcaacccggagaaaagaattgcctattcacatctattcttttagctttcttacgttttaaccacttgagccgtaacggggtgtgaggggatttgcggtgatggaactgttctgtgttttggttacggggtggtcacgtgatgcgtgcatttg

SEQ ID NO: 658 - DNA, 401 bp, Homo sapiens
gagacttagtctctgcaaaaaagaaaaaaatgtaaaaattagcccagtgtggtggtgtgtgccttagttcccgctactcaggaggctgaggtgggaggatcgtttgagcctgagaggcataagctgcagtgagctgagactgtgccactgcattcccgcctgggtaacagagtgggaccttgcctcaaaaaaaaaaaaaargaaagggaaaaattaactgtcaaaacagcctcaggcagctccttcagaaggtgtccagaaaaaagcactgttatcctaggagacgacagctccaagggtgttattgcccctgaagatcttccagtgggataagacgtggagatggaagacagtgacgttgatgatcctgaccctgcataggcctaggctgatgtgtgtgt

SEQ ID NO: 659 - DNA, 401 bp, Homo sapiens
ccagcctggtcaatatggtgaaaccccgtctctactaaaaatataaaaattagctgggcatgtccccagggtgtgtgcctatagtcccaactactctactcaggaggctgaagcaggagaattgcttgaacccgagaggcagaggttgcagtgagccaagattgtaccactgcactccagcttaggcaacacagtgagatsctgtctcaaaaaaaaaaaaaaatgctggggcgctgtgtgcttggatgatagggctgtggagacaggacatagtggacagaggcaagaggctttttggaggcaaaatcgacagggctttgtgatgaattgggtgtgcgtgttgggggagtaggagacagaggtattcaaggattctacacctctatctcagacgagcatac

SEQ ID NO: 660 - DNA, 401 bp, Homo sapiens
ccaataaaatgtaagtggcagcaatcaatgtaacttccttgaagagccattaaatggaagaggcctgtcctccatttccccatctccccctttctattgtccagaatgcaggcataccagttagctcttgttgcgcaacaaacaccatcaaagtttagaggcttaaaaacataaccattttttttatagtgtcttgttctrttgcccaggttggagtgcagtggtgtgatcatagctcaccaccacctcaatctcccaggctcaagtagtcctctcacttcagcctcctgagtagctgggattacaggcataccccactgtgcccagctaatttttttttttttttcttgagacggaatctcactttgttgcccaggctggagtgcagtggcgccatctca

SEQ ID NO: 661 - DNA, 401 bp, Homo sapiens
gctgctggtatccagctgccttagtgaagtttccccgggatgccttacaggcatttcaaaggcgaagtgaccaaccccgagctcctggtcttaccccaaacctgctctcctgccgtctcccctatcccagtgcacagcaactccagcttccatcggttcaaaagtcctgcagtcaccccgacactgctctccctctcaccwcacacatccagcccagcagcaaatctagccagctccaccttgaccctggaatctgcttcccaccccctcctctgacatcaccctggtccccatccccatcactgcccacctgggttactgcagcagcttcctcccggcctcctgcttccatcccaccccctcagtctagtctcaaccttaaagtcacagtgattgttaag

SEQ ID NO: 662 - DNA, 401 bp, Homo sapiens
ctgctgccaattttttcaaatagtggagtaatttttaatattttgtccagattttaattttttttttgtctgtggcaccatttgtctgacaaagttactctgacattatccaaagttaaaagaacctccatctcattatttttactttatttttattgagacagggtctcgctttatcacccaggctgaagtgaaatggcrtgaccatggctcattgcagcctcgaattccagggctcaagtgaccctcgtgtctcagtctcccaatttgctgggactacaggcacgtgccaccatacctgactaattttttttttttttttttttgagacagagtcttgctctgttgcctgggctggaatgaagtggcacgatcttggctcactgcagcccctgcctccc

SEQ ID NO: 663 - DNA, 401 bp, Homo sapiens
ctacctattctgttgactagtggatctgattaaaaataaataaactggggaaagtattatttaaaaaaaaaaacttgaaagtgatgagaaatagtcctccctttcattccaaatcctccattgttactttttctcacgagaaaactgttaccagttttttgtgtatccaacccatgcataagcactttctttctttctttwtttttttttttttttttgagacagagtctttctctgttgcctaggctggagtgcagcagtgagatctcggctcaccgcagcctccgcctcccggggttcaagtgattctcctgcctcagcctcccgagtagctgggattacagacacccaccatcatgtccagctaacttttgtatttttgtagagacagggtttcacca

SEQ ID NO: 664 - DNA, 401 bp, Homo sapiens
attagcctaaaatgttggtgatccacaaaacttctattaattcgagataaaattatagtgcaactatgaatgtttattactgaaatttggggccaagcgcggtggctcacacctgtaatcccagcactttgagaggccgaggcgggcagatcactaagttaggagttcaagaccagcctggccaacataatgaaaccctgwctctacaaaaaatgcaaaacttagccaggcgtggtggcgggtgcctatagtcccagctacttgggaggctgaggtaggataattgcttgaacctgggaggtggaggttgcagtgggccgagatcgcgctagtgcactccagcctgagtttaacagagcgagactccatctcaaataaaatacaataaaataaaaagaaat

SEQ ID NO: 665 - DNA, 401 bp, Homo sapiens
tggtactgccttcaggatgggaggaaggagtagctttggggggttggttctaagaagcctgggggacatctggatgcgcaagtgtagtgctcaggagaaaggcctccagagagaagtgccgtctcagccctcgggtccacctgcagcatgacagcaccctgctggccctccagggggccctgggcctctatgatggacacwccccgccatatgctgcctgcctcggctttgagttccggaagcacctggggaatcccgccaaagatggagggtgagaatggtttggtgcccagggacatgggtgggacagaactctcaaagcaaggggtgatgtgtcaggcagagggcatggccaggtggggagctgcatgggatgatgctgggaggatgacaggtgggag

SEQ ID NO: 666 - DNA, 401 bp, Homo sapiens
acaggatacccagtacgatgctggttcctgcatgagcacaggtgttaggaggctgagaatcagatatgtaaggactccttcccatagtcatgtggctgcctggctctggctcattatttccccactttctttaacctcccagaccctgtacctttgctcctaagcatgaagatgggcttctgaccaagtcctgctagaggrcactgaaagggcaagccttgggctttaaccaggcaatggtaagtctgtaaccacaggttttgaggccaacagcagggatcttccctgcctccacctctggacttagggcagccatgccatatctctgagcctcagtttcctcatctgtagagtgggcacaatcataggacccatctccttgggccgtcatgaggagttac

SEQ ID NO: 667 - DNA, 401 bp, Homo sapiens
aaatttaaattgggctccagagaagagacattgagactgatacttccagcactgaggatctacctctaggttggtgagggcagcttcttgggcagtgagaagtggggtgggcagtgaggagcattcccagaagtgaggaccactctagggctgtgaggacaatcctagggcagtgagaaccctctgaggcagtgaggaccstcccagtcattgaggaccatccgagagagtgaggactatcccagggagtgacgaccatcccaggcagtgaggaccatctcaggcagggaggaccatcccaggcagggaggatcatcccaggcagtgaggaccatctcaggcagggaagaccatcccaggcagtgaggaccatcccaggcagtgaggaccatctcaggcag

SEQ ID NO: 668 - DNA, 401 bp, Homo sapiens
gaaaccatgtctctactaaaaatacaaaaattaggctggcgtggcagcagctgcgtgtagtatcagctaatcgggaggctgaggcaggagaattgcttgagcccaaaggcagaggttggagtgagccgagatcacaccactgcactccagcctgggcaacagagcaagactccatcttaaaaaaaaaaaaaaaaaaagaakaagaaagaaagaaatgattgaggtgattctgtggggtaaccttgaggatgagtgggatgacctttggggggtcctctgtggggtgaccttggggggtgtgtgggattatgagagtgtctgagaattgagttttaggttaagtaggggtgatttgggacaactctaagggtgtgactgggggatgactttaggggtccctg

SEQ ID NO: 669 - DNA, 401 bp, Homo sapiens
ctccgaggggaccggtggcaaactccagccctcgctctagggcccgccctttgcggctttgtccctgcccactgggtcctcacagtacctccaccccagaggcccagcatcccctcccgttctgcagacacttgtccagcgtcccgctgagctcggcagtgtggctagtttgtgtggccctgcccctggtgcccagccagrgtggtctgcatggaacttctttcccggagttccggcctcagctccgatgtccacccttgtccccaggtggacacagcaccgcccagcatttggtgcaactccacccttctgcagaacgggcccctccctgggaaggcatggcttgcaataacatccctctgtaccttttctggcttccccgccacaataaaacctctcag

SEQ ID NO: 670 - DNA, 401 bp, Homo sapiens
ggattggagaagtggtgagaacaggcgccaagcagctgtgggaatgcaatgcaggacccacttgacgtgaggttgtagaatcacatactgacttgggggaggccagggcagaagcgctggctgagaaggcattcaggatccttggggtggggttccagaaaaccagagctgtgtaggaacaattcaggcagagaagggctkgggcctggggcgggatggaacttccccaaggaaaggagcatgtggtgggagggtgtggtgtttttcctgggagagctgggggtgctgggtgggaagggacatttgcagacccccccccataccagatgggggatcccacatgccagtttgggaagcagggttttctggtgaaatttgggggaccctggaaggactgggga

SEQ ID NO: 671 - DNA, 401 bp, Homo sapiens
cacccctgccctgctggcacccagatgttggccaaggcccttcccctgttgcctgggggctggacaattgccccccccccctccgatccccagagctgtagacggggagggagaaccagcacctgttctgcaggccctggatcacgctggacgcattcctggggttgaagccaggtggggcagggcctgtggctctggggmtttgctctggggagcagccagtgaggcaaggggggactgaggggaagcagaaacaggaagacggaggaatcagtgacccctggtacaaagccccatatacctcagtctcaattaaggccctgcacccccagacctggtgccctcaaacaccagcagttagggctctgattccctgcaccactcggagccaagatactagc

SEQ ID NO: 672 - DNA, 401 bp, Homo sapiens
ggggaggctggaggcgtgggggtctcaggggcccccaggagtgagggtcatttgtggcagaacgttaatgaaagtccaccccccaatttctccagaagagaggcatcaggttacaattaatttcttggtgtgtggcagctgagttcaatctggagtttgcctgacacccaaggtgaccttgatgttgtggccgtccctctragccacagccccttcccttgtcaaatgaggtcggtggtgcccaccacacatagacgttgtatgtaagctctaagcaccgtgcctggcttgcacacctgaaggggcactacctgggccaggcttatgcttctgcctcacacacaaccacatttaatcccctcaaagaccctgtaagagacagggagtggcaggtgtgggac

SEQ ID NO: 673 - DNA, 401 bp, Homo sapiens
ctcacctgtggatgccagcaggaaggagagagtgaggagaagccacattgtggaggagggaccagggttcggctgcagtctggggagggagtgagagaatccaggtgaggcaggacccttctgcctccaccaggatccctccagacacagacttatcccatccactgttaataaatttcttctgaccaccggtatcccctycccccaaccccactctgcgcttccttctgggtcccccatctctggaaggtttactcttcctctaggcactcggacagaccctgccccacctcctccacctgtcagcctccagcaccatgctaccagcccctggatctctctccatcatcaccaaggacccctgtcccaacttttttttttttttgagacggagtctcact

SEQ ID NO: 674 - DNA, 401 bp, Homo sapiens
taataaatttcttctgaccaccggtatccccttcccccaaccccactctgcgcttccttctgggtcccccatctctggaaggtttactcttcctctaggcactcggacagaccctgccccacctcctccacctgtcagcctccagcaccatgctaccagcccctggatctctctccatcatcaccaaggacccctgtcccracttttttttttttttgagacggagtctcactctgtcacccaggctggagtgcagtggcacaatgtcggctcactgcaagctccacctcccaggttcacaccattctcccgcctcagcctcccgagtagctgggactacaggcgcccaccaccgtgcccggctaattttttggaaattttttttttttaagtagagacag

SEQ ID NO: 675 - DNA, 401 bp, Homo sapiens
aataaatttcttctgaccaccggtatccccttcccccaaccccactctgcgcttccttctgggtcccccatctctggaaggtttactcttcctctaggcactcggacagaccctgccccacctcctccacctgtcagcctccagcaccatgctaccagcccctggatctctctccatcatcaccaaggacccctgtcccarcttttttttttttttgagacggagtctcactctgtcacccaggctggagtgcagtggcacaatgtcggctcactgcaagctccacctcccaggttcacaccattctcccgcctcagcctcccgagtagctgggactacaggcgcccaccaccgtgcccggctaattttttggaaattttttttttttaagtagagacagg

SEQ ID NO: 676 - DNA, 401 bp, Homo sapiens
gacttcatgatccacccacctcggcctcccaaagtgctgggattacaggcatgagccaccgcgcctggtctatctcaaccctcttatctggccttttgggtccccctccagtccagtcccctccaccagcttgccctccagccatgaccccaaagggtctttctacatctggagctggctgtgcccctcccctgctcaccrccctccatggatccttggcaccaccaggataaagtccagctcttcaaattcactttctttctttttttttcttttttttttttttttttgagacggagtctggctctgtagcccaggttggagtggagtggcgtgatctccgctcactgtaacctctgccttctgggttcaagagattctcctgcctcagcctcccaagc

SEQ ID NO: 677 - DNA, 401 bp, Homo sapiens
ggagattaatgacctgcagtctggaatatggatttcaaagtctgagttcttgggctgggggtctggaatctggatcctggagtcgggcctttgagtgacagatcgtgaaccttgattctggatcccggggcattcgaatctagaacccacaataggagcctggaatctggtgtttggaatctggcaatcgaggctggaaamctttgcacctgctatgagaagcctggggcctagactacaaatggtagatttgggattttgaattgtggggtctggattctttttttttttttttctgagatgaagtttcgctcttgtcacccaggctggagtgcaatagcgtgatctcagatcactgcaacctccacttcctgggctcaatagattctcctgcctcagcc

SEQ ID NO: 678 - DNA, 401 bp, Homo sapiens
acagatcgtgaaccttgattctggatcccggggcattcgaatctagaacccacaataggagcctggaatctggtgtttggaatctggcaatcgaggctggaaacctttgcacctgctatgagaagcctggggcctagactacaaatggtagatttgggattttgaattgtggggtctggattctttttttttttttttctkagatgaagtttcgctcttgtcacccaggctggagtgcaatagcgtgatctcagatcactgcaacctccacttcctgggctcaatagattctcctgcctcagcctcctgagtagctgggattacaggcgaccaccaccacgcccagctaatttttatatttttagtagagacagggtttcaccatgttggccaggctggtg

SEQ ID NO: 679 - DNA, 401 bp, Homo sapiens
ggctgaggcttgcaagaccggttaacacggcacgtgattcagatgtagccgctggagccaggtggcccgggtttgagttctgcctctgtcccataatactgtgcaccctcaggcaagtcacttagcctctctgtgcctcagctgcctcaactataaagtgggggtaataattgctcctccctcttctgtgggagtgttttsaggtgttcattcttgaaatctgtgcccggcactgagtgagtgctgggccattatcagtctgtcctattacccttgggaactgaaagctggggagggatggttatgtggggtctttctcagatgacacagttgggtagcagcagatttggcactggaagaagccactgtgctttttgttttgttttgttttctgttttttg

SEQ ID NO: 680 - DNA, 401 bp, Homo sapiens
ctgtgggagtgttttgaggtgttcattcttgaaatctgtgcccggcactgagtgagtgctgggccattatcagtctgtcctattacccttgggaactgaaagctggggagggatggttatgtggggtctttctcagatgacacagttgggtagcagcagatttggcactggaagaagccactgtgctttttgttttgtttkgttttctgttttttgtttttttttttttttgagacggagtctcgctcttgttgtccaggctggagtgcaatggcatgatcttggctcactgcaacctccgcctcctgggttcaagcgattctcctgcctcagcctcctgagtagctgggattacaggcacccaccagcacagccggctaatttttgtatttttagtagag

SEQ ID NO: 681 - DNA, 401 bp, Homo sapiens
tgtgggagtgttttgaggtgttcattcttgaaatctgtgcccggcactgagtgagtgctgggccattatcagtctgtcctattacccttgggaactgaaagctggggagggatggttatgtggggtctttctcagatgacacagttgggtagcagcagatttggcactggaagaagccactgtgctttttgttttgttttkttttctgttttttgtttttttttttttttgagacggagtctcgctcttgttgtccaggctggagtgcaatggcatgatcttggctcactgcaacctccgcctcctgggttcaagcgattctcctgcctcagcctcctgagtagctgggattacaggcacccaccagcacagccggctaatttttgtatttttagtagagt

SEQ ID NO: 682 - DNA, 401 bp, Homo sapiens
agccactgtgctttttgttttgttttgttttctgttttttgtttttttttttttttgagacggagtctcgctcttgttgtccaggctggagtgcaatggcatgatcttggctcactgcaacctccgcctcctgggttcaagcgattctcctgcctcagcctcctgagtagctgggattacaggcacccaccagcacagccrgctaatttttgtatttttagtagagttggggtttcacagtgttggccaggctggtcttgaactcctgacctcaagtgatccacctgccttggcctcccaaagtgttaggattacaggcgtgagccaccgcgcccagccagaagccactgttttgatctcccagatgtggtatctttttctgaatccctgtgaccccttta

SEQ ID NO: 683 - DNA, 401 bp, Homo sapiens
agactttatttcctgggctgtgctgggggaagctggaggggaggaggcagaaaaacccacatcttctcttcttttgtcctgggagggccttaccgataggtggcatcaattgtgcgtttgttcatagcagttttactcctgggtctgagggaggaggggctgggggcctggactcctgggtctgaggagggagaggctggwgcctggactcctgggtctgagaaaggagaggctgggggccgggactcctgggactgagggacgggtggctggtgtccaggactcctgggtctcaaggaagaagggctggggggtctgagggaggagggggctggggactcagactcttgggtccaagggaggagcggctggggtctggactcctgggtctgagggaggtg

SEQ ID NO: 684 - DNA, 401 bp, Homo sapiens
tactatctcagggcagaccctctctcctctctcttcctccatctctgcaccccacaaataaagtggcaacccccccgatgtgtgtctttgcagaacctgaatgtgatgagattgccagaaaatgccagtgtgcccctggatgggtggaggagggtggggcgggtggaggggacaactttgccccattggggaaccactggkctatacctgtcactgaagcttcctttctcccctgcacctgactccacacctcaggacgcaggtcaggagggctgggggatgctctagccttggccaggtgggcctggagatgattgttcactgcaggagcattcacctttttagatgggtgcttcctgtctagagaaggagacacggggagctggaaagagaagcctgca

SEQ ID NO: 685 - DNA, 401 bp, Homo sapiens
ctgtaatcccagctactcaagaggctgaggcaggagaatcgcttgaacccatggtggggtggaggttgcagtgagccgagatcgcaccactgtactccagcctgggtgacagagtgagactctgtctcaaaaacaaacaaacaagcaaacaaaaaaaccaggtaaactgaggctgggttttgtatcccagaccaaatgccygcactttgaatgcctggttgggaagcctgagattatcctgtagggaagcatcgagagcatgaactgaatggtgttaggagacaattatccatggctctcttacctttctgcctgtctcctgaaaaaggtattagccacttttgagttccagattgtctttttatggatgtttctctagagcatcagagcatgtttgttca

SEQ ID NO: 686 - DNA, 401 bp, Homo sapiens
tacaggcgtgaacgactacgcccgaccagtggtcatctttttgcaatgaatttcccaggcgttctttgagcttctcatgtttggatatctagatctctagcaaggccaaggaagattattcccctcaaatatgttttccaaacttttagatttatcttcttccttgggaacatgaattattcttaggttggttgtttaacrtaatcccaaacttcttggaggtcttgttcattttttaaaaatacttttttctttgtctttgttggattgggttaatccaaaagtcttgtctttgggctcttaagttctttcttctacttgtttgattctattgctgagactttccagtgtattttgcatttctctaagtgtgtccttcatttccagaaattgtaattgtt

SEQ ID NO: 687 - DNA, 401 bp, Homo sapiens
caggctacaagcctccctgctgagaaagcaagcagggctttcaggtttcgtgcctccccacctgctgcagcttctgtgctcatgtctgcactcacagttcaccccctcccctgagttctgtccaggaaacttcgtgttcagttgaaattgttgcaaagttcagctggaagcttccttctccctgtagtctttccccaattscgttggcagccctccccaaggacccctgtgagatgaagtcagaaatggcttccctggggaccaacagtaccctcagggctcttcctgctgctttctctaccccccgtatttcattcagctctctaagttcatctcggctccaggtaaggtcaaatccttctcccatgatctggaccttcagattccccagtgaggatgtg

SEQ ID NO: 688 - DNA, 401 bp, Homo sapiens
gtccttctgtgtccctctctgtccctgggggagggcctctctgaccatctcaggctgttatagtggagattctggaactctggagtggaaggattcctgagacctttctgggggtatgcgaggtcaaacactagtttcataataatctgtacacattatttacctttctcactcttttttttttttttttttttttttttstgagatggagtttcactcttgttgcccaggctggagtgcagtggcacaatctcagctcactgcaacctctgcctcccgggttcaagtgattctcctgcctcagcctcccaagtagctgggattacaggcgcccgccaccacgcctggctaatttttgtatttttagtagagacggagtttcaccatgttgtccaggctgg

SEQ ID NO: 689 - DNA, 401 bp, Homo sapiens
tctcacaagcacaaggtggaattttccagagcctacatgctgtgatattgtagcaaattaaaccctgaagcagatatgagagtccagctgccttctattaagccagacattaaagagacttgcaaaagtgcataacattttttctcactagtttttttcttaaaatatagctatttttcattaaaatacattatgttaacrtatttattattgttatttaaaaataaattaataactgaatattttaaagatttctcagttttaatatctactgtggtatcaatagatatagttaatataaacaaaaacccatgaaaggccttcagtaattcttttgttttgtttttttttttcgatggagttttgctcttgttacccagggtagagtgcagtggcacaat

SEQ ID NO: 690 - DNA, 401 bp, Homo sapiens
gggcagaggctggaaccattaggtccagggtttggggtgatagtaatgggatctcttgattcctcaagagtctgaggatcgagggttgcccattcttccatcttgccacctaatccttactccacttgagggtatcaccagcccttctagctccatgaaggtgcccctgggcaagcacaatctgagcatgaaagatgcccyagaggccttgggtgtcatccactcatcatccagcatccacactctgagggtgtggccagcaccatgacgtcatgttgctgtgactatccctgcagcgtgcctctccagccacctgccaaccgtagagctgccgacatcctcctctggtgggagtggcctgcatggtgccaggctgaggcctagtgtcagacagggagcct

SEQ ID NO: 691 - DNA, 401 bp, Homo sapiens
gggggaaggaaaacagggtatgggggaaaggaccctggggagcgaagtggaggatacaaccttgggcctgcaggccaggctacctacccacttggaaacccacgccaaagccgcatctacagctgagccactctgaggcctcccctccccagcggtccccactcagctccaaagtctctctcccttttctctcccacactytatcatcccccggattcctctctacttggttctcattcttcctttgacttcctgcttccctttctcattcatctgtttctcactttctgcctggttttgttcttctctctctctttctctggcccatgtctgtttctctatgtttctgtcttttctttctcatcctgtgtattttcggctcaccttgtttgtcactgttc

SEQ ID NO: 692 - DNA, 401 bp, Homo sapiens
ggggaaaggaccctggggagcgaagtggaggatacaaccttgggcctgcaggccaggctacctacccacttggaaacccacgccaaagccgcatctacagctgagccactctgaggcctcccctccccagcggtccccactcagctccaaagtctctctcccttttctctcccacactctatcatcccccggattcctctytacttggttctcattcttcctttgacttcctgcttccctttctcattcatctgtttctcactttctgcctggttttgttcttctctctctctttctctggcccatgtctgtttctctatgtttctgtcttttctttctcatcctgtgtattttcggctcaccttgtttgtcactgttctcccctctgccctttcattctc

SEQ ID NO: 693 - DNA, 401 bp, Homo sapiens
cacctccccgtgtctcatctcattccctccttccctcttctttgactccctcaaggcaataggttattcttacagcacaactcatctgttcctgcgttcagcacacggttactaggcacctgctatgcacccagcactgccctagagcctgggacatagcagtgaacagacagagagcagcccctcccttctgtagccccsaagccagtgaggggcacaggcaggaacagggaccacaacacagaaaagctggagggtgtcaggaggtgatcaggctctcggggagggagaaggggtggggagtgtgactgggaggagacatcctgcagaaggcgggagtgagcaaacacctgccgcaggggaggggagggccctgcggcacctgggggagcagagggaac

SEQ ID NO: 694 - DNA, 401 bp, Homo sapiens
acactgcttttcctctgaggagtcaggaactgtggatggtgctggacagaagcaggacagggcctggctcaggtgtccagaggctgccgctggcctccctatgggatcagactgcagggagggagggcagcagggatgtggagggagtgatgatggggctgacctgggggtggctccaggcattgtccccacctgggcccktacccagcctccctcacaggctcctggccctcagtctctcccctccactccattctccacctacccacagtgggtcattctgatcaccgaactgaccatgccagccctgccgatggtcctccatggctccctagtgccctggagaggaggtgtctagtcagagagtagtcctggaaggtggcctctgtgaggagccacgg

SEQ ID NO: 695 - DNA, 401 bp, Homo sapiens
gggaaagaatgcagaacctagaatataaattcatcccaacagtttggtagtgtgcagctgtagccttttctagataatacactattgtcatacatcgcttaagcgagtgtaaaatggtctcctcactttatttatttatatatttatttagttttgagatggagcctcgctctgtctcctaggctggagtgcaatagtgcrataccactcactgcaacctctgcctcctctgttcaagtgattttcttacctcagcctcccgagtagctgggattacaggtgcgtgccaccacacccggctaatttttgtattttttgtagagacggggttttgccatgttggccaggctggtcttgaactcctgacatcaggtgatccacctgccttggcctcctaaagt

SEQ ID NO: 696 - DNA, 401 bp, Homo sapiens
gctaatttttgtattttttgtagagacggggttttgccatgttggccaggctggtcttgaactcctgacatcaggtgatccacctgccttggcctcctaaagtgctgggattacaggcatgagccaccgtgcccaatcactttatttattttttatttttatttttaaatttcagcttctatttgaaatacagggggcacwtatataggattgttacatgggtatattgaactcaggtagtgatcatactacccaacaggtaggttttcaacccactccccctcttttcctccccattctagtagtgtgcagtgtctattgttctcatgtttatgtctatgtgtgctccaggtttagctcccacctgtaagtgagaacgtgtggtatttgattttctgtcc

SEQ ID NO: 697 - DNA, 401 bp, Homo sapiens
cctgccttggcctcctaaagtgctgggattacaggcatgagccaccgtgcccaatcactttatttattttttatttttatttttaaatttcagcttctatttgaaatacagggggcacatatataggattgttacatgggtatattgaactcaggtagtgatcatactacccaacaggtaggttttcaacccactcccccwcttttcctccccattctagtagtgtgcagtgtctattgttctcatgtttatgtctatgtgtgctccaggtttagctcccacctgtaagtgagaacgtgtggtatttgattttctgtccctgtgttaattcacttaggattatggcttccagctccattcatattgctgtaaaggatatgattcatttttcatggccatgc

SEQ ID NO: 698 - DNA, 401 bp, Homo sapiens
taggttttcaacccactccccctcttttcctccccattctagtagtgtgcagtgtctattgttctcatgtttatgtctatgtgtgctccaggtttagctcccacctgtaagtgagaacgtgtggtatttgattttctgtccctgtgttaattcacttaggattatggcttccagctccattcatattgctgtaaaggataygattcatttttcatggccatgcagtattccatattgcgtatagatcacattttctttcttttttttttttgagacggagtcttgctttgctgcctaggctggagtgcagtagcacgatctcggctcactgcaagcttcacctccggggttcacgtcattcttctgtctcagcttcccaagtagctgggactacaggcgcc

SEQ ID NO: 699 - DNA, 401 bp, Homo sapiens
ccagaatctacaaggaacttaagcaaatttttactttttaataatagccactctgactggcgtgaaatggtatctcattgtggttttcatttgaatttctctgatgatcagtgacaatgagcattttttcatatttgttggctgcttgtacgtcttttgagaagtgtctcttcatgccttttggccactttaatgggattmttttttgctttttagtttaagttccttatagattctggatattagacttcttattggatgcatagtttgtgaatactctcttccattctgtaggttgtctgtttactctattgatggcttcttttgctgtgccgaagcatcttagtttaattagaaaccacctgccaatttttgtttttgttgcaattgcttttggggac

SEQ ID NO: 700 - DNA, 401 bp, Homo sapiens
ctcttggtaagaacagggaaaatagagaaaaatttagattgcatctgacctttttttctgaatttttatatgtgcctacaatttgagctaaatcctgaattattttctggttgcaaaaactctctaaagaagaacttggttttcattgtcttcgtgacacatttatctggctctttactagaacagttttcttgtttttgstgttctagcttgtgtgccttacagttctactcttcaaattattgttatgtgtatctcatagttttccttcttttgagaaaactgaagccatggtattctgaggactagagatgactcaacacagctggtgaatctcctcatatgcaatccactgggcttgatctgcttcagattgctgatgcactgctgctaaagctata

SEQ ID NO: 701 - DNA, 401 bp, Homo sapiens
ggactaatacaaggatctccaaaattccaagtttatgtattctttcttgccaaatagcaggtatttaccataaatcctgtccttaggtcaaacaaccttgatggcatcgtacttcaattgtcttacacattccttctgaatgactcctcccctatggcatataagccctgggtcttgggggataatggcagaggggtccamcatcttgtctggctgccacctgagacacagacatggcttctgttggtaagtctctattaaatgtttctttctaagaaactggatttgtcagcttgtttctttggactctcagcttcctcagactttggggtaggttgcacaaccctgcccaccatgaaacaaatgtttaatatgataaatatggatagatataatccaca

SEQ ID NO: 702 - DNA, 401 bp, Homo sapiens
ggggattgaattggggatgaagtaggtttagttttggagatagaatacatggagctggctattgcatgcgaggatgtgcattagtttggtttgatctttaaatgaaggaggctattagggttgtcttgaattagattaagttgtgttgggttgatgggttgggcttgtgggtgatgtggttggattgggctgtgttaaatwggtttgggtcaggttttggttgaggttatcatggggatgaggatatgcttgggacactggattcaggtggttctcattcaagctgaggcaaatttcctttcagacggtcattccagggaacgagtggttgtgtgggggaaatcaggccactggctgtgaatatccctctatcctggtcttgaattgtgattatctatgtc

SEQ ID NO: 703 - DNA, 401 bp, Homo sapiens
ttggggatgaagtaggtttagttttggagatagaatacatggagctggctattgcatgcgaggatgtgcattagtttggtttgatctttaaatgaaggaggctattagggttgtcttgaattagattaagttgtgttgggttgatgggttgggcttgtgggtgatgtggttggattgggctgtgttaaattggtttgggtsaggttttggttgaggttatcatggggatgaggatatgcttgggacactggattcaggtggttctcattcaagctgaggcaaatttcctttcagacggtcattccagggaacgagtggttgtgtgggggaaatcaggccactggctgtgaatatccctctatcctggtcttgaattgtgattatctatgtccacttctgtc

SEQ ID NO: 704 - DNA, 401 bp, Homo sapiens
gaggatgtgcattagtttggtttgatctttaaatgaaggaggctattagggttgtcttgaattagattaagttgtgttgggttgatgggttgggcttgtgggtgatgtggttggattgggctgtgttaaattggtttgggtcaggttttggttgaggttatcatggggatgaggatatgcttgggacactggattcaggtkgttctcattcaagctgaggcaaatttcctttcagacggtcattccagggaacgagtggttgtgtgggggaaatcaggccactggctgtgaatatccctctatcctggtcttgaattgtgattatctatgtccacttctgtctccttcactgtacttggaattgatctggtcattcagctggaaatgggggaagattttgt

SEQ ID NO: 705 - DNA, 401 bp, Homo sapiens
ttcttgagacacagctgggtctggatcagcgtaagctgctctggttttattgaacagatgaaatcacattttttttttcaaaatcaaagaaatcttatagagttaacagtggactcttataataagagttaacaccaggactcttattcttgattcttttctgagacaccaaaatgagatttctcaatgccaccctaattytttttttttttttttttttttttgagacacagtctgggtcttttgctctgtcactcaggctggagcgcagtggtgtgatcatagctcactgaacccttgacctcctggacttaagggatcctcctgcttcagcctcctgagtagatggggctacaggtgcttgccaccacacctggctaattaaattttttttttttttt

SEQ ID NO: 706 - DNA, 401 bp, Homo sapiens
ttggaaaaagtaaaaaaaatggattaatagaattgagtagaggtgaagaacgcaatagtgacattttagactagattgaagaaaaatgtcaaagaaatactgaatgatgtgagtgctggcagtgcagatactgaacactaaagactcaggaagagagaaaaataggaaaggaggaaacacatgactaaacactgaaggcasgttttttttttttttttttttttcgcagagtttcactcttatcgcccaggctggagtgcaatggctcagtctcggctcactgcaacccctgcctcccagttcaagtgattctcctgcttcagcctccccagtagctgggattacaggtgcacaccaccacacctggctaattttgcatttttaatagagacggggcttct

SEQ ID NO: 707 - DNA, 401 bp, Homo sapiens
tggaaaaagtaaaaaaaatggattaatagaattgagtagaggtgaagaacgcaatagtgacattttagactagattgaagaaaaatgtcaaagaaatactgaatgatgtgagtgctggcagtgcagatactgaacactaaagactcaggaagagagaaaaataggaaaggaggaaacacatgactaaacactgaaggcagsttttttttttttttttttttttcgcagagtttcactcttatcgcccaggctggagtgcaatggctcagtctcggctcactgcaacccctgcctcccagttcaagtgattctcctgcttcagcctccccagtagctgggattacaggtgcacaccaccacacctggctaattttgcatttttaatagagacggggcttctc

SEQ ID NO: 708 - DNA, 401 bp, Homo sapiens
gcagttcgttctctactgacaaggctttattatccatgaatatcctcttggtgtctcagtgatgatgcatccactgtcatttctgatcttactaacatgtatcttctgtttttttttttgtctttgttatcctggctacagaattatcaattttattgatcttttaaaagaaacagttgttttagttgattttctctatasgttgtttttaatttcgccaatttctgccataatttttattatttattttcttaggtttgcattaggcttaaatttctctctgagtccatgggaggttggactggaagctgggattcagagtgggagtctgggagtgggtccagatagagggtgtggctggaggtgagttctttgggactggagaagatctaggggctagg

SEQ ID NO: 709 - DNA, 401 bp, Homo sapiens
catggagaattttctctcagaatcataatttaaattctctaaccttccctgcaaaacaaacttatactttcttccttataccccttgtctgccacatctgttaccctagcctcaccagtgtgccaggcccagttcccaaagatggcaggaaaaatatggggatctgatctggatgtgccctattgttgctgtctgtttatstgtggatttgactttgaggggacatatccacgtttcccagtgtagaggctcagggtcttctgactgtgaaaagtccttgtttggtacttgtgcgtagtgcagttctgtgtgcgtgtgtgtgtgttgtgtgtgtgtgtatgtgcatgcacatgtgtatgtggtgggtgtgcaaggccctgctgtccactacctgcccacac

SEQ ID NO: 710 - DNA, 401 bp, Homo sapiens
atgcaccatggtttatctgtccactcgccagtttatggaacgtctgtcactgctttaacactgcaaacttccctctccccaatcttccttattatttaacttattatatatgatttgtactaatttacaatattatgttaacacatataaatgtatatctaatttgttatttattttattggatgtattttctattgtttktatctttcagctacaacaagaacaaggatctttgccagttttgttcactggtttatccaaagcccctaaaaccctgcctagcacatagtaggtacctgataagtacttactgaataaatgagaatgaatggatggccacaaactctttctatcactgtatttctcagttgagcttctcggtcagggtactgcctctctgg

SEQ ID NO: 711 - DNA, 401 bp, Homo sapiens
aggaaaacagagatgtcaattacaatgttattattaccagagcaagaaatcatcaaataagacaacccgtcagaattccattttatttggggatcattttcataagacatggaataatttctaacatctttgtagtctcataagaccatttggtttaaattattcttccaagagcagattcgtattaccaaagactagaamaagtcagcaaccgtcatgaatattcatacaaaactgtatattgcagaggagaacataaatgatattcttataaacagtctcagttttgcacaacatcaaatcaaattccttgaaccattttataacacagaacaatatcttttgatttattgctgcaggggcaggaaacgtgtccttcaactctttctgagagtcatgta

SEQ ID NO: 712 - DNA, 401 bp, Homo sapiens
agcgacagcgttgaatgaagtgtgaactgctccacccttttgagaagtcatcagaaagagtctgtgaagactgaaaatgcgcgtgccctttgactctcagcttccaaaataaaacgaaccgcactgcacagcactagctccaagacgcttcttgaaaccctgtttgtgaagccagccctggccctgttgggtggggctccrggtcaaggcacccccagtgctcccatttacttagcaccttgactcaggaatgggttggactctcagagcttacaatgtctatgaaacactattagagatcataagaaatacattcagactcacaactacccacatgttcatggagagatgtagaaataaataagcggatcttactcttgtgtgttagtttagatgttagt

SEQ ID NO: 713 - DNA, 401 bp, Homo sapiens
ctatacatctacatctaccttggtaaggagtttgttcaaatcttttgcccattttttatatgtatgtatgtatttttatttatttttgtagagatgctcaggctggtcttgaactcctggcctcaagcaatcctcttgcttggcctcccaaagcactgagattataggtgcttcccattgtgtccagccctcttgcccatytgaaaaactgagttttctgtttgtttatgtttttgtttgttgtttttttacaattgcactttgaaagctctttgtgtctttgtgtattctggacatcagtctaagacttgaaaatattttgtttgcctgtggcttgcacaggaatgaacacctggctttattgtgcattgctttactgcacttcacagatactgtgtttt

SEQ ID NO: 714 - DNA, 401 bp, Homo sapiens
aggtggatggatgaaaagagagagagagagagagataaaggagcatgggcaaacattcactgtaaaatcccagtggtggatataggtgtgtttgtgttcattgcacaatcctttccactcacctataggtttgaaaatttcaacaataaaggatggaagaaaaccaaataactaaagaagaaaattatgcttagacatagwcaatcagaagtcatgatacatcctgggtgacaagtgactgatgagagggacagaccaagcccttgggctgcagggagtcagaggggctggtcatcagggaaggcttccaggatgaggtgccctatgaacagggctgtgaaggacaggtgggctcggcccttcaaggtgaggcagggcttgcagggtgcctacgccccacc

SEQ ID NO: 715 - DNA, 401 bp, Homo sapiens
actgaggcagccctcacactggggagcagctgcgagagtgtctcacacccaccagacacaaagcttcaggaatcggttttgtcacctccacacaagtcagatatgaagcgtttgcggggtacgtggcttgggttttgtttttagtgagaggtgatgcacaggctggtagttaggagcctgcaagcctgcaggctgggctcrtgtccaggctcctgccccaagtccacatgactttgggcaagtcacttcaccagtctgaacctcagtttcttcatctgtaaaatggaggtggctatacttctcacatggagcggtcgtaagaattagatgaaggaatatatttagaggatttggcacaatacctcaccacagaaaaggttcaataaatgtgaaccgttatt

SEQ ID NO: 716 - DNA, 401 bp, Homo sapiens
cttgagcatggttttaactcctagggggctttggtaatgtctggtagggtcagcttcatgagtgtgcttggggtttaatgcactgtagttgccatcttgaaattcttaatagttttatctttgactttgtgttgtgtagatgaagctgatgggacaatagagcgtatgccaggggcttagtctcagttcatgtggtccccscttctgctgcctccttgcctttctggcatgggtttcaggtcccagctctccatccactgatgcccctggcctggccaggcctctctcttcctactcccactcagcaaccaccgctgccctctctgtccttagggggtcttcatgtgggcatggagaggttgagatagtgtgcttttgcggcacgacatctcagggtggag

SEQ ID NO: 717 - DNA, 401 bp, Homo sapiens
agaagttattatctgggaaacctggacattgtctgaggacctggttgcctgtgacaacatacatttgtcgggagggattgccagggggtcagatatcagcccataaagttgctaatataactgatgcagcttgatgagaggaaaagcatcaatcaaagtcagaaagtctgtgtaggtgaggtgggggtgggagtgccggcyctaaagccttgattctgaaaacagaaggtctccacggaatttttctgtaggaaaaatgggaggccaagtaaaacctggtgctgggaacaggtgatgagcactagacttgacctctctaaatgaaggatgtagaccactagtttcctccaaattacaaccaccagaaatttagttctttcaataatgacttcaaagtttaa

SEQ ID NO: 718 - DNA, 401 bp, Homo sapiens
gagcaatattgccgccaatgcaaatgcatgtcctgcagcataaagatacaccagaacagaacaaaccccagaagctgaaagtcccatgaagcccaattgagatggatggagacagagggagcatgagacaaacaaagtttaaaaatgaattaattgcaatcattcataaggtcaaatgccaactagacatcccaactccaytgggtgtgtgtgaatactacaaagaacatagactccatccctaactttggaacagagcgaatcaactccgtttggagacctaagcagagaccacataggctggttagcgaacaatgaagtcctccattagaggaggattgcaggtgcaatagtcagagatggagcaatcactgtgagctggtgggggtggaaaacaaggc

SEQ ID NO: 719 - DNA, 401 bp, Homo sapiens
tcaacgatgcccgcgttcgggactggggtggacaatgcgcaaaagcgttaggaaaaaagttcgcgcccgactgtgggttctgtgcctgcggtgttcttgacgctttggggacatttgggaggcaaagggtgcaccttcctgggcccgccgcgcgggcgcgaagaggacctaggcgagagttggctgtgcgggcctggggargcgagtctgtcctccgggtgcaattccccaacccgaaaccagagcccgggtcattagcgggaaaccgaagtggcgcggggatcgcatttcaaaagctcaaagagacaggctgactgggagcgctggaattggggcgggggggtgggggggcgatggtgggggtcggggatggtgaggggaagagccggaccgcccggaca

SEQ ID NO: 720 - DNA, 401 bp, Homo sapiens
accagtcatataggattaaggccccacctgaacagccttcatttaacttgaggcgctatttccaaaaacagtcgcatagactatggcccacccataatgatctcatttaaccttaataaccccttaaaaggccctatctccaaacatggttacattctgagctactgggggttgagacttcaacatatatatttgtggggrcacaattccacccgtgacaggcggatctacacttcaggcatgtctgaatccaggcatgcagatggcttcaccaagagtctgtctgtccccaccctatggctctgtttccttctggttaacgtgagctctggaggctctcttcaccttacaggcaagatggccagccccagattcacgtcctcctctcttagctaactcca

SEQ ID NO: 721 - DNA, 401 bp, Homo sapiens; identical to SEQ ID NO: 2

SEQ ID NO: 722 - DNA, 401 bp, Homo sapiens
tgcaaagggctccacagacccccgccctggagagaggagtctgagcctggcttaataacaaactgggatgtggctgggggcggacagcgacggcgggattcaaagacttaattccatgagtaaattcaacctttccacatccgaatggatttggattttatcttaatattttcttaaatttcatcaaataacattcaggastgcagaaatccaaaggcgtaaaacaggaactgagctatgtttgccaaggtccaaggacttaataaccatgttcagagggatttttcgccctaagtactttttattggttttcataaggtggcttagggtgcaagggaaagtacacgaggagaggactgggcggcagggctatgagcacggcaaggccaccggggagagag

SEQ ID NO: 723 - DNA, 401 bp, Homo sapiens
acaaaatggagaaataggaccttccacacagggtgcatgtgaggatgaaaggagtgaatgtataaagacacctttcccgataacccatactaccctcattatgactcttaacattagagagggctaaacaagggcagtgagaagactggcaggcagcagaaatcattttctttttgatctcacacaccagggcattccccstaagaggcagtgagatgtagcctggccctggaactagaaacagaggtggctgctgtgctagtgcctgagatgcacgatgctggggtcaaagacttgtgtccccacctcaggctgccaggatggcgtgtgtgccgtgacagggctcccgagtcatgcactcaggcagacagcaggctggagcccagcattcaggggtggct

SEQ ID NO: 724 - DNA, 401 bp, Homo sapiens
acggtgctcatgtgcctgctctctaagcgctgccctctatgcgctgcataaacaatcttccaggggcctttcaccagtgacagtccagaggctcagcaggaccccgcagccagccaacggcaaacccagcacaccaccagggcccccctgtcacaaccctcgcacactcgaaggcagtgcttcccagcacgggtaaaagcwccgtgtcaaatgtcagcaatggcccggtccattagggtgcaggggctgcggggccagtcttggggtcagtgtctcgccagctgcacaaccagcgggcagaggtgtcactcaccatcaggtccttcacgcggttgctgagctggtcgatgaacaggatgggcaggtaatgcacggttttccccagctggatcctgggatta

SEQ ID NO: 725 - DNA, 401 bp, Homo sapiens; identical to SEQ ID NO: 724

SEQ ID NO: 726 - DNA, 401 bp, Homo sapiens
ctttaagaaataatgtctaaaagtttttcaaatatgaggaaaaacataaaaccacagatccaagaagctcaacaaaacaaagcacaagaaacaggaagaaattaaaagttatatcacagtcaaattgctgaaaaccagcaacaaagagaatatcttaagagtatcagaggaaaagagattaatgacaggccaagaaacaaygaaaacaatacagatttcttgtaggaaacacaagacaaaagacattttttaaaaccaaaaggaaaaaaaatgctacattaaaatgttttttacccactgaaagtatatttcaaaacatattttaggccaggcttggtggctcacacctgtaatcccagcactttgggaggccaaggtgggtggatcgcttaaggtcagga

SEQ ID NO: 727 - DNA, 401 bp, Homo sapiens
tgaacccaagatctgcttcacctgctgtgtgacttttggcatgttacataatctttctgatccttagtttcctcagtgcaaaatgataatgacactctactgtgccggtgtgaagattaggtgagttagtaaccacaggttcctagaatatggtgggctctcacgtatttagaaagaagaggcacatcttgataacatgcygagaactgaggcaactgatgtctttaggctcttatagtcccagcatgtttttaagagctactgaatttgaagagaccatatcagtttcatctgctcaaattagatgaatttgcagttcatatcccaaggtttgttgttcttgttttttataggacgtgcaatccctggtataaaataagttgaaatccagttgactcatc